SEQUENCE LISTING (215 sequences)

SEQ ID NO: 1 - protein, 101 aa, C. sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 2 - DNA, 303 bp, Artificial Sequence
  Synthetic
atggcagtgaagcatttgattgtattgaagttcaaagatgaaatcacagaagcccaaaaggaagaatttttcaagacgtatgtgaatcttgtgaatatcatcccagccatgaaagatgtatactggggtaaagatgtgactcaaaagaataaggaagaagggtacactcacatagttgaggtaacatttgagagtgtggagactattcaggactacattattcatcctgcccatgttggatttggagatgtctatcgttctttctgggaaaaacttctcatttttgactacacaccacgaaag

SEQ ID NO: 3 - DNA, 78 bp, Artificial Sequence
  Synthetic
aattgctcagcattttccttttggtttgtttgcaaaataatatttttctttctctcattcaatatccaaatttcaata

SEQ ID NO: 4 - protein, 27 aa, C. sativa
NCSAFSFWFVCKIIFFFLSFNIQISIA

SEQ ID NO: 5 - protein, 719 aa, C. sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQFSHFE

SEQ ID NO: 6 - protein, 543 aa, C. sativa
MEKSGYGRDGIYRSLRPPLHLPNNNNLSMVSFLFRNSSSYPQKPALIDSETNQILSFSHFKSTVIKVSHGFLNLGIKKNDVVLIYAPNSIHFPVCFLGIIASGAIATTSNPLYTVSELSKQVKDSNPKLIITVPQLLEKVKGFNLPTILIGPDSEQESSSDKVMTFNDLVNLGGSSGSEFPIVDDFKQSDTAALLYSSGTTGMSKGVVLTHKNFIASSLMVTMEQDLVGEMDNVFLCFLPMFHVFGLAIITYAQLQRGNTVISMARFDLEKMLKDVEKYKVTHLWVVPPVILALSKNSMVKKFNLSSIKYIGSGAAPLGKDLMEECSKVVPYGIVAQGYGMTETCGIVSMEDIRGGKRNSGSAGMLASGVEAQIVSVDTLKPLPPNQLGEIWVKGPNMMQGYFNNPQATKLTIDKKGWVHTGDLGYFDEDGHLYVVDRIKELIKYKGFQVAPAELEGLLVSHPEILDAVVIPFPDAEAGEVPVAYVVRSPNSSLTENDVKKFIAGQVASFKRLRKVTFINSVPKSASGKILRRELIQKVRSNM

SEQ ID NO: 7 - protein, 385 aa, C. sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 8 - protein, 307 aa, Unknown [sequence omitted]

SEQ ID NO: 9 - DNA, 924 bp, Artificial Sequence
  Synthetic
atgtcagaagccgcagatgtcgaaagagtttacgccgctatggaagaagccgccggtttgttaggtgttgcctgtgccagagataagatctacccattgttgtctacttttcaagatacattagttgaaggtggttcagttgttgttttctctatggcttcaggtagacattctacagaattggatttctctatctcagttccaacatcacatggtgatccatacgctactgttgttgaaaaaggtttatttccagcaacaggtcatccagttgatgatttgttggctgatactcaaaagcatttgccagtttctatgtttgcaattgatggtgaagttactggtggtttcaagaaaacttacgctttctttccaactgataacatgccaggtgttgcagaattatctgctattccatcaatgccaccagctgttgcagaaaatgcagaattatttgctagatacggtttggataaggttcaaatgacatctatggattacaagaaaagacaagttaatttgtacttttctgaattatcagcacaaactttggaagctgaatcagttttggcattagttagagaattgggtttacatgttccaaacgaattgggtttgaagttttgtaaaagatctttctcagtttatccaactttaaactgggaaacaggcaagatcgatagattatgtttcgcagttatctctaacgatccaacattggttccatcttcagatgaaggtgatatcgaaaagtttcataactacgctactaaagcaccatatgcttacgttggtgaaaagagaacattagtttatggtttgactttatcaccaaaggaagaatactacaagttgggtgcttactaccacattaccgacgtacaaagaggtttattgaaagcattcgatagtttagaagactaa

SEQ ID NO: 10 - protein, 395 aa, C. sativa
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 11 - protein, 398 aa, C. sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 12 - protein, 323 aa, C. sativa
MSAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 13 - protein, 544 aa, C. sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 14 - protein, 545 aa, C. sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 15 - protein, 545 aa, C. sativa
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAACKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 16 - protein, 18 aa, Saccharomyces cerevisiae
KFISTFLTFILAAVSVTA

SEQ ID NO: 17 - protein, 4 aa, Saccharomyces cerevisiae
HDEL

SEQ ID NO: 18 - DNA, 54 bp, Artificial Sequence
  Synthetic
aagtttatcagtaccttcttgacctttatcttggccgctgtctccgtaaccgct

SEQ ID NO: 19 - DNA, 12 bp, Artificial Sequence
  Synthetic
catgatgaatta

SEQ ID NO: 20 - protein, 545 aa, C. sativa
MNCSAFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHH

SEQ ID NO: 21 - protein, 517 aa, C. sativa
NPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHH

SEQ ID NO: 22 - DNA, 1551 bp, Artificial Sequence
  Synthetic
aacccgcaagaaaactttctaaaatgcttttctgaatacattcctaacaaccctgccaacccgaagtttatctacacacaacacgatcaattgtatatgagcgtgttgaatagtacaatacagaacctgaggtttacatccgacacaacgccgaaaccgctagtgatcgtcacaccctccaacgtaagccacattcaggcaagcattttatgcagcaagaaagtcggactgcagataaggacgaggtccggaggacacgacgccgaagggatgagctatatctcccaggtaccttttgtggtggtagacttgagaaatatgcactctatcaagatagacgttcactcccaaaccgcttgggttgaggcgggagccacccttggtgaggtctactactggatcaacgaaaagaatgaaaattttagctttcctgggggatattgcccaactgtaggtgttggcggccacttctcaggaggcggttatggggccttgatgcgtaactacggacttgcggccgacaacattatagacgcacatctagtgaatgtagacggcaaagttttagacaggaagagcatgggtgaggatcttttttgggcaattagaggcggagggggagaaaattttggaattatcgctgcttggaaaattaagctagttgcggtaccgagcaaaagcactatattctctgtaaaaaagaacatggagatacatggtttggtgaagctttttaataagtggcaaaacatcgcgtacaagtacgacaaagatctggttctgatgacgcattttataacgaaaaatatcaccgacaaccacggaaaaaacaaaaccacagtacatggctacttctctagtatatttcatgggggagtcgattctctggttgatttaatgaacaaatcattcccagagttgggtataaagaagacagactgtaaggagttctcttggattgacacaactatattctattcaggcgtagtcaactttaacacggcgaatttcaaaaaagagatccttctggacagatccgcaggtaagaaaactgcgttctctatcaaattggactatgtgaagaagcctattcccgaaaccgcgatggtcaagatacttgagaaattatacgaggaagatgtgggagttggaatgtacgtactttatccctatggtgggataatggaagaaatcagcgagagcgccattccatttccccatcgtgccggcatcatgtacgagctgtggtatactgcgagttgggagaagcaagaagacaacgaaaagcacattaactgggtcagatcagtttacaatttcaccaccccatacgtgtcccagaatccgcgtctggcttacttgaactaccgtgatcttgacctgggtaaaacgaacccggagtcacccaacaattacactcaagctagaatctggggagagaaatactttgggaagaacttcaacaggttagtaaaggttaaaaccaaggcagatccaaacaacttttttagaaatgaacaatccattcccccgctacccccgcaccatcac

SEQ ID NO: 23 - protein, 540 aa, Artificial Sequence
  Synthetic
MKFISTFLTFILAAVSVTANPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHHHDEL

SEQ ID NO: 24 - DNA, 1620 bp, Artificial Sequence
  Synthetic
atgaagtttatcagtaccttcttgacctttatcttggccgctgtctccgtaaccgctaacccgcaagaaaactttctaaaatgcttttctgaatacattcctaacaaccctgccaacccgaagtttatctacacacaacacgatcaattgtatatgagcgtgttgaatagtacaatacagaacctgaggtttacatccgacacaacgccgaaaccgctagtgatcgtcacaccctccaacgtaagccacattcaggcaagcattttatgcagcaagaaagtcggactgcagataaggacgaggtccggaggacacgacgccgaagggatgagctatatctcccaggtaccttttgtggtggtagacttgagaaatatgcactctatcaagatagacgttcactcccaaaccgcttgggttgaggcgggagccacccttggtgaggtctactactggatcaacgaaaagaatgaaaattttagctttcctgggggatattgcccaactgtaggtgttggcggccacttctcaggaggcggttatggggccttgatgcgtaactacggacttgcggccgacaacattatagacgcacatctagtgaatgtagacggcaaagttttagacaggaagagcatgggtgaggatcttttttgggcaattagaggcggagggggagaaaattttggaattatcgctgcttggaaaattaagctagttgcggtaccgagcaaaagcactatattctctgtaaaaaagaacatggagatacatggtttggtgaagctttttaataagtggcaaaacatcgcgtacaagtacgacaaagatctggttctgatgacgcattttataacgaaaaatatcaccgacaaccacggaaaaaacaaaaccacagtacatggctacttctctagtatatttcatgggggagtcgattctctggttgatttaatgaacaaatcattcccagagttgggtataaagaagacagactgtaaggagttctcttggattgacacaactatattctattcaggcgtagtcaactttaacacggcgaatttcaaaaaagagatccttctggacagatccgcaggtaagaaaactgcgttctctatcaaattggactatgtgaagaagcctattcccgaaaccgcgatggtcaagatacttgagaaattatacgaggaagatgtgggagttggaatgtacgtactttatccctatggtgggataatggaagaaatcagcgagagcgccattccatttccccatcgtgccggcatcatgtacgagctgtggtatactgcgagttgggagaagcaagaagacaacgaaaagcacattaactgggtcagatcagtttacaatttcaccaccccatacgtgtcccagaatccgcgtctggcttacttgaactaccgtgatcttgacctgggtaaaacgaacccggagtcacccaacaattacactcaagctagaatctggggagagaaatactttgggaagaacttcaacaggttagtaaaggttaaaaccaaggcagatccaaacaacttttttagaaatgaacaatccattcccccgctacccccgcaccatcaccatgatgaatta

SEQ ID NO: 25 - protein, 472 aa, Aspergillus niger
GNTTSIAGRDCLISALGGNSALAVFPNELLWTADVHEYNLNLPVTPAAITYPETAAQIAGVVKCASDYDYKVQARSGGHSFGNYGLGGADGAVVVDMKHFTQFSMDDETYEAVIGPGTTLNDVDIELYNNGKRAMAHGVCPTIKTGGHFTIGGLGPTARQWGLALDHVEEVEVVLANSSIVRASNTQNQDVFFAVKGAAANFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIANHTAGTPAWFVTLSLEGGAINDVAEDATAYAHRDVLFWVQLFMVNPVGPISDTTYEFTDGLYDVLARAVPESVGHAYLGCPDPRMEDAQQKYWRTNLPRLQELKEELDPKNTFHHPQGVMPA

SEQ ID NO: 26 - DNA, 1419 bp, Artificial Sequence
  Synthetic
ggtaatacgacctctattgccggcagagattgtttgatctcagctttaggtggtaactccgctcttgcagtttttccaaacgagttgctatggacagctgacgtacacgaatataatctgaacttgcctgtcactcccgctgctataacctacccagaaaccgccgctcagattgccggtgtggttaagtgcgcttctgattacgactataaagtccaagcaaggtccggaggtcatagtttcggtaattacggcttgggtggagctgacggtgcagttgtcgttgatatgaagcacttcactcaattttcgatggacgatgaaacttacgaagctgttatcggtccaggtacaactttaaacgatgtcgacatcgaattgtacaacaacggtaaaagagccatggctcatggtgtatgtccaaccattaagactggtggtcacttcaccatcggtggtctaggacctacggctcgtcaatggggtctggctttggaccatgtcgaggaagttgaagttgtgttagctaactctagcattgttagagcctctaatacacaaaatcaagatgttttctttgcagtcaagggtgctgctgctaacttcggaatcgtcactgaatttaaagttagaactgaaccagccccaggtttggctgtacagtactcctataccttcaacttgggttcaactgccgagaaggctcaattcgttaaggattggcaatctttcatttcggctaagaacctaaccagacaattttataataacatggtcatttttgatggtgacataatcttggaaggtttattcttcggtagcaaggaacaatacgacgccttgggccttgaagatcacttcgcaccaaagaatccaggtaacatattggttttaacagattggctaggcatggtgggtcacgcattggaagacactattttaaaattggtcggtaataccccaacatggttctatgctaagtccttgggttttagacaagacactctgatcccttctgccggtattgacgaatttttcgaatacattgctaaccataccgccggcactcctgcttggtttgttactttgtccttagagggtggtgctatcaacgatgtcgcagaagatgctacggcctatgctcacagagatgttttgttctgggtccaactattcatggttaatccagtcggtcctatctctgacactacctacgagtttacagacggcttgtacgatgtgttggcccgtgctgttccagaaagcgtgggacatgcttaccttggttgtccagatccaagaatggaagacgctcaacagaagtattggcgtaccaatttgccccgtctgcaagaactaaaggaagagttggatccaaaaaacaccttccatcacccacagggtgttatgccagcttaa

SEQ ID NO: 27 - protein, 473 aa, Aspergillus niger
MGNTTSIAGRDCLISALGGNSALAVFPNELLWTADVHEYNLNLPVTPAAITYPETAAQIAGVVKCASDYDYKVQARSGGHSFGNYGLGGADGAVVVDMKHFTQFSMDDETYEAVIGPGTTLNDVDIELYNNGKRAMAHGVCPTIKTGGHFTIGGLGPTARQWGLALDHVEEVEVVLANSSIVRASNTQNQDVFFAVKGAAANFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIANHTAGTPAWFVTLSLEGGAINDVAEDATAYAHRDVLFWVQLFMVNPVGPISDTTYEFTDGLYDVLARAVPESVGHAYLGCPDPRMEDAQQKYWRTNLPRLQELKEELDPKNTFHHPQGVMPA

SEQ ID NO: 28 - DNA, 1422 bp, Artificial Sequence
  Synthetic
atgggtaatacgacctctattgccggcagagattgtttgatctcagctttaggtggtaactccgctcttgcagtttttccaaacgagttgctatggacagctgacgtacacgaatataatctgaacttgcctgtcactcccgctgctataacctacccagaaaccgccgctcagattgccggtgtggttaagtgcgcttctgattacgactataaagtccaagcaaggtccggaggtcatagtttcggtaattacggcttgggtggagctgacggtgcagttgtcgttgatatgaagcacttcactcaattttcgatggacgatgaaacttacgaagctgttatcggtccaggtacaactttaaacgatgtcgacatcgaattgtacaacaacggtaaaagagccatggctcatggtgtatgtccaaccattaagactggtggtcacttcaccatcggtggtctaggacctacggctcgtcaatggggtctggctttggaccatgtcgaggaagttgaagttgtgttagctaactctagcattgttagagcctctaatacacaaaatcaagatgttttctttgcagtcaagggtgctgctgctaacttcggaatcgtcactgaatttaaagttagaactgaaccagccccaggtttggctgtacagtactcctataccttcaacttgggttcaactgccgagaaggctcaattcgttaaggattggcaatctttcatttcggctaagaacctaaccagacaattttataataacatggtcatttttgatggtgacataatcttggaaggtttattcttcggtagcaaggaacaatacgacgccttgggccttgaagatcacttcgcaccaaagaatccaggtaacatattggttttaacagattggctaggcatggtgggtcacgcattggaagacactattttaaaattggtcggtaataccccaacatggttctatgctaagtccttgggttttagacaagacactctgatcccttctgccggtattgacgaatttttcgaatacattgctaaccataccgccggcactcctgcttggtttgttactttgtccttagagggtggtgctatcaacgatgtcgcagaagatgctacggcctatgctcacagagatgttttgttctgggtccaactattcatggttaatccagtcggtcctatctctgacactacctacgagtttacagacggcttgtacgatgtgttggcccgtgctgttccagaaagcgtgggacatgcttaccttggttgtccagatccaagaatggaagacgctcaacagaagtattggcgtaccaatttgccccgtctgcaagaactaaaggaagagttggatccaaaaaacaccttccatcacccacagggtgttatgccagcttaa

SEQ ID NO: 29 - protein, 495 aa, Artificial Sequence
  Synthetic
MKFISTFLTFILAAVSVTAGNTTSIAGRDCLISALGGNSALAVFPNELLWTADVHEYNLNLPVTPAAITYPETAAQIAGVVKCASDYDYKVQARSGGHSFGNYGLGGADGAVVVDMKHFTQFSMDDETYEAVIGPGTTLNDVDIELYNNGKRAMAHGVCPTIKTGGHFTIGGLGPTARQWGLALDHVEEVEVVLANSSIVRASNTQNQDVFFAVKGAAANFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIANHTAGTPAWFVTLSLEGGAINDVAEDATAYAHRDVLFWVQLFMVNPVGPISDTTYEFTDGLYDVLARAVPESVGHAYLGCPDPRMEDAQQKYWRTNLPRLQELKEELDPKNTFHHPQGVMPAHDEL

SEQ ID NO: 30 - DNA, 1488 bp, Artificial Sequence
  Synthetic
atgaagtttatcagtaccttcttgacctttatcttggccgctgtctccgtaaccgctggtaatacgacctctattgccggcagagattgtttgatctcagctttaggtggtaactccgctcttgcagtttttccaaacgagttgctatggacagctgacgtacacgaatataatctgaacttgcctgtcactcccgctgctataacctacccagaaaccgccgctcagattgccggtgtggttaagtgcgcttctgattacgactataaagtccaagcaaggtccggaggtcatagtttcggtaattacggcttgggtggagctgacggtgcagttgtcgttgatatgaagcacttcactcaattttcgatggacgatgaaacttacgaagctgttatcggtccaggtacaactttaaacgatgtcgacatcgaattgtacaacaacggtaaaagagccatggctcatggtgtatgtccaaccattaagactggtggtcacttcaccatcggtggtctaggacctacggctcgtcaatggggtctggctttggaccatgtcgaggaagttgaagttgtgttagctaactctagcattgttagagcctctaatacacaaaatcaagatgttttctttgcagtcaagggtgctgctgctaacttcggaatcgtcactgaatttaaagttagaactgaaccagccccaggtttggctgtacagtactcctataccttcaacttgggttcaactgccgagaaggctcaattcgttaaggattggcaatctttcatttcggctaagaacctaaccagacaattttataataacatggtcatttttgatggtgacataatcttggaaggtttattcttcggtagcaaggaacaatacgacgccttgggccttgaagatcacttcgcaccaaagaatccaggtaacatattggttttaacagattggctaggcatggtgggtcacgcattggaagacactattttaaaattggtcggtaataccccaacatggttctatgctaagtccttgggttttagacaagacactctgatcccttctgccggtattgacgaatttttcgaatacattgctaaccataccgccggcactcctgcttggtttgttactttgtccttagagggtggtgctatcaacgatgtcgcagaagatgctacggcctatgctcacagagatgttttgttctgggtccaactattcatggttaatccagtcggtcctatctctgacactacctacgagtttacagacggcttgtacgatgtgttggcccgtgctgttccagaaagcgtgggacatgcttaccttggttgtccagatccaagaatggaagacgctcaacagaagtattggcgtaccaatttgccccgtctgcaagaactaaaggaagagttggatccaaaaaacaccttccatcacccacagggtgttatgccagcttaacatgatgaatta

SEQ ID NO: 31 - protein, 27 aa, C. sativa
NCSTFSFWFVCKIIFFFLSFNIQISIA

SEQ ID NO: 32 - DNA, 81 bp, Artificial Sequence
  Synthetic
aattgctcaacattctccttttggtttgtttgcaaaataatatttttctttctctcattcaatatccaaatttcaatagct

SEQ ID NO: 33 - protein, 517 aa, C. sativa
NPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAACKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 34 - DNA, 1419 bp, Artificial Sequence
  Synthetic
atgggcaatggacaatccaccccactgcaacagtgtttaaacacggtatgcaacggtcgtcttggttgtgtcgctttcccttcggatgcattgtaccaagccgcttgggtgaagccatataatttggacgttcccgttactccaatcgctgtctttaaaccatcttctactgaagacgttgccggtgctattaagtgtgctgtcgcaagcaacgttcatgttcaagctaagtcaggtggtcacagttacgctaacttcggtttgggtggtcaagatggtgagttaatgatagacttggccaatctacaagattttcacatggataaaacctcctggcaggctaccttcggcgctggttacaggttgggtgacctagataagaagttgcaagcaaacggaaacagagccattgctcatggtacatgtccaggtgtaggtatcggaggtcacgctactattggtggtttaggtcctatgtcaagaatgtggggctctgctctggatcatgtcttgtccgttcaagtcgttactgccgacggttctatcaaaaatgcatcagaatctgaaaattctgacttgttctgggctttgagaggtgctggtgccagttttggtgtcatcacaaagttcactgttaagacccacccagccccaggttccgtggttcaatatacttacaaaatttcgttaggatctcaggctcaaatggctcctgtttatgctgcctggcaagcattagctggtgacgctaagttggatagaagattctcaaccctttttattgctgaaccattgggagccttaataacaggtactttttacggtacaaaggccgaatatgaagctaccggtattgctgcaagacttccatccggcggtaccttggacctaaagttattggattggttgggtagcttggctcatatcgctgaagttgtcggtctgactttaggtgatattcctacttctttctacggtaaatcgttggccttgagggaagaagacatgttggatagaacatccatcgacggtttgtttcgttacatgggagatgcagatgctggtacgctattgtggttcgtgatattcaactctgagggtggcgctatggccgatactccagctggtgccactgcttaccctcacagagataagttgattatgtatcaatcttatgtgatcggtattccaacgcttactaaagcaactagagactttgctgacggtgtacacgatagagtccgtatgggagctccagccgctaacagtacctacgctggttatatcgacagaaccttatcaagagaagccgctcaagagttttactggggcgctcagttaccaagactaagggaagttaagaaggcttgggaccctaaagacgttttccataatccacaatccgtcgatccagctgaa

SEQ ID NO: 35 - DNA, 1419 bp, Artificial Sequence
  Synthetic
atgggaaatacaacttcaattgcaggcagagattgcttgatcagtgctctaggtggtaactctgccttagctgtgtttcctaaccaacttctgtggacggccgacgtccatgagtataatttgaacttgccagttactccagctgctataacctacccagaaaccgctgaacagattgccggtatcgttaaatgtgcttccgattacgactataaggtccaagctcgttctggtggtcactcgttcggtaactacggtttaggaggtactgatggcgcagttgtagttgacatgaagcacttcaaccaatttagcatggacgatcaaacctacgaagctgtcattggtcccggtactaccttgaatgatgtagacatcgaattgtataacaatggtaaaagagctatggcacatggtgtttgtccaactataaagacaggtggacacttcacaattggtggtttaggacctactgccagacaatggggtctagctttggaccacgttgaggaagtcgaagttgtcttggctaattcctctatcgttagggcttcaaacacccagaaccaagatgtgttctttgctgtaaagggtgccgctgctgacttcggtattgtcacggaatttaaagtcagaactgaaccagccccaggtcttgccgtccaatactcttacaccttcaacctaggttcgactgctgaaaaggctcaattcgttaaggattggcaatctttcatttccgccaagaatttgacgagacaattttataacaacatggttatctttgacggtgatattatcttggaaggtttattctttggcagtaaagaacaatacgatgcattaggtttggaagaccatttcgctcccaagaatccaggtaatatcttggttttaaccgattggctaggtatggtgggacatgccttagaggacactatattgaagttggttggcaacactccaacatggttttacgctaaatccttgggtttcaggcaggatactttaattccaagtgctggtatcgatcaatttttcgaatacattgctaaccacaccgctggtactcctgcatggttcgtaaccttgtctctggagggtggtgccatcaatgacgttgctgaagacgccactgcttatgctcacagagatgtcctattctgggtccaacttttcatggttaacccattgggtccaatttctgaaacaacttacgaatttaccgatggattgtacgacgtgctagcacgtgcagttccagaaagcgtcggtcacgcttatttgggttgtcctgatccaagaatggagaacgcccctcaaaagtattggagaacgaatcttccaagacttcaagaactgaaggaagagttggatccaaagaacacttttcatcatcctcaaggtgtcatcccagct

SEQ ID NO: 36 - DNA, 1419 bp, Artificial Sequence
  Synthetic
atgggaaacacgaccagcatagctggtcgtgactgtctgatctctgccttgggtggcaattcagcattagctgctttcccaaaccaactattgtggactgccgatgtccacgaatacaaccttaatttgcctgtgacaccagctgctattacttatcccgagactgccgaacagatcgctggtattgttaagtgcgcctctgattacgactacaaagtacaagctagatcgggtggtcattcctttggtaattatggtttgggtggtaccgatggtgctgtcgttgttgacatgaagcacttcaaccaattttctatggatgatcaaacctacgaagcagtcattggaccaggtactaccttaaacgacgtagatatcgaattgtacaataacggtaaaagagctatggcccatggtgtgtgtccaacaatcaagactggaggtcacttcaccattggcggcttgggtccaactgctagacaatggggtttagctttagaccatgttgaagaggttgaagttgtcttggccaactccagtattgttagggcatctaatactcaaaaccaggacgttttctttgctgtcaagggtgctgctgctgacttcggtatcgtgaccgaatttaaagttagaacagaacctgccccaggtttggccgtccaatattcctacaccttcaatcttggttcaactgctgaaaaggcacaattcgtaaaggattggcaatacttcatctctgctaaaaacctaacaagacaattttacaacaacatggttatttttgacggtgatataattttggaaggtctgttcttcggtagtaaggaacaatatgacgccttgggtttggaggatcactttgctcccaagaatccaggaaatattttagtcctaacggattggttgggcatggttggtcacgcattagaagatactattctaaaattggtcggtaacacgccaacttggttctatgctaagtccttgggttttcgtcaggacacccttatcccttctgctggtattgatgaatttttcgagtacatcgctaatcataccgccggtactccagcttggtttgttactttatctttggaaggtggagctatcaacgacgtcgctgaagatgccacagcatacgcacatagagatgtgttattctgggttcaattgttcatggttaaccctcttggtccaatttcagaaacaacttatgaatttaccgatggattgtacgacgttttagctagagctgtcccagaatctgtaggtcacgcttacttgggttgtccagacccaagaatggagaacgcacctcaaaagtattggaggacaaacttgccaagactacaggaactgaaagaggaattggaccccaagaatacttttcaccatccacaaggtgttatcccagct

SEQ ID NO: 37 - DNA, 1539 bp, Artificial Sequence
  Synthetic
atggatccaatcgaggacgccattttgcagtgcttaagcctacacagtgacccttcgcatccaatatcaggcgtaacgtatttccccaatacaccatcttacattcctatcctgcactcctacattcgtaaccttagatttacctctccatccactagaaaaccattgttcatcgttgctccaactcatatatctcacatccaagcatcaattatctgttgtaagtcttttcaattgcaaattaggattagaagtggaggtcacgattatgatggtttgtcctacgtcagccaatctcccttcgctattatggacatgttcgctatgagatccgttgaagtcaacttagaagatgaaaccgtttgggttgactctggttccactatcggtgaattgtaccatggtattgccgaaagatctaaggtccatggtttcccagctggtgtgtgtcactcagttggcgtcggtggacacttttccggtggtggttatggtaatatgatgagaaagttcggtttgtctgtggaccatgttctggatgctgttatcgttgatgcagaaggccgtgtcttagacagaaaaaagatgggtgaagacctattctggggtataagaggtggtggtggcgcttcgtttggtgttatcgtcagttggagaattaaattggtcccagtgcctgaggttgtaaccgtcttccgtgttttgaagaccttggaacaaggtgccacagatgtcgttcacagatggcaatacgtcgccgacaacatccacgatgacttatttattagagttgttctatctccagttaagagaaaaggtcagaagactatcagagctaagtttaatgctttgttcttgggtaacgctcaagaattactgcgtgtcatgtctgattcttttccagaattgggattagtgggtgaagactgtatcgagatgagctggattgactccgtattgttctgggataactttccagtaggtacatctgtagatgttttattgcagcgtcacgacactcctgaaaaattcttgaagaagaaatccgattacgttcaacaaccaatctctaagactggattagaaggtgtttggaataaaatgatggaacttgaaaagccagtgttgaccttgaatccatatggtggtagaatgggtgaaataagtgaaatggaaattccttttccacatagagctggtaacttgtacaagatccaatactcggtcaactggaaggaggaaggtgaggatgttgcaaacaggtatcttgacctgattagaatgttatacgactacatgaccccatatgtttcaaagtcccccagatcaagttatttgaactacagagatgtcgatataggagtcaatggtccaggcaatgccacttatgctgaagctagagtctggggagagaaatacttcaagagaaactttgacagattggttgaagtcaaaactagggttgatccaagtaacttcttcaggtacgaacaatctataccttccttggccgcttcgagcctaggtattatgtcggaa

SEQ ID NO: 38 - DNA, 1518 bp, Artificial Sequence
  Synthetic
atggtccacaatatattactacttggtttgatgccactgttggttcgtgcatcacctttgccaatttatcataactaccccccacaatcgactatcaacgactgcttgcaggccgctgatgttccagctatcttacaaagctctgcttcctttgatgccttgagtcaacctctaaattccagattaaaatctaagccagctgtgattacaatccctacgaccgctttgcacgtcagttctgctgttaagtgtgccgcacaattcaagctgaaagtaactccaagaggcggtggacattcttacaacgcacaatccttaggtgacggtgctgtcgttattgatatgcaacagttccacgacgttgtctacgactctaagactcaactagctaggattggtggtggagctagattgggtaacgttgcccaaaaattgtatgatcaaggtaagagagctatgccacatggtacctgtccagatgtcggtattggcggtcactccgccggtggttttggttggacctcacgtcagtggggtatcactgtagatcacatagacgaggttgaagtggtaacagctgacggttctatcagaagagctaataaggatcaaaattccgatttgttctgggcattgagaggagctgccccatcgttcggtgttattactaacttttggttttctaccttggaagctcctgattctaacgttatttacagttataagttcactggtttatctttagacgaaatcagtacagctttgttggaagtgcaaaagttcggtcaaaccgctcccaaagaagtcggcatgcttatccaaatattagacaatggttctggtttcagattgtacggtacgtactataacactacccgtcaacaatttgataatttattcggccaacttttgcaaagattgccatccccaggtaacagcgctgaggtttctgtcaagggttggattgactcgttgatatttgcctctggcggtagcaagggtcttactgttccagaactgggtggaactaaccagcattcttccttttacacaaaatcattgatgactgctcaagattacccattaaccctggattcaattaagtccgtgttcaagtatgccatgaaccaaggtagagccgccaccgaaaggggtctaccatggatggtatttatctctttgttgggtggtagatatagcactctaccaacgccttccgctgcttcagataactctttctacggcagaaacactttgtgggctttttctttcaccgcttacctaggtaacgtcacagaacaaagcaatagagactcaatttacttcttgaatggtttcgacacttccgtaagaagatccgttgacaccgcttacatcaacggtcacgatactgaatattcgagagaagaagcacatagattatactacggtgacaaatatcaaaggttgtctgtcttaaagaagcaatgggatcctgagcaagttttctggtatccacaatccatcgaccccgccaat

SEQ ID NO: 39 - DNA, 1428 bp, Artificial Sequence
  Synthetic
atgggtaacacaacttcaatcgcagctggcagggattgcttactgtccgccgtcggaggtaatcacgctcatgttgcttttcaggaccaattgctatatcaagctaccgcagtggaaccatacaacttgaatattcccgttacgccagccgctgttacctaccctcaatcggctgatgaggttgccgctgtcgtaaaatgtgcagccgactatggttacaaggtgcaagctagaagcggtggtcacagtttcggtaactacggtttaggcggtgaagacggtgctatagtcgttgatatgaagcatttcgatcaattttctatggacgaatctacttatactgctactattggtccaggtatcaccttgggagacttggataccgccctatacaatgctggccatagagccatggctcacggtatttgtccaacaattcgtactggtggtcaccttaccatcggaggtttgggtccaactgctagacaatggggtttggccttagatcacgttgaagaagtcgaagttgtcttggcaaacagctccatcgtcagagcatcagacactcagaaccaagagatcttgttcgctgttaagggtgctgctgcttctttcggtatagtaactgaatttaaagttagaacagaagaagctcctggtcttgccgtccaatactccttcaccttcaacttaggtacagctgccgagaaggctaaattggttaaggactggcaagcttttattgcacaagaagatttgacgtggaagttctactctaacatgaatattatcgacggtcaaattatcctagaaggcatatatttcggttctaaggctgaatacgatgccttaggtttggaggaaaagtttccaaccagtgaaccaggcactgttttagtcttgacggactggctgggtatggttggtcatggtttggaagatgttattttgcgtttagtaggcaatgctccaacttggttctatgctaaatctttaggttttgctcccagggcattgatcccagattccgctattgacgatttcttcgaatacattcacaagaacaatcctggtaccgttagttggttcgtcacactatcgttggaaggtggtgcaataaacaaggtgccagaagatgccactgcttacggacatagagatgttttgttttgggttcaaatctttatgattaacccactaggtcctgtttctcagaccatttacgactttgccgacggtctttatgacgttctggctaaagccgtccccgaatccgcaggtcatgcttatttgggctgtccagacccaagaatgccaaatgctcaacaagcctactggagaaataacttgccaagactagaggaattgaagggtgacttagatccaaaggatatcttccacaacccacagggtgtcatggttgtctct

SEQ ID NO: 40 - DNA, 1422 bp, Artificial Sequence
  Synthetic
atgggtaacactaccagcattgccggccgtgactgcctagtttccgctttgggtggtaatgcaggtctggtggcttttcagtcacaaccattataccaaacaaccgctgtccatgagtataaccttaacatacccgttactccagccgctatcgcttaccctgaaactgccgaacaaattgctgctgtcgtaaaatgtgcatcggaatatgattacaaggttcaagcaagatccggtggtcactctttcggaaattacggtttgggtggtacggatggtgctgttgtggtcgacatgaagcacttcaaccaatttagtatggacgatcaaacctatgaagctgttatcggcccaggtactactttgggcgacgtcgatactgagctatacaataacggtaagagagccatggcccatggtatctgtccaacaatttctaccggtggccacttcacgatgggtggtttaggtccaacggctagacagtggggtttggcattggatcacgttgaagaagtagaagtcgttttggctaattcttctatcgtgagggcttccaacacccaaaaccaagaagttttctttgccgttaaaggagctgctgcttcatttggtattgtcaccgaatttaaggttagaactcaaccagctcctggattggctgtccaatactcttacactttcaacttgggttcgagtgctgaaaaggctcaattcgtcaaggattggcaatctttcatctctgctaaaaacttaacaagacagttttataccaatatggttatattcgacggcgacattattttggaaggtctgttctttggtagcaaggagcaatacgaagcccttggtttggaagaacgtttcgtcccaaagaatcctggtaacattcttgttttaactgattggttgggtatggttggtcatgctttggaggacactatcttaagattagtcggtaacaccccaacctggttctacgcaaaatccctaggcttcaccccagatactttgataccctcctcaggtattgatgaatttttcgaatatatcgagaataataaggccggtacctctacatggtttgtaacattatctcttgaaggtggtgccatcaacgacgttccagctgatacgacagcatacggtcacagagatgtattgttttgggtccagatattcatggtttccccaactggtccagtttcctctacaacttacgattttgctgacggcttgtataacgtgttgactaaggcagttcctgaaagcgaaggtcatgcttacttgggatgtcctgaccctaagatggctaacgcccaacaaaaatattggagacaaaatctaccaagactggaggaattgaaagctactcttgacccaaaggatacctttcataacccccaaggtatcttgccagta

SEQ ID NO: 41 - DNA, 1449 bp, Artificial Sequence
  Synthetic
atgaatccttctataccctcaagctccatgggtaacacaacgtctatcgctggacgtgactgtttagttagtgccctgggtggtaacgctggtttggtagcattccaaaatcagccactataccaaaccactgctgtgcacgagtataacttaaacattccagtcactccagccgctattacctacccagaaactgctgaacaaatcgccgctgttgtcaaatgcgcatcccaatatgattacaaggttcaagctaggtctggtggccattcgtttggtaactacggtcttggtggcaccgatggtgctgttgtcgttgacatgaagtatttcaatcaattttccatggacgatcagacatacgaagcagttattggtcctggtactaccttgggagatgtcgatgtcgaattgtataacaatggtaaaagagctatggcccacggtgtgtgtccaactatctctaccggtggccatttcactatgggtggtttaggtccaacagctagacaatggggattggccttggaccacgttgaggaagttgaagtggttctagctaattcatctatcgtcagagcttcaaacacccaaaaccaagaagttttctttgccgtaaagggtgctgctgcctcgtttggtattgtcaccgaatttaaggttagaactcagcctgcaccaggtattgctgtgcaatactcttacactttcaacttgggttcctccgcagaaaaagctcaattcatcaaggactggcaatctttcgtttctgctaagaatcttacgagacaattctacactaacatggtcatatttgacggtgatattattttggaaggattgttcttcggtagtaaagagcaatatgaagccttgggtttagaagaaaggtttgtccctaagaacccaggtaatatcatggttctaacagattggttgggtatggttggccatgctctggaagatacgattttgagattggtaggtaatacgccaacttggttctacgctaagtccctgggttttactccagacacattaatccccagcagcggtattgacgaatttttcgaatatatagaaaacaacaaggcaggaaccagtacttggttcgtcaccctatctttggagggtggtgctattaacgatgtgccagcagatgctaccgcttacggtcacagagatgttttattctgggtacagatttttatggtttctcccactggtccagtttcctccactacctacgactttgccgacggcttgtacaatgtcttgactaaggctgttccagagtctgaaggccacgcctatttgggctgtccagaccctaaaatggctaacgctcaacaaaagtactggagacaaaacttgccacgtctggaagagcttaaggaaacattagatccaaaagacactttccacaatcctcaaggtatcttgccagca

SEQ ID NO: 42 - DNA, 1518 bp, Artificial Sequence
  Synthetic
atggcccgtgtccccgcactgttcttgggcctatcttcagtgtgtttgttagttttcttttgcagtttgattaacccaaccactcctttacatcagcacggtaagcaaacatctatcgctggtagggattgtcttgtatccgctttgggtggaaatactggtttagttgcttttcaagaccaattgttgtaccaaaccacggctgtccacgagtataacctgaacataccagttactccagccgctatcacctacccagaaacctcggaacaaattgctgccgttgttaaatgtgcatctgaatacgattataaggtccaggccagatccggtggtcatagcttcggtaattacggattgggtggtgctgacggtgctgttgtcgttgatatgaagcacttcactcaattttctatggacgaacaaacctatgaagctgtaattggcccaggtacaactctaggtgatgtggataccgagttgtacaacaacggtaaaagagctatggcccatggtatctgtcctactatctccactggcggtcacttcaccatgggtggtcttggcccaacagcaagacaatggggattggctcttgaccacgtcgaagaagtggaagttgttttggctaattcctctattgtcagagcatcaaacactcaaaatcaagaagttttctttgctgttaagggtgctgccgcctccttcggtatcgtcactgaatttaaagtcagaactcaaccagccccaggattagctgttcaatactcttatacgtttaacttgggtagctcagctgagaaggctcaattcgtcaaggactggcaatctttcatttctgctaagaatttaaccagacagttttacacaaacatggtaattttcgatggtaacataatcctagaaggcctatttttcggttccaaagaacaatacgaagctttgggtttggaggaccgttttgttccaaagaatcctggtaatatcttagtcttgacagattggctaggtatggttggccatgcattggaggacactattttgagattggtcggtaacacccccacatggttctacgcaaagagcttgggtttcactccagacacgttaatacctagttcaggtattgatgaatttttcgattatatagaaaacaacaaggctggtaccctgacgtggttcgttactttgagtttagaaggtggtgccattaacgatgtgccaaaggatgctactgcttatggtcacagagatgtgcttttttgggttcagatcttcatggcttctccaacaggtcctgtctcttccactacctacgacttcgccgatggtttgtataatgtcttgaccaaagccgttccagaatccgaaggacatgcttacctgggttgtccagacccaaagatggctgacgctcaacaaaagtactggaggcaaaacttacctagacttgaagaattgaaagcaaccttagatccaaaagatacttttcacaatccacaaggtatcttgccagca

SEQ ID NO: 43 - DNA, 1281 bp, Artificial Sequence
  Synthetic
atgacaaccaagtgtaggcatgcagtggaggctactgctctaggcacgatggtagcctacctgattagaaataacccacgtagacctagttcaactaacgctaatgttttggacacaggtttgggtggtgccgatggtgctgttgtcgtcgatatgaaacactttactcagttctccatggacgatgaaacctatgaagctgttatcggaccaggtaccactttaaacgacgttgatatagaattgtacaacaacggtaagagagctatggcacacggtgtctgccccaccattaagactggtggtcatttcaccatcggcggacttggtccaactgccagacaatggggtttggccttagatcacgttgaagaggttgaagtcgtgttggctaattcgtctattgtaagagctagcaacacacaaaaccaagacgttttctttgcagttaaaggtgctgccgctaatttcggaatcgtcactgaatttaaggtcagaaccgaaccagctcctggtttggctgttcaatattcctacactttcaatttaggttctactgctgaaaaggctcaattcgtgaaagattggcaatcttttatctctgctaagaatctaacgagacagttctacaacaacatggttatttttgacggtgatatcattcttgagggtttgtttttcggttccaaggaacaatatgacgccttgggcttggaagatcatttcgctccaaagaatccaggtaacatcttagtcctgacagactggctaggtatggttggacacgccttggaagacaccattttgaagcttgttggtaacaccccaacctggttctacgccaaatcgttgggttttagacaagatacattaattccatcagctggtatagacgaatttttcgaatacatagctaaccacactgctggtactcccgcatggttcgtcacacttagtttggaaggtggtgctattaatgatgtcgcagaagatgccacggcatatgctcaccgtgacgtcttgttctgggtacaactgtttatggtgaaccctttgggtcccatctctgacacgacttacgaatttactgacggtttatatgatgttttggcaagagctgttccagagtccgtgggccatgcttacttaggttgtccagatcctagaatggaagatgctcagcaaaagtattggcgtaccaacttgccaaggttgcaagagttgaaagaagaactagacccaaagaacactttccaccatccacaaggtgttatgccagcc

SEQ ID NO: 44 - DNA, 1425 bp, Artificial Sequence
  Synthetic
atgggtcatggacaatccactcccggtgagaactgtttaaatgcaatatgcggtaacagaacggactgtgtatcatacccattggatcctctattccacatcgcttgggcccgtccatataaccttcaggtgcctgttacaccagctgctgtcctgaggccagacaatgctcaagatgttgctgatgccgttaaatgtgccaacgaaaatggcattaaggtccaagctagatctggtggtcactcgtacggtaactttggtttgggaggtgacgacggtgcattggttttggatctagtcaatttacaaggtttcactatggacgaagaaaactggcatgctgctgttggcgcaggtattagattgggtaagctggatgaacacttgcataaaaatggtggtagagctatggctcacggtacctgtccaggcgtaggtatcggtggtcacgccaccatcggaggtttaggtccaatgagcagaatgtggggttctgccttggatcatgtcgtcgaagttgaagttgttactgctgacggctctattcaacgtgctaacgaaacagaaaatagtgatttattttgggccttgagaggtgctggtgcttccttcggtataatcactgagtttgtagttagaacccacccagaaccaggagatgtaattgaatatacctactctctgaccttcggttcacaagcagaaatggctcctatttaccaagaatggcaggaatttattggtgacccagacttggatagaaggttctcctctcaattcattgctcaaccattgggtgctatcatcactggaaccttctacggtactgaagacgaatatagagagagtggtatccccgataagataccaggaggtggtagtgatggtgttggtattttagttacagactggttgggttctctaggccaccaagccgaaagatccggtcttgctatttcggatcttccaagccctttttattctaagtcgttggccttcagaaaggaagacttgttgccaaaagagggtataactgagttattccagtacttgaacgacgctgaccatggtacattgatctgggtcgtcatctttaactccgaaggaggtgcaatgggtgatactgccgcaaacgctacagcctacccacatcgtgataagaccatgatgtatcaatcctacgtcattggtatccccgaagtgagtggcaccgcaagaagattcctggagggtgttcacgctaagattcaagatgctgctcctggtgctaattcaacgtatgctggttacatcgatacggaattaggcagagctgaagctcaagaagtgtactggggtagccagttgcctcaattgagaaagatcaaaaaggactgggacccaaaggacaggttttcaaacccacaatctgtccaagccgccaga

SEQ ID NO: 45 - DNA, 1548 bp, Artificial Sequence
  Synthetic
atgcgtgtcgttggaaagatgggtgctttgcaaagcactctggagaaatctatcaaggccgcattagctggtgacgatgatctatacgctgtgcccggtaaaccattttatcagatacaacatgtcaagccttacaacttgtcgattccaatcgaaccagccgctattacctatcctaagacaactgctcaagtagccgcaattatcaagtgcgctgttgctgctaatttgaaggtccaagccagatcaggtggccactcctacgctaactactgtattggtggtgtttctggtgctgttgttatcgaccttaaacacttccaaagattcagtatggatagaaccacgtggcaagcagccgtcggtgctggcactttattgggtaatttgaccaagaggatgcatgaagctggtaacagagccatggctcacggtacttgtccacaagtgggaattggtggtcacgcaaccataggtggccttggtccatcttcaagattgtggggtacggctttagaccatgttgaagaagtcgaaatagtcttggctgattccacaattaagagatgttctgctactcagaatccagacatcttttgggccgttaagggagctggtgcatccttcggtgttgtgactgaatttaaattaagaaccgagcccgaaccatctgaagctgtacatttctcttattcgttcactgttggttcctacgcaagcttggctgctgtttttaaatcatggcaatctttcgtcgctgacccaggtcttactcgtaagttctcctctgaagtcatcattacagagatcggtatgattatatcaggcacttattttggtagtcaagctgaatacgatgccctagatatgaagtctcaattgagaggtgacagtgttgctaagatcattgtttttaaggactggttaggattgttgggtcactgggccgaagatgtgggcctaagaattgccggtggtttacctgcccctttgtacgctaaaaccttgaccttcaacggtgccaacctgatcccagatgaagtcatcgataaattgttcgcctacctggacaaggttgaaaagggagctttggtatggttcgtcatttttgacctggctggaggtgccgttaatgacatagctcaagatgctacatcctatgctcatcgtgatgccttgttctacttgcagtcatatgcagtgggtttaggtaacgtttcacaaacaactaaggattttcttaccggtataaacacgactattaccaacggtatgccagaaggtggtgacttcggtgcttacccaggctacgttgacttggaattaccaaatggtccacacgcttactggagaaccaaccttccaaggttggaacaaatcaaagccctggtagatcctaatgatgtcttccacaacccacaatcttatttgtgcatcctatttttgctaaacttgctaaacagagctttggcttgggctccagttggtactgtccagccattccaagtcttaaggtactccattgacacaggtcctcttgtgcttttg

SEQ ID NO: 46 - DNA, 1434 bp, Artificial Sequence
  Synthetic
atgggtcagggctcgagcggtgtgcaatctaaccccttagaagattgtttgaaggtagctacaagtccactaggttcatacgccttccatgacaaattgctgtttcaacttaccgatgttaagccttataatttagactacccagtcaacccaatcgctgttacgtatccaggttccactaaagaggttgcacaaattataaagtgcgctaccacttacgataagaaggtccaagccagaagcggaggtcactcttacgctaatttcgctttgggtgacggtgacggtgcaattgttatcgatatgcaaaaatttaagcaattctccatggacacttctacctggcaggctacaattggtcctggtactttgttgggtgatgtctccaagcgtttacacgaaaacggtaacagggtaatcccacatggaacctctccacaaataggtttcggaggccacggtactattggtggtctgggccctttgtctcgtatgtacggtttaaccttggactccatcgaagaagttgaagccgtcttggctaacggtcaaattgttagagctagtaaaactcaaaatgaagatctattttttgctattagaggagccgccgcttcagtcgcagttgtcacagaatttaaggttagaacctatccagagccctctagttctgtgttatattcttacactttacaaggtggttcagttgcttccagagctaacgctttcaagcagtggcaaaaattgacgacagatccatcggtcagcagaaagttcgcttctactttcgttctatccgaagccataaccgtcgtcacgggtactttcttcggtactcaagctgagtttgattccttggacatcacctctaggttgcctgccgacatgatctccaataatacagaagttaagaactggttgggtgtcgttggccattggggtgaatcattggctttgagagccggtggtggtattccagcacacttttactccaagtctttgggtttcaaaaaggatgagatcatggatgatgctactgtggacaagctattcaattatattgacaaagctgataaaggaggtgctgtttggttcgttatttgggaccttgaaggaggtgctatctctgatgttccaaccactgaaacttcttacggtcatagagatgcaatctttttccaacagtcttatgcaattaacttattgggtagagttaaggacgacacccacgaatttttgaacagagttaatagtgtaattatggaatctaacccaggtggttactggggtgcctacccaggttatgtcgatactgctctaggtaattccagcgctaaggcctactggggtatcaacagcgaaagattacaaaccataaaaagttgggtagacgctggtgatgtgtttcacaacccacaatcagttagacccaag

SEQ ID NO: 47 - DNA, 1578 bp, Artificial Sequence
  Synthetic
atgcagccttttacaagccttactaggtcccccttccgttcagcccacgttatcagttgtccagtcgctttggacaatccaccatcggtaccaattataatgggacaaaagccttcctctccattagctacctgcttggataaagtttgtaacggtagatctagttgtgtcggttacccaaacgaccccctattccaaatcaattgggttaagccatataacttggatattcctgtccaaccaattgcagtgactagaccatctaccgctgaggatgttgccggttttgttaagtgtgctgctgaaaacaatgtcaaagtccaagcaaagtctggcggtcattcctacggtaacttcgctatcggtggtactgacggtgccttagttattgatctggtgaattttcaaaacttcagcatggatacaaacacctggcaggctacgttcggtggaggccacaagttgcatgaagttactcaaaaactacacgacaatggtaagagagctatcgcccacggtacctgtccaggtgttggtataggtggacatgctactattggtggtttgggtccatcttctcgtatgtggggctcctgcttggatcacgtagttgaagtcgaagtcgttaccgcagacggtaagatccaaagagctaacgataagcaaaattccgacttgttctttgccttaaaaggtgcaggagctggttttggtgtcattactgagttcgtgatgagaacccatccagaacctggtgacgttgttcaatattcttacgctatcacttttgctaaacacagagacttggttcctgtattcaagcaatggcaagaactgattttcgatccaacacttgatagacgtttctcatctgaatttgtcatgcaagaattaggtgtcgctataacggccactttttacggcacggaggatgaatttaagaagactggtattccagacagaatccccaaaggtaaagtttccgtcgttataaacaattggttgggtgatgtcgcacagaaggctcaagatgcagccttgtggcttagtgatattcaatcagctttcacctctaagtccttggctttcacccataacgacctaatctcggaagacggtatccaaactatgatggactatgttgattcagtcgatagaggcacattaatttggttcttgattttggattctactggaggagctattaatgacgttccaatgaacgctacagcctacagacacagggacaaagtgatgttcttccaaggttacggtgttggtataccaaccctttctggtaagaccaaggattttatgtccggtgttgctgataagatccgtaaggcctctcctaacgaattgagcacttacgctggatacgtagacccaactttggacaatgctcaagaaagatattggggtccaaacttaccagccctagaaagaataaaagctacctgggatcctaaggacttattctcaaacccacagtcagtgaggccaaacgcttccgccaaggatgtcgaacctgccgcatctggtggttccaataattcgggttctaaaggtggagacagt

SEQ ID NO: 48 - DNA, 1419 bp, Artificial Sequence
  Synthetic
atgggatccggtcatagttctggcttggccacttgcttagatgcagtgtgtaatggtcgtcacgcttgtgtagcttaccctgaccacctactgtatcaagcctcttgggtcgatagatacaaccttgacatcccagttcatcccatagctgttaccaggccatcaaacgcagacgatgtcagcggttttgttaaatgtgctgccgctaataacgtcagagttcaggctaagtctggtggtcactcgtatgctaattacggcttgggtggtgaggatggtgaattagttattgacttgagacatttgcaacacttctcaatggatacgaacacttggcaagctaccattggtgccggtcacagattatgggacgttacacataagttgcacgaaaacggtaagagagcagtcagccacggaacttgcccaggtgttggtattggcggtcatgccaccatcggtggtctaggtccatcctctcgtatgtggggatcgtgtttggatcacgtggtcgaagttgaagttgtgactgctgacggttctataagaagagcttccgaaagagaaaacgctgatttgttctttgctttaaaaggtgccggtgctggtttcggtgtgatcaccgaatttgtaatgaagactcaccctgaaccaggatctgttgtcatgaggtacacatactccgttaatttcggtagacatgcagacatggtcgacgtattcgatcaatggcaagctttgatttctgatccaggtctggatagaagatttggaagtgaaattatcatgcacgcattcggcctagtcatttccgctacgttccatggtaccagagatgagtatgaagcttctggtatcccagacagaatccctcgtggtaacgtgtccgttttgttggacaattggttaggtgtcgttggtaatcaggcccaagatgctggattgtgggtttctgaggttagatcgagtttcacttcacgttcattggcttttagaagggaccaacttctatctcgtgatgatattgtcagaatgatggactttttggacagaactgataagggtacgttagtctggtttttgattttcgacgtcacaggtggtgctattggcgacgttagaactgacgcaaccgcctacgctcatagagataagatcatgttctgtcaaggttacgcagttggtataccagctcttaccagaaaaactcgtgtcttcatggacggtttaatttccactatcagggaaaccgccaactctactctaaccacctatcccggatacgtcgatccaagtttgcacgacgctcaagcttcctactggggtcctaacttgccaagattaacagaagttaagactaagtgggatccacaggatgtttttcacaacccacaatctgtaagaccatctggtaaagat

SEQ ID NO: 49 - skipped

SEQ ID NO: 50 - DNA, 1428 bp, Artificial Sequence
  Synthetic
atgggtaacactacatcaatagctgccggccgtgactgcctattgagcgctgtgggtggaaatcacgcacatgttgcttttcaggatcaacttttataccaagctaccgccgtcgaaccctataacttgaatatccctgtaactccagcagctgttacgtacccacaaagtgctgatgaggttgccgctgtcgttaaatgtgccgctgactacggttataaggttcaagctaggtccggtggtcactcgttcggtaactacggtttgggaggtgaagacggtgctattgtcgttgatatgaagcatttcgatcagttttccatggacgaatctacctatactgcaacgatcggtccaggcattactttaggtgatctggataccgccttgtacaacgctggtcacagagctatggctcatggtatctgtccaacaattagaactggtggtcaccttaccattggtggattaggtcctacagctagacaatggggcttggccctggaccacgttgaagaagtggaagtcgtcttggctaactcgtctatagttagagcatctgacacccaaaatcaagaaatcttgttcgctgtaaaaggtgctgctgcctcattcggtattgtgactgaatttaaggttcgtactgaggaagccccaggtttggccgtccaatattctttcacctttaatttaggtactgctgctgaaaaggcaaagctggttaaagactggcaagctttcatcgctcaggaggatcttacttggaagttctactctaacatgaacattattgatggtcaaatcatcttggaaggcatctactttggttctaaggccgaatatgacgctctaggtttggaggaaaaatttccaacctccgaaccaggaaccgtcttggtattgactgactggctaggcatggtgggtcacggtttggaagatgttatattaagattggtcggtaatgccccaacttggttctacgccaagtcccttggatttgcaccaagagcactaattcctgattccgcaattgatgacttcttcgaatacatccataagaacaaccccggtaccgtttcttggttcgttactttgagtttagagggtggtgctataaataaggtcccagaagatgctaccgcttatggtcatagagatgttctattctgggtacaaattttcatgatcaatcctttgggtccagtctcacaaactatttacgactttgcagacggattgtacgatgttttagccaaagctgttccagaaagcgctggtcatgcttacttgggttgtcccgacccaagaatgccaaacgctcaacaagcttactggaggaacaatttgcctagattagaagaacttaagggtgatttggacccaaaagatatattccacaacccacaaggtgtcatggttgtttcc

SEQ ID NO: 51 - DNA, 1431 bp, Artificial Sequence
  Synthetic
atgggtcaatcccccagttcacttttagccacttgcctaaataccgtttgtgacggcagaacagattgtgtagcataccctaacaacccattgtatcagatcagctgggtcaaccgttacaatctggatttgccagttactcctattgctgtcaccagaccacaaacggttcaagacgtgtctgcttttgttaaatgtgctgccactaacaatataaaggtccaaccaaagtctggtggacactcttacgctaactatggtggtgaagacggtgctttagttattgatttgttgaagttgcaagatttctccatggacgccaaaacctggcaggctactatcggtggtggtacaaagttggctgatgtcaccaagagactgcatgataacggtaaaagggcaatttctcacggtacttgtccaggcgttggtatcggtggtcatgctaccatcggtggcttgggacctacttcgagaatgtggggttcctgcttagaccacgtcgtggaggctgaagttgtgactgccgatggtagtattaagagagcctctgaaacagaaaatcgtgacttgttcttcgctcttaaaggtgcaggagcaggttttggtgttgtcacgaagtttgttatgaagacccacccagaaccaggtagcatggtacaatactcctattcactatctttcggtaaacatactgatatggtaccagtttttaagcaatggcaagatttagtcagtgaccccaatttggacagaagattcggcactgaatttgttgctcatgagttgggtgctattatcaccgctactttctacggtacagaagctgaatgggatgctagcggcatcccacaaagaatcccaaagggtaagatatccgtcattattgatgattggctagccgttatttcccagcaagcagaggacgctgccctatatttgtctgacattcactccgctttcaccgtgcgttctttggccttcaccgctgaagaaacattgtctgaacaaactatcactagagttatgaagtacatcgacgatacgaacagaggtaccttgttatggtttttaatattcgacgcaacgggtggtgctataagcgatattcccatgaatgctactgcctactcccacagggacaagatcatgtactgtcaaggctacggtattggtctaccagtcttaaaccaacatactaaagatttccttacgggtcttaccgacactatccaggcttctatgagacaaaacttgactacctacccaggttatgttgatccttcattggctaatccacaacaatcttattggggcccaaaccttgcaatgttggaatcaattaagaccacgtatgacccaaacgatttgttccataacccacaatccgttagaccaggtaataaaaaggcttctatgactcaagagttt

SEQ ID NO: 52 - DNA, 1419 bp, Artificial Sequence
  Synthetic
atgagtaataacacccttattcaagcttgcctgttagccgctctaggattcaactctactagagtaaaatttcctgaagcaggtacaatcgacgccgatccatataacttggacttgcccatagttccagctgctattactgtcccacgttccacggctcaggtggccaatgtcgttaagtgtgctgcatcgaacggttacaaggttcaatcaagatctggcggtcacagctacggtaatcatggtttgggcggtaccgatggtgctatcgttgtggatttaaaagacttaaagcaattctctatggatgagtccacttacattgcatctatcggttcaggtatgttgttggacgaagtcacccacaagttgtatgataacggtaagagagccatggctcatggagtttgtccacaagtcggtgtgggtggtcactttacaattggtggtttgggacctactgctagacagtggggttcctctcttgaccatgttgaagaagttgaggtcgttttggccaattctagtatagttagggcttccaacactcaaaaccaagacgtcctattcgctatcaaaggtgctgctgcttctttcggtattgtgaccgaatttaaggttagaaccgaaccagccccagatgtcgcagttcaatacacttatgaattaattttgggtaatatcacagaaagagctaggattatggtcgattggcaggacttcatatccgaccccgatctttctcgtaaatttgcatcgattatgatcgtcttcgaacacggtatgttgctgtccggcgatttctacggttcgaagaaggaatttgatgacctgggtttggctgacagattcccaattagaaagccaggtaacgttgctattttgactgattggttaggaatgacaggtcatgctgttgaggaattggctttaggtatcgtaggtggcatccctttgcacttttatgccaagagtatggctttcacccgtgacagtttaatgtctccatctacgttcgaaaaattgtttgattaccttgataacactgataagggcaccttgctatggctaatttacttcgatttgcaatcaggcgccacttcagacgtaccaaataattctacagcatacgctcacagagacactttgtactggctgcaatcctacgtagttaacttggtgggtccagtcagtaacaccaccacagcttttcttgaaggtataaataacttgatcgcccaagacgtaccttccgccaacactagagcttatccaggttacgttgatccattaatgcccaacccacaatggcgttattggggttcgaatttaccaaagttggaaaagataaaagcagctattgaccctaatgacgtcttccataacccacaatcagtcaagcctagaaaacaagcaccc

SEQ ID NO: 53 - DNA, 1512 bp, Artificial Sequence
  Synthetic
atgggacagagccaagcattgtcagcctttaaaaaggacctagctgctgctcttaataacgatgatgaattgttcgctctgccagacgagcctttctacattaaggaccacgtcaagcgttataacttagatatcgaaaccacacccttagccgtaacgtacccaaaaactaccgcacaagtttcttccatagtgaagttggctaaggataacaatttgaaagttcaagctaagtgcggtggtcattcttatgccaatttttgtgatccaaacggtggcatcattgtcgaccttaaacacttccaaaagtttgaaattgacgaaaacacttggagatgtagggtcggtggtggtactttgctaggtgatttgactaagagaatgttcgaaccacataagagagctatggctcacggtacctgtccaacagttggtatcggaggtcatgcaaccatcggtggcttgggtcctagttcgagattatggggtgctgccttagaccacgtagaggaagtcgaaatggttgttgctaacggtgacgttattagagccaacgaaaacgaaaattccgatatattctgggctgtcaaaggcgctggtgcctcttttggtgttattactgaatttgttgtaagaactgagccagctccaggtagattggttcaatactcttattccttcacaaccggttcatggaaggacatggccaagactttcaaggcttggcaaacctacgtcagccaacctgatttgacgcgtagtttcgcaagtactgctacgatcaccgaactgggtttgactatttctgtcacatacttcggcactgacgaagaatttgataaaataaatttcgctaaaaatttcccaggtaaccagaccccaaagaccatcgtttttgatgattacttgggtgctgtgggacattgggccgaagatgttgctttagaaattatctctcctttgcccgcccactcctatacaaagactttgacttttaaccactgcaaccaaattccagactctgtgattgatagaatgttcaaatacttcgaggaagtttcgaagggtacgttagtttggtttgccatcttcgatttggctggtggtagagtcaatgacatcccacaagacgctaccgcatatgctcatagagatgctttgttctacttacaatcctacgctgtgaacccatttggtcccgtttctaataaaagtaagcaatttctgcaaggccttaacaaggtcatccgtgatggaatggctgaagctggtgaaaatacagacttgggtgcatatgccggctacgttgatctggaattaggtgctggtgctcagaaggcctactggagaactaacttgccacgtttggagtctattaagctaaagtgggacccagaggatgtattccacaatcctcaatccgtcagaccaggtggtaacgacgttatttctaccccaaaggtagtctacaaaaaggctggtttcctagctaggttaaaaggttgtttcaga

SEQ ID NO: 54 - DNA, 1422 bp, Artificial Sequence
  Synthetic
atgggtaatacaaccagcattgctggaagggattgcctagtctctgcattgggcggtaacgccgacttagttgcttttcaaaaccagttgctttaccaaactactgctgtgcacgagtataatctgaacatacccgttacgcctgccgctatcacctacccagaaactgctgaacaaattgctgctgtcgttaaatgtgcctccgaatacgattataaggtacaagccagatcaggtggtcattctttcggtaattacggtttgggtggaaccgacggtgctgttgtcgttgatatgaagcacttcaaccaatttagtatggacgatcaaacttatgaagctgttttaggtccaggtactaccttgggcgacgtcgatacagaattgtacaacaacggtaagcgtgctatggcacatggtatctgtccaacgatttcaaccggtggtcacttcactatgggtggcttgggtccaaccgccagacaatggggtttagctcttgaccatgtcgaagaagtcgaggttatccttgctaattcttccatcgtaagagcctcgaacacccagaatcaagaagttttctttgcagttaaaggagctgccgctagtttcggtattgtcacagagtttaaggtcagaactcaaccagcacctggtttggctgttcagtattcttacaccttcaacttgggttcttccgctaagaaagctcaattcgttaaggattggcaaagctttatatccgctaaaaatctaactagacaattttacactaacatggtaatcttcgacggtgatattattttggaaggcttattcttcggctctaaggaacaatacgaagcactgggtttggaagaacgttttgttccaaagaatccaggtaacatcttggttctaacagactggttgggtatggtgggtcacgccttggaagacactatattgagacttgtcggtaacactcctacctggttttacgcaaagagcttgggtttcactccagatacgttaattccttctgctggtattgatgaatttttcgaatatatcgaaaacaacaaggctggcacatccacctggtttgtcaccttatctttagaaggtggtgccattaatgacgtaccagctgatgctacggcatacggtcacagagatgtgttgttctgggttcagattttcatggtcagtccaactggaccagtttcgtctaccacttatgacttcgctgatggtctgtacaacgtcttgaccaaagctgtgccagagagtgagggtcatgcttacttgggttgtcccgatccaaaaatggccaatgctcaacaaaagtattggagacaaaaccttcctagactggaagaattgaaggctatcttagatccaaaggacacttttcataacccacaaggaattttacccgcc

SEQ ID NO: 55 - DNA, 1428 bp, Artificial Sequence
  Synthetic
atgggtaataccacatccatcgccgctggacgtgattgcctattgtcggctgttggcggtaaccacgcacatgtcgccttccaggaccaattattgtatcaagctactgctgtggagccatacaaccttaacatacctgttactccagctgctgtcacgtacccccaaagcgcagacgaaattgccgctgtagttaagtgtgctgctgaatacggttataaagtccaagcaagatcaggtggtcactcttttggcaattacggtctgggtggtgaagatggtgccattgttgttgaaatgaagcatttcaaccaattttctatggacgaaagtacctatactgctaccatcggcccaggtattactcttggtgatttggatacaggtttgtacaacgccggtcacagggcaatggctcatggtatctgtccaactattagaaccggaggtcacttgactatgggtggtttaggtccaacagctagacagtggggattagctttggaccatgttgaagaggtcgaagtggttttggcaaattcctctattgtcagagctagcgacacccaaaatcaagatatattcttcgctgttaagggtgccgctgcctcttttggtatcgtaactgaatttaaagtcagaaccgaagaagctcctggattagctgtccaatactccttcactttcaacttgggtaccgccgccgaaaaggctaaacttgttaaggactggcaagctttcattgctcaagaggatttgacctggaagttttactccaacatgaacatcttcgatggtcaaataatcttagaaggtatttactttggttctaaggaagaatatgatgcattgggtttagaagagagattcccaacctctgaacctggtactgttctggtgttgacagactggttgggtatggttggacacggcctagaggatgtcattttgaggttagtgggtaatactccaacttggttttatgccaaatcactaggtttcgccccacgtgccttgatcccagacagtgctattgatgatttcttttcttatatacacgaaaacaacccaggtactgtttcttggttcgtaacgcttagcttggaaggtggcgctatcaacaaggttcccgaagacgctaccgcttacggtcacagagatgtgttgttttgggtacaaattttcatgattaatcctttaggtccagtttcgcagactacctacggtttcgcagacggattgtacgacgtcctagctaaggctgtcccagaatcagctggtcatgcatacctgggttgtcccgacccacgtatgccaaacgcccaacaagcttattggagatccaacttgccaagattggaagaattaaaaggtgaattggatccaaaggatatctttcataatccacagggtgttatggttgtttct

SEQ ID NO: 56 - DNA, 1419 bp, Artificial Sequence
  Synthetic
atgggaaataccactagcattgcaggtcgtgactgcctaatatctgccttaggtggtaactcagctcttgctgctttccctaaccaactgttgtggacggccgatgtacacgaatataatttaaacttgccagttacaccagctgctatcacttaccccgagactgccgaacagattgcaggcatcgtcaagtgtgcttccgactacgattacaaagtgcaagctaggtctggtggtcatagttttggtaattatggtttgggcggaaccgacggtgccgtcgttgttgatatgaagcacttcaaccaattttcaatggacgatcaaacctacgaagctgttattggtccaggtacaactttgaacgatgttgatatagaattatacaataacggtaagagagccatggctcatggcgtctgtcctactatcaaaaccggaggtcacttcactattggtggtttgggtccaaccgctagacaatggggtcttgctttggaccacgtagaagaggtcgaagtcgttttggctaactcttccatcgttagagcaagtaatacccaaaaccaagatgtcttgttcgccgttaagggtgctgccgctgactttggaattgtaaccgaatttaaggttagaactgaaccagctccaggtttggccgttcagtattcgtatacgttcaacctaggttctactgctgaaaaagctcaattcgtgaaggactggcaatctttcatttccgctaaaaatttaaccagacaattttacaacaatatggtcatcttcgatggtgatatcattctggagggtttgttctttggtagcaaggaacaatacgatgccctaggtttggaagaccatttcgcacccaagaacccaggtaacatcctggttttaaccgactggcttggcatggtcggccacgctttggaagatacaatacttaagttggtcggtaatactccaacttggttttatgccaagtctttgggtttcagacaagatactttgattccttccgctggtattgatgaatttttcgaatacatagacaaccacacggctggtactccagcttggttcgttacattatcattggagggtggtgccatcaatgacgtggccgaagatgctactgcatacgcccatcgtgatgttttattctgggttcagttgtttatggtcaacccacttggtccaatctctgaaacaacctacgaatttacggatggtttgtatgacgttctagctagagctgttcctgagtctgttggtcatgcctacttgggatgtccagatccacgtatggaaaacgcacctcagaagtactggagaactaatttacctagattgcaagaactgaaggaagaattggacccaaaaaatacattccaccatccacaaggtgttattccagct

SEQ ID NO: 57 - DNA, 1419 bp, Artificial Sequence
  Synthetic
atgggtaataccacatctattgccggcagagactgcctaatcagcgctttaggtggagattccgcactggctgtcttcccaaaccagcttttgtggactgctgatgtgcacgaatacaacttaaatcttcctgtaactccagccgctataacctatcccgagacagctgaacaaattgccggtatcgttaaatgtgcttcagactacgattataaggttcaagcacgtagtggtggtcattcctttggcaactacggtttgggtggtactgacggtgctgttgtcgtcgacatgaagcacttcaatcaattttctatggatgatcaaacctacgaagcagttattggtccaggtactaccttgaacgacgttgacatcgaattgtacaacaatggaaagagagctatggctcatggtgtatgtccaaccataaaaactggtggtcatttcacgattggtggtttgggtcctacggccagacaatggggcttggctttagatcacgttgaagaagttgaggtcgtcttggccaactcttcgatcgtcagggcttctaatactcaaaaccaagatgtctttttcgctgttaagggcgccgcagctgacttcggtattgtgactgaatttaaggttagaacagaaccagctccaggattggccgtgcagtatagctatactttcaaccttggtagtaccgctgaaaaagctcaattcgttaaggattggcaaagctttatctccgccaagaacttgacgagacaattctacaataatatggtcattttcgacggtgatattatcttagagggtttgttctttggttcgaaggaacaatacgacgctttgggtttggaagaccactttgcaccaaaaaacccaggtaacattctagttctaaccgattggttaggtatggtaggacacgctttagaagatactatcttgaagctagttggtaatacccccacttggttctatgcaaagtctttgggttttagacaggacacactgatcccttctgctggaattgatgaatttttcgaatacattgctaaccacaccaccggtactcctgtttggctggttactttgtcattagaaggtggtgccattaatgatgtagctgaggatgcaacagcttacgctcatagagatgtcctattttgggttcaattgttcatggttaacccattgggtcctatttctgaaacaacttatgaatttacagacggattgtacgacgtcttggcccgtgctgtcccagagtccgtcggtcatgcctacttaggctgtccagacccaagaatggaagatgctccacaaaagtactggcgtaccaacttgccaagattgcaagaattgaaggaagaattagacccaaaaaacacgttccaccatccacaaggtgttatacccgcc

SEQ ID NO: 58 - DNA, 1446 bp, Artificial Sequence
  Synthetic
atgggtaataaagcaagtaccacaacgataatcaccactgctgtacacaagtgccttctgtcggccgtgaacggcaactcagctcaggtttccgtccaaaacgacttattgtacggtgttaccgctgttcatgaatataatttgaactttccaatgactcccgctgccgtcactttccctgagacttccgaacaagttgctgcattggtcaagtgtgctgccgaatacaagtataaagtgcaagctaggagcggaggtcactctttcggtaaccatggtctaggtggtgctgatggagctattgttgtcgatatgaagcactttcaacaattctctatggacaatgaaacccacgttgccacaattggcccaggtttgagtctaggtgacatcgatacacttttgtacaacgctggtggtagagccatgagccatggtatttgtccagaaatacgtgccggaggtcacttaactatcggtggtttgggtttgacttctcgtcaatggggtatgtctttagaccatatcgaagaagtcgaggtagttttgccaaattcctcgatcgttagagcttctgaaaccgaaaatgctgatctattattcgctgttaagggcgcagctgcatcttttggtgttgtcactgaatttaaggtaagaacgcaacttgcacctaaagaagctattcagtactcatacagtttcaaattgggttccgctgcccaaagagctagattgttcgctgattggcaagacttggcattaaggagagatttgtctcgtaagttcacatccgatttcatttgtttgcaagactctgtcattgtgaagggtgtgtttttcggttccaaaaaggaatataacgccctaagaattgaacatcacttaccaggctctgacagttctaaggttttggtcttagatgactggttgggtattgttacccacgttgtcgatgatctggctgttagattaggtggttccatgtcaacttacttttatgccaagtcacttggttttaccagagatactttgatgccaccatcgacgatcacctctttattcacttacttggacaaagctaagaaaggcacaataacttggttcgtcaccttcagcttggtcggtggtgctatcaatgattaccctaagaacgccacggcttatccacacagagatgttatctactggatgcaatcttttgctattaacgctctgggtcctgttttgaactccacttacgacttcttggacggcatcaatgagctagtcgcacgtgatttaccaggttgtgccggacacgcttatttaggttgcccagatcccagaatggagggtgctgaaagagcctattggggttcaaacttaggtagacttgaagacatgaaaggtgtctttgacccagttgacgttttctggaatccacaaggcgtcggtgtccctgttgct

SEQ ID NO: 59 - DNA, 1419 bp, Artificial Sequence
  Synthetic
atgggtaacaccacttccatagcaggccgtgattgcctaattagcgctcttggtggtaatagtgctctggccgtgttccccaaccagttattgtggacagctgacgtccatgaatacaatttgaacttacctgttactccagcagctatcacgtatccagagactgctgaacaaatcgctggaattgttaaatgtgcctctgattacgactataaggttcaagctaggtctggtggtcactcctttggtaactacggtttgggcggtaccgacggtgccgtcgtagtcgatatgaagcacttcactcaattttctatggatgaccaaacctacgaagcagttataggtccaggaacaactttgaatgacgttgatattgaattgtataacaacggtaaaagagctatggctcatggtgtttgtccaaccatcaagacaggtggtcacttcactattggtggtttaggtccaaccgccagacaatggggattggctttagaccacgtcgaggaagttgaagtcgttttggctaactcatcgatcgtcagagccagcaatacccaaaatcaggatgtctttttcgctgtaaagggtgcagctgccgacttcggcatcgttactgaatttaaagttagaaccgaacctgctccaggtttggccgtgcaatactcgtatacattcaacctaggttccacggctgagaaggctcaattcgtcaaggattggcaatcttttattagtgcaaagaacttgactagacaattctacaacaacatggttattttcgacggtgatattatcttggaaggtttgttctttggctcaaaagaacagtacgatgctcttggtttggaagatcatttcgctccaaagaatccaggcaacatcttagttttgactgactggctgggtatggtgggtcacgctctggaagatacgattttgaagcttgtcggtaatacccccacctggttctatgctaagtctctaggttttagacaagataccctgattcctagtgctggcatcgatgagttctttgaatacatcgacaatcacactgccggaactccagcttggttcgtaactttatccttggaaggaggtgccataaatgatgttgccgaagacgctactgcctatgctcatagagatgttttattttgggttcaattgtttatggtcaaccctttgggtccaatatctgaaactacatacgaatttactgatggtttatacgacgtattggccagagcagtaccagaatccgttggtcatgcttaccttggttgtccagacccacgtatggaaaatgcacctcaaaagtactggaggactaacttgcccagacttcaggaattgaaagaagagctagacccaaagaacaccttccaccatccacaaggtgtcattccagct

SEQ ID NO: 60 - DNA, 1422 bp, Artificial Sequence
  Synthetic
atgggcaacaccacatctatcgctggtagggactgcttggtatcagccctgggtggtaatgctggtcttgttgcatttcaaaaccagcctttgtatcaaactactgctgtgcacgaatacaatttaaacataccagttaccccagccgctattacgtacccagagactgctgaacaaattgccgctgtcgtcaagtgtgcatcccaatacgattataaagtccaagctagaagtggaggtcatagcttcggtaattacggtctaggcggtacagatggtgctgttgttgttgacatgaagtacttcaaccaattttctatggacgatcagacctacgaagctgtcatcggtcctggtacaaccttgggagatgtcgacgtcgaattatataacaacggtaagcgtgccatggctcacggtgtttgtccaactatttcgactggaggtcatttcactatgggtggtttgggtcccaccgccagacaatggggcttagccttggaccacgttgaagaagtagaagtagttttagcaaactcctctatcgtgagagctagcaatacgcaaaatcaagaggttttctttgctgttaaaggcgctgctgcctctttcggtattgtcactgaatttaaggttagaactcagccagctccaggtatagcagtccaatattcctacaccttcaacttgggttcgtctgctgagaaggctcaattcatcaaagattggcaatcatttgtctccgctaagaacttgaccagacaattctacaccaatatggttattttcgatggtgatattatcctagaaggtttgtttttcggttccaaggaacagtatgaagcattgcgtcttgaagagagatttgtgccaaaaaacccaggtaacatcttggttctaactgactggctaggtatggtcggacatgccttggaagacacaatcttgagacttgttggtaatactcctacttggttttacgctaagtctctgggtttcacaccagatacgttgattccatcttctggaattgatgaatttttcaagtacatagaaaacaataaggccggcacctccacttggtttgttacattatcattggaaggcggtgctatcaacgatgtacctgctgacgccaccgcttatggtcacagagatgttttattctgggtccaaattttcatggtttcaccaactggtccagtttcttctaccacctatgacttcgctgatggtttatacaatgtcttgactaaagctgtacccgagagtgaaggccatgcttacttgggttgtccagaccctaagatggctaatgcacaacaaaagtactggagacaaaacctaccaagacttgaagaattgaaagaaaccctagaccccaaggatacttttcacaacccacaaggtatcctaccagcc

SEQ ID NO: 61 - DNA, 1449 bp, Artificial Sequence
  Synthetic
atgaatccctcaattccatcctctagtatgggcaacactacctcgatagccggtagagactgtctagtgtctgcacttggaggtaacgctggtttagttgctttccaaaaccagccactgtatcaaacaactgctgtacacgaatacaatttgaatacccctgttacgccagccgctatcacttacccagagacagctgaacatattgctgccgtcgttaaatgcgcaagccaatatgattacaaggtccaagctcgttctggtggtcactcctttggtaactacggtttgggtggtaccgatggagctgtcgttgttgacatgaagtatttcaaccaattttctatggatgaccaaacctacgaagctgttatcggtcctggtactactttgggtgatgtggatgtagaattgtataacaacggtaaaagagccatggctcatggtgtctgtccaactatttccaccggcggtcacttcacaatgggcggtttaggtccaactgctagacaatggggtttggctcttgaccacgtcgaagaagttgaggtggttctagcaaatagttctatcgtcagggcctcgaatactcagaatcaagaagttttctttgcagtaaagggagctgctgcttcttttggtatcgttaccgaatttaaggtcagaacgcaaccagctccaggaattgctgttcaatactcatacaccttcaacttgggttccagcgccgaaaaggctcagttcattaaggactggcaatctttcgtgtccgctaaaaacttaaccagacaattctacacaaacatggttatatttgacggtgatattatcttggaaggtctatttttcggttccaaagagcaatatgaagctttgggtttggaagaaagattcgtcccaaagaaccctggcaatatcctagttttaacggattggttgggtatggtcggacatgccttagaggatacaatattgagattggttggtaacactcccacctggttctacgccaagtcccttggttttactccagacacattgattccttcttctggtatcgatgaatttttcgaatatattgaaaacaataaggcaggtacttctacctggtttgtcaccctttcattggaaggtggtgccattaacgacgtcccagctgatgctactgcatacggtcatcgtgacgtgctattctgggttcagatatttatggtaagtcccactggcccagtaagttccacgacttacgatttcgctgacggtttatataatgttctgactaaagctgtgccagaatctgagggtcacgcctacttaggatgtccagatccaaagatggctaatgcacaacaaaaatactggagacaaaacttgccaagattggaagaactaaaggaaactttggacccaaaagataccttccataatcctcaaggcatccttcccgcc

SEQ ID NO: 62 - DNA, 1848 bp, Artificial Sequence
  Synthetic
atgggtaatactacctcaatagccggcagagattgcctagtctccgctttgggaggtaacgcaggtctggtggcttttcaaaaccagcctttgtatcaaacgacagctgtacacgaatacaatcttaacattcccgtcactccagccgctatcacctacccagagactgctgaacaaatcgccgcagttgttaaatgtgcttcgcaatacgactataaggttcaagctaggtctggtggtcattccttcggtaactacggattaggcggtacagacggtgccgtcgttgttgatttgaagtacttcaatcagttttctatggatgaccaaacctatgaagctgtcattggtccaggtactaccttgggtgatgtagacgttgaattatataacaacggtaagcgtgctatggcccacggtgtatgtccaactattagcacgggtggtcatttcactatgggtggtcttggacctacggctagacaatggggtttagccttggatcacgtcgaagaagttgaggtcgttttggctaactctagtatcgttagagctagcaatacccaaaatcaagaagtgtttttcgctgttaaaggcgcagccgcttcgttcggtattgtcactgaatttaaggttagaactcaaccagctccaggtattgctgttcaatactcttacaccttcaatttgggctcttccgccgagaaggcacagtttataaaagactggcaatcattcgtttctgctaagaacttgacaagacaattctataccaacatggtcatctttgacggtgatattatcctagaaggtctgtttttcggtagtaaggaacaatacgaagctttgcgtttagaagaaagattcgtgcccaagaaccctggtaacattttggttttaactgattggctaggtatggtcggtcacgctttggaggacacaatcctaagattggttggaaataccccaacttggttctacgctaagtccttgggatttactccagatactttgataccatcttccggtatcgacgaatttttcgaatatattgaaaacaataaagccggtacctctacatggttcgtaaccctttctcttgagggtggagccatcaacgacgttccagctgatgctactgcatacggtcatagagatgtcttgttttgggtacagattttcatggtcagccctacaggtccagtttcctctacgacctatgactttgctgatggtttatacaacgttttgactaaggtggttccagaatccgaaggccacgcttacttaggttgtccagacccaaaaatggccaatgctcaacaaaagtattggaggcaaaatttgccaagactagaagaactaaaagaaacactggaccctaaggatacttttcacaatccacaaggcgtcttgatcaccgaggttggttccgccacggacttctggaacttagttgaagctattatcttaatctctcagttgcatgaatcagtcggccaaacatacaacatggtgcccgagatgggtgaacaacctgttagagaaatgactaagatgttccgtatgttggaaaagactattcaagtcagcttggaaggtcttccatacgaggaatggttgaacagactgcaagtggaaaacgatgatgatccactgaggccactgttgccaatgtttgaagaaaaagtctacgacggtagatgccaatgggaaatgtacgagaacatgcctatttcggacaccgaaaacttgagacaatacttgcaagatgttcctgaattagcaacttgtccattcttggatcaagatatatttaagaagttcctttcctctcttggtttggca

SEQ ID NO: 63 - skipped

SEQ ID NO: 64 - DNA, 1419 bp, Artificial Sequence
  Synthetic
atgggcaatacaacttcgatagctggtagagactgccttatttcagcactgggtggaaacagcgccttagctgcttttcccaacgagctattgtggacggccgatgtccatgaatacaatttgaacttgccagtgactcctgctgctatcacctatccagaaaccgctgaacaaattgcaggagtagttaaatgtgcctctgactacgattacaaggtccaggctcgttccggtggtcacagtttcggtaactatggtttaggtggtgcagatggtgctgttgtcgttgacatgaagcacttcactcaattttctatggacgatgaaacctacgaagctgttatcggtccaggcactacattgaatgatgttgacattgaattatataacaacggtaagagagccatggctcatggtgtgtgtcctaccatcaaaacaggtggtcacttcactattggcggtttgggtccaactgctagacaatggggtttagctttggatcacgtcgaggaagtcgaagttgttttggccaactcttccattgtcagggcatctaatacccaaaaccaagacgtgtttttcgctgttaagggcgccgctgctaacttcggaatcgttaccgaatttaaggtcagaactgaaccagcaccaggtttggccgtccagtactcgtatactttcaatttgggtagtaccgccgaaaaagctcaatttgttaaggactggcaatctttcatttccgctaagaatcttactagacaattttacaataacatggtaatcttcgatggtgatatcattttggaaggtttgttctttggttccaaagaacaatacgatgctctgggtcttgaagatcatttcgctccaaagaaccctggtaacatattggtcctaaccgactggctaggtatggttggtcatgccttagaagacaccatcttgaagcttgttggtaatacaccaacttggttctatgcaaaatctttgggctttcgtcaagatactctgatcccatcagctggcattgacgaatttttcgagtacatcgctaaccacaccgctggtactccagcctggtttgtaacgttgtctttagagggtggtgctattaacgatatcgccgaagatgctacggcttacgcccatagagatgttctattctgggtccaactgttcatggtcaaccctttgggtccaataagcgacacaacttacgaatttactgatggattatatgacgtattggcaagagcagttcccgaatccgttggtcacgcttacttaggttgtccagatccaagaatggaagatgctcaacaaaagtactggagaaccaacctgcctcgtttgcaagagcttaaagaagaattggacccaaagaatactttccatcacccacagggtgtcatgccagct

SEQ ID NO: 65 - DNA, 1428 bp, Artificial Sequence
  Synthetic
atgggtaatacgacatcaatcgcagccggaagagactgccttctgtcggctgtcggtggcaaccacgctcatgtagcctttcaggatcaattattgtaccaagctactgctgtggagccatataacctaaacatacctgttacccccgccgctgttacttacccacaatctgctgaagaaattgcagctgtcgttcaatgtgcttccgaatatggttacaaggttcaagctcgtagcggtggtcactccttcggtaattacggtttgggcggtgaagatggtgccatcgtcgttgaaatgaaacatttcaatcaattttctatggacgaatctaccaacattgctactattggtccaggtatcaccttgggtgacttggatactgctttatacaacgccggatatagagcaatggctcacggtatatgtccaacaatcagaacaggtggacatttgaccatgggtggtctaggtcctactgccaggcagtggggcttggccttggatcacgttgaggaagtcgaagttgtgttagctaactcttccattgttagagcttcagatactcaaaatcaagacattttcttcgctgtcaagggtgctgccgctagttttggtattgttaccgaatttaaggtcagaactgaacaagctccaggtcttgccgtacaatattctttcactttcaacttacagaccccagcagaaaaagcaaagttggtaaaagactggcaagctttcatcgcccaagaggatttaacatggaagttttactcaaatatgaatattttcgatggtcaaatcattctggaaggaatctacttcggttccaaggctgaatatgacgctctaggtttggagaagagatttcccacttctgaaccaggtaccgtcttggtcttgacagattggctaggtatggtcggtcacggcttagaagatgttatattgcgtttagttggtaacaccccaacttggttttacgccaaaagtttgggcttcacgccaagagctttgatcccagactctgctattgatgactttttcaactatatccacaagaataaccctggtactgttagttggttcgttactttgtctcttgaaggtggtgctataaataaagtcccagaagacgctaccgcctacggtcatagagatgtattgttttgggttcagatatttatgattaacccattaggccccgtcagccaaactacatacggtttcgctgacggtttgtacgatgttttggctaaggcagttccagagtccgcaggtcatgcttacttgggctgtcctgacccaaggatgccaaacgcccaacaagcatactggagatccaacctacctagattggaagaactgaagggtgaattggatccaaaagacgtttttcataaccctcaaggtgtaatggtcgtcagc

SEQ ID NO: 66 - DNA, 1419 bp, Artificial Sequence
  Synthetic
atgggtaacacgacctctatcgctgccggacgtgactgtctgatttcggcagtcggtgctgctaatgttgccttccaggatcaattattgtaccaagctacagctgtacaaccttataacctaaacataccagttactccagccgctgttacctacccacaaagcgcagacgaaattgctgccgtggtcaagtgcgcttcagagtatggctacaaagttcaagctaggtccggtggtcactcctttggtaattacggtcttggtggccaagatggtgccatcgttattgaaatgaagcatttctctcagttttctatggatgaatcaaccttcatcgctactataggtcccggtattactttgggtgacttggatactgacttgtacaacgccggacacagagctatggctcatggtatctgtccaactattagaacgggtggtcacttaacagtcggtggactaggtcctaccgctagacaatggggtttggcattggatcacgtagaagaagtcgaggtggttttagccaacagctccattgtcagagcttctgacactcaaaatcaagatttgttctttgctattaagggtgcagctgcctctttcggaatcgttaccgaatttaaagtcagaactgaacaagctccaggtatggctgtccaatatagttacactttccatctgggcacatccgcagaaaaggctaagttcgttaaagattggcaggcattcatcgctcaagagaacttaacttggaagttttataccaatttggttattttcgacgatcaaatcatactagaaggtatctactttggtacgaaggaagaatacgatagtttaggtttggaacaacgtttcccacctacggacgccggcactgttttgattttaaccgattggctagctatgatcggtcatggtttggaggacaccatcttgaagctagtcggtgatacaccaacctggttttacgccaagtctttaggttttaccccaagagctttgattcccgacagtgctatcgacgaatttttcgattatatccacgaaaataacccaggaactttggcatggttcgttactttgtctttggaaggtggtgccattaacgctgtccctgaagatgctacagcttacggtcatagagatgtgcttttttggttccaacttttcgtgattaacccattgggtccaatttcccaaaccacatacggatttgctgacggtctttatgatgttttggcccaagctgtcccagaatctgtgagccacgcatatatgggttgtccagaccctagattgccaaatgcccaatacgcttattggcgttccaatttaccaaagttggaggaattaaaaggtatattagacccagaagacatctttcacaacccacagggtgttgttccttca

SEQ ID NO: 67 - DNA, 1488 bp, Artificial Sequence
  Synthetic
atgttatcaacaatggccttctcttttgttttgagaattttgtcccctctattcttgatactacagcttagcacggctgcttcgaccagtactttgcgtcaatgcttgctgaccgcagtccaaaacgatccaactttagtagctgtggacggtgatttgttgtatcaaactttagccgttcaagtttacaatcttaactggccagtcacacccgctgctgttgcatttccaaaatctacccaacaagttgcttctatcgtaaattgtgccgcttccctaggctacaaggtccaagccaagtctggaggtcactcctacggtaactatggtctgggtggtactaacggtgctattagcatcaacttaaagaatatgaaatcattctctatgaattacaccaactaccaggctacagtcggtgccggtatgttgaatggcgaattggacgagtatttacataacgctggtggtagggccgttgctcacggaacctctccacaaattggtgtcggtggtcatgctactatcggtggattgggtccatctgcaagacaatacggtatggaacttgaccacgttttggaagctgaagttgttctggctaacggcacggtagtcagagcaagttcaactcaaaactcagatttgttgttcgccattaagggtgctggtgccagctttggtgttgtcactgagttcgtctttagaacagaacctgaaccaggtagtgctgtgcagtataccttcacttttggtttaggctccacgtctgctagagcagatttgttcaagaaatggcaatccttcatatcccaaccagacttgactcgtaagtttgcctctatctgtacgctattggatcatgtacttgtaattagcggtacctttttcggtactaaggaagaatacgacgctttgggacttgaagatcaattccccggtcacactaattcgaccgttatcgtgtttaccgattggttaggcttggttgctcaatgggctgagcaatctatcttggacttgactggcgacattccagctgatttctacgccagatgtctgtcctttaccgaaaaaaccctgattccttctaacggtgtcgaccagttattcgaatatttggatagtgcagacactggtgctttattatggttcgtcattttcgacttggaaggtggtgctattaacgatgttccaatggatgctactggttacgcacacagagataccttgttttggctacaatcatacgctatcacattgggttctgtttccgaaaccacttatgatttcttagattctgttaacgaaatcataagaaataatacccctggtttgggtaatggtgtttaccctggttacgtcgacccaagattagaaaacgctagagaagcttattggggttctaatttgccacgtttgatgcaaataaagtctttgtatgacccaacagacttgtttcataacccacaaggtgtactaccagcc

SEQ ID NO: 68 - DNA, 1419 bp, Artificial Sequence
  Synthetic
atgggcaataccacatccattgccggacgtgattgcctgatcagtgcattgggtggtaactcggctttagctgtctttcctaacgaattgctatggacggctgacgtgcatgagtataatttgaaccttcccgttactccagctgccataacttacccagaaaccgctgctcagattgcaggagttgtcaagtgtgccagcgattacgactataaagttcaagctagatcaggtggtcactctttcggtaactacggtttaggtggtgcagatggagctgtagttgttgacatgaagcacttcactcaattttctatggatgacgaaacttacgaagctgtcatcggtccaggtaccacattgaatgacgttgatattgaattgtacaacaatggtaaaagggccatggctcatggtgtctgtcctaccatcaagactggtggccacttcaccattggtggtttaggcccaactgccagacaatggggtctggctttagatcatgttgaagaggtagaagtcgtgttggctaactcttccatagtcagagcctctaatacacaaaaccaagatgtcttctttgctgttaagggtgcagctgcaaacttcggtattgttaccgaatttaaggtgagaactgaaccagctccaggtttggctgttcaatattcgtacactttcaatttgggttctaccgccgaaaaagctcagttcgtcaaggactggcaatcctttatctccgcaaagaacttgacgcgtcaattctataataacatggttatctttgacggagacattatccttgagggtttgtttttcggttcaaaggaacaatacgatgccctaggtttagaagatcacttcgctccaaagaaccccggcaacatcttggttcttactgactggttaggtatggtaggtcacgctttggaagatactattttgaaactggttggtaacacaccaacatggttctacgctaagtctttgggttttagacaagataccttgattccttcggctggcatagacgagttcttcgaatatatcgctaaccataccgcaggtactcctgcctggtttgtgacccttagtttggaaggaggtgctattaacgacgtcgctgaagatgctactgcttacgcacacagagatgttctattctgggttcaattatttatggttaatccagtcggtccaatctctgacactacctatgaatttactgatggcttgtacgatgtgctagctagagctgttccagaatccgtcggtcatgcttacttgggttgtccagatcccaggatggaagacgctcaacaaaagtactggagaacaaatttaccaagattgcaagaattaaaagaagagcttgacccaaaaaacactttccatcaccctcagggagttatgccagcc

SEQ ID NO: 69 - DNA, 1476 bp, Artificial Sequence
  Synthetic
atgagatctcagttactacacggacttattggtctggttgccttggtgtcaccttccttcgcagtccccacgaaacgtgaagctgtaacctcttgcttgacaaatgctaaggtcccaatagacgctaagggttcgcaaacttggacccaagatggtacagcctataacttgaggttacaatttgagccaatcgctattgccgttccaactactgttgctcaaatcagcgcagctgtcgcctgtggttctaagcatggcgtttccgtcagtggtaaatctggtggtcactcctacacttctttgggtttgggcggtgaagatggtcatcttgttattgaattggacagactgtactcagtcaagttggctaaggatggaaccgctaagatccaaccaggtgctagattaggtcacgttgctactgagttgtataaccagggtaaaagagcacttagtcatggtacctgtactggtgtaggtttgggtggtcacgctctacacggcggatacggtatggtttccagaaagcatggtttaaccttggactctataattggtgctactgtcgtcttgtacgacggaaaagttgttcactgtagtaagacagaacgttccgatttattctgggccattagaggtgcaggcgcttcttttggtatcgtggctgaattagaatttaacaccttcccagcccctgaacaaatgacctacttcgatattggtttgaattgggaccaaaacactgccgctcaaggtttgtgggaatttcaagaatttggtaaaaccatgccttcagaaatcacgatgcaaattgctatacgtaaggatggatattctatcgatggtgcttacatcggtgacgaagccggtttaagaaaggcacttcaaccattgttgagcaagttaaatgttcaagtctcggcttcgactgtgagctggatgggtctggttacacatttcgccggtactgctgagattaacccaacttctgcttcctatgatgcacacgacactttctacgctacttctttgacaaccagagaattgtcattagaacaattcaagtcattcgtaaactccatcagtaccaccggtaagtcaagttctcattcttggtgggtccagatggacattcagggtggcaaatactctgccgttgctaagccaaaaccaacggatatggcttatgttcatagagatgctttgcttttgtttcaattctacgattcagtgccccaaggtcaaacctacccatctgacggtttctccttactaactactctgagacaatccatttctaaatctcttagagccggcacatggggtatgtatgcaaattacccagactcccaattgaaggctgaccgtgctgctgaaatgtactggggtagcaacctgcctagactacagaagattaaggctgcctatgatcccaagaatatctttagaaatccacaaagtgttaagcctaaggcc

SEQ ID NO: 70 - DNA, 1425 bp, Artificial Sequence
  Synthetic
atgggaaacaccacatcaacttctgctggtcaatgtctattgtccgccgtgggtggcaatccagcattggtcgcttttcagaacgctcctttataccaagccgttgatgtaagaccctataatctggacgttccagttactccagtcgctgttaccacgccagaaactgtcgatcaagttgctagtatagtcaaatgcgctgccgacgctggttacaaggttcaacctaagtctggtggtcactcctacggtaactatggtttgggaggtgtagacggtgaggttgtcgtcgatttaaaaaatttccaacaattctctatgaacaacgaaacctggagggctactattggtgcaggtacattgcttggtgacgtgaccactcgtttgtacaacgccggtggcagagctatggcacatggtacctgtccacaagttggcatcggaggtcacgccactattggtggtttaggtccaacgtcgagattgtggggtgctgccctagatcatatcgaagaagtgcaggttgttcttgctaatagctctattgttagagcttcacaaactgagaaccctgacttgttatttgctttgaagggtgctggtgcctccttcggtatcataacagaatttactgtccgtaccgaaccagctccaggcgaagcagttcaatattcatacaccttcaactttggtgataatgcttccaaggctaagactttcaaagattggcaagccttcgtgtctacaccaaatttgaacagaaagttcgccgctaccatgactgtactggaagacgcaattgttgcttctggtaccttctttggaactaaggaagaatttgatgctttcgaattggagtctcactttcctgaaaatcaaggttccaacgtcacggtcgttcaggattggctgggtttagtcgctgactgggcagaagatgcagctttggaaggaggtggtggtgtcccatccgctttctatgccaaaagtttgaatttcagtccagatactcttatccccaacgacacgattgatgacatgttcgactacttttctaccacagaaaaggatgctttgttgtggttcgccatttttgacctttcgggtggtgctgtgtctgatgtccccgttcactcaacttcttacactcatagagatactctgttttggttacaatcgtacgcaatatctgttggcccagtaagcaacactactatccaattcttggacggtttgtctaatttgctaacctcttcacaacccgaagttcactttggtgcttatccaggttacgttgacccaaaattgccagacggacaattagcttattggggttccaacttgccaaagctagagcaaatcaaggccgaagtagatcctaacgacgtgttccataacccacaatccgttaaaccagctaagcaa

SEQ ID NO: 71 - DNA, 2112 bp, Artificial Sequence
  Synthetic
atggctcagccaccttcctcagcattcgccacctgtctaaatgatgtctgcggaggtcgtagtggctgtgtgggttacccatcggacattttgtatcaaatcaactgggtagataggtacaacttagacataaacttggagccagctgctgttacaaaaccagaaattacggaagatgtcgccgcttttatcaagtgtgctagcgaaaataacgtcaaggtacaagccagatctggtggtcattcttacgctaatcacggtctgggtggcgaagacggtgcattggttatcgatttagagaacttccaacacttttccatgaattgggacaactggcaagctactattggagccggccataagcttcacgacgttactgaaaaactacatgataacggtggtagagctatctcacacggtacctgtcctggtgttggattgggtggtcatgctactattggtggtttgggtccctcttctcgtatgtggggttcctgtttagatcacgtcgttgaagtcgaagttgttactgctgacggtaagattcaaagagcctctgaagatgaaaattcggacttgttcttcgcactgaagggtgctggtgcttcatttggtataatcaccgaatttgtgatgagaacaaacgaagagccaggcgatgttgtcgaatatacgttctctttgaccttctccagacacagagacttgtccccagtttttgaagcttggcaaaacttgataagtgatccagatttagacagaagattcggttccgagttcgttatgcatgaactaggtgctattatcactggtacctttttcggaactgaagaagaatttgaagcaactggtattcctgatcgtattccaaccggtaaaaagtctatcgttgtcaacgactggttgggttctgtcgctcaacaggcccaagatgccgctctttggctgagcgacttaagcaccgccttcactgctaaatctttggctttcaccaaggatcaattgttatcgtctgaaagtattatggaccttatggattacatcgatgacgctaacagaggtacattgatctggtttttgatcttcgatgtgactggaggtagaattaatgatgtacccatgaacgccaccgcctataggcacagagacaaggttatgttctgccaaggttacggcataggtatcccaactttgaacggtaggacaagagagtttattgagggtataaattccttgatcagaagttctgtgcctaccaatttgtccacttacgctggttacgtcgatgcatctttagaatctccacaggactcctattggggtccaaacctagacgctttgggacaagttaaagaagactgggacccatccgatctgttttcaaatccacaatctgttagacccggtcaaaagtccgtagttgattatttcgataacagagcttcgtctaatggttcagaagacagctctggtggcagtaatggaggtacccgtgatgaacaaggtggttgttggtcttggagaagatccggtccagcatttgctgtctttgttgctttattcgtaggtttccctactccacaaacttcttgggtccaaaagcagaacttgcgtgacccagctttagatctgacagacgccgaatcaccttccagaacacctgttgttaacccaaacacgttaacaactgacaccatggccaagttgtctcgtggcgctccaggtggtaaattaaagatgactttgggtttgcccgtcggtgccgttatgaactgcgctgacaattcgggtgcaagaaacctttacattatttcggtcaaaggaatcggtgctagattgaacagactaccagctggtggtgttggtgatatggttatggctactgttaagaagggtaaaccagagttgagaaagaaggttcatccagccgtcatagtcagacaaagtaagccatggaaacgttttgatggtgttttcttgtacttcgaagacaatgccggtgttattgtgaacccaaaaggagaaatgaagggaagcgctatcactggtcctgttggtaaggaagctgccgaattgtggccaagaattgcttctaattcaggtgtcgtcatg

SEQ ID NO: 72 - DNA, 1458 bp, Artificial Sequence
  Synthetic
atgggaaattcggccagcgtggcaggtagagcttgttttgtcgctgctgtaggtcatgatcccaacttggttacattcaggggtgacttactatatgagttccgtattcagccatcatacaaccttgccataccagttcaccctacggtcgtcacctacccaaaaactaccgctcaagttgctgaaatcgtttcttgcgccgctgcacaaaattataagatgcaagcctacagtggcggtcactcttacggtaactacggtttgggtggagaagatggtcatgttgttgtcgacttgaagaacttccaagactttactatggatccagatactcacgttgctaccattggcgctggtacttccttaggtgatctgcaagacagattgtggcacgctggtggtagagcaatggcccatggtagttgtcctcaagtgggtgtcggtggtcacttcaccatcggtggcttgggcatgatgtccagacagtggggtatgtctctggaccatgtcgttgaagctcaagtagtcttggccaattcttctgtggttacggcttccgatactcaaaaccaagatattttttgggccatcaagggtgctgctgcttcgtttggtattgttacaaaattcaaggtaagaacacacggtgttccaaaggccgctatccaatatcagtacaccttctctcaaggtgacgtattagacaaagttaagttgtttatggcttggcaaaacattgtcgctaagccaaatttgactcgtaacttcagtactgaattgaccatattccaagatggaatcatgattatgggtagctttttcggtactagagatgaatttcataagttcgagttagaaaatgatttaccccttcaaggccttggtaatgttgcatatatcaccaactggctatccttggttgctcataccgctgaagactacttgttgagactgacaggtaacgtcttgacttctttttacgccaaatctctatcattcacggctgacgaattgttcaacgagcaaggtcttgttactttgttcacttatttagacgcagctccaaaaggcacacctacctggtgggttatcttcgatttggaaggaggtgccactaacgatgtcccagttaacgctacttcttacgcccacagagatgctataatgtggatgcaaagttacgccgtcgctggttttgaaccaccaggttttattattaagagattcctaaacagattgcatggtgttgtaatcggtaatcgtgcacctggtgctgtccgttcctatcctggttatgtcgacccatacttaagaaatgcccaggaaacctactggggtccaaacttggctagattacaagatattaagacagctgttgatccagatgacgtttttcacaatccacaatccgttaaggtgaatagtctttcgccaccagaccctggaagccatgatgtc

SEQ ID NO: 73 - DNA, 1419 bp, Artificial Sequence
  Synthetic
atgggtcaaacgccaagctctcctctagccgactgtttaaatgcagtttgcaacggaagagataactgtgtggcttttccatccgctccactgtatcagatctcttgggtcgacaggtacaatttggatatagaagtagagcccattgctgttaccagaccagaaactgccgaagacgtttcaggtttcgtcaaatgtgctgccgctcacaacattaaggttcaagcaaagtccggcggtcattcttacgctaactatggtcttggtggtgaagatggtgaattggtcgttgatttgagaaatttccaagattttagtatcgatacaaacacttggcaagccaccttcggcgctggtcacaagttagacgacgtcactgaaaaattgcataagaacggtaagcgtgctatttcacacggtacttgccctggtgtcggtatcggtggtcacgctaccattggcggattaggtcctgagtctcgtatgtggggttcgtgtttggatcatgtgatcggtgtagaagtcgttactgctgacggaagcatagttcatgcctcggacaccgaaaattccgatttgttctttgctcttaaaggcgcaggagcttctttcggtattgtaacatcttttgttgttaagactagaccagaaccaggttccgttgtccaatacagctactctgtcacgttcgcaaaacacgctgacctatccccagttttcagacaatggcaggaattggtaatggatccaggtttggacagaagatttggtaccgaatttaccatgcacgagctgggtgtcattatctctggtactttctatggtactgacgaagagttccaagccacaggtattcctgatagaatcccaaagggtaagatttctgttgttttcgatgattggatggctgttatagcaaaacacgccgaagaagctgctttgtcgttaagtagtatctcctctgcttttaccgcccgttccttggctttcagaagagaagacaagatctcaccagaaactatcaccaacctgatgaactacattgattctgctgatagaggtactttggtctggttcctaatctttgatgctaccggtggtgccatttccgatgtcccaacaaacgccacagcttactcacatagagacaaggttatgtactgtcaaggctacggcgtaggtatacccactttaaatcaacagaccaaggacttcttgtcgggtattattaacactatacaatctggtgccggtaatactttgactacttatcctggttatgtcgatccagctttgaccaacccacaagaatcctactggggaccaaacatcgacactttaagagctatcaagagtcagtgggatccaaacgatatctttcataatccacaatctgttaggccagctgccgtggctgcc

SEQ ID NO: 74 - DNA, 1518 bp, Artificial Sequence
  Synthetic
atgcttaaaaccatcgctgccgttgtattcatttgctcgcaggcttttttggtccgtgcagacctaaagtccgagctgactgctttgggcgtgggtgccgtcttccctggagattcagtttacacgagcgatgctaagccatataacttgagatttgacttcaaaccagctgctataacttttcccaataccccagccgatgtctctcaaattgttcaaatcgccggtaagtacgcacacaaggttgcaccaagaggtggtggtcattcctacatttctaacggtgttggtggaatggacaatagtatcattgctgatatgtctcacttcaagtctattgtagtccatacaaacaatgacactgctaccatcgaaactggtaacagattaggcgatatagctttagctttgttccaatatggtaggggtatgcctcacggtgcttgtccatacgtaggtattggtggccacgccaactttggtggtttcggtttcatctcaagatcctggggtttgaccctagatgttgtcgaagctattgacctggttttagcaaacggcactatcacgacagtctctgctactcaaaacccagacttgtattgggccatgagaggtagcggtagttcttttggaatcaccaccgctatccatgttagaaccttctccgcaccagcttctggtattatcgctttggacacttggtacttgaatcttgaacaagctgttagagccttgagttcctttcaagatttcgctcacaatactgtgactttaccatcttattttggtggtgaatttgtcgttaacgccggtccttccccaggtttgttgtctattacattcttctcgggattttggggtcctccaaatcagtacaactctacgctagcaccatggaaaaattccatgccattccccccaaacacaacttcatactcgcaaggtaactacatagaaagcttgtccgcccgtttcggaggtgctcccttggatacctctctaggtccagataatactgacactttttacgtcaagtcattaatagtcccacaagttaccatttctgatgaaggtgctcaagtaggtattagcgataaagcttggagagctctgttccaatatttgataaacgagcagcctaacctgcctgttgattggttcatcgaagttgaattatggggtggtcaaaatagtgccattaacgccgtcccacaagcttctacagcttttgcttatagagacttgttgtggactttgcaaatgtactcttacaccccaaaccatcaaccaccttacccagacgccggttttgcattcaatgacggcatggctaatagtatcattcataacatgccaaacggttggaattatggtgcttacactaattacgttgataaccgtttagacgattggcagagattgtactatgctaaccactaccccgctttgcaagccttgaagtctaggtatgaccctagtgatacattttcgttcccaacttccattgaactttta

SEQ ID NO: 75 - DNA, 2967 bp, Artificial Sequence
  Synthetic
atgactaccaacggtatacaacccggccatgtcggtaatttaacacaggaccaagaggctaaacttcaacaattgtggtcgattgtactaacgttgttagatgttaagtccttgcaaggtggagatacttctgcccagacccaaccagaccaacgtccaagtactagcttgtctagggctgacaccgttgtgtcagcacacggtcaaactgcttttaccgaagatctatcccaagttttgagagaaaacggtatgtctaatccagatatcaagtccgtcagagaatctctgtccaacacttctatcgacgaattgagatccggtttattgtacacagccaaacacgattcacctgatgtcttgcttctaagattcttaagagctcgtaagtgggacgttggtaaggctttcggtatgatgttgagagcattggtatggagaaaagatcaacatgttgacgacaaggttattgctaatccagagctggccgctttggtcacttctcagaacaccgtcgatacacacgccgctaaggaatgtaaggattttctggaccaaatgagaatgggtaaatgctatatgcatggtaccgatagggacggaagacctgttttagttgttagagtcagattccaccaaccatctaagcaaagtgaagccgtgattaaccgttttatcttgcacacgatcgaaacagctagattgctattggctccaccacaagaaactgtcactattattttcgacatatggaccggtttcggtttgtctaatatggaatacgcccctgttaaatttattatagaatgtttccaagaaaactatccagaatcgttaggctacatgcttattcataatgctccctgggttttttccggtatctggaagatcatcaagggttggatggatccagtcatagtgtctaaagtgaacttcactaacaaggtttcggatttagaaaaattcatcgctccagagcaaattgtaaaggaactaaagggtaaggaggactggacctacgaatatgtcgaacccgtagcaggcgagaacgaattgatggctgacactgaaaccagagataggatttacgcagaaagattgaagatcggtgaagagttgttgttgagaaccagcgaatgggtttccacttcacagcgtaaggacgctgctgccacgactacagctagagaacagcgttctgaaaccatagaaagtttgagacaaaattattggcaactagacccttacgttagaggtagaacttttttggatagaactggtgttgtgaagcctggaggtaagattgacttctacccatctccagatttggagccaagtactgccaaaatgttagaagtcgaacactttgaaagaacccaatttgatccataccttttcttattgccacacggtgctagaattgctgttaggcattgtagcgtcaccgctttaccaacctatcttaaggctcacccacgtggtatgctatctacaatggccttcagtttcctacgtgtattgtcttccctattgctggtcttgcaattatcaaccgctgctagtacttcgacgttgagacaatgtcttttgactgctgttcaaaacgacccaaccctggttgccgttgatggagatttgcttttccaaaccttggctgttcaagtctacaacttgaactggccagtcactcctgctgctgtagcctttcccaaatccgcccagcaagtttcttctatcgttaattgcgcagcatcccttggttataaagttcaagctaagtcgggtggtcattcttacggtaactatggcttaggtggtacaaacggcgcaatctctataaaccttaaaaatatgaagtcattctcaatgaattacactaactaccaagctacggttggtgctggtatgttgaatggagagttagacgattatctgcacaatgccggtggtagagcaattgctcatggcacaagcccacaaattggtgtcggtggtcacgcaactatcggtggtttgggtcctgctgccagacagtacggtatggaattagatcacgtcttggaagctgaagttgtgttagcaaatggtacagtcgtcagagcttcctctacccaaaactcggacttgttgtttgccatcaagggagctggtgcttctttcggtgtggtgactgaatttgtttttagaacagagccagaacctggatctgctgttcagtactccttcacttttggtttaggctccacctcttcacgtgccgacctattcaagaagtggcaatcattcatttctcaaccagacttgactagaaaattcgccagcatctgtaccatcttggaccatgttttggtcatttccggtactttctttggtactaaagctgaatacgacgctttaggtttagaagatcaatttccaggtcacaccaattctactgtgatcgtatttaccgattggttgggactggttgctcaatgggctgaacaatctattttggatttgaccggtggtattccagccgatttctactccagatgtttatcttttactgaaaagactccaattccatcgactggtgtcgatcaattgttcgagtatctggacagtgcagatacgggagctctattgtggtttgttattttcgatttggagggtggtgccattaacgatgtcccaatggatgctacaggttacgctcatagagacaccttgttttggttacagtcttatgccataactttaggttctgtttcccaaactacctacgacttcctggatcgtgttaacgaaataattagaaataacacaccaggtttgggaaacggtgtttacccaggttacgtcgaccctagacttcagaatgcaagagaagcttattggggttccaatttgccaagacttatgcaaattaaaagcctttatgacccatcggacctgttccacaacccccaaggtgttttgcctgct

SEQ ID NO: 76 - DNA, 1437 bp, Artificial Sequence
  Synthetic
atgggccagggtcaatcctctgccggtggtttgcaagactgcttaacgtcagcagtgggtagcggaaatctagctgtaccttctaaacccttctaccaacaaactgatgtcaagccatataacttggatatccacgtccatccagttgctgttacatacccacaaactaacgaggacgttgctgctattgtcagatgtgctaaggaacacgaagccaaagtccagccacgttccggtggtcattcgtacggtaattttgccaccggtaacggaaacgataacatgatagttgttgacttgaagcacttcaagcaattctctatggatgacaatacctggatcgcaactttaggttccggccaccttctgggtgatgtcacaaagaaattgttagctaacggtggtagggctatggctcatggtacttgtcctcaagttggtattggcggtcacgctaccattggtggtctaggtccaatgtctaggatgtggggcagttccttggaccacgttcaagaaatcactgtggtcttggccaattctagcattatcacggcctctccaacccaaaataaggatgttttttgggctatgaagggtgcaggagcctcattcggtataattactgaatttaaagttattacccatccagctccaggtgaggctgttaagtatagtttcggtttttcgggaggttcacacagagatcaagctaagagattcaaaaagtggcaatctatgatcgctgaccctggattgagtagaaaacttgcttctcaagtagttctgagtgaaatcggtatgattatatcaggtacctttttcggtacccaggctgaatacaaccaattgaacttaacttctgtcttccctgaaatgtcctcccataagattatcgtatttaacgattgggctggtctagtgggtcactgggccgaagacgtgggtttacaattgggtggtggaatctcttctccattctactccaagagcttggctttcaccccaaacgacttgattcctgctgaaggtattgacagatttttcgaatatttggatgaagttgataagggtactttgatctggtttggtatattcgatttggaaggtggcgccactaacgatattccagcagacgcaactgcatacggtcatagagatgcattgttttatttccagtcatatggtgtcaatctaggattaaaggttaaggatgagacaagagactttatcaatggtatgaatagcgtccttgaaggttctttgagcaaccacaaactgggtgcttacgctggttacgttgatcccgctctttctttggaagccgcccaggttggttactggggtgacaacttaccacgtctgagacaaattaagagagctgtagatccagacgacgttttccataatttgcaatccgtcagaccagctgcttcc

SEQ ID NO: 77 - DNA, 1416 bp, Artificial Sequence
  Synthetic
atgggtaataagccatccactcctttagcccattgcttgagagatgtttgtgcaggaaggggtaactgtgtcgctttcccaaacgagtatctttaccaggctaactgggtaaaaccctacaatttggacgtgccagttaagccaattgctgtctttagacctgataatgccgctgacgtcgctgctgctgttaagtgtgccggtcaatcatcggttcacgttcaagcaaaatctggtggccactcttatgcaaacttcggtctaggtggtggtgatggtggtttgatgatcgacctgcaacatttgaacaagtttagcatgaacaacgaaacctggcaagctacattcggatccggtttcctattgggcgatttagacaagcaactgcacgctaatggtaatcgtgccatggctcatggtacttgcccaggtgttggcataggtggtcacgccaccatcggaggtattggtccatcttccagaatgtggggtacggctttagatcacgtattggaagtcgaagttgtgactgctgatggtaaaattcaaagagccagtaagacccagaactctgacttgttttggggtttgcaaggtgctggtgcttcattcggcatcataactgaatttgttgtccgtaccgaacctgaaccaggttctgtcgttgagtacgcctactctctaaatttcggcaaacaagcagatatggctccagtgtataagaagtggcaagaccttgtgggtgaccctaacttagatagaagattcaccagtttgtttattgccgaaccattgggtgttttgatcactggtacattctacggtaccctagacgaatacaaggcttccggaatcccagacaagttgcccgcttcgggtgcctccattacagtcatggattggttgggtagcttagctcacatcgctgaaaaaactggtttatatttgtctaacgtatctactaaatttgtttccagatcattagcattaagggaagaggaccttttgagcgaacagtccattgatgatttgtttaagtacatgggctctgctgacgctgacacaccattgtggttcgttattttcgataacgaaggtggtgccatcgctgatgtccctgataattctactgcttatccacatagagacaagattatactgtaccaaagttactccgttggtttgttgggagtttctgacaagatgataaatttcgtcgatggtattcaagatcttgtacaaaagggcgctcctaacgcccacacgacttacgctggttatatcaacgctaacttagacagaaatgctgcccaaaaattttattggggtgacaagttgccacagctgcaacaactaaagaagaagttcgacccaacatcgttattcagcaatccacaatctattgatccagccgat

SEQ ID NO: 78 - DNA, 1422 bp, Artificial Sequence
  Synthetic
atgggtaacaccacttccatcgcagccggcagagattgtttggtttcagctgtcggtccagctcatgtgacatttcaagacgctctgctttatcagacgaccgccgttgatccttacaatttgaacattcccgtaactccagctgctgtcacatacccacaatcggccgaagagatagctgctgttgtcaaatgcgcttccgactatgattacaaggttcaagcacgtagtggaggtcacagcttcggtaattacggtctaggtggtcaaaacggtgccatcgtcgttgacatgaagcacttctctcaattttctatggatgaatctactttcgttgctaccattggtccaggtactacgttaggcgacttggataccgaactatataatgctggtggtagggccatggcccatggtatctgtcctactattagaactggcggtcacttaaccgtcggtggattgggtccaacagccagacagtggggtctggctttggatcatattgaagaggtagaagttgttttggctaactcttccatcgtgagagcatcgaacactcaaaatcaagacattttattcgctgtaaagggtgcagctgcttcttttggtatagtcaccgaatttaaagttagaactcaagaagctccaggtttggctgttcaattctccttcaccttcaacttgggttctcctgcacaaaaggctaagctagtcaaagattggcaagcatttattgctcaggaaaacttgagctggaagttctactcaaacttggtcatcttcgacggtcaaataatcttagaaggtattttctttggatcgaaagaggaatacgacgaactagatttggaaaagagatttccaacgtcagagcccggcactgttttggttttaacagattggctgggcatgatcggacacgctttggaagatactattttgaagttggtgggtgacaccccaacgtggttttatgctaagtccctgggtttcactccagacactcttatcccagattctgccattgatgacttcttcgactacatccacaagactaacgctggtaccttagcttggttcgtaaccttgtcattggaaggtggtgcaattaattctgtttcggaagatgctacagcttatggtcatagagatgtcttgttttggtttcaagttttcgttgttaatcctttaggtcctattagtcaaaccacgtacgatttcactaacggcctgtatgacgtccttgcccaagccgtaccagaatccgccggtcacgcttacctaggttgtccagaccctaaaatgccagacgctcaacgtgcctactggcgtagtaacttgccaagacttgaagatttgaagggtgacttggacccaaaggatactttccataatccacagggtgttcaagttggtcca

SEQ ID NO: 79 - DNA, 1488 bp, Artificial Sequence
  Synthetic
atgctgtcaaccatggcattcagctttgtccttagaattttatctccattgttcttgatcctacaattatctactgccgctagtacatccactttgaggcagtgtttgttaaccgctgttcaaaatgaccctacgttggtagctgttgatggtgatttgctgtaccaaactcttgccgtgcaagtctataacttgaactggccagttacccccgctgctgtcgcctttccaaagtcgactcaacaagttgcttctatagttaactgcgctgcatccttgggatacaaagtgcaagctaagtctggcggtcattcctacggtaattatggtttgggtggtaccaatggtgccatttcaatcaacttaaagaacatgaaatcgttctctatgaactacacgaattaccaagccacagttggtgctggtatgcttaacggcgagttagacgaatatttgcacaacgctggtggtcgtgctgtcgcacacggaacttcccctcagattggtgtaggtggtcatgctactattggaggactaggtccatcggctagacaatacggtatggaattggatcacgtcttagaagccgaagttgttttggcaaacggtaccgtagtccgtgcttcttctactcagaatagcgacttgctgttcgccatcaagggtgctggtgctagttttggtgtcgttacagagtttgtgttcagaacagaaccagaaccaggttctgctgttcaatataccttcactttcggcttgggttccacctctgccagagccgatctatttaagaaatggcaatccttcatatcccaaccagacctgactagaaagtttgcaagtatctgtaccttgttagatcatgttttggtcatttctggtactttctttggtacaaaagaagaatacgacgctttgggcttggaagatcaatttcccggacacactaactctactgttatcgttttcaccgattggttgggtttggtggctcaatgggctgaacaatcaattttagacctgactggtggtatcccagctgatttctacgcaagatgtttgagctttactgaaaagaccctaattccttccaatggtgtcgaccaattattcgagtacctagactcagcagatactggtgctttgttatggttcgtcatctttgatcttgaaggtggtgccattaacgacgtcccaatggacgctaccggctatgctcacagagataccttgttttggctacagtcttacgctattacgcttggttctgttagtgagactacctacgatttcttggacaatgtaaacgaaatcataagaaacaatacaccaggacttggtaacggtgtttaccctggttatgttgatccaaggttgcaaaatgcaagagaagcctattggggttcaaatcttccacgtttgatgcaaattaagtctctatatgacccaaccgacttgtttcataacccacaaggtgttttgcctgcc

SEQ ID NO: 80 - DNA, 1404 bp, Artificial Sequence
  Synthetic
atggctccatccatttcattttctttgctacaaatctcgcttttggcctattctggtctggtgagtggagatttctctttaagacagtgcttggaatccgctgttagcagggtagcattcgagggcgaccctttttaccaattattgtcagtcagaccatacaacttagatatatccattgttccagctgccgtcgctttccccgctgacactaatgaagttgcagctgtcgtaagatgtgctgcccaaaacggttatcaagttcaagcaaaaagtggtggtcactcatacgctaatcatggtttgggtggtaccaacggagctgttgtggttaatctggaaaacttgcaacacttctccatgaacacgactacctgggaagccacaatcggtgctggtacattattgggtgatgtcaccaagcgtttgtctgacgctggcggtagagcaatggcccatggtacttgtcctcaggttggttctggaggtcactttactattggtggcctaggtccatctagtagacaatttggcgccgctttggatcatatcatagaagctgaagtcgttctagctaactcttctattatcagagcatctgagactgaaaacccagatgtgttcttcgctgtaagaggagctgcttccggttttggtattgttaccgaatttaaggttcgtaccgaaccagaaccaggtcaagccgtcagatacagttattctttctcgttcagcgacaccgctacgcgtgcagacttgttcaagaaatggcaagcctacgtcactcaaccagatttgcctagagaacttgcttctactctgacaattttggaacacggtatgttcatcactggtacgtttttcggttcaaaggaggagtacaatgctctaaagattgaaaccgaatttcccggtttcgccaagggtggaaccttagtcttggatgactggttgggtttagttagtaattgggctgaagacttgcttttgtcggaagaagaaatcgagcaaatgttcgaatatattgataacgttgacaaaggtacactactgtggtttgccattttcgacctacaaggtggtgctgtcggtgatgtaccagtcgatgccactgcttacgctcacagagataccttgatatggctacaatcctacgcaatcaatctgtttggtagaataagcgaaactactgttgagtttttagaacgtttgaacgaattgactttgacatctacagctaagacggttccatatgcagcctaccctggttatgttgacccaagattgactgatgctcaagctgcctactggggatcgaacttagctagattgaacagaatcaaagctgaaatcgacccaaacaatgtattccacaatccccaatccgttcgtccagcttctggt

SEQ ID NO: 81 - DNA, 1425 bp, Artificial Sequence
  Synthetic
atgggtaatactacctcgatagccgctggcagagattgcctggttaacgctgtcggtggtaaccaggcattagtagcttttcaagaccaattgctatatcaatccacggccgtcgaagcttacaacttgaatattcctgttacaccagctgctgtcactttcccagagtcttcagaacaaatcgcagccgtggttaaatgtgcttctgaacacgactacaaggttcaagctcgtagcggtggacatagtttcggtaattatggtttgggtggtaccaacggcgccatcgtggttgatatgaagaaatttgatcaattctccatggacgaatcgtcttacattgctactattggtcccggtaccactttaggtgatgtcgacacagaattgtacaacgctggaggtagagccatggctcacggtatttgtccaaccatcagaactggcggtcatcttacgatgggtggtttgggtccaactgccaggcagtggggcttggctctggaccacatagaagaggttgaagtcgtattagctaattcttccatcgttagagcatctcatacccaaaaccaagatattttgtttgccgttaagggtgcttccgcatcattcggtattgtcactgaatttaaggttagaactgaacctgcaccaggtttggctgtccaatactcttataccttcaatttgggtagtgcagcctccaaggctaaattagttaaggattggcaagagttcatcgctcaggacaacttgacatggaaattctatagcaatatggtcattatcgacggagatataattctggaaggtatctttttcggttctaaggaagaatttgatgctttagaactagaaaacaggttcccacccaagaacccaggtaacatacttgtgttgactgattggttgggaatgatttctcactccttggaagacatcattttaagagttgctggtggtgtaccaacctacttttacgctaagtccttaggtttcacacctcaagctttgatcccatctagcgctattgatgacctgttcgattatatagaaaagactaatccaggtactctagcctggtttatcaccttgtccttggagggcggagctattaacaacgttccagctgacgcaacagcctacggtcacagagatgtgcttcattgggtccaaatctttgccgctaatcctttgggtccaatttctgaaaccacttacgacttcactgacggtttatacaacatccttgctaaagccgttcctgagtctgctgaacatgcttatttaggttgtcctgatccacgtatgaaagacgctcaaaaggcttactggagagataacctgccacgtttggaagaattaaaggctgaattggatcccaaagatacttttcacaatccacaaggtgtagccgtcgct

SEQ ID NO: 82 - DNA, 1464 bp, Artificial Sequence
  Synthetic
atgttattgaaactatttttcttggccgtagcagcttcagttgctctggctgcttccagtgaggccttgaagcagtgcttggaaaacgtcttcactgaccgtgcaggctttgctttcgccggtgatttattctatgacagaatagttaatagatacaacttgaatatcccagtcaccccttcggctttggcttttccaacgagctctcaacaagttgccgatattgtgaagtgtgcagctgataacggttaccccgttcaagctaggtccggaggtcattcttatggtaactacggtcttggtggtgctgacggcgccgtcgctatcgatttaaaacacctacaacaattctctatggacaagacaacttggcaggctaccattggtgccggatctttgctatccgatgttacccaaagattgagccacgctggtggcagagccatgtctcatggtatttgtccacaagtcggttcgggtggtcacttcacaatcggtggtttgggaccaacttcaagacaatttggtgctgccttagaccatgttcttgaagtcgaagtcgttttggctaattccagtattgtccgtgcttctgatactgaaaacaaggatttgttttgggctattaagggtgctgcatctggatacggtatcgttaccgaatttaaagtgagaactgaacctgaaccaggtaccgctgttcaatatgcatacagcatggagttcggtaatccaactaagcaagcaacccttttcaagtcctggcaggcttttgtgtctgacccaaaattgactagaaagatggcctctacattaacgatgctggaaaacagtatggctatatccggtactttcttcggtactaaggaagaatacgacaagttgaatttgaccaacaagtttcctggtgctaatggtgacgctttagttttcgaagattggctgggcctagtggctcactgggctgaggatttgatattgggtttagctgccggtattccaactaacttctatgccaaatcaacgtcttggactccccaaacattaatcacccccgaaaccgtagataaaatgtttgactacatcgccaccgttaacaaaggtactcttggctggttcttattgtttgacttgcaaggtggttatacgaacgatattccaaccaacgccacatcatacgctcacagagatgtcttgatttggctacaatcttatacagttaactttttgggtcctatctcccaggctcaaattgacttcctagatggtttgaataagattgtcaccaacaataagttgccatacactgcttacccaggttacgttgatccattgatgccaaatgctccagaagcatactggggaactaacttgccaagattacaacaaatcaaggaattagtcgaccctaatgatgtttttcgtaacccacaatctccatccccagctaacaaagagccactg

SEQ ID NO: 83 - DNA, 1416 bp, Artificial Sequence
  Synthetic
atgggtaacggaaatagcacaccttttcgtgactgtttagattctatatgcgcaaacagatccacctgtgtgacgtatccaggtgacccactgttctcgtgttggagtaggcccttcaatttggagtttcctgtagtcccagccgctatcattagaccagaaactaccactgaagttgctgaaactgttaaatgtgctaagaagtacggttacaaggttcaggctaaatcaggtggccactcctacggtaaccatggtttgggtggtgtcggaggtgccgtcagtattgatatggtcaacctaagagatttctctatgaacaataagacctggtatgcttctttcggttctggtatgaaccttggtgaattggacgagcacttacatgccaacggcagaagagcaatcgctcacggtacatgcccaggtgttggtactggtggtcatttgaccgttggtggtttgggtccaatttccagacaatggggctctgctctggaccacttgctagaaatcgaagtcatcactgctgatggtacggtgcaaagagcctcatatactaaaaattctggattattttgggctttgcgtggtgctggcgcctctttcggtattgttacaaagtttatggttaagactcacccagaacctggtagagtagtgcaatactcatacaatatagctttggcctcccatgctgaaactgctgaactatatagggaatggcaagccttggttggagatccaaacatggaccgtagattctcttccttattcgtcgtccaaccattgggtgctttgattaccggtaccttctttggtaccaagtcccaataccaggcaactggtattcctgacagactaccaggtgctgataaaggtgctgtctggcttacagattgggcaggcttgttattgcacgaagctgaggccgctggttgtgccttaggtagcatcccaaccgctttctacggcaagtcgttgtctttgagtgaacaagaccttttatcagattctgctattaccgacttgtttaagtatttagaggataacagatccggtttagcccccgttactatcttgtttaataccgaaggtggtgctatgatggatacgcctgccaacgccactgcttacccccacagaaactccattatcatgtaccaatcttatggtataggagttggtaaggttagtgctgcaacacgtaaactgttggacggtgttcatgaaagaatccaaagaagcgcaccaggcgctctgtctacttacgctggttatattgacgcctgggctgaccgtaaggctgcccaaaagctatactgggctgataatttgccaagattaagagaattaaaaaaggtctgggatccagcagatgttttctcaaacccacagtctgttgagccagcagac

SEQ ID NO: 84 - DNA, 1113 bp, Artificial Sequence
  Synthetic
atggattctaacacttgggaggccacgttcggctcaggatttttacttggtgaactagacaaacatttgcacgctaatggtaacagggctatggcacacggtacctgtccaggtgttggtatgggtggtcatgccactatcggaggtattggccctagctccagactgtggggtacaaccttagaccacgtattgcaggtcgaagtggttactgctgatggtaagatacaacgtgcttctaagactcaaaacccagatttgttctgggctctacaaggtgctggtgcctcgtttggcattatcaccgaatttgtcgttagaaccgaacccgaaccaggtagtgttgtcgaatacacctattccgtatctttgggaaagcaatctgacatggctccattgtacaaacaatggcaagctttggttggtgatccttccctggacagaagattcacaagtttattcattgccgagccattgggtgttttaatcactggtacattttatggtactatgtacgaatggcacgcatcaggtatccctgataagttgccaagaggtccaatttcggtcaccgttatggactctttgggatctttagctcatattgccgaaaaaactggcctgtacttgaccaatgtcccaacgtccttcgctagcagatctcttgccttgagacagcaagatttgttgtccgagcaatctatcgatgacttattcgaatatatgggttcggctaacgcagacactccactttggttcgtgatctttgacaacgaaggtggtgctattgctgatgtgcctgataatagcaccgcctacccacatagagataaggttattgtttaccaaagctactccgtcggtttactaggtgtcactgataaaatgataaagttcttggacggtgttcaagatattgtccagaggggagctcccaacgcccacacgacctatgcaggttacatcaatccacaattggaccgtaaggctgctcaacaattctattggggtgacaagctaccaagattgcaacagattaagaagcaatatgatcctaacaacgtgttttgcaatccacaatctatctacccagctgaagacatgtctgacggt

SEQ ID NO: 85 - DNA, 1419 bp, Artificial Sequence
  Synthetic
atgggtaacacaacttccatcgcaggcagagattgcttagtctcagcccttggaggtaattctgctttggctgctttcccaaaccaattgctgtggaccgccgacgttcacgagtataatttgaacctacctgtaacgccagctgccataacctaccccgaaactgctgaacagattgctggtatcgttaagtgtgctagtgattacgactataaagtgcaagctaggtctggtggtcattcctttggtaattacggtttgggaggtactgatggtgccgttgtcgtcgacatgaagcacttcaaccaattctcgatgaacgatcaaacctacgaagcagttattggtccaggtactaccttaaacgacgttgacattgaattgtacaacaatggcaagagagctatggctcatggtgtttgtccaactatcaaaacaggtggtcactttacaattggcggtctgggtcctactgccagacaatggggtttggctttagatcacgtcgaagaagtggaagtagtcttggccaactcttctatcgttcgtgctagcaatacccaaaaccaggatgtcttctttgctgtcaagggcgcagctgccgacttcggtatcgttacggagttcaaggttagaactgagccagcacctggtttagctgttcaatattcgtatacctttaatcttggtagtactgctgaaaaagcccaatttgtcaaggattggcaaagcttcatttccgctaaaaacttgactcgtcaattctacaacaatatggttatatttgacggtgacattattttagaaggtttgtttttcggatcaaaggaacaatacgatgccttgggtttggaagatcattttgctccaaagaatccaggtaacatcctagtgctgacggactggttgggaatggtaggtcatgctttggaagacaccattttgaagctagttggaaacacacccacttggttctacgctaaatctttgggtttcagacaagataccctaatcccatctgctggtattgacgaatttttcgaatatatagcaaaccacaccgctggtactccagcttggttcgttaccttatctctggaaggcggcgctataaacgatgtggctgaagatgccacagcatacgcacacagagatgtcctattttgggttcagttgttcatggtcaatccactaggtccaatctcagaaactacctacgagttcactgacggtttatatgacgtcttagcaagagctgtccctgaatctgttggtcatgcctatttgggttgtccagacccaagaatggaaaacgctccacaaaagtactggcgtactaatttgcctagattacaagaattgaaagaggaattggatccaaagaacaccttccaccatccacaaggtgtgattccagct

SEQ ID NO: 86 - DNA, 1428 bp, Artificial Sequence
  Synthetic
atgggtaacactacgtcgattgccgcaggcagagattgccttgtcagtgctgttggtggtgtggctgctcatgttgcttttcaggactctttgttataccaagccacagccgtagagctgtataatctaaacatacctgtcacccccgctgctgttacttacccacaaagcaccgatgaaatcgccgctgtcgttaaatgtgcttcagactatgactacaaggttcaagctcgttccggtggtcactccttcggaaactacggtttgggtggccaaaatggtgcaattgtaatcgatatgaagcacttctctcaattttctttagataagtctactttcattgccaccttcggtccaggtactacattgggaaacttggacaccgaactatatcatgctggtaacagagcaatggctcacggtatctgtccaactattagaaccggaggtcatttgacaatgggcggtttgggtccagctgccaggcagtggggtttggcattagatcacgttgaagaagtcgaagttgtccttgctaattccagcgtggtaagagcctctgacactcaaaatcaagacgttttctttgctgttaaaggtgctgctgcttcttttggtatcgtcactgagttcaaggttcgtactgaagaagcccctggtttggctgttcaatacagctttccattcaacttgggtaccccagctgaaaaagctaagttagttaaggattggcaagcatttatagctcaagaaaatttatcgtggaagttctactcaaacatggtcatctttgatggtcaaattattctggagggcattttcttcggctccaaaaaggaatatgacgaattggacctggaaaacaagttccccacctcggaaccaggtacagtcttggtcttgaccgattggcttggtatgatcggtcacggtttggaagacactattttaagattggtgggtaactccccaacatggttctacgccaagtctcttggctttactccttctactttaattagtgatagtgctatcgatggtttgttcgattatatccacaaaaccaacccaggtacattggcctggtttgttacgctatctttggagggtggagctataaatactgtctccgaagatgccactgcttacggacatagagatgttttgttctgggttcaaatctttgttgctaaccctttgggtccaatttcacagactacctacgacttcgctgacggattatacaacgttctggctcaagctgtgccagattctgccggtcatgcttacctaggttgtccagaccctaaattgccagatgctcagagagcatactggaggtctaatctaccaagactggaggaacttaagagagacttggatccaaaagacatcttctataatccacaaggtgtccaaattgtttcc

SEQ ID NO: 87 - DNA, 1428 bp, Artificial Sequence
  Synthetic
atgggtaacaccacatcgatcgctgccggacgtgactgcttactttccgcagtcggtggcaatcatgctcacgtggctttccaagatcagctgctataccaagccactgctgttgaaccttataacttgaatataccagtaacgcccgctgccgttacttacccacaatcagctgacgaggttgctgccgtcgttaaatgtgcagctgattacggttataaggtccaagctagaagtggtggtcactcttttggtaactacggtttgggtggcgaagatggtgctattgttgtggacatgaagcatttcgatcaattttctatggacgaatctacctatacagccactattggtccaggtatcactttgggcgatttggacaccgctttatacaatgcaggtcacagagccatggctcacggtatttgtccaaccatcaggacgggtggtcacttgactataggaggtttaggtcctactgctagacagtggggacttgccttggatcatgtagaagaggttgaagtcgttctggctaacagctccattgtcagagcctctgacacacaaaaccaagaaatcttgttcgccgttaagggtgctgctgcttccttcggaatcgtcaccgaatttaaagttcgtactgaagaagctccaggtttggctgtccaatactccttcacctttaatttgggtactgctgccgagaaggcaaagttagttaaagattggcaagccttcattgctcaagaagatcttacttggaagttctattctaacatgaacataattgacggtcaaatcattctggaaggtatctatttcggttcaaaagccgaatacgacgctttaggtttggaagaaaagtttccaacttctgagccaggcaccgtgttggtactaactgactggttgggtatggttggtcatggtttggaagatgtaattttaagattggtcggtaatgctcccacctggttctacgctaagtcgctaggttttgcaccaagagctctaattcctgattccgcaatagacgattttttcgaatacattcacaaaaacaatccaggtaccgtttcatggtttgttaccttgtctttggaaggcggtgccatcaacaaggtccccgaagatgctactgcttatggccatagagatgttctattctgggtccagattttcatgatcaacccattgggtccagtttctcaaacaatttacgatttcgctgacggtctgtatgacgtcttagctaaggcagtgccagaaagcgccggtcacgcatacttgggttgtccagatcctcgtatgcctaacgctcaacaagcctactggagaaacaacttgccaagactggaagagttaaagggtgatcttgacccaaaagacattttccataatccacaaggtgtcatggttgtctcc

SEQ ID NO: 88 - DNA, 1419 bp, Artificial Sequence
  Synthetic
atgggtaacggacagtccacccccttgcaacaatgtttaaatactgtttgcaacggtagactaggttgtgtagctttcccaagtgatgcattgtatcaagccgcttgggtcaagccttacaacctggacgtgccagttacgcctatcgctgtttttaaaccaagctctacagaggatgtcgccggtgctataaagtgtgctgttgcctcgaatgtgcacgttcaagcaaagtccggtggccattcttacgctaacttcggtttgggtggtcaagacggagaattaatgattgacttggctaaccttcaggattttcacatggacaaaacttcttggcaagctactttcggtgctggttataggttaggcgatttggataagaagttgcaagccaatggtaatagagccattgctcatggtacctgtccaggagtcggtatcggtggtcacgccactattggtggtctaggtccaatgtcacgtatgtggggcagtgctttggaccatgtcttatctgttcaagtagtgaccgctgatggttccatcaaaaacgcatccgaatctgaaaactcagatctgttttgggctttgagaggagctggtgccagcttcggtgtcataaccaagttcacagttaaaactcaccctgctcccggttctgtcgtacaatacacttacaagatttcgttgggttctcaggcccaaatggctccagtttatgcagcttggcaagctttagctggtgacccaaagcttgacagacgtttctctacattgtttatcgctgaaccattgggcgccttaatcaccggcaccttttacggaactaaagctgagtacgaagccacgggtattgctgcaagattgccatccggtggtactcttgacctaaagcttttggattggttgggttccttggcccacattgctgaagttgtcggtcttactctaggtgacataccaacctctttctatggtaagtcattggccttgagagaagaagatatgctagatagaacctcaatcgatggtttgttcagatacatgggtgacgctgatgccggtaccttgttatggtttgtcatttttaattcggaaggtggtgcaatggcagatacgccagctggcgcaactgcatatcctcatagagacaaactaatcatgtaccaatcttatgttattggtatcccaactctgacaaaggctaccagggacttcgctgatggtgttcacgacagagttagaatgggtgctccagctgctaacagtacttacgctggatacattgatagaaccttatctcgtgaagccgctcaagaattttactggggtgcacaattgcctaggttgcgtgaggtcaagaaggcttgggacccaaaggatgttttccataatccccaatccgtagacccagctgaa

SEQ ID NO: 89 - DNA, 1419 bp, Artificial Sequence
  Synthetic
atgggcaatactacgtctattgctgccggtagagactgtcttatcagcgcagtcggtgctgctaacgtagcctttcaagatcagctgctataccaagctacagctgtgcaaccctataacttaaatatacctgttactccagctgccgttacctacccacaaagtgccgacgagatcgctgccgttgtcaaatgcgcttcggaatatggttacaaggtccaagctaggtcaggtggacactccttcggtaactacggtttgggtggccaagatggtgcaattgttattgaaatgaagcatttctctcagttttctatggacgaatccaccttcatcgctactattggtccaggaatcaccttgggtgatttggatactgatttatataacgccggtcacagagctatggctcatggtatatgtccaaccatcagaacgggtggtcacctaacagttggtggtttgggccctactgctcgtcaatggggcttagcattggaccatgtagaagaagtcgaagttgttctggctaactcttccattgtccgtgcttctgacactcaaaatcaggatttgtttttcgctatcaagggtgccgccgcttccttcggtattgtaacagaatttaaagttagaaccgagcaagctccaggtatggcagtccaatacagttacaccttccaccttggtacttcagctgaaaaggccaagttcgtcaaagactggcaagccttcattgctcaagaaaacttgacttggaagttttataccaacttggttatattcgatgatcaaatcatcttggagggaatatactttggtactaaagaagaatacgacagcttaggtcttgaacaaagattcccaccaactgacgcaggtactgtgttaattttgacagactggttggcaatgattggtcatggattggaggatacgattttaaagttggttggtgatacacccacctggttttatgccaagtctctaggtttcaccccaagagctcttattccagatagcgctatcgacgaattttttgactacatacacgagaataaccctggtactttggcttggttcgtcacgttatctttggaaggaggtgctatcaacgctgttccagaagatgcaaccgcttatggtcacagagatgtcttattctggttccaattgttcgttattaatcctttgggtccaatctcgcagactacttacggtttcgccgacggtctttacgatgtcctggctcaagcagttcccgaatctgcttcgcatgcatacatgggttgtccagatccaagaatgccaaacgctcaacgtgcttactggagatccaacttgcctaaactggaagaactaaagggctatttggacccagaagacatttttcacaatccacaaggtgttgtaccctct

SEQ ID NO: 90 - DNA, 1428 bp, Artificial Sequence
  Synthetic
atgggtaacaccacatcaataactgctggccgtgattgcctgacttccgccgtcggtggagttgctgcacatgtagcttttcaagacgccttactatatcagaccccagctgtggacccttacaatttgaacattccagttacgcccgccgctgttacttacccacaaagcgctgatgaagtcgccgctgtcgttaagtgtgcttcggattataattacaaagttcaagctagatctggtggtcactccttcggtaacttcggtttgggtggacaaaatggtgcaatcgtcgttgacatgaagcacttttctcaattctctatggatgagagtaccttcgtcgccactattggtccaggcacaacccttggtaacttggacactgaaatctacaacgctggtaagagggctatgtctcatggtatttgtcctagtatcagaaccggtggtcacttgactgtaggcggtttaggtccaacagctagacaatggggtttggctcttgaccacgttgaagaagtcgaagttgtgttggccaactcatccattatcagagcttctgatacccagaaccaagatgtcctatttgcaattaaaggtgctgccgcatccttcggaatagtaaccgaatttaaggttagaactgaagaggctccaggcttagctgttcaatactccttcactttcaatctgggtacgccagctgaaaaggcaaagttggtgaaagactggcaagcctatatcgcacaggaaaatttgacctggaagttttattctaaccttattatctttgacggtcaaattatcttggagggtattttctttggtagcaaggaagaatacgatcaattaaacttagataagaaattccctacttccgaaccaggtacagttttggtattgactgactggttaggcatgattggtcatggtttggaggacaccattctgcgtttagttggtgattctccaacatggttttacgctaagtctttgggtttcacaccttctaccttgatatcaggcagtgctatcgacggtttgttcgattacattcacaaaactaatgcaggaactctagcttggtttgttacgttgagtttagaaggtggtgccataaacgctgtcccaaaggacgctactgcatatggtcatagagatgtcttgttctgggttcaaatcttcgtcgccaacccacttggtccaatttcgcaaaccacttacgatttcaccgatggtctttacgacatcctggctcaggctgttcccgaatctgccggtcacgcttatttgggttgtcccgatccaaagatgccagacgctcaaagagcttattggagatccaatctgcctcgtttggaagaattgaagggtgatctggaccccaaggatattttccataatccacaaggagttcaagtagcatca

SEQ ID NO: 91 - DNA, 1449 bp, Artificial Sequence
  Synthetic
atgaatccttcaattccatcttcctctatgggcaacaccacttccatcgctggtagggattgtctggtcagcgccttaggaggtaacgctggtttggtagcattccagaatcaaccactataccaaacaactgctgtgcacgaatataacttgaacataccagtcacccccgccgctattacgtacccagagactgctgaacaaatcgcagctgttgttaaatgcgccagtcaatatgactacaaggttcaagctagatcgggtggtcattcttttggtaattacggtttgggcggtacagacggtgccgttgtcgttgatatgaagtatttcaaccaattttctatggacgatcagacttacgaagctgtcattggtcctggtaccactttaggtgacgtcgatgtagaattgtacaataacggaaagagagctatggcccacggcgtttgtccaaccatctccactggtggtcatttcacgatgggtggtcttggtccaactgctcgtcaatggggtttggctttggatcacgtggaggaagttgaagttgtcttagcaaattcatctattgttagagcaagcaacacacagaaccaagaagtcttctttgctgtgaaaggcgctgccgcctcgttcggtatcgttactgaatttaaggtaagaacccaacccgctccaggaatagctgttcaatattcttacaccttcaacttaggttcttccgccgaaaaagcccaattcattaaggactggcaatctttcgtatccgctaagaatttgaccagacaattttacacaaatatggttatctttgacggtgatattattttggaaggtcttttcttcggttccaaagaacaatatgaggctctgggtttggaagaaagatttgtccctaagaacccaggcaacatcctggtcctgactgattggctaggtatggttggtcatgcattggaagacaccatactaagattagtcggcaacaccccaacctggttttatgctaagtccttgggttttactcctgacactttgattccaagtagcggtatcgatgaatttttcgaatacatagaaaataacaaggctggtacttccacctggttcgttactttgagtcttgaaggtggtgctattaacgacgtcccagccgatgctactgcttacggacaccgtgatgttctattctgggtacagatcttcatggtttctcctacaggtccagttagttctacgacgtatgattttgctgatggtttgtacaatgtgttgaccaaagctgttccagaatcagaaggtcacgcttatttaggatgtccagacccaaagatggccaacgcccaacaaaagtattggagacaaaacttgccaagattggaggagttaaaggagacattggatcctaaagacactttccataatccccaaggaatcctaccagcc

SEQ ID NO: 92 - DNA, 1419 bp, Artificial Sequence
  Synthetic
atgggtaacacaaccagtatagccggacgtgattgcttgatttcagcacttggtggcaattccgctctagctgttttcccaaacgagttgctgtggacggctgacgtgcacgaatataacttaaatttgcccgtaactccagccgctattacctaccctgaaactgctgcacaaatcgctggtgttgtcaaatgtgcttctgactacgattataaggttcaggccagatctggtggtcattcgtttggtaactacggtttgggaggtgcagatggcgctgtcgttgtggacatgaagcacttcactcaattctcaatggatgacgaaacctacgaagctgttattggtccaggtactacattaaatgacgtcgatatcgaattatataacaacggtaagagagccatggctcatggtgtctgtccaaccatcaaaactggtggtcactttaccatcggtggtttgggtcctactgctaggcaatggggcctagccttggatcatgtcgaagaagttgaagttgttttggctaattcttccattgttagagcttctaacactcaaaatcaagacgtattctttgccgtcaagggtgccgctgctaattttggaattgtaacagagttcaaggtcagaactgaaccagcaccaggtttagctgttcaatacagctacaccttcaacttgggatccaccgcagaaaaagctcagttcgtgaaggactggcaatcttttatctccgctaaaaaccttacgcgtcaattctataacaacatggtcatattcgatggtgatattatattggagggtctgttttttggtagtaaagaacaatacgacgctttgggtttggaagatcacttcgcaccaaagaaccccggcaatatcttggttttaactgactggcttggcatggttggtcacgctttagaagacacaattttgaagttggtcggtaatactccaacctggttctatgccaagtctttaggttttagacaagatactctaattcctagtgccggaatcgatgaatttttcgaatacattgctaatcatactgctggtactccagcatggttcgttacgttgtccttagaaggtggtgctataaacgatgtcgccgaagatgctactgcctacgctcacagggacgttttgttctgggtacaattgtttatggtcaatccattgggtcccatctctgacaccacgtatgagtttaccgacggtctgtacgatgttctagctagagctgtgccagaatctgttggtcatgcctatttgggttgtccagaccctagaatggaagatgcccaacagaagtactggagaaccaaccttccaagattacaagaattgaaggaagaactagatccaaagaatacatttcatcaccctcaaggtgtaatgcctgct

SEQ ID NO: 93 - DNA, 1428 bp, Artificial Sequence
  Synthetic
atgggaaacacaacgtccatagctgccggtagagactgcctattatcggcagtaggcggtaatcacgctcatgtcgctttccaagatcagcttttgtatcaagtgaccgctgttgagccttacaacttgaatattccagttacccccgccgctgttacttacccacaatcagccgacgaaatcgctgccgtcgtcaaatgtgcttctgaatatggttacaaggttcaagctaggtctggtggtcactcctttggtaactacggtctgggtggtgaagatggcgctattgttgtggaaatgaagcatttcaatcaatttagtatggatgaatctacttatactgcaactatcggtccaggaattaccttgggtgacttggacaccgctttatacaacgctggtcacagagccatggcacatggtatctgtccaaccatacgtactggtggccacttgaccatgggtggtctgggtcctacagctagacaatggggtttagcattagatcatgtcgaagaggtcgaagttgttttggctaacagctctattgtcagagccagtaacacacagaatcaagatattttgttcgctatcaagggtgccgctgcttccttcggtattgttactgagtttaaagtaagaactgaagccgctccaggtgttgcagtccaatactccttcacttttaacctaggaacgccagctgaaaaggcaaagcttgttaaagactggcaagccttcatcgctcaagaagatttgacttggaagttctattctaacatgaatatatttgacggccaaatcattttggaaggtatctacttcggtagtaaggaagagtacgatgctttaggtttagaaaagagatttccctcatctgaagctggtaccgtgttggttttgaccgattggttgggtatggtcggccacggtctggaagatgtgattctaagattggttggtaacaccccaacttggttctacgcaaaatcattgggattcactccaagagctttgatacctgactcagctattgacgaatttcttaattacatccacgaaaacacgcctggtacagtatcctggttcgtcactctatctttggaaggtggtgccattaacaaggtcccaggcgatgctactgcctatggccaccgtgatgtgttattctgggttcagatttttatgatcaacccattgggtccagtttctcaaaccacttatggtttcgctgacggattatatgacgttttggcaaaggctgtaccaaactcggctggacacgcctacttaggttgtcccgatccaagaatgccaaatgctcaacaagcttattggaggtctaatttgcccagattggaggaattgaagggtgaactggatccaaaagacatttttcataacccacaaggtgttatggttgtctcc

SEQ ID NO: 94 - DNA, 1419 bp, Artificial Sequence
  Synthetic
atgggcaatacgacatccattgcaggtagagattgtcttataagcgccctaggtggaaactcggctttggctgctttccctaacgagttactgtggactgctgacgtccatgaatacaatttgaacttgcccgttactccagccgctatcacctatccagaaaccgctgaacaaatcgctggtattgtgaaatgcgcctctgattacgactataaggttcaggcacgttctggtggtcactcatttggtaattacggtttgggtggtgccgatggagctgttgtagtcgacatgaagcacttcactcaatttagtatggatgacgaaacctacgaagctgtcatcggtccaggtacaactttaaacgacgttgatattgaattatataacaatggcaaaagagccatggcacatggtgtttgtccaactatcaagaccggaggtcacttcaccattggtggtttgggtcctacagctagacaatggggtttggctctggaccacgtcgaggaagtagaagttgtcttggcaaactcttccattgtgagggcctctaacactcaaaatcaagatgttttctttgcagttaagggtgctgctgctaacttcggtatagtgaccgagtttaaagttagaacggaaccagctccaggcttagctgtccagtactcctatactttcaacttgggttcaactgctgaaaaggctcaattcgttaaggattggcaatcattcatctctgctaagaatcttactagacaattttacaacaacatggtcatttttgacggtgatatcattttagaaggtttatttttcggcagtaaggaacaatacgacgccttgggtttggaagatcattttgcaccaaagaaccctggtaacattttggtactaaccgactggttgggaatggttggtcacgccctagaagatacaatattgaaattggttggcaatactccaacctggttctacgctaaatctttgggtttcagacaggataccttgattccatccgctggtatcgacgaatttttcgaatatattgctaatcatactgctggtaccccagcttggttcgtcaccttaagcctagagggtggtgccatcaatgatatcgctgaagacgctactgcctacgcacatagagatgtcttattctgggtccaactgtttatggttaaccctttgggtcccatatctgatacaacttacgaatttacagacggtctgtatgacgttctagcacgtgctgtaccagagtctgtcggccacgcttacttaggctgtcccgacccaagaatggaagacgcacaacaaaagtattggagaaccaacctaccaagattgcaagaattgaaggaagagttggacccaaagaacacgtttcaccatccacagggtgttatgcctgca

SEQ ID NO: 95 - DNA, 1419 bp, Artificial Sequence
  Synthetic
atgggtaatacgacttccatagccggaagggactgcctaatctctgctttgggtggtaactcggctctggcagtcttccctaacgagttattgtggaccgctgatgttcacgaatacaatttgaacttgccagttactccagccgctattacctatcccgaaacagctgcacagattgctggcgtagtcaaatgtgcctcagattacgactacaaggtgcaagctagatctggtggtcatagctttggtaactatggtttaggaggtgctgatggcgcagttgttgtcgacatgaagcacttcactcaatttagtatggatgacgaaacttacgaagctgttatcggtccaggtaccaccctaaatgatgttgatatcgaattgtataacaatggtaagagagctatggcacatggtgtttgtccaacaattaaaactggaggtcacttcaccattggcggtttaggtcctactgccagacaatggggtcttgctttggaccatgtcgaagaagtagaggtcgttcttgctaactcttctatcgttcgtgcttccaacactcaaaaccaagatgtgttctttgccgtcaagggtgctgctgccaacttcggtattgtaacagaatttaaagttagaactgaaccagctccaggtttagccgtccagtactcttataccttcaatttgggttccacggctgaaaaggctcaattcgttaaggactggcaatccttcatatctgccaagaatttgaccagacaattttacaataacatggttatctttgacggagatattatattggagggtctatttttcggtagtaaggaacaatacgacgctctgggcttagaagatcactttgctccaaaaaacccaggtaatatcttggtattgaccgattggttgggtatggtcggtcatgcccttgaagatacaattttgaagctggttggtaacactccaacttggttctacgcaaagtccttaggtttccgtcaagacacgttaattccttcagccggcatcgatgaatttttcgaatacatcgctaaccacaccgctggtactcctgcttggttcgtcaccttgagcttggaaggcggtgccattaacgatgtcgccgaggacgcaacggcttacgctcacagagatgttttgttctgggtccaattattcatggtgaatccagtgggtcctatatctgacactacttatgaatttactgatggtttgtacgacgttctagctagagcagtccctgagagcgtgggtcatgcttatttgggttgtccagacccaagaatggaagatgcccaacagaaatattggaggacaaatttacccagattgcaagaattaaaagaggaattggatccaaagaacacattccaccatccacagggtgttatgcccgct

SEQ ID NO: 96 - DNA, 1428 bp, Artificial Sequence
  Synthetic
atgggcaatacaacgtccattgccgctggtcgtgactgcttgatcagcgctgttggaggtaacgcagctcacgtggcctttcaggatcaacttttatatcaagctaccgcagtcgatgtttacaacttgaacatacccgtcactccagctgccgtaacttaccctcaatcagctgacgaggttgctgctgttgtcaagtgtgcctcggaatacgattataaagtccaagctagatctggtggtcattctttcggtaattacggtctaggtggtcaaaatggagctattgttgtcgacatgaagcacttcagtcaatttagtatggacgaatcaacctatactgcaaccatcggcccaggtatcactctgggtgatttagataccgaattgtacaacgctggtcatagagcaatggctcacggtatttgtccaacaataagaactggtggtcacttgactatcggtggtttgggtccaacagccaggcagtggggtctggctttagaccatgttgaagaggtagaagttgtgttggctaactcttccattgttagagcctctgaaacgcaaaaccaagatgtcttgttcgcagtaaagggcgctgctgcttcctttggtattgttaccgaatttaaagttagaactgaacaagctcctggcctagctgtccagtattcctacaccttcaatttgggtaccccagctgagaaggccaagttattaaaagactggcaagctttcatcgcccaagaagacttgacctggaagttctactccaatatggttattttcgatggtcaaatcattttggaaggaattttctttggttctaaggaagaatatgatgccctggatcttgagaagagatttccaacttctgaacctggtactttgttggttttaacggactggcttggtatggtaggtcatagcctggaagacgtcatattaaggctagttggtaacaccccaacttggttttacgctaagtctttgggcttcactccaagaaccttgatccctgacagcgctatagatagattcttcgactatattcacgaaactaacgctggtaccttggcatggtttgtgacgctttcattggaaggtggtgctattaatgccgtgccagaagatgcaaccgcctacggtcatcgtgatgttttgttttgggttcaaatcttcatggtcaaccccttgggaccaatttctcaaactatctacgatttcgctgacggactatacgacgtgttggcacaagccgtaccagaatcggctgaacacgcttacttaggatgtccagatcctaaaatgccagacgcccaacgtgcttattggagaggtaacttaccaagactggaggaattgaaaggagagtttgatcccaaggacacatttcacaacccacagggtgtttctgtcgccgtc

SEQ ID NO: 97 - DNA, 1428 bp, Artificial Sequence
  Synthetic
atgggcaacaccacgagcatcgctgccggtagagattgtttaatatctgctgttggaggtaatgcagctcacgtcgcctttcaggaccaactgctttaccaagctactgctgtggaaccttataacctaaatattccaatcaccccagccgctattacatacccccaatcggctgatgagatcgcagcagttgtaaagtgcgcttcagaatatggttacaaagtccaagctcgttccggtggtcattctttcggtaactacggtttaggtggtgaagacggtgctattgttgtcgaaatgaagcacttcagtcaattttccatggatgaatctacttatattgccactatcggcccaggtattacattgggagacttggataccgaattatacaatgttggtcatagagctatggcccacggtatctgtccaactattagaaccggtggtcatttgactgttggaggtttgggtcctaccgctaggcaatggggcctggccttggatcacgttgaggaagtcgaagtcgtattggctaactcttccatagttagagcatcagacactcagaaccaagacatcttcttcgctattaaaggtgctgctgctagctttggtatagtgacagaatttaaggttagaaccgagcaagccccaggtctagccgtgcaatactcttacactttcaacttgggtacaccagctgaaaaggccaagttggttaaggactggcaggctttcattgctcaagaaaatctgtcatggaaattctactctaatatggtcgtattcgatggccaaatcatcttagaaggtttgtactttggctccaaggaagaatatgatgctcttggtcttgaacaacgtttccccccatctgaagctggtaacgttctagtcttgactgattggttgggtatggttggtcatgagttagaagatactattttgagattggtaggtaacacccctacttggttctacgctaaaagcttgggatttaccccaagagccctgattccagactccgcaatagatgacttattcaactatattcacgagaataacccaggtaccttggcatggttcgtcacactttctttagaaggtggtgcaatcaacaccgttcctgaacacgctactgcctatggacatagagatgttttgttttgggtccaaatttttgttatcaatccattgggtcccgtcagccaaacgacttacggttttgctgatggtatgtatgacgtgcttgcccaagctgttccagaaagtgctggtcatgcttacttgggttgtccagatccacgtatgccaaacgcccaacaagcttactggagatctaatttgcctagattagaagaattgaagggcgacctagacccaaaaggtatcttccacaatccacaaggtgttatggtagtctcc

SEQ ID NO: 98 - DNA, 1428 bp, Artificial Sequence
  Synthetic
atgggtaacactacgtcgatcgcagctggacgtgattgcctattgtccgctgttggtggcaatcatgcccacgtagctttccaggaccaacttttgtatcaagccacagctgtcgaaccatacaacttaaacatacctgtgactccagctgccgttacctacccccaatctgctgatgaggtcgcagctgttgttaagtgtgctgccgactatggttacaaagtccaagctagatcaggtggtcacagttttggtaattacggtttgggtggtgaagacggtgctattgttgtagatatgaagcatttcgatcaatttagcatggatgaatctacctacactgccaccatcggcccaggtattactctgggcgacttggataccgctttatataatgccggtcacagagctatggcacatggtatctgtccaactattagaacaggcggtcacttgaccattggtggtttgggtcctacggctaggcaatggggattggcactagaccacgtcgaagaagttgaggttgtcctggctaactcctctatagtcagagcctctgacactcagaaccaagaaattttattcgctgttaagggtgctgccgcttccttcggtatcgtcactgaatttaaagttagaaccgaagaagctccaggattggcagtccaatacagcttcaccttcaaccttggtactgccgctgaaaaggctaagttggtgaaagattggcaagcttttatcgcccaggaagacttaacgtggaagttttattctaacatgaacattatcgatggtcaaattattctggagggtatctacttcggttcgaaagctgaatacgacgcattgggattggaagagaagtttccaacatcagaacccggtactgtgcttgtattaactgactggttgggtatggttggtcacggtttagaagatgttattttgcgtttggttggaaatgctccaacttggttttatgcaaagtcactaggtttcgctccaagagctttaatacctgatagtgcaattgatgacttcttcgaatatatccataagaataacccaggtacagtctcttggttcgtcaccttgtccttggagggtggtgccatcaataaagtaccagaagatgccactgcttacggtcatagagatgttctattctgggttcaaatttttatgatcaatccattaggtccagtttctcaaacgatctacgatttcgctgacggcttgtatgacgttctggctaaggccgtacctgaatccgctggtcacgcatacctaggttgtcccgacccaagaatgcctaacgctcaacaggcctactggaggaacaacttgccaagattggaagaattgaagggtgacttagatccaaaagatattttccataatcctcaaggagtgatggtcgtgagc

SEQ ID NO: 99 - DNA, 1422 bp, Artificial Sequence
  Synthetic
atgggtaatacgacttccatcgccggccgtgactgcttggttagtgcactaggtggaaacgctggtttagtggctttccaagatcagcttttgtatcaaaccacagctgtacacgagtacaacttgaacattccagtcacccctgccgcagttacttacccagaaactgctgaacaaatagctgccgtcgtgaaatgtgcttctgaatatgattacaaggtccaagctagatctggtggacattcgtttggtaattacggtctaggtggtgctgacggtgctgtagttgttgatatgaagcacttctcacaattttccatggacgatcagacatatgaagcagttatcggtcccggtaccactttaggtgacgtcgacaccgaattgtacaacaacggcaagagagctatggcccatggtatttgtccaacaattagtactggtggacacttcactatgggtggtctgggtccaaccgccagacaatggggtttggctttggatcacgttgaagaggttgaagtcgttttggcaaattcttctatcgttagggcttccaacacccaaaatcaagaagtcttctttgctgtcaaaggtgccgctgcctcatttggtatcgttacagagttcaaggtcagaactcaacctgctccaggcttagcagtacagtacagctatacgtttaatattggttcgtctgctgaaaaggcccaattcgttaaagattggcaatcattcattagtgctaagaaccttactagacaattctacaccaacatggtaatcttcgatggtgacataattttggaaggattatttttcggttcccaagaacaatatgaagctttgggtctggaagacagatttgttccaaagaaccctggaaatattttggtgttgacggattggctgggtatggttggtcatgcccttgaagacactatcttaagattggtcggtaacactccaacttggttttacgctaaatctttgggattcaccccagacactttaattccagcttccggtatcgatgaatttttcgattacatagaaaaccataaggcaggcaccttgacgtggttcgtcactttgtctctggaaggtggtgctatcaatgatgtcccagaggacgctacagcctacggtcatagagatgttttgttctgggttcaaatttttatggcttctcccaccggtcctgtctcctctaccacctatgacttcgccgatggtctatataatgttttaactaaggctgtaccagagagcgaaggtcacgcttacttaggttgtccagaccctaagatggccgatgctcagcaaaaatactggcgtcaaaacttgccaagattggaagaattgaaggcaactttagacccaaaagataccttccacaatccccaaggtatcttgccagct

SEQ ID NO: 100 - DNA, 2082 bp, Artificial Sequence
  Synthetic
atgtggttgtctacaatgaatggttcagccagtagacgtagcgatcccgtcagcagaaaaatcgtttgcgacggccatgcttctgcacacgaggtgaggactgacaacgaagctgctagagatgtaccttcgagaaccgctgtcaacaaggaaagaaagcagggttccggtccaccaggagccatgcaaagaggttttcacgctgcccataagccaaatgaaatggttccacaagacggtcctcttggtagaactgctcaattattccgtctggcaccagcttgtcaatcagaaccaacgagagctccaggtcaaccatctgatctaagattgcgtcaaattcccttggcaaccgagcaagctgccagaactttggctaggatgcgtccagcaagattcacatttccttatggaagagccgctgaagatgattgttacttaaaaaaggaagacgaaggtcacgaccagtctcatccaacctccgtcttggttggtgttccccctttcactagacgttgtgctgctgctgaaaccttcaaagatactagagctagagctccaggtacacaaccaactgataccacttctacaggtgcctccccatcatggaccttatctccactattgtccttgtctgctactgacgacagtgttccttccaagatgggcaacggtcaatctaccccacttcaacaatgtttgaatactgtttgcaacggtaggctgggttgtgtcgcctttccatcggatgccttataccaggctgcttgggtcaagccatataacttggacgtacctgttaccccaatagctgtgtttaagcctagttccacggaggatgttgctggtgccattaagtgtgctgtcgcttctaacgttcacgtgcaagccaagtctggtggtcattcgtacgctaacttcggattgggtggacaagatggtgaactaatgattgatttggcaaatttacaggacttccacatggacaaaacatcctggcaagctactttcggtgccggttacagattgggtgatttagataaaaagttacaagcaaatggcaacagagctatcgcacacggcacatgtccaggtgttggtattggaggtcatgccactatcggcggtctaggcccaatgagccgtatgtggggttccgctttggaccacgtcttgtctgtccaagttgtcaccgctgacggtagtatcaaaaacgcctctgaatcagaaaactctgatctgttttgggccttgagaggtgctggtgcttcatttggagttattactaagttcactgttaagacccatccagctccaggttccgttgtacaatatacatacaaaatctctttgggtagccaggcacaaatggcccctgtttacgctgcctggcaagctttagctggtgaccccaagctggacagaagattttccacattgttcatcgcagaacctcttggtgctctgattaccggaactttctatggtactaaagctgagtacgaagctactggtatagctgctagattgccttctggtggtaccttggatttgaagcttttagattggttgggtagtttggctcacatagctgaagtagttggtttgaccttgggtgacattccaacgtccttttacggtaagtcgctagctttgagagaagaagacatgttagaccgtacttctattgatggtttgttcaggtatatgggtgacgccgatgcaggcaccctattatggttcgtcatcttcaattccgagggtggtgccatggctgatactccagctggtgccaccgcctacccacatagagataaattaatcatgtatcaatcatacgttattggtataccaaccctgactaaggccaccagagattttgctgatggtgtccacgaccgtgtgagaatgggtgctccatctgctaacagtacgtatgcaggttacatcgatagaaccttgtccagagaagctgctcaagaattttactggggcgctcaactgcctagattgagagaagtcaagaaagcatgggacccaaaggacgtctttcacaacccacaatccgttgatccagcagag

SEQ ID NO: 101 - DNA, 1428 bp, Artificial Sequence
  Synthetic
atgggcaatacaacttccattggtgtagtgagggattgtttgacgtctgctgtcggtggtgttgcagcccatgtcgctttccaggacaccctattataccaaacctcagctgttaaaccatataaccttaacgtccctgttactcccgccgctgttacttacccacaaagtgctaatgaagtcgctgctatcgttaagtgcgcatcggattatgactacaaggtacaagctcgttccggtggacacagctttggtaacttcggtttaggtggacaaaacggtgccatagttattgacatgaaacacttttctcaattctccatggatgagtctaccttcatcgccactattggcccaggcaccactttgggtaatctggatacagaattgtacaacgctggtaatagagctatggctcatggtatatgtccatcgatcagaactggtggtcacttgaccgttggaggtttgggtcctacctctcgtcaatggggtctagctctggaccacgtcgaagaggtggaagttgtacttgctaactcttcagtcgtcagagcctctgacacgcagaaccaagatgttttatttgctatcaagggtgcagccgcatccttcggtatcgttactgaatttaaggtcagaacagaagaagctccaggtttggccgttagatattcctacagcttcaacttgggtactccagctgaaaaagcaaagttggctaaggattggcaagcctacattgcccaagaaaacttaacgtggaaattctctagtaacttgattattttcgacggtcaaattatccttgagggaatatttttcggtagcaaggaagaatacgacaagttaaatttggaaaagaagtttccaacttcagaacctggtaccgtcttggtcattacgaattggttgggtatgatcggacatgctttggaagataccatcctaagacttatcggtgattcacccacttggttctatgctaaatctttgggttttactccaaacacactaatctttgactctaccattgacgaatttttcgattacatacacaaggctaacgctggtacattagcttggtccgttatgttgtctttggaaggtggtgccataaatgctgttccaaaaaatgctactgcatacggtcatagagatgtattattctgggttcaaattttcgttgtgaatcctcttggaccaatttcccaaaccacttatggttttaccgatggtttgtataacatcttggccagaggtgttccagagtccgcaggtcatgcttacttaggttgtccagatcccaagatgccagacgctcaaagagcatactggagaaataactatccacgtctggaggaattgaaaagagacttggatcctaaggacatttttcacaacccacagggcgtcagagtcgcttct

SEQ ID NO: 102 - DNA, 1422 bp, Artificial Sequence
  Synthetic
atgggcaacactacatcaattgctgccggtagagattgcctagtaagcgcagtcggtccagctcatgttaccttccaggacgcccttctgtaccaaactacggctgtcgatccttataatttaaacatcccagtgacccccgctgctgttacttacccacaatcggctgaagagatagccgctgttgtcaaatgtgcttctgactatgattacaaggttcaagctaggtctggtggacactcctttggtaactacggtttgggtggtcaaaatggagccattgtagttgacatgaagcacttctctcaatttagtatggatgaatctaccttcgtcgcaactattggtccaggtacaaccttgggcgacttggatactgaattgtataacgcaggcggtagagctatggcccatggtatctgtcctacaatccgtactggtggtcacttaactgtcggtggtttgggtccaaccgctagacaatggggtctggccttagatcacattgaagaagttgaagtggttttggctaattcctcgatagtgagagctagcaacactcagaaccaagacatcttgttcgccgttaagggtgctgctgcttcatttggtattgtcaccgagtttaaagttagaacccaagaagcaccaggactagctgttcaatacagtttcaccttcaatttgggttccccagctcagaaagccaagttggtcaaggactggcaagcattcattgcccaagaaaacttatcttggaagttctactctaatttagtcatctttgacggtcaaattattttagaaggtatctttttcggatccaaggaggaatatgatgaattggacttggaaaaaagatttcccacttctgaaccaggtacagttctggttttaacggattggttgggaatgatcggccatgcacttgaggatactattttgaagttggtcggtgacacacctacgtggttttacgctaagtcccttggcttcactccagataccttgatcccagattcggctattgatgatttcttcgactatattcataagactaacgctggtactctggcctggtttgtgaccttatctttggaaggtggcgctataaactccgtttcagaagatgctaccgcttatggtcacagagatgtcttgttttggttccaagttttcgttgtcaatcctcttggtccaatctctcaaacaacatacgacttcactaatggtttgtacgacgtattggctcaggccgtgcctgaaagcgctggtcatgcttaccttggttgtccagatccaaaaatgccagacgctcagcgtgcttactggagaagtaacttacccagattggaggatctgaagggtgatcttgacccaaaggacacctttcacaaccctcaaggtgttcaagtcggtcca

SEQ ID NO: 103 - DNA, 1422 bp, Artificial Sequence
  Synthetic
atgggcaataccacatctatcgctgccggtagagactgtctggtcagtgctgttggtcctgcacacgtgacgtttcaggatgctttgctttaccaaactactgctgttgatccctataacttaaacataccagtaaccccagccgctgtcacttacccacaatccgctgaggaaattgccgctgttgtgaagtgcgcttcagactacgattataaagtccaagctaggtctggaggtcatagcttcggtaactacggtctaggtggtcaaaatggtgcaatcgttgttgaaatgaagcacttctctcaattttccatggacgaatcgaccttcgtcgccactattggcccaggtacaacattgggtgatttagataccgaattgtataatactggtggccgtgctatggcccatggtatttgtccaactatcagaaccggtggtcacttgaccgttggtggattgggtcctactgcaagacaatggggtttagctcttgatcatatcgaagaagttgaggtcgtcttggctaactcttccattgttagagctagcaacactcagaaccaagacattctatttgctgttaaaggagccgctgccagcttcggtatagtcaccgaatttaaggttagaacacaggaagctccaggtttggctgtacaatacagtttcaccttcaatttgggctcagcagctcaaaaggcaaagttggtcaaagactggcaagccttcatcgctcaagaaaatttatcttggaaattttactctaacctagttatttttgacggacaaattatcttggaaggtatcttcttcggttccaaggaggaatacgatgaactagacttagaaaagagattcccaacttctgaaccaggtaccgtgttggttttaactgattggttgggtatgatcggtcacggtctggaagacactatattgaagttagttggtgatacccctacttggttctatgcaaagtccttgggttttacgccagatactttgatacccgattctgccattgacgattttttcgattatattcataagacaaatgctggaaccttggcttggtttgtaacgctatctttggaaggtggtgctataaactctgtctcggaagacgcaacagcttacggtcacagagatgtcctgttttggttccaagtgtttgtagtcaaccctttgggtccaatttcccagaccacttacgacttcaccaatggtttatacgatgttcttgctcaagccgttccagaatcggccggccacgcttatttgggttgtccagaccctaaaatgcccgacgcacaacgtgcttactggaggtccaacctaccaagattggaggacttaaagggtgacctagacccaaaggatacttttcataacccacaaggtgtccaagttggacca

SEQ ID NO: 104 - protein, 473 aa, Purpureocillium lilacinum
MGNGQSTPLQQCLNTVCNGRLGCVAFPSDALYQAAWVKPYNLDVPVTPIAVFKPSSTEDVAGAIKCAVASNVHVQAKSGGHSYANFGLGGQDGELMIDLANLQDFHMDKTSWQATFGAGYRLGDLDKKLQANGNRAIAHGTCPGVGIGGHATIGGLGPMSRMWGSALDHVLSVQVVTADGSIKNASESENSDLFWALRGAGASFGVITKFTVKTHPAPGSVVQYTYKISLGSQAQMAPVYAAWQALAGDAKLDRRFSTLFIAEPLGALITGTFYGTKAEYEATGIAARLPSGGTLDLKLLDWLGSLAHIAEVVGLTLGDIPTSFYGKSLALREEDMLDRTSIDGLFRYMGDADAGTLLWFVIFNSEGGAMADTPAGATAYPHRDKLIMYQSYVIGIPTLTKATRDFADGVHDRVRMGAPAANSTYAGYIDRTLSREAAQEFYWGAQLPRLREVKKAWDPKDVFHNPQSVDPAE

SEQ ID NO: 105 - protein, 473 aa, Aspergillus tubingensis
MGNTTSIAGRDCLISALGGNSALAVFPNQLLWTADVHEYNLNLPVTPAAITYPETAEQIAGIVKCASDYDYKVQARSGGHSFGNYGLGGTDGAVVVDMKHFNQFSMDDQTYEAVIGPGTTLNDVDIELYNNGKRAMAHGVCPTIKTGGHFTIGGLGPTARQWGLALDHVEEVEVVLANSSIVRASNTQNQDVFFAVKGAAADFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDQFFEYIANHTAGTPAWFVTLSLEGGAINDVAEDATAYAHRDVLFWVQLFMVNPLGPISETTYEFTDGLYDVLARAVPESVGHAYLGCPDPRMENAPQKYWRTNLPRLQELKEELDPKNTFHHPQGVIPA

SEQ ID NO: 106 - protein, 473 aa, Aspergillus niger
MGNTTSIAGRDCLISALGGNSALAAFPNQLLWTADVHEYNLNLPVTPAAITYPETAEQIAGIVKCASDYDYKVQARSGGHSFGNYGLGGTDGAVVVDMKHFNQFSMDDQTYEAVIGPGTTLNDVDIELYNNGKRAMAHGVCPTIKTGGHFTIGGLGPTARQWGLALDHVEEVEVVLANSSIVRASNTQNQDVFFAVKGAAADFGIVTEFKVRTEPAPGLAVQYSYTFNLGSTAEKAQFVKDWQYFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIANHTAGTPAWFVTLSLEGGAINDVAEDATAYAHRDVLFWVQLFMVNPLGPISETTYEFTDGLYDVLARAVPESVGHAYLGCPDPRMENAPQKYWRTNLPRLQELKEELDPKNTFHHPQGVIPA

SEQ ID NO: 107 - protein, 513 aa, Phaseolus vulgaris
MDPIEDAILQCLSLHSDPSHPISGVTYFPNTPSYIPILHSYIRNLRFTSPSTRKPLFIVAPTHISHIQASIICCKSFQLQIRIRSGGHDYDGLSYVSQSPFAIMDMFAMRSVEVNLEDETVWVDSGSTIGELYHGIAERSKVHGFPAGVCHSVGVGGHFSGGGYGNMMRKFGLSVDHVLDAVIVDAEGRVLDRKKMGEDLFWGIRGGGGASFGVIVSWRIKLVPVPEVVTVFRVLKTLEQGATDVVHRWQYVADNIHDDLFIRVVLSPVKRKGQKTIRAKFNALFLGNAQELLRVMSDSFPELGLVGEDCIEMSWIDSVLFWDNFPVGTSVDVLLQRHDTPEKFLKKKSDYVQQPISKTGLEGVWNKMMELEKPVLTLNPYGGRMGEISEMEIPFPHRAGNLYKIQYSVNWKEEGEDVANRYLDLIRMLYDYMTPYVSKSPRSSYLNYRDVDIGVNGPGNATYAEARVWGEKYFKRNFDRLVEVKTRVDPSNFFRYEQSIPSLAASSLGIMSE

SEQ ID NO: 108 - protein, 506 aa, Dactylellina haptotyla
MVHNILLLGLMPLLVRASPLPIYHNYPPQSTINDCLQAADVPAILQSSASFDALSQPLNSRLKSKPAVITIPTTALHVSSAVKCAAQFKLKVTPRGGGHSYNAQSLGDGAVVIDMQQFHDVVYDSKTQLARIGGGARLGNVAQKLYDQGKRAMPHGTCPDVGIGGHSAGGFGWTSRQWGITVDHIDEVEVVTADGSIRRANKDQNSDLFWALRGAAPSFGVITNFWFSTLEAPDSNVIYSYKFTGLSLDEISTALLEVQKFGQTAPKEVGMLIQILDNGSGFRLYGTYYNTTRQQFDNLFGQLLQRLPSPGNSAEVSVKGWIDSLIFASGGSKGLTVPELGGTNQHSSFYTKSLMTAQDYPLTLDSIKSVFKYAMNQGRAATERGLPWMVFISLLGGRYSTLPTPSAASDNSFYGRNTLWAFSFTAYLGNVTEQSNRDSIYFLNGFDTSVRRSVDTAYINGHDTEYSREEAHRLYYGDKYQRLSVLKKQWDPEQVFWYPQSIDPAN

SEQ ID NO: 109 - protein, 476 aa, Aspergillus fumigatus
MGNTTSIAAGRDCLLSAVGGNHAHVAFQDQLLYQATAVEPYNLNIPVTPAAVTYPQSADEVAAVVKCAADYGYKVQARSGGHSFGNYGLGGEDGAIVVDMKHFDQFSMDESTYTATIGPGITLGDLDTALYNAGHRAMAHGICPTIRTGGHLTIGGLGPTARQWGLALDHVEEVEVVLANSSIVRASDTQNQEILFAVKGAAASFGIVTEFKVRTEEAPGLAVQYSFTFNLGTAAEKAKLVKDWQAFIAQEDLTWKFYSNMNIIDGQIILEGIYFGSKAEYDALGLEEKFPTSEPGTVLVLTDWLGMVGHGLEDVILRLVGNAPTWFYAKSLGFAPRALIPDSAIDDFFEYIHKNNPGTVSWFVTLSLEGGAINKVPEDATAYGHRDVLFWVQIFMINPLGPVSQTIYDFADGLYDVLAKAVPESAGHAYLGCPDPRMPNAQQAYWRNNLPRLEELKGDLDPKDIFHNPQGVMVVS

SEQ ID NO: 110 - protein, 474 aa, Aspergillus arachidicola
MGNTTSIAGRDCLVSALGGNAGLVAFQSQPLYQTTAVHEYNLNIPVTPAAIAYPETAEQIAAVVKCASEYDYKVQARSGGHSFGNYGLGGTDGAVVVDMKHFNQFSMDDQTYEAVIGPGTTLGDVDTELYNNGKRAMAHGICPTISTGGHFTMGGLGPTARQWGLALDHVEEVEVVLANSSIVRASNTQNQEVFFAVKGAAASFGIVTEFKVRTQPAPGLAVQYSYTFNLGSSAEKAQFVKDWQSFISAKNLTRQFYTNMVIFDGDIILEGLFFGSKEQYEALGLEERFVPKNPGNILVLTDWLGMVGHALEDTILRLVGNTPTWFYAKSLGFTPDTLIPSSGIDEFFEYIENNKAGTSTWFVTLSLEGGAINDVPADTTAYGHRDVLFWVQIFMVSPTGPVSSTTYDFADGLYNVLTKAVPESEGHAYLGCPDPKMANAQQKYWRQNLPRLEELKATLDPKDTFHNPQGILPV

SEQ ID NO: 111 - protein, 483 aa, Aspergillus flavus
MNPSIPSSSMGNTTSIAGRDCLVSALGGNAGLVAFQNQPLYQTTAVHEYNLNIPVTPAAITYPETAEQIAAVVKCASQYDYKVQARSGGHSFGNYGLGGTDGAVVVDMKYFNQFSMDDQTYEAVIGPGTTLGDVDVELYNNGKRAMAHGVCPTISTGGHFTMGGLGPTARQWGLALDHVEEVEVVLANSSIVRASNTQNQEVFFAVKGAAASFGIVTEFKVRTQPAPGIAVQYSYTFNLGSSAEKAQFIKDWQSFVSAKNLTRQFYTNMVIFDGDIILEGLFFGSKEQYEALGLEERFVPKNPGNIMVLTDWLGMVGHALEDTILRLVGNTPTWFYAKSLGFTPDTLIPSSGIDEFFEYIENNKAGTSTWFVTLSLEGGAINDVPADATAYGHRDVLFWVQIFMVSPTGPVSSTTYDFADGLYNVLTKAVPESEGHAYLGCPDPKMANAQQKYWRQNLPRLEELKETLDPKDTFHNPQGILPA

SEQ ID NO: 112 - protein, 506 aa, Aspergillus nomiae
MARVPALFLGLSSVCLLVFFCSLINPTTPLHQHGKQTSIAGRDCLVSALGGNTGLVAFQDQLLYQTTAVHEYNLNIPVTPAAITYPETSEQIAAVVKCASEYDYKVQARSGGHSFGNYGLGGADGAVVVDMKHFTQFSMDEQTYEAVIGPGTTLGDVDTELYNNGKRAMAHGICPTISTGGHFTMGGLGPTARQWGLALDHVEEVEVVLANSSIVRASNTQNQEVFFAVKGAAASFGIVTEFKVRTQPAPGLAVQYSYTFNLGSSAEKAQFVKDWQSFISAKNLTRQFYTNMVIFDGNIILEGLFFGSKEQYEALGLEDRFVPKNPGNILVLTDWLGMVGHALEDTILRLVGNTPTWFYAKSLGFTPDTLIPSSGIDEFFDYIENNKAGTLTWFVTLSLEGGAINDVPKDATAYGHRDVLFWVQIFMASPTGPVSSTTYDFADGLYNVLTKAVPESEGHAYLGCPDPKMADAQQKYWRQNLPRLEELKATLDPKDTFHNPQGILPA

SEQ ID NO: 113 - protein, 427 aa, Aspergillus niger
MTTKCRHAVEATALGTMVAYLIRNNPRRPSSTNANVLDTGLGGADGAVVVDMKHFTQFSMDDETYEAVIGPGTTLNDVDIELYNNGKRAMAHGVCPTIKTGGHFTIGGLGPTARQWGLALDHVEEVEVVLANSSIVRASNTQNQDVFFAVKGAAANFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIANHTAGTPAWFVTLSLEGGAINDVAEDATAYAHRDVLFWVQLFMVNPLGPISDTTYEFTDGLYDVLARAVPESVGHAYLGCPDPRMEDAQQKYWRTNLPRLQELKEELDPKNTFHHPQGVMPA

SEQ ID NO: 114 - protein, 475 aa, Neonectria ditissima
MGHGQSTPGENCLNAICGNRTDCVSYPLDPLFHIAWARPYNLQVPVTPAAVLRPDNAQDVADAVKCANENGIKVQARSGGHSYGNFGLGGDDGALVLDLVNLQGFTMDEENWHAAVGAGIRLGKLDEHLHKNGGRAMAHGTCPGVGIGGHATIGGLGPMSRMWGSALDHVVEVEVVTADGSIQRANETENSDLFWALRGAGASFGIITEFVVRTHPEPGDVIEYTYSLTFGSQAEMAPIYQEWQEFIGDPDLDRRFSSQFIAQPLGAIITGTFYGTEDEYRESGIPDKIPGGGSDGVGILVTDWLGSLGHQAERSGLAISDLPSPFYSKSLAFRKEDLLPKEGITELFQYLNDADHGTLIWVVIFNSEGGAMGDTAANATAYPHRDKTMMYQSYVIGIPEVSGTARRFLEGVHAKIQDAAPGANSTYAGYIDTELGRAEAQEVYWGSQLPQLRKIKKDWDPKDRFSNPQSVQAAR

SEQ ID NO: 115 - protein, 516 aa, Unknown
  Pseudogymnoascus sp. VKM F-4518
MRVVGKMGALQSTLEKSIKAALAGDDDLYAVPGKPFYQIQHVKPYNLSIPIEPAAITYPKTTAQVAAIIKCAVAANLKVQARSGGHSYANYCIGGVSGAVVIDLKHFQRFSMDRTTWQAAVGAGTLLGNLTKRMHEAGNRAMAHGTCPQVGIGGHATIGGLGPSSRLWGTALDHVEEVEIVLADSTIKRCSATQNPDIFWAVKGAGASFGVVTEFKLRTEPEPSEAVHFSYSFTVGSYASLAAVFKSWQSFVADPGLTRKFSSEVIITEIGMIISGTYFGSQAEYDALDMKSQLRGDSVAKIIVFKDWLGLLGHWAEDVGLRIAGGLPAPLYAKTLTFNGANLIPDEVIDKLFAYLDKVEKGALVWFVIFDLAGGAVNDIAQDATSYAHRDALFYLQSYAVGLGNVSQTTKDFLTGINTTITNGMPEGGDFGAYPGYVDLELPNGPHAYWRTNLPRLEQIKALVDPNDVFHNPQSYLCILFLLNLLNRALAWAPVGTVQPFQVLRYSIDTGPLVLL

SEQ ID NO: 116 - protein, 478 aa, Pyrenophora tritici-repentis
MGQGSSGVQSNPLEDCLKVATSPLGSYAFHDKLLFQLTDVKPYNLDYPVNPIAVTYPGSTKEVAQIIKCATTYDKKVQARSGGHSYANFALGDGDGAIVIDMQKFKQFSMDTSTWQATIGPGTLLGDVSKRLHENGNRVIPHGTSPQIGFGGHGTIGGLGPLSRMYGLTLDSIEEVEAVLANGQIVRASKTQNEDLFFAIRGAAASVAVVTEFKVRTYPEPSSSVLYSYTLQGGSVASRANAFKQWQKLTTDPSVSRKFASTFVLSEAITVVTGTFFGTQAEFDSLDITSRLPADMISNNTEVKNWLGVVGHWGESLALRAGGGIPAHFYSKSLGFKKDEIMDDATVDKLFNYIDKADKGGAVWFVIWDLEGGAISDVPTTETSYGHRDAIFFQQSYAINLLGRVKDDTHEFLNRVNSVIMESNPGGYWGAYPGYVDTALGNSSAKAYWGINSERLQTIKSWVDAGDVFHNPQSVRPK

SEQ ID NO: 117 - protein, 526 aa, Colletotrichum orchidophilum
MQPFTSLTRSPFRSAHVISCPVALDNPPSVPIIMGQKPSSPLATCLDKVCNGRSSCVGYPNDPLFQINWVKPYNLDIPVQPIAVTRPSTAEDVAGFVKCAAENNVKVQAKSGGHSYGNFAIGGTDGALVIDLVNFQNFSMDTNTWQATFGGGHKLHEVTQKLHDNGKRAIAHGTCPGVGIGGHATIGGLGPSSRMWGSCLDHVVEVEVVTADGKIQRANDKQNSDLFFALKGAGAGFGVITEFVMRTHPEPGDVVQYSYAITFAKHRDLVPVFKQWQELIFDPTLDRRFSSEFVMQELGVAITATFYGTEDEFKKTGIPDRIPKGKVSVVINNWLGDVAQKAQDAALWLSDIQSAFTSKSLAFTHNDLISEDGIQTMMDYVDSVDRGTLIWFLILDSTGGAINDVPMNATAYRHRDKVMFFQGYGVGIPTLSGKTKDFMSGVADKIRKASPNELSTYAGYVDPTLDNAQERYWGPNLPALERIKATWDPKDLFSNPQSVRPNASAKDVEPAASGGSNNSGSKGGDS

SEQ ID NO: 118 - protein, 472 aa, Unknown
  Cordyceps sp. RAO-2017
MGSGHSSGLATCLDAVCNGRHACVAYPDHLLYQASWVDRYNLDIPVHPIAVTRPSNADDVSGFVKCAAANNVRVQAKSGGHSYANYGLGGEDGELVIDLRHLQHFSMDTNTWQATIGAGHRLWDVTHKLHENGKRAVSHGTCPGVGIGGHATIGGLGPSSRMWGSCLDHVVEVEVVTADGSIRRASERENADLFFALKGAGAGFGVITEFVMKTHPEPGSVVRYTYSVNFGRHADMVDVFDQWQALISDPGLDRRFGSEIIMHAFGLVISATFHGTRDEYEASGIPDRIPRGNVSVLLDNWLGVVGNQAQDAGLWVSEVRSSFTSRSLAFRRDQLLSRDDIVRMMDFLDRTDKGTLVWFLIFDVTGGAIGDVRTDATAYAHRDKIMFCQGYAVGIPALTRKTRVFMDGLISTIRETANSTLTTYPGYVDPSLHDAQASYWGPNLPRLTEVKTKWDPQDVFHNPQSVRPSGKD

SEQ ID NO: 119 - skipped

SEQ ID NO: 120 - protein, 476 aa, Aspergillus fumigatus; identical to SEQ ID NO: 109

SEQ ID NO: 121 - protein, 477 aa, Xylaria hypoxylon
MGQSPSSLLATCLNTVCDGRTDCVAYPNNPLYQISWVNRYNLDLPVTPIAVTRPQTVQDVSAFVKCAATNNIKVQPKSGGHSYANYGGEDGALVIDLLKLQDFSMDAKTWQATIGGGTKLADVTKRLHDNGKRAISHGTCPGVGIGGHATIGGLGPTSRMWGSCLDHVVEAEVVTADGSIKRASETENRDLFFALKGAGAGFGVVTKFVMKTHPEPGSMVQYSYSLSFGKHTDMVPVFKQWQDLVSDPNLDRRFGTEFVAHELGAIITATFYGTEAEWDASGIPQRIPKGKISVIIDDWLAVISQQAEDAALYLSDIHSAFTVRSLAFTAEETLSEQTITRVMKYIDDTNRGTLLWFLIFDATGGAISDIPMNATAYSHRDKIMYCQGYGIGLPVLNQHTKDFLTGLTDTIQASMRQNLTTYPGYVDPSLANPQQSYWGPNLAMLESIKTTYDPNDLFHNPQSVRPGNKKASMTQEF

SEQ ID NO: 122 - protein, 473 aa, Aspergillus ruber
MSNNTLIQACLLAALGFNSTRVKFPEAGTIDADPYNLDLPIVPAAITVPRSTAQVANVVKCAASNGYKVQSRSGGHSYGNHGLGGTDGAIVVDLKDLKQFSMDESTYIASIGSGMLLDEVTHKLYDNGKRAMAHGVCPQVGVGGHFTIGGLGPTARQWGSSLDHVEEVEVVLANSSIVRASNTQNQDVLFAIKGAAASFGIVTEFKVRTEPAPDVAVQYTYELILGNITERARIMVDWQDFISDPDLSRKFASIMIVFEHGMLLSGDFYGSKKEFDDLGLADRFPIRKPGNVAILTDWLGMTGHAVEELALGIVGGIPLHFYAKSMAFTRDSLMSPSTFEKLFDYLDNTDKGTLLWLIYFDLQSGATSDVPNNSTAYAHRDTLYWLQSYVVNLVGPVSNTTTAFLEGINNLIAQDVPSANTRAYPGYVDPLMPNPQWRYWGSNLPKLEKIKAAIDPNDVFHNPQSVKPRKQAP

SEQ ID NO: 123 - protein, 504 aa, Verruconis gallopava
MGQSQALSAFKKDLAAALNNDDELFALPDEPFYIKDHVKRYNLDIETTPLAVTYPKTTAQVSSIVKLAKDNNLKVQAKCGGHSYANFCDPNGGIIVDLKHFQKFEIDENTWRCRVGGGTLLGDLTKRMFEPHKRAMAHGTCPTVGIGGHATIGGLGPSSRLWGAALDHVEEVEMVVANGDVIRANENENSDIFWAVKGAGASFGVITEFVVRTEPAPGRLVQYSYSFTTGSWKDMAKTFKAWQTYVSQPDLTRSFASTATITELGLTISVTYFGTDEEFDKINFAKNFPGNQTPKTIVFDDYLGAVGHWAEDVALEIISPLPAHSYTKTLTFNHCNQIPDSVIDRMFKYFEEVSKGTLVWFAIFDLAGGRVNDIPQDATAYAHRDALFYLQSYAVNPFGPVSNKSKQFLQGLNKVIRDGMAEAGENTDLGAYAGYVDLELGAGAQKAYWRTNLPRLESIKLKWDPEDVFHNPQSVRPGGNDVISTPKVVYKKAGFLARLKGCFR

SEQ ID NO: 124 - protein, 474 aa, Aspergillus parasiticus
MGNTTSIAGRDCLVSALGGNADLVAFQNQLLYQTTAVHEYNLNIPVTPAAITYPETAEQIAAVVKCASEYDYKVQARSGGHSFGNYGLGGTDGAVVVDMKHFNQFSMDDQTYEAVLGPGTTLGDVDTELYNNGKRAMAHGICPTISTGGHFTMGGLGPTARQWGLALDHVEEVEVILANSSIVRASNTQNQEVFFAVKGAAASFGIVTEFKVRTQPAPGLAVQYSYTFNLGSSAKKAQFVKDWQSFISAKNLTRQFYTNMVIFDGDIILEGLFFGSKEQYEALGLEERFVPKNPGNILVLTDWLGMVGHALEDTILRLVGNTPTWFYAKSLGFTPDTLIPSAGIDEFFEYIENNKAGTSTWFVTLSLEGGAINDVPADATAYGHRDVLFWVQIFMVSPTGPVSSTTYDFADGLYNVLTKAVPESEGHAYLGCPDPKMANAQQKYWRQNLPRLEELKAILDPKDTFHNPQGILPA

SEQ ID NO: 125 - protein, 476 aa, Aspergillus fischerianus
MGNTTSIAAGRDCLLSAVGGNHAHVAFQDQLLYQATAVEPYNLNIPVTPAAVTYPQSADEIAAVVKCAAEYGYKVQARSGGHSFGNYGLGGEDGAIVVEMKHFNQFSMDESTYTATIGPGITLGDLDTGLYNAGHRAMAHGICPTIRTGGHLTMGGLGPTARQWGLALDHVEEVEVVLANSSIVRASDTQNQDIFFAVKGAAASFGIVTEFKVRTEEAPGLAVQYSFTFNLGTAAEKAKLVKDWQAFIAQEDLTWKFYSNMNIFDGQIILEGIYFGSKEEYDALGLEERFPTSEPGTVLVLTDWLGMVGHGLEDVILRLVGNTPTWFYAKSLGFAPRALIPDSAIDDFFSYIHENNPGTVSWFVTLSLEGGAINKVPEDATAYGHRDVLFWVQIFMINPLGPVSQTTYGFADGLYDVLAKAVPESAGHAYLGCPDPRMPNAQQAYWRSNLPRLEELKGELDPKDIFHNPQGVMVVS

SEQ ID NO: 126 - protein, 473 aa, Aspergillus neoniger
MGNTTSIAGRDCLISALGGNSALAAFPNQLLWTADVHEYNLNLPVTPAAITYPETAEQIAGIVKCASDYDYKVQARSGGHSFGNYGLGGTDGAVVVDMKHFNQFSMDDQTYEAVIGPGTTLNDVDIELYNNGKRAMAHGVCPTIKTGGHFTIGGLGPTARQWGLALDHVEEVEVVLANSSIVRASNTQNQDVLFAVKGAAADFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIDNHTAGTPAWFVTLSLEGGAINDVAEDATAYAHRDVLFWVQLFMVNPLGPISETTYEFTDGLYDVLARAVPESVGHAYLGCPDPRMENAPQKYWRTNLPRLQELKEELDPKNTFHHPQGVIPA

SEQ ID NO: 127 - protein, 473 aa, Aspergillus eucalypticola
MGNTTSIAGRDCLISALGGDSALAVFPNQLLWTADVHEYNLNLPVTPAAITYPETAEQIAGIVKCASDYDYKVQARSGGHSFGNYGLGGTDGAVVVDMKHFNQFSMDDQTYEAVIGPGTTLNDVDIELYNNGKRAMAHGVCPTIKTGGHFTIGGLGPTARQWGLALDHVEEVEVVLANSSIVRASNTQNQDVFFAVKGAAADFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIANHTTGTPVWLVTLSLEGGAINDVAEDATAYAHRDVLFWVQLFMVNPLGPISETTYEFTDGLYDVLARAVPESVGHAYLGCPDPRMEDAPQKYWRTNLPRLQELKEELDPKNTFHHPQGVIPA

SEQ ID NO: 128 - protein, 482 aa, Aspergillus ruber
MGNKASTTTIITTAVHKCLLSAVNGNSAQVSVQNDLLYGVTAVHEYNLNFPMTPAAVTFPETSEQVAALVKCAAEYKYKVQARSGGHSFGNHGLGGADGAIVVDMKHFQQFSMDNETHVATIGPGLSLGDIDTLLYNAGGRAMSHGICPEIRAGGHLTIGGLGLTSRQWGMSLDHIEEVEVVLPNSSIVRASETENADLLFAVKGAAASFGVVTEFKVRTQLAPKEAIQYSYSFKLGSAAQRARLFADWQDLALRRDLSRKFTSDFICLQDSVIVKGVFFGSKKEYNALRIEHHLPGSDSSKVLVLDDWLGIVTHVVDDLAVRLGGSMSTYFYAKSLGFTRDTLMPPSTITSLFTYLDKAKKGTITWFVTFSLVGGAINDYPKNATAYPHRDVIYWMQSFAINALGPVLNSTYDFLDGINELVARDLPGCAGHAYLGCPDPRMEGAERAYWGSNLGRLEDMKGVFDPVDVFWNPQGVGVPVA

SEQ ID NO: 129 - protein, 473 aa, Aspergillus kawachii
MGNTTSIAGRDCLISALGGNSALAVFPNQLLWTADVHEYNLNLPVTPAAITYPETAEQIAGIVKCASDYDYKVQARSGGHSFGNYGLGGTDGAVVVDMKHFTQFSMDDQTYEAVIGPGTTLNDVDIELYNNGKRAMAHGVCPTIKTGGHFTIGGLGPTARQWGLALDHVEEVEVVLANSSIVRASNTQNQDVFFAVKGAAADFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIDNHTAGTPAWFVTLSLEGGAINDVAEDATAYAHRDVLFWVQLFMVNPLGPISETTYEFTDGLYDVLARAVPESVGHAYLGCPDPRMENAPQKYWRTNLPRLQELKEELDPKNTFHHPQGVIPA

SEQ ID NO: 130 - protein, 474 aa, Aspergillus oryzae
MGNTTSIAGRDCLVSALGGNAGLVAFQNQPLYQTTAVHEYNLNIPVTPAAITYPETAEQIAAVVKCASQYDYKVQARSGGHSFGNYGLGGTDGAVVVDMKYFNQFSMDDQTYEAVIGPGTTLGDVDVELYNNGKRAMAHGVCPTISTGGHFTMGGLGPTARQWGLALDHVEEVEVVLANSSIVRASNTQNQEVFFAVKGAAASFGIVTEFKVRTQPAPGIAVQYSYTFNLGSSAEKAQFIKDWQSFVSAKNLTRQFYTNMVIFDGDIILEGLFFGSKEQYEALRLEERFVPKNPGNILVLTDWLGMVGHALEDTILRLVGNTPTWFYAKSLGFTPDTLIPSSGIDEFFKYIENNKAGTSTWFVTLSLEGGAINDVPADATAYGHRDVLFWVQIFMVSPTGPVSSTTYDFADGLYNVLTKAVPESEGHAYLGCPDPKMANAQQKYWRQNLPRLEELKETLDPKDTFHNPQGILPA

SEQ ID NO: 131 - protein, 483 aa, Aspergillus flavus
MNPSIPSSSMGNTTSIAGRDCLVSALGGNAGLVAFQNQPLYQTTAVHEYNLNTPVTPAAITYPETAEHIAAVVKCASQYDYKVQARSGGHSFGNYGLGGTDGAVVVDMKYFNQFSMDDQTYEAVIGPGTTLGDVDVELYNNGKRAMAHGVCPTISTGGHFTMGGLGPTARQWGLALDHVEEVEVVLANSSIVRASNTQNQEVFFAVKGAAASFGIVTEFKVRTQPAPGIAVQYSYTFNLGSSAEKAQFIKDWQSFVSAKNLTRQFYTNMVIFDGDIILEGLFFGSKEQYEALGLEERFVPKNPGNILVLTDWLGMVGHALEDTILRLVGNTPTWFYAKSLGFTPDTLIPSSGIDEFFEYIENNKAGTSTWFVTLSLEGGAINDVPADATAYGHRDVLFWVQIFMVSPTGPVSSTTYDFADGLYNVLTKAVPESEGHAYLGCPDPKMANAQQKYWRQNLPRLEELKETLDPKDTFHNPQGILPA

SEQ ID NO: 132 - protein, 616 aa, Aspergillus oryzae
MGNTTSIAGRDCLVSALGGNAGLVAFQNQPLYQTTAVHEYNLNIPVTPAAITYPETAEQIAAVVKCASQYDYKVQARSGGHSFGNYGLGGTDGAVVVDLKYFNQFSMDDQTYEAVIGPGTTLGDVDVELYNNGKRAMAHGVCPTISTGGHFTMGGLGPTARQWGLALDHVEEVEVVLANSSIVRASNTQNQEVFFAVKGAAASFGIVTEFKVRTQPAPGIAVQYSYTFNLGSSAEKAQFIKDWQSFVSAKNLTRQFYTNMVIFDGDIILEGLFFGSKEQYEALRLEERFVPKNPGNILVLTDWLGMVGHALEDTILRLVGNTPTWFYAKSLGFTPDTLIPSSGIDEFFEYIENNKAGTSTWFVTLSLEGGAINDVPADATAYGHRDVLFWVQIFMVSPTGPVSSTTYDFADGLYNVLTKVVPESEGHAYLGCPDPKMANAQQKYWRQNLPRLEELKETLDPKDTFHNPQGVLITEVGSATDFWNLVEAIILISQLHESVGQTYNMVPEMGEQPVREMTKMFRMLEKTIQVSLEGLPYEEWLNRLQVENDDDPLRPLLPMFEEKVYDGRCQWEMYENMPISDTENLRQYLQDVPELATCPFLDQDIFKKFLSSLGLA

SEQ ID NO: 133 - skipped

SEQ ID NO: 134 - protein, 473 aa, Aspergillus awamori
MGNTTSIAGRDCLISALGGNSALAAFPNELLWTADVHEYNLNLPVTPAAITYPETAEQIAGVVKCASDYDYKVQARSGGHSFGNYGLGGADGAVVVDMKHFTQFSMDDETYEAVIGPGTTLNDVDIELYNNGKRAMAHGVCPTIKTGGHFTIGGLGPTARQWGLALDHVEEVEVVLANSSIVRASNTQNQDVFFAVKGAAANFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIANHTAGTPAWFVTLSLEGGAINDIAEDATAYAHRDVLFWVQLFMVNPLGPISDTTYEFTDGLYDVLARAVPESVGHAYLGCPDPRMEDAQQKYWRTNLPRLQELKEELDPKNTFHHPQGVMPA

SEQ ID NO: 135 - protein, 476 aa, Aspergillus lentulus
MGNTTSIAAGRDCLLSAVGGNHAHVAFQDQLLYQATAVEPYNLNIPVTPAAVTYPQSAEEIAAVVQCASEYGYKVQARSGGHSFGNYGLGGEDGAIVVEMKHFNQFSMDESTNIATIGPGITLGDLDTALYNAGYRAMAHGICPTIRTGGHLTMGGLGPTARQWGLALDHVEEVEVVLANSSIVRASDTQNQDIFFAVKGAAASFGIVTEFKVRTEQAPGLAVQYSFTFNLQTPAEKAKLVKDWQAFIAQEDLTWKFYSNMNIFDGQIILEGIYFGSKAEYDALGLEKRFPTSEPGTVLVLTDWLGMVGHGLEDVILRLVGNTPTWFYAKSLGFTPRALIPDSAIDDFFNYIHKNNPGTVSWFVTLSLEGGAINKVPEDATAYGHRDVLFWVQIFMINPLGPVSQTTYGFADGLYDVLAKAVPESAGHAYLGCPDPRMPNAQQAYWRSNLPRLEELKGELDPKDVFHNPQGVMVVS

SEQ ID NO: 136 - protein, 473 aa, Aspergillus turcosus
MGNTTSIAAGRDCLISAVGAANVAFQDQLLYQATAVQPYNLNIPVTPAAVTYPQSADEIAAVVKCASEYGYKVQARSGGHSFGNYGLGGQDGAIVIEMKHFSQFSMDESTFIATIGPGITLGDLDTDLYNAGHRAMAHGICPTIRTGGHLTVGGLGPTARQWGLALDHVEEVEVVLANSSIVRASDTQNQDLFFAIKGAAASFGIVTEFKVRTEQAPGMAVQYSYTFHLGTSAEKAKFVKDWQAFIAQENLTWKFYTNLVIFDDQIILEGIYFGTKEEYDSLGLEQRFPPTDAGTVLILTDWLAMIGHGLEDTILKLVGDTPTWFYAKSLGFTPRALIPDSAIDEFFDYIHENNPGTLAWFVTLSLEGGAINAVPEDATAYGHRDVLFWFQLFVINPLGPISQTTYGFADGLYDVLAQAVPESVSHAYMGCPDPRLPNAQYAYWRSNLPKLEELKGILDPEDIFHNPQGVVPS

SEQ ID NO: 137 - protein, 496 aa, Aspergillus welwitschiae
MLSTMAFSFVLRILSPLFLILQLSTAASTSTLRQCLLTAVQNDPTLVAVDGDLLYQTLAVQVYNLNWPVTPAAVAFPKSTQQVASIVNCAASLGYKVQAKSGGHSYGNYGLGGTNGAISINLKNMKSFSMNYTNYQATVGAGMLNGELDEYLHNAGGRAVAHGTSPQIGVGGHATIGGLGPSARQYGMELDHVLEAEVVLANGTVVRASSTQNSDLLFAIKGAGASFGVVTEFVFRTEPEPGSAVQYTFTFGLGSTSARADLFKKWQSFISQPDLTRKFASICTLLDHVLVISGTFFGTKEEYDALGLEDQFPGHTNSTVIVFTDWLGLVAQWAEQSILDLTGDIPADFYARCLSFTEKTLIPSNGVDQLFEYLDSADTGALLWFVIFDLEGGAINDVPMDATGYAHRDTLFWLQSYAITLGSVSETTYDFLDSVNEIIRNNTPGLGNGVYPGYVDPRLENAREAYWGSNLPRLMQIKSLYDPTDLFHNPQGVLPA

SEQ ID NO: 138 - protein, 473 aa, Aspergillus niger; identical to SEQ ID NO: 27

SEQ ID NO: 139 - protein, 492 aa, Fusarium floridanum
MRSQLLHGLIGLVALVSPSFAVPTKREAVTSCLTNAKVPIDAKGSQTWTQDGTAYNLRLQFEPIAIAVPTTVAQISAAVACGSKHGVSVSGKSGGHSYTSLGLGGEDGHLVIELDRLYSVKLAKDGTAKIQPGARLGHVATELYNQGKRALSHGTCTGVGLGGHALHGGYGMVSRKHGLTLDSIIGATVVLYDGKVVHCSKTERSDLFWAIRGAGASFGIVAELEFNTFPAPEQMTYFDIGLNWDQNTAAQGLWEFQEFGKTMPSEITMQIAIRKDGYSIDGAYIGDEAGLRKALQPLLSKLNVQVSASTVSWMGLVTHFAGTAEINPTSASYDAHDTFYATSLTTRELSLEQFKSFVNSISTTGKSSSHSWWVQMDIQGGKYSAVAKPKPTDMAYVHRDALLLFQFYDSVPQGQTYPSDGFSLLTTLRQSISKSLRAGTWGMYANYPDSQLKADRAAEMYWGSNLPRLQKIKAAYDPKNIFRNPQSVKPKA

SEQ ID NO: 140 - protein, 475 aa, Paecilomyces variotii
MGNTTSTSAGQCLLSAVGGNPALVAFQNAPLYQAVDVRPYNLDVPVTPVAVTTPETVDQVASIVKCAADAGYKVQPKSGGHSYGNYGLGGVDGEVVVDLKNFQQFSMNNETWRATIGAGTLLGDVTTRLYNAGGRAMAHGTCPQVGIGGHATIGGLGPTSRLWGAALDHIEEVQVVLANSSIVRASQTENPDLLFALKGAGASFGIITEFTVRTEPAPGEAVQYSYTFNFGDNASKAKTFKDWQAFVSTPNLNRKFAATMTVLEDAIVASGTFFGTKEEFDAFELESHFPENQGSNVTVVQDWLGLVADWAEDAALEGGGGVPSAFYAKSLNFSPDTLIPNDTIDDMFDYFSTTEKDALLWFAIFDLSGGAVSDVPVHSTSYTHRDTLFWLQSYAISVGPVSNTTIQFLDGLSNLLTSSQPEVHFGAYPGYVDPKLPDGQLAYWGSNLPKLEQIKAEVDPNDVFHNPQSVKPAKQ

SEQ ID NO: 141 - protein, 704 aa, Verticillium longisporum
MAQPPSSAFATCLNDVCGGRSGCVGYPSDILYQINWVDRYNLDINLEPAAVTKPEITEDVAAFIKCASENNVKVQARSGGHSYANHGLGGEDGALVIDLENFQHFSMNWDNWQATIGAGHKLHDVTEKLHDNGGRAISHGTCPGVGLGGHATIGGLGPSSRMWGSCLDHVVEVEVVTADGKIQRASEDENSDLFFALKGAGASFGIITEFVMRTNEEPGDVVEYTFSLTFSRHRDLSPVFEAWQNLISDPDLDRRFGSEFVMHELGAIITGTFFGTEEEFEATGIPDRIPTGKKSIVVNDWLGSVAQQAQDAALWLSDLSTAFTAKSLAFTKDQLLSSESIMDLMDYIDDANRGTLIWFLIFDVTGGRINDVPMNATAYRHRDKVMFCQGYGIGIPTLNGRTREFIEGINSLIRSSVPTNLSTYAGYVDASLESPQDSYWGPNLDALGQVKEDWDPSDLFSNPQSVRPGQKSVVDYFDNRASSNGSEDSSGGSNGGTRDEQGGCWSWRRSGPAFAVFVALFVGFPTPQTSWVQKQNLRDPALDLTDAESPSRTPVVNPNTLTTDTMAKLSRGAPGGKLKMTLGLPVGAVMNCADNSGARNLYIISVKGIGARLNRLPAGGVGDMVMATVKKGKPELRKKVHPAVIVRQSKPWKRFDGVFLYFEDNAGVIVNPKGEMKGSAITGPVGKEAAELWPRIASNSGVVM

SEQ ID NO: 142 - protein, 486 aa, Penicillium antarcticum
MGNSASVAGRACFVAAVGHDPNLVTFRGDLLYEFRIQPSYNLAIPVHPTVVTYPKTTAQVAEIVSCAAAQNYKMQAYSGGHSYGNYGLGGEDGHVVVDLKNFQDFTMDPDTHVATIGAGTSLGDLQDRLWHAGGRAMAHGSCPQVGVGGHFTIGGLGMMSRQWGMSLDHVVEAQVVLANSSVVTASDTQNQDIFWAIKGAAASFGIVTKFKVRTHGVPKAAIQYQYTFSQGDVLDKVKLFMAWQNIVAKPNLTRNFSTELTIFQDGIMIMGSFFGTRDEFHKFELENDLPLQGLGNVAYITNWLSLVAHTAEDYLLRLTGNVLTSFYAKSLSFTADELFNEQGLVTLFTYLDAAPKGTPTWWVIFDLEGGATNDVPVNATSYAHRDAIMWMQSYAVAGFEPPGFIIKRFLNRLHGVVIGNRAPGAVRSYPGYVDPYLRNAQETYWGPNLARLQDIKTAVDPDDVFHNPQSVKVNSLSPPDPGSHDV

SEQ ID NO: 143 - protein, 473 aa, Unknown
  Monosporascus sp. mg162
MGQTPSSPLADCLNAVCNGRDNCVAFPSAPLYQISWVDRYNLDIEVEPIAVTRPETAEDVSGFVKCAAAHNIKVQAKSGGHSYANYGLGGEDGELVVDLRNFQDFSIDTNTWQATFGAGHKLDDVTEKLHKNGKRAISHGTCPGVGIGGHATIGGLGPESRMWGSCLDHVIGVEVVTADGSIVHASDTENSDLFFALKGAGASFGIVTSFVVKTRPEPGSVVQYSYSVTFAKHADLSPVFRQWQELVMDPGLDRRFGTEFTMHELGVIISGTFYGTDEEFQATGIPDRIPKGKISVVFDDWMAVIAKHAEEAALSLSSISSAFTARSLAFRREDKISPETITNLMNYIDSADRGTLVWFLIFDATGGAISDVPTNATAYSHRDKVMYCQGYGVGIPTLNQQTKDFLSGIINTIQSGAGNTLTTYPGYVDPALTNPQESYWGPNIDTLRAIKSQWDPNDIFHNPQSVRPAAVAA

SEQ ID NO: 144 - protein, 506 aa, Amanita muscaria
MLKTIAAVVFICSQAFLVRADLKSELTALGVGAVFPGDSVYTSDAKPYNLRFDFKPAAITFPNTPADVSQIVQIAGKYAHKVAPRGGGHSYISNGVGGMDNSIIADMSHFKSIVVHTNNDTATIETGNRLGDIALALFQYGRGMPHGACPYVGIGGHANFGGFGFISRSWGLTLDVVEAIDLVLANGTITTVSATQNPDLYWAMRGSGSSFGITTAIHVRTFSAPASGIIALDTWYLNLEQAVRALSSFQDFAHNTVTLPSYFGGEFVVNAGPSPGLLSITFFSGFWGPPNQYNSTLAPWKNSMPFPPNTTSYSQGNYIESLSARFGGAPLDTSLGPDNTDTFYVKSLIVPQVTISDEGAQVGISDKAWRALFQYLINEQPNLPVDWFIEVELWGGQNSAINAVPQASTAFAYRDLLWTLQMYSYTPNHQPPYPDAGFAFNDGMANSIIHNMPNGWNYGAYTNYVDNRLDDWQRLYYANHYPALQALKSRYDPSDTFSFPTSIELL

SEQ ID NO: 145 - protein, 988 aa, Aspergillus niger
MTTNGIQPGHVGNLTQDQEAKLQQLWSIVLTLLDVKSLQGGDTSAQTQPDQRPSTSLSRADTVVSAHGQTAFTEDLSQVLRENGMSNPDIKSVRESLSNTSIDELRSGLLYTAKHDSPDVLLLRFLRARKWDVGKAFGMMLRALVWRKDQHVDDKVIANPELAALVTSQNTVDTHAAKECKDFLDQMRMGKCYMHGTDRDGRPVLVVRVRFHQPSKQSEAVINRFILHTIETARLLLAPPQETVTIIFDMTGFGLSNMEYAPVKFIIECFQENYPESLGYMLIHNAPWVFSGIWKIIKGWMDPVIVSKVNFTNKVSDLEKFIAPEQIVKELKGKEDWTYEYVEPVAGENELMADTETRDRIYAERLKIGEELLLRTSEWVSTSQRKDAAATTTAREQRSETIESLRQNYWQLDPYVRGRTFLDRTGVVKPGGKIDFYPSPDLEPSTAKMLEVEHFERTQFDPYLFLLPHGARIAVRHCSVTALPTYLKAHPRGMLSTMAFSFLRVLSSLLLVLQLSTAASTSTLRQCLLTAVQNDPTLVAVDGDLLFQTLAVQVYNLNWPVTPAAVAFPKSAQQVSSIVNCAASLGYKVQAKSGGHSYGNYGLGGTNGAISINLKNMKSFSMNYTNYQATVGAGMLNGELDDYLHNAGGRAIAHGTSPQIGVGGHATIGGLGPAARQYGMELDHVLEAEVVLANGTVVRASSTQNSDLLFAIKGAGASFGVVTEFVFRTEPEPGSAVQYSFTFGLGSTSSRADLFKKWQSFISQPDLTRKFASICTILDHVLVISGTFFGTKAEYDALGLEDQFPGHTNSTVIVFTDWLGLVAQWAEQSILDLTGGIPADFYSRCLSFTEKTPIPSTGVDQLFEYLDSADTGALLWFVIFDLEGGAINDVPMDATGYAHRDTLFWLQSYAITLGSVSQTTYDFLDRVNEIIRNNTPGLGNGVYPGYVDPRLQNAREAYWGSNLPRLMQIKSLYDPSDLFHNPQGVLPA

SEQ ID NO: 146 - protein, 479 aa, Venturia effusa
MGQGQSSAGGLQDCLTSAVGSGNLAVPSKPFYQQTDVKPYNLDIHVHPVAVTYPQTNEDVAAIVRCAKEHEAKVQPRSGGHSYGNFATGNGNDNMIVVDLKHFKQFSMDDNTWIATLGSGHLLGDVTKKLLANGGRAMAHGTCPQVGIGGHATIGGLGPMSRMWGSSLDHVQEITVVLANSSIITASPTQNKDVFWAMKGAGASFGIITEFKVITHPAPGEAVKYSFGFSGGSHRDQAKRFKKWQSMIADPGLSRKLASQVVLSEIGMIISGTFFGTQAEYNQLNLTSVFPEMSSHKIIVFNDWAGLVGHWAEDVGLQLGGGISSPFYSKSLAFTPNDLIPAEGIDRFFEYLDEVDKGTLIWFGIFDLEGGATNDIPADATAYGHRDALFYFQSYGVNLGLKVKDETRDFINGMNSVLEGSLSNHKLGAYAGYVDPALSLEAAQVGYWGDNLPRLRQIKRAVDPDDVFHNLQSVRPAAS

SEQ ID NO: 147 - protein, 472 aa, Lecanicillium lecanii
MGNKPSTPLAHCLRDVCAGRGNCVAFPNEYLYQANWVKPYNLDVPVKPIAVFRPDNAADVAAAVKCAGQSSVHVQAKSGGHSYANFGLGGGDGGLMIDLQHLNKFSMNNETWQATFGSGFLLGDLDKQLHANGNRAMAHGTCPGVGIGGHATIGGIGPSSRMWGTALDHVLEVEVVTADGKIQRASKTQNSDLFWGLQGAGASFGIITEFVVRTEPEPGSVVEYAYSLNFGKQADMAPVYKKWQDLVGDPNLDRRFTSLFIAEPLGVLITGTFYGTLDEYKASGIPDKLPASGASITVMDWLGSLAHIAEKTGLYLSNVSTKFVSRSLALREEDLLSEQSIDDLFKYMGSADADTPLWFVIFDNEGGAIADVPDNSTAYPHRDKIILYQSYSVGLLGVSDKMINFVDGIQDLVQKGAPNAHTTYAGYINANLDRNAAQKFYWGDKLPQLQQLKKKFDPTSLFSNPQSIDPAD

SEQ ID NO: 148 - protein, 474 aa, Penicillium chrysogenum
MGNTTSIAAGRDCLVSAVGPAHVTFQDALLYQTTAVDPYNLNIPVTPAAVTYPQSAEEIAAVVKCASDYDYKVQARSGGHSFGNYGLGGQNGAIVVDMKHFSQFSMDESTFVATIGPGTTLGDLDTELYNAGGRAMAHGICPTIRTGGHLTVGGLGPTARQWGLALDHIEEVEVVLANSSIVRASNTQNQDILFAVKGAAASFGIVTEFKVRTQEAPGLAVQFSFTFNLGSPAQKAKLVKDWQAFIAQENLSWKFYSNLVIFDGQIILEGIFFGSKEEYDELDLEKRFPTSEPGTVLVLTDWLGMIGHALEDTILKLVGDTPTWFYAKSLGFTPDTLIPDSAIDDFFDYIHKTNAGTLAWFVTLSLEGGAINSVSEDATAYGHRDVLFWFQVFVVNPLGPISQTTYDFTNGLYDVLAQAVPESAGHAYLGCPDPKMPDAQRAYWRSNLPRLEDLKGDLDPKDTFHNPQGVQVGP

SEQ ID NO: 149 - protein, 496 aa, Aspergillus lacticoffeatus
MLSTMAFSFVLRILSPLFLILQLSTAASTSTLRQCLLTAVQNDPTLVAVDGDLLYQTLAVQVYNLNWPVTPAAVAFPKSTQQVASIVNCAASLGYKVQAKSGGHSYGNYGLGGTNGAISINLKNMKSFSMNYTNYQATVGAGMLNGELDEYLHNAGGRAVAHGTSPQIGVGGHATIGGLGPSARQYGMELDHVLEAEVVLANGTVVRASSTQNSDLLFAIKGAGASFGVVTEFVFRTEPEPGSAVQYTFTFGLGSTSARADLFKKWQSFISQPDLTRKFASICTLLDHVLVISGTFFGTKEEYDALGLEDQFPGHTNSTVIVFTDWLGLVAQWAEQSILDLTGGIPADFYARCLSFTEKTLIPSNGVDQLFEYLDSADTGALLWFVIFDLEGGAINDVPMDATGYAHRDTLFWLQSYAITLGSVSETTYDFLDNVNEIIRNNTPGLGNGVYPGYVDPRLQNAREAYWGSNLPRLMQIKSLYDPTDLFHNPQGVLPA

SEQ ID NO: 150 - protein, 468 aa, Aspergillus ochraceoroseus
MAPSISFSLLQISLLAYSGLVSGDFSLRQCLESAVSRVAFEGDPFYQLLSVRPYNLDISIVPAAVAFPADTNEVAAVVRCAAQNGYQVQAKSGGHSYANHGLGGTNGAVVVNLENLQHFSMNTTTWEATIGAGTLLGDVTKRLSDAGGRAMAHGTCPQVGSGGHFTIGGLGPSSRQFGAALDHIIEAEVVLANSSIIRASETENPDVFFAVRGAASGFGIVTEFKVRTEPEPGQAVRYSYSFSFSDTATRADLFKKWQAYVTQPDLPRELASTLTILEHGMFITGTFFGSKEEYNALKIETEFPGFAKGGTLVLDDWLGLVSNWAEDLLLSEEEIEQMFEYIDNVDKGTLLWFAIFDLQGGAVGDVPVDATAYAHRDTLIWLQSYAINLFGRISETTVEFLERLNELTLTSTAKTVPYAAYPGYVDPRLTDAQAAYWGSNLARLNRIKAEIDPNNVFHNPQSVRPASG

SEQ ID NO: 151 - protein, 475 aa, Aspergillus cristatus
MGNTTSIAAGRDCLVNAVGGNQALVAFQDQLLYQSTAVEAYNLNIPVTPAAVTFPESSEQIAAVVKCASEHDYKVQARSGGHSFGNYGLGGTNGAIVVDMKKFDQFSMDESSYIATIGPGTTLGDVDTELYNAGGRAMAHGICPTIRTGGHLTMGGLGPTARQWGLALDHIEEVEVVLANSSIVRASHTQNQDILFAVKGASASFGIVTEFKVRTEPAPGLAVQYSYTFNLGSAASKAKLVKDWQEFIAQDNLTWKFYSNMVIIDGDIILEGIFFGSKEEFDALELENRFPPKNPGNILVLTDWLGMISHSLEDIILRVAGGVPTYFYAKSLGFTPQALIPSSAIDDLFDYIEKTNPGTLAWFITLSLEGGAINNVPADATAYGHRDVLHWVQIFAANPLGPISETTYDFTDGLYNILAKAVPESAEHAYLGCPDPRMKDAQKAYWRDNLPRLEELKAELDPKDTFHNPQGVAVA

SEQ ID NO: 152 - protein, 488 aa, Penicillium roqueforti
MLLKLFFLAVAASVALAASSEALKQCLENVFTDRAGFAFAGDLFYDRIVNRYNLNIPVTPSALAFPTSSQQVADIVKCAADNGYPVQARSGGHSYGNYGLGGADGAVAIDLKHLQQFSMDKTTWQATIGAGSLLSDVTQRLSHAGGRAMSHGICPQVGSGGHFTIGGLGPTSRQFGAALDHVLEVEVVLANSSIVRASDTENKDLFWAIKGAASGYGIVTEFKVRTEPEPGTAVQYAYSMEFGNPTKQATLFKSWQAFVSDPKLTRKMASTLTMLENSMAISGTFFGTKEEYDKLNLTNKFPGANGDALVFEDWLGLVAHWAEDLILGLAAGIPTNFYAKSTSWTPQTLITPETVDKMFDYIATVNKGTLGWFLLFDLQGGYTNDIPTNATSYAHRDVLIWLQSYTVNFLGPISQAQIDFLDGLNKIVTNNKLPYTAYPGYVDPLMPNAPEAYWGTNLPRLQQIKELVDPNDVFRNPQSPSPANKEPL

SEQ ID NO: 153 - protein, 472 aa, Fusarium sporotrichioides
MGNGNSTPFRDCLDSICANRSTCVTYPGDPLFSCWSRPFNLEFPVVPAAIIRPETTTEVAETVKCAKKYGYKVQAKSGGHSYGNHGLGGVGGAVSIDMVNLRDFSMNNKTWYASFGSGMNLGELDEHLHANGRRAIAHGTCPGVGTGGHLTVGGLGPISRQWGSALDHLLEIEVITADGTVQRASYTKNSGLFWALRGAGASFGIVTKFMVKTHPEPGRVVQYSYNIALASHAETAELYREWQALVGDPNMDRRFSSLFVVQPLGALITGTFFGTKSQYQATGIPDRLPGADKGAVWLTDWAGLLLHEAEAAGCALGSIPTAFYGKSLSLSEQDLLSDSAITDLFKYLEDNRSGLAPVTILFNTEGGAMMDTPANATAYPHRNSIIMYQSYGIGVGKVSAATRKLLDGVHERIQRSAPGALSTYAGYIDAWADRKAAQKLYWADNLPRLRELKKVWDPADVFSNPQSVEPAD

SEQ ID NO: 154 - protein, 371 aa, Beauveria bassiana
MDSNTWEATFGSGFLLGELDKHLHANGNRAMAHGTCPGVGMGGHATIGGIGPSSRLWGTTLDHVLQVEVVTADGKIQRASKTQNPDLFWALQGAGASFGIITEFVVRTEPEPGSVVEYTYSVSLGKQSDMAPLYKQWQALVGDPSLDRRFTSLFIAEPLGVLITGTFYGTMYEWHASGIPDKLPRGPISVTVMDSLGSLAHIAEKTGLYLTNVPTSFASRSLALRQQDLLSEQSIDDLFEYMGSANADTPLWFVIFDNEGGAIADVPDNSTAYPHRDKVIVYQSYSVGLLGVTDKMIKFLDGVQDIVQRGAPNAHTTYAGYINPQLDRKAAQQFYWGDKLPRLQQIKKQYDPNNVFCNPQSIYPAEDMSDG

SEQ ID NO: 155 - protein, 473 aa, Aspergillus vadensis
MGNTTSIAGRDCLVSALGGNSALAAFPNQLLWTADVHEYNLNLPVTPAAITYPETAEQIAGIVKCASDYDYKVQARSGGHSFGNYGLGGTDGAVVVDMKHFNQFSMNDQTYEAVIGPGTTLNDVDIELYNNGKRAMAHGVCPTIKTGGHFTIGGLGPTARQWGLALDHVEEVEVVLANSSIVRASNTQNQDVFFAVKGAAADFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIANHTAGTPAWFVTLSLEGGAINDVAEDATAYAHRDVLFWVQLFMVNPLGPISETTYEFTDGLYDVLARAVPESVGHAYLGCPDPRMENAPQKYWRTNLPRLQELKEELDPKNTFHHPQGVIPA

SEQ ID NO: 156 - protein, 476 aa, Penicillium arizonense
MGNTTSIAAGRDCLVSAVGGVAAHVAFQDSLLYQATAVELYNLNIPVTPAAVTYPQSTDEIAAVVKCASDYDYKVQARSGGHSFGNYGLGGQNGAIVIDMKHFSQFSLDKSTFIATFGPGTTLGNLDTELYHAGNRAMAHGICPTIRTGGHLTMGGLGPAARQWGLALDHVEEVEVVLANSSVVRASDTQNQDVFFAVKGAAASFGIVTEFKVRTEEAPGLAVQYSFPFNLGTPAEKAKLVKDWQAFIAQENLSWKFYSNMVIFDGQIILEGIFFGSKKEYDELDLENKFPTSEPGTVLVLTDWLGMIGHGLEDTILRLVGNSPTWFYAKSLGFTPSTLISDSAIDGLFDYIHKTNPGTLAWFVTLSLEGGAINTVSEDATAYGHRDVLFWVQIFVANPLGPISQTTYDFADGLYNVLAQAVPDSAGHAYLGCPDPKLPDAQRAYWRSNLPRLEELKRDLDPKDIFYNPQGVQIVS

SEQ ID NO: 157 - protein, 476 aa, Aspergillus fumigatus; identical to SEQ ID NO: 109

SEQ ID NO: 158 - protein, 473 aa, Purpureocillium lilacinum
MGNGQSTPLQQCLNTVCNGRLGCVAFPSDALYQAAWVKPYNLDVPVTPIAVFKPSSTEDVAGAIKCAVASNVHVQAKSGGHSYANFGLGGQDGELMIDLANLQDFHMDKTSWQATFGAGYRLGDLDKKLQANGNRAIAHGTCPGVGIGGHATIGGLGPMSRMWGSALDHVLSVQVVTADGSIKNASESENSDLFWALRGAGASFGVITKFTVKTHPAPGSVVQYTYKISLGSQAQMAPVYAAWQALAGDPKLDRRFSTLFIAEPLGALITGTFYGTKAEYEATGIAARLPSGGTLDLKLLDWLGSLAHIAEVVGLTLGDIPTSFYGKSLALREEDMLDRTSIDGLFRYMGDADAGTLLWFVIFNSEGGAMADTPAGATAYPHRDKLIMYQSYVIGIPTLTKATRDFADGVHDRVRMGAPAANSTYAGYIDRTLSREAAQEFYWGAQLPRLREVKKAWDPKDVFHNPQSVDPAE

SEQ ID NO: 159 - protein, 473 aa, Aspergillus turcosus
MGNTTSIAAGRDCLISAVGAANVAFQDQLLYQATAVQPYNLNIPVTPAAVTYPQSADEIAAVVKCASEYGYKVQARSGGHSFGNYGLGGQDGAIVIEMKHFSQFSMDESTFIATIGPGITLGDLDTDLYNAGHRAMAHGICPTIRTGGHLTVGGLGPTARQWGLALDHVEEVEVVLANSSIVRASDTQNQDLFFAIKGAAASFGIVTEFKVRTEQAPGMAVQYSYTFHLGTSAEKAKFVKDWQAFIAQENLTWKFYTNLVIFDDQIILEGIYFGTKEEYDSLGLEQRFPPTDAGTVLILTDWLAMIGHGLEDTILKLVGDTPTWFYAKSLGFTPRALIPDSAIDEFFDYIHENNPGTLAWFVTLSLEGGAINAVPEDATAYGHRDVLFWFQLFVINPLGPISQTTYGFADGLYDVLAQAVPESASHAYMGCPDPRMPNAQRAYWRSNLPKLEELKGYLDPEDIFHNPQGVVPS

SEQ ID NO: 160 - protein, 476 aa, Penicillium solitum
MGNTTSITAGRDCLTSAVGGVAAHVAFQDALLYQTPAVDPYNLNIPVTPAAVTYPQSADEVAAVVKCASDYNYKVQARSGGHSFGNFGLGGQNGAIVVDMKHFSQFSMDESTFVATIGPGTTLGNLDTEIYNAGKRAMSHGICPSIRTGGHLTVGGLGPTARQWGLALDHVEEVEVVLANSSIIRASDTQNQDVLFAIKGAAASFGIVTEFKVRTEEAPGLAVQYSFTFNLGTPAEKAKLVKDWQAYIAQENLTWKFYSNLIIFDGQIILEGIFFGSKEEYDQLNLDKKFPTSEPGTVLVLTDWLGMIGHGLEDTILRLVGDSPTWFYAKSLGFTPSTLISGSAIDGLFDYIHKTNAGTLAWFVTLSLEGGAINAVPKDATAYGHRDVLFWVQIFVANPLGPISQTTYDFTDGLYDILAQAVPESAGHAYLGCPDPKMPDAQRAYWRSNLPRLEELKGDLDPKDIFHNPQGVQVAS

SEQ ID NO: 161 - protein, 483 aa, Aspergillus flavus
MNPSIPSSSMGNTTSIAGRDCLVSALGGNAGLVAFQNQPLYQTTAVHEYNLNIPVTPAAITYPETAEQIAAVVKCASQYDYKVQARSGGHSFGNYGLGGTDGAVVVDMKYFNQFSMDDQTYEAVIGPGTTLGDVDVELYNNGKRAMAHGVCPTISTGGHFTMGGLGPTARQWGLALDHVEEVEVVLANSSIVRASNTQNQEVFFAVKGAAASFGIVTEFKVRTQPAPGIAVQYSYTFNLGSSAEKAQFIKDWQSFVSAKNLTRQFYTNMVIFDGDIILEGLFFGSKEQYEALGLEERFVPKNPGNILVLTDWLGMVGHALEDTILRLVGNTPTWFYAKSLGFTPDTLIPSSGIDEFFEYIENNKAGTSTWFVTLSLEGGAINDVPADATAYGHRDVLFWVQIFMVSPTGPVSSTTYDFADGLYNVLTKAVPESEGHAYLGCPDPKMANAQQKYWRQNLPRLEELKETLDPKDTFHNPQGILPA

SEQ ID NO: 162 - protein, 473 aa, Aspergillus lacticoffeatus
MGNTTSIAGRDCLISALGGNSALAVFPNELLWTADVHEYNLNLPVTPAAITYPETAAQIAGVVKCASDYDYKVQARSGGHSFGNYGLGGADGAVVVDMKHFTQFSMDDETYEAVIGPGTTLNDVDIELYNNGKRAMAHGVCPTIKTGGHFTIGGLGPTARQWGLALDHVEEVEVVLANSSIVRASNTQNQDVFFAVKGAAANFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIANHTAGTPAWFVTLSLEGGAINDVAEDATAYAHRDVLFWVQLFMVNPLGPISDTTYEFTDGLYDVLARAVPESVGHAYLGCPDPRMEDAQQKYWRTNLPRLQELKEELDPKNTFHHPQGVMPA

SEQ ID NO: 163 - protein, 476 aa, Aspergillus novofumigatus
MGNTTSIAAGRDCLLSAVGGNHAHVAFQDQLLYQVTAVEPYNLNIPVTPAAVTYPQSADEIAAVVKCASEYGYKVQARSGGHSFGNYGLGGEDGAIVVEMKHFNQFSMDESTYTATIGPGITLGDLDTALYNAGHRAMAHGICPTIRTGGHLTMGGLGPTARQWGLALDHVEEVEVVLANSSIVRASNTQNQDILFAIKGAAASFGIVTEFKVRTEAAPGVAVQYSFTFNLGTPAEKAKLVKDWQAFIAQEDLTWKFYSNMNIFDGQIILEGIYFGSKEEYDALGLEKRFPSSEAGTVLVLTDWLGMVGHGLEDVILRLVGNTPTWFYAKSLGFTPRALIPDSAIDEFLNYIHENTPGTVSWFVTLSLEGGAINKVPGDATAYGHRDVLFWVQIFMINPLGPVSQTTYGFADGLYDVLAKAVPNSAGHAYLGCPDPRMPNAQQAYWRSNLPRLEELKGELDPKDIFHNPQGVMVVS

SEQ ID NO: 164 - protein, 473 aa, Aspergillus welwitschiae
MGNTTSIAGRDCLISALGGNSALAAFPNELLWTADVHEYNLNLPVTPAAITYPETAEQIAGIVKCASDYDYKVQARSGGHSFGNYGLGGADGAVVVDMKHFTQFSMDDETYEAVIGPGTTLNDVDIELYNNGKRAMAHGVCPTIKTGGHFTIGGLGPTARQWGLALDHVEEVEVVLANSSIVRASNTQNQDVFFAVKGAAANFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIANHTAGTPAWFVTLSLEGGAINDIAEDATAYAHRDVLFWVQLFMVNPLGPISDTTYEFTDGLYDVLARAVPESVGHAYLGCPDPRMEDAQQKYWRTNLPRLQELKEELDPKNTFHHPQGVMPA

SEQ ID NO: 165 - protein, 473 aa, Aspergillus niger; identical to SEQ ID NO: 27

SEQ ID NO: 166 - protein, 476 aa, Aspergillus terreus
MGNTTSIAAGRDCLISAVGGNAAHVAFQDQLLYQATAVDVYNLNIPVTPAAVTYPQSADEVAAVVKCASEYDYKVQARSGGHSFGNYGLGGQNGAIVVDMKHFSQFSMDESTYTATIGPGITLGDLDTELYNAGHRAMAHGICPTIRTGGHLTIGGLGPTARQWGLALDHVEEVEVVLANSSIVRASETQNQDVLFAVKGAAASFGIVTEFKVRTEQAPGLAVQYSYTFNLGTPAEKAKLLKDWQAFIAQEDLTWKFYSNMVIFDGQIILEGIFFGSKEEYDALDLEKRFPTSEPGTLLVLTDWLGMVGHSLEDVILRLVGNTPTWFYAKSLGFTPRTLIPDSAIDRFFDYIHETNAGTLAWFVTLSLEGGAINAVPEDATAYGHRDVLFWVQIFMVNPLGPISQTIYDFADGLYDVLAQAVPESAEHAYLGCPDPKMPDAQRAYWRGNLPRLEELKGEFDPKDTFHNPQGVSVAV

SEQ ID NO: 167 - protein, 476 aa, Aspergillus udagawae
MGNTTSIAAGRDCLISAVGGNAAHVAFQDQLLYQATAVEPYNLNIPITPAAITYPQSADEIAAVVKCASEYGYKVQARSGGHSFGNYGLGGEDGAIVVEMKHFSQFSMDESTYIATIGPGITLGDLDTELYNVGHRAMAHGICPTIRTGGHLTVGGLGPTARQWGLALDHVEEVEVVLANSSIVRASDTQNQDIFFAIKGAAASFGIVTEFKVRTEQAPGLAVQYSYTFNLGTPAEKAKLVKDWQAFIAQENLSWKFYSNMVVFDGQIILEGLYFGSKEEYDALGLEQRFPPSEAGNVLVLTDWLGMVGHELEDTILRLVGNTPTWFYAKSLGFTPRALIPDSAIDDLFNYIHENNPGTLAWFVTLSLEGGAINTVPEHATAYGHRDVLFWVQIFVINPLGPVSQTTYGFADGMYDVLAQAVPESAGHAYLGCPDPRMPNAQQAYWRSNLPRLEELKGDLDPKGIFHNPQGVMVVS

SEQ ID NO: 168 - protein, 476 aa, Aspergillus fumigatus; identical to SEQ ID NO: 109

SEQ ID NO: 169 - protein, 474 aa, Aspergillus bombycis
MGNTTSIAGRDCLVSALGGNAGLVAFQDQLLYQTTAVHEYNLNIPVTPAAVTYPETAEQIAAVVKCASEYDYKVQARSGGHSFGNYGLGGADGAVVVDMKHFSQFSMDDQTYEAVIGPGTTLGDVDTELYNNGKRAMAHGICPTISTGGHFTMGGLGPTARQWGLALDHVEEVEVVLANSSIVRASNTQNQEVFFAVKGAAASFGIVTEFKVRTQPAPGLAVQYSYTFNIGSSAEKAQFVKDWQSFISAKNLTRQFYTNMVIFDGDIILEGLFFGSQEQYEALGLEDRFVPKNPGNILVLTDWLGMVGHALEDTILRLVGNTPTWFYAKSLGFTPDTLIPASGIDEFFDYIENHKAGTLTWFVTLSLEGGAINDVPEDATAYGHRDVLFWVQIFMASPTGPVSSTTYDFADGLYNVLTKAVPESEGHAYLGCPDPKMADAQQKYWRQNLPRLEELKATLDPKDTFHNPQGILPA

SEQ ID NO: 170 - protein, 694 aa, Purpureocillium lilacinum
MWLSTMNGSASRRSDPVSRKIVCDGHASAHEVRTDNEAARDVPSRTAVNKERKQGSGPPGAMQRGFHAAHKPNEMVPQDGPLGRTAQLFRLAPACQSEPTRAPGQPSDLRLRQIPLATEQAARTLARMRPARFTFPYGRAAEDDCYLKKEDEGHDQSHPTSVLVGVPPFTRRCAAAETFKDTRARAPGTQPTDTTSTGASPSWTLSPLLSLSATDDSVPSKMGNGQSTPLQQCLNTVCNGRLGCVAFPSDALYQAAWVKPYNLDVPVTPIAVFKPSSTEDVAGAIKCAVASNVHVQAKSGGHSYANFGLGGQDGELMIDLANLQDFHMDKTSWQATFGAGYRLGDLDKKLQANGNRAIAHGTCPGVGIGGHATIGGLGPMSRMWGSALDHVLSVQVVTADGSIKNASESENSDLFWALRGAGASFGVITKFTVKTHPAPGSVVQYTYKISLGSQAQMAPVYAAWQALAGDPKLDRRFSTLFIAEPLGALITGTFYGTKAEYEATGIAARLPSGGTLDLKLLDWLGSLAHIAEVVGLTLGDIPTSFYGKSLALREEDMLDRTSIDGLFRYMGDADAGTLLWFVIFNSEGGAMADTPAGATAYPHRDKLIMYQSYVIGIPTLTKATRDFADGVHDRVRMGAPSANSTYAGYIDRTLSREAAQEFYWGAQLPRLREVKKAWDPKDVFHNPQSVDPAE

SEQ ID NO: 171 - protein, 476 aa, Penicillium expansum
MGNTTSIGVVRDCLTSAVGGVAAHVAFQDTLLYQTSAVKPYNLNVPVTPAAVTYPQSANEVAAIVKCASDYDYKVQARSGGHSFGNFGLGGQNGAIVIDMKHFSQFSMDESTFIATIGPGTTLGNLDTELYNAGNRAMAHGICPSIRTGGHLTVGGLGPTSRQWGLALDHVEEVEVVLANSSVVRASDTQNQDVLFAIKGAAASFGIVTEFKVRTEEAPGLAVRYSYSFNLGTPAEKAKLAKDWQAYIAQENLTWKFSSNLIIFDGQIILEGIFFGSKEEYDKLNLEKKFPTSEPGTVLVITNWLGMIGHALEDTILRLIGDSPTWFYAKSLGFTPNTLIFDSTIDEFFDYIHKANAGTLAWSVMLSLEGGAINAVPKNATAYGHRDVLFWVQIFVVNPLGPISQTTYGFTDGLYNILARGVPESAGHAYLGCPDPKMPDAQRAYWRNNYPRLEELKRDLDPKDIFHNPQGVRVAS

SEQ ID NO: 172 - protein, 474 aa, Penicillium rubens
MGNTTSIAAGRDCLVSAVGPAHVTFQDALLYQTTAVDPYNLNIPVTPAAVTYPQSAEEIAAVVKCASDYDYKVQARSGGHSFGNYGLGGQNGAIVVDMKHFSQFSMDESTFVATIGPGTTLGDLDTELYNAGGRAMAHGICPTIRTGGHLTVGGLGPTARQWGLALDHIEEVEVVLANSSIVRASNTQNQDILFAVKGAAASFGIVTEFKVRTQEAPGLAVQYSFTFNLGSPAQKAKLVKDWQAFIAQENLSWKFYSNLVIFDGQIILEGIFFGSKEEYDELDLEKRFPTSEPGTVLVLTDWLGMIGHALEDTILKLVGDTPTWFYAKSLGFTPDTLIPDSAIDDFFDYIHKTNAGTLAWFVTLSLEGGAINSVSEDATAYGHRDVLFWFQVFVVNPLGPISQTTYDFTNGLYDVLAQAVPESAGHAYLGCPDPKMPDAQRAYWRSNLPRLEDLKGDLDPKDTFHNPQGVQVGP

SEQ ID NO: 173 - protein, 474 aa, Penicillium flavigenum
MGNTTSIAAGRDCLVSAVGPAHVTFQDALLYQTTAVDPYNLNIPVTPAAVTYPQSAEEIAAVVKCASDYDYKVQARSGGHSFGNYGLGGQNGAIVVEMKHFSQFSMDESTFVATIGPGTTLGDLDTELYNTGGRAMAHGICPTIRTGGHLTVGGLGPTARQWGLALDHIEEVEVVLANSSIVRASNTQNQDILFAVKGAAASFGIVTEFKVRTQEAPGLAVQYSFTFNLGSAAQKAKLVKDWQAFIAQENLSWKFYSNLVIFDGQIILEGIFFGSKEEYDELDLEKRFPTSEPGTVLVLTDWLGMIGHGLEDTILKLVGDTPTWFYAKSLGFTPDTLIPDSAIDDFFDYIHKTNAGTLAWFVTLSLEGGAINSVSEDATAYGHRDVLFWFQVFVVNPLGPISQTTYDFTNGLYDVLAQAVPESAGHAYLGCPDPKMPDAQRAYWRSNLPRLEDLKGDLDPKDTFHNPQGVQVGP

SEQ ID NO: 174 - protein, 9 aa, Artificial Sequence
  Synthetic
KVQARSGGH

SEQ ID NO: 175 - skipped

SEQ ID NO: 176 - protein, 15 aa, Artificial Sequence
  Synthetic
  misc_feature (10)..(10): Xaa is Val or Ile
  misc_feature (11)..(11): Xaa is Phe or Leu
  misc_feature (14)..(14): Xaa is Val or Ile
RASNTQNQDXXFAXK

SEQ ID NO: 177 - protein, 15 aa, Artificial Sequence
  Synthetic
RASNTQNQDVFFAVK

SEQ ID NO: 178 - protein, 15 aa, Artificial Sequence
  Synthetic
RASNTQNQDILFAVK

SEQ ID NO: 179 - protein, 15 aa, Artificial Sequence
  Synthetic
RASNTQNQDILFAIK

SEQ ID NO: 180 - protein, 15 aa, Artificial Sequence
  Synthetic
RASNTQNQDVLFAVK

SEQ ID NO: 181 - protein, 9 aa, Artificial Sequence
  Synthetic
  misc_feature (5)..(5): Xaa is Lys or Arg
CPTIXTGGH

SEQ ID NO: 182 - protein, 9 aa, Artificial Sequence
  Synthetic
CPTIKTGGH

SEQ ID NO: 183 - protein, 9 aa, Artificial Sequence
  Synthetic
CPTIRTGGH

SEQ ID NO: 184 - protein, 24 aa, Artificial Sequence
  Synthetic
  misc_feature (16)..(16): Xaa is Ala or Pro
  misc_feature (23)..(23): Xaa is Ala or Gly
WFVTLSLEGGAINDVXEDATAYXH

SEQ ID NO: 185 - protein, 24 aa, Artificial Sequence
  Synthetic
WFVTLSLEGGAINDVAEDATAYAH

SEQ ID NO: 186 - protein, 37 aa, Artificial Sequence
  Synthetic
  misc_feature (2)..(2): Xaa is Ile or Val
  misc_feature (4)..(4): Xaa is Asp, Gln, or Glu
  misc_feature (8)..(8): Xaa is Glu, Asp, or Gly
  misc_feature (10)..(10): Xaa is Thr or Ala
  misc_feature (19)..(19): Xaa is Arg, Gln, or Lys
  misc_feature (25)..(25): Xaa is Val or Ala
  misc_feature (36)..(36): Xaa is Arg or Lys
PXSXTTYXFXDGLYDVLAXAVPESXGHAYLGCPDPXM

SEQ ID NO: 187 - protein, 37 aa, Artificial Sequence
  Synthetic
PISDTTYEFTDGLYDVLARAVPESVGHAYLGCPDPRM

SEQ ID NO: 188 - protein, 37 aa, Artificial Sequence
  Synthetic
PISETTYEFTDGLYDVLARAVPESVGHAYLGCPDPRM

SEQ ID NO: 189 - protein, 9 aa, Artificial Sequence
  Synthetic
  misc_feature (5)..(5): Xaa is Thr, Asn, or Ser
MKHFXQFSM

SEQ ID NO: 190 - protein, 9 aa, Artificial Sequence
  Synthetic
MKHFTQFSM

SEQ ID NO: 191 - protein, 9 aa, Artificial Sequence
  Synthetic
MKHFSQFSM

SEQ ID NO: 192 - protein, 9 aa, Artificial Sequence
  Synthetic
MKHFNQFSM

SEQ ID NO: 193 - protein, 13 aa, Artificial Sequence
  Synthetic
  misc_feature (2)..(2): Xaa is Glu or Gln
  misc_feature (3)..(3): Xaa is Thr or Ser
  misc_feature (5)..(5): Xaa is Glu, Ala, or Asp
  misc_feature (6)..(6): Xaa is Gln or Glu
  misc_feature (9)..(9): Xaa is Gly or Ala
  misc_feature (10)..(10): Xaa is Val or Ile
PXXAXXIAXXVKC

SEQ ID NO: 194 - protein, 13 aa, Artificial Sequence
  Synthetic
PETAEQIAGIVKC

SEQ ID NO: 195 - protein, 13 aa, Artificial Sequence
  Synthetic
PQSADEIAAVVKC

SEQ ID NO: 196 - protein, 13 aa, Artificial Sequence
  Synthetic
PETAAQIAGVVKC

SEQ ID NO: 197 - protein, 13 aa, Artificial Sequence
  Synthetic
PQSAEEIAAVVKC

SEQ ID NO: 198 - protein, 13 aa, Artificial Sequence
  Synthetic
PETAEQIAGVVKC

SEQ ID NO: 199 - protein, 13 aa, Artificial Sequence
  Synthetic
PETAEQIAAVVKC

SEQ ID NO: 200 - protein, 23 aa, Artificial Sequence
  Synthetic
  misc_feature (5)..(5): Xaa is Ile or Val
  misc_feature (8)..(8): Xaa is Leu or Val
  misc_feature (12)..(12): Xaa is Ser or Ala
  misc_feature (14)..(14): Xaa is Leu or His
  misc_feature (15)..(15): Xaa is Ala or Val
  misc_feature (16)..(16): Xaa is Ala or Val
  misc_feature (18)..(18): Xaa is Pro or Gln
  misc_feature (19)..(19): Xaa is Asn or Asp
  misc_feature (20)..(20): Xaa is Gln or Glu
  misc_feature (23)..(23): Xaa is Trp or Tyr
RDCLXSAXGGNXAXXXFXXXLLX

SEQ ID NO: 201 - protein, 23 aa, Artificial Sequence
  Synthetic
RDCLISAVGGNAAHVAFQDQLLY

SEQ ID NO: 202 - protein, 23 aa, Artificial Sequence
  Synthetic
RDCLISALGGNSALAVFPNELLW

SEQ ID NO: 203 - protein, 23 aa, Artificial Sequence
  Synthetic
RDCLISALGGNSALAAFPNELLW

SEQ ID NO: 204 - protein, 23 aa, Artificial Sequence
  Synthetic
RDCLISALGGNSALAVFPNQLLW

SEQ ID NO: 205 - protein, 23 aa, Artificial Sequence
  Synthetic
RDCLISALGGNSALAAFPNQLLW

SEQ ID NO: 206 - protein, 23 aa, Artificial Sequence
  Synthetic
RDCLVSALGGNSALAAFPNQLLW

SEQ ID NO: 207 - protein, 14 aa, Artificial Sequence
  Synthetic
  misc_feature (3)..(3): Xaa is Glu or Gln
  misc_feature (4)..(4): Xaa is Pro or Gln
RTXXAPGLAVQYSY

SEQ ID NO: 208 - protein, 14 aa, Artificial Sequence
  Synthetic
RTEPAPGLAVQYSY

SEQ ID NO: 209 - protein, 14 aa, Artificial Sequence
  Synthetic
RTEQAPGLAVQYSY

SEQ ID NO: 210 - protein, 14 aa, Artificial Sequence
  Synthetic
RTQPAPGLAVQYSY

SEQ ID NO: 211 - protein, 18 aa, Artificial Sequence
  Synthetic
  misc_feature (3)..(3): Xaa is Ser or Ala
  misc_feature (6)..(6): Xaa is Ser or Ala
  misc_feature (7)..(7): Xaa is Ala or Gln
  misc_feature (8)..(8): Xaa is Lys or Glu
  misc_feature (12)..(12): Xaa is Arg or Trp
  misc_feature (13)..(13): Xaa is Gln or Lys
  misc_feature (16)..(16): Xaa is Asn, Ser, or Thr
WQXFIXXXNLTXXFYXNM

SEQ ID NO: 212 - protein, 18 aa, Artificial Sequence
  Synthetic
WQSFISAKNLTRQFYNNM

SEQ ID NO: 213 - protein, 18 aa, Artificial Sequence
  Synthetic
WQSFISAKNLTRQFYTNM

SEQ ID NO: 214 - protein, 517 aa, C. sativa
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 215 - protein, 516 aa, C. sativa
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

(19) United States
(12) Patent Application Publication
(10) Pub. No.: US 2023/0137139 A1
(43) Pub. Date: May 4, 2023

(54) BIOSYNTHESIS OF CANNABINOIDS AND CANNABINOID PRECURSORS

(71) Applicant: Ginkgo Bioworks, Inc., Boston, MA (US)

(72) Inventors: Kim Cecelia Anderson, Somerville, MA (US); Jeffrey lan Boucher, Allston, MA (US); Elena Brevnova, Boston, MA (US); Dylan Alexander Carlin, Jamaica Plain, MA (US); Brian Carvalho, Somerville, MA (US); Nicholas Flores, Boston, MA (US); Katrina Forrest, Boston, MA (US); Gabriel Rodriguez, Central, SC (US); Michelle Spencer, Boston, MA (US)

(73) Assignee: Ginkgo Bioworks, Inc., Boston, MA (US)

(21) Appl. No.: 17/914,060

(22) PCT Filed: Mar. 26, 2021
(86) PCT No.: PCT/US2021/024398

§ 371 Date: Sep. 23, 2022

Related U.S. Application Data
(60) Provisional application No. 63/000,419, filed on Mar. 26, 2020.

(51) Int. Cl. C07D 311/80; C12N 15/70; C12N 15/81; C07C 65/19; C12P 7/42; C12P 17/06; C12N 9/02

(52) U.S. Cl. CPC C07D 311/80; C12N 15/70; C12N 15/81; C07C 65/19; C12P 7/42; C12P 17/06; C12N 9/0004; C12Y 121/03007; C12Y 121/03008

(57) ABSTRACT

Aspects of the disclosure relate to biosynthesis of cannabinoids and cannabinoid precursors in recombinant cells and in vitro.

### CROSS REFERENCE TO RELATED APPLICATION

This application claims the benefit under 35 U.S.C. § 119(e) of U.S. Provisional Application No. 63/000,419, filed Mar. 26, 2020, entitled “BIOSYNTHESIS OF CANNABINOIDS AND CANNABINOID PRECURSORS,” the entire disclosure of which is hereby incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. The ASCII file, created on Mar. 24, 2021, is named G091970059WO00-SEQ-OMJ.txt and is 526 kilobytes in size.

### FIELD OF INVENTION

The present disclosure relates to the biosynthesis of cannabinoids and cannabinoid precursors, such as in recombinant cells.

### BACKGROUND

Cannabinoids are chemical compounds that may act as ligands for endocannabinoid receptors and have multiple medical applications. Traditionally, cannabinoids have been isolated from plants of the genus Cannabis. The use of plants for producing cannabinoids is inefficient, however, with isolated products often limited to the two most prevalent endogenous cannabinoids, THC and CBD, as other cannabinoids are typically produced in very low concentrations in Cannabis plants. Further, the cultivation of Cannabis plants is restricted in many jurisdictions. In addition, in order to obtain consistent results, Cannabis plants are often grown in a controlled environment, such as indoor grow rooms without windows, to provide flexibility in modulating growing conditions such as lighting, temperature, humidity, airflow, etc. Growing Cannabis plants in such controlled environments can result in high energy usage per gram of cannabinoid produced, especially for rare cannabinoids that the plants produce only in small amounts. For example, lighting in such grow rooms is provided by artificial sources, such as high-powered sodium lights. As many species of Cannabis have a vegetative cycle that requires 18 or more hours of light per day, powering such lights can result in significant energy expenditures. It has been estimated that between 0.88-1.34 kWh of energy is required to produce one gram of THC in dried Cannabis flower form (e.g., before any extraction or purification). Additionally, concern has been raised over agricultural practices in certain jurisdictions, such as California, where the growing season coincides with the dry season such that the water usage may impact connected surface water in streams (Dillis, Christopher, Connor McIntee, Van Butsic, Lance Le, Kason Grady, and Theodore Grantham. “Water storage and irrigation practices for cannabis drive seasonal patterns of water extraction and use in Northern California.” Journal of Environmental Management 272 (2020): 110955).

Cannabinoids can be produced through chemical synthesis (see, e.g., U.S. Pat. No. 7,323,576 to Souza et al). However, such methods suffer from low yields and high cost. Production of cannabinoids, cannabinoid analogs, and cannabinoid precursors using engineered organisms may provide an advantageous approach to meet the increasing demand for these compounds.

### SUMMARY

Aspects of the present disclosure provide methods for production of cannabinoids and cannabinoid precursors from fatty acid substrates using genetically modified host cells.

Aspects of the disclosure relate to host cells that comprise a heterologous polynucleotide encoding a terminal synthase (TS), wherein the TS comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical, or is 100% identical, to SEQ ID NO: 27 or 25 and wherein the host cell is capable of producing at least one cannabinoid.

Aspects of the disclosure relate to host cells that comprise a heterologous polynucleotide encoding a terminal synthase (TS), wherein the TS comprises a sequence that is at least 90% identical to SEQ ID NO: 27 or 25 and wherein the host cell is capable of producing at least one cannabinoid.

In some embodiments, relative to the sequence of SEQ ID NO: 27, the TS comprises an amino acid substitution at a residue corresponding to position 33, 39, 55, 57, 61, 62, 63, 71, 112, 122, 126, 129, 131 180, 183, 202, 256, 257, 260, 287, 295, 341, 386, 392, 394, 398, 410, 423, 426, 450, and/or 472 of SEQ ID NO: 27.

In some embodiments, the TS comprises: the amino acid D at a residue corresponding to position 33 in SEQ ID NO: 27; the amino acid F at a residue corresponding to position 39 in SEQ ID NO: 27; the amino acid S at a residue corresponding to position 55 in SEQ ID NO: 27; the amino acid Q or E at a residue corresponding to position 57 in SEQ ID NO: 27; the amino acid A at a residue corresponding to position 61 in SEQ ID NO: 27; the amino acid I at a residue corresponding to position 62 in SEQ ID NO: 27; the amino acid I at a residue corresponding to position 63 in SEQ ID NO: 27; the amino acid I at a residue corresponding to position 71 in SEQ ID NO: 27; the amino acid V or T at a residue corresponding to position 112 in SEQ ID NO: 27; the amino acid S, G, A or E at a residue corresponding to position 122 in SEQ ID NO: 27; the amino acid A, R, T, K, or D at a residue corresponding to position 126 in SEQ ID NO: 27; the amino acid W at a residue corresponding to position 129 in SEQ ID NO: 27; the amino acid S at a residue corresponding to position 131 in SEQ ID NO: 27; the amino acid T at a residue corresponding to position 180 in SEQ ID NO: 27; the amino acid T at a residue corresponding to position 183 in SEQ ID NO: 27; the amino acid S or G at a residue corresponding to position 202 in SEQ ID NO: 27; the amino acid F or M at a residue corresponding to position 256 in SEQ ID NO: 27; the amino acid S at a residue corresponding to position 257 in SEQ ID NO: 27; the amino acid M or F at a residue corresponding to position 260 in SEQ ID NO: 27; the amino acid R at a residue corresponding to position 287 in SEQ ID NO: 27; the amino acid S at a residue corresponding to position 295 in SEQ ID NO: 27; the amino acid S at a residue corresponding to position 341 in SEQ ID NO: 27; the amino acid A at a residue corresponding to position 386 in SEQ ID NO: 27; the amino acid H at a residue corresponding to position 392 in SEQ ID NO: 27; the amino acid T at a residue corresponding to position 394 in SEQ ID NO: 27; the amino acid F, T, A, or L at a residue corresponding to position 398 in SEQ ID NO: 27; the amino acid N at a residue corresponding to position 410 in SEQ ID NO: 27; the amino acid A at a residue corresponding to position 423 in SEQ ID NO: 27; the amino acid Y at a residue corresponding to position 426 in SEQ ID NO: 27; the amino acid K at a residue corresponding to position 450 in SEQ ID NO: 27; and/or the amino acid R or A at a residue corresponding to position 472 in SEQ ID NO: 27.

In some embodiments, the TS comprises one or more of the following amino acid substitutions relative to the sequence of SEQ ID NO: 27: T33D; Y39F; T55S; A57Q; A57E; G61A; V62I; V63I; Y71I; E112V; E112T; N122S; N122G; N122A; N122E; I126A; I126R; I126T; I126K; I126D; Y129W; N131S; S180T; R183T; N202S; N202G; Y256F; Y256M; N257S; V260M; V260F; H287R; N295S; A341S; V386A; L392H; M394T; V398F; V398T; V398A; V398L; D410N; S423A; H426Y; R450K; P472R; and/or P472A.

In some embodiments, the cannabinoid is a CBC-type cannabinoid. In some embodiments, the cannabinoid is cannabichromenic acid (CBCA) and/or cannabichromevarinic acid (CBCVA). In some embodiments, the host cell further produces one or more of tetrahydrocannabinolic acid (THCA), cannabidiolic acid (CBDA) and/or tetrahydrocannabivarinic acid (THCVA).

In some embodiments, the TS produces a higher ratio of CBCA:CBDA, CBCA:THCA, and/or CBCVA:THCVA than a control TS. In some embodiments, the control TS is a TS comprising the sequence of SEQ ID NO: 20, 23, 25 or 27. In some embodiments, the TS comprises one or more of the following amino acid substitutions relative to SEQ ID NO: 27: A57Q and G61A; Y71I; and/or V260F. In some embodiments, the TS has a higher product specificity for a CBC-type cannabinoid than a control TS. In some embodiments, the control TS is a TS comprising the sequence of SEQ ID NO: 20, 23, 25 or 27. In some embodiments, the TS comprises Y39F and/or V63I relative to the sequence of SEQ ID NO: 27.

In some embodiments, the TS comprises the sequence of any one of SEQ ID NOs: 25, 27, 105, 126, 134, 155, 162, 164, or 165, optionally wherein relative to the sequence of SEQ ID NO: 27, the TS comprises an amino acid substitution at a residue corresponding to position 33, 39, 55, 57, 61, 62, 63, 71, 112, 122, 126, 129, 131 180, 183, 202, 256, 257, 260, 287, 295, 341, 386, 392, 394, 398, 410, 423, 426, 450, and/or 472 of SEQ ID NO: 27. In some embodiments, the sequence of the TS comprises one or more of the following motifs: KVQARSGGH (SEQ ID NO: 174); RASNTQNQD[VI][FL]FA[VI]K (SEQ ID NO: 176); CPTI[KR]TGGH (SEQ ID NO: 181); WFVTLSLEGGAINDV[AP]EDATAY[AG]H (SEQ ID NO: 184); P[IV]S[DQE]TTY[EDG]F[TA]DGLYDVLA[RQK]AVPES[VA]GHAYLGCPDP[RK]M (SEQ ID NO: 186); MKHF[TNS]QFSM (SEQ ID NO: 189); P[EQ][TS]A[EAD][QE]IA[GA][VI]VKC (SEQ ID NO: 193); RDCL[IV]SA[LV]GGN[SA]A[LH][AV][AV]F[PQ][ND][QE]LL[WY] (SEQ ID NO: 200); RT[EQ][PQ]APGLAVQYSY (SEQ ID NO: 207); and/or WQ[SA]FI[SA][AQ][KE]NLT[RW][QK]FY[NST]NM (SEQ ID NO: 211).

Further aspects of the disclosure relate to host cells for producing a cannabinoid, wherein the host cell comprises a heterologous polynucleotide encoding a terminal synthase (TS), wherein the sequence of the TS comprises one or more of the following motifs: KVQARSGGH (SEQ ID NO: 174); RASNTQNQD[VI][FL]FA[VI]K (SEQ ID NO: 176); CPTI[KR]TGGH (SEQ ID NO: 181); WFVTLSLEGGAINDV[AP]EDATAY[AG]H (SEQ ID NO: 184); P[IV]S[DQE]TTY[EDG]F[TA]DGLYDVLA[RQK]AVPES[VA]GHAYLGCP DP[RK]M (SEQ ID NO: 186); MKHF[TNS]QFSM (SEQ ID NO: 189); P[EQ][TS]A[EAD][QE]IA[GA][VI]VKC (SEQ ID NO: 193); RDCL[IV]SA[LV]GGN[SA]A[LH][AV][AV]F[PQ][ND][QE]LL[WY] (SEQ ID NO: 200); RT[EQ][PQ]APGLAVQYSY (SEQ ID NO: 207); and/or WQ[SA]FI[SA][AQ][KE]NLT[RW][QK]FY[NST]NM (SEQ ID NO: 211), and wherein the host cell is capable of producing at least one cannabinoid.

In some embodiments, the motif KVQARSGGH (SEQ ID NO: 174) is located at residues in the TS corresponding to residues 72-80 in SEQ ID NO: 27; the motif RASNTQNQD[VI][FL]FA[VI]K (SEQ ID NO: 176) is located at residues in the TS corresponding to residues 183-197 in SEQ ID NO: 27; the motif CPTI[KR]TGGH (SEQ ID NO: 181) is located at residues in the TS corresponding to residues 141-149 in SEQ ID NO: 27; the motif WFVTLSLEGGAINDV[AP]EDATAY[AG]H (SEQ ID NO: 184) is located at residues in the TS corresponding to residues 360-383 in SEQ ID NO: 27; the motif P[IV]S[DQE]TTY[EDG]F[TA]DGLYDVLA[RQK]AVPES[VA]GHAYLGCPDP[RK]M (SEQ ID NO: 186) is located at residues in the TS corresponding to residues 400-436 in SEQ ID NO: 27; the motif MKHF[TNS]QFSM (SEQ ID NO: 189) is located at residues in the TS corresponding to residues 98-106 in SEQ ID NO: 27; the motif P[EQ][TS]A[EAD][QE]IA[GA][VI]VKC (SEQ ID NO: 193) is located at residues in the TS corresponding to residues 53-65 in SEQ ID NO: 27; the motif RDCL[IV]SA[LV]GGN[SA]A[LH][AV][AV]F[PQ][ND][QE]LL[WY] (SEQ ID NO: 200) is located at residues in the TS corresponding to residues 10-32 in SEQ ID NO: 27; the motif RT[EQ][PQ]APGLAVQYSY (SEQ ID NO: 207) is located at residues in the TS corresponding to residues 212-225 in SEQ ID NO: 27; and/or the motif WQ[SA]FI[SA][AQ][KE]NLT[RW][QK]FY[NST]NM (SEQ ID NO: 211) is located at residues in the TS corresponding to residues 242-259 in SEQ ID NO: 27.

In some embodiments, the TS is a fungal TS or a conservatively substituted version thereof. In some embodiments, the TS is an Apergillus TS or a conservatively substituted version thereof. In some embodiments, the TS comprises a sequence that is at least 90% identical to any one of SEQ ID NOs: 25, 27, 105, 112, 126, 130, 134, 144, 155, 159, 162-167, or 172. In some embodiments, relative to the sequence of SEQ ID NO: 27, the TS comprises an amino acid substitution at a residue corresponding to position 33, 39, 55, 57, 61, 62, 63, 71, 112, 122, 126, 129, 131 180, 183, 202, 256, 257, 260, 287, 295, 341, 386, 392, 394, 398, 410, 423, 426, 450, and/or 472 of SEQ ID NO: 27. In some embodiments, the TS comprises: the amino acid D at a residue corresponding to position 33 in SEQ ID NO: 27; the amino acid F at a residue corresponding to position 39 in SEQ ID NO: 27; the amino acid S at a residue corresponding to position 55 in SEQ ID NO: 27; the amino acid Q or E at a residue corresponding to position 57 in SEQ ID NO: 27; the amino acid A at a residue corresponding to position 61 in SEQ ID NO: 27; the amino acid I at a residue corresponding to position 62 in SEQ ID NO: 27; the amino acid I at a residue corresponding to position 63 in SEQ ID NO: 27; the amino acid I at a residue corresponding to position 71 in SEQ ID NO: 27; the amino acid V or T at a residue corresponding to position 112 in SEQ ID NO: 27; the amino acid S, G, A or E at a residue corresponding to position 122 in SEQ ID NO: 27; the amino acid A, R, T, K, or D at a residue corresponding to position 126 in SEQ ID NO: 27; the amino acid W at a residue corresponding to position 129 in SEQ ID NO: 27; the amino acid S at a residue corresponding to position 131 in SEQ ID NO: 27; the amino acid T at a residue corresponding to position 180 in SEQ ID NO: 27; the amino acid T at a residue corresponding to position 183 in SEQ ID NO: 27; the amino acid S or G at a residue corresponding to position 202 in SEQ ID NO: 27; the amino acid F or M at a residue corresponding to position 256 in SEQ ID NO: 27; the amino acid S at a residue corresponding to position 257 in SEQ ID NO: 27; the amino acid M or F at a residue corresponding to position 260 in SEQ ID NO: 27; the amino acid R at a residue corresponding to position 287 in SEQ ID NO: 27; the amino acid S at a residue corresponding to position 295 in SEQ ID NO: 27; the amino acid S at a residue corresponding to position 341 in SEQ ID NO: 27; the amino acid A at a residue corresponding to position 386 in SEQ ID NO: 27; the amino acid H at a residue corresponding to position 392 in SEQ ID NO: 27; the amino acid T at a residue corresponding to position 394 in SEQ ID NO: 27; the amino acid F, T, A, or L at a residue corresponding to position 398 in SEQ ID NO: 27; the amino acid N at a residue corresponding to position 410 in SEQ ID NO: 27; the amino acid A at a residue corresponding to position 423 in SEQ ID NO: 27; the amino acid Y at a residue corresponding to position 426 in SEQ ID NO: 27; the amino acid K at a residue corresponding to position 450 in SEQ ID NO: 27; and/or the amino acid R or A at a residue corresponding to position 472 in SEQ ID NO: 27.

In some embodiments, the TS comprises one or more of the following amino acid substitutions relative to the sequence of SEQ ID NO: 27: T33D; Y39F; T55S; A57Q; A57E; G61A; V62I; V63I; Y71I; E112V; E112T; N122S; N122G; N122A; N122E; I126A; I126R; I126T; I126K; I126D; Y129W; N131S; S180T; R183T; N202S; N202G; Y256F; Y256M; N257S; V260M; V260F; H287R; N295S; A341S; V386A; L392H; M394T; V398F; V398T; V398A; V398L; D410N; S423A; H426Y; R450K; P472R; and/or P472A. In some embodiments, the TS comprises the sequence of any one of SEQ ID NOs: 25, 27, 105, 112, 126, 130, 134, 143, 144, 155, 159, 162-167, or 172 or a conservatively substituted version thereof.

Further aspects of the disclosure relate to host cells that comprises a heterologous polynucleotide encoding a terminal synthase (TS), wherein the TS comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical, or is 100% identical, to any one of SEQ ID NOs: 25, 27, 105, 112, 126, 130, 134, 144, 155, 159, 162-167, or 172, wherein the host cell is capable of producing at least one cannabinoid.

Further aspects of the disclosure relate to host cells that comprises a heterologous polynucleotide encoding a terminal synthase (TS), wherein the TS comprises a sequence that is at least 90% identical to any one of SEQ ID NOs: 25, 27, 105, 112, 126, 130, 134, 144, 155, 159, 162-167, or 172, wherein the host cell is capable of producing at least one cannabinoid.

In some embodiments, the sequence that is at least 90% identical to any one of SEQ ID NOs: 25, 27, 105, 112, 126, 130, 134, 144, 155, 159, 162-167, or 172 is linked to one or more signal peptides. In some embodiments, the sequence that is at least 90% identical to any one of SEQ ID NOs: 25, 27, 105, 112, 126, 130, 134, 144, 155, 159, 162-167, or 172 is linked to a signal peptide that comprises SEQ ID NO: 16 or a sequence that has no more than two amino acid substitutions, insertions, additions, or deletions relative to the sequence of SEQ ID NO: 16. In some embodiments, the signal peptide is linked to the N-terminus of the sequence that is at least 90% identical to any one of SEQ ID NOs: 25, 27, 105, 112, 126, 130, 134, 144, 155, 159, 162-167, or 172. In some embodiments, an N-terminal methionine is removed from SEQ ID NOs: 27, 105, 112, 126, 130, 134, 144, 155, 159, 162-167, or 172 and wherein a methionine residue is added to the N-terminus of the signal peptide. In some embodiments, the sequence that is at least 90% identical to any one of SEQ ID NOs: 25, 27, 105, 112, 126, 130, 134, 144, 155, 159, 162-167, or 172 is linked to a signal peptide that comprises SEQ ID NO: 17 or a sequence that has no more than one amino acid substitution, insertion, addition, or deletion relative to the sequence of SEQ ID NO: 17. In some embodiments, the signal peptide that comprises SEQ ID NO: 17 or a sequence that has no more than one amino acid substitution, insertion, addition, or deletion relative to the sequence of SEQ ID NO: 17 is linked to the C-terminus of the sequence that is at least 90% identical to any one of SEQ ID NOs: 25, 27, 105, 112, 126, 130, 134, 144, 155, 159, 162-167, or 172.

In some embodiments, relative to the sequence of SEQ ID NO: 27, the TS comprises an amino acid substitution at a residue corresponding to position 33, 39, 55, 57, 61, 62, 63, 71, 112, 122, 126, 129, 131 180, 183, 202, 256, 257, 260, 287, 295, 341, 386, 392, 394, 398, 410, 423, 426, 450, and/or 472 of SEQ ID NO: 27. In some embodiments, the TS comprises: the amino acid D at a residue corresponding to position 33 in SEQ ID NO: 27; the amino acid F at a residue corresponding to position 39 in SEQ ID NO: 27; the amino acid S at a residue corresponding to position 55 in SEQ ID NO: 27; the amino acid Q or E at a residue corresponding to position 57 in SEQ ID NO: 27; the amino acid A at a residue corresponding to position 61 in SEQ ID NO: 27; the amino acid I at a residue corresponding to position 62 in SEQ ID NO: 27; the amino acid I at a residue corresponding to position 63 in SEQ ID NO: 27; the amino acid I at a residue corresponding to position 71 in SEQ ID NO: 27; the amino acid V or T at a residue corresponding to position 112 in SEQ ID NO: 27; the amino acid S, G, A or E at a residue corresponding to position 122 in SEQ ID NO: 27; the amino acid A, R, T, K, or D at a residue corresponding to position 126 in SEQ ID NO: 27; the amino acid W at a residue corresponding to position 129 in SEQ ID NO: 27; the amino acid S at a residue corresponding to position 131 in SEQ ID NO: 27; the amino acid T at a residue corresponding to position 180 in SEQ ID NO: 27; the amino acid T at a residue corresponding to position 183 in SEQ ID NO: 27; the amino acid S or G at a residue corresponding to position 202 in SEQ ID NO: 27; the amino acid F or M at a residue corresponding to position 256 in SEQ ID NO: 27; the amino acid S at a residue corresponding to position 257 in SEQ ID NO: 27; the amino acid M or F at a residue corresponding to position 260 in SEQ ID NO: 27; the amino acid R at a residue corresponding to position 287 in SEQ ID NO: 27; the amino acid S at a residue corresponding to position 295 in SEQ ID NO: 27; the amino acid S at a residue corresponding to position 341 in SEQ ID NO: 27; the amino acid A at a residue corresponding to position 386 in SEQ ID NO: 27; the amino acid H at a residue corresponding to position 392 in SEQ ID NO: 27; the amino acid T at a residue corresponding to position 394 in SEQ ID NO: 27; the amino acid F, T, A, or L at a residue corresponding to position 398 in SEQ ID NO: 27; the amino acid N at a residue corresponding to position 410 in SEQ ID NO: 27; the amino acid A at a residue corresponding to position 423 in SEQ ID NO: 27; the amino acid Y at a residue corresponding to position 426 in SEQ ID NO: 27; the amino acid K at a residue corresponding to position 450 in SEQ ID NO: 27; and/or the amino acid R or A at a residue corresponding to position 472 in SEQ ID NO: 27. In some embodiments, the TS comprises one or more of the following amino acid substitutions relative to the sequence of SEQ ID NO: 27: T33D; Y39F; T55S; A57Q; A57E; G61A; V62I; V63I; Y71I; E112V; E112T; N122S; N122G; N122A; N122E; I126A; I126R; I126T; I126K; I126D; Y129W; N131S; S180T; R183T; N202S; N202G; Y256F; Y256M; N257S; V260M; V260F; H287R; N295S; A341S; V386A; L392H; M394T; V398F; V398T; V398A; V398L; D410N; S423A; H426Y; R450K; P472R; and/or P472A.

In some embodiments, the heterologous polynucleotide comprises a sequence that is at least 90% identical to any one of SEQ ID NOs: 26, 28, 35, 42, 56, 60, 64, 74, 85, 89, 92, 93, 94, 95, 96, 97, and 102. In some embodiments, the TS sequence comprises any one of SEQ ID NOs: 25, 27, 105, 112, 126, 130, 134, 144, 155, 159, 162-167 and 172.

Further aspects of the disclosure relate to host cells that comprise a heterologous polynucleotide encoding a terminal synthase (TS), wherein the TS comprises a sequence that is at least 90% identical to any one of SEQ ID NOs: 25, 27, 105, 112, 126, 130, 134, 144, 155, 159, 162-167, or 172, or wherein the host cell comprises a conservatively substituted version of any one of SEQ ID NOs: 25, 27, 105, 112, 126, 130, 134, 144, 155, 159, 162-167, or 172.

Further aspects of the disclosure relate to host cells that comprise a heterologous polynucleotide encoding a terminal synthase (TS), wherein the host cell is capable of producing at least one cannabinoid, and wherein the TS is a fungal TS or a conservatively substituted version thereof. In some embodiments, the fungal TS is an Aspergillus TS or a conservatively substituted version thereof. In some embodiments, the cannabinoid is a is a CBC-type cannabinoid. In some embodiments, the cannabinoid is cannabichromenic acid (CBCA) and/or cannabichromevarinic acid (CBCVA). In some embodiments, the host cell further produces one or more of tetrahydrocannabinolic acid (THCA), cannabidiolic acid (CBDA) and/or tetrahydrocannabivarinic acid (THCVA).

In some embodiments, the host cell is a plant cell, an algal cell, a yeast cell, a bacterial cell, or an animal cell. In some embodiments, the host cell is a yeast cell. In some embodiments, the yeast cell is a Saccharomyces cell, a Yarrowia cell, a Komagataella cell, or a Pichia cell. In some embodiments, the Saccharomyces cell is a Saccharomyces cerevisiae cell. In some embodiments, the host cell is a bacterial cell. In some embodiments, the bacterial cell is an E. coli cell. In some embodiments, the host cell further comprises one or more heterologous polynucleotides encoding one or more of: an acyl activating enzyme (AAE), a polyketide synthase (PKS), a polyketide cyclase (PKC), a prenyltransferase (PT), and/or an additional terminal synthase (TS). In some embodiments, the PKS is an olivetol synthase (OLS) or a divarinol synthase. Further aspects of the disclosure relate to methods comprising culturing any of the host cells associated with the disclosure.

Further aspects of the disclosure relate to methods for producing a cannabinoid comprising contacting a CBG-type cannabinoid with a terminal synthase (TS), wherein the TS comprises a sequence that is at least 90% identical to any one of SEQ ID NOs: 25, 27, 105, 112, 126, 130, 134, 144, 155, 159, 162-167, or 172. In some embodiments, contacting the CBG-type cannabinoid with the TS occurs in vitro. In some embodiments, contacting the CBG-type cannabinoid with the TS occurs in vivo. In some embodiments, contacting the CBG-type cannabinoid with the TS occurs in a host cell. Further aspects of the disclosure relate to methods for producing a cannabinoid comprising contacting a CBG-type cannabinoid in vivo with an oxidative cyclization catalyst adapted to preferentially convert the CBG-type cannabinoid to a CBC-type cannabinoid as compared to a CBD-type cannabinoid, a THC-type cannabinoid or both.

In some embodiments, the cannabinoid is a cyclized product of a CBG-type cannabinoid. In some embodiments, the cannabinoid is a cannabinoid with a cyclized prenyl moiety. In some embodiments, the cannabinoid is a CBC-type cannabinoid, a CBD-type cannabinoid, or a THC-type cannabinoid. In some embodiments, the cannabinoid is a CBC-type cannabinoid. In some embodiments, the CBG-type cannabinoid is cannabigerolic acid. In some embodiments, the CBC-type cannabinoid is CBCA. In some embodiments, the TS comprises the sequence of any one of SEQ ID NOs: 25, 27, 105, 112, 126, 130, 134, 144, 155, 159, 162-167, or 172 or a conservatively substituted version thereof.

Further aspects of the disclosure relate to host cells comprising a CBG-type cannabinoid and a means for catalyzing the oxidative cyclization of the CBG-type cannabinoid to preferentially convert the CBG-type cannabinoid to a CBC-type cannabinoid as compared to a CBG-type cannabinoid, a THC-type cannabinoid, or both. Further aspects of the disclosure relate to host cells comprising a CBG-type cannabinoid and an oxidative cyclization catalyst adapted to preferentially convert the CBG-type cannabinoid to a CBC-type cannabinoid as compared to a CBG-type cannabinoid, a THC-type cannabinoid, or both. In some embodiments, the means for catalyzing the oxidative cyclization of the CBG-type cannabinoid to produce a CBC-type cannabinoid is a heterologous polynucleotide encoding a terminal synthase (TS), wherein the TS comprises a sequence that is at least 90% identical to any of SEQ ID NOs: 25, 27, 105, 112, 126, 130, 134, 144, 155, 159, 162-167, or 172 or a conservatively substituted version thereof. In some embodiments, the TS is also capable of producing THCA, THCVA or CBDA.

Further aspects of the disclosure relate to non-naturally occurring nucleic acid encoding a terminal synthase (TS), wherein the non-naturally occurring nucleic acid comprises a sequence that has at least 90% identity to any one of SEQ ID NOs: 26, 28, 35, 42, 56, 60, 64, 74, 85, 89, 92, 93, 94, 95, 96, 97, and 102. Further aspects of the disclosure relate to vectors comprising non-naturally occurring nucleic acids associated with the disclosure. Further aspects of the disclosure relate to expression cassettes comprising non-naturally occurring nucleic acids associated with the disclosure. Further aspects of the disclosure relate to host cells transformed with non-naturally occurring nucleic acids, vectors, or expression cassettes associated with the disclosure.

Further aspects of the disclosure relate to bioreactors for producing a cannabinoid, wherein the bioreactor contains a CBG-type cannabinoid and a terminal synthase (TS), wherein the TS comprises a sequence that is at least 90% identical to any one of SEQ ID NOs: 25, 27, 105, 112, 126, 130, 134, 144, 155, 159, 162-167, or 172 or wherein the TS comprises a conservatively substituted version of any one of SEQ ID NOs: 25, 27, 105, 112, 126, 130, 134, 144, 155, 159, 162-167, or 172.

Further aspects of the disclosure relate to non-naturally occurring terminal synthases (TS), wherein the TS comprises a sequence that is at least 90% identical to any one of SEQ ID NOs: 25, 27, 105, 112, 126, 130, 134, 144, 155, 159, 162-167, or 172.

Further aspects of the disclosure relate to oxidative cyclization catalysts adapted to preferentially convert a CBG-type cannabinoid to a CBC-type compound in vivo as compared to a THC-type compound or a CBD-type compound.

Each of the limitations of the invention can encompass various embodiments of the invention. It is, therefore, anticipated that each of the limitations of the invention involving any one element or combinations of elements can be included in each aspect of the invention. This disclosure is not limited in its application to the details of construction and the arrangement of components set forth in the following description or illustrated in the drawings. The invention is capable of other embodiments and of being practiced or of being carried out in various ways. Also, the phraseology and terminology used in this application is for the purpose of description and should not be regarded as limiting. The use of “including,” “comprising,” or “having,” “containing,” “involving,” and variations thereof, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic depicting the native Cannabis biosynthetic pathway for production of cannabinoid compounds, including five enzymatic steps mediated by: (Ria) acyl activating enzymes (AAE); (R2a) olivetol synthase enzymes (OLS); (R3a) olivetolic acid cyclase enzymes (OAC); (R4a) prenyltransferase enzymes (PT); and (R5a) terminal synthase enzymes (TS). Formulae 1a-11a correspond to hexanoic acid (1a), hexanoyl-CoA (2a), malonyl-CoA (3a), 3,5,7-trioxododecanoyl-CoA (4a), olivetol (5a), olivetolic acid (6a), geranyl pyrophosphate (7a), cannabigerolic acid (8a), cannabidiolic acid (9a), tetrahydrocannabinolic acid (10a), and cannabichromenic acid (11a). Hexanoic acid is an exemplary carboxylic acid substrate; other carboxylic acids may also be used (e.g., butyric acid, isovaleric acid, octanoic acid, decanoic acid, etc.; see e.g., FIG. 3 below). The enzymes that catalyze the synthesis of 3,5,7-trioxododecanoyl-CoA and olivetolic acid are shown in R2a and R3a, respectively, and can include multi-functional enzymes that catalyze the synthesis of 3,5,7-trioxododecanoyl-CoA and olivetolic acid. The enzymes cannabidiolic acid synthase (CBDAS), tetrahydrocannabinolic acid synthase (THCAS), and cannabichromenic acid synthase (CBCAS) that catalyze the synthesis of cannabidiolic acid, tetrahydrocannabinolic acid, and cannabichromenic acid, respectively, are shown in step R5a. FIG. 1 is adapted from Carvalho et al. “Designing Microorganisms for Heterologous Biosynthesis of Cannabinoids” (2017) FEMS Yeast Research June 1; 17(4), which is incorporated by reference in its entirety.

FIG. 2 is a schematic depicting a heterologous biosynthetic pathway for production of cannabinoid compounds, including five enzymatic steps mediated by: (R1) acyl activating enzymes (AAE); (R2) polyketide synthase enzymes (PKS) or bifunctional polyketide synthase-polyketide cyclase enzymes (PKS-PKC); (R3) polyketide cyclase enzymes (PKC) or bifunctional PKS-PKC enzymes; (R4) prenyltransferase enzymes (PT); and (R5) terminal synthase enzymes (TS). Any carboxylic acid of varying chain lengths, structures (e.g., aliphatic, alicyclic, or aromatic) and functionalization (e.g., hydroxylic-, keto-, amino-, thiol-, aryl-, or alogeno-) may also be used as precursor substrates (e.g., thiopropionic acid, hydroxy phenyl acetic acid, norleucine, bromodecanoic acid, butyric acid, isovaleric acid, octanoic acid, decanoic acid, etc).

FIG. 3 is a non-exclusive representation of select putative precursors for the cannabinoid pathway in FIG. 2.

FIG. 4 is a schematic showing a reaction catalyzed by a TS enzyme wherein the geranyl moiety of cannabigerolic acid (Formula (8a)) is cyclized to yield cannabidiolic acid, tetrahydrocannabinolic acid, or cannabichromenic acid.

FIG. 5 is a schematic showing a plasmid bearing the transcriptional unit encoding a TS. The coding sequence for the TS enzymes (labeled “Library gene”) was driven by the GAL1 promoter. Each TS enzyme possessed an N-terminally fused S. cerevisiae Mating Factor alpha 2 signal peptide (labeled “MFα2”) and a C-terminally fused HDEL signal peptide (labeled “HDEL”).

FIG. 6 depicts a graph showing secondary screening data for CBCA production based on an in vivo activity assay in S. cerevisiae. One library strain, strain t619896, expressing an Aspergillus niger (A. niger) CBCAS, including an N-terminally fused MFα2 signal peptide and a C-terminally fused HDEL signal peptide, was observed to produce CBCA. Strain t616313, expressing GFP, was used as a negative control. Strain t616315, expressing a C. sativa THCAS, including an N-terminally fused MFα2 signal peptide and a C-terminally fused HDEL signal peptide, was used as a positive control because it was observed to exhibit CBCAS activity as well as THCAS activity. The data represent the average of four biological replicates ±one standard deviation of the mean. Strain IDs and their corresponding activity from these graphs are shown in Table 5.

FIG. 7 depicts a graph showing production of CBCVA based on an in vivo activity assay in S. cerevisiae by library strain t619896. The data represent the average of four biological replicates ±one standard deviation of the mean. Strain IDs and their corresponding activity from these graphs are shown in Table 6.

FIGS. 8A-8C depict graphs showing secondary screening data of a library of TS variants for CBCA, THCA, and CBDA production based on an in vivo activity assay in S. cerevisiae. Strain t865843, expressing a C. sativa THCAS, including an N-terminally fused MFα2 signal peptide and a C-terminally fused HDEL signal peptide, was used as a positive control for THCAS activity. Strain t865768, expressing the A. niger CBCAS identified in Example 1, including an N-terminally fused MFα2 signal peptide and a C-terminally fused HDEL signal peptide, was used as a positive control for CBCAS activity. Strain t876607, expressing a C. sativa CBDAS, including an N-terminally fused MFα2 signal peptide and a C-terminally fused HDEL signal peptide, was used as a positive control for CBDAS activity. Strain t865842, expressing GFP, was used as a negative control. All library strains included an N-terminally fused MFα2 signal peptide and a C-terminally fused HDEL signal peptide. FIG. 8A depicts a graph showing CBCA production. FIG. 8B depicts a graph showing THCA production. FIG. 8C depicts a graph showing CBDA production. Strains depicted in FIGS. 8A-8C and their corresponding activity are shown in Table 8.

FIGS. 9A-9C depict graphs showing secondary screening data of a library of TS variants for cannabichromevarinic acid (CBCVA), tetrahydrocannabivarinic acid (THCVA), and cannabidivarinic acid (CBDVA) production based on an in vivo activity assay in S. cerevisiae. Strain t865843, expressing a C. sativa THCAS, including an N-terminally fused MFα2 signal peptide and a C-terminally fused HDEL signal peptide, was used as a positive control for THCVAS activity. Strain t865768, expressing the A. niger CBCAS identified in Example 1, including an N-terminally fused MFα2 signal peptide and a C-terminally fused HDEL signal peptide, was used as a positive control for CBCVAS activity. Strain t876607, expressing a C. sativa CBDAS, including an N-terminally fused MFα2 signal peptide and a C-terminally fused HDEL signal peptide, was used as a positive control for CBDVAS activity. Strain t865842, expressing GFP, was used as a negative control. All library strains included an N-terminally fused MFα2 signal peptide and a C-terminally fused HDEL signal peptide. FIG. 9A depicts a graph showing CBCVA production. FIG. 9B depicts a graph showing THCVA production. FIG. 9C depicts a graph showing CBDVA production. Strains depicted in FIGS. 9A-9C and their corresponding activity are shown in Table 9.

FIGS. 10A-10C depict graphs showing secondary screening activity data of candidate CBCAS enzymes identified in Example 3 for CBCA, THCA, and CBDA production based on an in vivo activity assay in S. cerevisiae. Strain t807925, expressing the A. niger CBCAS identified in Example 1, including an N-terminally fused MFα2 signal peptide and a C-terminally fused HDEL signal peptide, was used as a positive control for CBCAS activity. Strain t616313, expressing GFP, was used as a negative control. Strain t616314, expressing a Cannabis CBDAS, was used as a positive control for CBDAS activity. Strain t701870, expressing a Cannabis THCAS, was used as a positive control for THCAS activity. All library strains and positive control strains included an N-terminally fused MFα2 signal peptide and a C-terminally fused HDEL signal peptide. The data represent the average of four biological replicates ±one standard deviation of the mean. FIG. 10A depicts a graph showing CBCA production. FIG. 10B depicts a graph showing THCA production. FIG. 10C depicts a graph showing CBDA production. Strains depicted in FIGS. 10A-10C and their corresponding activity are shown in Table 10.

FIGS. 11A-11C depict graphs showing secondary screening activity data of candidate CBCAS enzymes identified in Example 3 for CBCVA, THCVA, and CBDVA production based on an in vivo activity assay in S. cerevisiae. Strain t807925, expressing the A. niger CBCAS identified in Example 1, including an N-terminally fused MFα2 signal peptide and a C-terminally fused HDEL signal peptide, was used as a positive control. Strain t616313, expressing GFP, was used as a negative control. Strain t616314, expressing a Cannabis CBDAS, was used as a positive control. Strain t701870, expressing a Cannabis THCAS, was used as a positive control. All library strains and positive control strains included an N-terminally fused MFα2 signal peptide and a C-terminally fused HDEL signal peptide. The data represent the average of four biological replicates ±one standard deviation of the mean. FIG. 11A depicts a graph showing CBCVA production. FIG. 11B depicts a graph showing THCVA production. FIG. 11C depicts a graph showing CBDVA production. Strains depicted in FIGS. 11A-11C and their corresponding activity are shown in Table 11.

FIGS. 12A-12B depict graphs showing substrate utilization of CBGA and CBGVA by candidate CBCAS enzymes identified in Example 3 based on an in vivo activity assay in S. cerevisiae. Strain t807925, expressing the A. niger CBCAS identified in Example 1, including an N-terminally fused MFα2 signal peptide and a C-terminally fused HDEL signal peptide, was used as a positive control. Strain t616313, expressing GFP, was used as a negative control. All library strains included an N-terminally fused MFα2 signal peptide and a C-terminally fused HDEL signal peptide. The data represent the average of four biological replicates ±one standard deviation of the mean. FIG. 12A depicts a graph showing CBGA substrate utilization. FIG. 12B depicts a graph showing CBGVA substrate utilization. Strains depicted in FIGS. 12A-12B and their corresponding activity are shown in Table 12.

FIG. 13 depicts a percent identity matrix of candidate CBCAS enzymes identified in Examples 3 and 4. The far-left column and the top row recite SEQ ID NOs corresponding to specific enzymes. SEQ ID NO: 27 corresponds to the protein sequence associated with UniProt Accession No. A0A254UC34 from A. niger. SEQ ID NO: 144 corresponds to the protein sequence associated with UniProt Accession No. A0A0C2SDS1, from Amanita muscaria; SEQ ID NO: 172 corresponds to the protein sequence associated with UniProt Accession No. B6HV04, from Penicillium rubens; SEQ ID NO: 166 corresponds to the protein sequence associated with UniProt Accession No. QOCYD9, from Aspergillus terreus; SEQ ID NO: 159 corresponds to the protein sequence associated with UniProt Accession No. A0A397IKU4, from Aspergillus turcosus; SEQ ID NO: 167 corresponds to the protein sequence associated with UniProt Accession No. A0A0K8LLN9, from Aspergillus udagawae; SEQ ID NO: 163 corresponds to the protein sequence associated with UniProt Accession NO. A0A2I1CBC7, from Aspergillus novofumigatus; SEQ ID NO: 165 corresponds to the protein sequence associated with UniProt Accession No. G3Y7J1, from Aspergillus niger; SEQ ID NO: 162 corresponds to the protein sequence associated with UniProt Accession No. A0A319AGI5, from Aspergillus lacticoffeatus; SEQ ID NO: 164 corresponds to the protein sequence associated with UniProt Accession No. A0A3F3PQ52, from Aspergillus welwitschiae; SEQ ID NO: 134 corresponds to the protein sequence associated with UniProt Accession No. A0A401KY63, from Aspergillus awamori; SEQ ID NO: 105 corresponds to the protein sequence associated with UniProt Accession No. A0A1L9NII2, from Aspergillus tubingensis; SEQ ID NO: 126 corresponds to the protein sequence associated with UniProt Accession No. A0A318Y6S9, from Aspergillus neoniger; SEQ ID NO: 155 corresponds to the protein sequence associated with UniProt Accession No. A0A319B6X5, from Aspergillus vadensis; SEQ ID NO: 112 corresponds to the protein sequence associated with UniProt Accession No. A0A0L1J4J1, from Aspergillus nomiae; and SEQ ID NO: 130 corresponds to the protein sequence associated with UniProt Accession No. Q2UF91, from Aspergillus oryzae. The value in each cell in the matrix is the percent identity between the amino acid sequences of the enzymes of the corresponding X and Y axes. Cells with 100% percent identity are shaded in black with white text and cells with 95-99.99% identity are shaded in grey.

FIG. 14 depicts a graph showing secondary screening activity data of candidate CBCAS enzymes identified in Example 3 for CBCA production based on an in vivo activity assay in S. cerevisiae. Strain 861555, expressing the A. niger CBCAS identified in Example 1 (referred to as “AnCBCAS”), including an N-terminally fused MFα2 signal peptide and a C-terminally fused HDEL signal peptide, was used as a positive control. Strain 861565 expresses the A. niger CBCAS identified in Example 1 (referred to as “AnCBCAS”) but excluding the N-terminally fused MFα2 signal peptide and the C-terminally fused HDEL signal peptide. All library strains were assayed in pairs with one strain including an N-terminally fused MFα2 signal peptide and a C-terminally fused HDEL signal peptide and the other strain excluding the N-terminally fused MFα2 signal peptide and C-terminally fused HDEL signal peptide. The data represent the average of four biological replicates ±one standard deviation of the mean. Strains depicted in FIG. 14 and their corresponding activity are shown in Table 13.

FIG. 15 is a ribbon diagram depicting the predicted location within the 3-dimensional structure of a Cannabis TS of sequence motifs that were identified as being enriched in candidate non-Cannabis CBCASs that were found to be effective in producing CBCA. Sequence motifs KVQARSGGH (SEQ ID NO: 174), CPTI[KR]TGGH (SEQ ID NO: 181), and P[IV]S[DQE]TTY[EDG]F[TA]DGLYDVLA[RQK]AVPES[VA]GHAYLGCPDP[RK]M (SEQ ID NO: 186), indicated by arrows, are predicted to contact the cofactor binding site.

FIG. 16 is a ribbon diagram depicting the predicted location within the 3-dimensional structure of a Cannabis TS of sequence motifs that were identified as being enriched in candidate non-Cannabis CBCASs that were found to be effective in producing CBCA. The active site of the TS is shown in dark gray. The FAD cofactor is shown as sticks at the right-hand side of the diagram. The triangular void shown in the middle of the figure is the substrate binding site. The motifs RT[EQ][PQ]APGLAVQYSY (SEQ ID NO: 207) and WQ[SA]FI[SA][AQ][KE]NLT[RW][QK]FY[NST]NM (SEQ ID NO: 211), indicated by arrows, are predicted to be near the substrate binding pocket.

### DETAILED DESCRIPTION

This disclosure provides methods for production of cannabinoids and cannabinoid precursors from fatty acid substrates using genetically modified host cells. Methods include heterologous expression of a terminal synthase (TS), such as a cannabichromenic acid synthase (CBCAS). The application describes TSs that can be functionally expressed in host cells such as S. cerevisiae. As demonstrated in the Examples, multiple non-Cannabis CBCASs were identified that were capable of producing cannabichromenic acid (CBCA) and cannabichromevarinic acid (CBCVA) in a host cell, as well as other TS products such as THCA, THCVA and CBDA. The TSs described in this disclosure may be useful in increasing the efficiency and purity of cannabinoid production such as, for example, by altering the activity and/or abundance of such enzymes.

### Definitions

While the following terms are believed to be well understood by one of ordinary skill in the art, the following definitions are set forth to facilitate explanation of the disclosed subject matter.

The term “a” or “an” refers to one or more of an entity, i.e., can identify a referent as plural. Thus, the terms “a” or “an,” “one or more” and “at least one” are used interchangeably in this application. In addition, reference to “an element” by the indefinite article “a” or “an” does not exclude the possibility that more than one of the elements is present, unless the context clearly requires that there is one and only one of the elements.

The terms “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure may refer to the “microorganisms” or “microbes” of lists/tables and figures present in the disclosure. This characterization can refer to not only the identified taxonomic genera of the tables and figures, but also the identified taxonomic species, as well as the various novel and newly identified or designed strains of any organism in the tables or figures. The same characterization holds true for the recitation of these terms in other parts of the specification, such as in the Examples.

The term “prokaryotes” is recognized in the art and refers to cells that contain no nucleus or other cell organelles. The prokaryotes are generally classified in one of two domains, the Bacteria and the Archaea.

“Bacteria” or “eubacteria” refers to a domain of prokaryotic organisms. Bacteria include at least 11 distinct groups as follows: (1) Gram-positive (gram+) bacteria, of which there are two major subdivisions: (a) high G+C group (Actinomycetes, Mycobacteria, Micrococcus, others) and (b) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; and (11) Thermotoga and Thermosipho thermophiles.

The term “Archaea” refers to a taxonomic classification of prokaryotic organisms with certain properties that make them distinct from Bacteria in physiology and phylogeny.

The term “Cannabis” refers to a genus in the family Cannabaceae. Cannabis is a dioecious plant. Glandular structures located on female flowers of Cannabis, called trichomes, accumulate relatively high amounts of a class of terpeno-phenolic compounds known as phytocannabinoids (described in further detail below). Cannabis has conventionally been cultivated for production of fibre and seed (commonly referred to as “hemp-type”), or for production of intoxicants (commonly referred to as “drug-type”). In drug-type Cannabis, the trichomes contain relatively high amounts of tetrahydrocannabinolic acid (THCA), which can convert to tetrahydrocannabinol (THC) via a decarboxylation reaction, for example upon combustion of dried Cannabis flowers, to provide an intoxicating effect. Drug-type Cannabis often contains other cannabinoids in lesser amounts. In contrast, hemp-type Cannabis contains relatively low concentrations of THCA, often less than 0.3% THC by dry weight. Hemp-type Cannabis may contain non-THC and non-THCA cannabinoids, such as cannabidiolic acid (CBDA), cannabidiol (CBD), and other cannabinoids. Presently, there is a lack of consensus regarding the taxonomic organization of the species within the genus. Unless context dictates otherwise, the term “Cannabis” is intended to include all putative species within the genus, such as, without limitation, Cannabis sativa, Cannabis indica, and Cannabis ruderalis and without regard to whether the Cannabis is hemp-type or drug-type.

The term “cyclase activity” in reference to a polyketide synthase (PKS) enzyme (e.g., an olivetol synthase (OLS) enzyme) or a polyketide cyclase (PKC) enzyme (e.g., an olivetolic acid cyclase (OAC) enzyme), refers to the activity of catalyzing the cyclization of an oxo fatty acyl-CoA (e.g., 3,5,7-trioxododecanoyl-COA, 3,5,7-trioxodecanoyl-COA) to the corresponding intramolecular cyclization product (e.g., olivetolic acid, divarinic acid). In some embodiments, the PKS or PKC catalyzes the C2-C7 aldol condensation of an acyl-COA with three additional ketide moieties added thereto.

A “cytosolic” or “soluble” enzyme refers to an enzyme that is predominantly localized (or predicted to be localized) in the cytosol of a host cell.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (i.e., bacteria and archaea) is that they have membrane-bound organelles, especially the nucleus, which contains the genetic material, and is enclosed by the nuclear envelope.

The term “host cell” refers to a cell that can be used to express a polynucleotide, such as a polynucleotide that encodes an enzyme used in biosynthesis of cannabinoids or cannabinoid precursors. The terms “genetically modified host cell,” “recombinant host cell,” and “recombinant strain” are used interchangeably and refer to host cells that have been genetically modified by, e.g., cloning and transformation methods, or by other methods known in the art (e.g., selective editing methods, such as CRISPR). Thus, the terms include a host cell (e.g., bacterial cell, yeast cell, fungal cell, insect cell, plant cell, mammalian cell, human cell, etc.) that has been genetically altered, modified, or engineered, so that it exhibits an altered, modified, or different genotype and/or phenotype, as compared to the naturally-occurring cell from which it was derived. It is understood that in some embodiments, the terms refer not only to the particular recombinant host cell in question, but also to the progeny or potential progeny of such a host cell.

The term “control host cell,” or the term “control” when used in relation to a host cell, refers to an appropriate comparator host cell for determining the effect of a genetic modification or experimental treatment. In some embodiments, the control host cell is a wild type cell. In other embodiments, a control host cell is genetically identical to the genetically modified host cell, except for the genetic modification(s) differentiating the genetically modified or experimental treatment host cell. In some embodiments, the control host cell has been genetically modified to express a wild type or otherwise known variant of an enzyme being tested for activity in other test host cells.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system, or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13(7): 563-567. A heterologous polynucleotide may comprise a wild-type sequence or a mutant sequence as compared with a reference polynucleotide sequence.

The term “at least a portion” or “at least a fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of an enzyme, such as a catalytic domain. A biologically active portion of a genetic regulatory element may comprise a portion or fragment of a full length genetic regulatory element and have the same type of activity as the full length genetic regulatory element, although the level of activity of the biologically active portion of the genetic regulatory element may vary compared to the level of activity of the full length genetic regulatory element.

A coding sequence and a regulatory sequence are said to be “operably joined” or “operably linked” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined if induction of a promoter in the 5′ regulatory sequence promotes transcription of the coding sequence and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

The terms “link,” “linked,” or “linkage” means two entities (e.g., two polynucleotides or two proteins) are bound to one another by any physicochemical means. Any linkage known to those of ordinary skill in the art, covalent or non-covalent, is embraced. In some embodiments, a nucleic acid sequence encoding an enzyme of the disclosure is linked to a nucleic acid encoding a signal peptide. In some embodiments, an enzyme of the disclosure is linked to a signal peptide. Linkage can be direct or indirect.

The terms “transformed” or “transform” with respect to a host cell refer to a host cell in which one or more nucleic acids have been introduced, for example on a plasmid or vector or by integration into the genome. In some instances where one or more nucleic acids are introduced into a host cell on a plasmid or vector, one or more of the nucleic acids, or fragments thereof, may be retained in the cell, such as by integration into the genome of the cell, while the plasmid or vector itself may be removed from the cell. In such instances, the host cell is considered to be transformed with the nucleic acids that were introduced into the cell regardless of whether the plasmid or vector is retained in the cell or not.

The term “volumetric productivity” or “production rate” refers to the amount of product formed per volume of medium per unit of time. Volumetric productivity can be reported in gram per liter per hour (g/L/h).

The term “specific productivity” of a product refers to the rate of formation of the product normalized by unit volume or mass or biomass and has the physical dimension of a quantity of substance per unit time per unit mass or volume [M·T⁻¹·M⁻¹ or M·T⁻¹·L⁻³, where M is mass or moles, T is time, L is length].

The term “biomass specific productivity” refers to the specific productivity in gram product per gram of cell dry weight (CDW) per hour (g/g CDW/h) or in mmol of product per gram of cell dry weight (CDW) per hour (mmol/g CDW/h). Using the relation of CDW to OD600 for the given microorganism, specific productivity can also be expressed as gram product per liter culture medium per optical density of the culture broth at 600 nm (OD) per hour (g/L/h/OD). Also, if the elemental composition of the biomass is known, biomass specific productivity can be expressed in mmol of product per C-mole (carbon mole) of biomass per hour (mmol/C-mol/h).

The term “yield” refers to the amount of product obtained per unit weight of a certain substrate and may be expressed as g product per g substrate (g/g) or moles of product per mole of substrate (mol/mol). Yield may also be expressed as a percentage of the theoretical yield. “Theoretical yield” is defined as the maximum amount of product that can be generated per a given amount of substrate as dictated by the stoichiometry of the metabolic pathway used to make the product and may be expressed as g product per g substrate (g/g) or moles of product per mole of substrate (mol/mol).

The term “titer” refers to the strength of a solution or the concentration of a substance in solution. For example, the titer of a product of interest (e.g., small molecule, peptide, synthetic compound, fuel, alcohol, etc.) in a fermentation broth is described as g of product of interest in solution per liter of fermentation broth or cell-free broth (g/L) or as g of product of interest in solution per kg of fermentation broth or cell-free broth (g/Kg).

The term “total titer” refers to the sum of all products of interest produced in a process, including but not limited to the products of interest in solution, the products of interest in gas phase if applicable, and any products of interest removed from the process and recovered relative to the initial volume in the process or the operating volume in the process. For example, the total titer of products of interest (e.g., small molecule, peptide, synthetic compound, fuel, alcohol, etc.) in a fermentation broth is described as g of products of interest in solution per liter of fermentation broth or cell-free broth (g/L) or as g of products of interest in solution per kg of fermentation broth or cell-free broth (g/Kg).

The term “amino acid” refers to organic compounds that comprise an amino group, —NH2, and a carboxyl group, —COOH. The term “amino acid” includes both naturally occurring and unnatural amino acids. Nomenclature for the twenty common amino acids is as follows: alanine (ala or A); arginine (arg or R); asparagine (asn or N); aspartic acid (asp or D); cysteine (cys or C); glutamine (gln or Q); glutamic acid (glu or E); glycine (gly or G); histidine (his or H); isoleucine (ile or I); leucine (leu or L); lysine (lys or K); methionine (met or M); phenylalanine (phe or F); proline (pro or P); serine (ser or S); threonine (thr or T); tryptophan (trp or W); tyrosine (tyr or Y); and valine (val or V). Non-limiting examples of unnatural amino acids include homo-amino acids, proline and pyruvic acid derivatives, 3-substituted alanine derivatives, glycine derivatives, ring-substituted phenylalanine derivatives, ring-substituted tyrosine derivatives, linear core amino acids, amino acids with protecting groups including Fmoc, Boc, and Cbz, j-amino acids (β3 and β2), and N-methyl amino acids.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of, or a substituent that is, a straight-chain or branched saturated hydrocarbon group having from 1 to 20 carbon atoms (“C1-20 alkyl”). In certain embodiments, the term “alkyl” refers to a radical of, or a substituent that is, a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 2 to 7 carbon atoms (“C2-7 alkyl”). In some embodiments, an alkyl group has 3 to 7 carbon atoms (“C3-7 alkyl”). In some embodiments, an alkyl group has 1 to 6 carbon atoms (“C₁₋₆ alkyl”). In some embodiments, an alkyl group has 2 to 6 carbon atoms (“C₂₋₆ alkyl”). In some embodiments, an alkyl group has 3 to 5 carbon atoms (“C₃₋₅ alkyl”). In some embodiments, an alkyl group has 5 carbon atoms (“C₅ alkyl”). In some embodiments, the alkyl group has 3 carbon atoms (“C3 alkyl”). In some embodiments, the alkyl group has 7 carbon atoms (“C7 alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”).

Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, benzyl).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described in this application that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

“Alkenyl” refers to a radical of, or a substituent that is, a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of, or a substituent that is, a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C), cyclohexenyl (C), cyclohexadienyl (C), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl. In certain embodiments, the aralkyl is 7-phenylheptanyl. In certain embodiments, the aralkyl is C7 alkyl substituted by an optionally substituted aryl group (e.g., phenyl). In certain embodiments, the aralkyl is a C7-C10 alkyl group substituted by an optionally substituted aryl group (e.g., phenyl).

“Partially unsaturated” refers to a group that includes at least one double or triple bond. A “partially unsaturated” ring system is further intended to encompass rings having multiple sites of unsaturation but is not intended to include aromatic groups (e.g., aryl or heteroaryl groups) as defined in this application. Likewise, “saturated” refers to a group that does not contain a double or triple bond, i.e., contains all single bonds.

The term “optionally substituted” means substituted or unsubstituted.

Alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described in this application that results in the formation of a stable compound. The present invention contemplates any and all such combinations in order to arrive at a stable compound. For purposes of this invention, heteroatoms such as nitrogen may have hydrogen substituents and/or any suitable substituent as described in this application which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃+X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃+X⁻, —P(OR^{cc})₃+X⁻, —P(R^{cc})₄, —P(OR^{cc})₄, —OP(R^{cc})₂, —OP(R^{cc})₃+X⁻, —OP(OR^{cc})₂, —OP(OR^{cc})₃+X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl;

wherein:

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃+X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃+X⁻, —NH(C₁₋₆ alkyl)₂+X⁻, —NH₂(C₁₋₆ alkyl)+X⁻, —NH₃+X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion. Alternatively, two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

wherein:

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc} —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two Rb groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃+X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃+X⁻, —NH(C₁₋₆ alkyl)₂+X⁻, —NH₂(C₁₋₆ alkyl)+X⁻, —NH₃+X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —CO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R⁹⁹ substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆, AsF₆⁻, SbF₆⁻, B[3,5-(CF₃)₂C₆H₃]₄]⁻, B(C₆F₅)₄⁻, BPh₄, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated by reference. Pharmaceutically acceptable salts of the compounds disclosed in this application include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of a compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (1), (9), (10), and (11) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R.0.5H₂O)), and polyhydrates (x is a number greater than 1, e.g., dihydrates (R.2 H₂O) and hexahydrates (R.6 H₂O)).

The term “tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, which are likewise formed by treatment with acid or base. Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and described by the R- and S-sequencing rules of Cahn and Prelog. An enantiomer can also be characterized by the manner in which the molecule rotates the plane of polarized light, and designated as dextrorotatory or levorotatory (i.e., as (+) or (−)-isomers respectively). A chiral compound can exist as either an individual enantiomer or as a mixture of enantiomers. A mixture containing equal proportions of the enantiomers is called a “racemic mixture.”

The term “co-crystal” refers to a crystalline structure comprising at least two different components (e.g., a compound described in this application and an acid), wherein each of the components is independently an atom, ion, or molecule. In certain embodiments, none of the components is a solvent. In certain embodiments, at least one of the components is a solvent. A co-crystal of a compound and an acid is different from a salt formed from a compound and the acid. In the salt, a compound described in this application is complexed with the acid in a way that proton transfer (e.g., a complete proton transfer) from the acid to a compound described in this application easily occurs at room temperature. In the co-crystal, however, a compound described in this application is complexed with the acid in a way that proton transfer from the acid to a compound described in this application does not easily occur at room temperature. In certain embodiments, in the co-crystal, there is no proton transfer from the acid to a compound described in this application. In certain embodiments, in the co-crystal, there is partial proton transfer from the acid to a compound described in this application. Co-crystals may be useful to improve the properties (e.g., solubility, stability, and ease of formulation) of a compound described in this application.

The term “polymorphs” refers to a crystalline form of a compound (or a salt, hydrate, or solvate thereof) in a particular crystal packing arrangement. All polymorphs of the same compound have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrug” refers to compounds, including derivatives of the compounds of Formula (X), (8), (9), (10), or (11), that have cleavable groups and become by solvolysis or under physiological conditions the compounds of Formula (X), (8), (9), (10), or (11) and that are pharmaceutically active in vivo. The prodrugs may have attributes such as, without limitation, solubility, bioavailability, tissue compatibility, or delayed release in a mammalian organism. Examples include, but are not limited to, derivatives of compounds described in this application, including derivatives formed from glycosylation of the compounds described in this application (e.g., glycoside derivatives), carrier-linked prodrugs (e.g., ester derivatives), bioprecursor prodrugs (a prodrug metabolized by molecular modification into the active compound), and the like. Non-limiting examples of glycoside derivatives are disclosed in and incorporated by reference from PCT Publication No. WO2018208875 and U.S. Patent Publication No. 2019/0078168. Non-limiting examples of ester derivatives are disclosed in and incorporated by reference from U.S. Patent Publication No. US2017/0362195.

Other derivatives of the compounds of this invention have activity in both their acid and acid derivative forms, but the acid sensitive form often offers advantages of solubility, bioavailability, tissue compatibility, or delayed release in a mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds of Formula (X), (8), (9), (10), or (11) may be preferred.

### Cannabinoids

As used in this application, the term “cannabinoid” includes compounds of Formula (X):

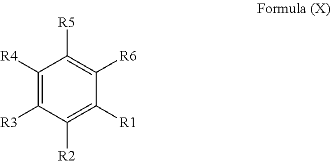

or a pharmaceutically acceptable salt, co-crystal, tautomer, stereoisomer, solvate, hydrate, polymorph, isotopically enriched derivative, or prodrug thereof, wherein R1 is hydrogen, optionally substituted acyl, optionally substituted alkyl, optionally substituted alkenyl, optionally substituted alkynyl, optionally substituted carbocyclyl, or optionally substituted aryl; R2 and R6 are, independently, hydrogen or carboxyl; R3 and R5 are, independently, hydroxyl, halogen, or alkoxy; and R4 is a hydrogen or an optionally substituted prenyl moiety; or optionally R4 and R3 are taken together with their intervening atoms to form a cyclic moiety, or optionally R4 and R5 are taken together with their intervening atoms to form a cyclic moiety, or optionally both 1) R4 and R3 are taken together with their intervening atoms to form a cyclic moiety and 2) R4 and R5 are taken together with their intervening atoms to form a cyclic moiety. In certain embodiments, R4 and R3 are taken together with their intervening atoms to form a cyclic moiety. In certain embodiments, R4 and R5 are taken together with their intervening atoms to form a cyclic moiety. In certain embodiments, “cannabinoid” refers to a compound of Formula (X), or a pharmaceutically acceptable salt thereof. In certain embodiments, both 1) R4 and R3 are taken together with their intervening atoms to form a cyclic moiety and 2) R4 and R5 are taken together with their intervening atoms to form a cyclic moiety.

In some embodiments, cannabinoids may be synthesized via the following steps: a) one or more reactions to incorporate three additional ketone moieties onto an acyl-CoA scaffold, where the acyl moiety in the acyl-CoA scaffold comprises between four and fourteen carbons; b) a reaction cyclizing the product of step (a); and c) a reaction to incorporate a prenyl moiety to the product of step (b) or a derivative of the product of step (b). In some embodiments, non-limiting examples of the acyl-CoA scaffold described in step (a) include hexanoyl-CoA and butyryl-CoA. In some embodiments, non-limiting examples of the product of step (b) or a derivative of the product of step (b) include olivetolic acid divarinic acid, and sphaerophorolic acid.

In some embodiments, a cannabinoid compound of Formula (X) is of Formula (X-A), (X-B), or (X-C):

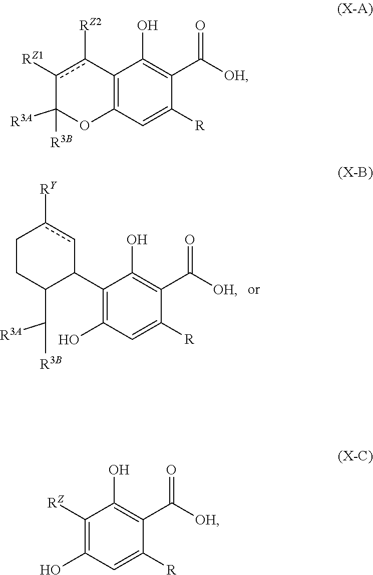

or a pharmaceutically acceptable salt, solvate, hydrate, polymorph, co-crystal, tautomer, stereoisomer, isotopically labeled derivative, or prodrug thereof;
wherein [symbol] is a double bond or a single bond, as valency permits;

R is hydrogen, optionally substituted acyl, optionally substituted alkyl, optionally substituted alkenyl, optionally substituted alkynyl, optionally substituted carbocyclyl, or optionally substituted aryl;

R^{Z1} is hydrogen, optionally substituted acyl, optionally substituted alkyl, optionally substituted alkenyl, optionally substituted alkynyl, optionally substituted carbocyclyl, or optionally substituted aryl;

R^{Z2} is hydrogen, optionally substituted acyl, optionally substituted alkyl, optionally substituted alkenyl, optionally substituted alkynyl, optionally substituted carbocyclyl, or optionally substituted aryl;

or optionally, R^{Z1} and R^{Z2} are taken together with their intervening atoms to form an optionally substituted carbocyclic ring;

R^{3A} is hydrogen, optionally substituted acyl, optionally substituted alkyl, optionally substituted alkenyl, or optionally substituted alkynyl;

R^{3B} is hydrogen, optionally substituted acyl, optionally substituted alkyl, optionally substituted alkenyl, or optionally substituted alkynyl;

R^{Y} is hydrogen, optionally substituted acyl, optionally substituted alkyl, optionally substituted alkenyl, or optionally substituted alkynyl;

R^{Z} is hydrogen, optionally substituted acyl, optionally substituted alkyl, optionally substituted alkenyl, or optionally substituted alkynyl.

In certain embodiments, a cannabinoid compound is of Formula (X-A):

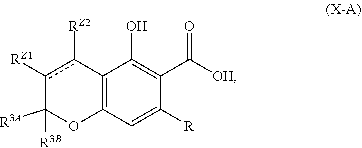

wherein [symbol] is a double bond, and each of R^{Z1} and R^{Z2} is hydrogen, one of R^{3A} and R^{3B} is optionally substituted C₂₋₆ alkenyl, and the other one of R^{3A} and R^{3B} is optionally substituted C₂₋₆ alkyl. In some embodiments, a cannabinoid compound of Formula (X) is of Formula (X-A), wherein each of R^{Z1} and R^{Z2} is hydrogen, one of R^{3A} and R^{3B} is a prenyl group, and the other one of R^{3A} and R^{3B} is optionally substituted methyl.

In certain embodiments, a cannabinoid compound of Formula (X) of Formula (X-A) is of Formula (11-z):

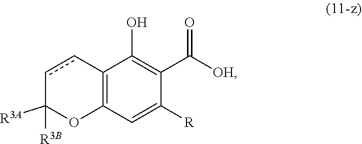

wherein [symbol] is a double bond or single bond, as valency permits; one of R^{3A} and R^{3B} is C₁₋₆ alkyl optionally substituted with alkenyl, and the other of R^{3A} and R^{3B} is optionally substituted C₁₋₆ alkyl. In certain embodiments, in a compound of Formula (11-z), [symbol] is a single bond; one of R^{3A} and R^{3B} is C₁₋₆ alkyl optionally substituted with prenyl; and the other of one of R^{3A} and R^{3B} is unsubstituted methyl; and R is as described in this application. In certain embodiments, in a compound of Formula (11-z), [symbol] is a single bond; one of R^{3A} and R^{3B} is

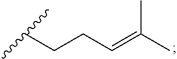 [SMILES: *CCC=C(C)C]

and the other of one of R^{3A} and R^{3B} is unsubstituted methyl; and R is as described in this application. In certain embodiments, a cannabinoid compound of Formula (11-z) is of Formula (11a):

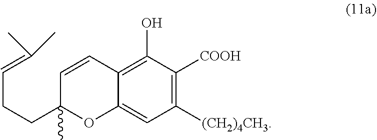

In certain embodiments, a cannabinoid compound of Formula (X) of Formula (X-A) is of Formula (11a):

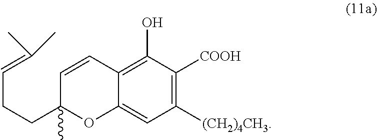

In certain embodiments, a cannabinoid compound of Formula (X-A) is of Formula (10-z):

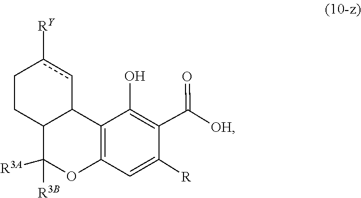

wherein [symbol] is a double bond or single bond, as valency permits; R^{Y} is hydrogen, optionally substituted acyl, optionally substituted alkyl, optionally substituted alkenyl, or optionally substituted alkynyl; and each of R^{3A} and R^{3B} is independently optionally substituted C₁₋₆ alkyl. In certain embodiments, in a compound of Formula (10-z), [symbol] is a single bond; each of R^{3A} and R^{3B} is unsubstituted methyl, and R is as described in this application. In certain embodiments, a cannabinoid compound of Formula (10-z) is of Formula (10a):

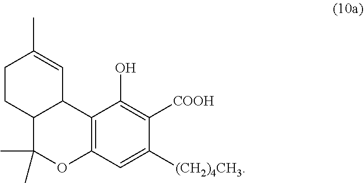

In certain embodiments, a compound of Formula (10a)

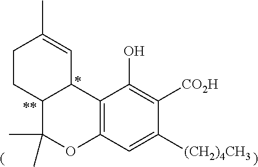

has a chiral atom labeled with * at carbon 10 and a chiral atom labeled with ** at carbon 6. In certain embodiments, in a compound of Formula (10a)

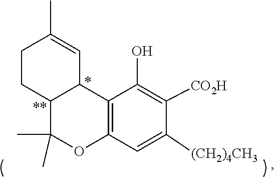

the chiral atom labeled with * at carbon 10 is of the R-configuration or S-configuration; and a chiral atom labeled with ** at carbon 6 is of the R-configuration. In certain embodiments, in a compound of Formula (10a)

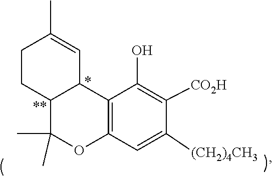

the chiral atom labeled with * at carbon 10 is of the S-configuration; and a chiral atom labeled with ** at carbon 6 is of the R-configuration or S-configuration. In certain embodiments, in a compound of Formula (10a)

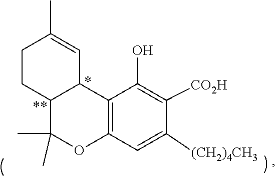

the chiral atom labeled with * at carbon 10 is of the R-configuration and a chiral atom labeled with ** at carbon 6 is of the R-configuration. In certain embodiments, a compound of Formula (10a)

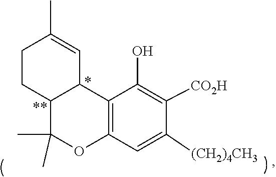

is of the formula:

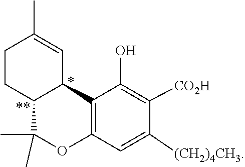

In certain embodiments, in a compound of Formula (10a)

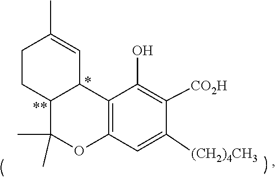

the chiral atom labeled with * at carbon 10 is of the S-configuration and a chiral atom labeled with ** at carbon 6 is of the S-configuration. In certain embodiments, a compound of Formula (10a)

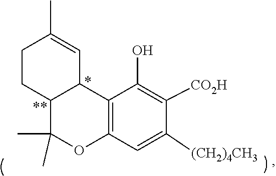

is of the formula:

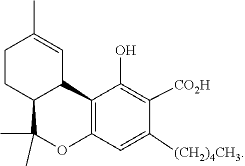

In certain embodiments, a cannabinoid compound is of Formula (X-B):

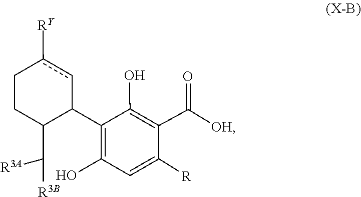

wherein [symbol] is a double bond; R^{Y} is hydrogen, optionally substituted acyl, optionally substituted alkyl, optionally substituted alkenyl, or optionally substituted alkynyl; and each of R^{3A} and R^{3B} is independently optionally substituted C₁₋₆ alkyl. In certain embodiments, in a compound of Formula (X-B), R^{Y} is optionally substituted C₁-6 alkyl; one of R^{3A} and R^{3B} is

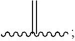

and the other one of R^{3A} and R^{3B} is unsubstituted methyl, and R is as described in this application. In certain embodiments, a compound of Formula (X-B) is of Formula (9a):

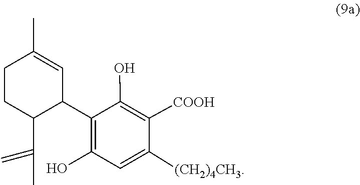

In certain embodiments, a compound of Formula (9a)

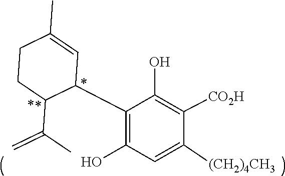

has a chiral atom labeled with * at carbon 3 and a chiral atom labeled with ** at carbon 4. In certain embodiments, in a compound of Formula (9a)

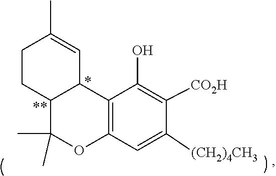

the chiral atom labeled with * at carbon 3 is of the R-configuration or S-configuration; and a chiral atom labeled with ** at carbon 4 is of the R-configuration. In certain embodiments, in a compound of Formula (9a)

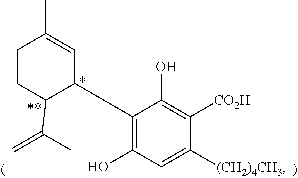

the chiral atom labeled with * at carbon 3 is of the S-configuration; and a chiral atom labeled with ** at carbon 4 is of the R-configuration or S-configuration. In certain embodiments, in a compound of Formula (9a)

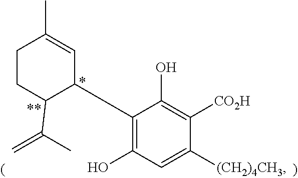

the chiral atom labeled with * at carbon 3 is of the R-configuration and a chiral atom labeled with ** at carbon 4 is of the R-configuration. In certain embodiments, a compound of Formula (9a)

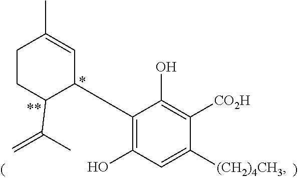

is of the formula:

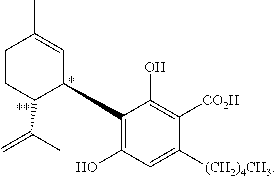

In certain embodiments, in a compound of Formula (9a)

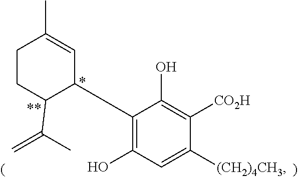

the chiral atom labeled with * at carbon 3 is of the S-configuration and a chiral atom labeled with ** at carbon 4 is of the S-configuration. In certain embodiments, a compound of Formula (9a)

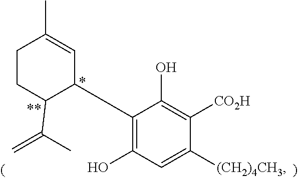

is of the formula:

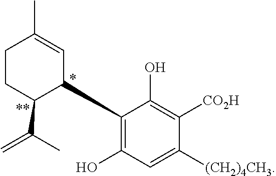

In certain embodiments, a cannabinoid compound is of Formula (X-C):

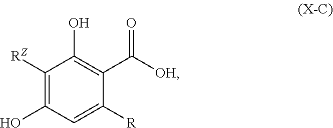

wherein R^{Z} is optionally substituted alkyl or optionally substituted alkenyl. In certain embodiments, a compound of Formula (X-C) is of formula:

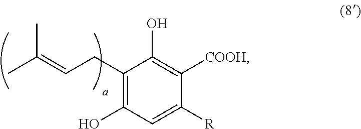

wherein a is 1, 2, 3, 4, 5, 6, 7, 8, 9, or 10. In certain embodiments, a is 1. In certain embodiments, a is 2. In certain embodiments, a is 3. In certain embodiments, a is 1, 2, or 3 for a compound of Formula (X-C). In certain embodiments, a cannabinoid compound is of Formula (X-C), and a is 1, 2, 3, 4, or 5. In certain embodiments, a compound of Formula (X-C) is of Formula (8a):

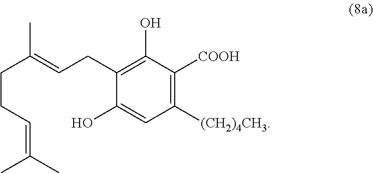

In some embodiments, cannabinoids of the present disclosure comprise cannabinoid receptor ligands. Cannabinoid receptors are a class of cell membrane receptors in the G protein-coupled receptor superfamily. Cannabinoid receptors include the CB₁ receptor and the CB₂ receptor. In some embodiments, cannabinoid receptors comprise GPR18, GPR55, and PPAR. (See Bram et al. “Activation of GPR18 by cannabinoid compounds: a tale of biased agonism” Br J Pharmcol v171 (16) (2014); Shi et al. “The novel cannabinoid receptor GPR55 mediates anxiolytic-like effects in the medial orbital cortex of mice with acute stress” Molecular Brain 10, No. 38 (2017); and O'Sullvan, Elizabeth. “An update on PPAR activation by cannabinoids” Br J Pharmcol v. 173(12) (2016)).

In some embodiments, cannabinoids comprise endocannabinoids, which are substances produced within the body, and phytocannabinoids, which are cannabinoids that are naturally produced by plants of genus Cannabis. In some embodiments, phytocannabinoids comprise the acidic and decarboxylated acid forms of the naturally-occurring plant-derived cannabinoids, and their synthetic and biosynthetic equivalents.

Over 94 phytocannabinoids have been identified to date (Berman, Paula, et al. “A new ESI-LC/MS approach for comprehensive metabolic profiling of phytocannabinoids in Cannabis.” Scientific reports 8.1 (2018): 14280; El-Alfy et al., 2010, “Antidepressant-like effect of delta-9-tetrahydrocannabinol and other cannabinoids isolated from Cannabis sativa L”, Pharmacology Biochemistry and Behavior 95 (4): 434-42; Rudolf Brenneisen, 2007, Chemistry and Analysis of Phytocannabinoids, Citti, Cinzia, et al. “A novel phytocannabinoid isolated from Cannabis sativa L. with an in vivo cannabimimetic activity higher than Δ9-tetrahydrocannabinol: Δ9-Tetrahydrocannabiphorol.” Sci Rep 9 (2019): 20335, each of which is incorporated by reference in this application in its entirety). In some embodiments, cannabinoids comprise Δ⁹-tetrahydrocannabinol (THC) type (e.g., (−)-trans-delta-9-tetrahydrocannabinol or dronabinol, (+)-trans-delta-9-tetrahydrocannabinol, (−)-cis-delta-9-tetrahydrocannabinol, or (+)-cis-delta-9-tetrahydrocannabinol), cannabidiol (CBD) type, cannabigerol (CBG) type, cannabichromene (CBC) type, cannabicyclol (CBL) type, cannabinodiol (CBND) type, or cannabitriol (CBT) type cannabinoids, or any combination thereof (see, e.g., R Pertwee, ed, Handbook of Cannabis (Oxford, UK: Oxford University Press, 2014)), which is incorporated by reference in this application in its entirety). A non-limiting list of cannabinoids comprises: cannabiorcol-C1 (CBNO), CBND-C1 (CBNDO), Δ⁹-trans-Tetrahydrocannabiorcolic acid-C1 (Δ⁹-THCO), Cannabidiorcol-C1 (CBDO), Cannabiorchromene-C1 (CBCO), (−)-Δ⁸-trans-(6aR,10aR)-Tetrahydrocannabiorcol-C1 (Δ⁸-THCO), Cannabiorcyclol C1 (CBLO), CBG-C1 (CBGO), Cannabinol-C2 (CBN-C2), CBND-C2, Δ⁹-THC-C2, CBD-C2, CBC-C2, Δ⁸-THC-C2, CBL-C2, Bisnor-cannabielsoin-C1 (CBEO), CBG-C2, Cannabivarin-C3 (CBNV), Cannabinodivarin-C3 (CBNDV), (−)-Δ⁹-trans-Tetrahydrocannabivarin-C3 (Δ⁹-THCV), (−)-Cannabidivarin-C3 (CBDV), (±)-Cannabichromevarin-C3 (CBCV), (−)-Δ⁸-trans-THC-C3 (Δ⁸-THCV), (±)-(1aS,3aR,8bR,8cR)-Cannabicyclovarin-C3 (CBLV), 2-Methyl-2-(4-methyl-2-pentenyl)-7-propyl-2H-1-benzopyran-5-ol, Δ⁷-tetrahydrocannabivarin-C3 (Δ⁷-THCV), CBE-C2, Cannabigerovarin-C3 (CBGV), Cannabitriol-C1 (CBTO), Cannabinol-C4 (CBN-C4), CBND-C4, (−)-Δ⁹-trans-Tetrahydrocannabinol-C4 (Δ⁹-THC-C4), Cannabidiol-C4 (CBD-C4), CBC-C4, (−)-trans-Δ⁸-THC-C4, CBL-C4, Cannabielsoin-C3 (CBEV), CBG-C4, CBT-C2, Cannabichromanone-C3, Cannabiglendol-C3 (OH-iso-HHCV-C3), Cannabioxepane-C5 (CBX), Dehydrocannabifuran-C5 (DCBF), Cannabinol-C5 (CBN), Cannabinodiol-C5 (CBND), (−)-Δ⁹-trans-Tetrahydrocannabinol-C5 (Δ⁹-THC), (−)-Δ⁸-trans-(6aR,10aR)-Tetrahydrocannabinol-C5 (Δ⁸-THC), (±)-Cannabichromene-C5 (CBC), (−)-Cannabidiol-C5 (CBD), (±)-(1aS,3aR,8bR,8cR)-CannabicyclolC5 (CBL), Cannabicitran-C5 (CBR), (−)-Δ⁹-(6aS,10aR-cis)-Tetrahydrocannabinol-C5 ((−)-cis-Δ⁹-THC), (−)-Δ⁷-trans-(1R,3R,6R)-Isotetrahydrocannabinol-C5 (trans-isoΔ⁷-THC), CBE-C4, Cannabigerol-C5 (CBG), Cannabitriol-C3 (CBTV), Cannabinol methyl ether-C5 (CBNM), CBNDM-C5, 8-OH—CBN-C5 (OH-CBN), OH-CBND-C5 (OH-CBND), 10-Oxo-Δ^{6a(10a)}-Tetrahydrocannabinol-C5 (OTHC), Cannabichromanone D-C5, Cannabicoumaronone-C5 (CBCON-C5), Cannabidiol monomethyl ether-C5 (CBDM), Δ⁹-THCM-C5, (±)-3″-hydroxy-Δ⁴″-cannabichromene-C5, (5aS,6S,9R,9aR)-Cannabielsoin-C5 (CBE), 2-geranyl-5-hydroxy-3-n-pentyl-1,4-benzoquinone-C5, 5-geranyl olivetolic acid. 5-geranyl olivetolate, 8α-Hydroxy-Δ⁹-Tetrahydrocannabinol-C5 (8α-OH-Δ⁹-THC), 8β-Hydroxy-Δ⁹-Tetrahydrocannabinol-C5 (8β-OH-Δ⁹-THC), 10α-Hydroxy-Δ⁸-Tetrahydrocannabinol-C5 (10α-OH-Δ⁸-THC), 10β-Hydroxy-Δ⁸-Tetrahydrocannabinol-C5 (10β-OH-Δ⁸-THC), 10α-hydroxy-Δ^{9,11}-hexahydrocannabinol-C5, 9β,10β-Epoxyhexahydrocannabinol-C5, OH-CBD-C5 (OH-CBD), Cannabigerol monomethyl ether-C5 (CBGM), Cannabichromanone-C5, CBT-C4, (±)-6,7-cis-epoxycannabigerol-C5, (±)-6,7-trans-epoxycannabigerol-C5, (−)-7-hydroxycannabichromane-C5, Cannabimovone-C5, (−)-trans-Cannabitriol-C5 ((−)-trans-CBT), (+)-trans-Cannabitriol-C5 ((+)-trans-CBT), (±)-cis-Cannabitriol-C5 ((±)-cis-CBT), (−)-trans-10-Ethoxy-9-hydroxy-Δ^{6a(10a)}-tetrahydrocannabivarin-C3 [(−)-trans-CBT-OEt], (−)-(6aR,9S,10S,10aR)-9,10-Dihydroxyhexahydrocannabinol-C5 [(−)-Cannabiripsol] (CBR), Cannabichromanone C-C5, (−)-6a,7,10a-Trihydroxy-Δ⁹-tetrahydrocannabinol-C5 [(−)-Cannabitetrol] (CBTT), Cannabichromanone B-C5, 8,9-Dihydroxy-Δ^{6a(10a)}-tetrahydrocannabinol-C5 (8,9-Di-OHCBT), (±)-4-acetoxycannabichromene-C5, 2-acetoxy-6-geranyl-3-n-pentyl-1,4-benzoquinone-C5, 11-Acetoxy-Δ9-TetrahydrocannabinolC5 (11-OAc-Δ9-THC), 5-acetyl-4-hydroxycannabigerol-C5, 4-acetoxy-2-geranyl-5-hydroxy-3-npentylphenol-C5, (−)-trans-10-Ethoxy-9-hydroxy-Δ^{6a(10a)}-tetrahydrocannabinol-C5 ((−)-trans-CBTOEt), sesquicannabigerol-C5 (SesquiCBG), carmagerol-C5, 4-terpenyl cannabinolate-C5, β-fenchyl-Δ⁹-tetrahydrocannabinolate-C5, α-fenchyl-Δ⁹-tetrahydrocannabinolate-C5, epi-bornyl-Δ⁹-tetrahydrocannabinolate-C5, bornyl-Δ⁹-tetrahydrocannabinolate-C5, α-terpenyl-Δ⁹-tetrahydrocannabinolate-C5, 4-terpenyl-Δ⁹-tetrahydrocannabinolate-C5, 6,6,9-trimethyl-3-pentyl-6H-dibenzo[b,d]pyran-1-ol, 3-(1,1-dimethylheptyl)-6,6a,7,8,10,10a-hexahydro-1-hydroxy-6,6-dimethyl-9H-dibenzo[1b,]pyran-9-one, (−)-(3S,4)-7-hydroxy-6-tetrahydrocannabinol-1,1-dimethylheptyl, (+)-(3S,4S)-7-hydroxy-Δ⁶-tetrahydrocannabinol-1,1-dimethylheptyl, 11-hydroxy-Δ⁹-tetrahydrocannabinol, and Δ⁸-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-methyl-4-phenylbutoxy]-1,9-phenanthridinediol 1-acetate)), certain aminoalkylindole analogs (e.g., (R)-(+)-[2,3-dihydro-5-methyl-3-(4-morpholinylmethyl)-pyrrolo[1,2,3-de]-1,4-benzoxazin-6-yl]-1-naphthalenyl-methanone), certain open pyran ring analogs (e.g., 2-[3-methy-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,2′,3′,4′,5′,6′-hexahydrobiphenyl, tetrahydrocannabiphorol (THCP), cannabidiphorol (CBDP), CBGP, CBCP, their acidic forms, salts of the acidic forms, dimers of any combination of the above, trimers of any combination of the above, polymers of any combination of the above, or any combination thereof.

A cannabinoid described in this application can be a rare cannabinoid. For example, in some embodiments, a cannabinoid described in this application corresponds to a cannabinoid that is naturally produced in conventional Cannabis varieties at concentrations of less than 10%, 9%, 8%, 7%, 6%, 5%, 4%, 3%, 2%, 1%, 0.9%, 0.8%, 0.7%, 0.6%, 0.5%, 0.25%, or 0.1% by dry weight of the female flower. In some embodiments, rare cannabinoids include CBGA, CBGVA, THCVA, CBDVA, CBCVA, and CBCA. In some embodiments, rare cannabinoids are cannabinoids that are not THCA, THC, CBDA or CBD.

A cannabinoid described in this application can also be a non-rare cannabinoid.

In some embodiments, the cannabinoid is selected from the cannabinoids listed in Table 1.

TABLE 1
Non-limiting examples of cannabinoids according to the present disclosure.

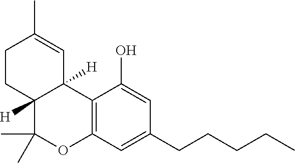 [SMILES: CCCCCc1cc(O)c2c(c1)OC(C)(C)[C@@H]1CCC(C)=C[C@@H]21]

Δ⁹-Tetrahydro-
cannabinol
Δ⁹-THC-C₅

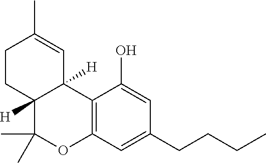 [SMILES: CCCCc1cc(O)c2c(c1)OC(C)(C)[C@@H]1CCC(C)=C[C@@H]21]

Δ⁹-Tetrahydro-
cannabinol-C₄
Δ⁹-THC-C₄

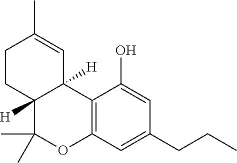 [SMILES: CCCc1cc(O)c2c(c1)OC(C)(C)[C@@H]1CCC(C)=C[C@@H]21]

Δ⁹-Tetrahydro-
cannabivarin
Δ⁹-THCV-C₃

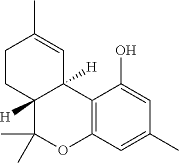

Δ⁹-Tetrahydro-
cannbiorcol
Δ⁹-THCO-C₁

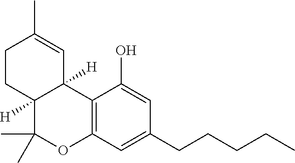 [SMILES: CCCCCc1cc(O)c2c(c1)OC(C)(C)[C@H]1CCC(C)=C[C@@H]21]

(−)-(6aS,10aR)-Δ⁹-
Tetrahydro-
cannabinol
(−)-cis-Δ⁹-THC-C₅

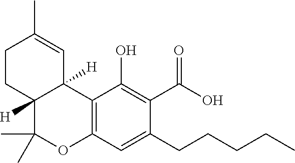 [SMILES: CCCCCc1cc2c(c(O)c1C(=O)O)[C@@H]1C=C(C)CC[C@H]1C(C)(C)O2]

Δ⁹-Tetrahydro-
cannabinolic acid A
Δ⁹-THCA-C₅ A

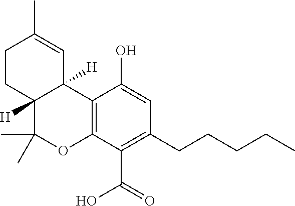 [SMILES: CCCCCc1cc(O)c2c(c1C(=O)O)OC(C)(C)[C@@H]1CCC(C)=C[C@@H]21]

Δ⁹-Tetrahydro-
cannabinolic acid B
Δ⁹-THCA-C₅ B

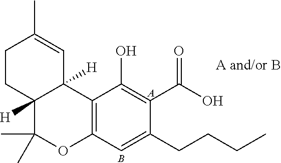

Δ⁹-Tetrahydro-
cannabinolic acid-C₄
A and/or B
Δ⁹-THCA-C₄ A and/or B

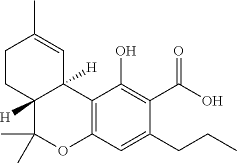 [SMILES: CCCc1cc2c(c(O)c1C(=O)O)[C@@H]1C=C(C)CC[C@H]1C(C)(C)O2]

Δ⁹-Tetrahydro-
cannabivarinic acid
A
Δ⁹-THCVA-C₃ A

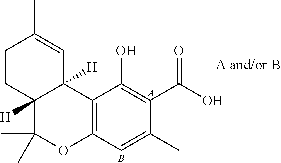

Δ⁹-Tetrahydro-
cannabiorcolic acid
A and/or B
Δ⁹-THCOA-C₁ A
and/or B

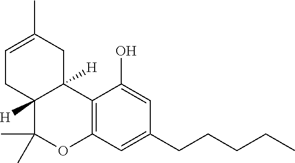 [SMILES: CCCCCc1cc(O)c2c(c1)OC(C)(C)[C@@H]1CC=C(C)C[C@@H]21]

(−)-Δ⁸-trans-
(6aR,10aR)-
Δ⁸-Tetrahydro-
cannabinol
Δ⁸-THC-C₅

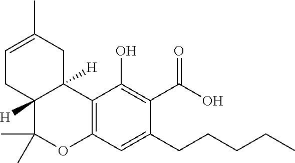 [SMILES: CCCCCc1cc2c(c(O)c1C(=O)O)[C@@H]1CC(C)=CC[C@H]1C(C)(C)O2]

(−)-Δ⁸-trans-
(6aR,10aR)-
Tetrahydro-
cannabinolic
acid A
Δ⁸-THCA-C₅ A

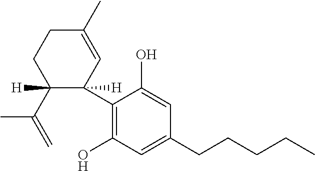 [SMILES: C=C(C)[C@@H]1CCC(C)=C[C@H]1c1c(O)cc(CCCCC)cc1O]

(−)-Cannabidiol
CBD-C5

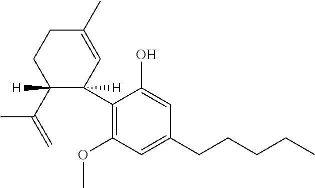 [SMILES: C=C(C)[C@@H]1CCC(C)=C[C@H]1c1c(O)cc(CCCCC)cc1OC]

Cannabidiol
momomethyl ether
CBDM-C5

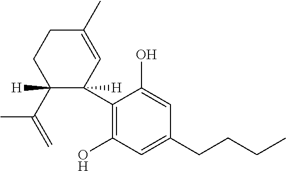 [SMILES: C=C(C)[C@@H]1CCC(C)=C[C@H]1c1c(O)cc(CCCC)cc1O]

Cannabidiol-C4
CBD-C4

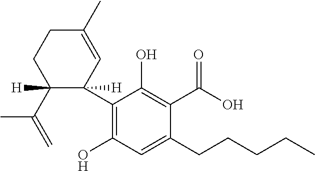 [SMILES: C=C(C)[C@@H]1CCC(C)=C[C@H]1c1c(O)cc(CCCCC)c(C(=O)O)c1O]

Cannabidiolic acid
CBDA-C5

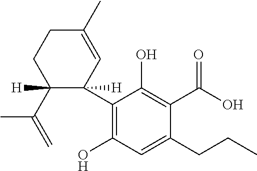 [SMILES: C=C(C)[C@@H]1CCC(C)=C[C@H]1c1c(O)cc(CCC)c(C(=O)O)c1O]

Cannabidivarinic acid
CBDVA-C3

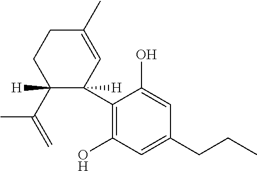 [SMILES: C=C(C)[C@@H]1CCC(C)=C[C@H]1c1c(O)cc(CCC)cc1O]

(−)-Cannabidivarin
CBDV-C3

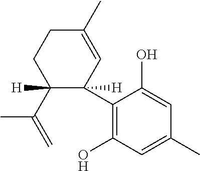 [SMILES: C=C(C)[C@@H]1CCC(C)=C[C@H]1c1c(O)cc(C)cc1O]

Cannabidiorcol
CBD-C1

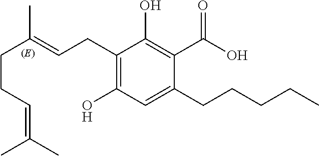 [SMILES: CCCCCc1cc(O)c(C/C=C(\C)CCC=C(C)C)c(O)c1C(=O)O]

Cannabigerolic acid
A
(E)-CBGA-C₅ A

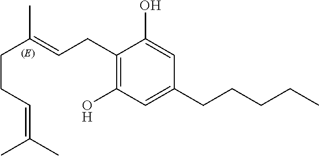 [SMILES: CCCCCc1cc(O)c(C/C=C(\C)CCC=C(C)C)c(O)c1]

Cannabigerol
(E)-CBG-C₅

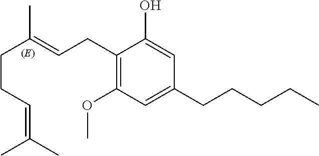 [SMILES: CCCCCc1cc(O)c(C/C=C(\C)CCC=C(C)C)c(OC)c1]

Cannabigerol
monomethyl ether
(E)-CBGM-C₅ A

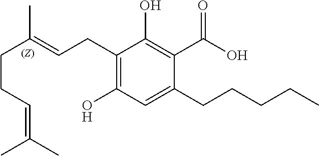 [SMILES: CCCCCc1cc(O)c(C/C=C(\C)CCC=C(C)C)c(O)c1C(=O)O]

Cannabinerolic acid A
(Z)-CBGA-C₅ A

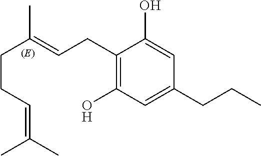 [SMILES: CCCc1cc(O)c(C/C=C(\C)CCC=C(C)C)c(O)c1]

Cannabigerovarin
(E)-CBGV-C₃

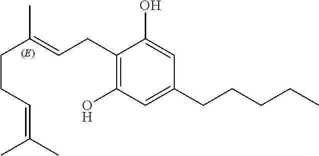 [SMILES: CCCCCc1cc(O)c(C/C=C(\C)CCC=C(C)C)c(O)c1]

Cannabigerol
(E)-CBG-C₅

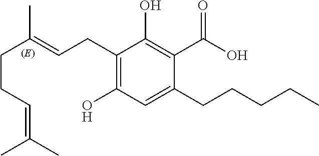 [SMILES: CCCCCc1cc(O)c(C/C=C(\C)CCC=C(C)C)c(O)c1C(=O)O]

Cannabigerolic acid
A
(E)-CBGA-C₅ A

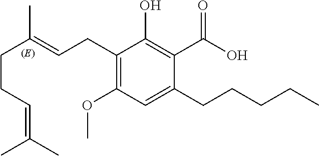 [SMILES: CCCCCc1cc(OC)c(C/C=C(\C)CCC=C(C)C)c(O)c1C(=O)O]

Cannabigerolic acid A
monomethyl ether
(E)-CBGAM-C₅ A

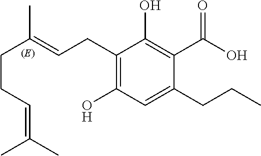 [SMILES: CCCc1cc(O)c(C/C=C(\C)CCC=C(C)C)c(O)c1C(=O)O]

Cannabigerovarinic
acid A
(E)-CBGVA-C₃ A

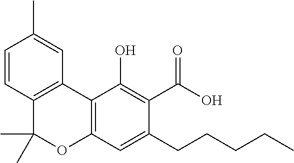 [SMILES: CCCCCc1cc2c(c(O)c1C(=O)O)-c1cc(C)ccc1C(C)(C)O2]

Cannabinolic acid A
CBNA-C5 A

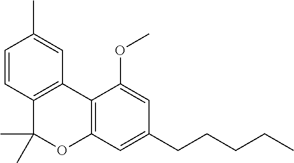 [SMILES: CCCCCc1cc(OC)c2c(c1)OC(C)(C)c1ccc(C)cc1-2]

Cannabinol methyl
ether
CBNM-C5

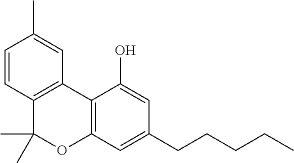 [SMILES: CCCCCc1cc(O)c2c(c1)OC(C)(C)c1ccc(C)cc1-2]

Cannabinol
CBN-C5

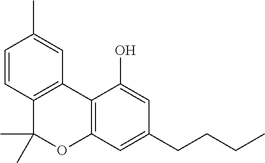 [SMILES: CCCCc1cc(O)c2c(c1)OC(C)(C)c1ccc(C)cc1-2]

Cannabinol-C4
CBN-C4

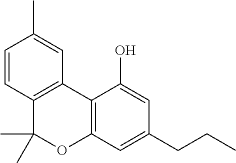 [SMILES: CCCc1cc(O)c2c(c1)OC(C)(C)c1ccc(C)cc1-2]

Cannabivarin
CBN-C3

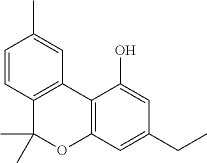 [SMILES: CCc1cc(O)c2c(c1)OC(C)(C)c1ccc(C)cc1-2]

Cannabinol-C2
CBN-C2

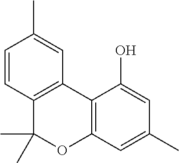

Cannabiorcol
CBN-C1

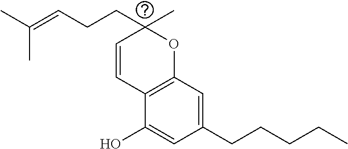 [SMILES: CCCCCc1cc(O)c2c(c1)OC(C)(CCC=C(C)C)C=C2]

(±)-
Cannabichromene
CBC-C₅

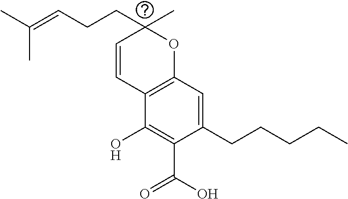 [SMILES: CCCCCc1cc2c(c(O)c1C(=O)O)C=CC(C)(CCC=C(C)C)O2]

(±)-Cannabichromenic
acid A
CBCA-C₅ A

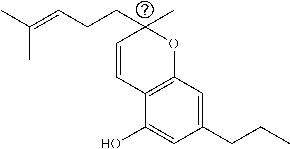 [SMILES: CCCc1cc(O)c2c(c1)OC(C)(CCC=C(C)C)C=C2]

(±)-
Cannabivarichromene,
(±)-
Cannabichromevarin
CBCV-C₃

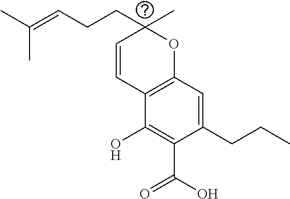 [SMILES: CCCc1cc2c(c(O)c1C(=O)O)C=CC(C)(CCC=C(C)C)O2]

(±)-Cannabichro-
mevarinic
acid A
CBCVA-C₃ A

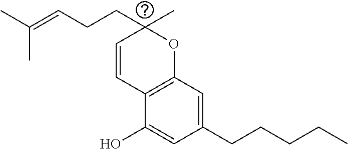 [SMILES: CCCCCc1cc(O)c2c(c1)OC(C)(CCC=C(C)C)C=C2]

(±)-
Cannabichromene
CBC-C₅

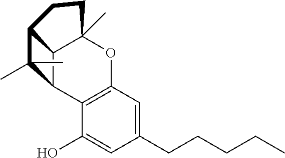

(±)-
(1aS,3aR,8bR,8cR)-
Cannabicyclol
CBL-C₅

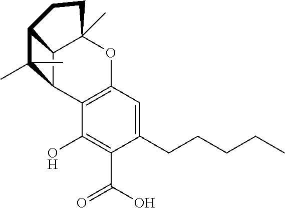

(±)-(1aS,3aR,8bR,8cR)-
Cannabicyclolic acid A
CBLA-C₅ A

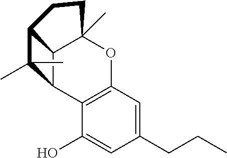

(±)-(1aS,3aR,8bR,8cR)-
cannabicyclovarin
CBLV-C₃

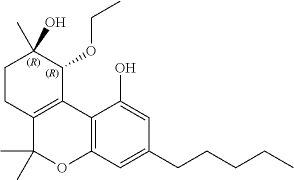

(−)-(9R,10R)-trans-
10-O-Ethyl-
cannabitriol
(−)-trans-CBT-OEt-
C5

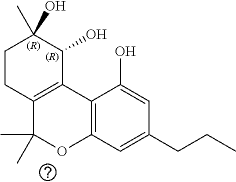

(±)-(9R,10R/9S,10S)-
Cannabitriol-C3
(±)-trans-CBT-C3

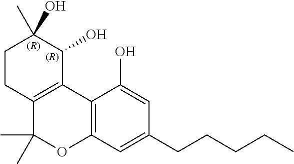

(−)-(9R,10R)-trans-
Cannabitriol
(−)-trans-CBT-C5

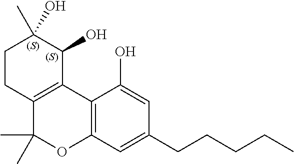

(+)-(9S,10S)-
Cannabitriol
(+)-trans-CBT-C5

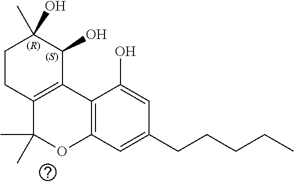

(±)-(9R,10S/9S,10R)-
Cannabitriol
(±)-cis-CBT-C5

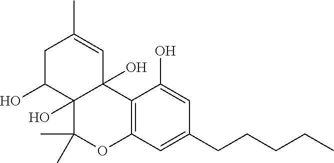 [SMILES: CCCCCc1cc(O)c2c(c1)OC(C)(C)C1(O)C(O)CC(C)=CC21O]

(−)-6a,7,10a-
Trihydroxy-
Δ9-
tetrahydrocannabinol
(−)-Cannabitetrol

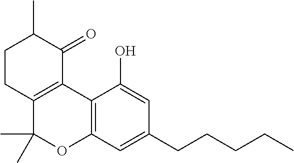 [SMILES: CCCCCc1cc(O)c2c(c1)OC(C)(C)C1=C2C(=O)C(C)CC1]

10-Oxo-Δ6a(10a)-
tetrahydro-
cannabinol
OTHC

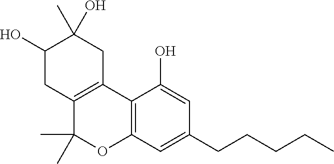 [SMILES: CCCCCc1cc(O)c2c(c1)OC(C)(C)C1=C2CC(C)(O)C(O)C1]

8,9-Dihydroxy-
Δ6a(10a)-
tetrahydro-
cannabinol
8,9-Di-OH-CBT-C5

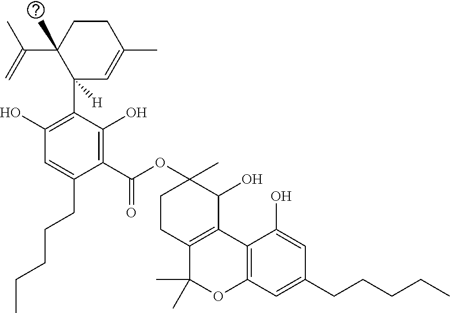 [SMILES: C=C(C)[C@]1(C)CCC(C)=C[C@H]1c1c(O)cc(CCCCC)c(C(=O)OC2(C)CCC3=C(c4c(O)cc(CCCCC)cc4OC3(C)C)C2O)c1O]

Cannabidiolic acid A
cannabitriol ester
CBDA-C5 9-OH-CBT-C5
ester

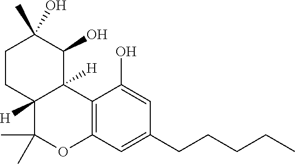 [SMILES: CCCCCc1cc(O)c2c(c1)OC(C)(C)[C@@H]1CC[C@](C)(O)[C@@H](O)[C@@H]21]

(−)-(6aR,9S,10S,10aR)-
9,10-Dihydroxy-
hexahydrocannabinol,
Cannabiripsol
Cannabiripsol-C5

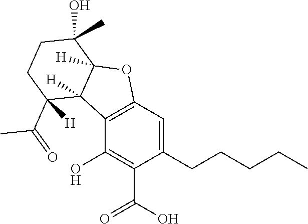

(5aS,6S,9R,9aR)-
Cannabielsoic acid B
CBEA-C5 B

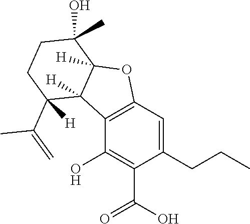

(5aS,6S,9R,9aR)-
C3-Cannabielsoic
acid B
CBEA-C3 B

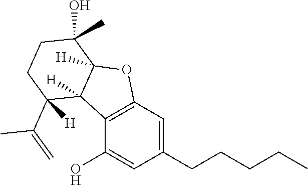

(5aS,6S,9R,9aR)-
Cannabielsoin
CBE-C5

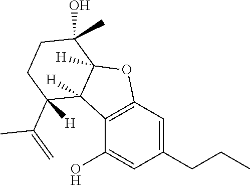

(5aS,6S,9R,9aR)-
C3-Cannabielsoin
CBE-C3

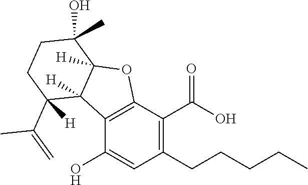

(5aS,6S,9R,9aR)-
Cannabielsoic acid A
CBEA-C5 A

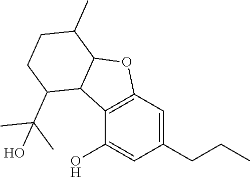 [SMILES: CCCc1cc(O)c2c(c1)OC1C(C)CCC(C(C)(C)O)C21]

Cannabiglendol-C3
OH-iso-HHCV-C3

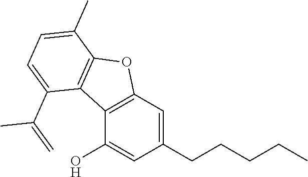 [SMILES: C=C(C)c1ccc(C)c2oc3cc(CCCCC)cc(O)c3c12]

Dehydro-
cannabifuran
DCBF-C5

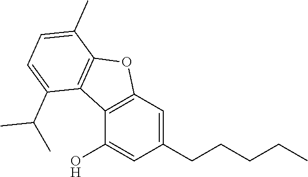 [SMILES: CCCCCc1cc(O)c2c(c1)oc1c(C)ccc(C(C)C)c12]

Cannabifuran
CBF-C5

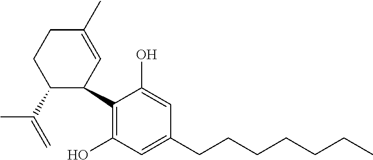 [SMILES: C=C(C)[C@@H]1CCC(C)=C[C@H]1c1c(O)cc(CCCCCCC)cc1O]

Cannabidiphorol
(CBDP)

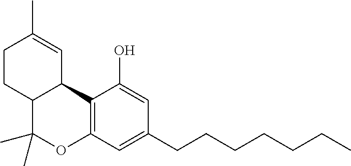

Tetrahydro-
cannabiphorol
(THCP)

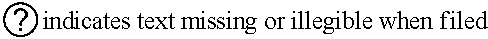

Cannabinoids are often classified by “type,” i.e., by the topological arrangement of their prenyl moieties (See, for example, M. A. Elsohly and D. Slade, Life Sci., 2005, 78, 539-548; and L. O. Hanus et al. Nat. Prod. Rep., 2016, 33, 1357). Generally, each “type” of cannabinoid includes the variations possible for ring substitutions of the resorcinol moiety at the position meta to the two hydroxyl moieties. As used herein, a “CBG-type” cannabinoid is a 3-[(2E)-3,7-dimethylocta-2,6-dienyl]-2,4-dihydroxybenzoic acid optionally substituted at the 6 position of the benzoic acid moiety. As used herein, “CBC-type” cannabinoids refer to 5-hydroxy-2-methyl-2-(4-methylpent-3-enyl)-chromene-6-carboxylic acid optionally substituted at the 7 position of the chromene moiety. As used herein, a “THC-type” cannabinoid is a (6aR,10aR)-1-hydroxy-6,6,9-trimethyl-6a,7,8,10a-tetrahydrobenzo[c]chromene-2-carboxylic acid optionally substituted at the 3 position of the benzo[c]chromene moiety. As used herein, a “CBD-type” cannabinoid is a 2,4-dihydroxy-3-[(1R,6R)-3-methyl-6-prop-1-en-2-ylcyclohex-2-en-1-yl]-benzoic acid optionally substituted at the 6 position of the benzoic acid moiety. In some embodiments, the optional ring substitution for each “type” is an optionally substituted C1-C11 alkyl, an optionally substituted C1-C11 alkenyl, an optionally substituted C1-C11 alkynyl, or an optionally substituted C1-C11 aralkyl.

### Biosynthesis of Cannabinoids and Cannabinoid Precursors

Aspects of the present disclosure provide tools, sequences, and methods for the biosynthetic production of cannabinoids in host cells. In some embodiments, the present disclosure teaches expression of enzymes that are capable of producing cannabinoids by biosynthesis.

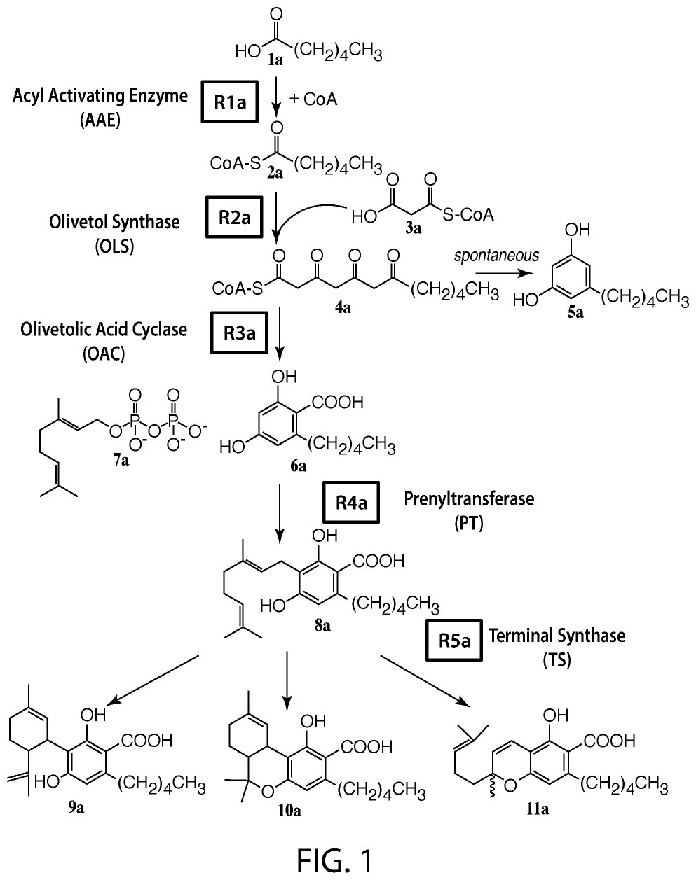

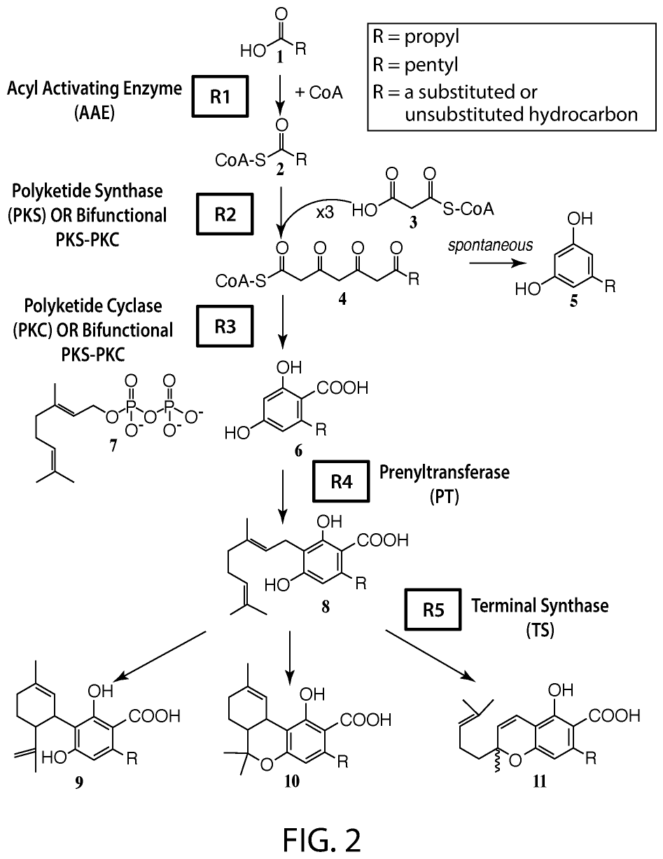

As a non-limiting example, one or more of the enzymes depicted in FIG. 2 may be used to produce a cannabinoid or cannabinoid precursor of interest. FIG. 1 shows a cannabinoid biosynthesis pathway for the most abundant phytocannabinoids found in Cannabis. See also, de Meijer et al. I, II, III, and IV (I: 2003, Genetics, 163:335-346; II: 2005, Euphytica, 145:189-198; III: 2009, Euphytica, 165:293-311; and IV: 2009, Euphytica, 168:95-112), and Carvalho et al. “Designing Microorganisms for Heterologous Biosynthesis of Cannabinoids” (2017) FEMS Yeast Research June 1; 17(4), each of which is incorporated by reference in this application in its entirety.

It should be appreciated that a precursor substrate for use in cannabinoid biosynthesis is generally selected based on the cannabinoid of interest. Non-limiting examples of cannabinoid precursors include compounds of Formulae (1)-(8) in FIG. 2. In some embodiments, polyketides, including compounds of Formula (5), could be prenylated. In certain embodiments, the precursor is a precursor compound shown in FIG. 1, 2, or 3. Substrates in which R contains 1-40 carbon atoms are preferred. In some embodiments, substrates in which R contains 3-8 carbon atoms are most preferred.

As used in this application, a cannabinoid or a cannabinoid precursor may comprise an R group. See, e.g., FIG. 2. In some embodiments, R may be a hydrogen. In certain embodiments, R is optionally substituted alkyl. In certain embodiments, R is optionally substituted C1-40 alkyl. In certain embodiments, R is optionally substituted C2-40 alkyl. In certain embodiments, R is optionally substituted C2-40 alkyl, which is straight chain or branched alkyl. In certain embodiments, R is optionally substituted C3-8 alkyl. In certain embodiments, R is optionally substituted C1-C40 alkyl, C1-C20 alkyl, C1-C10 alkyl, C1-C8 alkyl, C1-C5 alkyl, C3-C5 alkyl, C3 alkyl, or C5 alkyl. In certain embodiments, R is optionally substituted C1-C20 alkyl. In certain embodiments, R is optionally substituted C1-C10 alkyl. In certain embodiments, R is optionally substituted C1-C8 alkyl. In certain embodiments, R is optionally substituted C1-C5 alkyl. In certain embodiments, R is optionally substituted C1-C7 alkyl. In certain embodiments, R is optionally substituted C3-C5 alkyl. In certain embodiments, R is optionally substituted C3 alkyl. In certain embodiments, R is unsubstituted C3 alkyl. In certain embodiments, R is n-C3 alkyl. In certain embodiments, R is n-propyl. In certain embodiments, R is n-butyl. In certain embodiments, R is n-pentyl. In certain embodiments, R is n-hexyl. In certain embodiments, R is n-heptyl. In certain embodiments, R is of formula:

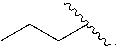 [SMILES: *CCC]

In certain embodiments, R is optionally substituted C4 alkyl. In certain embodiments, R is unsubstituted C4 alkyl. In certain embodiments, R is optionally substituted C5 alkyl. In certain embodiments, R is unsubstituted C5 alkyl. In certain embodiments, R is optionally substituted C6 alkyl. In certain embodiments, R is unsubstituted C6 alkyl. In certain embodiments, R is optionally substituted C7 alkyl. In certain embodiments, R is unsubstituted C7 alkyl. In certain embodiments, R is of formula:

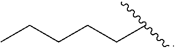 [SMILES: *CCCCC]

In certain embodiments, R is of formula:

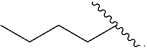 [SMILES: *CCCC]

In certain embodiments, R is of formula:

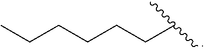 [SMILES: *CCCCCC]

In certain embodiments, R is of formula:

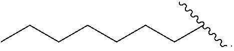 [SMILES: *CCCCCCC]

In certain embodiments, R is of formula:

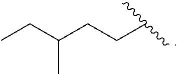 [SMILES: *CCC(C)CC]

In certain embodiments, R is optionally substituted n-propyl. In certain embodiments, R is n-propyl optionally substituted with optionally substituted aryl. In certain embodiments, R is n-propyl optionally substituted with optionally substituted phenyl. In certain embodiments, R is n-propyl substituted with unsubstituted phenyl. In certain embodiments, R is optionally substituted butyl. In certain embodiments, R is optionally substituted n-butyl. In certain embodiments, R is n-butyl optionally substituted with optionally substituted aryl. In certain embodiments, R is n-butyl optionally substituted with optionally substituted phenyl. In certain embodiments, R is n-butyl substituted with unsubstituted phenyl. In certain embodiments, R is optionally substituted pentyl. In certain embodiments, R is optionally substituted n-pentyl. In certain embodiments, R is n-pentyl optionally substituted with optionally substituted aryl. In certain embodiments, R is n-pentyl optionally substituted with optionally substituted phenyl. In certain embodiments, R is n-pentyl substituted with unsubstituted phenyl. In certain embodiments, R is optionally substituted hexyl. In certain embodiments, R is optionally substituted n-hexyl. In certain embodiments, R is optionally substituted n-heptyl. In certain embodiments, R is optionally substituted n-octyl. In certain embodiments, R is alkyl optionally substituted with aryl (e.g., phenyl). In certain embodiments, R is optionally substituted acyl (e.g., —C(═O)Me).

In certain embodiments, R is optionally substituted alkenyl (e.g., substituted or unsubstituted C₂₋₆ alkenyl). In certain embodiments, R is substituted or unsubstituted C₂₋₆ alkenyl. In certain embodiments, R is substituted or unsubstituted C₂₋₅ alkenyl. In certain embodiments, R is of formula:

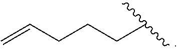 [SMILES: *CCCC=C]

In certain embodiments, R is optionally substituted alkynyl (e.g., substituted or unsubstituted C₂₋₆ alkynyl). In certain embodiments, R is substituted or unsubstituted C₂₋₆ alkynyl. In certain embodiments, R is of formula:

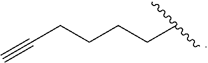 [SMILES: *CCCCC#C]

In certain embodiments, R is optionally substituted carbocyclyl. In certain embodiments, R is optionally substituted aryl (e.g., phenyl or napthyl).

The chain length of a precursor substrate can be from C1-C40. Those substrates can have any degree and any kind of branching or saturation or chain structure, including, without limitation, aliphatic, alicyclic, and aromatic. In addition, they may include any functional groups including hydroxy, halogens, carbohydrates, phosphates, methyl-containing or nitrogen-containing functional groups.

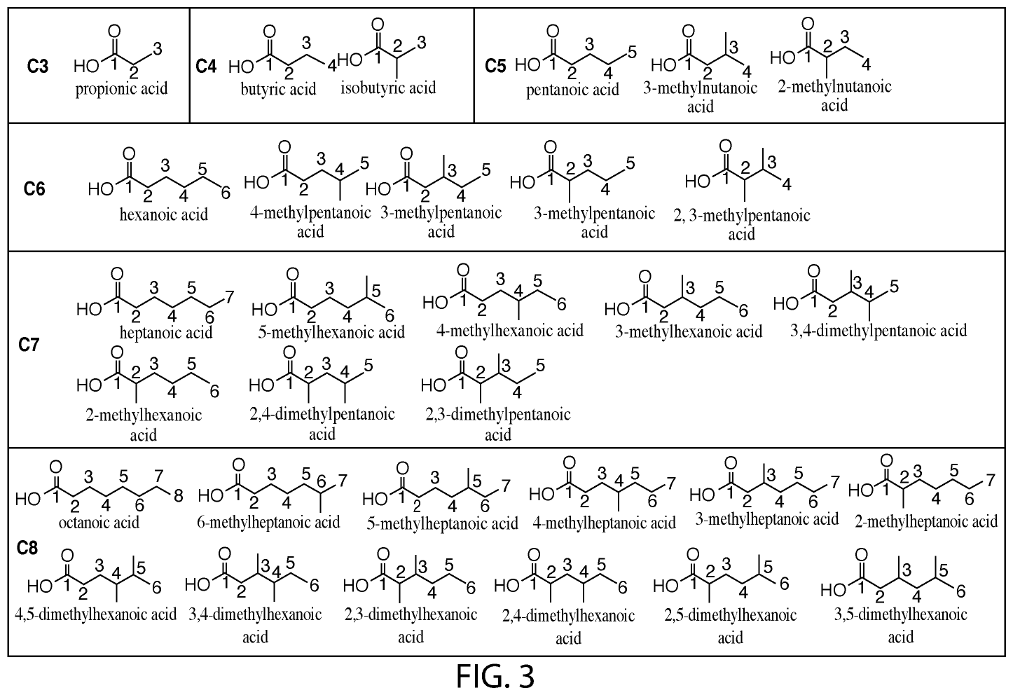

For example, FIG. 3 shows a non-exclusive set of putative precursors for the cannabinoid pathway. Aliphatic carboxylic acids including four to eight total carbons (“C4”-“C8” in FIG. 3) and up to 10-12 total carbons with either linear or branched chains may be used as precursors for the heterologous pathway. Non-limiting examples include methanoic acid, butyric acid, pentanoic acid, hexanoic acid, heptanoic acid, isovaleric acid, octanoic acid, and decanoic acid. Additional precursors may include ethanoic acid and propanoic acid. In some embodiments, in addition to acids, the ester, salt, and acid forms may all be used as substrates. Substrates may have any degree and any kind of branching, saturation, and chain structure, including, without limitation, aliphatic, alicyclic, and aromatic. In addition, they may include any functional modifications or combination of modifications including, without limitation, halogenation, hydroxylation, amination, acylation, alkylation, phenylation, and/or installation of pendant carbohydrates, phosphates, sulfates, heterocycles, or lipids, or any other functional groups.

Substrates for any of the enzymes disclosed in this application may be provided exogenously or may be produced endogenously by a host cell. In some embodiments, the cannabinoids are produced from a glucose substrate, so that compounds of Formula 1 shown in FIG. 2 and CoA precursors are synthesized by the cell. In other embodiments, a precursor is fed into the reaction. In some embodiments, a precursor is a compound selected from Formulae 1-8 in FIG. 2.

Cannabinoids produced by methods disclosed in this application include rare cannabinoids. Due to the low concentrations at which cannabinoids, including rare cannabinoids occur in nature, producing industrially significant amounts of isolated or purified cannabinoids from the Cannabis plant may become prohibitive due to, e.g., the large volumes of Cannabis plants, and the large amounts of space, labor, time, and capital requirements to grow, harvest, and/or process the plant materials (see, for example, Crandall, K., 2016. A Chronic Problem: Taming Energy Costs and Impacts from Marijuana Cultivation. EQ Research; Mills, E., 2012. The carbon footprint of indoor Cannabis production. Energy Policy, 46, pp. 58-67; Jourabchi, M. and M. Lahet. 2014. Electrical Load Impacts of Indoor Commercial Cannabis Production. Presented to the Northwest Power and Conservation Council; O'Hare, M., D. Sanchez, and P. Alstone. 2013. Environmental Risks and Opportunities in Cannabis Cultivation. Washington State Liquor and Cannabis Board; 2018. Comparing Cannabis Cultivation Energy Consumption. New Frontier Data; and Madhusoodanan, J., 2019. Can cannabis go green? Nature Outlook: Cannabis; all of which are incorporated by reference in this disclosure). The disclosure provided in this application represents a potentially efficient method for producing high yields of cannabinoids, including rare cannabinoids. The disclosure provided in this application also represents a potential method for addressing concerns related to agricultural practices and water usage associated with traditional methods of cannabinoid production (Dillis et al. “Water storage and irrigation practices for cannabis drive seasonal patterns of water extraction and use in Northern California.” Journal of Environmental Management 272 (2020): 110955, incorporated by reference in this disclosure).

Cannabinoids produced by the disclosed methods also include non-rare cannabinoids. Without being bound by a particular theory, the methods described in this application may be advantageous compared with traditional plant-based methods for producing non-rare cannabinoids. For example, methods provided in this application represent potentially efficient means for producing consistent and high yields of non-rare cannabinoids. With traditional methods of cannabinoid production, in which cannabinoids are harvested from plants, maintaining consistent and uniform conditions, including airflow, nutrients, lighting, temperature, and humidity, can be difficult. For example, with plant-based methods, there can be microclimates created by branching, which can lead to inconsistent yields and by-product formation. In some embodiments, the methods described in this application are more efficient at producing a cannabinoid of interest as compared to harvesting cannabinoids from plants. For example, with plant-based methods, seed-to-harvest can take up to half a year, while cutting-to-harvest usually takes about 4 months. Additional steps including drying, curing, and extraction are also usually needed with plant-based methods. In contrast, in some embodiments, the fermentation-based methods described in this application only take about 1, 2, 3, 4, 5, 6, 7, 8, 9, or 10 days. In some embodiments, the fermentation-based methods described in this application only take about 3-5 days. In some embodiments, the fermentation-based methods described in this application only take about 5 days. In some embodiments, the methods provided in this application reduce the amount of security needed to comply with regulatory standards. For example, a smaller secured area may be needed to be monitored and secured to practice the methods described in this application as compared to the cultivation of plants. In some embodiments, the methods described in this application are advantageous over plant-sourced cannabinoids.

### Terminal Synthases (TS)

A host cell described in this application may comprise a terminal synthase (TS). As used in this application, a “TS” refers to an enzyme that is capable of catalyzing oxidative cyclization of a prenyl moiety (e.g., terpene) to produce a ring-containing product (e.g., heterocyclic ring-containing product). In certain embodiments, a TS is capable of catalyzing oxidative cyclization of a prenyl moiety (e.g., terpene) to produce a carbocyclic-ring containing product (e.g., cannabinoid). In certain embodiments, a TS is capable of catalyzing oxidative cyclization of a prenyl moiety (e.g., terpene) to produce a heterocyclic-ring containing product (e.g., cannabinoid). In certain embodiments, a TS is capable of catalyzing oxidative cyclization of a prenyl moiety (e.g., terpene) to produce a cannabinoid.

TS enzymes are monomers that include FAD-binding and Berberine Bridge Enzyme (BBE) sequence motifs.

In some embodiments, the TS is an “ancestral” terminal synthase. Ancestral TSes can be generated from probabilistic models of mutations applied to terminal synthase phylogenes based on transcriptomic datasets. For example, Hochberg et al., describe a process for reconstructing ancestral proteins in Annu. Rev. Biophys. 2017. 46:247-69, which is incorporated by reference in its entirety in this disclosure.

a. Substrates

A TS may be capable of using one or more substrates. In some instances, the location of the prenyl group and/or the R group differs between TS substrates. For example, a TS may be capable of using as a substrate one or more compounds of Formula (8w), Formula (8x), Formula (8′), Formula (8y), and/or Formula (8z):

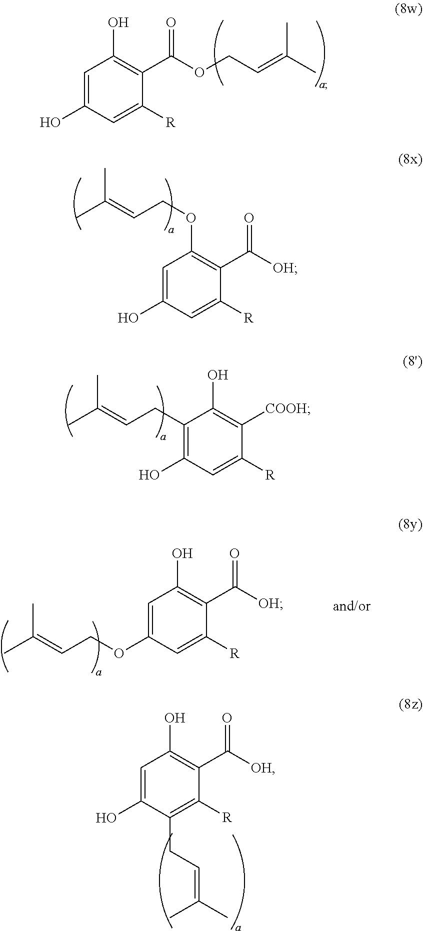

or a pharmaceutically acceptable salt, solvate, hydrate, polymorph, co-crystal, tautomer, stereoisomer, isotopically labeled derivative, or prodrug thereof, wherein a is 1, 2, 3, 4, 5, 6, 7, 8, 9, or 10.

In certain embodiments, a compound of Formula (8′) is a compound of Formula (8):

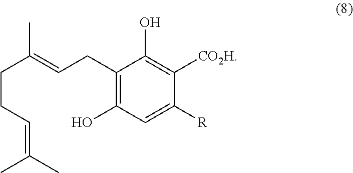

In some embodiments, R is hydrogen, an optionally substituted C1-C11 alkyl, an optionally substituted C1-C11 alkenyl, an optionally substituted C1-C11 alkynyl, or an optionally substituted C1-C11 aralkyl.

In some embodiments, a TS catalyzes oxidative cyclization of the prenyl moiety (e.g., terpene) of a compound of Formula (8) described in this application and shown in FIG. 2. In certain embodiments, a compound of Formula (8) is a compound of Formula (8a):

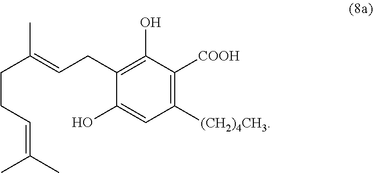

In some embodiments, the production of a compound of Formula (11) from a particular substrate may be assessed relative to the production of a compound of Formula (11) from a control substrate. In some embodiments, the production of a compound of Formula (10) from a particular substrate may be assessed relative to the production of a compound of Formula (10) from a control substrate. In some embodiments, the production of a compound of Formula (9) from a particular substrate may be assessed relative to the production of a compound of Formula (9) from a control substrate.

b. Products

In some embodiments, TS enzymes catalyze the formation of CBD-type cannabinoids, THC-type cannabinoids and/or CBC-type cannabinoids from CBG-type cannabinoids. In embodiments where CBGA is the substrate, the TS enzymes CBDAS, THCAS and CBCAS would generally catalyze the formation of cannabidiolic acid (CBDA), A9-tetrahydrocannabinolic acid (THCA) and cannabichromenic acid (CBCA), respectively. However, in some embodiments, a TS can produce more than one different product depending on reaction conditions. Product promiscuity has been noted among the Cannabis terminal synthases (e.g., Zirpel et al., J. Biotechnol. 2018 Apr. 20; 272:40-7). Without wishing to be bound by any theory, it is believed that the reaction conditions affect the protonation state and orientation of the amino acids that form the substrate binding site of the TS enzymes, which may affect the docking of the substrate and/or products of these enzymes. For example, the pH of the reaction environment may cause a THCAS or a CBDAS to produce CBCA in greater proportions than THCA or CBDAS, respectively (see, for example, U.S. Pat. No. 9,359,625 to Winnicki and Donsky, incorporated by reference in its entirety). In some embodiments, a TS has a predetermined product specificity in intracellular conditions, such as cytosolic conditions or organelle conditions. By expressing a TS with a predetermined product specificity based on intracellular conditions, in vivo products produced by a cell expressing the TS may be more predictably produced. In some embodiments, a TS produces a desired product at a pH of 5.5. In some embodiments, a TS produces a desired product at a pH of 1, 2, 3, 4, 5, 6, 7, 8, 9, 10, 11, 12, 13 or 14. In some embodiments, a TS produces a desired product at a pH that is between 4.5 and 8.0. In some embodiments, a TS produces a desired product at a pH that is between 5 and 6. In some embodiments, a TS produces a desired product at a pH that is around 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0, including all values in between. In some embodiments, the product profile of a TS is dependent on the TS's signal peptide because the signal peptide targets the TS to a particular intracellular location having particular intracellular conditions (e.g. a particular organelle) that regulate the type of product produced by the TS. Exemplary signal peptides are discussed in further detail below. Differences in the intracellular conditions can affect the activity of the TS enzymes, for example, due to variations in pH and/or differences in the folding of TS enzymes due to the presence of chaperone proteins.

A TS may be capable of using one or more substrates described in this application to produce one or more products. Non-limiting example of TS products are shown in Table 1. In some instances, a TS is capable of using one substrate to produce 1, 2, 3, 4, 5, 6, 7, 8, 9, or 10 different products. In some embodiments, a TS is capable of using more than one substrate to produce 1, 2, 3, 4, 5, 6, 7, 8, 9, or 10 different products.

In some embodiments, a TS is capable of producing a compound of Formula (X-A) and/or a compound of Formula (X-B):

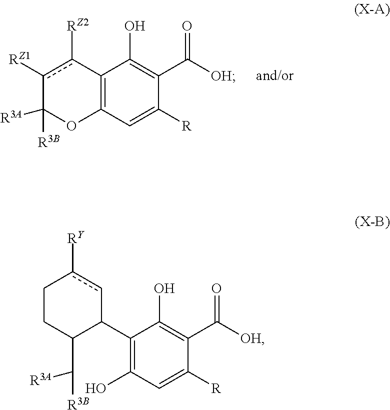

or a pharmaceutically acceptable salt, solvate, hydrate, polymorph, co-crystal, tautomer, stereoisomer, isotopically labeled derivative, or prodrug thereof;
wherein [symbol] is a double bond or a single bond, as valency permits;

R is hydrogen, optionally substituted acyl, optionally substituted alkyl, optionally substituted alkenyl, optionally substituted alkynyl, optionally substituted carbocyclyl, or optionally substituted aryl;

R^{Z1} is hydrogen, optionally substituted acyl, optionally substituted alkyl, optionally substituted alkenyl, optionally substituted alkynyl, optionally substituted carbocyclyl, or optionally substituted aryl;

R^{Z2} is hydrogen, optionally substituted acyl, optionally substituted alkyl, optionally substituted alkenyl, optionally substituted alkynyl, optionally substituted carbocyclyl, or optionally substituted aryl; or optionally, R^{Z1} and R^{Z2} are taken together with their intervening atoms to form an optionally substituted carbocyclic ring;

R^{3A} is hydrogen, optionally substituted acyl, optionally substituted alkyl, optionally substituted alkenyl, or optionally substituted alkynyl;

R^{3B} is hydrogen, optionally substituted acyl, optionally substituted alkyl, optionally substituted alkenyl, or optionally substituted alkynyl; and/or

R^{Y} is hydrogen, optionally substituted acyl, optionally substituted alkyl, optionally substituted alkenyl, or optionally substituted alkynyl.

In some embodiments, a compound of Formula (X-A) is:

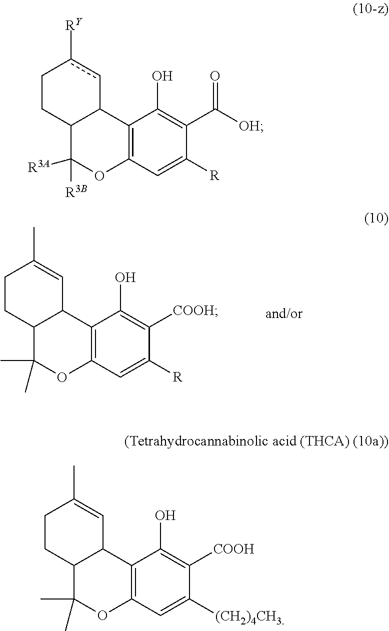

In certain embodiments, a compound of Formula (10)

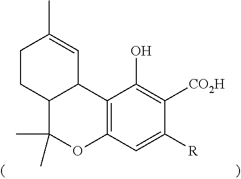

has a chiral atom labeled with * at carbon 10 and a chiral atom labeled with ** at carbon 6. In certain embodiments, in a compound of Formula (10)

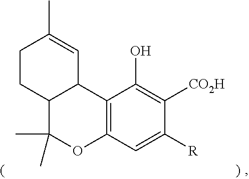

the chiral atom labeled with * at carbon 10 is of the R-configuration or S-configuration; and a chiral atom labeled with ** at carbon 6 is of the R-configuration. In certain embodiments, in a compound of Formula (10)

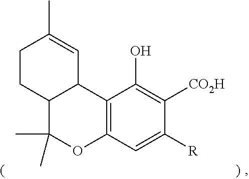

the chiral atom labeled with * at carbon 10 is of the S-configuration; and a chiral atom labeled with ** at carbon 6 is of the R-configuration or S-configuration. In certain embodiments, in a compound of Formula (10)

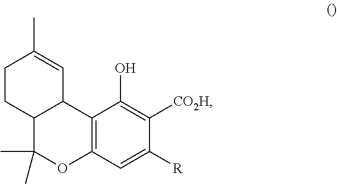

the chiral atom labeled with * at carbon 10 is of the R-configuration and a chiral atom labeled with ** at carbon 6 is of the R-configuration. In certain embodiments, a compound of Formula (10)

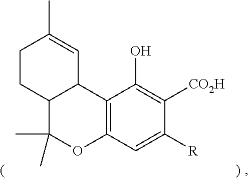

is of the formula:

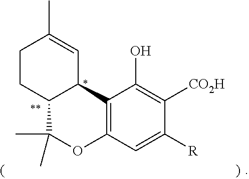

In certain embodiments, in a compound of Formula (10)

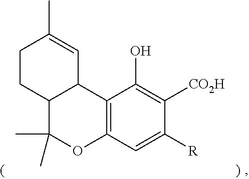

the chiral atom labeled with * at carbon 10 is of the S-configuration and a chiral atom labeled with ** at carbon 6 is of the S-configuration. In certain embodiments, a compound of Formula (10)

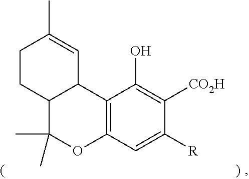

is of the formula:

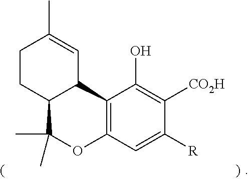

In certain embodiments, a compound of Formula (10a)

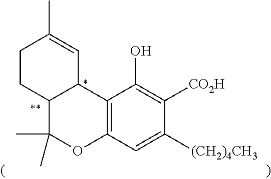

has a chiral atom labeled with * at carbon 10 and a chiral atom labeled with ** at carbon 6. In certain embodiments, in a compound of Formula (10a)

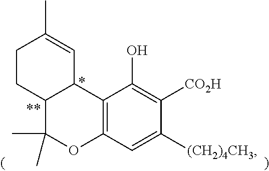

the chiral atom labeled with * at carbon 10 is of the R-configuration or S-configuration; and a chiral atom labeled with ** at carbon 6 is of the R-configuration. In certain embodiments, in a compound of Formula (10a)

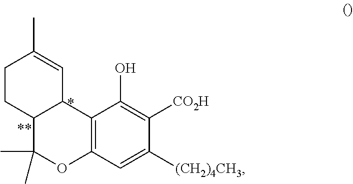

the chiral atom labeled with * at carbon 10 is of the S-configuration; and a chiral atom labeled with ** at carbon 6 is of the R-configuration or S-configuration. In certain embodiments, in a compound of Formula (10a)

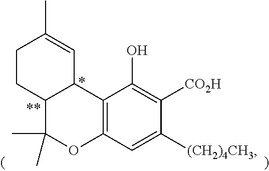

the chiral atom labeled with * at carbon 10 is of the R-configuration and a chiral atom labeled with ** at carbon 6 is of the R-configuration. In certain embodiments, a compound of Formula (10a)

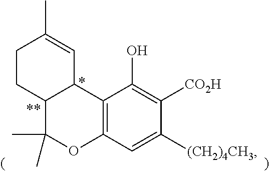

is of the formula:

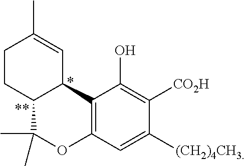

In certain embodiments, in a compound of Formula (10a)

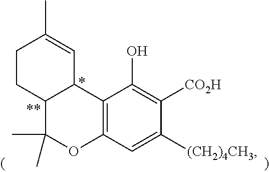

the chiral atom labeled with * at carbon 10 is of the S-configuration and a chiral atom labeled with ** at carbon 6 is of the S-configuration. In certain embodiments, a compound of Formula (10a)

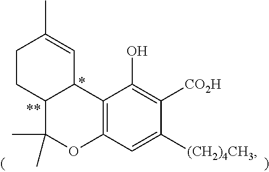

is of the formula:

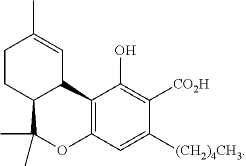

In some embodiments, a compound of Formula (X-A) is:

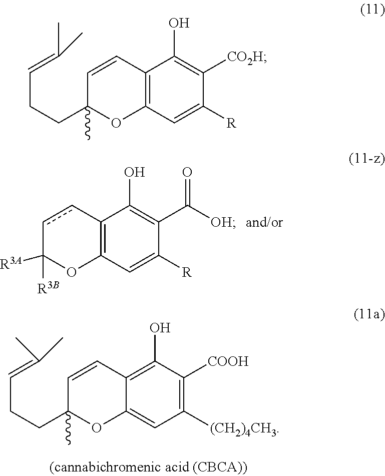

In some embodiments, a compound of Formula (X-A) is:

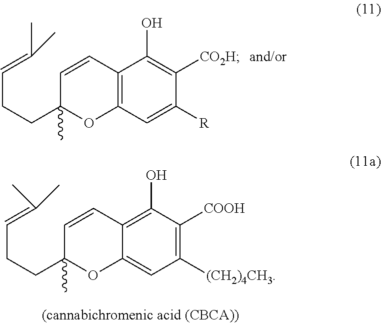

In some embodiments, a compound of Formula (X-B) is:

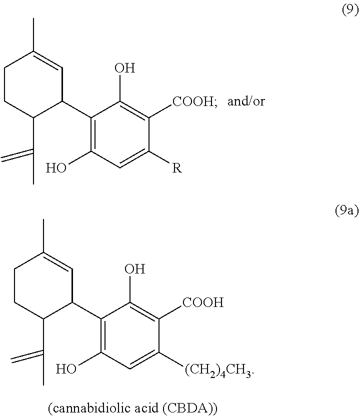

In certain embodiments, a compound of Formula (9)

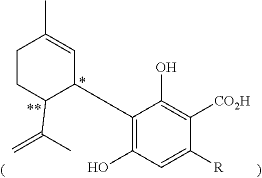

has a chiral atom labeled with * at carbon 3 and a chiral atom labeled with ** at carbon 4. In certain embodiments, in a compound of Formula (9)

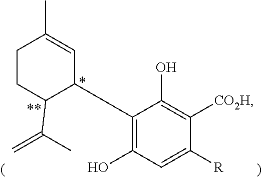

the chiral atom labeled with * at carbon 3 is of the R-configuration or S-configuration; and a chiral atom labeled with ** at carbon 4 is of the R-configuration. In certain embodiments, in a compound of Formula (9)

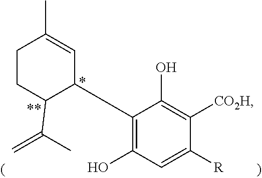

the chiral atom labeled with * at carbon 3 is of the S-configuration; and a chiral atom labeled with ** at carbon 4 is of the R-configuration or S-configuration. In certain embodiments, in a compound of Formula (9)

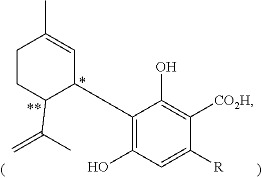

the chiral atom labeled with * at carbon 3 is of the R-configuration and a chiral atom labeled with ** at carbon 4 is of the R-configuration. In certain embodiments, a compound of Formula (9)

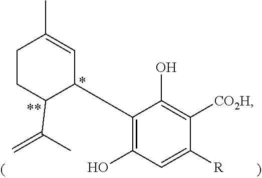

is of the formula:

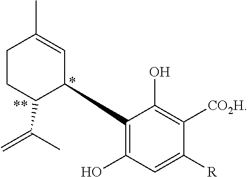

In certain embodiments, in a compound of Formula (9)

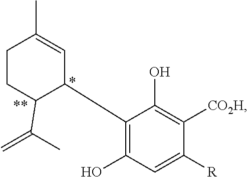

the chiral atom labeled with * at carbon 3 is of the S-configuration and a chiral atom labeled with ** at carbon 4 is of the S-configuration. In certain embodiments, a compound of Formula (9)

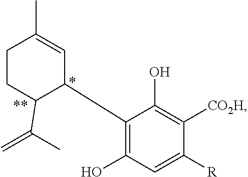

is of the formula:

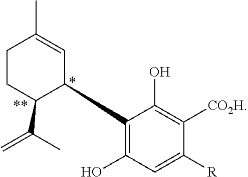

In certain embodiments, a compound of Formula (9a) (CBDA)

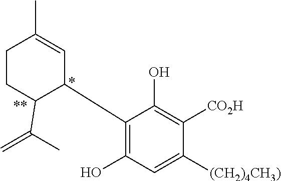

has a chiral atom labeled with * at carbon 3 and a chiral atom labeled with ** at carbon 4. In certain embodiments, in a compound of Formula (9a)

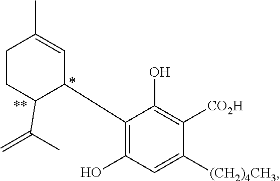

the chiral atom labeled with * at carbon 3 is of the R-configuration or S-configuration; and a chiral atom labeled with ** at carbon 4 is of the R-configuration. In certain embodiments, in a compound of Formula (9a)

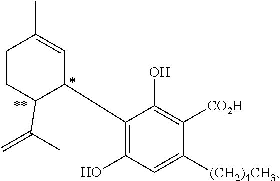

the chiral atom labeled with * at carbon 3 is of the S-configuration; and a chiral atom labeled with ** at carbon 4 is of the R-configuration or S-configuration. In certain embodiments, in a compound of Formula (9a)

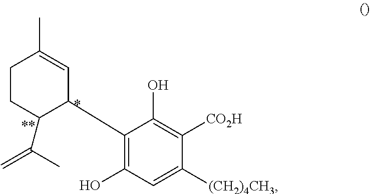

the chiral atom labeled with * at carbon 3 is of the R-configuration and a chiral atom labeled with ** at carbon 4 is of the R-configuration. In certain embodiments, a compound of Formula (9a)

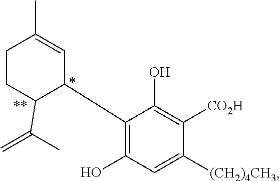

is of the formula:

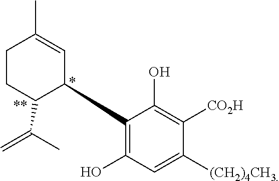

In certain embodiments, in a compound of Formula (9a)

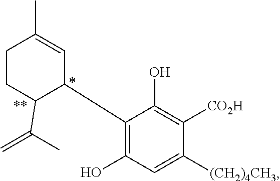

the chiral atom labeled with * at carbon 3 is of the S-configuration and a chiral atom labeled with ** at carbon 4 is of the S-configuration. In certain embodiments, a compound of Formula (9a)

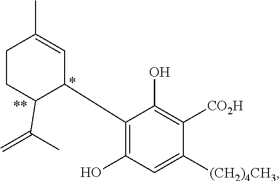

is of the formula:

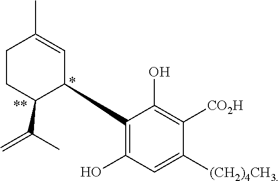

In some embodiments, as shown in FIG. 2, a TS is capable of producing a cannabinoid from the product of a PT, including, without limitation, an enzyme capable of producing a compound of Formula (9), (10), or (11):

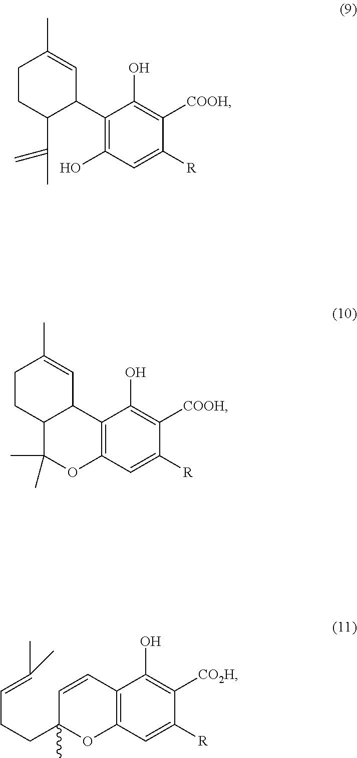

or a pharmaceutically acceptable salt, solvate, hydrate, polymorph, co-crystal, tautomer, stereoisomer, isotopically labeled derivative, or prodrug thereof, wherein R is hydrogen, optionally substituted acyl, optionally substituted alkyl, optionally substituted alkenyl, optionally substituted alkynyl, optionally substituted carbocyclyl, or optionally substituted aryl; produced from a compound of Formula (8′):

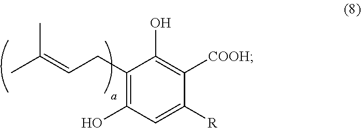

wherein a is 1, 2, 3, 4, 5, 6, 7, 8, 9, or 10; and R is hydrogen, optionally substituted acyl, optionally substituted alkyl, optionally substituted alkenyl, optionally substituted alkynyl, optionally substituted carbocyclyl, or optionally substituted aryl; or using any other substrate. In certain embodiments, a compound of Formula (8′) is a compound of Formula (8):

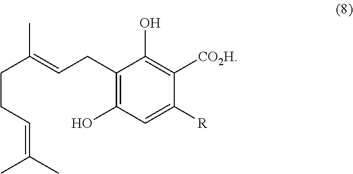

In certain embodiments, a compound of Formula (9), (10), or (11) is produced using a TS from a substrate compound of Formula (8′) (e.g., compound of Formula (8)), for example. Non-limiting examples of substrate compounds of Formula (8′) include but are not limited to cannabigerolic acid (CBGA), cannabigerovarinic acid (CBGVA), or cannabinerolic acid. In certain embodiments, at least one of the hydroxyl groups of the product compounds of Formula (9), (10), or (11) is further methylated. In certain embodiments, a compound of Formula (9) is methylated to form a compound of Formula (12):

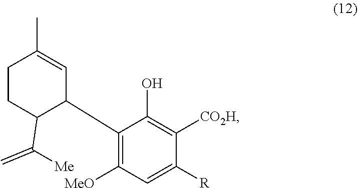

or a pharmaceutically acceptable salt, solvate, hydrate, polymorph, co-crystal, tautomer, stereoisomer, isotopically labeled derivative, or prodrug thereof.

Any of the enzymes, host cells, and methods described in this application may be used for the production of cannabinoids and cannabinoid precursors, such as those provided in Table 1. In general, the term “production” is used to refer to the generation of one or more products (e.g., products of interest and/or by-products/off-products), for example, from a particular substrate or reactant. The amount of production may be evaluated at any one or more steps of a pathway, such as a final product or an intermediate product, using metrics familiar to one of ordinary skill in the art. For example, the amount of production may be assessed for a single enzymatic reaction (e.g., conversion of a compound of Formula (8) to a compound of Formula (11) by a TS). Alternatively or in addition, the amount of production may be assessed for a series of enzymatic reactions (e.g., the biosynthetic pathway shown in FIG. 1 and/or FIG. 2). Production may be assessed by any metrics known in the art, for example, by assessing volumetric productivity, enzyme kinetics/reaction rate, specific productivity biomass-specific productivity, titer, yield, and total titer of one or more products (e.g., products of interest and/or by-products/off-products).

In some embodiments, the metric used to measure production may depend on whether a continuous process is being monitored (e.g., several cannabinoid biosynthesis steps are used in combination) or whether a particular end product is being measured. For example, in some embodiments, metrics used to monitor production by a continuous process may include volumetric productivity, enzyme kinetics and reaction rate. In some embodiments, metrics used to monitor production of a particular product may include specific productivity, biomass-specific productivity, titer, yield, and/or total titer of one or more products (e.g., products of interest and/or by-products/off-products).

Production of one or more products (e.g., products of interest and/or by-products/off-products) may be assessed indirectly, for example by determining the amount of a substrate remaining following termination of the reaction/fermentation. For example, for a TS that catalyzes the formation of products (e.g., a compound of Formula (11), including cannabichromenic acid (CBCA) (Formula (11a)) from a compound of Formula (8), including CBGA (Formula 8(a))), production of the products may be assessed by quantifying the compound of Formula (11) directly or by quantifying the amount of substrate remaining following the reaction (e.g., amount of the compound of Formula (8)). For a TS that catalyzes the formation of products (e.g., a compound of Formula (10), including tetrahydrocannabinolic acid (THCA) (Formula (10a)) from a compound of Formula (8), including CBGA (Formula 8(a))), production of the products may be assessed by quantifying the compound of Formula (10) directly or by quantifying the amount of substrate remaining following the reaction (e.g., amount of the compound of Formula (8)). For a TS that catalyzes the formation of products (e.g., a compound of Formula (9), including cannabidiolic acid (CBDA) (Formula (9a)) from a compound of Formula (8), including CBGA (Formula 8(a))), production of the products may be assessed by quantifying the compound of Formula (9) directly or by quantifying the amount of substrate remaining following the reaction (e.g., amount of the compound of Formula (8)).

In some embodiments, a TS that exhibits high production of by-products but low production of a desired product may still be used, for example if one or more amino acid substitutions, insertions, and/or deletions are introduced into the TS to shift production to the desired product, or if the TS can be expressed at locations where reaction conditions favor the production of the desired product. In some embodiments, the TS is a THCAS or has THCAS activity. Non-limiting by-products of a THCAS include compounds of Formulae (9) and (11) and a product resulting from the terpene of a compound of Formula (8) cyclizing with the other open —OH group (at carbon 1). In some embodiments, the TS is a CBDAS or has CBDAS activity. Non-limiting by-products of a CBDAS include compounds of Formulae (10) and (11) and a product resulting from the terpene of a compound of Formula (8) cyclizing with the other open —OH group (at carbon 1). In some embodiments, the TS is a CBCAS or has CBCAS activity. Non-limiting by-products of a CBCAS include compounds of Formula (9) or (10) and a product resulting from the terpene of a compound of Formula (8) cyclizing with the other open —OH group (at carbon 1). The carbons in a compound of Formula (8) may be numbered as follows:

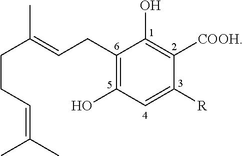

### See, e.g., Hanuš et al., Nat Prod Rep. (2016) November 23; 33(12):1357-1392.

In some embodiments, the production of a product (e.g., product of interest and/or by-product/off-product) by a particular TS may be assessed as relative production, for example relative to a control TS. In some embodiments, the production of a product by a particular host cell may be assessed relative to a control host cell.

In some embodiments, a TS or a host cell associated with the disclosure may be capable of producing a product at a higher titer or yield relative to a control. In some embodiments, a TS may be capable of producing a product at a faster rate (e.g., higher productivity) relative to a control. In some embodiments, a TS may have preferential binding and/or activity towards one substrate relative to another substrate. In some embodiments, a TS may preferentially produce one product relative to another product.

In some embodiments, a TS may produce at least 0.0001 μg/L, at least 0.001 μg/L, at least 0.01 μg/L, at least 0.02 μg/L, at least 0.03 μg/L, at least 0.04 μg/L, at least 0.05 μg/L, at least 0.06 μg/L, at least 0.07 μg/L, at least 0.08 μg/L, at least 0.09 μg/L, at least 0.1 μg/L, at least 0.11 μg/L, at least 0.12 μg/L, at least 0.13 μg/L, at least 0.14 μg/L, at least 0.15 μg/L, at least 0.16 μg/L, at least 0.17 μg/L, at least 0.18 μg/L, at least 0.19 μg/L, at least 0.2 μg/L, at least 0.21 μg/L, at least 0.22 μg/L, at least 0.23 μg/L, at least 0.24 μg/L, at least 0.25 μg/L, at least 0.26 μg/L, at least 0.27 μg/L, at least 0.28 μg/L, at least 0.29 μg/L, at least 0.3 μg/L, at least 0.31 μg/L, at least 0.32 μg/L, at least 0.33 μg/L, at least 0.34 μg/L, at least 0.35 μg/L, at least 0.36 μg/L, at least 0.37 μg/L, at least 0.38 μg/L, at least 0.39 μg/L, at least 0.4 μg/L, at least 0.41 μg/L, at least 0.42 μg/L, at least 0.43 μg/L, at least 0.44 μg/L, at least 0.45 μg/L, at least 0.46 μg/L, at least 0.47 μg/L, at least 0.48 μg/L, at least 0.49 μg/L, at least 0.5 μg/L, at least 0.51 μg/L, at least 0.52 μg/L, at least 0.53 μg/L, at least 0.54 μg/L, at least 0.55 μg/L, at least 0.56 μg/L, at least 0.57 μg/L, at least 0.58 μg/L, at least 0.59 μg/L, at least 0.6 μg/L, at least 0.61 μg/L, at least 0.62 μg/L, at least 0.63 μg/L, at least 0.64 μg/L, at least 0.65 μg/L, at least 0.66 μg/L, at least 0.67 μg/L, at least 0.68 μg/L, at least 0.69 μg/L, at least 0.7 μg/L, at least 0.71 μg/L, at least 0.72 μg/L, at least 0.73 μg/L, at least 0.74 μg/L, at least 0.75 μg/L, at least 0.76 μg/L, at least 0.77 μg/L, at least 0.78 μg/L, at least 0.79 μg/L, at least 0.8 μg/L, at least 0.81 μg/L, at least 0.82 μg/L, at least 0.83 μg/L, at least 0.84 μg/L, at least 0.85 μg/L, at least 0.86 μg/L, at least 0.87 μg/L, at least 0.88 μg/L, at least 0.89 μg/L, at least 0.9 μg/L, at least 0.91 μg/L, at least 0.92 μg/L, at least 0.93 μg/L, at least 0.94 μg/L, at least 0.95 μg/L, at least 0.96 μg/L, at least 0.97 μg/L, at least 0.98 μg/L, at least 0.99 μg/L, at least 1 μg/L, at least 1.1 μg/L, at least 1.2 μg/L, at least 1.3 μg/L, at least 1.4 μg/L, at least 1.5 μg/L, at least 1.6 μg/L, at least 1.7 μg/L, at least 1.8 μg/L, at least 1.9 μg/L, at least 2 μg/L, at least 2.1 μg/L, at least 2.2 μg/L, at least 2.3 μg/L, at least 2.4 μg/L, at least 2.5 μg/L, at least 2.6 μg/L, at least 2.7 μg/L, at least 2.8 μg/L, at least 2.9 μg/L, at least 3 μg/L, at least 3.1 μg/L, at least 3.2 μg/L, at least 3.3 μg/L, at least 3.4 μg/L, at least 3.5 μg/L, at least 3.6 μg/L, at least 3.7 μg/L, at least 3.8 μg/L, at least 3.9 μg/L, at least 4 μg/L, at least 4.1 μg/L, at least 4.2 μg/L, at least 4.3 μg/L, at least 4.4 μg/L, at least 4.5 μg/L, at least 4.6 μg/L, at least 4.7 μg/L, at least 4.8 μg/L, at least 4.9 μg/L, at least 5 μg/L, at least 5.1 μg/L, at least 5.2 μg/L, at least 5.3 μg/L, at least 5.4 μg/L, at least 5.5 μg/L, at least 5.6 μg/L, at least 5.7 μg/L, at least 5.8 μg/L, at least 5.9 μg/L, at least 6 μg/L, at least 6.1 μg/L, at least 6.2 μg/L, at least 6.3 μg/L, at least 6.4 μg/L, at least 6.5 μg/L, at least 6.6 μg/L, at least 6.7 μg/L, at least 6.8 μg/L, at least 6.9 μg/L, at least 7 μg/L, at least 7.1 μg/L, at least 7.2 μg/L, at least 7.3 μg/L, at least 7.4 μg/L, at least 7.5 μg/L, at least 7.6 μg/L, at least 7.7 μg/L, at least 7.8 μg/L, at least 7.9 μg/L, at least 8 μg/L, at least 8.1 μg/L, at least 8.2 μg/L, at least 8.3 μg/L, at least 8.4 μg/L, at least 8.5 μg/L, at least 8.6 μg/L, at least 8.7 μg/L, at least 8.8 μg/L, at least 8.9 μg/L, at least 9 μg/L, at least 9.1 μg/L, at least 9.2 μg/L, at least 9.3 μg/L, at least 9.4 μg/L, at least 9.5 μg/L, at least 9.6 μg/L, at least 9.7 μg/L, at least 9.8 μg/L, at least 9.9 μg/L, at least 10 μg/L, at least 10.1 μg/L, at least 10.2 μg/L, at least 10.3 μg/L, at least 10.4 μg/L, at least 10.5 μg/L, at least 10.6 μg/L, at least 10.7 μg/L, at least 10.8 μg/L, at least 10.9 μg/L, at least 11 μg/L, at least 11.1 μg/L, at least 11.2 μg/L, at least 11.3 μg/L, at least 11.4 μg/L, at least 11.5 μg/L, at least 11.6 μg/L, at least 11.7 μg/L, at least 11.8 μg/L, at least 11.9 μg/L, at least 12 μg/L, at least 12.1 μg/L, at least 12.2 μg/L, at least 12.3 μg/L, at least 12.4 μg/L, at least 12.5 μg/L, at least 12.6 μg/L, at least 12.7 μg/L, at least 12.8 μg/L, at least 12.9 μg/L, at least 13 μg/L, at least 13.1 μg/L, at least 13.2 μg/L, at least 13.3 μg/L, at least 13.4 μg/L, at least 13.5 μg/L, at least 13.6 μg/L, at least 13.7 μg/L, at least 13.8 μg/L, at least 13.9 μg/L, at least 14 μg/L, at least 14.1 μg/L, at least 14.2 μg/L, at least 14.3 μg/L, at least 14.4 μg/L, at least 14.5 μg/L, at least 14.6 μg/L, at least 14.7 μg/L, at least 14.8 μg/L, at least 14.9 μg/L, at least 15 μg/L, at least 15.1 μg/L, at least 15.2 μg/L, at least 15.3 μg/L, at least 15.4 μg/L, at least 15.5 μg/L, at least 15.6 μg/L, at least 15.7 μg/L, at least 15.8 μg/L, at least 15.9 μg/L, at least 16 μg/L, at least 16.1 μg/L, at least 16.2 μg/L, at least 16.3 μg/L, at least 16.4 μg/L, at least 16.5 μg/L, at least 16.6 μg/L, at least 16.7 μg/L, at least 16.8 μg/L, at least 16.9 μg/L, at least 17 μg/L, at least 17.1 μg/L, at least 17.2 μg/L, at least 17.3 μg/L, at least 17.4 μg/L, at least 17.5 μg/L, at least 17.6 μg/L, at least 17.7 μg/L, at least 17.8 μg/L, at least 17.9 μg/L, at least 18 μg/L, at least 18.1 μg/L, at least 18.2 μg/L, at least 18.3 μg/L, at least 18.4 μg/L, at least 18.5 μg/L, at least 18.6 μg/L, at least 18.7 μg/L, at least 18.8 μg/L, at least 18.9 μg/L, at least 19 μg/L, at least 19.1 μg/L, at least 19.2 μg/L, at least 19.3 μg/L, at least 19.4 μg/L, at least 19.5 μg/L, at least 19.6 μg/L, at least 19.7 μg/L, at least 19.8 μg/L, at least 19.9 μg/L, at least 20 μg/L, at least 25 μg/L, at least 30 μg/L, at least 35 μg/L, at least 40 μg/L, at least 45 μg/L, at least 50 μg/L, at least 55 μg/L, at least 60 μg/L, at least 65 μg/L, at least 70 μg/L, at least 75 μg/L, at least 80 μg/L, at least 85 μg/L, at least 90 μg/L, at least 95 μg/L, at least 100 μg/L, at least 105 μg/L, at least 110 μg/L, at least 115 μg/L, at least 120 μg/L, at least 125 μg/L, at least 130 μg/L, at least 135 μg/L, at least 140 μg/L, at least 145 μg/L, at least 150 μg/L, at least 155 μg/L, at least 160 μg/L, at least 165 μg/L, at least 170 μg/L, at least 175 μg/L, at least 180 μg/L, at least 185 μg/L, at least 190 μg/L, at least 195 μg/L, at least 200 μg/L, at least 205 μg/L, at least 210 μg/L, at least 215 μg/L, at least 220 μg/L, at least 225 μg/L, at least 230 μg/L, at least 235 μg/L, at least 240 μg/L, at least 245 μg/L, at least 250 μg/L, at least 255 μg/L, at least 260 μg/L, at least 265 μg/L, at least 270 μg/L, at least 275 μg/L, at least 280 μg/L, at least 285 μg/L, at least 290 μg/L, at least 295 μg/L, at least 300 μg/L, at least 305 μg/L, at least 310 μg/L, at least 315 μg/L, at least 320 μg/L, at least 325 μg/L, at least 330 μg/L, at least 335 μg/L, at least 340 μg/L, at least 345 μg/L, at least 350 μg/L, at least 355 μg/L, at least 360 μg/L, at least 365 μg/L, at least 370 μg/L, at least 375 μg/L, at least 380 μg/L, at least 385 μg/L, at least 390 μg/L, at least 395 μg/L, at least 400 μg/L, at least 405 μg/L, at least 410 μg/L, at least 415 μg/L, at least 420 μg/L, at least 425 μg/L, at least 430 μg/L, at least 435 μg/L, at least 440 μg/L, at least 445 μg/L, at least 450 μg/L, at least 455 μg/L, at least 460 μg/L, at least 465 μg/L, at least 470 μg/L, at least 475 μg/L, at least 480 μg/L, at least 485 μg/L, at least 490 μg/L, at least 495 μg/L, at least 500 μg/L, at least 600 μg/L, at least 700 μg/L, at least 800 μg/L, at least 900 μg/L, at least 1,000 μg/L, at least 2,000 μg/L, at least 3,000 μg/L, at least 4,000 μg/L, at least 5,000 μg/L, at least 6,000 μg/L, at least 7,000 μg/L, at least 8,000 μg/L, at least 9,000 μg/L, at least 10,000 μg/L, at least 11,000 μg/L, at least 12,000 μg/L, at least 13,000 μg/L, at least 14,000 μg/L, at least 15,000 μg/L, at least 16,000 μg/L, at least 17,000 μg/L, at least 18,000 μg/L, at least 19,000 μg/L, at least 20,000 μg/L, at least 21,000 μg/L, at least 22,000 μg/L, at least 23,000 μg/L, at least 24,000 μg/L, at least 25,000 μg/L, at least 26,000 μg/L, at least 27,000 μg/L, at least 28,000 μg/L, at least 29,000 μg/L, at least 30,000 μg/L, at least 31,000 μg/L, at least 32,000 μg/L, at least 33,000 μg/L, at least 34,000 μg/L, at least 35,000 μg/L, at least 36,000 μg/L, at least 37,000 μg/L, at least 38,000 μg/L, at least 39,000 μg/L, at least 40,000 μg/L, at least 41,000 μg/L, at least 42,000 μg/L, at least 43,000 μg/L, at least 44,000 μg/L, at least 45,000 μg/L, at least 46,000 μg/L, at least 47,000 μg/L, at least 48,000 μg/L, at least 49,000 μg/L, at least 50,000 μg/L, at least 51,000 μg/L, at least 52,000 μg/L, at least 53,000 μg/L, at least 54,000 μg/L, at least 55,000 μg/L, at least 56,000 μg/L, at least 57,000 μg/L, at least 58,000 μg/L, at least 59,000 μg/L, at least 60,000 μg/L, at least 61,000 μg/L, at least 62,000 μg/L, at least 63,000 μg/L, at least 64,000 μg/L, at least 65,000 μg/L, at least 66,000 μg/L, at least 67,000 μg/L, at least 68,000 μg/L, at least 69,000 μg/L, at least 70,000 μg/L, at least 71,000 μg/L, at least 72,000 μg/L, at least 73,000 μg/L, at least 74,000 μg/L, at least 75,000 μg/L, at least 76,000 μg/L, at least 77,000 μg/L, at least 78,000 μg/L, at least 79,000 μg/L, at least 80,000 μg/L, at least 81,000 μg/L, at least 82,000 μg/L, at least 83,000 μg/L, at least 84,000 μg/L, at least 85,000 μg/L, at least 86,000 μg/L, at least 87,000 μg/L, at least 88,000 μg/L, at least 89,000 μg/L, at least 90,000 μg/L, at least 91,000 μg/L, at least 92,000 μg/L, at least 93,000 μg/L, at least 94,000 μg/L, at least 95,000 μg/L, at least 96,000 μg/L, at least 97,000 μg/L, at least 98,000 μg/L, at least 99,000 μg/L, at least 100,000 μg/L, at least 105,000 μg/L, at least 110,000 μg/L, at least 115,000 μg/L, at least 120,000 μg/L, at least 125,000 μg/L, at least 130,000 μg/L, at least 135,000 μg/L, at least 140,000 μg/L, at least 145,000 μg/L, at least 150,000 μg/L, at least 155,000 μg/L, at least 160,000 μg/L, at least 165,000 μg/L, at least 170,000 μg/L, at least 175,000 μg/L, at least 180,000 μg/L, at least 185,000 μg/L, at least 190,000 μg/L, at least 195,000 μg/L, at least 200,000 μg/L, at least 205,000 μg/L, at least 210,000 μg/L, at least 215,000 μg/L, at least 220,000 μg/L, at least 225,000 μg/L, at least 230,000 μg/L, at least 235,000 μg/L, at least 240,000 μg/L, at least 245,000 μg/L, at least 250,000 μg/L, at least 255,000 μg/L, at least 260,000 μg/L, at least 265,000 μg/L, at least 270,000 μg/L, at least 275,000 μg/L, at least 280,000 μg/L, at least 285,000 μg/L, at least 290,000 μg/L, at least 295,000 μg/L, at least 300,000 μg/L, at least 305,000 μg/L, at least 310,000 μg/L, at least 315,000 μg/L, at least 320,000 μg/L, at least 325,000 μg/L, at least 330,000 μg/L, at least 335,000 μg/L, at least 340,000 μg/L, at least 345,000 μg/L, at least 350,000 μg/L, at least 355,000 μg/L, at least 360,000 μg/L, at least 365,000 μg/L, at least 370,000 μg/L, at least 375,000 μg/L, at least 380,000 μg/L, at least 385,000 μg/L, at least 390,000 μg/L, at least 395,000 μg/L, at least 400,000 μg/L, at least 405,000 μg/L, at least 410,000 μg/L, at least 415,000 μg/L, at least 420,000 μg/L, at least 425,000 μg/L, at least 430,000 μg/L, at least 435,000 μg/L, at least 440,000 μg/L, at least 445,000 μg/L, at least 450,000 μg/L, at least 455,000 μg/L, at least 460,000 μg/L, at least 465,000 μg/L, at least 470,000 μg/L, at least 475,000 μg/L, at least 480,000 μg/L, at least 485,000 μg/L, at least 490,000 μg/L, at least 495,000 μg/L, at least 500,000 μg/L, at least 600,000 μg/L, at least 700,000 μg/L, at least 800,000 μg/L, at least 900,000 μg/L, or at least 1,000,000 μg/L, including all values in between, of a product described herein. In some embodiments, a product is a compound of Formula (11) (e.g., a compound of Formula (11a)). In some embodiments, a product is CBCA and/or CBCVA. In some embodiments, a product is a compound of Formula (9) (e.g., the compound of Formula (9a)). In some embodiments, a product is a compound of Formula (10) (e.g., the compound of Formula (10a)).

In some embodiments, a TS or a host cell associated with the disclosure may be capable of producing more of an amount of one or more products than produced by a control (e.g., a positive control). In some embodiments, a TS or a host cell associated with the disclosure may be capable of producing at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) of the amount of one or more products produced by a control (e.g., such as a positive control). In some embodiments, a product is CBCA and/or CBCVA. In some embodiments, a TS or a host cell associated with the disclosure may be capable of producing at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more of one or more products produced by a control (e.g., such as a positive control). In some embodiments, a product is a compound of Formula (11) (e.g., the compound of Formula (11a)). In some embodiments, a product is CBCA and/or CBCVA. In some embodiments, a product is a compound of Formula (9) (e.g., the compound of Formula (9a)). In some embodiments, a product is a compound of Formula (10) (e.g., the compound of Formula (10a)).

In some embodiments, a TS or a host cell associated with the disclosure may be capable of producing at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) of the titer or yield of one or more products produced by a control (e.g., such as a positive control). In some embodiments, a product is CBCA and/or CBCVA. In some embodiments, a TS or a host cell associated with the disclosure may be capable of producing at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) higher titer or yield of one or more products as compared to a control. In some embodiments, a product is a compound of Formula (11) (e.g., the compound of Formula (11a)). In some embodiments, a product is CBCA and/or CBCVA. In some embodiments, a product is a compound of Formula (9) (e.g., the compound of Formula (9a)). In some embodiments, a product is a compound of Formula (10) (e.g., the compound of Formula (10a)).

In some embodiments, a TS or host cell associated with the disclosure may be capable of producing one or more products at a rate that is at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) the rate of a control (e.g., such as a positive control). In some embodiments, a product is CBCA and/or CBCVA. In some embodiments, a TS may be capable of producing one or more products at a rate that is at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) faster relative to a control (e.g., such as a positive control). In some embodiments, a product is a compound of Formula (11) (e.g., a compound of Formula (11a)). In some embodiments, a product is CBCA and/or CBCVA. In some embodiments, a product is a compound of Formula (9) (e.g., the compound of Formula (9a)). In some embodiments, a product is a compound of Formula (10) (e.g., the compound of Formula (10a)).

In some embodiments, a TS or host cell associated with the disclosure may be capable of producing less of an amount of one or more products than produced by a control (e.g., a positive control). In some embodiments, a TS or host cell associated with the disclosure may be capable of producing at least 0.05% (e.g., at least 0.075%, at least 0.1% at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) less of one or more products relative to a control (e.g., such as a positive control). In some embodiments, a product is a compound of Formula (11) (e.g., the compound of Formula (11a)). In some embodiments, a product is CBCA and/or CBCVA. In some embodiments, a product is a compound of Formula (9) (e.g., the compound of Formula (9a)). In some embodiments, a product is a compound of Formula (10) (e.g., the compound of Formula (10a)).

In some embodiments, a TS or host cell associated with the disclosure may be capable of producing at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) lower titer or yield of one or more products relative to a control (e.g., such as a positive control). In some embodiments, a product is a compound of Formula (11) (e.g., the compound of Formula (11a)). In some embodiments, a product is CBCA and/or CBCVA. In some embodiments, a product is a compound of Formula (9) (e.g., the compound of Formula (9a)). In some embodiments, a product is a compound of Formula (10) (e.g., the compound of Formula (10a)).

In some embodiments, a TS or host cell associated with the disclosure may be capable of producing one or more products at a rate that is at least 0.5% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) slower relative to a control (e.g., such as a positive control). In some embodiments, a product is a compound of Formula (11) (e.g., the compound of Formula (11a)). In some embodiments, a product is CBCA and/or CBCVA. In some embodiments, a product is a compound of Formula (9) (e.g., the compound of Formula (9a)). In some embodiments, a product is a compound of Formula (10) (e.g., the compound of Formula (10a)).

In some embodiments of methods described herein involving comparison of an experimental TS to a control, the control is a wild-type reference TS. In some embodiments, the control is a wild-type C. sativa THCAS (e.g., comprising SEQ ID NO: 21). In some embodiments, the control is a wild-type C. sativa THCAS (e.g., comprising SEQ ID NO: 21) that also exhibits CBCAS activity in addition to THCAS activity. In some embodiments, the control TS is identical to an experimental TS except for the presence of one or more amino acid substitutions, insertions, or deletions within the experimental TS.

In some embodiments of methods described herein involving comparison of an experimental host cell to a control host cell, the control host cell is a host cell that does not comprise a heterologous polynucleotide encoding a TS. In some embodiments, a control host cell is a wild-type cell. In some embodiments, a control host cell is a host cell that comprises a heterologous polynucleotide encoding a wild-type C. sativa THCAS. In some embodiments, the control is a wild-type C. sativa THCAS that also exhibits CBCAS activity in addition to THCAS activity. In Cannabis, the wild-type CsTHCAS is secreted into glandular trichomes. However, as described in further detail below, it may be desirable to control the localization of a cannabinoid produced by the recombinant host cell, for example to a particular cellular compartment and/or the cellular secretory pathway. Accordingly, in some embodiments, the control is a wild-type C. sativa THCAS, that also exhibits CBCAS activity, in which the native signal sequence has been removed (e.g., as set forth in SEQ ID NO: 21) and, optionally, replaced with one or more heterologous signal sequences. In some embodiments, a control host cell is a host cell that comprises a heterologous polynucleotide comprising SEQ ID NO: 22. In some embodiments, a control host cell is genetically identical to an experimental host cell except for the the presence of one or more amino acid substitutions, insertions, or deletions within a TS that is heterologously expressed in the experimental host cell.

In some embodiments, a TS is capable of producing a mixture of products. For example, the mixture may comprise one or more compounds of Formula (11). In some embodiments, the mixture comprises a compound of Formula (9), Formula (10), and/or Formula (11). In some embodiments, at least approximately 50-100%, at least approximately 50-60%, at least approximately 60-70%, at least approximately 70-80%, at least approximately 80-90%, at least approximately 90-100%, of compounds within the product mixture are compounds of Formula (11a). In some embodiments, from about 50-100%, at least approximately 50%, at least approximately 60%, at least approximately 70%, at least approximately 80%, or at least approximately 90%, of compounds within the product mixture are CBCA. In some embodiments, from about 50-100%, at least approximately 50%, at least approximately 60%, at least approximately 70%, at least approximately 80%, or at least approximately 90%, of compounds within the product mixture are CBCVA.

In some embodiments, a TS is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times more of a compound of Formula (11a) than another compound of Formula (11), a compound of Formula (10a), a compound of Formula (9a), or any combination thereof. In some embodiments, a TS is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times less of a compound of Formula (11a) than another compound of Formula (11), a compound of Formula (10a), a compound of Formula (9a), or any combination thereof.

In some embodiments, at least approximately 50-100%, at least approximately 50-60%, at least approximately 60-70%, at least approximately 70-80%, at least approximately 80-90%, at least approximately 90-100%, of compounds within the product mixture are compounds of Formula (9a). In some embodiments, a TS is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times more of a compound of Formula (9a) than another compound of Formula (9), a compound of Formula (10a), a compound of Formula (11a), or any combination thereof. In some embodiments, a TS is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times less of a compound of Formula (9a) than another compound of Formula (9), a compound of Formula (10a), a compound of Formula (11a), or any combination thereof.

In some embodiments, at least approximately 50-100%, at least approximately 50-60%, at least approximately 60-70%, at least approximately 70-80%, at least approximately 80-90%, at least approximately 90-100%, of compounds within the product mixture are compounds of Formula (10a). In some embodiments, a TS is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times more of a compound of Formula (10a) than another compound of Formula (10), a compound of Formula (9a), a compound of Formula (11a), or any combination thereof. In some embodiments, a TS is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times less of a compound of Formula (10a) than another compound of Formula (10), a compound of Formula (9a), a compound of Formula (11a), or any combination thereof.

c. Signal Peptides

Any of the enzymes described in this application, including TSs, may comprise a signal peptide. Signal peptides, also referred to as “signal sequences,” generally comprise approximately 15-30 amino acids and are involved in regulating trafficking of a newly translated protein to a particular cellular compartment and/or the cellular secretory pathway.

In some instances, a signal peptide promotes localization of an enzyme of interest. A non-limiting example of a signal peptide that promotes localization of an enzyme of interest in intracellular spaces is the MFalpha2 signal peptide. See, e.g., the signal sequence from UniProtKB—U3N2MO (residues 1-19) and Singh et al., Nucleic Acids Res. (1983) June 25; 11(12): 4049-4063. In other instances, a signal peptide is capable of preventing a protein from being secreted from the endoplasmic reticulum (ER) and/or is capable of facilitating the return of such a protein if it is inadvertently exported. Such a signal peptide may be referred to as an “ER retentional signal.” A non-limiting example of a signal peptide that is capable of preventing a protein from being secreted from the ER and/or is capable of facilitating the return of such a protein if it is inadvertently exported is an HDEL signal peptide. See, e.g., Pelham et al., EMBO J (1988)7:1757-1762.

Non-limiting examples of signal peptides include those listed in Table 2 below. As one of ordinary skill in the art would appreciate, other signal peptides known in the art would also be compatible with aspects of the disclosure. A signal peptide may be located N-terminal or C-terminal relative to a sequence encoding an enzyme of interest. A sequence encoding an enzyme of interest may be linked to two or more signal peptides. In some embodiments, an enzyme of interest may be linked to one or more signal peptides at the N-terminus and one or more signal peptides at the C-terminus. For example, in some embodiments, the MFalpha2 signal peptide may be located N-terminal to a sequence encoding an enzyme of interest and/or the HDEL signal peptide may be located C-terminal to a sequence encoding an enzyme of interest. In other embodiments, the HDEL signal peptide may be located N-terminal to a sequence encoding an enzyme of interest and/or the MFalpha2 signal peptide may be located C-terminal to a sequence encoding an enzyme of interest.

Without wishing to be bound by any theory, it is believed that an enzyme, such as a TS enzyme, linked to the MFalpha2 signal peptide and/or the HDEL signal peptide will be localized to intracellular locations associated with the secretory pathway, such as the ER and/or the Golgi apparatus. One or more of the conditions of the secretory pathway are believed to contribute to improved activity of TS enzymes derived from C. sativa. For example, the ER and Golgi apparatus are oxidative environments, which may assist in the formation of disulphide bridges. Without wishing to be bound by any theory, signal peptides and the resulting intracellular localization of proteins containing the signal peptides may differentially impact the stability and/or half-life of proteins.

In some embodiments, a signal peptide comprises a nucleic acid or protein sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to one or more of SEQ ID NOs: 3, 4, 16-19, 31, or 32.

In some embodiments, a signal peptide comprises a sequence that differs by no more than 1, 2, 3, 4, 5, 6, 7, 8, 9, 10, 11, 12, 13, 14, 15, 16, 17, or 18 amino acids from any of SEQ ID NOs: 3, 4, 16, or 31. In some embodiments, a signal peptide comprises no more than 1, 2, 3, 4, 5, 6, 7, 8, 9, 10, 11, 12, 13, 14, 15, 16, 17, or 18 amino acid substitutions, insertions, additions, or deletions relative to the sequence of SEQ ID NOs: 3, 4, 16, or 31. In some embodiments, a signal peptide comprises SEQ ID NO: 16 or a sequence that has no more than 2 amino acid substitutions, insertions, additions, or deletions relative to the sequence of SEQ ID NO: 16. In some embodiments, a signal peptide comprises a protein sequence that differs by no more than 1, 2 or 3 amino acids from SEQ ID NO: 17. In some embodiments, a signal peptide comprises SEQ ID NO: 17 or a sequence that has no more than one amino acid substitution, insertion, addition, or deletion relative to the sequence of SEQ ID NO: 17.

A signal peptide that is located at the N-terminus of a sequence encoding an enzyme of interest may comprise a methionine at the N-terminus of the signal peptide. In some embodiments, a methionine is added to a signal peptide if the signal peptide will be located at the N-terminus of a sequence encoding an enzyme of interest. In some embodiments, a signal peptide that is normally associated with an enzyme of interest (e.g., a naturally occurring signal peptide that is present in a naturally occurring enzyme of interest) may be removed or replaced with one or more different signal peptides that are suitable for targeting the enzyme to a particular cellular compartment in a host cell of interest.

TABLE 2
Non-limiting examples of signal peptides

| | | Non-limiting example of corresponding nucleic acid |
|---|---|---|
| Name | Amino acid sequence | sequence |
| C. sativa | NCSAFSFWFVCKIIFFFLSFNI | aattgctcagcattttccttttggtttgtttgcaaaataatatttttctttct |
| THCAS | QISIA (SEQ ID NO: 4) | ctcattcaatatccaaatttcaata (SEQ ID NO: 3) |
| native signal | | |
| peptide | | |
| MFalpha2 | KFISTFLTFILAAVSVTA (SEQ | aagtttatcagtaccttcttgacctttatcttggccgctgtctccgtaaccgc |
| | ID NO: 16) | t (SEQ ID NO: 18) |
| HDEL | HDEL (SEQ ID NO: 17) | catgatgaatta (SEQ ID NO: 19) |
| C. sativa | NCSTFSFWFVCKIIFFFLSFNIQ | aattgctcaacattctccttttggtttgtttgcaaaataatatttttctttct |
| CBCAS | ISIA (SEQ ID NO: 31) | ctcattcaatatccaa atttcaatagct (SEQ ID NO: 32) |
| native signal | | |
| peptide | | |

In some embodiments, a TS is a tetrahydrocannabinolic acid synthase (THCAS), a cannabidiolic acid synthase (CBDAS), and/or a cannabichromenic acid synthase (CBCAS). As one of ordinary skill in the art would appreciate a TS could be obtained from any source, including naturally occurring sources and synthetic sources (e.g., a non-naturally occurring TS).

### Tetrahydrocannabinolic Acid Synthase (THCAS)

A host cell described in this application may comprise a TS that is a tetrahydrocannabinolic acid synthase (THCAS). As used in this application “tetrahydrocannabinolic acid synthase (THCAS)” or “Δ¹-tetrahydrocannabinolic acid (THCA) synthase” refers to an enzyme that is capable of catalyzing oxidative cyclization of a prenyl moiety (e.g., terpene) of a compound of Formula (8) to produce a ring-containing product (e.g., heterocyclic ring-containing product, carbocyclic-ring containing product) of Formula (10). In certain embodiments, a THCAS refers to an enzyme that is capable of producing Δ9-tetrahydrocannabinolic acid (Δ9-THCA, THCA, Δ9-Tetrahydro-cannabivarinic acid A (Δ9-THCVA-C3 A), THCVA, THCPA, or a compound of Formula 10(a), from a compound of Formula (8). In certain embodiments, a THCAS is capable of producing Δ⁹-tetrahydrocannabinolic acid (Δ⁹-THCA, THCA, or a compound of Formula 10(a)). In certain embodiments, a THCAS is capable of producing Δ9-tetrahydrocannabivarinic acid (Δ9-THCVA, THCVA, or a compound of Formula 10 where R is n-propyl).

In some embodiments, a THCAS may catalyze the oxidative cyclization of substrates, such as 3-prenyl-2,4-dihydroxy-6-alkylbenzoic acids. In some embodiments, a THCAS may use cannabigerolic acid (CBGA) as a substrate. In some embodiments, the THCAS produces Δ9-THCA from CBGA. In some embodiments, a THCAS may catalyze the oxidative cyclization of cannabigerovarinic acid (CBGVA). In some embodiments, a THCAS exhibits specificity for CBGA substrates as compared to other substrates. In some embodiments, a THCAS may use a compound of Formula (8) of FIG. 2 where R is C4 alkyl (e.g., n-butyl) or R is C7 alkyl (e.g., n-heptyl) as a substrate. In some embodiments, a THCAS may use a compound of Formula (8) where R is C4 alkyl (e.g., n-butyl) as a substrate. In some embodiments, a THCAS may use a compound of Formula (8) of FIG. 2 where R is C7 alkyl (e.g., n-heptyl) as a substrate. In some embodiments, the THCAS exhibits specificity for substrates that can result in THCP as a product.

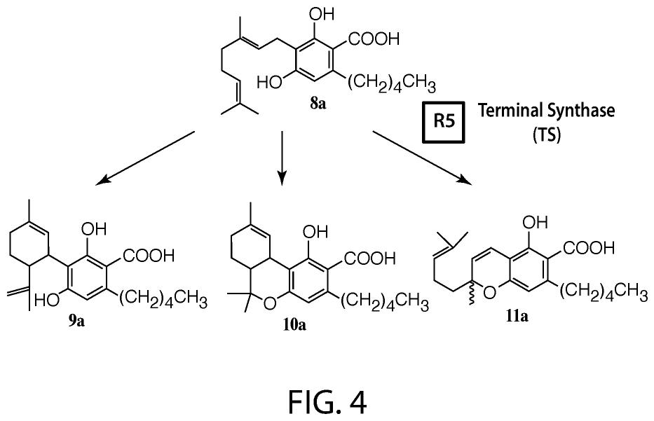

In some embodiments, a THCAS is from C. sativa. C. sativa THCAS performs the oxidative cyclization of the geranyl moiety of Cannabigerolic Acid (CBGA) (FIG. 4 Structure 8a) to form Tetrahydrocannabinolic Acid (FIG. 4 Structure 10a) using covalently bound flavin adenine dinucleotide (FAD) as a cofactor and molecular oxygen as the final electron acceptor. THCAS was first discovered and characterized by Taura et al. (JACS. 1995) following extraction of the enzyme from the leaf buds of C. sativa and confirmation of its THCA synthase activity in vitro upon the addition of CBGA as a substrate. A crystal structure of the enzyme published by Shoyama et al. (J Mol Biol. 2012 Oct. 12; 423(1):96-105) revealed that the enzyme covalently binds to a molecule of the cofactor FAD. See also, e.g., Sirikantarams et al., J. Biol. Chem. 2004 Sep. 17; 279(38):39767-39774. There are several THCAS isozymes in C. sativa.

In some embodiments, a C. sativa THCAS (Uniprot KB Accession No.: I1V0C5) comprises the amino acid sequence shown below, in which the signal peptide is underlined and bolded:

(SEQ ID NO: 20)
MNCSAFSFWFVCKIIFFFLSFNIQISIANPQENFLKCFSEYIPNNPANP
KFIYTQHDQLYMSVLNSTIQNLRFTSDTTPKPLVIVTPSNVSHIQASIL
CSKKVGLQIRTRSGGHDAEGMSYISQVPFVVVDLRNMHSIKIDVHSQTA
WVEAGATLGEVYYWINEKNENFSFPGGYCPTVGVGGHFSGGGYGALMRN
YGLAADNIIDAHLVNVDGKVLDRKSMGEDLFWAIRGGGGENFGIIAAWK
IKLVAVPSKSTIFSVKKNMEIHGLVKLFNKWQNIAYKYDKDLVLMTHFI
TKNITDNHGKNKTTVHGYFSSIFHGGVDSLVDLMNKSFPELGIKKTDCK
EFSWIDTTIFYSGVVNFNTANFKKEILLDRSAGKKTAFSIKLDYVKKPI
PETAMVKILEKLYEEDVGVGMYVLYPYGGIMEEISESAIPFPHRAGIMY
ELWYTASWEKQEDNEKHINWVRSVYNFTTPYVSQNPRLAYLNYRDLDLG
KTNPESPNNYTQARIWGEKYFGKNFNRLVKVKTKADPNNFFRNEQSIPP
LPPHHH.

In some embodiments, a THCAS comprises the sequence shown below:

(SEQ ID NO: 21)
NPQENFLKCFSEYIPNNPANPKFIYTQHDQLYMSVLNSTIQNLRFTSDT
TPKPLVIVTPSNVSHIQASILCSKKVGLQIRTRSGGHDAEGMSYISQVP
FVVVDLRNMHSIKIDVHSQTAWVEAGATLGEVYYWINEKNENFSFPGGY
CPTVGVGGHFSGGGYGALMRNYGLAADNIIDAHLVNVDGKVLDRKSMGE
DLFWAIRGGGGENFGIIAAWKIKLVAVPSKSTIFSVKKNMEIHGLVKLF
NKWQNIAYKYDKDLVLMTHFITKNITDNHGKNKTTVHGYFSSIFHGGVD
SLVDLMNKSFPELGIKKTDCKEFSWIDTTIFYSGVVNFNTANFKKEILL
DRSAGKKTAFSIKLDYVKKPIPETAMVKILEKLYEEDVGVGMYVLYPYG
GIMEEISESAIPFPHRAGIMYELWYTASWEKQEDNEKHINWVRSVYNFT
TPYVSQNPRLAYLNYRDLDLGKTNPESPNNYTQARIWGEKYFGKNFNRL
VKVKTKADPNNFFRNEQSIPPLPPHHH.

A non-limiting example of a nucleotide sequence encoding SEQ ID NO: 21 is:

(SEQ ID NO: 22)
aacccgcaagaaaactttctaaaatgcttttctgaatacattcctaacaaccctgccaacccgaagtttatctacacacaacacgatcaat
tgtatatgagcgtgttgaatagtacaatacagaacctgaggtttacatccgacacaacgccgaaaccgctagtgatcgtcacaccctcca
acgtaagccacattcaggcaagcattttatgcagcaagaaagtcggactgcagataaggacgaggtccggaggacacgacgccgaa
gggatgagctatatctcccaggtaccttttgtggtggtagacttgagaaatatgcactctatcaagatagacgttcactcccaaaccgctt
gggttgaggcgggagccacccttggtgaggtctactactggatcaacgaaaagaatgaaaattttagctttcctgggggatattgccca
actgtaggtgttggcggccacttctcaggaggcggttatggggccttgatgcgtaactacggacttgcggccgacaacattatagacg
cacatctagtgaatgtagacggcaaagttttagacaggaagagcatgggtgaggatcttttttgggcaattagaggcggagggggaga
aaattttggaattatcgctgcttggaaaattaagctagttgcggtaccgagcaaaagcactatattctctgtaaaaaagaacatggagata
catggtttggtgaagctttttaataagtggcaaaacatcgcgtacaagtacgacaaagatctggttctgatgacgcattttataacgaaaa
atatcaccgacaaccacggaaaaaacaaaaccacagtacatggctacttctctagtatatttcatgggggagtcgattctctggttgattt
aatgaacaaatcattcccagagttgggtataaagaagacagactgtaaggagttctcttggattgacacaactatattctattcaggcgta
gtcaactttaacacggcgaatttcaaaaaagagatccttctggacagatccgcaggtaagaaaactgcgttctctatcaaattggactatg
tgaagaagcctattcccgaaaccgcgatggtcaagatacttgagaaattatacgaggaagatgtgggagttggaatgtacgtactttatc
cctatggtgggataatggaagaaatcagcgagagcgccattccatttccccatcgtgccggcatcatgtacgagctgtggtatactgcg
agttgggagaagcaagaagacaacgaaaagcacattaactgggtcagatcagtttacaatttcaccaccccatacgtgtcccagaatc
cgcgtctggcttacttgaactaccgtgatcttgacctgggtaaaacgaacccggagtcacccaacaattacactcaagctagaatctgg
ggagagaaatactttgggaagaacttcaacaggttagtaaaggttaaaaccaaggcagatccaaacaacttttttagaaatgaacaatc
cattcccccgctacccccgcaccatcac.

In some embodiments, a THCAS comprises the amino acid sequence shown below, in which signal peptides are underlined and bolded:

(SEQ ID NO: 23)
MKFISTFLTFILAAVSVTANPQENFLKCFSEYIPNNPANPKFIYTQHDQ
LYMSVLNSTIQNLRFTSDTTPKPLVIVTPSNVSHIQASILCSKKVGLQI
RTRSGGHDAEGMSYISQVPFVVVDLRNMHSIKIDVHSQTAWVEAGATLG
EVYYWINEKNENFSFPGGYCPTVGVGGHFSGGGYGALMRNYGLAADNII
DAHLVNVDGKVLDRKSMGEDLFWAIRGGGGENFGIIAAWKIKLVAVPSK
STIFSVKKNMEIHGLVKLFNKWQNIAYKYDKDLVLMTHFITKNITDNHG
KNKTTVHGYFSSIFHGGVDSLVDLMNKSFPELGIKKTDCKEFSWIDTTI
FYSGVVNFNTANFKKEILLDRSAGKKTAFSIKLDYVKKPIPETAMVKIL
EKLYEEDVGVGMYVLYPYGGIMEEISESAIPFPHRAGIMYELWYTASWE
KQEDNEKHINWVRSVYNFTTPYVSQNPRLAYLNYRDLDLGKTNPESPNN
YTQARIWGEKYFGKNFNRLVKVKTKADPNNFFRNEQSIPPLPPHHHHDE
L.

A non-limiting example of a nucleotide sequence encoding SEQ ID NO: 23, in which sequences encoding signal peptides are underlined and bolded, is shown below:

(SEQ ID NO: 24)
atgaagtttatcagtaccttcttgacctttatcttggccgctgtctccgtaaccgctaacccgcaagaaaactttctaaaatgcttttct
gaatacattcctaacaaccctgccaacccgaagtttatctacacacaacacgatcaattgtatatgagcgtgttgaatagtacaatacaga
acctgaggtttacatccgacacaacgccgaaaccgctagtgatcgtcacaccctccaacgtaagccacattcaggcaagcattttatgc
agcaagaaagtcggactgcagataaggacgaggtccggaggacacgacgccgaagggatgagctatatctcccaggtaccttttgt
ggtggtagacttgagaaatatgcactctatcaagatagacgttcactcccaaaccgcttgggttgaggcgggagccacccttggtgag
gtctactactggatcaacgaaaagaatgaaaattttagctttcctgggggatattgcccaactgtaggtgttggcggccacttctcaggag
gcggttatggggccttgatgcgtaactacggacttgcggccgacaacattatagacgcacatctagtgaatgtagacggcaaagtttta
gacaggaagagcatgggtgaggatcttttttgggcaattagaggcggagggggagaaaattttggaattatcgctgcttggaaaattaa
gctagttgcggtaccgagcaaaagcactatattctctgtaaaaaagaacatggagatacatggtttggtgaagctttttaataagtggcaa
aacatcgcgtacaagtacgacaaagatctggttctgatgacgcattttataacgaaaaatatcaccgacaaccacggaaaaaacaaaa
ccacagtacatggctacttctctagtatatttcatgggggagtcgattctctggttgatttaatgaacaaatcattcccagagttgggtata
aagaagacagactgtaaggagttctcttggattgacacaactatattctattcaggcgtagtcaactttaacacggcgaatttcaaaaaaga
gatccttctggacagatccgcaggtaagaaaactgcgttctctatcaaattggactatgtgaagaagcctattcccgaaaccgcgatggt
caagatacttgagaaattatacgaggaagatgtgggagttggaatgtacgtactttatccctatggtgggataatggaagaaatcagcga
gagcgccattccatttccccatcgtgccggcatcatgtacgagctgtggtatactgcgagttgggagaagcaagaagacaacgaaaa
gcacattaactgggtcagatcagtttacaatttcaccaccccatacgtgtcccagaatccgcgtctggcttacttgaactaccgtgatcttg
acctgggtaaaacgaacccggagtcacccaacaattacactcaagctagaatctggggagagaaatactttgggaagaacttcaaca
ggttagtaaaggttaaaaccaaggcagatccaaacaacttttttagaaatgaacaatccattcccccgctacccccgcaccatcaccat
gatgaatta.

In some embodiments, a C. sativa THCAS comprises the amino acid sequence set forth in UniProtKB—Q8GTB6 (SEQ ID NO: 14) in which the signal peptide is underlined and bolded:

(SEQ ID NO: 14)
MNCSAFSFWFVCKIIFFFLSFHIQISIANPRENFLKCFSKHIPNNVANPK
LVYTQHDQLYMSILNSTIQNLRFISDTTPKPLVIVTPSNNSHIQATILCS
KKVGLQIRTRSGGHDAEGMSYISQVPFVVVDLRNMHSIKIDVHSQTAWVE
AGATLGEVYYWINEKNENLSFPGGYCPTVGVGGHFSGGGYGALMRNYGLA
ADNIIDAHLVNVDGKVLDRKSMGEDLFWAIRGGGGENFGIIAAWKIKLVA
VPSKSTIFSVKKNMEIHGLVKLFNKWQNIAYKYDKDLVLMTHFITKNITD
NHGKNKTTVHGYFSSIFHGGVDSLVDLMNKSFPELGIKKTDCKEFSWIDT
TIFYSGVVNFNTANFKKEILLDRSAGKKTAFSIKLDYVKKPIPETAMVKI
LEKLYEEDVGAGMYVLYPYGGIMEEISESAIPFPHRAGIMYELWYTASWE
KQEDNEKHINWVRSVYNFTTPYVSQNPRLAYLNYRDLDLGKTNHASPNNY
TQARIWGEKYFGKNFNRLVKVKTKVDPNNFFRNEQSIPPLPPHHH.

In some embodiments, a THCAS comprises the sequence shown below:

(SEQ ID NO: 214)
NPRENFLKCFSKHIPNNVANPKLVYTQHDQLYMSILNSTIQNLRFISDTT
PKPLVIVTPSNNSHIQATILCSKKVGLQIRTRSGGHDAEGMSYISQVPFV
VVDLRNMHSIKIDVHSQTAWVEAGATLGEVYYWINEKNENLSFPGGYCPT
VGVGGHFSGGGYGALMRNYGLAADNIIDAHLVNVDGKVLDRKSMGEDLFW
AIRGGGGENFGIIAAWKIKLVAVPSKSTIFSVKKNMEIHGLVKLFNKWQN
IAYKYDKDLVLMTHFITKNITDNHGKNKTTVHGYFSSIFHGGVDSLVDLM
NKSFPELGIKKTDCKEFSWIDTTIFYSGVVNFNTANFKKEILLDRSAGKK
TAFSIKLDYVKKPIPETAMVKILEKLYEEDVGAGMYVLYPYGGIMEEISE
SAIPFPHRAGIMYELWYTASWEKQEDNEKHINWVRSVYNFTTPYVSQNPR
LAYLNYRDLDLGKTNHASPNNYTQARIWGEKYFGKNFNRLVKVKTKVDPN
NFFRNEQSIPPLPPHHH

Additional non-limiting examples of THCAS enzymes may also be found in U.S. Pat. No. 9,512,391 and US Publication No. 2018/0179564, which are incorporated by reference in this application in their entireties.

### Cannabidiolic Acid Synthase (CBDAS)

A host cell described in this application may comprise a TS that is a cannabidiolic acid synthase (CBDAS). As used in this application, a “CBDAS” refers to an enzyme that is capable of catalyzing oxidative cyclization of a prenyl moiety (e.g., terpene) of a compound of Formula (8) to produce a compound of Formula 9. In some embodiments, a compound of Formula 9 is a compound of Formula (9a) (cannabidiolic acid (CBDA)), CBDVA, or CBDP. A CBDAS may use cannabigerolic acid (CBGA) or cannabinerolic acid as a substrate. In some embodiments, a cannabidiolic acid synthase is capable of oxidative cyclization of cannabigerolic acid (CBGA) to produce cannabidiolic acid (CBDA). In some embodiments, the CBDAS may catalyze the oxidative cyclization of other substrates, such as 3-geranyl-2,4-dihydro-6-alkylbenzoic acids like cannabigerovarinic acid (CBVGA). In some embodiments, the CBDAS exhibits specificity for CBGA substrates.

In some embodiments, a CBDAS is from Cannabis. In C. sativa, CBDAS is encoded by the CBDAS gene and is a flavoenzyme. A non-limiting example of an amino acid sequence comprising a CBDAS is provided by UniProtKB—A6P6V9 (SEQ ID NO: 13) from C. sativa in which the signal peptide is underlined and bolded:

MKCSTFSFWFVCKIIFFFFSFNIQTSIANPRENFLKCFSQYIPNNATNLK
LVYTQNNPLYMSVLNSTIHNLRFTSDTTPKPLVIVTPSHVSHIQGTILCS
KKVGLQIRTRSGGHDSEGMSYISQVPFVIVDLRNMRSIKIDVHSQTAWVE
AGATLGEVYYWVNEKNENLSLAAGYCPTVCAGGHFGGGGYGPLMRNYGLA
ADNIIDAHLVNVHGKVLDRKSMGEDLFWALRGGGAESFGIIVAWKIRLVA
VPKSTMFSVKKIMEIHELVKLVNKWQNIAYKYDKDLLLMTHFITRNITDN
QGKNKTAIHTYFSSVFLGGVDSLVDLMNKSFPELGIKKTDCRQLSWIDTI
IFYSGVVNYDTDNFNKEILLDRSAGQNGAFKIKLDYVKKPIPESVFVQIL
EKLYEEDIGAGMYALYPYGGIMDEISESAIPFPHRAGILYELWYICSWEK
QEDNEKHLNWIRNIYNFMTPYVSKNPRLAYLNYRDLDIGINDPKNPNNYT
QARIWGEKYFGKNFDRLVKVKTLVDPNNFFRNEQSIPPLPRHRH

In some embodiments, a CBDAS comprises the sequence shown below:

(SEQ ID NO: 215)
NPRENFLKCFSQYIPNNATNLKLVYTQNNPLYMSVLNSTIHNLRFTSDTT
PKPLVIVTPSHVSHIQGTILCSKKVGLQIRTRSGGHDSEGMSYISQVPFV
IVDLRNMRSIKIDVHSQTAWVEAGATLGEVYYWVNEKNENLSLAAGYCPT
VCAGGHFGGGGYGPLMRNYGLAADNIIDAHLVNVHGKVLDRKSMGEDLFW
ALRGGGAESFGIIVAWKIRLVAVPKSTMFSVKKIMEIHELVKLVNKWQNI
AYKYDKDLLLMTHFITRNITDNQGKNKTAIHTYFSSVFLGGVDSLVDLMN
KSFPELGIKKTDCRQLSWIDTIIFYSGVVNYDTDNFNKEILLDRSAGQNG
AFKIKLDYVKKPIPESVFVQILEKLYEEDIGAGMYALYPYGGIMDEISES
AIPFPHRAGILYELWYICSWEKQEDNEKHLNWIRNIYNFMTPYVSKNPRL
AYLNYRDLDIGINDPKNPNNYTQARIWGEKYFGKNFDRLVKVKTLVDPNN
FFRNEQSIPPLPRHRH

Additional non-limiting examples of CBDAS enzymes may also be found in U.S. Pat. No. 9,512,391 and US Publication No. 2018/0179564, which are incorporated by reference in this application in their entireties.

### Cannabichromenic Acid Synthase (CBCAS)

A host cell described in this application may comprise a TS that is a cannabichromenic acid synthase (CBCAS). As used in this application, a “CBCAS” refers to an enzyme that is capable of catalyzing oxidative cyclization of a prenyl moiety (e.g., terpene) of a compound of Formula (8) to produce a compound of Formula (11). In some embodiments, a compound of Formula (11) is a compound of Formula (11a) (cannabichromenic acid (CBCA)), CBCVA, or a compound of Formula (8) with R as a C7 alkyl (heptyl) group. A CBCAS may use cannabigerolic acid (CBGA) as a substrate. In some embodiments, a CBCAS produces cannabichromenic acid (CBCA) from cannabigerolic acid (CBGA). In some embodiments, the CBCAS may catalyze the oxidative cyclization of other substrates, such as 3-geranyl-2,4-dihydro-6-alkylbenzoic acids like cannabigerovarinic acid (CBVGA), or a substrate of Formula (8) with R as a C7 alkyl (heptyl) group. In some embodiments, the CBCAS exhibits specificity for CBGA substrates.

In some embodiments, a CBCAS is from Cannabis. A C. sativa CBCAS has the amino acid sequence as follows, in which the signal peptide is underlined and bolded:

(SEQ ID NO: 15)
MNCSTFSFWFVCKIIFFFLSFNIQISIANPQENFLKCFSEYIPNNPANPK
FIYTQHDQLYMSVLNSTIQNLRFTSDTTPKPLVIVTPSNVSHIQASILCS
KKVGLQIRTRSGGHDAEGLSYISQVPFAIVDLRNMHTVKVDIHSQTAWVE
AGATLGEVYYWINEMNENFSFPGGYCPTVGVGGHFSGGGYGALMRNYGLA
ADNIIDAHLVNVDGKVLDRKSMGEDLFWAIRGGGGENFGIIAACKIKLVV
VPSKATIFSVKKNMEIHGLVKLFNKWQNIAYKYDKDLMLTTHFRTRNITD
NHGKNKTTVHGYFSSIFLGGVDSLVDLMNKSFPELGIKKTDCKELSWIDT
TIFYSGVVNYNTANFKKEILLDRSAGKKTAFSIKLDYVKKLIPETAMVKI
LEKLYEEEVGVGMYVLYPYGGIMDEISESAIPFPHRAGIMYELWYTATWE
KQEDNEKHINWVRSVYNFTTPYVSQNPRLAYLNYRDLDLGKTNPESPNNY
TQARIWGEKYFGKNFNRLVKVKTKADPNNFFRNEQSIPPLPPRHH.

In some embodiments, a CBCAS comprises the sequence shown below:

(SEQ ID NO: 33)
NPQENFLKCFSEYIPNNPANPKFIYTQHDQLYMSVLNSTIQNLRFTSDTT
PKPLVIVTPSNVSHIQASILCSKKVGLQIRTRSGGHDAEGLSYISQVPFA
IVDLRNMHTVKVDIHSQTAWVEAGATLGEVYYWINEMNENFSFPGGYCPT
VGVGGHFSGGGYGALMRNYGLAADNIIDAHLVNVDGKVLDRKSMGEDLFW
AIRGGGGENFGIIAACKIKLVVVPSKATIFSVKKNMEIHGLVKLFNKWQN
IAYKYDKDLMLTTHFRTRNITDNHGKNKTTVHGYFSSIFLGGVDSLVDLM
NKSFPELGIKKTDCKELSWIDTTIFYSGVVNYNTANFKKEILLDRSAGKK
TAFSIKLDYVKKLIPETAMVKILEKLYEEEVGVGMYVLYPYGGIMDEISE
SAIPFPHRAGIMYELWYTATWEKQEDNEKHINWVRSVYNFTTPYVSQNPR
LAYLNYRDLDLGKTNPESPNNYTQARIWGEKYFGKNFNRLVKVKTKADPN
NFFRNEQSIPPLPPRHH.

In other embodiments, a CBCAS may be a CBCAS described in and incorporated by reference from U.S. Pat. No. 9,359,625.

In some embodiments, a CBCAS may be a C. sativa enzyme that also exhibits THCAS activity, such as a THCAS corresponding to Uniprot KB Accession No.: I1V0C5. In some embodiments, a CBCAS may be a C. sativa THCAS corresponding to any of SEQ ID NOs: 20-24.

As described in the Examples section, it was surprisingly discovered that multiple fungal enzymes, including enzymes of the Aspergillus family, such as an enzyme from A. niger (mold), are capable of catalyzing the conversion of a compound of Formula (8) to produce a compound of Formula (11), and, in some cases, also to produce a compound of Formula (10) and/or a compound of Formula (9). Whereas Cannabis plants have been under artificially high selection pressure to produce cannabinoids through human intervention for centuries, fungal species, such as the A. niger mold, have not been subjected to selection pressure for cannabinoid production. Therefore, without being bound by a particular theory, the fungal CBCASs, such as the A. niger CBCAS, disclosed in this application may be useful for engineering to alter the activity and or abundance of the TS (e.g., change the product profile, substrate profile, and/or kinetics (e.g., Kcat/Vmax and/or Kd) of the TS). It was also surprisingly found, as shown in the Examples section, that many of the fungal enzymes, including enzymes of the Aspergillus family, such as the A. niger enzyme, identified in this disclosure exhibit CBCAS activity, CBCVAS activity, or even both. Some of these enzymes additionally exhibited THCAS activity, THCVAS activity, CBDAS activity, or a combination thereof.

In some embodiments, a CBCAS from A. niger comprises the amino acid sequence shown below:

(SEQ ID NO: 25)
GNTTSIAGRDCLISALGGNSALAVFPNELLWTADVHEYNLNLPVTPAAIT
YPETAAQIAGVVKCASDYDYKVQARSGGHSFGNYGLGGADGAVVVDMKHF
TQFSMDDETYEAVIGPGTTLNDVDIELYNNGKRAMAHGVCPTIKTGGHFT
IGGLGPTARQWGLALDHVEEVEVVLANSSIVRASNTQNQDVFFAVKGAAA
NFGIVTEFKVRTEPAPGLAVQYSYTFNLGSTAEKAQFVKDWQSFISAKNL
TRQFYNNMVIFDGDIILEGLFFGSKEQYDALGLEDHFAPKNPGNILVLTD
WLGMVGHALEDTILKLVGNTPTWFYAKSLGFRQDTLIPSAGIDEFFEYIA
NHTAGTPAWFVTLSLEGGAINDVAEDATAYAHRDVLFWVQLFMVNPVGPI
SDTTYEFTDGLYDVLARAVPESVGHAYLGCPDPRMEDAQQKYWRTNLPRL
QELKEELDPKNTFHHPQGVMPA.

A non-limiting example of a nucleic acid sequence encoding SEQ ID NO: 25 for expression in S. cerevisiae is:

(SEQ ID NO: 26)
ggtaatacgacctctattgccggcagagattgtttgatctcagctttagg
tggtaactccgctcttgcagtttttccaaacgagttgctatggacagctg
acgtacacgaatataatctgaacttgcctgtcactcccgctgctataacc
tacccagaaaccgccgctcagattgccggtgtggttaagtgcgcttctga
ttacgactataaagtccaagcaaggtccggaggtcatagtttcggtaatt
acggcttgggtggagctgacggtgcagttgtcgttgatatgaagcacttc
actcaattttcgatggacgatgaaacttacgaagctgttatcggtccagg
tacaactttaaacgatgtcgacatcgaattgtacaacaacggtaaaagag
ccatggctcatggtgtatgtccaaccattaagactggtggtcacttcacc
atcggtggtctaggacctacggctcgtcaatggggtctggctttggacca
tgtcgaggaagttgaagttgtgttagctaactctagcattgttagagcct
ctaatacacaaaatcaagatgttttctttgcagtcaagggtgctgctgct
aacttcggaatcgtcactgaatttaaagttagaactgaaccagccccagg
tttggctgtacagtactcctataccttcaacttgggttcaactgccgaga
aggctcaattcgttaaggattggcaatctttcatttcggctaagaaccta
accagacaattttataataacatggtcatttttgatggtgacataatctt
ggaaggtttattcttcggtagcaaggaacaatacgacgccttgggccttg
aagatcacttcgcaccaaagaatccaggtaacatattggttttaacagat
tggctaggcatggtgggtcacgcattggaagacactattttaaaattggt
cggtaataccccaacatggttctatgctaagtccttgggttttagacaag
acactctgatcccttctgccggtattgacgaatttttcgaatacattgct
aaccataccgccggcactcctgcttggtttgttactttgtccttagaggg
tggtgctatcaacgatgtcgcagaagatgctacggcctatgctcacagag
atgttttgttctgggtccaactattcatggttaatccagtcggtcctatc
tctgacactacctacgagtttacagacggcttgtacgatgtgttggcccg
tgctgttccagaaagcgtgggacatgcttaccttggttgtccagatccaa
gaatggaagacgctcaacagaagtattggcgtaccaatttgccccgtctg
caagaactaaaggaagagttggatccaaaaaacaccttccatcacccaca
gggtgttatgccagcttaa.

In some embodiments, a CBCAS from A. niger comprises the amino acid sequence shown below (corresponding to UniProt accession no. A0A254UC34):

(SEQ ID NO: 27)
MGNTTSIAGRDCLISALGGNSALAVFPNELLWTADVHEYNLNLPVTPAAI
TYPETAAQIAGVVKCASDYDYKVQARSGGHSFGNYGLGGADGAVVVDMKH
FTQFSMDDETYEAVIGPGTTLNDVDIELYNNGKRAMAHGVCPTIKTGGHF
TIGGLGPTARQWGLALDHVEEVEVVLANSSIVRASNTQNQDVFFAVKGAA
ANFGIVTEFKVRTEPAPGLAVQYSYTFNLGSTAEKAQFVKDWQSFISAKN
LTRQFYNNMVIFDGDIILEGLFFGSKEQYDALGLEDHFAPKNPGNILVLT
DWLGMVGHALEDTILKLVGNTPTWFYAKSLGFRQDTLIPSAGIDEFFEYI
ANHTAGTPAWFVTLSLEGGAINDVAEDATAYAHRDVLFWVQLFMVNPVGP
ISDTTYEFTDGLYDVLARAVPESVGHAYLGCPDPRMEDAQQKYWRTNLPR
LQELKEELDPKNTFHHPQGVMPA.

A non-limiting example of a nucleic acid sequence encoding SEQ ID NO: 27 for expression in S. cerevisiae is:

(SEQ ID NO: 28)
atgggtaatacgacctctattgccggcagagattgtttgatctcagcttt
aggtggtaactccgctcttgcagtttttccaaacgagttgctatggacag
ctgacgtacacgaatataatctgaacttgcctgtcactcccgctgctata
acctacccagaaaccgccgctcagattgccggtgtggttaagtgcgcttc
tgattacgactataaagtccaagcaaggtccggaggtcatagtttcggta
attacggcttgggtggagctgacggtgcagttgtcgttgatatgaagcac
ttcactcaattttcgatggacgatgaaacttacgaagctgttatcggtcc
aggtacaactttaaacgatgtcgacatcgaattgtacaacaacggtaaaa
gagccatggctcatggtgtatgtccaaccattaagactggtggtcacttc
accatcggtggtctaggacctacggctcgtcaatggggtctggctttgga
ccatgtcgaggaagttgaagttgtgttagctaactctagcattgttagag
cctctaatacacaaaatcaagatgttttctttgcagtcaagggtgctgct
gctaacttcggaatcgtcactgaatttaaagttagaactgaaccagcccc
aggtttggctgtacagtactcctataccttcaacttgggttcaactgccg
agaaggctcaattcgttaaggattggcaatctttcatttcggctaagaac
ctaaccagacaattttataataacatggtcatttttgatggtgacataat
cttggaaggtttattcttcggtagcaaggaacaatacgacgccttgggcc
ttgaagatcacttcgcaccaaagaatccaggtaacatattggttttaaca
gattggctaggcatggtgggtcacgcattggaagacactattttaaaatt
ggtcggtaataccccaacatggttctatgctaagtccttgggttttagac
aagacactctgatcccttctgccggtattgacgaatttttcgaatacatt
gctaaccataccgccggcactcctgcttggtttgttactttgtccttaga
gggtggtgctatcaacgatgtcgcagaagatgctacggcctatgctcaca
gagatgttttgttctgggtccaactattcatggttaatccagtcggtcct
atctctgacactacctacgagtttacagacggcttgtacgatgtgttggc
ccgtgctgttccagaaagcgtgggacatgcttaccttggttgtccagatc
caagaatggaagacgctcaacagaagtattggcgtaccaatttgccccgt
ctgcaagaactaaaggaagagttggatccaaaaaacaccttccatcaccc
acagggtgttatgccagcttaa.

In some embodiments, a CBCAS comprises each of: SEQ ID NO: 25; the MFalpha2 signal peptide; and the HDEL signal peptide. In some embodiments, such a CBCAS comprises the amino acid sequence shown below, in which signal peptides are underlined and bolded:

(SEQ ID NO: 29)
MKFISTFLTFILAAVSVTAGNTTSIAGRDCLISALGGNSALAVFPNELLW
TADVHEYNLNLPVTPAAITYPETAAQIAGVVKCASDYDYKVQARSGGHSF
GNYGLGGADGAVVVDMKHFTQFSMDDETYEAVIGPGTTLNDVDIELYNNG
KRAMAHGVCPTIKTGGHFTIGGLGPTARQWGLALDHVEEVEVVLANSSIV
RASNTQNQDVFFAVKGAAANFGIVTEFKVRTEPAPGLAVQYSYTFNLGST
AEKAQFVKDWQSFISAKNLTRQFYNNMVIFDGDIILEGLFFGSKEQYDAL
GLEDHFAPKNPGNILVLTDWLGMVGHALEDTILKLVGNTPTWFYAKSLGF
RQDTLIPSAGIDEFFEYIANHTAGTPAWFVTLSLEGGAINDVAEDATAYA
HRDVLFWVQLFMVNPVGPISDTTYEFTDGLYDVLARAVPESVGHAYLGCP
DPRMEDAQQKYWRTNLPRLQELKEELDPKNTFHHPQGVMPAHDEL.

A non-limiting example of a nucleic acid sequence encoding SEQ ID NO: 29 is shown below, in which sequences encoding signal peptides are underlined and bolded:

(SEQ ID NO: 30)
atgaagtttatcagtaccttcttgacctttatcttggccgctgtctccgt
aaccgctggtaatacgacctctattgccggcagagattgtttgatctcag
ctttaggtggtaactccgctcttgcagtttttccaaacgagttgctatgg
acagctgacgtacacgaatataatctgaacttgcctgtcactcccgctgc
tataacctacccagaaaccgccgctcagattgccggtgtggttaagtgcg
cttctgattacgactataaagtccaagcaaggtccggaggtcatagtttc
ggtaattacggcttgggtggagctgacggtgcagttgtcgttgatatgaa
gcacttcactcaattttcgatggacgatgaaacttacgaagctgttatcg
gtccaggtacaactttaaacgatgtcgacatcgaattgtacaacaacggt
aaaagagccatggctcatggtgtatgtccaaccattaagactggtggtca
cttcaccatcggtggtctaggacctacggctcgtcaatggggtctggctt
tggaccatgtcgaggaagttgaagttgtgttagctaactctagcattgtt
agagcctctaatacacaaaatcaagatgttttctttgcagtcaagggtgc
tgctgctaacttcggaatcgtcactgaatttaaagttagaactgaaccag
ccccaggtttggctgtacagtactcctataccttcaacttgggttcaact
gccgagaaggctcaattcgttaaggattggcaatctttcatttcggctaa
gaacctaaccagacaattttataataacatggtcatttttgatggtgaca
taatcttggaaggtttattcttcggtagcaaggaacaatacgacgccttg
ggccttgaagatcacttcgcaccaaagaatccaggtaacatattggtttt
aacagattggctaggcatggtgggtcacgcattggaagacactattttaa
aattggtcggtaataccccaacatggttctatgctaagtccttgggtttt
agacaagacactctgatcccttctgccggtattgacgaatttttcgaata
cattgctaaccataccgccggcactcctgcttggtttgttactttgtcct
tagagggtggtgctatcaacgatgtcgcagaagatgctacggcctatgct
cacagagatgttttgttctgggtccaactattcatggttaatccagtcgg
tcctatctctgacactacctacgagtttacagacggcttgtacgatgtgt
tggcccgtgctgttccagaaagcgtgggacatgcttaccttggttgtcca
gatccaagaatggaagacgctcaacagaagtattggcgtaccaatttgcc
ccgtctgcaagaactaaaggaagagttggatccaaaaaacaccttccatc
acccacagggtgttatgccagcttaacatgatgaatta.

In some embodiments, a TS comprises a nucleic acid or protein sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to one or more of SEQ ID NOs: 20-30 or 34-173, to any one of the sequences in Table 15, or to any TS disclosed in this application. In some embodiments, a TS comprises a nucleic acid or protein sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to one or more of SEQ ID NOs: 25, 26, 27, 28, 35, 56, 64, 85, 92, 94, 95, 105, 126, 134, 155, 162, 164, and 165. In some embodiments, a TS comprises a nucleic acid or protein sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to one or more of SEQ ID NOs: 25, 26, 27, 28, 35, 42, 56, 60, 64, 105, 85, 92, 94, 95, 112, 126, 130, 134, 155, 162, 164, 165. In some embodiments, a TS comprises a nucleic acid or protein sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to one or more of SEQ ID NOs: 25, 26, 27, 28, 35, 42, 56, 60, 64, 105, 85, 89, 92, 93, 94, 95, 96, 97, 102, 112, 126, 130, 134, 155, 159, 162, 163, 164, 165, 166, 167, and 172.

In some embodiments, a TS comprises a sequence that is at most 5%, at most 10%, at most 15%, at most 20%, at most 25%, at most 30%, at most 35%, at most 40%, at most 45%, at most 50%, at most 55%, at most 60%, at most 65%, at most 70%, at most 71%, at most 72%, at most 73%, at most 74%, at most 75%, at most 76%, at most 77%, at most 78%, at most 79%, at most 80%, at most 81%, at most 82%, at most 83%, at most 84%, at most 85%, at most 86%, at most 87%, at most 88%, at most 89%, at most 90%, at most 91%, at most 92%, at most 93%, at most 94%, at most 95%, at most 96%, at most 97%, at most 98%, at most 99%, or is 100% identical, including all values in between, to one or more of SEQ ID NOs: 20-30 or 34-173, to any one of the sequences in Table 15, or to any TS disclosed in this application. In some embodiments, a TS comprises a sequence that is 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, including all values in between, to one or more of SEQ ID NOs: 20-30 or 34-173, to any one of the sequences in Table 15, or to any TS disclosed in this application.

In some embodiments, a TS sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29 includes a signal peptide that comprises SEQ ID NO: 16 or a sequence that has no more than two amino acid substitutions, insertions, additions, or deletions relative to the sequence of SEQ ID NO: 16. In some embodiments, the signal peptide that comprises SEQ ID NO: 16 or a sequence that has no more than two amino acid substitutions, insertions, additions, or deletions relative to the sequence of SEQ ID NO: 16 is located at the N-terminus of the TS sequence. For example, the signal peptide that comprises SEQ ID NO: 16 or a sequence that has no more than two amino acid substitutions, insertions, additions, or deletions relative to the sequence of SEQ ID NO: 16 may start at position 2 of the TS sequence following a methionine residue.

In some embodiments, a TS sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29 includes a signal peptide that comprises SEQ ID NO: 17 or a sequence that has no more than one amino acid substitution, insertion, addition, or deletion relative to the sequence of SEQ ID NO: 17. In some embodiments, the signal peptide that comprises SEQ ID NO: 17 or a sequence that has no more than one amino acid substitution, insertion, addition, or deletion relative to the sequence of SEQ ID NO: 17 is located at the C-terminus of the sequence that is at least 90% identical to SEQ ID NO: 29.

In some embodiments, a TS comprises a sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NO: 25, 27 or 104-173 wherein the sequence is linked to one or more signal peptides. In some embodiments, a signal peptide that comprises SEQ ID NO: 16 or a sequence that has no more than two amino acid substitutions, insertions, additions, or deletions relative to the sequence of SEQ ID NO: 16 is linked to the N-terminus of the sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NO: 25, 27 or 104-173. In some embodiments, the N-terminal methionine residue of any one of SEQ ID NOs: 27 or 104-173 is not included when the sequence is linked to an N-terminal signal peptide. In some embodiments, a methionine residue is added to the N-terminus of the N-terminal signal peptide (e.g., SEQ ID NO: 16). In some embodiments, a signal peptide that comprises SEQ ID NO: 17 or a sequence that has no more than one amino acid substitution, insertion, addition, or deletion relative to the sequence of SEQ ID NO: 17 is linked to the carboxyl terminus of the sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 25, 27 or 104-173.

In some embodiments, a TS comprises a sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 25, 27, 105, 112, 126, 130, 134, 155, 159, 162, 163, 164, 165, 166, 167, and 172, wherein the sequence is linked to one or more signal peptides. In some embodiments, a signal peptide that comprises SEQ ID NO: 16 or a sequence that has no more than two amino acid substitutions, insertions, additions, or deletions relative to the sequence of SEQ ID NO: 16 is linked to the N-terminus of the sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 25, 27, 105, 112, 126, 130, 134, 155, 159, 162, 163, 164, 165, 166, 167, and 172. In some embodiments, the N-terminal methionine residue of any one of SEQ ID NOs: 27, 105, 112, 126, 130, 134, 155, 159, 162, 163, 164, 165, 166, 167, and 172 is not included when the sequence is linked to an N-terminal signal peptide. In some embodiments, a methionine residue is added to the N-terminus of the N-terminal signal peptide (e.g., SEQ ID NO: 16). In some embodiments, a signal peptide that comprises SEQ ID NO: 17 or a sequence that has no more than one amino acid substitution, insertion, addition, or deletion relative to the sequence of SEQ ID NO: 17 is linked to the carboxyl terminus of the sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 25, 27, 105, 112, 126, 130, 134, 155, 159, 162, 163, 164, 165, 166, 167, and 172.

In some embodiments, relative to SEQ ID NO: 21, a TS comprises an amino acid substitution, deletion, or insertion at a residue corresponding to position 1, 2, 3, 4, 5, 6, 8, 10, 12, 13, 14, 15, 16, 17, 18, 19, 20, 21, 22, 23, 24, 26, 27, 28, 29, 30, 31, 33, 34, 35, 37, 39, 41, 48, 49, 51, 55, 58, 60, 61, 62, 70, 72, 74, 75, 76, 81, 88, 89, 91, 94, 97, 100, 101, 102, 104, 105, 106, 108, 110, 111, 112, 113, 114, 115, 116, 117, 119, 122, 123, 125, 127, 130, 132, 133, 135, 137, 138, 139, 140, 141, 142, 145, 147, 149, 150, 164, 165, 168, 169, 172, 173, 175, 176, 177, 180, 181, 183, 184, 185, 187, 193, 201, 208, 209, 212, 214, 215, 217, 222, 225, 226, 227, 229, 231, 233, 235, 236, 238, 239, 241, 242, 243, 244, 245, 246, 247, 250, 251, 253, 254, 255, 256, 257, 260, 261, 262, 263, 264, 265, 266, 267, 268, 269, 270, 271, 272, 273, 274, 275, 277, 278, 279, 281, 282, 283, 284, 286, 287, 288, 290, 292, 293, 294, 295, 297, 298, 299, 301, 302, 309, 310, 311, 312, 315, 317, 322, 323, 324, 326, 327, 328, 329, 330, 331, 332, 333, 334, 335, 336, 337, 338, 339, 340, 341, 344, 346, 347, 348, 349, 350, 351, 352, 353, 354, 355, 357, 361, 362, 365, 366, 368, 369, 370, 371, 372, 373, 374, 376, 377, 379, 380, 381, 382, 383, 384, 385, 386, 387, 389, 394, 396, 401, 402, 411, 412, 414, 415, 416, 418, 419, 420, 422, 423, 424, 425, 426, 427, 428, 429, 430, 431, 432, 433, 434, 436, 437, 439, 440, 441, 447, 448, 451, 452, 459, 461, 463, 464, 465, 467, 468, 469, 470, 471, 473, 474, 477, 484, 485, 488, 492, 496, 497, 500, 505, 511, 513, 514, 515, 516, and/or 517 in SEQ ID NO: 21. In some embodiments, a TS comprises the amino acid residue that is present in SEQ ID NO: 25 at a position corresponding to position 1, 2, 3, 4, 5, 6, 8, 10, 12, 13, 14, 15, 16, 17, 18, 19, 20, 21, 22, 23, 24, 26, 27, 28, 29, 30, 31, 33, 34, 35, 37, 39, 41, 48, 49, 51, 55, 58, 60, 61, 62, 70, 72, 74, 75, 76, 81, 88, 89, 91, 94, 97, 100, 101, 102, 104, 105, 106, 108, 110, 111, 112, 113, 114, 115, 116, 117, 119, 122, 123, 125, 127, 130, 132, 133, 135, 137, 138, 139, 140, 141, 142, 145, 147, 149, 150, 164, 165, 168, 169, 172, 173, 175, 176, 177, 180, 181, 183, 184, 185, 187, 193, 201, 208, 209, 212, 214, 215, 217, 222, 225, 226, 227, 229, 231, 233, 235, 236, 238, 239, 241, 242, 243, 244, 245, 246, 247, 250, 251, 253, 254, 255, 256, 257, 260, 261, 262, 263, 264, 265, 266, 267, 268, 269, 270, 271, 272, 273, 274, 275, 277, 278, 279, 281, 282, 283, 284, 286, 287, 288, 290, 292, 293, 294, 295, 297, 298, 299, 301, 302, 309, 310, 311, 312, 315, 317, 322, 323, 324, 326, 327, 328, 329, 330, 331, 332, 333, 334, 335, 336, 337, 338, 339, 340, 341, 344, 346, 347, 348, 349, 350, 351, 352, 353, 354, 355, 357, 361, 362, 365, 366, 368, 369, 370, 371, 372, 373, 374, 376, 377, 379, 380, 381, 382, 383, 384, 385, 386, 387, 389, 394, 396, 401, 402, 411, 412, 414, 415, 416, 418, 419, 420, 422, 423, 424, 425, 426, 427, 428, 429, 430, 431, 432, 433, 434, 436, 437, 439, 440, 441, 447, 448, 451, 452, 459, 461, 463, 464, 465, 467, 468, 469, 470, 471, 473, 474, 477, 484, 485, 488, 492, 496, 497, 500, 505, 511, 513, 514, 515, 516, and/or 517 in SEQ ID NO: 21.

Examples 1 and 3 describe the identification of fungal candidate TSs that were surprisingly effective in producing CBCA. Table 14 provides non-limiting examples of sequence motifs that were identified as being enriched in the sequences of candidate TSs that were effective in producing CBCA. In some embodiments, a TS includes one or more of the following motifs, provided in Table 14: KVQARSGGH (SEQ ID NO: 174), RASNTQNQD[VI][FL]FA[VI]K (SEQ ID NO: 176), CPTI[KR]TGGH (SEQ ID NO: 181), WFVTLSLEGGAINDV[AP]EDATAY[AG]H (SEQ ID NO: 184), P[IV]S[DQE]TTY[EDG]F[TA]DGLYDVLA[RQK]AVPES[VA]GHAYLGCPDP[RK]M (SEQ ID NO: 186), MKHF[TNS]QFSM (SEQ ID NO: 189), P[EQ][TS]A[EAD][QE]IA[GA][VI]VKC (SEQ ID NO: 193), RDCL[IV]SA[LV]GGN[SA]A[LH][AV][AV]F[PQ][ND][QE]LL[WY] (SEQ ID NO: 200), RT[EQ][PQ]APGLAVQYSY (SEQ ID NO: 207), and/or WQ[SA]FI[SA][AQ][KE]NLT[RW][QK]FY[NST]NM (SEQ ID NO: 211). In some embodiments, a TS includes the motif KVQARSGGH (SEQ ID NO: 174) at residues corresponding to residues 72-80 in SEQ ID NO: 27.

In some embodiments, a TS includes the motif RASNTQNQD[VI][FL]FA[VI]K (SEQ ID NO: 176) at residues corresponding to residues 183-197 in SEQ ID NO: 27. In some embodiments, the motif

(SEQ ID NO: 176)
RASNTQNQD[VI][FL]FA[VI]K is
(SEQ ID NO: 177)
RASNTQNQDVFFAVK,
(SEQ ID NO: 178)
RASNTQNQDILFAVK,
(SEQ ID NO: 179),
RASNTQNQDILFAIK
or
(SEQ ID NO: 180)
RASNTQNQDVLFAVK.

In some embodiments, a TS includes the motif CPTI[KR]TGGH (SEQ ID NO: 181) at residues corresponding to residues 141-149 in SEQ ID NO: 27. In some embodiments, the motif CPTI[KR]TGGH (SEQ ID NO: 181) is CPTIKTGGH (SEQ ID NO: 182) or CPTIRTGGH (SEQ ID NO: 183).

In some embodiments, a TS includes the motif WFVTLSLEGGAINDV[AP]EDATAY[AG]H (SEQ ID NO: 184) at residues corresponding to residues 360-383 in SEQ ID NO: 27. In some embodiments, the motif

(SEQ ID NO: 184)
WFVTLSLEGGAINDV[AP]EDATAY[AG]H is
(SEQ ID NO: 185)
WFVTLSLEGGAINDVAEDATAYAH.

In some embodiments, a TS includes the motif P[IV]S[DQE]TTY[EDG]F[TA]DGLYDVLA[RQK]AVPES[VA]GHAYLGCPDP[RK]M (SEQ ID NO: 186) at residues corresponding to residues 400-436 in SEQ ID NO: 27. In some embodiments, the motif

(SEQ ID NO: 186)
P[IV]S[DQE]TTY[EDG]F[TA]DGLYDVLA[RQK]AVPES[VA]GHAY
LGCPDP[RK]M is
(SEQ ID NO: 187)
PISDTTYEFTDGLYDVLARAVPESVGHAYLGCPDPRM
or
(SEQ ID NO: 188)
PISETTYEFTDGLYDVLARAVPESVGHAYLGCPDPRM.

In some embodiments, a TS includes the motif MKHF[TNS]QFSM (SEQ ID NO: 189) at residues corresponding to residues 98-106 in SEQ ID NO: 27. In some embodiments, the motif MKHF[TNS]QFSM (SEQ ID NO: 189) is MKHFTQFSM (SEQ ID NO: 190), MKHFSQFSM (SEQ ID NO: 191), or MKHFNQFSM (SEQ ID NO: 192).

In some embodiments, a TS includes the motif P[EQ][TS]A[EAD][QE]IA[GA][VI]VKC (SEQ ID NO: 193) at residues corresponding to residues 53-65 in SEQ ID NO: 27. In some embodiments, the motif

(SEQ ID NO: 193)
P[EQ][TS]A[EAD][QE]IA[GA][VI]VKC is
(SEQ ID NO: 194),
PETAEQIAGIVKC,
(SEQ ID NO: 195)
PQSADEIAAVVKC,
(SEQ ID NO: 196)
PETAAQIAGVVKC,
(SEQ ID NO: 197)
PQSAEEIAAVVKC,
(SEQ ID NO: 198)
PETAEQIAGVVKC,
or
(SEQ ID NO: 199)
PETAEQIAAVVKC.

In some embodiments, a TS includes the motif

(SEQ ID NO: 200)
RDCL[IV]SA[LV]GGN[SA]A[LH][AV][AV]F[PQ][ND][QE]LL
[WY]

at residues corresponding to residues 10-32 in SEQ ID NO: 27. In some embodiments, the motif RDCL[IV]SA[LV]GGN[SA]A[LH][AV][AV]F[PQ][ND][QE]LL[WY] (SEQ ID NO: 200) is RDCLISAVGGNAAHVAFQDQLLY (SEQ ID NO: 201), RDCLISALGGNSALAVFPNELLW (SEQ ID NO: 202), RDCLISALGGNSALAAFPNELLW (SEQ ID NO: 203), RDCLISALGGNSALAVFPNQLLW (SEQ ID NO: 204), RDCLISALGGNSALAAFPNQLLW (SEQ ID NO: 205), or RDCLVSALGGNSALAAFPNQLLW (SEQ ID NO: 206).

In some embodiments, a TS includes the motif RT[EQ][PQ]APGLAVQYSY (SEQ ID NO: 207) at residues corresponding to residues 212-225 in SEQ ID NO: 27. In some embodiments, the motif RT[EQ][PQ]APGLAVQYSY (SEQ ID NO: 207) is RTEPAPGLAVQYSY (SEQ ID NO: 208), RTEQAPGLAVQYSY (SEQ ID NO: 209), or RTQPAPGLAVQYSY (SEQ ID NO: 210).

In some embodiments, a TS includes the motif WQ[SA]FI[SA][AQ][KE]NLT[RW][QK]FY[NST]NM (SEQ ID NO: 211) at residues corresponding to residues 242-259 in SEQ ID NO: 27. In some embodiments, the motif

(SEQ ID NO: 211)
WQ[SA]FI[SA][AQ][KE]NLT[RW][QK]FY[NST]NM is
(SEQ ID NO: 212)
WQSFISAKNLTRQFYNNM
or
(SEQ ID NO: 213)
WQSFISAKNLTRQFYTNM.

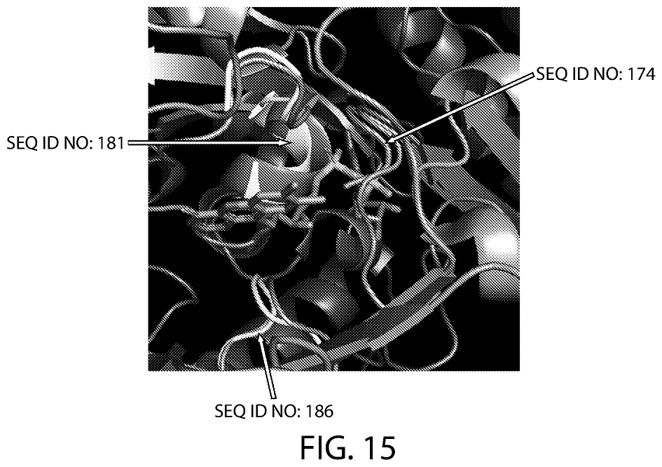

In some embodiments, one or more of the motifs described above may contact the cofactor (FAD) binding site of the TS. For example, KVQARSGGH (SEQ ID NO: 174), CPTI[KR]TGGH (SEQ ID NO: 181), and P[IV]S[DQE]TTY[EDG]F[TA]DGLYDVLA[RQK]AVPES[VA]GHAYLGCPDP[RK]M (SEQ ID NO: 186), indicated by arrows in FIG. 15, are predicted to contact the cofactor binding site and may therefore influence cofactor binding. Without wishing to be bound by any theory, these motifs may be involved in modulating the redox potential of the cofactor and may be important for enzyme activity by regulating, for example, enzyme turnover.

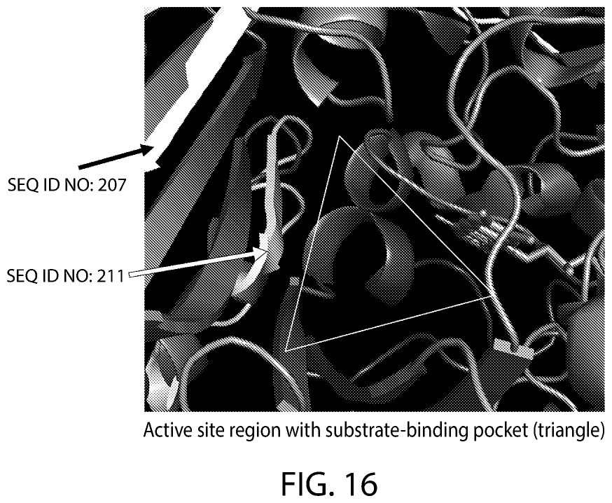

In some embodiments, one or more of the motifs described above may line the cavity of the active site of the TS. For example, WQ[SA]FI[SA][AQ][KE]NLT[RW][QK]FY[NST]NM (SEQ ID NO: 211), indicated by an arrow in FIG. 16, is predicted to line the cavity of the active site. In some embodiments, motifs RT[EQ][PQ]APGLAVQYSY (SEQ ID NO: 207) and WFVTLSLEGGAINDV[AP]EDATAY[AG]H (SEQ ID NO: 184) may also line the cavity of the active site and be near the substrate binding pocket. Without wishing to be bound by any theory, these motifs may influence substrate or product specificity.

In some embodiments, a TS associated with this disclosure comprises one or more amino acid substitutions, deletions, additions, or insertions relative to the sequence of any of the TSs provided in this disclosure. In some embodiments, relative to the sequence of SEQ ID NO: 27, the TS comprises an amino acid substitution at a residue corresponding to position 25, 33, 35, 39, 43, 55, 57, 61, 62, 63, 71, 102, 112, 114, 122, 126, 129, 131, 161, 180, 183, 202, 256, 257, 260, 262, 280, 287, 295, 341, 353, 386, 392, 394, 398, 410, 423, 426, 446, 450, 456, 458, 466, 469, and/or 472 in SEQ ID NO: 27. In some embodiments, relative to the sequence of SEQ ID NO: 27, the TS comprises an amino acid substitution at a residue corresponding to position 33, 39, 55, 57, 61, 62, 63, 71, 112, 122, 126, 129, 131 180, 183, 202, 256, 257, 260, 287, 295, 341, 386, 392, 394, 398, 410, 423, 426, 450, and/or 472.

In some embodiments, the TS comprises: the amino acid A at a residue corresponding to position 25 in SEQ ID NO: 27; the amino acid D at a residue corresponding to position 33 in SEQ ID NO: 27; the amino acid A at a residue corresponding to position 35 in SEQ ID NO: 27; the amino acid F at a residue corresponding to position 39 in SEQ ID NO: 27; the amino acid I at a residue corresponding to position 43 in SEQ ID NO: 27; the amino acid S at a residue corresponding to position 55 in SEQ ID NO: 27; the amino acid Q at a residue corresponding to position 57 in SEQ ID NO: 27; the amino acid E at a residue corresponding to position 57 in SEQ ID NO: 27; the amino acid A at a residue corresponding to position 61 in SEQ ID NO: 27; the amino acid I at a residue corresponding to position 62 in SEQ ID NO: 27; the amino acid I at a residue corresponding to position 63 in SEQ ID NO: 27; the amino acid I at a residue corresponding to position 71 in SEQ ID NO: 27; the amino acid N at a residue corresponding to position 102 in SEQ ID NO: 27; the amino acid Q at a residue corresponding to position 102 in SEQ ID NO: 27; the amino acid S at a residue corresponding to position 102 in SEQ ID NO: 27; the amino acid V at a residue corresponding to position 112 in SEQ ID NO: 27; the amino acid T at a residue corresponding to position 112 in SEQ ID NO: 27; the amino acid T at a residue corresponding to position 114 in SEQ ID NO: 27; the amino acid S at a residue corresponding to position 122 in SEQ ID NO: 27; the amino acid G at a residue corresponding to position 122 in SEQ ID NO: 27; the amino acid A at a residue corresponding to position 122 in SEQ ID NO: 27; the amino acid E at a residue corresponding to position 122 in SEQ ID NO: 27; the amino acid A at a residue corresponding to position 126 in SEQ ID NO: 27; the amino acid R at a residue corresponding to position 126 in SEQ ID NO: 27; the amino acid T at a residue corresponding to position 126 in SEQ ID NO: 27; the amino acid K at a residue corresponding to position 126 in SEQ ID NO: 27; the amino acid D at a residue corresponding to position 126 in SEQ ID NO: 27; the amino acid W at a residue corresponding to position 129 in SEQ ID NO: 27; the amino acid S at a residue corresponding to position 131 in SEQ ID NO: 27; the amino acid K at a residue corresponding to position 161 in SEQ ID NO: 27; the amino acid T at a residue corresponding to position 180 in SEQ ID NO: 27; the amino acid T at a residue corresponding to position 183 in SEQ ID NO: 27; the amino acid S at a residue corresponding to position 202 in SEQ ID NO: 27; the amino acid G at a residue corresponding to position 202 in SEQ ID NO: 27; the amino acid S at a residue corresponding to position 202 in SEQ ID NO: 27; the amino acid F at a residue corresponding to position 256 in SEQ ID NO: 27; the amino acid M at a residue corresponding to position 256 in SEQ ID NO: 27; the amino acid S at a residue corresponding to position 257 in SEQ ID NO: 27; the amino acid M at a residue corresponding to position 260 in SEQ ID NO: 27; the amino acid F at a residue corresponding to position 260 in SEQ ID NO: 27; the amino acid I at a residue corresponding to position 262 in SEQ ID NO: 27; the amino acid N at a residue corresponding to position 280 in SEQ ID NO: 27; the amino acid R at a residue corresponding to position 287 in SEQ ID NO: 27; the amino acid S at a residue corresponding to position 295 in SEQ ID NO: 27; the amino acid S at a residue corresponding to position 341 in SEQ ID NO: 27; the amino acid A at a residue corresponding to position 353 in SEQ ID NO: 27; the amino acid A at a residue corresponding to position 386 in SEQ ID NO: 27; the amino acid H at a residue corresponding to position 392 in SEQ ID NO: 27; the amino acid T at a residue corresponding to position 394 in SEQ ID NO: 27; the amino acid F at a residue corresponding to position 398 in SEQ ID NO: 27; the amino acid T at a residue corresponding to position 398 in SEQ ID NO: 27; the amino acid A at a residue corresponding to position 398 in SEQ ID NO: 27; the amino acid L at a residue corresponding to position 398 in SEQ ID NO: 27; the amino acid N at a residue corresponding to position 410 in SEQ ID NO: 27; the amino acid A at a residue corresponding to position 423 in SEQ ID NO: 27; the amino acid Y at a residue corresponding to position 426 in SEQ ID NO: 27; the amino acid P at a residue corresponding to position 446 in SEQ ID NO: 27; the amino acid K at a residue corresponding to position 450 in SEQ ID NO: 27; the amino acid A at a residue corresponding to position 456 in SEQ ID NO: 27; the amino acid W at a residue corresponding to position 458 in SEQ ID NO: 27; the amino acid N at a residue corresponding to position 466 in SEQ ID NO: 27; the amino acid S at a residue corresponding to position 469 in SEQ ID NO: 27; the amino acid R at a residue corresponding to position 472 in SEQ ID NO: 27; the amino acid A at a residue corresponding to position 472 in SEQ ID NO: 27; and/or the amino acid K at a residue corresponding to position 450 in SEQ ID NO: 27.

In some embodiments, the TS comprises one or more of the following amino acid substitutions relative to SEQ ID NO: 27: V25A; T33D; D35A Y39F; L43I; T55S; A57Q; A57E; G61A; V62I; V63I; Y71I; T102N; T102Q; T102S; E112V; E112T; V114T; N122S; N122G; N122A; N122E; I126A; I126R; I126T; I126K; I126D; Y129W; N131S; Q161K; S180T; R183T; N202S; N202G; Y256F; Y256M; N257S; V260M; V260F; F262I; D280N; H287R; N295S; A341S; H353A; V386A; L392H; M394T; V398F; V398T; V398A; V398L; D410N; S423A; H426Y; T446P; R450K; E456A; L458W; H466N; G469S; P472R; P472A; and/or R450K.

Residues Y256, L392, and M394 of SEQ ID NO: 27, which are all large, hydrophobic amino acids, are predicted to be located within the active site. Without wishing to be bound by any theory, mutations at these positions may shift the product profile toward CBCA and away from CBDA at least in part by physically blocking the folding of CBGA in a manner that sterically prevents CBDA synthesis.

In some embodiments, one or more amino acid substitutions increases the product specificity of the TS, such as the specificity for a compound of Formula (11), CBCA, CBCVA or a combination thereof, as compared to a TS without such substitution. In some embodiments, the one or more amino acid substitutions include: A57Q and G61A; V260M; V62I; V386A; V260F; E112V and N122S; A57E and I126A; T33D and N257S; N202S and P472A; D410N; R450K; S180T; R183T; N122G and I126R; N122A and I126T; Y71I; H287R and A341S; T55S and I126T; N122G and V398F; M394T; A57E; N131S; V63I; N122G and I126R; P472R; S180T; V398A; R183T; V260M; V386A; H426Y; Y256M; N202S and P472A; N122G and I126K; V62I; R450K; Y129W; S423A; H287R and A341S; N295S; Y39F; V260F; L392H; A57E and N131S; E112V and N122S; T33D and N257S.

In some embodiments, the one or more amino acid substitutions include: A57Q and G61A; Y71I; and/or V260F.

TABLE 3
Mutations in A. niger CBCAS that
demonstrated increased CBCA titer

| | |
|---|---|
| Residue in | |
| SEQ ID NO: 27 | Amino Acid Substitutions |

| | | | | |
|---|---|---|---|---|
| T33 | D | — | — | — |
| Y39 | F | — | — | — |
| T55 | S | — | — | — |
| A57 | Q | E | — | — |
| G61 | A | — | — | — |
| V62 | I | — | — | — |
| V63 | I | — | — | — |
| Y71 | I | — | — | — |
| E112 | V | — | — | — |
| N122 | S | G | A | — |
| I126 | A | R | T | K |
| Y129 | W | — | — | — |
| N131 | S | — | — | — |
| S180 | T | — | — | — |
| R183 | T | — | — | — |
| N202 | S | — | — | — |
| Y256 | M | — | — | — |
| N257 | S | — | — | — |
| V260 | M | F | — | — |
| H287 | R | — | — | — |
| N295 | S | — | — | — |
| A341 | S | — | — | — |
| V386 | A | — | — | — |
| L392 | H | — | — | — |
| M394 | T | — | — | — |
| V398 | F | A | — | — |
| D410 | N | — | — | — |
| S423 | A | — | — | — |
| H426 | Y | — | — | — |
| R450 | K | — | — | — |
| P472 | A | R | — | — |

### Additional Cannabinoid Pathway Enzymes

Methods for production of cannabinoids and cannabinoid precursors can further include expression of one or more of: an acyl activating anzyme (AAE); a polyketide synthase (PKS) (e.g., OLS); a polykeide cyclase (PKC); and a prenyltransferase (PT).

### Acyl Activating Enzyme (AAE)

A host cell described in this disclosure may comprise an AAE. As used in this disclosure, an AAE refers to an enzyme that is capable of catalyzing the esterification between a thiol and a substrate (e.g., optionally substituted aliphatic or aryl group) that has a carboxylic acid moiety. In some embodiments, an AAE is capable of using Formula (1):

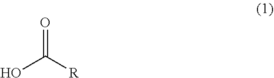

or a salt, solvate, hydrate, polymorph, co-crystal, tautomer, stereoisomer, isotopically labeled derivative thereof to produce a product of Formula (2):

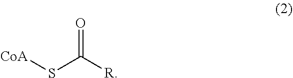

R is as defined in this application. In certain embodiments, R is hydrogen. In certain embodiments, R is optionally substituted alkyl. In certain embodiments, R is optionally substituted C1-40 alkyl. In certain embodiments, R is optionally substituted C2-40 alkyl. In certain embodiments, R is optionally substituted C2-40 alkyl, which is straight chain or branched alkyl. In certain embodiments, R is optionally substituted C2-10 alkyl, optionally substituted C10-C20 alkyl, optionally substituted C20-C30 alkyl, optionally substituted C30-C40 alkyl, or optionally substituted C40-C50 alkyl, which is straight chain or branched alkyl. In certain embodiments, R is optionally substituted C3-8 alkyl. In certain embodiments, R is optionally substituted C1-C40 alkyl, C1-C20 alkyl, C1-C10 alkyl, C1-C8 alkyl, C1-C5 alkyl, C3-C5 alkyl, C3 alkyl, or C5 alkyl. In certain embodiments, R is optionally substituted C1-C20 alkyl. In certain embodiments, R is optionally substituted C1-C20 branched alkyl. In certain embodiments, R is optionally substituted C1-C20 alkyl, optionally substituted C1-C10 alkyl, optionally substituted C10-C20 alkyl, optionally substituted C20-C30 alkyl, optionally substituted C30-C40 alkyl, or optionally substituted C40-C50 alkyl. In certain embodiments, R is optionally substituted C1-C10 alkyl. In certain embodiments, R is optionally substituted C3 alkyl. In certain embodiments, R is optionally substituted n-propyl. In certain embodiments, R is unsubstituted n-propyl. In certain embodiments, R is optionally substituted C1-C8 alkyl. In some embodiments, R is a C2-C6 alkyl. In certain embodiments, R is optionally substituted C1-C5 alkyl. In certain embodiments, R is optionally substituted C3-C5 alkyl. In certain embodiments, R is optionally substituted C3 alkyl. In certain embodiments, R is optionally substituted C5 alkyl. In certain embodiments, R is of formula:

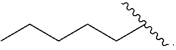 [SMILES: *CCCCC]

In certain embodiments, R is of formula:

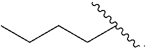 [SMILES: *CCCC]

In certain embodiments, R is of formula:

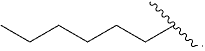 [SMILES: *CCCCCC]

In certain embodiments, R is of formula:

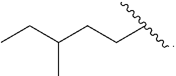 [SMILES: *CCC(C)CC]

In certain embodiments, R is optionally substituted propyl. In certain embodiments, R is optionally substituted n-propyl. In certain embodiments, R is n-propyl optionally substituted with optionally substituted aryl. In certain embodiments, R is n-propyl optionally substituted with optionally substituted phenyl. In certain embodiments, R is n-propyl substituted with unsubstituted phenyl. In certain embodiments, R is optionally substituted butyl. In certain embodiments, R is optionally substituted n-butyl. In certain embodiments, R is n-butyl optionally substituted with optionally substituted aryl. In certain embodiments, R is n-butyl optionally substituted with optionally substituted phenyl. In certain embodiments, R is n-butyl substituted with unsubstituted phenyl. In certain embodiments, R is optionally substituted pentyl. In certain embodiments, R is optionally substituted n-pentyl. In certain embodiments, R is n-pentyl optionally substituted with optionally substituted aryl. In certain embodiments, R is n-pentyl optionally substituted with optionally substituted phenyl. In certain embodiments, R is n-pentyl substituted with unsubstituted phenyl. In certain embodiments, R is optionally substituted hexyl. In certain embodiments, R is optionally substituted n-hexyl. In certain embodiments, R is optionally substituted n-heptyl. In certain embodiments, R is optionally substituted n-octyl. In certain embodiments, R is alkyl optionally substituted with aryl (e.g., phenyl). In certain embodiments, R is optionally substituted acyl (e.g., —C(═O)Me).

In certain embodiments, R is optionally substituted alkenyl (e.g., substituted or unsubstituted C₂₋₆ alkenyl). In certain embodiments, R is substituted or unsubstituted C₂₋₆ alkenyl. In certain embodiments, R is substituted or unsubstituted C₂₋₅ alkenyl. In certain embodiments, R is of formula:

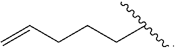 [SMILES: *CCCC=C]

In certain embodiments, R is optionally substituted alkynyl (e.g., substituted or unsubstituted C₂₋₆ alkynyl). In certain embodiments, R is substituted or unsubstituted C₂₋₆ alkynyl. In certain embodiments, R is of formula:

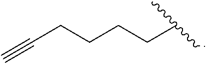 [SMILES: *CCCCC#C]

In certain embodiments, R is optionally substituted carbocyclyl. In certain embodiments, R is optionally substituted aryl (e.g., phenyl or napthyl).

In some embodiments, a substrate for an AAE is produced by fatty acid metabolism within a host cell. In some embodiments, a substrate for an AAE is provided exogenously.

In some embodiments, an AAE is capable of catalyzing the formation of hexanoyl-coenzyme A (hexanoyl-CoA) from hexanoic acid and coenzyme A (CoA). In some embodiments, an AAE is capable of catalyzing the formation of butanoyl-coenzyme A (butanoyl-CoA) from butanoic acid and coenzyme A (CoA).

As one of ordinary skill in the art would appreciate, an AAE could be obtained from any source, including naturally occurring sources and synthetic sources (e.g., a non-naturally occurring AAE). In some embodiments, an AAE is a Cannabis enzyme. Non-limiting examples of AAEs include C. sativa hexanoyl-CoA synthetase 1 (CsHCS1) and C. sativa hexanoyl-CoA synthetase 2 (CsHCS2) as disclosed in U.S. Pat. No. 9,546,362, which is incorporated by reference in this application in its entirety.

CsHCS1 has the sequence:

(SEQ ID NO: 5)
MGKNYKSLDSVVASDFIALGITSEVAETLHGRLAEIVCNYGAATPQTWIN
IANHILSPDLPFSLHQMLFYGCYKDFGPAPPAWIPDPEKVKSTNLGALLE
KRGKEFLGVKYKDPISSFSHFQEFSVRNPEVYWRTVLMDEMKISFSKDPE
CILRRDDINNPGGSEWLPGGYLNSAKNCLNVNSNKKLNDTMIVWRDEGND
DLPLNKLTLDQLRKRVWLVGYALEEMGLEKGCAIAIDMPMHVDAVVIYLA
IVLAGYVVVSIADSFSAPEISTRLRLSKAKAIFTQDHIIRGKKRIPLYSR
VVEAKSPMAIVIPCSGSNIGAELRDGDISWDYFLERAKEFKNCEFTAREQ
PVDAYTNILFSSGTTGEPKAIPWTQATPLKAAADGWSHLDIRKGDVIVWP
TNLGWMMGPWLVYASLLNGASIALYNGSPLVSGFAKFVQDAKVTMLGVVP
SIVRSWKSTNCVSGYDWSTIRCFSSSGEASNVDEYLWLMGRANYKPVIEM
CGGTEIGGAFSAGSFLQAQSLSSFSSQCMGCTLYILDKNGYPMPKNKPGI
GELALGPVMFGASKTLLNGNHHDVYFKGMPTLNGEVLRRHGDIFELTSNG
YYHAHGRADDTMNIGGIKISSIEIERVCNEVDDRVFETTAIGVPPLGGGP
EQLVIFFVLKDSNDTTIDLNQLRLSFNLGLQKKLNPLFKVTRVVPLSSLP
RTATNKIMRRVLRQFSHFE.

CsHCS2 has the sequence:

(SEQ ID NO: 6)
MEKSGYGRDGIYRSLRPPLHLPNNNNLSMVSFLFRNSSSYPQKPALIDSE
TNQILSFSHFKSTVIKVSHGFLNLGIKKNDVVLIYAPNSIHFPVCFLGII
ASGAIATTSNPLYTVSELSKQVKDSNPKLIITVPQLLEKVKGFNLPTILI
GPDSEQESSSDKVMTFNDLVNLGGSSGSEFPIVDDFKQSDTAALLYSSGT
TGMSKGVVLTHKNFIASSLMVTMEQDLVGEMDNVFLCFLPMFHVFGLAII
TYAQLQRGNTVISMARFDLEKMLKDVEKYKVTHLWVVPPVILALSKNSMV
KKFNLSSIKYIGSGAAPLGKDLMEECSKVVPYGIVAQGYGMTETCGIVSM
EDIRGGKRNSGSAGMLASGVEAQIVSVDTLKPLPPNQLGEIWVKGPNMMQ
GYFNNPQATKLTIDKKGWVHTGDLGYFDEDGHLYVVDRIKELIKYKGFQV
APAELEGLLVSHPEILDAVVIPFPDAEAGEVPVAYVVRSPNSSLTENDVK
KFIAGQVASFKRLRKVTFINSVPKSASGKILRRELIQKVRSNM.

### Polyketide Synthases (PKS)

A host cell described in this application may comprise a PKS. As used in this application, a “PKS” refers to an enzyme that is capable of producing a polyketide. In certain embodiments, a PKS converts a compound of Formula (2) to a compound of Formula (4), (5), and/or (6). In certain embodiments, a PKS converts a compound of Formula (2) to a compound of Formula (4). In certain embodiments, a PKS converts a compound of Formula (2) to a compound of Formula (5). In certain embodiments, a PKS converts a compound of Formula (2) to a compound of Formula (4) and/or (5). In certain embodiments, a PKS converts a compound of Formula (2) to a compound of Formula (5) and/or (6).

In some embodiments, a PKS is a tetraketide synthase (TKS). In certain embodiments, a PKS is an olivetol synthase (OLS). As used in this application, an “OLS” refers to an enzyme that is capable of using a substrate of Formula (2a) to form a compound of Formula (4a), (5a) or (6a) as shown in FIG. 1.

In certain embodiments, a PKS is a divarinic acid synthase (DVS).

In certain embodiments, polyketide synthases can use hexanoyl-CoA or any acyl-CoA (or a product of Formula (2):

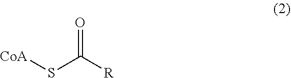

and three malonyl-CoAs as substrates to form 3,5,7-trioxododecanoyl-CoA or other 3,5,7-trioxo-acyl-CoA derivatives; or to form a compound of Formula (4):

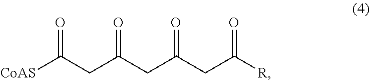

wherein R is hydrogen, optionally substituted acyl, optionally substituted alkyl, optionally substituted alkenyl, optionally substituted alkynyl, optionally substituted carbocyclyl, or optionally substituted aryl; depending on substrate. R is as defined in this application. In some embodiments, R is a C2-C6 optionally substituted alkyl. In some embodiments, R is a propyl or pentyl. In some embodiments, R is pentyl. In some embodiments, R is propyl. A PKS may also bind isovaleryl-CoA, octanoyl-CoA, hexanoyl-CoA, and butyryl-CoA. In some embodiments, a PKS is capable of catalyzing the formation of a 3,5,7-trioxoalkanoyl-CoA (e.g. 3,5,7-trioxododecanoyl-CoA). In some embodiments, an OLS is capable of catalyzing the formation of a 3,5,7-trioxoalkanoyl-CoA (e.g. 3,5,7-trioxododecanoyl-CoA).

In some embodiments, a PKS uses a substrate of Formula (2) to form a compound of Formula (4):

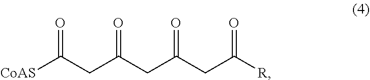

wherein R is unsubstituted pentyl.

As one of ordinary skill in the art would appreciate a PKS, such as an OLS, could be obtained from any source, including naturally occurring sources and synthetic sources (e.g., a non-naturally occurring PKS). In some embodiments a PKS is from Cannabis. In some embodiments a PKS is from Dictyostelium. Non-limiting examples of PKS enzymes may be found in U.S. Pat. No. 6,265,633; WO 2018/148848 A1; WO 2018/148849 A1; and US 2018/155748, which are incorporated by reference in this application in their entireties.

A non-limiting example of an OLS is provided by UniProtKB—B1Q2B6 from C. sativa. In C. sativa, this OLS uses hexanoyl-CoA and malonyl-CoA as substrates to form 3,5,7-trioxododecanoyl-CoA. OLS (e.g., UniProtKB—B1Q2B6) in combination with olivetolic acid cyclase (OAC) produces olivetolic acid (OA) in C. sativa.

The amino acid sequence of UniProtKB—B1Q2B6 is:

(SEQ ID NO: 7)
MNHLRAEGPASVLAIGTANPENILLQDEFPDYYFRVTKSEHMTQLKEKFR
KICDKSMIRKRNCFLNEEHLKQNPRLVEHEMQTLDARQDMLVVEVPKLGK
DACAKAIKEWGQPKSKITHLIFTSASTTDMPGADYHCAKLLGLSPSVKRV
MMYQLGCYGGGTVLRIAKDIAENNKGARVLAVCCDIMACLFRGPSESDLE
LLVGQAIFGDGAAAVIVGAEPDESVGERPIFELVSTGQTILPNSEGTIGG
HIREAGLIFDLHKDVPMLISNNIEKCLIEAFTPIGISDWNSIFWITHPGG
KAILDKVEEKLHLKSDKFVDSRHVLSEHGNMSSSTVLFVMDELRKRSLEE
GKSTTGDGFEWGVLFGFGPGLTVERVVVRSVPIKY.

PKS enzymes described in this application may or may not have cyclase activity. In some embodiments where the PKS enzyme does not have cyclase activity, one or more exogenous polynucleotides that encode a polyketide cyclase (PKC) enzyme may also be co-expressed in the same host cells to enable conversion of hexanoic acid or butyric acid or other fatty acid conversion into olivetolic acid or divarinolic acid or other precursors of cannabinoids. In some embodiments, the PKS enzyme and a PKC enzyme are expressed as separate distinct enzymes. In some embodiments, a PKS enzyme that lacks cyclase activity and a PKC are linked as part of a fusion polypeptide that is a bifunctional PKS. In some embodiments, a bifunctional PKC is referred to as a bifunctional PKS-PKC. In some embodiments, a bifunctional PKC is a bifunctional tetraketide synthase (TKS-TKC). As used in this application, a bifunctional PKS is an enzyme that is capable of producing a compound of Formula (6):

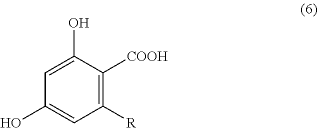

from a compound of Formula (2):

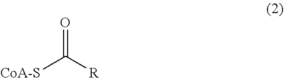

and a compound of Formula (3):

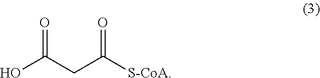

In some embodiments, a PKS produces more of a compound of Formula (6):

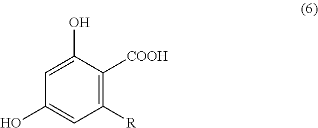

as compared to a compound of Formula (5):

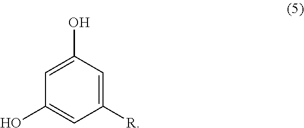

As a non-limiting example, a compound of Formula (6):

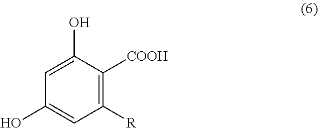

is olivetolic acid (Formula (6a)):

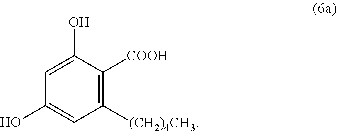

As a non-limiting example, a compound of Formula (5):

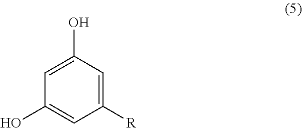

is olivetol (Formula (5a)):

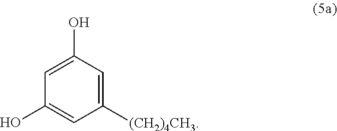

In some embodiments, a polyketide synthase of the present disclosure is capable of catalyzing a compound of Formula (2):

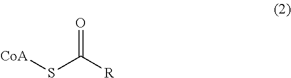

and a compound of Formula (3):

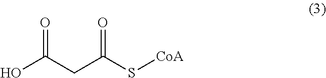

to produce a compound of Formula (4):

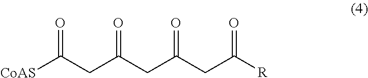

and also further catalyzes a compound of Formula (4):

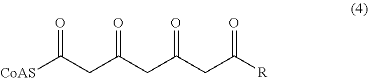

to produce a compound of Formula (6):

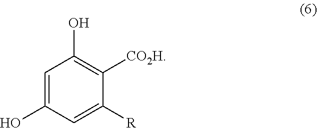

In some embodiments, the PKS is not a fusion protein. In some embodiments, a PKS that is capable of catalyzing a compound of Formula (2):

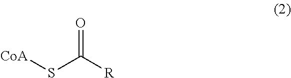

and a compound of Formula (3):

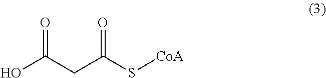

to produce a compound of Formula (4):

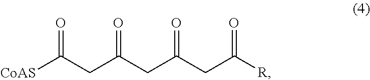

and is also capable of further catalyzing the production of a compound of Formula (6):

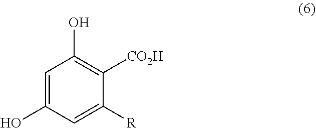

from the compound of Formula (4):

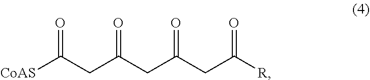

is preferred because it avoids the need for an additional polyketide cyclase to produce a compound of Formula (6):

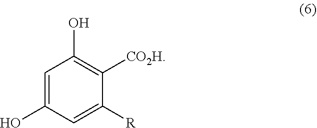

In some embodiments, such an enzyme that is a bifunctional PKS eliminates the transport considerations needed with addition of a polyketide cyclase, whereby the compound of Formula (4), being the product of the PKS, must be transported to the PKS for use as a substrate to be converted into the compound of Formula (6).

In some embodiments, a PKS is capable of producing olivetolic acid in the presence of a compound of Formula (2a):

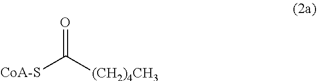

### and Formula (3a):

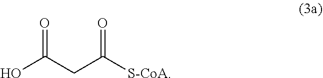

In some embodiments, an OLS is capable of producing olivetolic acid in the presence of a compound of Formula (2a):

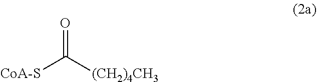

### and Formula (3a):

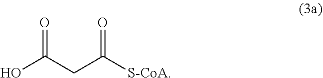

### Polyketide Cyclase (PKC)

A host cell described in this disclosure may comprise a PKC. As used in this application, a “PKC” refers to an enzyme that is capable of cyclizing a polyketide.

In certain embodiments, a polyketide cyclase (PKC) catalyzes the cyclization of an oxo fatty acyl-CoA (e.g., a compound of Formula (4):

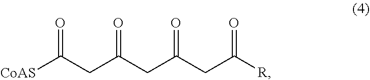

or 3,5,7-trioxododecanoyl-COA, 3,5,7-trioxodecanoyl-COA) to the corresponding intramolecular cyclization product (e.g., compound of Formula (6), including olivetolic acid and divarinic acid). In some embodiments, a PKC catalyzes the formation of a compound which occurs in the presence of a PKS. PKC substrates include trioxoalkanol-CoA, such as 3,5,7-Trioxododecanoyl-CoA, or a compound of Formula (4):

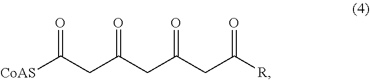

wherein R is hydrogen, optionally substituted acyl, optionally substituted alkyl, optionally substituted alkenyl, optionally substituted alkynyl, optionally substituted carbocyclyl, or optionally substituted aryl. In certain embodiments, a PKC catalyzes a compound of Formula (4):

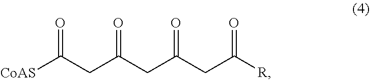

wherein R is hydrogen, optionally substituted acyl, optionally substituted alkyl, optionally substituted alkenyl, optionally substituted alkynyl, optionally substituted carbocyclyl, or optionally substituted aryl; to form a compound of Formula (6):

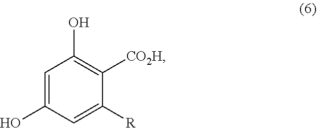

wherein R is hydrogen, optionally substituted acyl, optionally substituted alkyl, optionally substituted alkenyl, optionally substituted alkynyl, optionally substituted carbocyclyl, or optionally substituted aryl; as substrates. R is as defined in this application. In some embodiments, R is a C2-C6 optionally substituted alkyl. In some embodiments, R is a propyl or pentyl. In some embodiments, R is pentyl. In some embodiments, R is propyl. In certain embodiments, a PKC is an olivetolic acid cyclase (OAC). In certain embodiments, a PKC is a divarinic acid cyclase (DAC).

As one of ordinary skill in the art would appreciate a PKC could be obtained from any source, including naturally occurring sources and synthetic sources (e.g., a non-naturally occurring PKC). In some embodiments, a PKC is from Cannabis. Non-limiting examples of PKCs include those disclosed in U.S. Pat. Nos. 9,611,460; 10,059,971; and U.S. Patent No. 2019/0169661, which are incorporated by reference in this application in their entireties.

In some embodiments, a PKC is an OAC. As used in this application, an “OAC” refers to an enzyme that is capable of catalyzing the formation of olivetolic acid (OA). In some embodiments, an OAC is an enzyme that is capable of using a substrate of Formula (4a) (3,5,7-trioxododecanoyl-CoA):

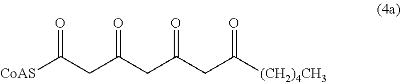

to form a compound of Formula (6a) (olivetolic acid):

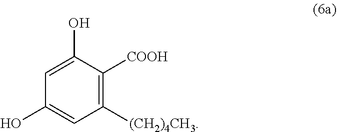

Olivetolic acid cyclase from C. sativa (CsOAC) is a 101 amino acid enzyme that performs non-decaboxylative cyclization of the tetraketide product of olivetol synthase (FIG. 4 Structure 4a) via aldol condensation to form olivetolic acid (FIG. 4 Structure 6a). CsOAC was identified and characterized by Gagne et al. (PNAS 2012) via transcriptome mining, and its cyclization function was recapitulated in vitro to demonstrate that CsOAC is required for formation of olivetolic acid in C. sativa. A crystal structure of the enzyme was published by Yang et al. (FEBS J. 2016 March; 283(6):1088-106), which revealed that the enzyme is a homodimer and belongs to the α+β barrel (DABB) superfamily of protein folds. CsOAC is the only known plant polyketide cyclase. Multiple fungal Type III polyketide synthases have been identified that perform both polyketide synthase and cyclization functions (Funa et al., J Biol Chem. 2007 May 11; 282(19):14476-81); however, in plants such a dual function enzyme has not yet been discovered.

A non-limiting example of an amino acid sequence of an OAC in C. sativa is provided by UniProtKB—I6WU39 (SEQ ID NO: 1), which catalyzes the formation of olivetolic acid (OA) from 3,5,7-Trioxododecanoyl-CoA.

The sequence of UniProtKB—I6WU39 (SEQ ID NO: 1) is:

MAVKHLIVLKFKDEITEAQKEEFFKTYVNLVNIIPAMKDVYWGKDVTQKN
KEEGYTHIVEVTFESVETIQDYIIHPAHVGFGDVYRSFWEKLLIFDYTPR
K.

A non-limiting example of a nucleic acid sequence encoding C. sativa OAC is:

(SEQ ID NO: 2)
atggcagtgaagcatttgattgtattgaagttcaaagatgaaatcacaga
agcccaaaaggaagaatttttcaagacgtatgtgaatcttgtgaatatca
tcccagccatgaaagatgtatactggggtaaagatgtgactcaaaagaat
aaggaagaagggtacactcacatagttgaggtaacatttgagagtgtgga
gactattcaggactacattattcatcctgcccatgttggatttggagatg
tctatcgttctttctgggaaaaacttctcatttttgactacacaccacga
aag.

### Prenyltransferase (PT)

A host cell described in this application may comprise a prenyltransferase (PT). As used in this application, a “PT” refers to an enzyme that is capable of transferring prenyl groups to acceptor molecule substrates. Non-limiting examples of prenyltransferases are described in PCT Publication No. WO2018200888 (e.g., CsPT4), U.S. Pat. No. 8,884,100 (e.g., CsPT1); Canadian Patent No. CA2718469; Valliere et al., Nat Commun. 2019 Feb. 4; 10(1):565; and Luo et al., Nature 2019 March; 567(7746):123-126, which are incorporated by reference in their entireties. In some embodiments, a PT is capable of producing cannabigerolic acid (CBGA), cannabigerovarinic acid (CBGVA), or other cannabinoids or cannabinoid-like substances. In some embodiments, a PT is cannabigerolic acid synthase (CBGAS). In some embodiments, a PT is cannabigerovarinic acid synthase (CBGVAS).

In some embodiments, the PT is an NphB prenyltransferase. See, e.g., U.S. Pat. No. 7,544,498; and Kumano et al., Bioorg Med Chem. 2008 Sep. 1; 16(17): 8117-8126, which are incorporated by reference in this application in their entireties. In some embodiments, a PT corresponds to NphB from Streptomyces sp. (see, e.g., UniprotKB Accession No. Q4R2T2; see also SEQ ID NO: 2 of U.S. Pat. No. 7,361,483). The protein sequence corresponding to UniprotKB Accession No. Q4R2T2 is provided by SEQ ID NO: 8:

(SEQ ID NO: 8)
MSEAADVERVYAAMEEAAGLLGVACARDKIYPLLSTFQDTLVEGGSVVVF
SMASGRHSTELDFSISVPTSHGDPYATVVEKGLFPATGHPVDDLLADTQK
HLPVSMFAIDGEVTGGFKKTYAFFPTDNMPGVAELSAIPSMPPAVAENAE
LFARYGLDKVQMTSMDYKKRQVNLYFSELSAQTLEAESVLALVRELGLHV
PNELGLKFCKRSFSVYPTLNWETGKIDRLCFAVISNDPTLVPSSDEGDIE
KFHNYATKAPYAYVGEKRTLVYGLTLSPKEEYYKLGAYYHITDVQRGLLK
AFDSLED.

A non-limiting example of a nucleic acid sequence encoding NphB is:

(SEQ ID NO: 9)
atgtcagaagccgcagatgtcgaaagagtttacgccgctatggaagaagc
cgccggtttgttaggtgttgcctgtgccagagataagatctacccattgt
tgtctacttttcaagatacattagttgaaggtggttcagttgttgttttc
tctatggcttcaggtagacattctacagaattggatttctctatctcagt
tccaacatcacatggtgatccatacgctactgttgttgaaaaaggtttat
ttccagcaacaggtcatccagttgatgatttgttggctgatactcaaaag
catttgccagtttctatgtttgcaattgatggtgaagttactggtggttt
caagaaaacttacgctttctttccaactgataacatgccaggtgttgcag
aattatctgctattccatcaatgccaccagctgttgcagaaaatgcagaa
ttatttgctagatacggtttggataaggttcaaatgacatctatggatta
caagaaaagacaagttaatttgtacttttctgaattatcagcacaaactt
tggaagctgaatcagttttggcattagttagagaattgggtttacatgtt
ccaaacgaattgggtttgaagttttgtaaaagatctttctcagtttatcc
aactttaaactgggaaacaggcaagatcgatagattatgtttcgcagtta
tctctaacgatccaacattggttccatcttcagatgaaggtgatatcgaa
aagtttcataactacgctactaaagcaccatatgcttacgttggtgaaaa
gagaacattagtttatggtttgactttatcaccaaaggaagaatactaca
agttgggtgcttactaccacattaccgacgtacaaagaggtttattgaaa
gcattcgatagtttagaagactaa.

In other embodiments, a PT corresponds to CsPT1, which is disclosed as SEQ ID NO:2 in U.S. Pat. No. 8,884,100 (C. sativa; corresponding to SEQ ID NO: 10 in this application):

(SEQ ID NO: 10)
MGLSSVCTFSFQTNYHTLLNPHNNNPKTSLLCYRHPKTPIKYSYNNFPSK
HCSTKSFHLQNKCSESLSIAKNSIRAATTNQTEPPESDNHSVATKILNFG
KACWKLQRPYTIIAFTSCACGLFGKELLHNTNLISWSLMFKAFFFLVAIL
CIASFTTTINQIYDLHIDRINKPDLPLASGEISVNTAWIMSIIVALFGLI
ITIKMKGGPLYIFGYCFGIFGGIVYSVPPFRWKQNPSTAFLLNFLAHIIT
NFTFYYASRAALGLPFELRPSFTFLLAFMKSMGSALALIKDASDVEGDTK
FGISTLASKYGSRNLTLFCSGIVLLSYVAAILAGIIWPQAFNSNVMLLSH
AILAFWLILQTRDFALTNYDPEAGRRFYEFMWKLYYAEYLVYVFI.

In some embodiments, a PT corresponds to CsPT4, which is disclosed as SEQ ID NO:1 in PCT Publication No. WO2019071000, corresponding to SEQ ID NO: 11 in this application:

(SEQ ID NO: 11)
MGLSLVCTFSFQTNYHTLLNPHNKNPKNSLLSYQHPKTPIIKSSYDNFPS
KYCLTKNFHLLGLNSHNRISSQSRSIRAGSDQIEGSPHHESDNSIATKIL
NFGHTCWKLQRPYVVKGMISIACGLFGRELFNNRHLFSWGLMWKAFFALV
PILSFNFFAAIMNQIYDVDIDRINKPDLPLVSGEMSIETAWILSIIVALT
GLIVTIKLKSAPLFVFIYIFGIFAGFAYSVPPIRWKQYPFTNFLITISSH
VGLAFTSYSATTSALGLPFVWRPAFSFIIAFMTVMGMTIAFAKDISDIEG
DAKYGVSTVATKLGARNMTFVVSGVLLLNYLVSISIGIIWPQVFKSNIMI
LSHAILAFCLIFQTRELALANYASAPSRQFFEFIWLLYYAEYFVYVFI.

In some embodiments, a PT corresponds to a truncated CsPT4, which is provided as SEQ ID NO: 12:

(SEQ ID NO: 12)
MSAGSDQIEGSPHHESDNSIATKILNFGHTCWKLQRPYVVKGMISIACGL
FGRELFNNRHLFSWGLMWKAFFALVPILSFNFFAAIMNQIYDVDIDRINK
PDLPLVSGEMSIETAWILSIIVALTGLIVTIKLKSAPLFVFIYIFGIFAG
FAYSVPPIRWKQYPFTNFLITISSHVGLAFTSYSATTSALGLPFVWRPAF
SFIIAFMTVMGMTIAFAKDISDIEGDAKYGVSTVATKLGARNMTFVVSGV
LLLNYLVSISIGIIWPQVFKSNIMILSHAILAFCLIFQTRELALANYASA
PSRQFFEFIWLLYYAEYFVYVFI.

Functional expression of paralog C. sativa CBGAS enzymes in S. cerevisiae and production of the major cannabinoid CBGA has been reported (U.S. Patent Publication 2012/0144523, and Luo et al. Nature, 2019 March; 567(7746):123-126). Luo et al. reported the production of CBGA in S. cerevisiae by expressing a truncated version of a C. sativa CBGAS, CsPT4, with its native signal peptide removed. Without being bound by a particular theory, the integral-membrane nature of C. sativa CBGAS enzymes may render functional expression of C. sativa CBGAS enzymes in heterologous hosts challenging. Removal of transmembrane domain(s) or signal sequences or use of prenyltransferases that are not associated with the membrane and are not integral membrane proteins may facilitate increased interaction between the enzyme and available substrate, for example in the cellular cytosol and/or in organelles that may be targeted using peptides that confer localization.

In some embodiments, the PT is a soluble PT. In some embodiments, the PT is a cytosolic PT. In some embodiments, the PT is a secreted protein. In some embodiments, the PT is not a membrane-associated protein. In some embodiments, the PT is not an integral membrane protein. In some embodiments, the PT does not comprise a transmembrane domain or a predicted transmembrane. In some embodiments, the PT may be primarily detected in the cytosol (e.g., detected in the cytosol to a greater extent than detected associated with the cell membrane). In some embodiments, the PT is a protein from which one or more transmembrane domains have been removed and/or mutated (e.g., by truncation, deletions, substitutions, insertions, and/or additions) so that the PT localizes or is predicted to localize in the cytosol of the host cell, or to cytosolic organelles within the host cell, or, in the case of bacterial hosts, in the periplasm. In some embodiments, the PT is a protein from which one or more transmembrane domains have been removed or mutated (e.g., by truncation, deletions, substitutions, insertions, and/or additions) so that the PT has increased localization to the cytosol, organelles, or periplasm of the host cell, as compared to membrane localization.

Within the scope of the term “transmembrane domains” are predicted or putative transmembrane domains in addition to transmembrane domains that have been empirically determined. In general, transmembrane domains are characterized by a region of hydrophobicity that facilitates integration into the cell membrane. Methods of predicting whether a protein is a membrane protein or a membrane-associated protein are known in the art and may include, for example amino acid sequence analysis, hydropathy plots, and/or protein localization assays.

In some embodiments, the PT is a protein from which a signal sequence has been removed and/or mutated so that the PT is not directed to the cellular secretory pathway. In some embodiments, the PT is a protein from which a signal sequence has been removed and/or mutated so that the PT is localized to the cytosol or has increased localization to the cytosol (e.g., as compared to the secretory pathway).

In some embodiments, the PT is a secreted protein. In some embodiments, the PT contains a signal sequence.

In some embodiments, a PT is a fusion protein. For example, a PT may be fused to one or more genes in the metabolic pathway of a host cell. In certain embodiments, a PT may be fused to mutant forms of one or more genes in the metabolic pathway of a host cell.

In some embodiments, a PT described in this application transfers one or more prenyl groups to any of positions 1, 2, 3, 4, or 5 in a compound of Formula (6), shown below:

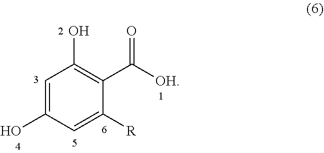

In some embodiments, the PT transfers a prenyl group to any of positions 1, 2, 3, 4, or 5 in a compound of Formula (6), shown below:

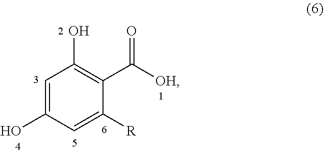

to form a compound of one or more of Formula (8w), Formula (8x), Formula (8′), Formula (8y), Formula (8z):

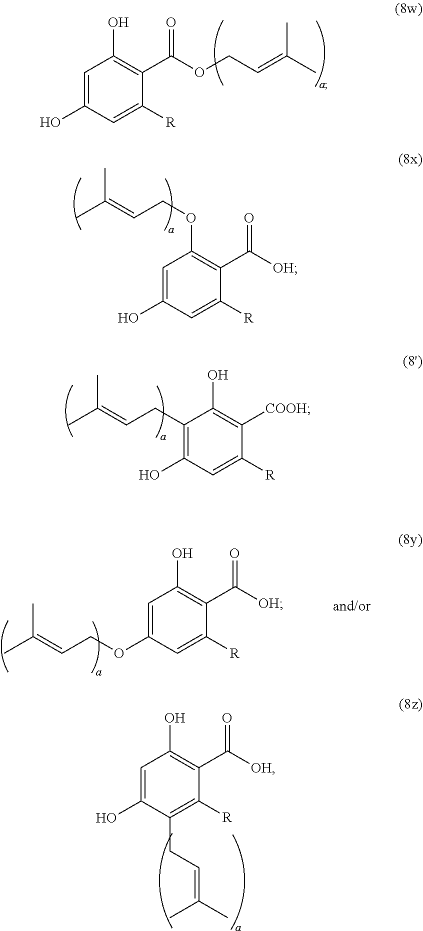

or a pharmaceutically acceptable salt, solvate, hydrate, polymorph, co-crystal, tautomer, stereoisomer, isotopically labeled derivative, or prodrug thereof, wherein a is 1, 2, 3, 4, 5, 6, 7, 8, 9, or 10.

### Variants

Aspects of the disclosure relate to nucleic acids encoding any of the polypeptides (e.g., AAE, PKS, PKC, PT, or TS) described in this application. In some embodiments, a nucleic acid encompassed by the disclosure is a nucleic acid that hybridizes under high or medium stringency conditions to a nucleic acid encoding an AAE, PKS, PKC, PT, or TS and is biologically active. For example, high stringency conditions of 0.2 to 1×SSC at 65° C. followed by a wash at 0.2×SSC at 65° C. can be used. In some embodiments, a nucleic acid encompassed by the disclosure is a nucleic acid that hybridizes under low stringency conditions to a nucleic acid encoding an AAE, PKS, PKC, PT, or TS and is biologically active. For example, low stringency conditions of 6×SSC at room temperature followed by a wash at 2×SSC at room temperature can be used. Other hybridization conditions include 3×SSC at 40 or 50° C., followed by a wash in 1 or 2×SSC at 20, 30, 40, 50, 60, or 65° C.

Hybridizations can be conducted in the presence of formaldehyde, e.g., 10%, 20%, 30% 40% or 50%, which further increases the stringency of hybridization. Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays,” Elsevier, New York provide a basic guide to nucleic acid hybridization.

Variants of enzyme sequences described in this application (e.g., AAE, PKS, PKC, PT, or TS, including nucleic acid or amino acid sequences) are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” which is used interchangeably in this disclosure with the term “percent identity,” as known in the art, refers to a relationship between the sequences of two polypeptides or polynucleotides, as determined by sequence comparison (alignment). In some embodiments, sequence identity is determined across the entire length of a sequence (e.g., AAE, PKS, PKC, PT, or TS sequence). In some embodiments, sequence identity is determined over a region (e.g., a stretch of amino acids or nucleic acids, e.g., the sequence spanning an active site) of a sequence (e.g., AAE, PKS, PKC, PT, or TS sequence). For example, in some embodiments, sequence identity is determined over a region corresponding to at least 30%, at least 40%, at least 50%, at least 60%, at least 70%, at least 80%, at least 90%, at least 95%, or over 100% of the length of the reference sequence.

Identity measures the percent of identical matches between the smaller of two or more sequences with gap alignments (if any) addressed by a particular mathematical model, algorithm, or computer program.

Identity of related polypeptides or nucleic acid sequences can be readily calculated by any of the methods known to one of ordinary skill in the art. The percent identity of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described in this application. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453) using default parameters.

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539) using default parameters.

As used in this application, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using amino acid sequence alignment tools known in the art.

As used in this application, variant sequences may be homologous sequences. As used in this application, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between). Homologous sequences include but are not limited to paralogous or orthologous sequences. Paralogous sequences arise from duplication of a gene within a genome of a species, while orthologous sequences diverge after a speciation event.

In some embodiments, a polypeptide variant (e.g., AAE, PKS, PKC, PT, or TS enzyme variant) comprises a domain that shares a secondary structure (e.g., alpha helix, beta sheet) with a reference polypeptide (e.g., a reference AAE, PKS, PKC, PT, or TS enzyme). In some embodiments, a polypeptide variant (e.g., AAE, PKS, PKC, PT, or TS enzyme variant) shares a tertiary structure with a reference polypeptide (e.g., a reference AAE, PKS, PKC, PT, or TS enzyme). As a non-limiting example, a polypeptide variant (e.g., AAE, PKS, PKC, PT, or TS enzyme) may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets), or have the same tertiary structure as a reference polypeptide. For example, a loop may be located between a beta sheet and an alpha helix, between two alpha helices, or between two beta sheets. Homology modeling may be used to compare two or more tertiary structures.

Functional variants of the recombinant AAE, PKS, PKC, PT, or TS enzyme disclosed in this application are encompassed by the present disclosure. For example, functional variants may bind one or more of the same substrates or produce one or more of the same products. Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain.

Homology modeling may also be used to identify amino acid residues that are amenable to mutation (e.g., substitution, deletion, and/or insertion) without affecting function. A non-limiting example of such a method may include use of position-specific scoring matrix (PSSM) and an energy minimization protocol.

Position-specific scoring matrix (PSSM) uses a position weight matrix to identify consensus sequences (e.g., motifs). PSSM can be conducted on nucleic acid or amino acid sequences. Sequences are aligned and the method takes into account the observed frequency of a particular residue (e.g., an amino acid or a nucleotide) at a particular position and the number of sequences analyzed. See, e.g., Stormo et al., Nucleic Acids Res. 1982 May 11; 10(9):2997-3011. The likelihood of observing a particular residue at a given position can be calculated. Without being bound by a particular theory, positions in sequences with high variability may be amenable to mutation (e.g., substitution, deletion, and/or insertion; e.g., PSSM score ≥0) to produce functional homologs.

PSSM may be paired with calculation of a Rosetta energy function, which determines the difference between the wild-type and the single-point mutant. The Rosetta energy function calculates this difference as (ΔΔG_{calc}). With the Rosetta function, the bonding interactions between a mutated residue and the surrounding atoms are used to determine whether a mutation increases or decreases protein stability. For example, a mutation that is designated as favorable by the PSSM score (e.g. PSSM score ≥0), can then be analyzed using the Rosetta energy function to determine the potential impact of the mutation on protein stability. Without being bound by a particular theory, potentially stabilizing amino acid mutations are desirable for protein engineering (e.g., production of functional homologs). In some embodiments, a potentially stabilizing amino acid mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63(2):337-346. Doi: 10.1016/j.molcel.2016.06.012.

In some embodiments, a coding sequence comprises an amino acid mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions relative to a reference coding sequence. In some embodiments, the coding sequence comprises an amino acid mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference coding sequence. As will be understood by one of ordinary skill in the art, a substitution, insertion, or deletion within a codon may or may not change the amino acid that is encoded by the codon due to degeneracy of the genetic code. In some embodiments, the one or more substitutions, insertions, or deletions in the coding sequence do not alter the amino acid sequence of the coding sequence relative to the amino acid sequence of a reference polypeptide.

In some embodiments, the one or more mutations in a sequence do alter the amino acid sequence of the corresponding polypeptide relative to the amino acid sequence of a reference polypeptide. In some embodiments, the one or more mutations alters the amino acid sequence of the polypeptide relative to the amino acid sequence of a reference polypeptide and alter (enhance or reduce) an activity of the polypeptide relative to the reference polypeptide.

The activity (e.g., specific activity) of any of the recombinant polypeptides described in this application (e.g., AAE, PKS, PKC, PT, or TS) may be measured using routine methods. As a non-limiting example, a recombinant polypeptide's activity may be determined by measuring its substrate specificity, product(s) produced, the concentration of product(s) produced, or any combination thereof. As used in this application, “specific activity” of a recombinant polypeptide refers to the amount (e.g., concentration) of a particular product produced for a given amount (e.g., concentration) of the recombinant polypeptide per unit time.

The skilled artisan will also realize that mutations in a coding sequence may result in conservative amino acid substitutions to provide functionally equivalent variants of the foregoing polypeptides, e.g., variants that retain the activities of the polypeptides. As used in this application, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 4). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. As used in this application “conservative substitution” is used interchangeably with “conservative amino acid substitution” and refers to any one of the amino acid substitutions provided in Table 4.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 4
Conservative Amino Acid Substitutions

| Original | | Conservative Amino |
|---|---|---|
| Residue | R Group Type | Acid Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a recombinant polypeptide (e.g., AAE, PKS, PKC, PT, or TS) variant having a desired property and/or activity can be made by alteration of the coding sequence of the polypeptide (e.g., AAE, PKS, PKC, PT, or TS). Similarly, conservative amino acid substitutions in the amino acid sequence of a polypeptide to produce functionally equivalent variants of the polypeptide typically are made by alteration of the coding sequence of the recombinant polypeptide (e.g., AAE, PKS, PKC, PT, or TS).

Mutations (e.g., substitutions, insertions, additions, or deletions) can be made in a nucleic acid sequence by a variety of methods known to one of ordinary skill in the art. For example, mutations (e.g., substitutions, insertions, additions, or deletions) can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by CRISPR, or by insertions, such as insertion of a tag (e.g., a HIS tag or a GFP tag). Mutations can include, for example, substitutions, insertions, additions, deletions, and translocations, generated by any method known in the art. Methods for producing mutations may be found in in references such as Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 January; 29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a protein that has undergone circular permutation, the linear amino acid sequence of the protein would differ from a reference protein that has not undergone circular permutation. However, one of ordinary skill in the art would be able to determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

### Expression of Nucleic Acids in Host Cells

Aspects of the present disclosure relate to recombinant enzymes, functional modifications and variants thereof, as well as their uses. For example, the methods described in this application may be used to produce cannabinoids and/or cannabinoid precursors. The methods may comprise using a host cell comprising an enzyme disclosed in this application, cell lysate, isolated enzymes, or any combination thereof. Methods comprising recombinant expression of genes encoding an enzyme disclosed in this application in a host cell are encompassed by the present disclosure. In vitro methods comprising reacting one or more cannabinoid precursors or cannabinoids in a reaction mixture with an enzyme disclosed in this application are also encompassed by the present disclosure. In some embodiments, the enzyme is a TS.

A nucleic acid encoding any of the recombinant polypeptides (e.g., AAE, PKS, PKC, PT, or TS enzyme) described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

A vector encoding any of the recombinant polypeptides (e.g., AAE, PKS, PKC, PT, or TS enzyme) described in this application may be introduced into a suitable host cell using any method known in the art. Non-limiting examples of yeast transformation protocols are described in Gietz et al., Yeast transformation can be conducted by the LiAc/SS Carrier DNA/PEG method. Methods Mol Biol. 2006; 313:107-20, which is hereby incorporated by reference in its entirety. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

In some embodiments, a vector replicates autonomously in the cell. In some embodiments, a vector integrates into a chromosome within a cell. A vector can contain one or more endonuclease restriction sites that are cut by a restriction endonuclease to insert and ligate a nucleic acid containing a gene described in this application to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell (e.g., microbe), such as a yeast cell. In some embodiments, the nucleic acid sequence of a gene described in this application is inserted into a cloning vector so that it is operably joined to regulatory sequences and, in some embodiments, expressed as an RNA transcript. In some embodiments, the vector contains one or more markers, such as a selectable marker as described in this application, to identify cells transformed or transfected with the recombinant vector. In some embodiments, a host cell has already been transformed with one or more vectors. In some embodiments, a host cell that has been transformed with one or more vectors is subsequently transformed with one or more vectors. In some embodiments, a host cell is transformed simultaneously with more than one vector. In some embodiments, a cell that has been transformed with a vector or an expression cassette incorporates all or part of the vector or expression cassette into its genome. In some embodiments, the nucleic acid sequence of a gene described in this application is recoded. Recoding may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not recoded.

In some embodiments, the nucleic acid encoding any of the proteins described in this application is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1, GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. This may be used, for example, to controllably induce the expression of an enzyme. In some embodiments, an inducible promoter linked to an enzyme may be used to regulate expression of the enzyme(s), for example to reduce cannabinoid production in certain scenarios (e.g., during transport of the genetically modified organism to satisfy regulatory restrictions in certain jurisdictions, or between jurisdictions, where cannabinoids may not be shipped). In some embodiments, an inducible promoter linked to an enzyme may be used to regulate expression of the enzyme(s), for example to reduce cannabinoid production in certain scenarios (e.g., during transport of the genetically modified organism to satisfy regulatory restrictions in certain jurisdictions, or between jurisdictions, where cannabinoids may not be shipped). Non-limiting examples of inducible promoters include chemically regulated promoters and physically regulated promoters. For chemically regulated promoters, the transcriptional activity can be regulated by one or more compounds, such as alcohol, tetracycline, galactose, a steroid, a metal, an amino acid, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1, ADH1, ADH2, ENO2, and SOD1.

Other inducible promoters or constitutive promoters, including synthetic promoters, that may be known to one of ordinary skill in the art are also contemplated.

The precise nature of the regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. The vectors disclosed may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this application in a heterologous organism is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

### Host Cells

The disclosed cannabinoid biosynthetic methods and host cells are exemplified with S. cerevisiae, but are also applicable to other host cells, as would be understood by one of ordinary skill in the art.

Suitable host cells include, but are not limited to: yeast cells, bacterial cells, algal cells, plant cells, fungal cells, insect cells, and animal cells, including mammalian cells. In one illustrative embodiment, suitable host cells include E. coli (e.g., Shuffle™ competent E. coli available from New England BioLabs in Ipswich, Mass.).

Other suitable host cells of the present disclosure include microorganisms of the genus Corynebacterium. In some embodiments, preferred Corynebacterium strains/species include: C. efficiens, with the deposited type strain being DSM44549, C. glutamicum, with the deposited type strain being ATCC13032, and C. ammoniagenes, with the deposited type strain being ATCC6871. In some embodiments the preferred host cell of the present disclosure is C. glutamicum.

Suitable host cells of the genus Corynebacterium, in particular of the species Corynebacterium glutamicum, are in particular the known wild-type strains: Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Komagataella phaffii, formerly known as Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as, Chlamydomonas (e.g., C. reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host strain is an industrial strain. Numerous bacterial industrial strains are known and suitable for the methods and compositions described in this application.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

The present disclosure is also suitable for use with a variety of animal cell types, including mammalian cells, for example, human (including 293, HeLa, W138, PER.C6 and Bowes melanoma cells), mouse (including 3T3, NS0, NS1, Sp2/0), hamster (CHO, BHK), monkey (COS, FRhL, Vero), insect cells, for example fall armyworm (including Sf9 and Sf21), silkmoth (including BmN), cabbage looper (including BTI-Tn-5B1-4) and common fruit fly (including Schneider 2), and hybridoma cell lines.

In various embodiments, strains that may be used in the practice of the disclosure including both prokaryotic and eukaryotic strains, and are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). The present disclosure is also suitable for use with a variety of plant cell types. In some embodiments, the plant is of the Cannabis genus in the family Cannabaceae. In certain embodiments, the plant is of the species Cannabis sativa, Cannabis indica, or Cannabis ruderalis. In other embodiments, the plant is of the genus Nicotiana in the family Solanaceae. In certain embodiments, the plant is of the species Nicotiana rustica.

The term “cell,” as used in this application, may refer to a single cell or a population of cells, such as a population of cells belonging to the same cell line or strain. Use of the singular term “cell” should not be construed to refer explicitly to a single cell rather than a population of cells. The host cell may comprise genetic modifications relative to a wild-type counterpart. Reduction of gene expression and/or gene inactivation in a host cell may be achieved through any suitable method, including but not limited to, deletion of the gene, introduction of a point mutation into the gene, selective editing of the gene and/or truncation of the gene. For example, polymerase chain reaction (PCR)-based methods may be used (see, e.g., Gardner et al., Methods Mol Biol. 2014; 1205:45-78). As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104). A gene may also be edited through of the use of gene editing technologies known in the art, such as CRISPR-based technologies.

### Culturing of Host Cells

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cell. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place that involves a living organism or part of a living organism. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermenters, bioreactors agitated by rotating mixing devices, chemostats, bioreactors agitated by shaking devices, airlift fermenters, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermenters, and coated beads (e.g., beads coated with serum proteins, nitrocellulose, or carboxymethyl cellulose to prevent cell attachment).

In some embodiments, the bioreactor includes a cell culture system where the cell (e.g., yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermenter includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., cannabinoid or cannabinoid precursor) may display some differences from the substrate in terms of solubility, toxicity, cellular accumulation and secretion and in some embodiments can have different fermentation kinetics.

In some embodiments, the cells of the present disclosure are adapted to produce cannabinoids or cannabinoid precursors in vivo. In some embodiments, the cells are adapted to secrete one or more enzymes for cannabinoid synthesis (e.g., AAE, PKS, PKC, PT, or TS). In some embodiments, the cells of the present disclosure are lysed, and the remaining lysates are recovered for subsequent use. In such embodiments, the secreted or lysed enzyme can catalyze reactions for the production of a cannabinoid or precursor by bioconversion in an in vitro or ex vivo process. In some embodiments, any and all conversions described in this application can be conducted chemically or enzymatically, in vitro or in vivo.

In some embodiments, the host cells of the present disclosure are adapted to produce cannabinoids or cannabinoid precursors in vivo. In some embodiments, the host cells are adapted to secrete one or more cannabinoid pathway substrates, intermediates, and/or terminal products (e.g., olivetol, THCA, THC, CBDA, CBD, CBGA, CBGVA, THCVA, CBDVA, CBCVA, or CBCA). In some embodiments, the host cells of the present disclosure are lysed, and the lysate is recovered for subsequent use. In such embodiments, the secreted substrates, intermediates, and/or terminal products may be recovered from the culture media.

### Purification and Further Processing

In some embodiments, any of the methods described in this application may include isolation and/or purification of the cannabinoids and/or cannabinoid precursors produced (e.g., produced in a bioreactor). For example, the isolation and/or purification can involve one or more of cell lysis, centrifugation, extraction, column chromatography, distillation, crystallization, and lyophilization.

The methods described in this application encompass production of any cannabinoid or cannabinoid precursor known in the art. Cannabinoids or cannabinoid precursors produced by any of the recombinant cells disclosed in this application or any of the in vitro methods described in this application may be identified and extracted using any method known in the art. Mass spectrometry (e.g., LC-MS, GC-MS) is a non-limiting example of a method for identification and may be used to extract a compound of interest.

In some embodiments, any of the methods described in this application further comprise decarboxylation of a cannabinoid or cannabinoid precursor. As a non-limiting example, the acid form of a cannabinoid or cannabinoid precursor may be heated (e.g., at least 90° C.) to decarboxylate the cannabinoid or cannabinoid precursor. See, e.g., U.S. Pat. Nos. 10,159,908, 10,143,706, 9,908,832 and 7,344,736. See also, e.g., Wang et al., Cannabis Cannabinoid Res. 2016; 1(1): 262-271.

### Compositions, Kits, and Administration

The present disclosure provides compositions, including pharmaceutical compositions, comprising a cannabinoid or a cannabinoid precursor, or pharmaceutically acceptable salt thereof, produced by any of the methods described in this application, and optionally a pharmaceutically acceptable excipient.

In certain embodiments, a cannabinoid or cannabinoid precursor described in this application is provided in an effective amount in a composition, such as a pharmaceutical composition. In certain embodiments, the effective amount is a therapeutically effective amount. In certain embodiments, the effective amount is a prophylactically effective amount.

Compositions, such as pharmaceutical compositions, described in this application can be prepared by any method known in the art. In general, such preparatory methods include bringing a compound described in this application (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described in this application will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition. Exemplary excipients include diluents, dispersing and/or granulating agents, surface active agents and/or emulsifiers, disintegrating agents, binding agents, preservatives, buffering agents, lubricating agents, and/or oils (e.g., synthetic oils, semi-synthetic oils) as disclosed in this application.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic or semi-synthetic oils include, but are not limited to, butyl stearate, medium chain triglycerides (such as caprylic triglyceride and capric triglyceride), cyclomethicone, diethyl sebacate, dimethicone 360, isopropyl myristate, mineral oil, octyldodecanol, oleyl alcohol, silicone oil, and mixtures thereof. In certain embodiments, exemplary synthetic oils comprise medium chain triglycerides (such as caprylic triglyceride and capric triglyceride).

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described in this application are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described in this application with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described in this application may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described in this application include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described in this application.

A pharmaceutical composition described in this application can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Although the descriptions of pharmaceutical compositions provided in this application are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided in this application are typically formulated in dosage unit form for ease of administration and uniformity of dosage. It will be understood, however, that the total daily usage of the compositions described in this application will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided in this application can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

In some embodiments, compounds or compositions disclosed in this application are formulated and/or administered in nanoparticles. Nanoparticles are particles in the nanoscale. In some embodiments, nanoparticles are less than 1 μm in diameter. In some embodiments, nanoparticles are between about 1 and 100 nm in diameter. Nanoparticles include organic nanoparticles, such as dendrimers, liposomes, or polymeric nanoparticles. Nanoparticles also include inorganic nanoparticles, such as fullerenes, quantum dots, and gold nanoparticles. Compositions may comprise an aggregate of nanoparticles. In some embodiments, the aggregate of nanoparticles is homogeneous, while in other embodiments the aggregate of nanoparticles is heterogeneous.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described in this application. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described in this application includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described in this application. In certain embodiments, a dose described in this application includes independently between 1 mg and 3 mg, inclusive, of a compound described in this application. In certain embodiments, a dose described in this application includes independently between 3 mg and 10 mg, inclusive, of a compound described in this application. In certain embodiments, a dose described in this application includes independently between 10 mg and 30 mg, inclusive, of a compound described in this application. In certain embodiments, a dose described in this application includes independently between 30 mg and 100 mg, inclusive, of a compound described in this application.

Dose ranges as described in this application provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described in this application, can be administered in combination with one or more additional pharmaceutical agents (e.g., therapeutically and/or prophylactically active agents). The compounds or compositions can be administered in combination with additional pharmaceutical agents that improve their activity, improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described in this application including a compound described in this application and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described in this application in a single dose or administered separately in different doses. The particular combination to employ in a regimen will take into account compatibility of the compound described in this application with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In some embodiments, one or more of the compositions described in this application are administered to a subject. In certain embodiments, the subject is an animal. The animal may be of either sex and may be at any stage of development. In certain embodiments, the subject is a human. In other embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate.

Also encompassed by the disclosure are kits (e.g., pharmaceutical packs). The kits provided may comprise a composition, such as a pharmaceutical composition, or a compound described in this application and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described in this application. In some embodiments, the pharmaceutical composition or compound described in this application provided in the first container and the second container a combined to form one unit dosage form.

Thus, in one aspect, provided are kits including a first container comprising a compound or composition described in this application. In certain embodiments, the kits are useful for treating a disease in a subject in need thereof. In certain embodiments, the kits are useful for preventing a disease in a subject in need thereof. In certain embodiments, the kits are useful for reducing the risk of developing a disease in a subject in need thereof.

In certain embodiments, a kit described in this application further includes instructions for using the kit. A kit described in this application may also include information as required by a regulatory agency such as the U.S. Food and Drug Administration (FDA). In certain embodiments, the information included in the kits is prescribing information. In certain embodiments, the kits and instructions provide for treating a disease in a subject in need thereof. In certain embodiments, the kits and instructions provide for preventing a disease in a subject in need thereof. In certain embodiments, the kits and instructions provide for reducing the risk of developing a disease in a subject in need thereof. A kit described in this application may include one or more additional pharmaceutical agents described in this application as a separate composition.

In some embodiments, the compositions include consumer product, such as comestible, cosmetic, toiletry, potable, inhalable, and wellness products. Exemplary consumer products include salves, waxes, powdered concentrates, pastes, extracts, tinctures, powders, oils, capsules, skin patches, sublingual oral dose drops, mucous membrane oral spray doses, makeup, perfume, shampoos, cosmetic soaps, cosmetic creams, skin lotions, aromatic essential oils, massage oils, shaving preparations, oils for toiletry purposes, lip balm, cosmetic oils, facial washes, moisturizing creams, moisturizing body lotions, moisturizing face lotions, bath salts, bath gels, bath soaps in liquid form, shower gels, bath bombs, hair care preparations, shampoos, conditioner, chocolate bars, brownies, chocolates, cookies, crackers, cakes, cupcakes, puddings, honey, chocolate confections, frozen confections, fruit-based confectionery, sugar confectionery, gummy candies, dragées, pastries, cereal bars, chocolate, cereal based energy bars, candy, ice cream, tea-based beverages, coffee-based beverages, and herbal infusions.

The present invention is further illustrated by the following Examples, which in no way should be construed as limiting. The entire contents of all of the references (including literature references, issued patents, published patent applications, and co-pending patent applications) cited throughout this application are hereby expressly incorporated by reference. If a reference incorporated in this application contains a term whose definition is incongruous or incompatible with the definition of same term as defined in the present disclosure, the meaning ascribed to the term in this disclosure shall govern. However, mention of any reference, article, publication, patent, patent publication, and patent application cited in this application is not, and should not be taken as an acknowledgment or any form of suggestion that they constitute valid prior art or form part of the common general knowledge in any country in the world.

### Examples

### Example 1: Primary High-Throughput Screen to Identify Functional Expression of

Cannabichromenic Acid Synthases (CBCASs)

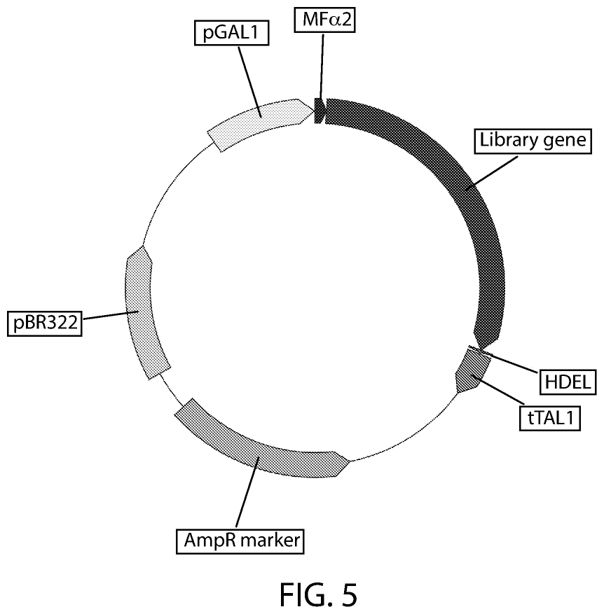

To identify CBCAS genes that can be functionally expressed in host cells, a library of approximately 3000 candidate CBCAS genes was designed based on internal codebases and domain knowledge, sampled across enzyme families, ecological niches, and structural homologies. Protein sequences were recoded in silico for expression in S. cerevisiae and synthesized in the integrative yeast expression vector shown in FIG. 5. Each candidate enzyme expression construct was transformed into an S. cerevisiae CEN.PK strain that also expressed a prenyltransferase enzyme capable of catalyzing reaction R⁴ in FIG. 2. Strain t616313, expressing GFP, was included in the library screen as a negative control for enzyme activity.

A putative C. sativa CBCAS enzyme that was previously disclosed was not found to be active. Instead, a C. sativa THCAS enzyme (set forth in SEQ ID NO:23) was found to demonstrate CBCAS activity in addition to THCAS activity using the assays described in this Example, and was accordingly used as a positive control for CBCAS activity (strain t616315). All candidate enzymes in the library, as well as the enzyme expressed by positive control strain t616315, included an N-terminal MFalpha2 signal peptide (SEQ ID NO: 16), (with a methionine residue added at the N-terminus of the MFalpha2 signal peptide), and a C-terminal HDEL signal peptide (SEQ ID NO: 17).

An assay to detect TS activity was conducted as follows: each thawed glycerol stock of candidate CBCAS transformants was stamped into a well of YEP+4% dextrose media. Samples were incubated at 30° C. in a shaking incubator for 2 days. A portion of each of the resulting cultures was stamped into a well of YEP+4% galactose+1 mM olivetolic acid (FIG. 1 Structure 6a). Samples were incubated at 20° C. and shaken in a shaking incubator for 4 days. Every 24 hours during those 4 days, 2% galactose and 1 mM olivetolic acid were spiked into the cultures. Sodium citrate buffer adjusted to pH 5.5 was added to each well at a final concentration of 100 mM. Samples were incubated at 20° C. and shaken in a shaking incubator for 2 days. A portion of each of the resulting production cultures was stamped into a well of phosphate buffered saline (PBS). Optical measurements were taken on a plate reader, with absorbance measured at 600 nm and fluorescence at 528 nm with 485 nm excitation. Samples were incubated at 30° C. in a shaking incubator for 2 days. 100% methanol was stamped into the production cultures in half-height deepwell plates. Plates were heat sealed and frozen. Samples were then thawed for 30 min and spun down at 4° C. A portion of the supernatant was stamped into half-area 96 well plates. CBCA, THCA, and CBDA production in the samples was quantified via liquid chromatography-mass spectrometry (LC-MS).

The library of candidate CBCAS enzymes was assayed for activity in a primary high-throughput screen using the assay described above. LC-MS analysis revealed a single “hit” CBCAS (strain t619896, expressing an A. niger protein of SEQ ID NO: 25 linked to an N-terminal MFalpha2 signal peptide (with a methionine residue added at the N-terminus of the MFalpha2 signal peptide) and a C-terminal HDEL signal peptide), that produced measurable amounts of CBCA.

Surprisingly, the candidate A. niger CBCAS enzyme has very low sequence identity with C. sativa CBCAS and THCAS enzymes. An alignment of the A. niger CBCAS enzyme (SEQ ID NO: 27 (UniProt accession No. A0A254UC34), which corresponds to SEQ ID NO: 25 plus a methionine residue at the N-terminus) with a putative C. sativa CBCAS enzyme (SEQ ID NO: 15), and a C. sativa THCAS enzyme (SEQ ID NO: 20, corresponding to UniProt accession No. I1V0C5) using BLASTP with default parameters, reveals 21.15% identity, and 21.71% identity, respectively.

To confirm the activity of the candidate CBCAS enzyme identified in the primary screen, a secondary screen was performed to verify CBCA production. The experimental protocol for the secondary screen was identical to the primary screen, except that additional biological replicates were included per strain, and replicate production cultures for each strain were separately fed 1 mM olivetolic acid or 1 mM divaric acid. All strains were screened in quadruplicate.

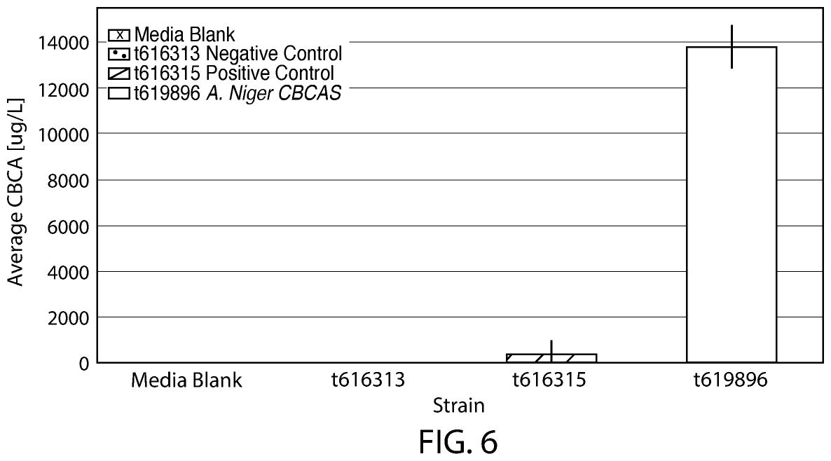

Consistent with the primary screen, the secondary screen revealed CBCAS activity for strain t619896, as shown by titers of CBCA produced by this strain (Table 5 and FIG. 6).

TABLE 5
CBCA titers from secondary screening of CBCAS
candidate enzymes in S. cerevisiae

| | | Average | Standard |
|---|---|---|---|
| | | CBCA | Deviation CBCA |
| Strain | Strain type | [μg/L] | [μg/L] |
| t616313 | Negative Control (GFP) | 0.0 | 0.0 |
| t616315 | Positive Control | 362.9 | 575.6 |
| | (C. sativa THCAS) | | |
| t619896 | Library | 13772.4 | 978.5 |
| | (A. niger CBCAS) | | |

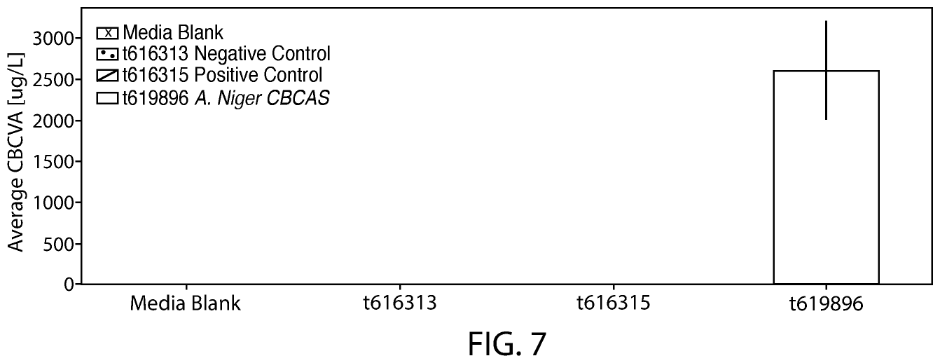

Surprisingly, strain t619896 also revealed CBCVAS activity, as shown by titers of CBCVA produced by this strain (Table 6 and FIG. 7). Strain t616315, which was used as a positive control for production of CBCA in the secondary screen, did not demonstrate CBCVAS activity (Table 6 and FIG. 7).

TABLE 6
CBCVA titers from secondary screening of CBCAS
candidate enzymes in S. cerevisiae

| | | Average | Standard |
|---|---|---|---|
| | | CBCVA | Deviation CBCVA |
| Strain | Strain type | [μg/L] | [μg/L] |
| t616313 | Negative Control (GFP) | 0 | 0 |
| t616315 | Positive Control | 0 | 0 |
| | (C. sativa THCAS) | | |
| t619896 | Library | 2609.3 | 602.5 |
| | (A. niger CBCAS) | | |

Strain t619896 also demonstrated production of THCA and CBDA, producing a terminal cannabinoid product profile consisting of 89.60% CBCA, 5.67% CBDA, and 4.73% THCA (Table 7).

TABLE 7
CBCA, THCA, and CBDA titers from secondary screening of CBCAS candidate enzymes in S. cerevisiae

| | | | Standard | | Standard | | Standard | | | |
|---|---|---|---|---|---|---|---|---|---|---|
| | | Average | Deviation | Average | Deviation | Average | Deviation | | | |
| Strain | Strain | CBCA | CBCA | THCA | THCA | CBDA | CBDA | % | % | % |
| ID | Type | [μg/L] | [μg/L] | [μg/L] | [μg/L] | [μg/L] | [μg/L] | CBCA | THCA | CBDA |
| t616313 | Negative | 0.00 | 0.00 | 506.91 | 1467.67 | 6.89 | 20.62 | 0.00 | 98.66 | 1.34 |
| | Control | | | | | | | | | |
| | (GFP) | | | | | | | | | |
| t616314 | Positive | 47.51 | 68.16 | 433.82 | 1844.40 | 719.89 | 371.17 | 3.95 | 36.12 | 59.93 |
| | Control | | | | | | | | | |
| | (C. sativa | | | | | | | | | |
| | CBDAS) | | | | | | | | | |
| t616315 | Positive | 362.95 | 575.63 | 19030.65 | 13680.86 | 142.10 | 169.23 | 1.86 | 97.41 | 0.73 |
| | Control | | | | | | | | | |
| | (C. sativa | | | | | | | | | |
| | THCAS) | | | | | | | | | |
| t619896 | Library | 13772.43 | 978.55 | 727.30 | 71.49 | 872.03 | 158.52 | 89.60 | 4.73 | 5.67 |
| | (A. niger | | | | | | | | | |
| | CBCAS) | | | | | | | | | |

Thus, out of approximately 3000 candidate genes, one CBCAS was surprisingly identified as being able to produce measurable amounts of CBCA and CBCVA when expressed in S. cerevisiae host cells. The CBCAS identified in these screens may be useful in cannabinoid biosynthesis.

### Example 2: Protein Engineering of A. niger CBCAS

To determine whether engineering of the A. niger CBCAS identified in Example 1 (corresponding to SEQ ID NO: 29 (with signal peptides); SEQ ID NO: 27 (without signal peptides and including an N-terminal methionine (UniProt accession No. A0A254UC34)); or SEQ ID NO: 25 (without signal peptides and without the N-terminal methionine)), could alter CBCAS substrate specificity, product specificity and/or amounts of products produced, point mutations were generated in A. niger CBCAS and the mutant versions of the protein were expressed in S. cerevisiae. A library containing 1047 A. niger CBCAS mutants was generated and screened. As in Example 1, each CBCAS mutant in the library, as well as the enzymes expressed by positive control strains, included an N-terminal MFalpha2 signal peptide (SEQ ID NO: 16) (with a methionine residue added at the N-terminus of the MFalpha2 signal peptide) and a C-terminal HDEL signal peptide (SEQ ID NO: 17).

Production of compounds of Formulae (9), (10), and/or (11), including compounds of Formulae (9a), (10a), and/or (11a) by strains expressing the mutated versions of A. niger CBCAS was quantified and compared to the production of the same compounds by a strain expressing wild-type A. niger CBCAS, a strain expressing a C. sativa THCAS, and a strain expressing a C. sativa CBDAS. The strains were screened using the same assay described in Example 1. Production of CBCA, THCA, and/or CBDA in the samples was quantified via LC-MS.

Of the original 1047 library members, 55 strains were elevated to a secondary screen to verify CBCA production. The experimental protocol for the secondary screen was identical to the primary screen, except that additional biological replicates were included per strain, and replicate production cultures for each strain were separately fed 1 mM boluses of olivetolic acid or 1 mM boluses of divaric acid. All strains were screened in quadruplicate.

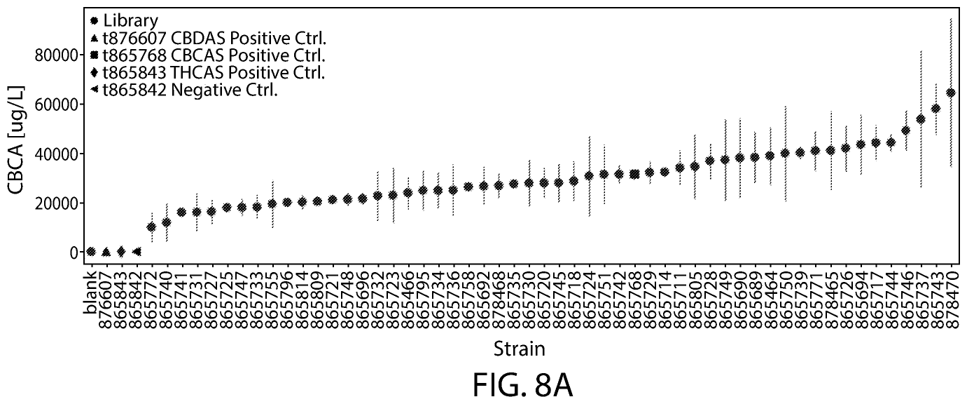

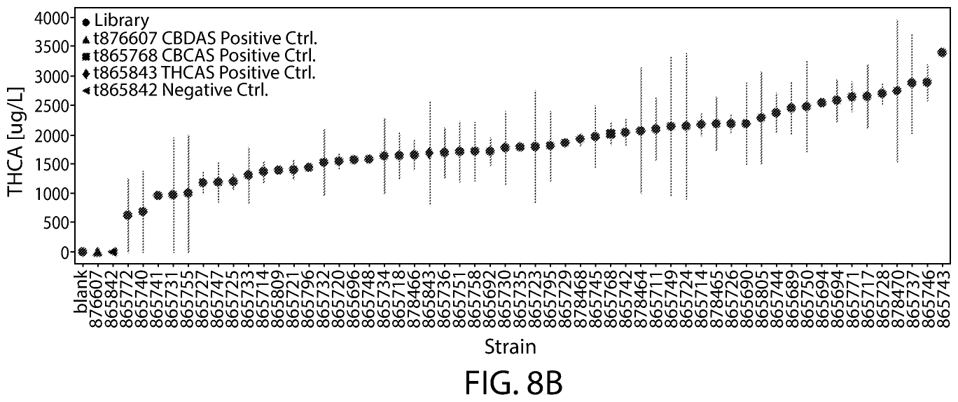

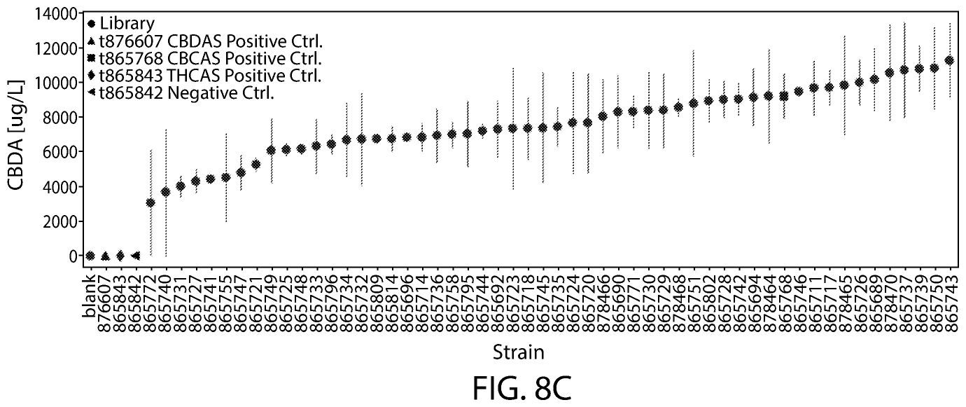

Of the 55 strains assessed in the secondary screen, 21 demonstrated a higher average CBCA titer than the A. niger positive control, including: strain t878470, which expresses a mutant version of A. niger CBCAS containing A57Q and G61A point mutations relative to SEQ ID NO: 27; strain t865743, which expresses a mutant version of A. niger CBCAS containing a V260M mutation relative to SEQ ID NO: 27; strain t865737, which expresses a mutant version of A. niger CBCAS containing a V62I mutation relative to SEQ ID NO: 27; strain t865746, which expresses a mutant version of A. niger CBCAS containing a V386A mutation relative to SEQ ID NO: 27; strain t865744, which expresses a mutant version of A. niger CBCAS containing a V260F mutation relative to SEQ ID NO: 27; strain t865717, which expresses a mutant version of A. niger CBCAS containing E112V and N122S point mutations relative to SEQ ID NO: 27; strain t865694, which expresses a mutant version of A. niger CBCAS containing A57E and I126A point mutations relative to SEQ ID NO: 27; strain t865726, which expresses a mutant version of A. niger CBCAS containing T33D and N257S point mutations relative to SEQ ID NO: 27; strain t878465, which expresses a mutant version of A. niger CBCAS containing N202S and P472A point mutations relative to SEQ ID NO: 27; strain t865771, which expresses a mutant version of A. niger CBCAS containing a D410N point mutation relative to SEQ ID NO: 27; strain t865739, which expresses a mutant version of A. niger CBCAS containing a R450K point mutation relative to SEQ ID NO: 27; strain t865750, which expresses a mutant version of A. niger CBCAS containing a S180T point mutation relative to SEQ ID NO: 27; strain t878464, which expresses a mutant version of A. niger CBCAS containing a R183T point mutation relative to SEQ ID NO: 27; strain t865689, which expresses a mutant version of A. niger CBCAS containing N122G and I126R point mutations relative to SEQ ID NO: 27; strain t865690, which expresses a mutant version of A. niger CBCAS containing N122A and I126T point mutations relative to SEQ ID NO: 27; strain t865749, which expresses a mutant version of A. niger CBCAS containing a Y71I point mutation relative to SEQ ID NO: 27; strain t865728, which expresses a mutant version of A. niger CBCAS containing H287R and A341S point mutations relative to SEQ ID NO: 27; strain t865805, which expresses a mutant version of A. niger CBCAS containing T55S and 1126T point mutations relative to SEQ ID NO: 27; strain t865711, which expresses a mutant version of A. niger CBCAS containing N122G and V398F point mutations relative to SEQ ID NO: 27; strain t865714, which expresses a mutant version of A. niger CBCAS containing a M394T point mutation relative to SEQ ID NO: 27; and strain t865729, which expresses a mutant version of A. niger CBCAS containing A57E and N131S point mutations relative to SEQ ID NO: 27. (FIG. 8A; Table 8.)

Surprisingly these 21 mutant CBCAS hits also demonstrated enhanced product specificity for CBCA. For example, the A. niger positive control produced a terminal cannabinoid product profile consisting of 73.74% CBCA, 21.55% CBDA, and 4.72% THCA, whereas certain CBCAS mutants were identified that produced more than 80% CBCA (80-83% CBCA, 13-14% CBDA, and 3-5% THCA).

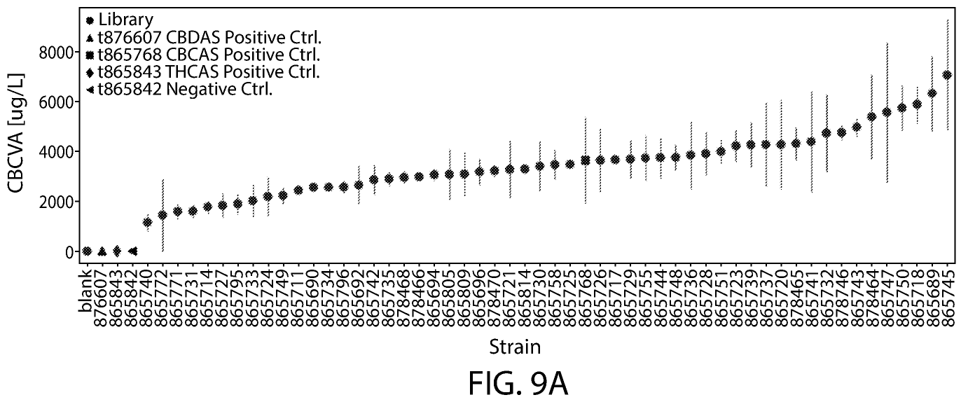

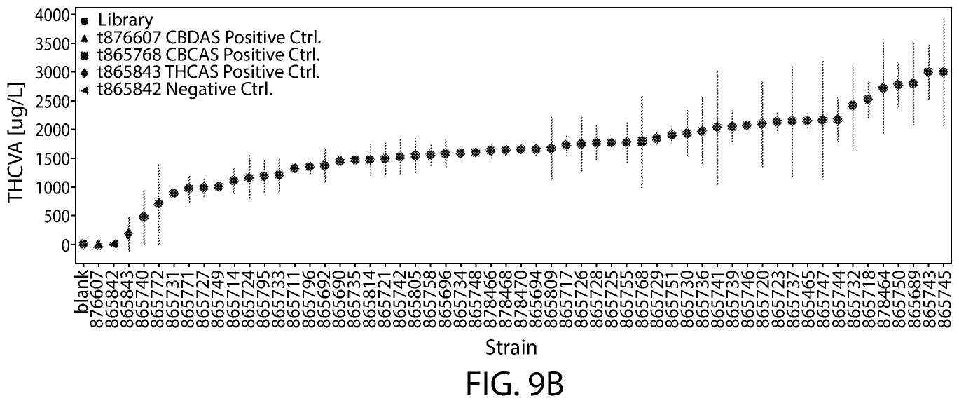

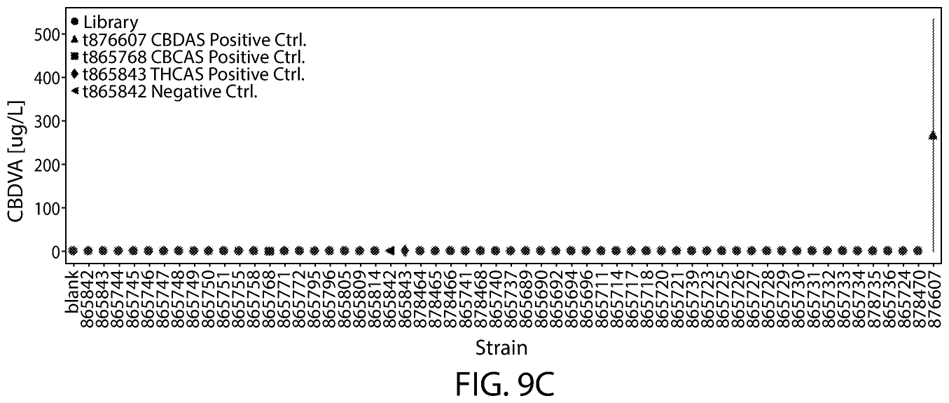

Of the 55 strains assessed in the secondary screen, 24 demonstrated a higher average CBCVA titer than the A. niger positive control, including: strain t865745, which expresses a mutant version of A. niger CBCAS containing a V63I point mutation relative to SEQ ID NO: 27; strain t865689, which expresses a mutant version of A. niger CBCAS containing N122G and I126R point mutations relative to SEQ ID NO: 27; strain t865718, which expresses a mutant version of A. niger CBCAS containing a P472R point mutation relative to SEQ ID NO: 27; strain t865750, which expresses a mutant version of A. niger CBCAS containing a S180T point mutation relative to SEQ ID NO: 27; strain t865747, which expresses a mutant version of A. niger CBCAS containing a V398A point mutation relative to SEQ ID NO: 27; strain t878464, which expresses a mutant version of A. niger CBCAS containing a R183T point mutation relative to SEQ ID NO: 27; strain t865743, which expresses a mutant version of A. niger CBCAS containing a V260M point mutation relative to SEQ ID NO: 27; strain t865746, which expresses a mutant version of A. niger CBCAS containing a V386A point mutation relative to SEQ ID NO: 27; strain t865732, which expresses a mutant version of A. niger CBCAS containing a H426Y point mutation relative to SEQ ID NO: 27; strain t865741, which expresses a mutant version of A. niger CBCAS containing a Y256M point mutation relative to SEQ ID NO: 27; strain t878465, which expresses a mutant version of A. niger CBCAS containing N202S and P472A point mutations relative to SEQ ID NO: 27; strain t865720, which expresses a mutant version of A. niger CBCAS containing N122G and I126K point mutations relative to SEQ ID NO: 27; strain t865737, which expresses a mutant version of A. niger CBCAS containing a V62I point mutation relative to SEQ ID NO: 27; strain t865739, which expresses a mutant version of A. niger CBCAS containing a R450K point mutation relative to SEQ ID NO: 27; strain t865723, which expresses a mutant version of A. niger CBCAS containing a Y129W point mutation relative to SEQ ID NO: 27; strain t865751, which expresses a mutant version of A. niger CBCAS containing a S423A point mutation relative to SEQ ID NO: 27; strain t865728, which expresses a mutant version of A. niger CBCAS containing H287R and A341S point mutations relative to SEQ ID NO: 27; strain t865736, which expresses a mutant version of A. niger CBCAS containing a N295S point mutation relative to SEQ ID NO: 27; strain t865748, which expresses a mutant version of A. niger CBCAS containing a Y39F point mutation relative to SEQ ID NO: 27; strain t865744, which expresses a mutant version of A. niger CBCAS containing a V260F point mutation relative to SEQ ID NO: 27; strain t865755, which expresses a mutant version of A. niger CBCAS containing a L392H point mutation relative to SEQ ID NO: 27; strain t865729, which expresses a mutant version of A. niger CBCAS containing A57E and N131S point mutations relative to SEQ ID NO: 27; strain t865717, which expresses a mutant version of A. niger CBCAS containing E112V and N122S point mutations relative to SEQ ID NO: 27; and strain t865726, which expresses a mutant version of A. niger CBCAS containing T33D and N257S point mutations relative to SEQ ID NO: 27. (FIG. 9A; Table 9.) Unlike for the hits identified on olivetolic acid, a shift in product profile was not observed among the terminal cannabinoids produced from divaric acid. Rather, this product profile was 67-70% CBCVA and 30-33% THCVA for both the A. niger control and the mutant hits. Surprisingly CBDVA was not observed among the products generated by the CBCAS candidates assessed in this screen.

Multiple library strains were observed to produce THCA and THCVA. Strain t865768, expressing the A. niger CBCAS produced a higher average THCA titer than the positive control THCAS strain (FIG. 8B; Table 8.). Additionally, 33 library strains expressing A. niger CBCAS mutants produced a higher average THCA titer than the positive control THCAS strain (FIG. 8B; Table 8.) Additionally, Strain t865768, expressing the A. niger CBCAS, and most of the tested library strains expressing A. niger CBCAS mutants produced more THCVA than the positive control THCAS strain (FIG. 9B; Table 9.)

Multiple library strains were also observed to produce CBDA. Strain t865768, expressing the A. niger CBCAS and most of the tested library strains expressing A. niger CBCAS mutants produced more CBDA than the positive control CBDAS strain (t876607), which expressed a Cannabis CBDAS. Consistent with previous reports (Luo et al. Nature, 2019 March; 567(7746):123-126), the Cannabis CBDAS has low to no activity in a S. cerevisiae host cell: (FIG. 8C; Table 8). No library strains tested were found to produce CBDVA (FIG. 9C; Table 9).

TABLE 8
CBCA, THCA, and CBDA titers from protein engineering of CBCAS candidate enzymes in S. cerevisiae

| | Strain type/ | | | | | | | | | |
|---|---|---|---|---|---|---|---|---|---|---|
| | Point mutations | Mean | Std Dev. | Mean | Std Dev. | Mean | Std Dev | | | |
| Strain | relative to | CBCA | CBCA | THCA | THCA | CBDA | CBDA | % | % | % |
| ID | SEQ ID NO: 27 | [μg/L] | [μg/L] | [μg/L] | [μg/L] | [μg/L] | [μg/L] | CBCA | THCA | CBDA |
| t865768 | A. niger CBCAS | 31539.55 | 2195.41 | 2016.94 | 224.36 | 9216.26 | 1477.71 | 73.74 | 4.72 | 21.55 |
| | Positive Control | | | | | | | | | |
| t865843 | THCAS | 0 | 0 | 1681.35 | 1025.75 | 0 | 0 | 0.00 | 100.00 | 0.00 |
| | Positive Control | | | | | | | | | |
| t876607 | CBDAS | 0 | 0 | 0 | 0 | 0 | 0 | 0.00 | 0.00 | 0.00 |
| | Positive Control | | | | | | | | | |
| t865842 | GFP | 0 | 0 | 0 | 0 | 0 | 0 | 0 | 0 | 0 |
| | Negative Control | | | | | | | | | |
| t878470 | Library/A57Q | 64502.70 | 42097.39 | 2739.24 | 1708.61 | 10538.52 | 3890.08 | 82.93 | 3.52 | 13.55 |
| | G61A | | | | | | | | | |
| t865743 | Library/V260M | 58061.40 | 14603.74 | 3389.10 | 103.61 | 11245.27 | 3070.56 | 79.87 | 4.66 | 15.47 |
| t865737 | Library/V62I | 53771.53 | 39388.63 | 2873.74 | 1195.99 | 10699.35 | 3847.33 | 79.85 | 4.27 | 15.89 |
| t865746 | Library/V386A | 49195.03 | 11206.00 | 2882.16 | 432.15 | 9456.09 | 136.04 | 79.95 | 4.68 | 15.37 |
| t865744 | Library/V260F | 44305.26 | 4660.79 | 2369.76 | 461.94 | 7187.94 | 595.36 | 82.26 | 4.40 | 13.34 |
| t865717 | Library/E112V | 44204.43 | 9829.72 | 2648.36 | 760.56 | 9698.33 | 1430.66 | 78.17 | 4.68 | 17.15 |
| | N122S | | | | | | | | | |
| t865694 | Library/A57E | 43506.37 | 17223.22 | 2579.57 | 496.08 | 9126.46 | 2305.93 | 78.80 | 4.67 | 16.53 |
| | I126A | | | | | | | | | |
| t865726 | Library/T33D | 41981.73 | 13073.05 | 2186.01 | 225.25 | 9985.90 | 1852.88 | 77.52 | 4.04 | 18.44 |
| | N257S | | | | | | | | | |
| t878465 | Library/N202S | 41094.07 | 22214.68 | 2184.77 | 642.72 | 9826.17 | 4038.53 | 77.38 | 4.11 | 18.50 |
| | P472A | | | | | | | | | |
| t865771 | Library/D410N | 40971.16 | 11253.32 | 2638.90 | 350.05 | 8309.68 | 1295.66 | 78.91 | 5.08 | 16.00 |
| t865739 | Library/R450K | 40214.21 | 3194.26 | 2538.89 | 45.95 | 10767.85 | 1830.53 | 75.14 | 4.74 | 20.12 |
| t865750 | Library/S180T | 39940.41 | 27152.74 | 2475.89 | 1084.67 | 10807.90 | 3325.20 | 75.04 | 4.65 | 20.31 |
| t878464 | Library/R183T | 38911.71 | 16555.91 | 2062.10 | 1512.86 | 9203.22 | 3865.34 | 77.55 | 4.11 | 18.34 |
| t865689 | Library/N122G | 38241.90 | 14591.60 | 2452.92 | 634.89 | 10157.65 | 2550.20 | 75.20 | 4.82 | 19.97 |
| | I126R | | | | | | | | | |
| t865690 | Library/N122A | 38065.26 | 22698.56 | 2186.36 | 977.36 | 8288.48 | 2915.70 | 78.42 | 4.50 | 17.08 |
| | I126T | | | | | | | | | |
| t865749 | Library/Y71I | 37290.06 | 23183.06 | 2140.74 | 1682.07 | 6071.00 | 2642.28 | 81.95 | 4.70 | 13.34 |
| t865728 | Library/H287R | 36875.12 | 10430.70 | 2692.19 | 245.09 | 8996.37 | 1486.17 | 75.93 | 5.54 | 18.52 |
| | A341S | | | | | | | | | |
| t865805 | Library/T55S | 34567.68 | 18187.95 | 2285.23 | 1105.08 | 8917.97 | 1733.41 | 75.52 | 4.99 | 19.48 |
| | I126T | | | | | | | | | |
| t865711 | Library/N122G | 33994.02 | 9784.58 | 2096.22 | 742.86 | 9666.71 | 2184.88 | 74.29 | 4.58 | 21.13 |
| | V398F | | | | | | | | | |
| t865714 | Library/M394T | 32311.22 | 2236.43 | 2172.74 | 264.10 | 6827.61 | 1091.58 | 78.21 | 5.26 | 16.53 |
| t865729 | Library/A57E | 32213.25 | 6584.57 | 1856.57 | 45.07 | 8392.06 | 3009.17 | 75.86 | 4.37 | 19.76 |
| | N131S | | | | | | | | | |
| t865742 | Library/E112T | 31427.04 | 4866.15 | 2036.13 | 312.97 | 9022.22 | 1377.35 | 73.97 | 4.79 | 21.24 |
| | N122G | | | | | | | | | |
| t865751 | Library/S423A | 31396.25 | 16606.59 | 1709.83 | 731.73 | 8775.99 | 4271.09 | 74.96 | 4.08 | 20.95 |
| t865724 | Library/T102N | 30758.44 | 22610.92 | 2146.00 | 1745.90 | 7663.56 | 4141.79 | 75.82 | 5.29 | 18.89 |
| | V114T | | | | | | | | | |
| t865718 | Library/P472R | 28669.67 | 11079.11 | 1640.20 | 565.38 | 7340.65 | 2480.50 | 76.15 | 4.36 | 19.50 |
| t865745 | Library/V63I | 27923.31 | 10753.63 | 1963.25 | 745.90 | 7360.14 | 4485.51 | 74.97 | 5.27 | 19.76 |
| t865720 | Library/N122G | 27895.13 | 8460.02 | 1543.57 | 181.34 | 7663.58 | 4035.53 | 75.18 | 4.16 | 20.66 |
| | I126K | | | | | | | | | |
| t865730 | Library/N202G | 27874.45 | 13102.29 | 1771.94 | 885.27 | 8385.38 | 3086.02 | 73.29 | 4.66 | 22.05 |
| | H466N | | | | | | | | | |
| t865735 | Library/T446P | 27519.94 | 94.67 | 1783.72 | 69.10 | 7436.20 | 1564.24 | 74.90 | 4.85 | 20.24 |
| | H466N | | | | | | | | | |
| t878468 | Library/A57E | 26823.18 | 6838.86 | 1922.88 | 150.52 | 8556.40 | 711.58 | 71.91 | 5.15 | 22.94 |
| | T102Q | | | | | | | | | |
| t865692 | Library/A57E | 26625.20 | 10692.03 | 1712.61 | 326.02 | 7293.40 | 2324.07 | 74.72 | 4.81 | 20.47 |
| | T102S | | | | | | | | | |
| t865758 | Library/E456A | 26316.76 | 980.84 | 1712.53 | 710.87 | 6998.62 | 1073.23 | 75.13 | 4.89 | 19.98 |
| | H466N | | | | | | | | | |
| t865736 | Library/N295S | 24918.92 | 14722.56 | 1690.72 | 597.82 | 6931.79 | 2177.76 | 74.29 | 5.04 | 20.67 |
| t865734 | Library/A57E | 24880.40 | 10047.91 | 1632.06 | 905.04 | 6677.34 | 3006.55 | 74.96 | 4.92 | 20.12 |
| | G61A | | | | | | | | | |
| t865795 | Library/F262I | 24874.10 | 11028.22 | 1807.99 | 837.97 | 7028.82 | 2643.85 | 73.79 | 5.36 | 20.85 |
| t878466 | Library/Q161K | 23882.09 | 8907.36 | 1649.00 | 361.30 | 8030.74 | 3022.45 | 71.16 | 4.91 | 23.93 |
| t865723 | Library/Y129W | 22893.08 | 15795.28 | 1788.74 | 1346.84 | 7334.92 | 4905.12 | 71.50 | 5.59 | 22.91 |
| t865732 | Library/H426Y | 22672.81 | 14284.27 | 1523.15 | 807.78 | 6720.79 | 3752.27 | 73.34 | 4.93 | 21.74 |
| t865696 | Library/N122G | 21496.89 | 3186.39 | 1567.45 | 49.69 | 6820.56 | 218.82 | 71.93 | 5.24 | 22.82 |
| | G469S | | | | | | | | | |
| t865748 | Library/Y39F | 21260.42 | 3672.95 | 1575.92 | 19.15 | 6161.32 | 386.21 | 73.32 | 5.43 | 21.25 |
| t865721 | Library/Y256F | 21099.41 | 1743.97 | 1396.80 | 220.29 | 5260.35 | 544.40 | 76.02 | 5.03 | 18.95 |
| t865809 | Library/N122G | 20413.40 | 971.41 | 1390.36 | 1.49 | 6738.75 | 219.63 | 71.52 | 4.87 | 23.61 |
| | I126D | | | | | | | | | |
| t865814 | Library/D280N | 20192.93 | 3941.32 | 1367.48 | 249.49 | 6751.44 | 1040.07 | 71.32 | 4.83 | 23.85 |
| t865796 | Library/L458W | 19975.57 | 898.91 | 1436.09 | 62.66 | 6427.56 | 744.81 | 71.75 | 5.16 | 23.09 |
| t865755 | Library/L392H | 19432.21 | 13347.17 | 1001.22 | 1415.94 | 4507.57 | 3615.50 | 77.91 | 4.01 | 18.07 |
| t865733 | Library/V398T | 18070.99 | 6701.89 | 1307.71 | 675.32 | 6320.33 | 2190.16 | 70.32 | 5.09 | 24.59 |
| | H466N | | | | | | | | | |
| t865747 | Library/V398A | 18021.77 | 4484.44 | 1188.04 | 471.71 | 4783.24 | 1417.67 | 75.11 | 4.95 | 19.94 |
| t865725 | Library/N122G | 17948.56 | 2644.64 | 1197.84 | 184.04 | 6120.73 | 541.65 | 71.04 | 4.74 | 24.22 |
| | I126A | | | | | | | | | |
| t865727 | Library/H353A | 16276.39 | 7210.82 | 1177.87 | 281.84 | 4297.38 | 938.33 | 74.83 | 5.42 | 19.76 |
| | E456A | | | | | | | | | |
| t865731 | Library/V25A | 16059.53 | 10389.40 | 971.80 | 1374.33 | 4006.74 | 838.84 | 76.34 | 4.62 | 19.05 |
| | L43I | | | | | | | | | |
| t865741 | Library/Y256M | 15982.40 | 1243.85 | 957.33 | 44.81 | 4419.64 | 132.81 | 74.83 | 4.48 | 20.69 |
| t865740 | Library/N122E | 11837.41 | 10494.74 | 685.53 | 969.48 | 3671.56 | 5192.38 | 73.10 | 4.23 | 22.67 |
| | V398L | | | | | | | | | |
| t865772 | Library/D35A | 9992.22 | 8522.88 | 622.72 | 880.66 | 3051.02 | 4314.80 | 73.12 | 4.56 | 22.33 |

TABLE 9
CBCVA, THCVA, and CBDVA titers from protein engineering of CBCAS candidate enzymes in S. cerevisiae

| | Strain type/ | | | | | | | | | |
|---|---|---|---|---|---|---|---|---|---|---|
| | Point mutations | Mean | Std Dev. | Mean | Std Dev. | Mean | Std Dev. | | | |
| Strain | relative to | CBCVA | CBCVA | THCVA | THCVA | CBDVA | CBDVA | % | % | % |
| ID | SEQ ID NO: 27 | [μg/L] | [μg/L] | [μg/L] | [μg/L] | [μg/L] | [μg/L] | CBCVA | THCVA | CBDVA |
| t865768 | A. niger CBCAS | 3642.91 | 1964.14 | 1788.13 | 915.18 | 0.00 | 0.00 | 67.08 | 32.92 | 0.00 |
| | Positive Control | | | | | | | | | |
| t865843 | THCAS | 0 | 0 | 175.02 | 350.06 | 0 | 0 | 0.00 | 100.00 | 0.00 |
| | Positive Control | | | | | | | | | |
| t876607 | CBDAS | 0 | 0 | 0 | 0 | 265.53 | 308.55 | 0.00 | 0.00 | 100.00 |
| | Positive Control | | | | | | | | | |
| t865842 | GFP | 0 | 0 | 0 | 0 | 0 | 0 | 0.00 | 0.00 | 0.00 |
| | Negative Control | | | | | | | | | |
| t865745 | Library/V63I | 7068.26 | 3144.76 | 2991.05 | 1315.58 | 0.00 | 0.00 | 70.27 | 29.73 | 0.00 |
| t865689 | Library/N122G | 6333.32 | 2138.98 | 2791.18 | 1019.00 | 0.00 | 0.00 | 69.41 | 30.59 | 0.00 |
| | I126R | | | | | | | | | |
| t865718 | Library/P472R | 5888.44 | 1041.48 | 2516.89 | 454.55 | 0.00 | 0.00 | 70.06 | 29.94 | 0.00 |
| t865750 | Library/S180T | 5745.78 | 1265.89 | 2770.13 | 539.05 | 0.00 | 0.00 | 67.47 | 32.53 | 0.00 |
| t865747 | Library/V398A | 5571.51 | 3965.98 | 2154.32 | 1459.29 | 0.00 | 0.00 | 72.12 | 27.88 | 0.00 |
| t878464 | Library/R183T | 5383.16 | 2382.21 | 2710.86 | 1113.16 | 0.00 | 0.00 | 66.51 | 33.49 | 0.00 |
| t865743 | Library/V260M | 4972.60 | 518.55 | 2989.22 | 662.39 | 0.00 | 0.00 | 62.46 | 37.54 | 0.00 |
| t865746 | Library/V386A | 4751.98 | 396.86 | 2061.14 | 73.01 | 0.00 | 0.00 | 69.75 | 30.25 | 0.00 |
| t865732 | Library/H426Y | 4734.85 | 2171.13 | 2408.74 | 994.86 | 0.00 | 0.00 | 66.28 | 33.72 | 0.00 |
| t865741 | Library/Y256M | 4388.54 | 2838.45 | 2033.77 | 1407.31 | 0.00 | 0.00 | 68.33 | 31.67 | 0.00 |
| t878465 | Library/N202S | 4314.23 | 902.00 | 2144.09 | 215.55 | 0.00 | 0.00 | 66.80 | 33.20 | 0.00 |
| | P472A | | | | | | | | | |
| t865720 | Library/N122G | 4276.65 | 2499.99 | 2090.51 | 1046.91 | 0.00 | 0.00 | 67.17 | 32.83 | 0.00 |
| | I126K | | | | | | | | | |
| t865737 | Library/V62I | 4271.01 | 2381.10 | 2136.23 | 1383.65 | 0.00 | 0.00 | 66.66 | 33.34 | 0.00 |
| t865739 | Library/R450K | 4265.42 | 1259.39 | 2039.44 | 391.72 | 0.00 | 0.00 | 67.65 | 32.35 | 0.00 |
| t865723 | Library/Y129W | 4223.36 | 891.21 | 2125.21 | 229.49 | 0.00 | 0.00 | 66.52 | 33.48 | 0.00 |
| t865751 | Library/S423A | 3998.68 | 626.37 | 1894.39 | 203.65 | 0.00 | 0.00 | 67.85 | 32.15 | 0.00 |
| t865728 | Library/H287R | 3907.72 | 1195.24 | 1759.32 | 427.70 | 0.00 | 0.00 | 68.96 | 31.04 | 0.00 |
| | A341S | | | | | | | | | |
| t865736 | Library/N295S | 3847.79 | 1905.25 | 1963.40 | 832.27 | 0.00 | 0.00 | 66.21 | 33.79 | 0.00 |
| t865748 | Library/Y39F | 3759.89 | 702.53 | 1591.63 | 75.81 | 0.00 | 0.00 | 70.26 | 29.74 | 0.00 |
| t865744 | Library/V260F | 3752.20 | 1126.84 | 2162.61 | 542.42 | 0.00 | 0.00 | 63.44 | 36.56 | 0.00 |
| t865755 | Library/L392H | 3729.91 | 1298.74 | 1768.56 | 500.12 | 0.00 | 0.00 | 67.84 | 32.16 | 0.00 |
| t865729 | Library/A57E | 3685.70 | 1033.39 | 1839.38 | 172.75 | 0.00 | 0.00 | 66.71 | 33.29 | 0.00 |
| | N131S | | | | | | | | | |
| t865717 | Library/E112V | 3668.51 | 73.58 | 1721.38 | 239.45 | 0.00 | 0.00 | 68.06 | 31.94 | 0.00 |
| | N122S | | | | | | | | | |
| t865726 | Library/T33D | 3644.48 | 1808.16 | 1740.51 | 652.00 | 0.00 | 0.00 | 67.68 | 32.32 | 0.00 |
| | N257S | | | | | | | | | |
| t865725 | Library/N122G | 3484.40 | 192.25 | 1759.92 | 87.91 | 0.00 | 0.00 | 66.44 | 33.56 | 0.00 |
| | I126A | | | | | | | | | |
| t865758 | Library/E456A | 3465.87 | 822.25 | 1548.62 | 269.59 | 0.00 | 0.00 | 69.12 | 30.88 | 0.00 |
| | H466N | | | | | | | | | |
| t865730 | Library/N202G | 3406.05 | 1412.56 | 1922.88 | 570.78 | 0.00 | 0.00 | 63.92 | 36.08 | 0.00 |
| | H466N | | | | | | | | | |
| t865814 | Library/D280N | 3290.24 | 101.52 | 1468.01 | 404.74 | 0.00 | 0.00 | 69.15 | 30.85 | 0.00 |
| t865721 | Library/Y256F | 3281.34 | 1586.08 | 1482.54 | 379.24 | 0.00 | 0.00 | 68.88 | 31.12 | 0.00 |
| t878470 | Library/A57Q | 3226.77 | 314.59 | 1646.54 | 2.72 | 0.00 | 0.00 | 66.21 | 33.79 | 0.00 |
| | G61A | | | | | | | | | |
| t865696 | Library/N122G | 3184.90 | 726.92 | 1570.38 | 334.49 | 0.00 | 0.00 | 66.98 | 33.02 | 0.00 |
| | G469S | | | | | | | | | |
| t865809 | Library/N122G | 3093.25 | 1227.36 | 1662.32 | 761.41 | 0.00 | 0.00 | 65.04 | 34.96 | 0.00 |
| | I126D | | | | | | | | | |
| t865805 | Library/T55S | 3077.84 | 1412.48 | 1538.55 | 421.80 | 0.00 | 0.00 | 66.67 | 33.33 | 0.00 |
| | I126T | | | | | | | | | |
| t865694 | Library/A57E | 3069.69 | 294.21 | 1647.98 | 140.28 | 0.00 | 0.00 | 65.07 | 34.93 | 0.00 |
| | I126A | | | | | | | | | |
| t878466 | Library/Q161K | 2985.03 | 62.33 | 1623.40 | 158.35 | 0.00 | 0.00 | 64.77 | 35.23 | 0.00 |
| t878468 | Library/A57E | 2954.90 | 335.09 | 1628.92 | 115.38 | 0.00 | 0.00 | 64.46 | 35.54 | 0.00 |
| | T102Q | | | | | | | | | |
| t865735 | Library/T446P | 2900.35 | 358.56 | 1459.81 | 7.23 | 0.00 | 0.00 | 66.52 | 33.48 | 0.00 |
| | H466N | | | | | | | | | |
| t865742 | Library/E112T | 2864.87 | 812.38 | 1514.38 | 416.60 | 0.00 | 0.00 | 65.42 | 34.58 | 0.00 |
| | N122G | | | | | | | | | |
| t865692 | Library/A57E | 2649.69 | 1065.13 | 1366.19 | 421.31 | 0.00 | 0.00 | 65.98 | 34.02 | 0.00 |
| | T102S | | | | | | | | | |
| t865796 | Library/L458W | 2570.89 | 328.86 | 1344.71 | 162.60 | 0.00 | 0.00 | 65.66 | 34.34 | 0.00 |
| t865734 | Library/A57E | 2566.05 | 177.20 | 1577.56 | 95.34 | 0.00 | 0.00 | 61.93 | 38.07 | 0.00 |
| | G61A | | | | | | | | | |
| t865690 | Library/N122A | 2557.72 | 165.88 | 1441.19 | 90.67 | 0.00 | 0.00 | 63.96 | 36.04 | 0.00 |
| | I126T | | | | | | | | | |
| t865711 | Library/N122G | 2442.93 | 95.92 | 1315.45 | 53.48 | 0.00 | 0.00 | 65.00 | 35.00 | 0.00 |
| | V398F | | | | | | | | | |
| t865749 | Library/Y71I | 2230.06 | 429.99 | 997.07 | 40.32 | 0.00 | 0.00 | 69.10 | 30.90 | 0.00 |
| t865724 | Library/T102N | 2190.11 | 1124.38 | 1153.25 | 541.45 | 0.00 | 0.00 | 65.51 | 34.49 | 0.00 |
| | V114T | | | | | | | | | |
| t865733 | Library/V398T | 2023.09 | 907.28 | 1202.96 | 424.48 | 0.00 | 0.00 | 62.71 | 37.29 | 0.00 |
| | H466N | | | | | | | | | |
| t865795 | Library/F262I | 1897.16 | 554.17 | 1181.24 | 377.67 | 0.00 | 0.00 | 61.63 | 38.37 | 0.00 |
| t865727 | Library/H353A | 1829.32 | 696.52 | 981.31 | 223.32 | 0.00 | 0.00 | 65.09 | 34.91 | 0.00 |
| | E456A | | | | | | | | | |
| t865714 | Library/M394T | 1775.08 | 353.96 | 1101.76 | 302.87 | 0.00 | 0.00 | 61.70 | 38.30 | 0.00 |
| t865731 | Library/V25A | 1605.94 | 368.12 | 885.33 | 26.61 | 0.00 | 0.00 | 64.46 | 35.54 | 0.00 |
| | L43I | | | | | | | | | |
| t865771 | Library/D410N | 1592.02 | 388.99 | 968.82 | 349.88 | 0.00 | 0.00 | 62.17 | 37.83 | 0.00 |
| t865772 | Library/D35A | 1441.55 | 2038.66 | 702.24 | 993.12 | 0.00 | 0.00 | 67.24 | 32.76 | 0.00 |
| t865740 | Library/N122E | 1153.83 | 483.47 | 469.98 | 664.66 | 0.00 | 0.00 | 71.06 | 28.94 | 0.00 |
| | V398L | | | | | | | | | |

### Example 3: High-Throughput Screen to Identify Metagenomic Cannabichromenic Acid Synthases (CRCASs)

To our knowledge the CBCAS from A. niger identified in Example 1 represents the first enzyme possessing this activity to be discovered outside of the Cannabis genus. To explore whether other putative CBCASs may exist in the broader metagenome, a library of 1072 candidate CBCAS genes was designed using the A. niger CBCAS enzyme identified in Example 1 as a reference. Protein sequences were recoded in silico for expression in S. cerevisiae and synthesized in the integrative yeast expression vector shown in FIG. 5. Each candidate enzyme expression construct was transformed into an S. cerevisiae CEN.PK strain that also expressed a prenyltransferase enzyme capable of catalyzing reaction R⁴ in FIG. 2. Strain t616313, expressing GFP, was included in the library screen as a negative control for enzyme activity. Strain t807925, expressing the A. niger enzyme identified in Example 1, was included in the library screen as a positive control for enzyme activity. All candidate enzymes in the library, as well as the enzyme expressed by positive control strain t807925, included an N-terminal MFalpha2 signal peptide (SEQ ID NO: 16) (with a methionine residue added at the N-terminus of the MFalpha2 signal peptide) and a C-terminal HDEL signal peptide (SEQ ID NO: 17).

The library of candidate CBCAS enzymes was assayed for activity in a primary high-throughput screen using the assay described in Example 1. Production of CBCA, THCA, and/or CBDA in the samples was quantified via LC-MS.

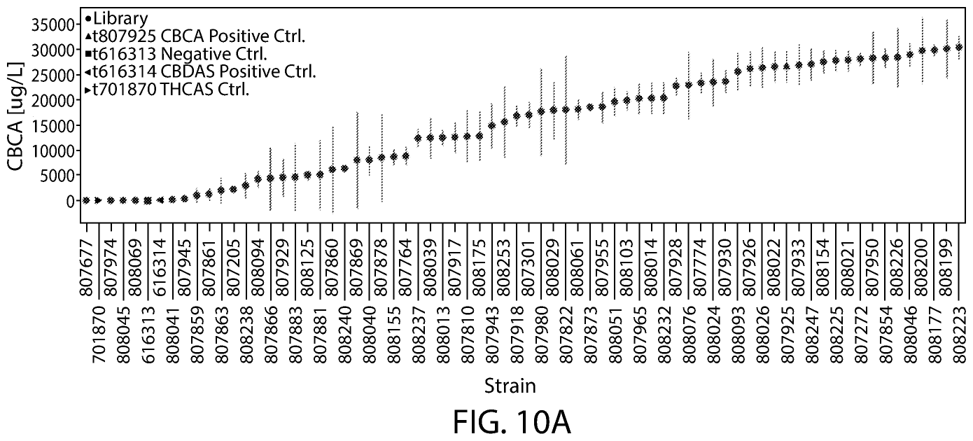

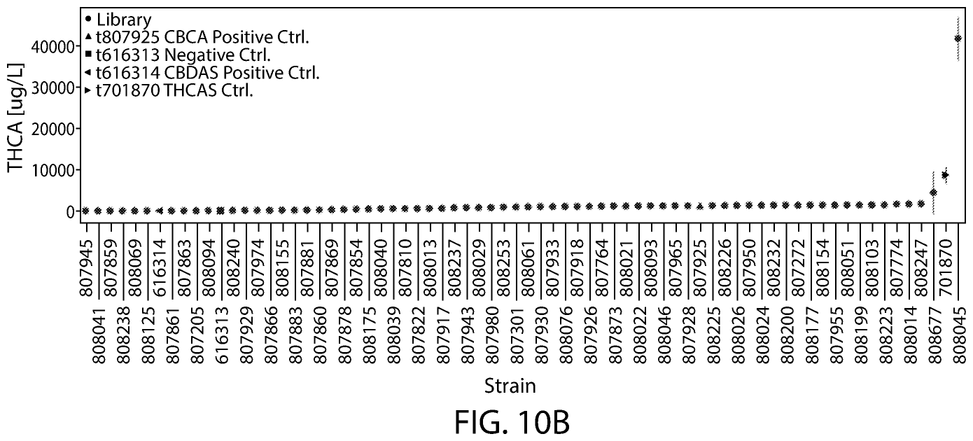

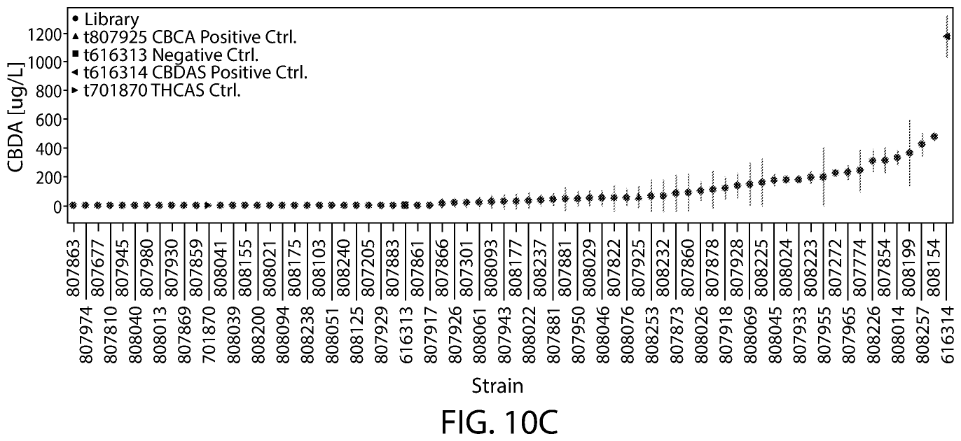

Based on results of the primary screen, 70 strains were carried forward to a secondary screen to confirm activity observed in the primary screen. The experimental protocol for the secondary screen was identical to the primary screen, except that additional technical replicates were included per strain, and replicate production cultures for each strain were separately fed 1 mM olivetolic acid or 1 mM divaric acid. All strains were screened in quadruplicate (FIGS. 10A-10C, Tables 10 and 11). Strain IDs and their corresponding sequences are shown in Table 15.

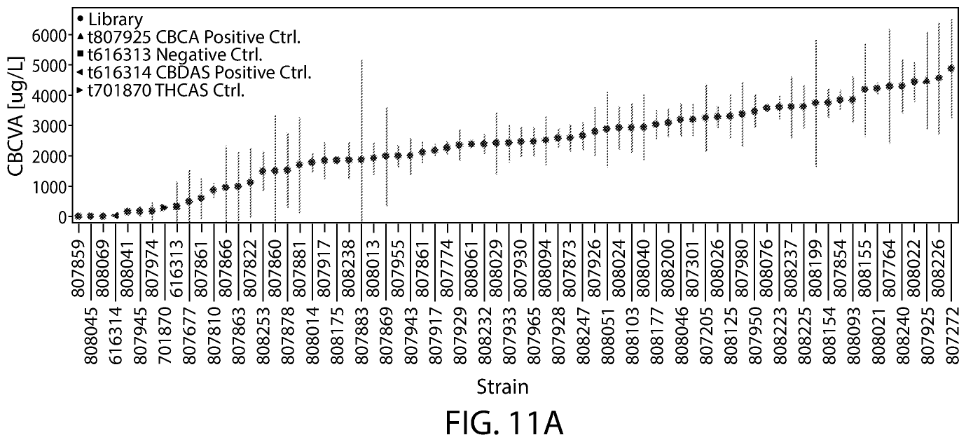

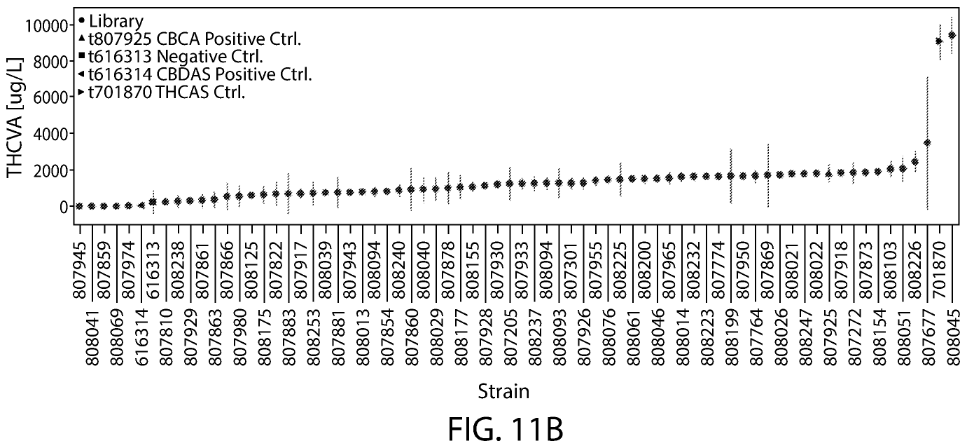

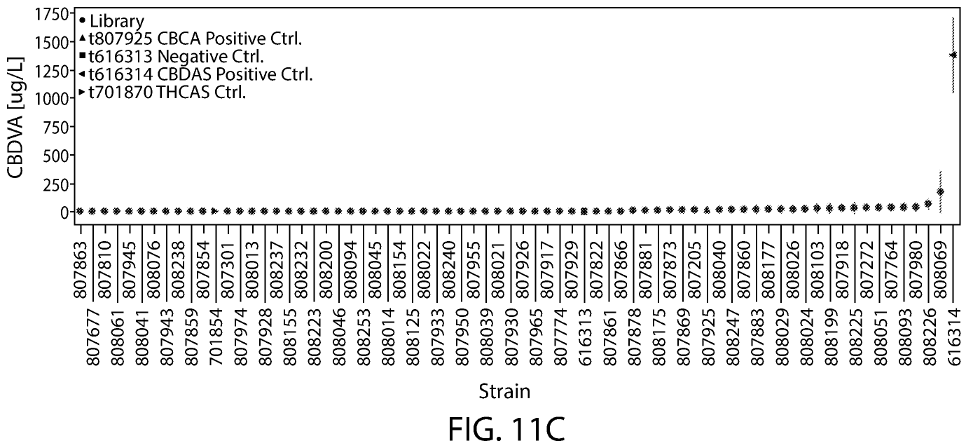

These results surprisingly identified multiple strains that are capable of producing CBCA and/or CBCVA. Specifically, 17 strains produced amounts of CBCA comparable to amounts produced by the positive control (corresponding to a mean CBCA titer at least within 1 standard deviation of the mean CBCA titer of strain t807925) while 2 strains (t808223 and t808199) produced CBCA at a titer of more than 1 standard deviation of the mean CBCA titer of strain t807925 (FIG. 10A). 28 strains demonstrated comparable CBCVAS activity to the positive control (FIG. 11A). Of these 17 strains, multiple strains, including: t807854—SEQ ID NO: 112, t807933—SEQ ID NO: 130, t808225—SEQ ID NO: 166, t808026—SEQ ID NO: 144, and t8082001—SEQ ID NO: 164 produced a terminal cannabinoid product profile with a higher percentage of CBCA than the A. niger positive control, with 1 strain (t807854—SEQ ID NO: 112) producing terminal cannabinoid products with a profile of over 97% CBCA.

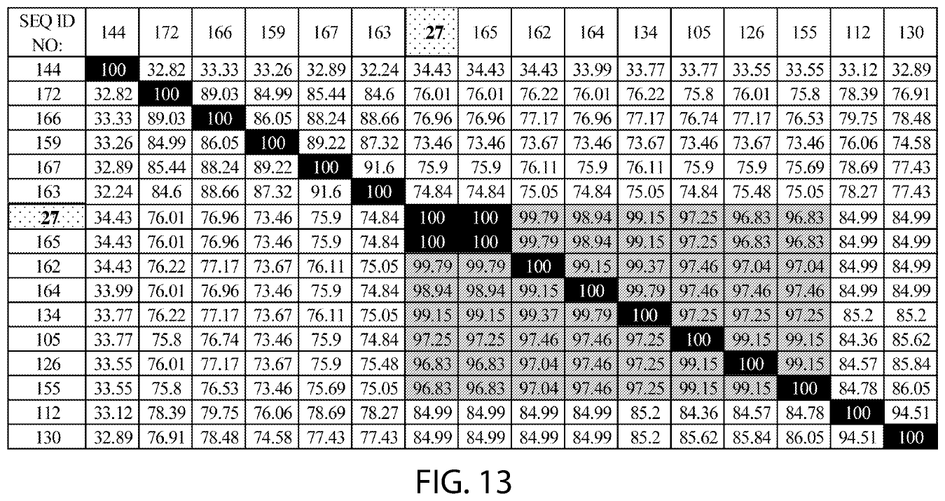

A subset of candidate CBCASs was identified that exhibited >95% sequence identity to the A. niger CBCAS identified in Example 1 (FIG. 13).

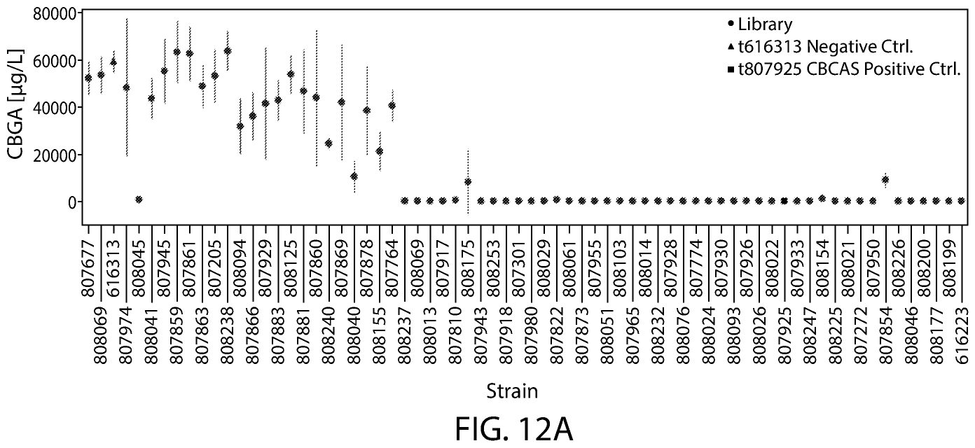

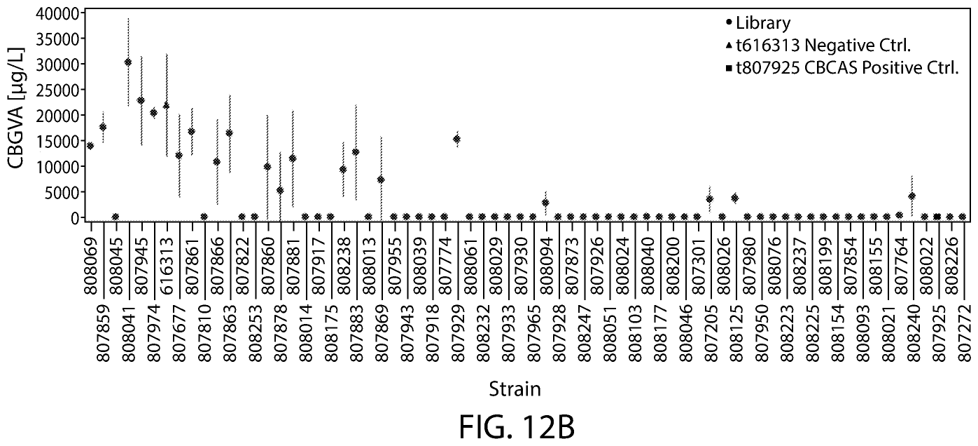

It was observed that several strains that produced CBCA and/or CBCVA completely exhausted their respective substrate (e.g., CBGA or CBGVA) (FIGS. 12A-12B, Table 12). Accordingly, while multiple strains were identified that are capable of producing CBCA and/or CBCVA, the observed substrate exhaustion precludes effective ranking between the strains based on production of CBCA.

TABLE 10
CBCA, THCA, and CBDA titers from metagenomic screening of CBCAS candidate enzymes in S. cerevisiae

| | | | Mean | Std Dev. | Mean | Std Dev. | Mean | Std Dev | | | |
|---|---|---|---|---|---|---|---|---|---|---|---|
| Strain | Strain | TS SEQ | CBCA | CBCA | THCA | THCA | CBDA | CBDA | % | % | % |
| ID | Type | ID NO* | [μg/L] | [μg/L] | [μg/L] | [μg/L] | [μg/L] | [μg/L] | CBCA | THCA | CBDA |
| t807925 | A. niger CBCAS | 27 | 26702.23 | 3170.88 | 1248.46 | 146.74 | 59.53 | 81.81 | 95.33 | 4.46 | 0.21 |
| | Positive Control | | | | | | | | | | |
| t616313 | GFP | — | 0 | 0 | 103.88 | 293.83 | 0 | 0 | 0.00 | 100.00 | 0.00 |
| | Negative Control | | | | | | | | | | |
| t616314 | CBDAS | — | 60.45 | 170.99 | 0.00 | 0.00 | 1170.28 | 150.50 | 4.91 | 0.00 | 95.09 |
| | Positive Control | | | | | | | | | | |
| t701870 | THCAS | — | 0 | 0 | 8608.03 | 1979.341 | 0 | 0 | 0.00 | 100.00 | 0.00 |
| | Positive control | | | | | | | | | | |
| t807205 | Library | 104 | 2190.95 | 195.13 | 28.98 | 57.97 | 0.00 | 0.00 | 98.69 | 1.31 | 0.00 |
| t807272 | Library | 105 | 28089.30 | 1594.65 | 1372.35 | 166.84 | 222.98 | 12.56 | 94.63 | 4.62 | 0.75 |
| t807301 | Library | 106 | 16894.33 | 3008.12 | 934.75 | 231.20 | 19.38 | 38.75 | 94.65 | 5.24 | 0.11 |
| t807677 | Library | 107 | 0.00 | 0.00 | 4464.43 | 5549.24 | 0.00 | 0.00 | 0.00 | 100.00 | 0.00 |
| t807764 | Library | 108 | 8745.39 | 2597.12 | 1145.59 | 313.94 | 41.75 | 59.04 | 88.05 | 11.53 | 0.42 |
| t807774 | Library | 109 | 23257.40 | 2358.46 | 1638.75 | 138.49 | 239.69 | 165.44 | 92.53 | 6.52 | 0.95 |
| t807810 | Library | 110 | 12633.04 | 5930.64 | 547.64 | 263.95 | 0.00 | 0.00 | 95.85 | 4.15 | 0.00 |
| t807822 | Library | 111 | 17911.95 | 12548.56 | 548.59 | 402.89 | 52.68 | 105.37 | 96.75 | 2.96 | 0.28 |
| t807854 | Library | 112 | 28295.73 | 2137.45 | 389.02 | 29.38 | 309.68 | 99.97 | 97.59 | 1.34 | 1.07 |
| t807859 | Library | 113 | 979.04 | 1622.16 | 0.00 | 0.00 | 0.00 | 0.00 | 100.00 | 0.00 | 0.00 |
| t807860 | Library | 114 | 6059.67 | 9428.46 | 242.75 | 379.93 | 88.08 | 136.55 | 94.82 | 3.80 | 1.38 |
| t807861 | Library | 115 | 1263.83 | 1366.48 | 0.00 | 0.00 | 0.00 | 0.00 | 100.00 | 0.00 | 0.00 |
| t807863 | Library | 116 | 2009.31 | 2653.08 | 17.48 | 49.43 | 0.00 | 0.00 | 99.14 | 0.86 | 0.00 |
| t807866 | Library | 117 | 4331.01 | 6721.32 | 137.76 | 213.75 | 14.26 | 34.94 | 96.61 | 3.07 | 0.32 |
| t807869 | Library | 118 | 7944.59 | 10155.04 | 281.60 | 464.93 | 0.00 | 0.00 | 96.58 | 3.42 | 0.00 |
| t807873 | Library | 120 | 18433.59 | 705.23 | 1175.62 | 144.22 | 85.27 | 135.97 | 93.60 | 5.97 | 0.43 |
| t807878 | Library | 121 | 8442.32 | 9157.09 | 315.30 | 360.65 | 110.64 | 136.38 | 95.20 | 3.56 | 1.25 |
| t807881 | Library | 122 | 5077.61 | 7218.42 | 192.96 | 320.81 | 44.99 | 84.48 | 95.52 | 3.63 | 0.85 |
| t807883 | Library | 123 | 4606.20 | 7284.45 | 181.54 | 281.46 | 0.00 | 0.00 | 96.21 | 3.79 | 0.00 |
| t807917 | Library | 124 | 12476.94 | 3431.70 | 600.43 | 166.06 | 0.00 | 0.00 | 95.41 | 4.59 | 0.00 |
| t807918 | Library | 125 | 16735.84 | 2219.45 | 1065.19 | 112.89 | 119.68 | 87.28 | 93.39 | 5.94 | 0.67 |
| t807926 | Library | 126 | 26139.45 | 4019.03 | 1101.73 | 185.88 | 18.67 | 37.34 | 95.89 | 4.04 | 0.07 |
| t807928 | Library | 127 | 22647.99 | 1997.52 | 1240.90 | 218.30 | 136.60 | 95.46 | 94.27 | 5.16 | 0.57 |
| t807929 | Library | 128 | 4498.23 | 4252.58 | 119.42 | 238.83 | 0.00 | 0.00 | 97.41 | 2.59 | 0.00 |
| t807930 | Library | 129 | 23580.19 | 2507.70 | 1014.24 | 166.36 | 0.00 | 0.00 | 95.88 | 4.12 | 0.00 |
| t807933 | Library | 130 | 26844.72 | 4730.41 | 1040.73 | 129.25 | 178.27 | 23.02 | 95.66 | 3.71 | 0.64 |
| t807943 | Library | 131 | 14764.41 | 5042.77 | 781.93 | 369.01 | 27.46 | 54.92 | 94.80 | 5.02 | 0.18 |
| t807945 | Library | 132 | 333.08 | 385.97 | 0.00 | 0.00 | 0.00 | 0.00 | 100.00 | 0.00 | 0.00 |
| t807950 | Library | 134 | 28235.47 | 5978.18 | 1351.19 | 306.97 | 46.31 | 57.36 | 95.28 | 4.56 | 0.16 |
| t807955 | Library | 135 | 18487.09 | 3459.56 | 1410.52 | 211.16 | 195.38 | 228.38 | 92.01 | 7.02 | 0.97 |
| t807965 | Library | 136 | 20155.49 | 3425.87 | 1240.06 | 94.02 | 227.49 | 51.37 | 93.21 | 5.73 | 1.05 |
| t807974 | Library | 137 | 0.00 | 0.00 | 136.24 | 191.02 | 0.00 | 0.00 | 0.00 | 100.00 | 0.00 |
| t807980 | Library | 138 | 17555.95 | 10045.15 | 806.09 | 358.39 | 0.00 | 0.00 | 95.61 | 4.39 | 0.00 |
| t808013 | Library | 139 | 12365.50 | 1671.57 | 568.09 | 55.87 | 0.00 | 0.00 | 95.61 | 4.39 | 0.00 |
| t808014 | Library | 140 | 20225.49 | 3555.31 | 1665.44 | 419.41 | 327.63 | 58.07 | 91.03 | 7.50 | 1.47 |
| t808021 | Library | 141 | 27854.09 | 2394.77 | 1180.40 | 174.07 | 0.00 | 0.00 | 95.93 | 4.07 | 0.00 |
| t808022 | Library | 142 | 26546.08 | 3396.30 | 1197.03 | 149.25 | 33.24 | 66.47 | 95.57 | 4.31 | 0.12 |
| t808024 | Library | 143 | 23438.63 | 5403.63 | 1364.49 | 198.52 | 176.59 | 35.94 | 93.83 | 5.46 | 0.71 |
| t808026 | Library | 144 | 26319.85 | 4554.96 | 1317.85 | 203.24 | 101.58 | 74.46 | 94.88 | 4.75 | 0.37 |
| t808029 | Library | 145 | 17841.91 | 6669.16 | 781.98 | 293.41 | 51.99 | 60.16 | 95.53 | 4.19 | 0.28 |
| t808039 | Library | 146 | 12361.14 | 4562.70 | 543.01 | 180.84 | 0.00 | 0.00 | 95.79 | 4.21 | 0.00 |
| t808040 | Library | 147 | 7960.31 | 3266.01 | 500.18 | 196.90 | 0.00 | 0.00 | 94.09 | 5.91 | 0.00 |
| t808041 | Library | 148 | 166.10 | 332.19 | 0.00 | 0.00 | 0.00 | 0.00 | 100.00 | 0.00 | 0.00 |
| t808045 | Library | 149 | 0.00 | 0.00 | 41807.82 | 5921.89 | 173.71 | 45.77 | 0.00 | 99.59 | 0.41 |
| t808046 | Library | 150 | 28934.98 | 3189.39 | 1236.39 | 16.70 | 52.38 | 74.08 | 95.74 | 4.09 | 0.17 |
| t808051 | Library | 151 | 19541.60 | 3262.21 | 1412.60 | 204.83 | 0.00 | 0.00 | 93.26 | 6.74 | 0.00 |
| t808061 | Library | 152 | 18022.20 | 2272.19 | 975.95 | 149.37 | 22.10 | 44.21 | 94.75 | 5.13 | 0.12 |
| t808069 | Library | 153 | 0.00 | 0.00 | 0.00 | 0.00 | 145.45 | 168.05 | 0.00 | 0.00 | 100.00 |
| t808076 | Library | 154 | 22840.65 | 7649.22 | 1062.37 | 368.00 | 53.90 | 67.25 | 95.34 | 4.43 | 0.22 |
| t808093 | Library | 155 | 25568.84 | 4250.97 | 1228.66 | 49.24 | 25.19 | 50.38 | 95.33 | 4.58 | 0.09 |
| t808094 | Library | 156 | 4205.58 | 1662.08 | 42.93 | 85.87 | 0.00 | 0.00 | 98.99 | 1.01 | 0.00 |
| t808103 | Library | 157 | 19799.77 | 2081.79 | 1431.11 | 215.69 | 0.00 | 0.00 | 93.26 | 6.74 | 0.00 |
| t808125 | Library | 158 | 5001.66 | 1039.30 | 0.00 | 0.00 | 0.00 | 0.00 | 100.00 | 0.00 | 0.00 |
| t808154 | Library | 159 | 27499.73 | 2596.60 | 1409.40 | 108.39 | 474.23 | 30.33 | 93.59 | 4.80 | 1.61 |
| t808155 | Library | 160 | 8607.79 | 1672.46 | 173.09 | 202.50 | 0.00 | 0.00 | 98.03 | 1.97 | 0.00 |
| t808175 | Library | 161 | 12706.15 | 5621.21 | 457.36 | 89.70 | 0.00 | 0.00 | 96.53 | 3.47 | 0.00 |
| t808177 | Library | 162 | 29841.57 | 1319.33 | 1379.63 | 80.89 | 29.37 | 58.75 | 95.49 | 4.41 | 0.09 |
| t808199 | Library | 163 | 30105.67 | 6581.63 | 1428.21 | 352.46 | 361.60 | 265.65 | 94.39 | 4.48 | 1.13 |
| t808200 | Library | 164 | 29722.64 | 7533.35 | 1371.62 | 266.68 | 0.00 | 0.00 | 95.59 | 4.41 | 0.00 |
| t808223 | Library | 165 | 30389.40 | 2626.05 | 1438.41 | 75.78 | 191.90 | 45.95 | 94.91 | 4.49 | 0.60 |
| t808225 | Library | 166 | 27768.87 | 2462.17 | 1275.48 | 125.71 | 159.20 | 184.57 | 95.09 | 4.37 | 0.55 |
| t808226 | Library | 167 | 28398.51 | 6813.43 | 1301.73 | 240.36 | 306.20 | 87.33 | 94.64 | 4.34 | 1.02 |
| t808232 | Library | 168 | 20281.01 | 3554.46 | 1367.99 | 178.39 | 64.49 | 128.99 | 93.40 | 6.30 | 0.30 |
| t808237 | Library | 169 | 12281.96 | 2071.81 | 760.03 | 99.13 | 37.34 | 43.78 | 93.90 | 5.81 | 0.29 |
| t808238 | Library | 170 | 2934.86 | 2769.58 | 0.00 | 0.00 | 0.00 | 0.00 | 100.00 | 0.00 | 0.00 |
| t808240 | Library | 171 | 6248.43 | 606.29 | 115.70 | 141.31 | 0.00 | 0.00 | 98.18 | 1.82 | 0.00 |
| t808247 | Library | 172 | 27052.63 | 3600.04 | 1703.93 | 212.83 | 420.85 | 92.10 | 92.72 | 5.84 | 1.44 |
| t808253 | Library | 173 | 15518.14 | 8165.19 | 916.93 | 522.30 | 63.99 | 127.98 | 94.05 | 5.56 | 0.39 |

*The TS SEQ ID NOs provided in the table correspond to the complete protein sequence of each TS. In the context of the screen, two signal peptides were attached to each TS sequence. At the N-terminus, the N-terminal methionine was removed from each TS sequence, the TS sequence was linked to a signal peptide corresponding to SEQ ID NO: 16, and a methionine residue was added at the N-terminus of SEQ ID NO: 16. At the C-terminus, each TS sequence was linked to a signal peptide corresponding to SEQ ID NO: 17.

TABLE 11
CBCVA, THCVA, and CBDVA titers from metagenomic screening of CBCAS candidate enzymes in S. cerevisiae

| | | | Mean | Std Dev. | Mean | Std Dev. | Mean | Std Dev | | | |
|---|---|---|---|---|---|---|---|---|---|---|---|
| Strain | Strain | TS SEQ | CBCVA | CBCVA | THCVA | THCVA | CBDVA | CBDVA | % | % | % |
| ID | Type | ID NO* | [μg/L] | [μg/L] | [μg/L] | [μg/L] | [μg/L] | [μg/L] | CBCVA | THCVA | CBDVA |
| t807925 | A. niger CBCAS | 27 | 4473.59 | 1643.45 | 1821.60 | 462.56 | 13.83 | 30.48 | 70.91 | 28.87 | 0.22 |
| | Positive Control | | | | | | | | | | |
| t616313 | GFP | — | 319.32 | 903.18 | 230.36 | 651.57 | 0.00 | 0.00 | 58.09 | 41.91 | 0.00 |
| | Negative Control | | | | | | | | | | |
| t616314 | CBDAS | — | 19.93 | 56.36 | 44.29 | 48.47 | 1372.37 | 356.10 | 1.39 | 3.08 | 95.53 |
| | Positive Control | | | | | | | | | | |
| t701870 | THCAS | — | 280.12 | 32.10 | 9075.03 | 1061.25 | 0.00 | 0.00 | 2.99 | 97.01 | 0.00 |
| | Positive control | | | | | | | | | | |
| t807205 | Library | 104 | 3242.11 | 1268.65 | 1239.06 | 1024.00 | 12.91 | 25.81 | 72.14 | 27.57 | 0.29 |
| t807272 | Library | 105 | 4874.28 | 1877.01 | 1842.27 | 625.63 | 31.94 | 37.06 | 72.23 | 27.30 | 0.47 |
| t807301 | Library | 106 | 3187.10 | 614.37 | 1281.65 | 355.07 | 0.00 | 0.00 | 71.32 | 28.68 | 0.00 |
| t807677 | Library | 107 | 486.77 | 1114.57 | 3478.94 | 3901.07 | 0.00 | 0.00 | 12.27 | 87.73 | 0.00 |
| t807764 | Library | 108 | 4282.10 | 2666.67 | 1667.68 | 520.12 | 33.43 | 47.28 | 71.57 | 27.87 | 0.56 |
| t807774 | Library | 109 | 2245.66 | 252.04 | 1637.38 | 209.36 | 0.00 | 0.00 | 57.83 | 42.17 | 0.00 |
| t807810 | Library | 110 | 860.41 | 278.31 | 234.33 | 89.52 | 0.00 | 0.00 | 78.59 | 21.41 | 0.00 |
| t807822 | Library | 111 | 1114.76 | 1317.16 | 678.58 | 795.21 | 0.00 | 0.00 | 62.16 | 37.84 | 0.00 |
| t807854 | Library | 112 | 3821.61 | 376.51 | 820.39 | 99.73 | 0.00 | 0.00 | 82.33 | 17.67 | 0.00 |
| t807859 | Library | 113 | 0.00 | 0.00 | 0.00 | 0.00 | 0.00 | 0.00 | 0.00 | 0.00 | 0.00 |
| t807860 | Library | 114 | 1489.71 | 2036.35 | 925.30 | 1268.97 | 15.49 | 37.94 | 61.29 | 38.07 | 0.64 |
| t807861 | Library | 115 | 592.76 | 701.24 | 322.17 | 406.61 | 0.00 | 0.00 | 64.79 | 35.21 | 0.00 |
| t807863 | Library | 116 | 979.57 | 1212.94 | 366.25 | 470.73 | 0.00 | 0.00 | 72.79 | 27.21 | 0.00 |
| t807866 | Library | 117 | 947.47 | 1473.69 | 541.36 | 838.68 | 0.00 | 0.00 | 63.64 | 36.36 | 0.00 |
| t807869 | Library | 118 | 1969.26 | 1731.74 | 1700.80 | 1849.65 | 12.71 | 23.54 | 53.47 | 46.18 | 0.35 |
| t807873 | Library | 120 | 2573.41 | 469.37 | 1852.74 | 248.66 | 11.40 | 27.92 | 57.99 | 41.75 | 0.26 |
| t807878 | Library | 121 | 1509.38 | 1309.86 | 1003.24 | 903.57 | 7.68 | 21.71 | 59.89 | 39.81 | 0.30 |
| t807881 | Library | 122 | 1683.77 | 1656.75 | 754.52 | 884.88 | 7.88 | 22.28 | 68.83 | 30.84 | 0.32 |
| t807883 | Library | 123 | 1858.75 | 3607.91 | 687.66 | 1246.57 | 17.57 | 43.04 | 72.49 | 26.82 | 0.69 |
| t807917 | Library | 124 | 1836.22 | 655.01 | 703.90 | 291.09 | 0.00 | 0.00 | 72.29 | 27.71 | 0.00 |
| t807918 | Library | 125 | 2162.77 | 205.77 | 1837.88 | 182.53 | 27.72 | 32.03 | 53.69 | 45.62 | 0.69 |
| t807926 | Library | 126 | 2784.98 | 913.27 | 1285.08 | 336.14 | 0.00 | 0.00 | 68.43 | 31.57 | 0.00 |
| t807928 | Library | 127 | 2566.28 | 344.04 | 1132.43 | 91.93 | 0.00 | 0.00 | 69.38 | 30.62 | 0.00 |
| t807929 | Library | 128 | 2333.33 | 581.53 | 299.01 | 71.94 | 0.00 | 0.00 | 88.64 | 11.36 | 0.00 |
| t807930 | Library | 129 | 2442.49 | 556.63 | 1212.04 | 246.82 | 0.00 | 0.00 | 66.83 | 33.17 | 0.00 |
| t807933 | Library | 130 | 2408.56 | 692.63 | 1248.45 | 316.40 | 0.00 | 0.00 | 65.86 | 34.14 | 0.00 |
| t807943 | Library | 131 | 1986.17 | 677.42 | 756.16 | 148.38 | 0.00 | 0.00 | 72.43 | 27.57 | 0.00 |
| t807945 | Library | 132 | 161.04 | 188.87 | 0.00 | 0.00 | 0.00 | 0.00 | 100.00 | 0.00 | 0.00 |
| t807950 | Library | 134 | 3453.68 | 613.98 | 1656.86 | 240.06 | 0.00 | 0.00 | 67.58 | 32.42 | 0.00 |
| t807955 | Library | 135 | 1978.92 | 414.31 | 1415.78 | 302.91 | 0.00 | 0.00 | 58.29 | 41.71 | 0.00 |
| t807965 | Library | 136 | 2452.35 | 535.40 | 1538.67 | 349.32 | 0.00 | 0.00 | 61.45 | 38.55 | 0.00 |
| t807974 | Library | 137 | 165.89 | 331.78 | 29.12 | 58.23 | 0.00 | 0.00 | 85.07 | 14.93 | 0.00 |
| t807980 | Library | 138 | 3355.90 | 1222.41 | 554.16 | 669.40 | 35.61 | 45.18 | 85.05 | 14.04 | 0.90 |
| t808013 | Library | 139 | 1907.14 | 594.72 | 789.25 | 209.72 | 0.00 | 0.00 | 70.73 | 29.27 | 0.00 |
| t808014 | Library | 140 | 1762.19 | 360.60 | 1617.54 | 288.58 | 0.00 | 0.00 | 52.14 | 47.86 | 0.00 |
| t808021 | Library | 141 | 4204.55 | 218.50 | 1774.95 | 79.34 | 0.00 | 0.00 | 70.32 | 29.68 | 0.00 |
| t808022 | Library | 142 | 4422.08 | 738.43 | 1809.05 | 196.16 | 0.00 | 0.00 | 70.97 | 29.03 | 0.00 |
| t808024 | Library | 143 | 2908.72 | 808.06 | 1276.65 | 416.04 | 20.03 | 40.05 | 69.17 | 30.36 | 0.48 |
| t808026 | Library | 144 | 3270.32 | 422.75 | 1713.13 | 176.34 | 18.76 | 37.52 | 65.38 | 34.25 | 0.38 |
| t808029 | Library | 145 | 2406.20 | 1183.56 | 953.65 | 712.38 | 18.18 | 36.36 | 71.23 | 28.23 | 0.54 |
| t808039 | Library | 146 | 2104.54 | 404.51 | 747.24 | 150.97 | 0.00 | 0.00 | 73.80 | 26.20 | 0.00 |
| t808040 | Library | 147 | 2925.68 | 1239.54 | 938.63 | 809.00 | 14.93 | 29.87 | 75.42 | 24.20 | 0.38 |
| t808041 | Library | 148 | 152.65 | 111.77 | 0.00 | 0.00 | 0.00 | 0.00 | 100.00 | 0.00 | 0.00 |
| t808045 | Library | 149 | 0.00 | 0.00 | 9402.99 | 1132.41 | 0.00 | 0.00 | 0.00 | 100.00 | 0.00 |
| t808046 | Library | 150 | 3174.69 | 772.59 | 1514.30 | 295.18 | 0.00 | 0.00 | 67.71 | 32.29 | 0.00 |
| t808051 | Library | 151 | 2863.45 | 1434.93 | 2043.57 | 770.01 | 33.01 | 38.44 | 57.96 | 41.37 | 0.67 |
| t808061 | Library | 152 | 2367.22 | 114.94 | 1495.44 | 77.78 | 0.00 | 0.00 | 61.28 | 38.72 | 0.00 |
| t808069 | Library | 153 | 0.00 | 0.00 | 0.00 | 0.00 | 169.41 | 210.59 | 0.00 | 0.00 | 100.00 |
| t808076 | Library | 154 | 3558.84 | 124.32 | 1458.01 | 189.04 | 0.00 | 0.00 | 70.94 | 29.06 | 0.00 |
| t808093 | Library | 155 | 3833.15 | 875.00 | 1280.76 | 906.89 | 35.24 | 41.80 | 74.44 | 24.87 | 0.68 |
| t808094 | Library | 156 | 2498.54 | 925.99 | 808.41 | 353.22 | 0.00 | 0.00 | 75.55 | 24.45 | 0.00 |
| t808103 | Library | 157 | 2911.66 | 912.45 | 2038.06 | 496.29 | 25.07 | 50.15 | 58.53 | 40.97 | 0.50 |
| t808125 | Library | 158 | 3288.83 | 840.09 | 595.19 | 150.14 | 0.00 | 0.00 | 84.68 | 15.32 | 0.00 |
| t808154 | Library | 159 | 3740.08 | 532.10 | 1882.39 | 217.34 | 0.00 | 0.00 | 66.52 | 33.48 | 0.00 |
| t808155 | Library | 160 | 4173.38 | 1767.24 | 1063.02 | 315.81 | 0.00 | 0.00 | 79.70 | 20.30 | 0.00 |
| t808175 | Library | 161 | 1838.07 | 137.92 | 635.41 | 516.48 | 8.73 | 17.47 | 74.05 | 25.60 | 0.35 |
| t808177 | Library | 162 | 3018.88 | 539.22 | 1053.71 | 728.24 | 17.94 | 35.88 | 73.80 | 25.76 | 0.44 |
| t808199 | Library | 163 | 3733.69 | 2406.71 | 1651.60 | 1693.22 | 25.91 | 51.83 | 69.00 | 30.52 | 0.48 |
| t808200 | Library | 164 | 3073.87 | 538.39 | 1507.03 | 239.98 | 0.00 | 0.00 | 67.10 | 32.90 | 0.00 |
| t808223 | Library | 165 | 3592.30 | 439.40 | 1636.00 | 155.56 | 0.00 | 0.00 | 68.71 | 31.29 | 0.00 |
| t808225 | Library | 166 | 3608.44 | 825.78 | 1476.48 | 1038.39 | 27.78 | 55.57 | 70.58 | 28.88 | 0.54 |
| t808226 | Library | 167 | 4553.40 | 2121.13 | 2421.15 | 654.66 | 64.86 | 48.17 | 64.68 | 34.39 | 0.92 |
| t808232 | Library | 168 | 2379.23 | 352.45 | 1626.74 | 243.69 | 0.00 | 0.00 | 59.39 | 40.61 | 0.00 |
| t808237 | Library | 169 | 3599.07 | 1154.82 | 1273.10 | 423.30 | 0.00 | 0.00 | 73.87 | 26.13 | 0.00 |
| t808238 | Library | 170 | 1841.90 | 684.06 | 282.10 | 414.39 | 0.00 | 0.00 | 86.72 | 13.28 | 0.00 |
| t808240 | Library | 171 | 4282.13 | 1030.26 | 888.75 | 394.89 | 0.00 | 0.00 | 82.81 | 17.19 | 0.00 |
| t808247 | Library | 172 | 2651.55 | 513.12 | 1783.19 | 177.55 | 15.16 | 30.31 | 59.59 | 40.07 | 0.34 |
| t808253 | Library | 173 | 1476.99 | 735.60 | 715.85 | 720.66 | 0.00 | 0.00 | 67.35 | 32.65 | 0.00 |

*The TS SEQ ID NOs provided in the table correspond to the complete protein sequence of each TS. In the context of the screen, two signal peptides were attached to each TS sequence. At the N-terminus, the N-terminal methionine was removed from each TS sequence, the TS sequence was linked to a signal peptide corresponding to SEQ ID NO: 16, and a methionine residue was added at the N-terminus of SEQ ID NO: 16. At the C-terminus, each TS sequence was linked to a signal peptide corresponding to SEQ ID NO: 17.

TABLE 12
CBGA and CBGVA residual substrate from metagenomic screening
of CBCAS candidate enzymes in S. cerevisiae

| | | | | Standard | | Standard |
|---|---|---|---|---|---|---|
| | | | Average | Deviation | Average | Deviation |
| Strain | | TS SEQ | CBGA | CBGA | CBGVA | CBGVA |
| ID | Strain Type | ID NO* | [μg/L] | [μg/L] | [μg/L] | [μg/L] |
| t807925 | A. niger CBCAS | 27 | 19.90 | 45.80 | 0.00 | 0.00 |
| | Positive Control | | | | | |
| t616313 | GFP | — | 59298.53 | 5174.35 | 21898.05 | 10583.34 |
| | Negative Control | | | | | |
| t807205 | Library | 104 | 53147.96 | 12834.43 | 3437.64 | 2892.55 |
| t807272 | Library | 105 | 0.00 | 0.00 | 0.00 | 0.00 |
| t807301 | Library | 106 | 0.00 | 0.00 | 0.00 | 0.00 |
| t807677 | Library | 107 | 52271.45 | 7668.39 | 11977.90 | 8565.71 |
| t807764 | Library | 108 | 40451.56 | 9639.86 | 311.78 | 236.61 |
| t807774 | Library | 109 | 32.82 | 65.65 | 0.00 | 0.00 |
| t807810 | Library | 110 | 380.38 | 703.07 | 0.00 | 0.00 |
| t807822 | Library | 111 | 538.72 | 1077.45 | 16.99 | 33.97 |
| t807854 | Library | 112 | 8963.64 | 3478.68 | 0.00 | 0.00 |
| t807859 | Library | 113 | 63345.00 | 14967.80 | 17522.15 | 3427.61 |
| t807860 | Library | 114 | 43908.19 | 31951.06 | 9772.66 | 11054.13 |
| t807861 | Library | 115 | 62687.37 | 12260.30 | 16647.73 | 4876.37 |
| t807863 | Library | 116 | 48851.59 | 9711.58 | 16336.42 | 8135.29 |
| t807866 | Library | 117 | 36035.77 | 11249.90 | 10751.97 | 9127.95 |
| t807869 | Library | 118 | 42005.98 | 26148.08 | 7246.67 | 9148.58 |
| t807873 | Library | 120 | 20.28 | 49.68 | 0.00 | 0.00 |
| t807878 | Library | 121 | 38442.99 | 20155.33 | 5151.50 | 7882.93 |
| t807881 | Library | 122 | 46732.64 | 18976.53 | 11406.58 | 10063.07 |
| t807883 | Library | 123 | 42814.16 | 9130.34 | 12651.49 | 10100.78 |
| t807917 | Library | 124 | 0.00 | 0.00 | 0.00 | 0.00 |
| t807918 | Library | 125 | 0.00 | 0.00 | 0.00 | 0.00 |
| t807926 | Library | 126 | 57.58 | 67.71 | 0.00 | 0.00 |
| t807928 | Library | 127 | 25.47 | 50.94 | 0.00 | 0.00 |
| t807929 | Library | 128 | 41396.36 | 27087.65 | 15214.71 | 1846.68 |
| t807930 | Library | 129 | 44.74 | 89.48 | 0.00 | 0.00 |
| t807933 | Library | 130 | 0.00 | 0.00 | 0.00 | 0.00 |
| t807943 | Library | 131 | 0.00 | 0.00 | 0.00 | 0.00 |
| t807945 | Library | 132 | 55188.82 | 15675.50 | 22716.84 | 10015.46 |
| t807950 | Library | 134 | 0.00 | 0.00 | 0.00 | 0.00 |
| t807955 | Library | 135 | 0.00 | 0.00 | 0.00 | 0.00 |
| t807965 | Library | 136 | 0.00 | 0.00 | 0.00 | 0.00 |
| t807974 | Library | 137 | 48233.77 | 33615.86 | 20337.45 | 1273.42 |
| t807980 | Library | 138 | 0.00 | 0.00 | 0.00 | 0.00 |
| t808013 | Library | 139 | 35.97 | 71.94 | 0.00 | 0.00 |
| t808014 | Library | 140 | 0.00 | 0.00 | 0.00 | 0.00 |
| t808021 | Library | 141 | 0.00 | 0.00 | 0.00 | 0.00 |
| t808022 | Library | 142 | 0.00 | 0.00 | 0.00 | 0.00 |
| t808024 | Library | 143 | 0.00 | 0.00 | 0.00 | 0.00 |
| t808026 | Library | 144 | 0.00 | 0.00 | 0.00 | 0.00 |
| t808029 | Library | 145 | 39.53 | 79.06 | 0.00 | 0.00 |
| t808039 | Library | 146 | 53.06 | 106.12 | 0.00 | 0.00 |
| t808040 | Library | 147 | 10397.55 | 7554.81 | 60.04 | 72.45 |
| t808041 | Library | 148 | 43557.01 | 9983.47 | 30246.69 | 9758.25 |
| t808045 | Library | 149 | 575.78 | 450.99 | 0.00 | 0.00 |
| t808046 | Library | 150 | 0.00 | 0.00 | 0.00 | 0.00 |
| t808051 | Library | 151 | 28.31 | 56.61 | 0.00 | 0.00 |
| t808061 | Library | 152 | 34.71 | 69.42 | 0.00 | 0.00 |
| t808069 | Library | 153 | 53474.30 | 8943.22 | 13875.42 | 911.61 |
| t808076 | Library | 154 | 0.00 | 0.00 | 0.00 | 0.00 |
| t808093 | Library | 155 | 0.00 | 0.00 | 0.00 | 0.00 |
| t808094 | Library | 156 | 31781.07 | 13527.80 | 2741.81 | 2696.82 |
| t808103 | Library | 157 | 0.00 | 0.00 | 0.00 | 0.00 |
| t808125 | Library | 158 | 53834.41 | 9317.13 | 3639.01 | 1236.20 |
| t808154 | Library | 159 | 1056.05 | 420.68 | 0.00 | 0.00 |
| t808155 | Library | 160 | 21117.02 | 9763.61 | 23.86 | 47.72 |
| t808175 | Library | 161 | 8034.51 | 16069.03 | 0.00 | 0.00 |
| t808177 | Library | 162 | 0.00 | 0.00 | 0.00 | 0.00 |
| t808199 | Library | 163 | 0.00 | 0.00 | 0.00 | 0.00 |
| t808200 | Library | 164 | 0.00 | 0.00 | 0.00 | 0.00 |
| t808223 | Library | 165 | 0.00 | 0.00 | 0.00 | 0.00 |
| t808225 | Library | 166 | 0.00 | 0.00 | 0.00 | 0.00 |
| t808226 | Library | 167 | 0.00 | 0.00 | 0.00 | 0.00 |
| t808232 | Library | 168 | 0.00 | 0.00 | 0.00 | 0.00 |
| t808237 | Library | 169 | 69.20 | 138.40 | 0.00 | 0.00 |
| t808238 | Library | 170 | 63815.30 | 9562.86 | 9247.47 | 6162.29 |
| t808240 | Library | 171 | 24393.82 | 2396.56 | 4054.85 | 4444.75 |
| t808247 | Library | 172 | 0.00 | 0.00 | 0.00 | 0.00 |
| t808253 | Library | 173 | 0.00 | 0.00 | 0.00 | 0.00 |

*The TS SEQ ID NOs provided in the table correspond to the complete protein sequence of each TS. In the context of the screen, two signal peptides were attached to each TS sequence. At the N-terminus, the N-terminal methionine was removed from each TS sequence, the TS sequence was linked to a signal peptide corresponding to SEQ ID NO: 16, and a methionine residue was added at the N-terminus of SEQ ID NO: 16. At the C-terminus, each TS sequence was linked to a signal peptide corresponding to SEQ ID NO: 17.

### Example 4: Assessment of the Requirement for Signal Peptides for CBCAS Activity

Post-translational modifications (e.g., the formation of intramolecular disulfide bridges, post-translational glycosylation, etc.) are known to be important for the activity of Cannabis terminal synthases. The presence of signal peptides on terminal synthase enzymes may help facilitate the post-translational modifications. However, it was unknown whether the A. niger CBCAS identified in Example 1, or the additional CBCASs identified in Example 3, required signal peptides to be active.

A library of 20 CBCAS enzymes selected from Example 1 and 3 was synthesized, including versions of the CBCAS enzymes with and without the N-terminal MFalpha2 signal peptide (SEQ ID NO: 16) and C-terminal HDEL signal peptide (SEQ ID NO: 17). Each candidate enzyme expression construct was transformed into an S. cerevisiae CEN.PK strain that also expressed a prenyltransferase enzyme capable of catalyzing reaction R⁴ in FIG. 2. Strain t861555 expressing the A. niger CBCAS identified in Example 1, carrying both the Mfalpha2 and HDEL signal peptides was included in the library screen as a positive control for enzyme activity. Strain t861565 expressed the same A. niger CBCAS without the Mfalpha2 and HDEL signal peptides.

The strains were screened using the assay described in Example 1 with the following exception: at Day 4 samples were not subjected to a pH adjustment and a further 2 days of incubation at 20° C.

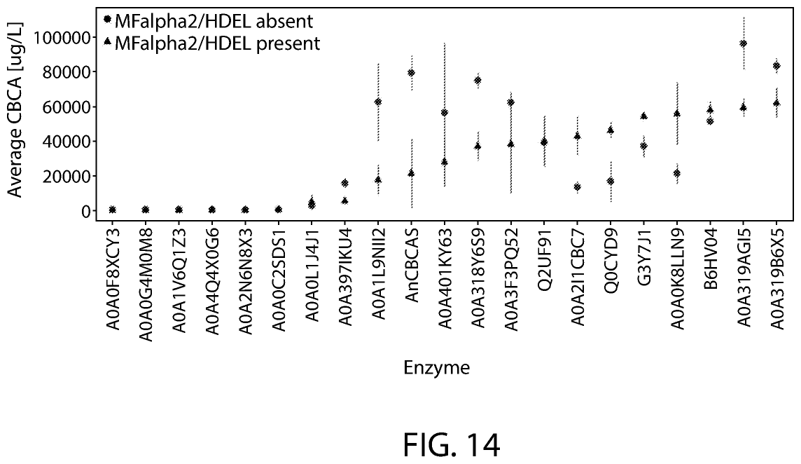

12 strains demonstrated greater mean CBCAS activity than that of the t861555 positive control (FIG. 14, Table 13). Surprisingly, the impact of the two signal peptides was found to vary depending on the identity of the CBCAS candidate: in some instances, the presence of both signal peptides was observed to enhance CBCAS activity, while in other instances, it was observed to reduce activity. The absence of the two signal peptides from the A. niger CBCAS had a significant positive impact on CBCAS activity. The t861565 strain, expressing the A. niger CBCAS without signal peptides demonstrated approximately 4-fold higher CBCA titer than the t861555 strain, expressing the A. niger CBCAS with signal peptides.

TABLE 13
CBCA titers from screening of CBCAS candidate enzymes
with and without signal peptides in S. cerevisiae

| | | | N-terminal and | | |
|---|---|---|---|---|---|
| | | | C-terminal | | Standard |
| | | | peptides | Average | Deviation |
| Strain | | TS SEQ | [Y = Yes | CBCA | CBCA |
| ID | Strain Type | ID NO* | N = No] | [μg/L] | [μg/L] |
| t861555 | A. niger CBCAS | 27 | Y | 21237.64 | 22960.70 |
| | Pos. Ctrl. | | | | |
| t861565 | A. niger CBCAS | 27 | N | 78892.80 | 10755.89 |
| | Pos. Ctrl. | | | | |
| t861557 | Library | 144 | Y | 520.64 | 901.77 |
| t861584 | Library | 144 | N | 0.00 | 0.00 |
| t861559 | Library | 150 | Y | 0.00 | 0.00 |
| t861586 | Library | 150 | N | 0.00 | 0.00 |
| t861591 | Library | 141 | Y | 0.00 | 0.00 |
| t861573 | Library | 141 | N | 0.00 | 0.00 |
| t861562 | Library | 167 | Y | 55737.91 | 20610.57 |
| t861582 | Library | 167 | N | 20912.35 | 6804.79 |
| t861563 | Library | 112 | Y | 4821.60 | 3851.63 |
| t861553 | Library | 112 | N | 2393.08 | 2024.49 |
| t861551 | Library | 105 | Y | 17501.94 | 8781.47 |
| t861578 | Library | 105 | N | 62171.35 | 31734.93 |
| t861568 | Library | 142 | Y | 0.00 | 0.00 |
| t861576 | Library | 142 | N | 0.00 | 0.00 |
| t861588 | Library | 163 | Y | 42686.95 | 11722.91 |
| t861564 | Library | 163 | N | 12924.20 | 3312.59 |
| t861567 | Library | 154 | Y | 0.00 | 0.00 |
| t861575 | Library | 154 | N | 0.00 | 0.00 |
| t861577 | Library | 126 | Y | 36869.19 | 8966.99 |
| t861592 | Library | 126 | N | 74584.36 | 5016.15 |
| t861583 | Library | 162 | Y | 59260.52 | 5672.49 |
| t861589 | Library | 162 | N | 95796.21 | 18887.68 |
| t861566 | Library | 155 | Y | 61918.09 | 9713.74 |
| t861587 | Library | 155 | N | 82883.01 | 5160.26 |
| t861554 | Library | 159 | Y | 5334.71 | N/A** |
| t861552 | Library | 159 | N | 15253.62 | 3086.10 |
| t861574 | Library | 164 | Y | 38142.03 | 31232.36 |
| t861572 | Library | 164 | N | 61793.56 | 7141.71 |
| t861558 | Library | 134 | Y | 27898.00 | 15692.88 |
| t861590 | Library | 134 | N | 55852.93 | 43778.21 |
| t861580 | Library | 143 | Y | 0.00 | 0.00 |
| t861570 | Library | 143 | N | 0.00 | 0.00 |
| t861579 | Library | 172 | Y | 57912.84 | 5105.04 |
| t861556 | Library | 172 | N | 50870.36 | 1457.77 |
| t861571 | Library | 165 | Y | 54271.76 | 2447.30 |
| t861569 | Library | 165 | N | 36631.83 | 6800.49 |
| t861561 | Library | 166 | Y | 46161.25 | 5238.08 |
| t861560 | Library | 166 | N | 16325.34 | 14173.22 |
| t861585 | Library | 130 | Y | 39673.45 | 15792.21 |
| t861581 | Library | 130 | N | 38663.23 | 6553.85 |

*The TS SEQ ID NOs provided in the table correspond to the complete protein sequence of each TS. In the context of the screen, for the strains that are indicated as “Y” for expressing the TS sequence with signal peptides, two signal peptides were attached to each TS sequence. At the N-terminus, the N-terminal methionine was removed from each TS sequence, the TS sequence was linked to a signal peptide corresponding to SEQ ID NO: 16, and a methionine residue was added at the N-terminus of SEQ ID NO: 16. At the C-terminus, each TS sequence was linked to a signal peptide corresponding to SEQ ID NO: 17.
**single bioreplicate, standard deviation not applicable

### Example 5: Identification of Sequence Motifs Enriched in CBCAS Enzymes Identified in Examples 1-4

Analysis of CBCAS enzymes from Example 4 identified multiple sequence motifs that were enriched in CBCAS enzymes that produced a mean CBCA titer greater than the A. niger CBCAS. Table 14 provides sequence information for the motifs identified.

Structural models were generated using crystal structures from related proteins to determine where the sequence motifs localize within the 3-dimensional structure of a TS enzyme. FIGS. 15 and 16 depict ribbon diagrams showing predicted localization of several of the identified sequence motifs. Sequence motifs KVQARSGGH (SEQ ID NO: 174), CPTI[KR]TGGH (SEQ ID NO: 181), and P[IV]S[DQE]TTY[EDG]F[TA]DGLYDVLA[RQK]AVPES[VA]GHAYLGCPDP[RK]M (SEQ ID NO: 186), indicated by arrows in FIG. 15, are predicted to contact the cofactor binding site and may therefore influence cofactor binding.

The motif RT[EQ][PQ]APGLAVQYSY (SEQ ID NO: 207), indicated by an arrow in FIG. 16, is predicted to be near the substrate binding pocket. The motif WQ[SA]FI[SA][AQ][KE]NLT[RW][QK]FY[NST]NM (SEQ ID NO: 211), indicated by an arrow in FIG. 16, is predicted to line the cavity of the active site and may potentially influence substrate or product specificity.

TABLE 14
Motif sequences identified in candidate CBCASs

| | Reference | | | | |
|---|---|---|---|---|---|
| | sequence (SEQ | | | | |
| | ID NO: 27) | | Motif sequence | | TS SEQ |
| Motif | start | end | in strain | Strain* | ID NO** |
| KVQARSGGH (SEQ ID NO: 174) | 72 | 80 | KVQARSGGH | t861555 | 27 |
| | | | (SEQ ID NO: | t861565 | |
| | | | 174) | t861579 | 172 |
| | | | | t861556 | |
| | | | | t861561 | 166 |
| | | | | t861560 | |
| | | | | t861554 | 159 |
| | | | | t861552 | |
| | | | | t861588 | 163 |
| | | | | t861564 | |
| | | | | t861562 | 167 |
| | | | | t861582 | |
| | | | | t861571 | 165 |
| | | | | t861569 | |
| | | | | t861583 | 162 |
| | | | | t861589 | |
| | | | | t861558 | 134 |
| | | | | t861590 | |
| | | | | t861574 | 164 |
| | | | | t861572 | |
| | | | | t861551 | 105 |
| | | | | t861578 | |
| | | | | t861577 | 126 |
| | | | | t861592 | |
| | | | | t861566 | 155 |
| | | | | t861587 | |
| | | | | t861563 | 112 |
| | | | | t861553 | |
| | | | | t861585 | 130 |
| | | | | t861581 | |
| RASNTQNQD[VI][FL]FA[VI]K (SEQ | 183 | 197 | RASNTQNQDVF | t861555 | 27 |
| ID NO: 176) | | | FAVK (SEQ ID | t861565 | |
| | | | NO: 177) | t861571 | 165 |
| | | | | t861569 | |
| | | | | t861583 | 162 |
| | | | | t861589 | |
| | | | | t861558 | 134 |
| | | | | t861590 | |
| | | | | t861574 | 164 |
| | | | | t861572 | |
| | | | | t861551 | 105 |
| | | | | t861578 | |
| | | | | t861566 | 155 |
| | | | | t861587 | |
| | | | RASNTQNQDIL | t861579 | 172 |
| | | | FAVK (SEQ ID | t861556 | |
| | | | NO: 178) | | |
| | | | RASNTQNQDIL | t861588 | 163 |
| | | | FAIK (SEQ ID | t861564 | |
| | | | NO: 179) | | |
| | | | RASNTQNQDV | t861577 | 126 |
| | | | LFAVK (SEQ ID | t861592 | |
| | | | NO: 180) | | |
| CPTI[KR]TGGH (SEQ ID NO: 181) | 141 | 149 | CPTIKTGGH | t861555 | 27 |
| | | | (SEQ ID NO: | t861565 | |
| | | | 182) | t861571 | 165 |
| | | | | t861569 | |
| | | | | t861583 | 162 |
| | | | | t861589 | |
| | | | | t861558 | 134 |
| | | | | t861590 | |
| | | | | t861574 | 164 |
| | | | | t861572 | |
| | | | | t861551 | 105 |
| | | | | t861578 | |
| | | | | t861577 | 126 |
| | | | | t861592 | |
| | | | | t861566 | 155 |
| | | | | t861587 | |
| | | | CPTIRTGGH | t861579 | 172 |
| | | | (SEQ ID NO: | t861556 | |
| | | | 183) | t861561 | 166 |
| | | | | t861560 | |
| | | | | t861554 | 159 |
| | | | | t861552 | |
| | | | | t861588 | 163 |
| | | | | t861564 | |
| | | | | t861562 | 167 |
| | | | | t861582 | |
| WFVTLSLEGGAINDV[AP]EDATAY | 360 | 383 | WFVTLSLEGGA | t861555 | 27 |
| [AG]H (SEQ ID NO: 184) | | | INDVAEDATAY | t861565 | |
| | | | AH (SEQ ID NO: | t861571 | 165 |
| | | | 185) | t861569 | |
| | | | | t861583 | 162 |
| | | | | t861589 | |
| | | | | t861551 | 105 |
| | | | | t861578 | |
| | | | | t861577 | 126 |
| | | | | t861592 | |
| | | | | t861566 | 155 |
| | | | | t861587 | |
| P[IV]S[DQE]TTY[EDG]F[TA]DGLY | 400 | 436 | PISDTTYEFTDG | t861555 | 27 |
| DVLA[RQK]AVPES[VA]GHAYLGC | | | SVGHAYLGCPD | t861565 | |
| PDP[RK]M (SEQ ID NO: 186) | | | LYDVLARAVPE | t861571 | 165 |
| | | | PRM (SEQ ID | t861569 | |
| | | | NO: 187) | t861583 | 162 |
| | | | | t861589 | |
| | | | | t861558 | 134 |
| | | | | t861590 | |
| | | | | t861574 | 164 |
| | | | | t861572 | |
| | | | PISETTYEFTDG | t861551 | 105 |
| | | | LYDVLARAVPE | t861578 | |
| | | | SVGHAYLGCPD | t861577 | 126 |
| | | | PRM (SEQ ID | t861592 | |
| | | | NO: 188) | t861566 | 155 |
| | | | | t861587 | |
| MKHF[TNS]QFSM (SEQ ID NO: 189) | 98 | 106 | MKHFTQFSM | t861555 | 27 |
| | | | (SEQ ID NO: | t861565 | |
| | | | 190) | t861571 | 165 |
| | | | | t861569 | |
| | | | | t861583 | 162 |
| | | | | t861589 | |
| | | | | t861558 | 134 |
| | | | | t861590 | |
| | | | | t861574 | 164 |
| | | | | t861572 | |
| | | | | t861563 | 112 |
| | | | | t861553 | |
| | | | MKHFSQFSM | t861579 | 172 |
| | | | (SEQ ID NO: | t861556 | |
| | | | 191) | t861561 | 166 |
| | | | | t861560 | |
| | | | | t861554 | 159 |
| | | | | t861552 | |
| | | | | t861562 | 167 |
| | | | | t861582 | |
| | | | MKHFNQFSM | t861588 | 163 |
| | | | (SEQ ID NO: | t861564 | |
| | | | 192) | t861551 | 105 |
| | | | | t861578 | |
| | | | | t861577 | 126 |
| | | | | t861592 | |
| | | | | t861566 | 155 |
| | | | | t861587 | |
| P[EQ][TS]A[EAD][QE]IA[GA][VI]V | 53 | 65 | PETAEQIAGIVK | t861574 | 164 |
| KC (SEQ ID NO: 193) | | | C (SEQ ID NO: | t861572 | |
| | | | 194) | t861551 | 105 |
| | | | | t861578 | |
| | | | | t861577 | 126 |
| | | | | t861592 | |
| | | | | t861566 | 155 |
| | | | | t861587 | |
| | | | PQSADEIAAVV | t861554 | 159 |
| | | | KC (SEQ ID NO: | t861552 | |
| | | | 195) | t861588 | 163 |
| | | | | t861564 | |
| | | | | t861562 | 167 |
| | | | | t861582 | |
| | | | PETAAQIAGVV | t861555 | 27 |
| | | | KC (SEQ ID NO: | t861565 | |
| | | | 196) | t861571 | 165 |
| | | | | t861569 | |
| | | | | t861583 | 162 |
| | | | | t861589 | |
| | | | PQSAEEIAAVV | t861579 | 172 |
| | | | KC (SEQ ID NO: | t861556 | |
| | | | 197) | | |
| | | | PETAEQIAGVV | t861558 | 134 |
| | | | KC (SEQ ID NO: | t861590 | |
| | | | 198) | | |
| | | | PETAEQIAAVV | t861585 | 130 |
| | | | KC (SEQ ID NO: | t861581 | |
| | | | 199) | | |
| RDCL[IV]SA[LV]GGN[SA]A[LH][AV] | 10 | 32 | RDCLISAVGGN | t861561 | 166 |
| [AV]F[PQ][ND][QE]LL[WY] (SEQ | | | AAHVAFQDQL | t861560 | |
| ID NO: 200) | | | LY (SEQ ID NO: | t861562 | 167 |
| | | | 201) | t861582 | |
| | | | RDCLISALGGN | t861555 | 27 |
| | | | SALAVFPNELL | t861565 | |
| | | | W (SEQ ID NO: | t861571 | 165 |
| | | | 202) | t861569 | |
| | | | | t861583 | 162 |
| | | | | t861589 | |
| | | | RDCLISALGGN | 1861558 | 134 |
| | | | SALAAFPNELL | t861590 | |
| | | | W (SEQ ID NO: | t861574 | 164 |
| | | | 203) | t861572 | |
| | | | RDCLISALGGN | t861551 | 105 |
| | | | SALAVFPNQLL | t861578 | |
| | | | W (SEQ ID NO: | | |
| | | | 204) | | |
| | | | RDCLISALGGN | t861577 | 126 |
| | | | SALAAFPNQLL | t861592 | |
| | | | W (SEQ ID NO: | | |
| | | | 205) | | |
| | | | RDCLVSALGGN | t861566 | 155 |
| | | | SALAAFPNQLL | t861587 | |
| | | | W (SEQ ID NO: | | |
| | | | 206) | | |
| RT[EQ][PQ]APGLAVQYSY (SEQ ID | 212 | 225 | RTEPAPGLAVQ | t861555 | 27 |
| NO: 207) | | | YSY (SEQ ID | t861565 | |
| | | | NO: 208) | t861571 | 165 |
| | | | | t861569 | |
| | | | | t861583 | 162 |
| | | | | t861589 | |
| | | | | t861558 | 134 |
| | | | | t861590 | |
| | | | | t861574 | 164 |
| | | | | t861572 | |
| | | | | t861551 | 105 |
| | | | | t861578 | |
| | | | | t861577 | 126 |
| | | | | t861592 | |
| | | | | t861566 | 155 |
| | | | | t861587 | |
| | | | RTEQAPGLAVQ | t861561 | 166 |
| | | | YSY (SEQ ID | t861560 | |
| | | | NO: 209) | t861562 | 167 |
| | | | | t861582 | |
| | | | RTQPAPGLAVQ | t861563 | 112 |
| | | | YSY (SEQ ID | t861553 | |
| | | | NO: 210) | | |
| WQ[SA]FI[SA][AQ][KE]NLT[RW] | 242 | 259 | WQSFISAKNLT | t861555 | 27 |
| [QK]FY[NST]NM (SEQ ID NO: 211) | | | RQFYNNM | t861565 | |
| | | | (SEQ ID NO: | t861571 | 165 |
| | | | 212) | t861569 | |
| | | | | t861583 | 162 |
| | | | | t861589 | |
| | | | | t861558 | 134 |
| | | | | t861590 | |
| | | | | t861574 | 164 |
| | | | | t861572 | |
| | | | | t861551 | 105 |
| | | | | t861578 | |
| | | | | t861577 | 126 |
| | | | | t861592 | |
| | | | | t861566 | 155 |
| | | | | t861587 | |
| | | | WQSFISAKNLT | t861563 | 112 |
| | | | RQFYTNM (SEQ | t861553 | |
| | | | ID NO: 213) | | |

*The table includes two strains for every TS, based on data presented in Example 4. For each TS, one strain expressed the TS with signal peptides (top row for each strain) and one strain expressed the TS without signal peptides (bottom row for each strain).
**The TS SEQ ID NOs provided in the table correspond to the complete protein sequence of each TS. In the context of the screen, for the strains that expressed the TS with signal peptides (top row for each strain), two signal peptides were attached to each TS sequence. At the N-terminus, the N-terminal methionine was removed from each TS sequence, the TS sequence was linked to a signal peptide corresponding to SEQ ID NO: 16, and a methionine residue was added at the N-terminus of SEQ ID NO: 16. At the C-terminus, each TS sequence was linked to a signal peptide corresponding to SEQ ID NO: 17.

### Example 6: Biosynthesis of Cannabinoids in Engineered S. cerevisiae Host Cells

The activation of an organic acid to its CoA-thioester and the subsequent condensation of this thioester with a number of malonyl-CoA molecules, or other similar polyketide extender units, represent the first two steps in the biosynthesis of all known cannabinoids. To demonstrate the biosynthesis of CBGA (FIG. 1, Formula 8a), CBDA (FIG. 1, Formula 9a), THCA (FIG. 1, Formula 10a), and/or CBCA (FIG. 1, Formula 11a) the cannabinoid biosynthetic pathway shown in FIG. 1 is assembled in the genome of a prototrophic S. cerevisiae CEN.PK host cell wherein each enzyme (R1a-R5a) may be present in one or more copies. For example, the S. cerevisiae host cell may express one or more copies of one or more of: an AAE, an OLS, an OAC, a PT, and a TS.

The AAE enzyme used may be a naturally occurring or synthetic AAE that is functionally expressed in S. cerevisiae, or a variant thereof, with activity on hexanaoic acid. The OLS enzyme may be a naturally occurring or synthetic OLS that is functionally expressed in S. cerevisiae. The OAC enzyme may be a naturally occurring or synthetic OAC that is functionally expressed in S. cerevisiae. In instances where a bifunctional OLS is used, a separate OAC enzyme may or may not be omitted. The PT enzyme may be a naturally occurring or synthetic PT that is functionally expressed in S. cerevisiae.

A TS enzyme may be a naturally occurring or synthetic TS that is functionally expressed in S. cerevisiae, or a variant thereof, including a TS from C. sativa, a variant of a TS from C. sativa, and/or a TS from a non-Cannabis species. The TS enzyme may be a TS that produces one or more of CBCA, CBCVA, THCA, THCVA, CBDA, and CBDVA as a majority product. The TS enzyme may comprise one or more of the TS enzymes provided in this disclosure.

The cannabinoid fermentation procedure may be similar to the assays described in the Examples above, except that the incubation of production cultures may last from, for example, 48-144 hours and production cultures may be supplemented with, for example, 4% galactose and 1 mM sodium hexanoate every 24 hours. Titers of CBCA, CBCVA, THCA, THCVA, CBDA, and CBDVA are quantified via LC-MS.

### Sequences Associated with the Disclosure

Table 15. Sequences of Candidate CBCASs Described in Example 3* and Example 4** *For the library screen in Example 3, the TS sequences provided in Table 15 were expressed with an N-terminal MFalpha2 signal peptide (SEQ ID NO: 16) and a C-terminal HDEL signal peptide (SEQ ID NO: 17). The methionine residue was removed from the N-terminus of the TS sequences provided in FIG. 15. A methionine residue was instead added at the N-terminus of SEQ ID NO: 16.**For the library screen in Example 4, the TS sequences were expressed with and without N-terminal and C-terminal signal peptides. For TS sequences expressed with signal peptides, the same approach as described above for Example 3 was used.

| | | | SEQ | | SEQ |
|---|---|---|---|---|---|
| Strain | Strain | | ID | | ID |
| ID | Type | Nucleotide Sequence | NO: | Amino Acid Sequence | NO: |
| t807925 | t807925 | atgggtaatacgacctctattgccggcagagattgtttg | 28 | MGNTTSIAGRDCLIS | 27 |
| | A. niger | atctcagctttaggtggtaactccgctcttgcagtttttcc | | ALGGNSALAVFPNE | |
| | CBCAS | aaacgagttgctatggacagctgacgtacacgaatat | | LLWTADVHEYNLNL | |
| | Positive | aatctgaacttgcctgtcactcccgctgctataacctac | | PVTPAAITYPETAAQ | |
| | Control | ccagaaaccgccgctcagattgccggtgtggttaagt | | IAGVVKCASDYDYK | |
| | | gcgcttctgattacgactataaagtccaagcaaggtcc | | VQARSGGHSFGNYG | |
| | | ggaggtcatagtttcggtaattacggcttgggtggagc | | LGGADGAVVVWDMK | |
| | | tgacggtgcagttgtcgttgatatgaagcacttcactca | | HFTQFSMDDETYEA | |
| | | attttcgatggacgatgaaacttacgaagctgttatcgg | | VIGPGTTLNDVDIEL | |
| | | tccaggtacaactttaaacgatgtcgacatcgaattgta | | YNNGKRAMAHGVC | |
| | | caacaacggtaaaagagccatggctcatggtgtatgt | | PTIKTGGHFTIGGLG | |
| | | ccaaccattaagactggtggtcacttcaccatcggtgg | | PTARQWGLALDHVE | |
| | | tctaggacctacggctcgtcaatggggtctggctttgg | | EVEVVLANSSIVRAS | |
| | | accatgtcgaggaagttgaagttgtgttagctaactcta | | NTQNQDVFFAVKGA | |
| | | gcattgttagagcctctaatacacaaaatcaagatgtttt | | AANFGIVTEFKVRTE | |
| | | ctttgcagtcaagggtgctgctgctaacttcggaatcgt | | PAPGLAVQYSYTFN | |
| | | cactgaatttaaagttagaactgaaccagccccaggtt | | LGSTAEKAQFVKDW | |
| | | tggctgtacagtactcctataccttcaacttgggttcaac | | QSFISAKNLTRQFYN | |
| | | tgccgagaaggctcaattcgttaaggattggcaatcttt | | NMVIFDGDIILEGLF | |
| | | catttcggctaagaacctaaccagacaattttataataa | | FGSKEQYDALGLED | |
| | | catggtcatttttgatggtgacataatcttggaaggtttat | | HFAPKNPGNILVLTD | |
| | | tcttcggtagcaaggaacaatacgacgccttgggcctt | | WLGMVGHALEDTIL | |
| | | gaagatcacttcgcaccaaagaatccaggtaacatatt | | KLVGNTPTWFYAKS | |
| | | ggttttaacagattggctaggcatggtgggtcacgcat | | LGFRQDTLIPSAGID | |
| | | tggaagacactattttaaaattggtcggtaataccccaa | | EFFEYIANHTAGTPA | |
| | | catggttctatgctaagtccttgggttttagacaagaca | | WFVTLSLEGGAIND | |
| | | ctctgatcccttctgccggtattgacgaatttttcgaata | | VAEDATAYAHRDV | |
| | | cattgctaaccataccgccggcactcctgcttggtttgt | | LFWVQLFMVNPVGP | |
| | | tactttgtccttagagggtggtgctatcaacgatgtcgc | | ISDTTYEFTDGLYDV | |
| | | agaagatgctacggcctatgctcacagagatgttttgtt | | LARAVPESVGHAYL | |
| | | ctgggtccaactattcatggttaatccagtcggtcctat | | GCPDPRMEDAQQK | |
| | | ctctgacactacctacgagtttacagacggcttgtacg | | YWRTNLPRLQELKE | |
| | | atgtgttggcccgtgctgttccagaaagcgtgggacat | | ELDPKNTFHHPQGV | |
| | | gcttaccttggttgtccagatccaagaatggaagacgc | | MPA | |
| | | tcaacagaagtattggcgtaccaatttgccccgtctgc | | | |
| | | aagaactaaaggaagagttggatccaaaaaacacctt | | | |
| | | ccatcacccacagggtgttatgccagcttaa | | | |
| t807205 | Library | atgggcaatggacaatccaccccactgcaacagtgttt | 34 | MGNGQSTPLQQCLN | 104 |
| | | aaacacggtatgcaacggtcgtcttggttgtgtcgcttt | | TVCNGRLGCVAFPS | |
| | | cccttcggatgcattgtaccaagccgcttgggtgaagc | | DALYQAAWVKPYN | |
| | | catataatttggacgttcccgttactccaatcgctgtcttt | | LDVPVTPIAVFKPSS | |
| | | aaaccatcttctactgaagacgttgccggtgctattaag | | TEDVAGAIKCAVAS | |
| | | tgtgctgtcgcaagcaacgttcatgttcaagctaagtca | | NVHVQAKSGGHSY | |
| | | ggtggtcacagttacgctaacttcggtttgggtggtca | | ANFGLGGQDGELMI | |
| | | agatggtgagttaatgatagacttggccaatctacaag | | DLANLQDFHMDKTS | |
| | | attttcacatggataaaacctcctggcaggctaccttcg | | WQATFGAGYRLGD | |
| | | gcgctggttacaggttgggtgacctagataagaagttg | | LDKKLQANGNRAIA | |
| | | caagcaaacggaaacagagccattgctcatggtacat | | HGTCPGVGIGGHATI | |
| | | gtccaggtgtaggtatcggaggtcacgctactattggt | | GGLGPMSRMWGSA | |
| | | ggtttaggtcctatgtcaagaatgtggggctctgctctg | | LDHVLSVQVVTADG | |
| | | gatcatgtcttgtccgttcaagtcgttactgccgacggtt | | SIKNASESENSDLFW | |
| | | ctatcaaaaatgcatcagaatctgaaaattctgacttgtt | | ALRGAGASFGVITKF | |
| | | ctgggctttgagaggtgctggtgccagttttggtgtcat | | TVKTHPAPGSVVQY | |
| | | cacaaagttcactgttaagacccacccagccccaggt | | TYKISLGSQAQMAP | |
| | | tccgtggttcaatatacttacaaaatttcgttaggatctc | | VYAAWQALAGDAK | |
| | | aggctcaaatggctcctgtttatgctgcctggcaagca | | LDRRFSTLFIAEPLG | |
| | | ttagctggtgacgctaagttggatagaagattctcaac | | ALITGTFYGTKAEYE | |
| | | cctttttattgctgaaccattgggagccttaataacaggt | | ATGIAARLPSGGTLD | |
| | | actttttacggtacaaaggccgaatatgaagctaccgg | | LKLLDWLGSLAHIA | |
| | | tattgctgcaagacttccatccggcggtaccttggacct | | EVVGLTLGDIPTSFY | |
| | | aaagttattggattggttgggtagcttggctcatatcgct | | GKSLALREEDMLDR | |
| | | gaagttgtcggtctgactttaggtgatattcctacttcttt | | TSIDGLFRYMGDAD | |
| | | ctacggtaaatcgttggccttgagggaagaagacatg | | AGTLLWFVIFNSEG | |
| | | ttggatagaacatccatcgacggtttgtttcgttacatgg | | GAMADTPAGATAY | |
| | | gagatgcagatgctggtacgctattgtggttcgtgatat | | PHRDKLIMYQSYVI | |
| | | tcaactctgagggtggcgctatggccgatactccagct | | GIPTLTKATRDFADG | |
| | | ggtgccactgcttaccctcacagagataagttgattatg | | VHDRVRMGAPAAN | |
| | | tatcaatcttatgtgatcggtattccaacgcttactaaag | | STYAGYIDRTLSREA | |
| | | caactagagactttgctgacggtgtacacgatagagtc | | AQEFYWGAQLPRLR | |
| | | cgtatgggagctccagccgctaacagtacctacgctg | | EVKKAWDPKDVFH | |
| | | gttatatcgacagaaccttatcaagagaagccgctcaa | | NPQSVDPAE | |
| | | gagttttactggggcgctcagttaccaagactaaggg | | | |
| | | aagttaagaaggcttgggaccctaaagacgttttccat | | | |
| | | aatccacaatccgtcgatccagctgaa | | | |
| t807272 | Library | atgggaaatacaacttcaattgcaggcagagattgctt | 35 | MGNTTSIAGRDCLIS | 105 |
| | | gatcagtgctctaggtggtaactctgccttagctgtgttt | | ALGGNSALAVFPNQ | |
| | | cctaaccaacttctgtggacggccgacgtccatgagt | | LLWTADVHEYNLNL | |
| | | ataatttgaacttgccagttactccagctgctataaccta | | PVTPAAITYPETAEQ | |
| | | cccagaaaccgctgaacagattgccggtatcgttaaat | | IAGIVKCASDYDYK | |
| | | gtgcttccgattacgactataaggtccaagctcgttctg | | VQARSGGHSFGNYG | |
| | | gtggtcactcgttcggtaactacggtttaggaggtact | | LGGTDGAVVVDMK | |
| | | gatggcgcagttgtagttgacatgaagcacttcaacca | | HFNQFSMDDQTYEA | |
| | | atttagcatggacgatcaaacctacgaagctgtcattg | | VIGPGTTLNDVDIEL | |
| | | gtcccggtactaccttgaatgatgtagacatcgaattgt | | YNNGKRAMAHGVC | |
| | | ataacaatggtaaaagagctatggcacatggtgtttgt | | PTIKTGGHFTIGGLG | |
| | | ccaactataaagacaggtggacacttcacaattggtg | | PTARQWGLALDHVE | |
| | | gtttaggacctactgccagacaatggggtctagctttg | | EVEVVLANSSIVRAS | |
| | | gaccacgttgaggaagtcgaagttgtcttggctaattc | | NTQNQDVFFAVKGA | |
| | | ctctatcgttagggcttcaaacacccagaaccaagatg | | AADFGIVTEFKVRTE | |
| | | tgttctttgctgtaaagggtgccgctgctgacttcggtat | | PAPGLAVQYSYTFN | |
| | | tgtcacggaatttaaagtcagaactgaaccagcccca | | LGSTAEKAQFVKDW | |
| | | ggtcttgccgtccaatactcttacaccttcaacctaggtt | | QSFISAKNLTRQFYN | |
| | | cgactgctgaaaaggctcaattcgttaaggattggcaa | | NMVIFDGDIILEGLF | |
| | | tctttcatttccgccaagaatttgacgagacaattttataa | | FGSKEQYDALGLED | |
| | | caacatggttatctttgacggtgatattatcttggaaggt | | HFAPKNPGNILVLTD | |
| | | ttattctttggcagtaaagaacaatacgatgcattaggtt | | WLGMVGHALEDTIL | |
| | | tggaagaccatttcgctcccaagaatccaggtaatatc | | KLVGNTPTWFYAKS | |
| | | ttggttttaaccgattggctaggtatggtgggacatgcc | | LGFRQDTLIPSAGID | |
| | | ttagaggacactatattgaagttggttggcaacactcca | | QFFEYIANHTAGTPA | |
| | | acatggttttacgctaaatccttgggtttcaggcaggat | | WFVTLSLEGGAIND | |
| | | actttaattccaagtgctggtatcgatcaatttttcgaata | | VAEDATAYAHRDV | |
| | | cattgctaaccacaccgctggtactcctgcatggttcgt | | LFWVQLFMVNPLGP | |
| | | aaccttgtctctggagggtggtgccatcaatgacgttg | | ISETTYEFTDGLYDV | |
| | | ctgaagacgccactgcttatgctcacagagatgtccta | | LARAVPESVGHAYL | |
| | | ttctgggtccaacttttcatggttaacccattgggtccaa | | GCPDPRMENAPQKY | |
| | | tttctgaaacaacttacgaatttaccgatggattgtacga | | WRTNLPRLQELKEE | |
| | | cgtgctagcacgtgcagttccagaaagcgtcggtcac | | LDPKNTFHHPQGVIP | |
| | | gcttatttgggttgtcctgatccaagaatggagaacgc | | A | |
| | | ccctcaaaagtattggagaacgaatcttccaagacttc | | | |
| | | aagaactgaaggaagagttggatccaaagaacacttt | | | |
| | | tcatcatcctcaaggtgtcatcccagct | | | |
| t807301 | Library | atgggaaacacgaccagcatagctggtcgtgactgtc | 36 | MGNTTSIAGRDCLIS | 106 |
| | | tgatctctgccttgggtggcaattcagcattagctgcttt | | ALGGNSALAAFPNQ | |
| | | cccaaaccaactattgtggactgccgatgtccacgaat | | LLWTADVHEYNLNL | |
| | | acaaccttaatttgcctgtgacaccagctgctattactta | | PVTPAAITYPETAEQ | |
| | | tcccgagactgccgaacagatcgctggtattgttaagt | | IAGIVKCASDYDYK | |
| | | gcgcctctgattacgactacaaagtacaagctagatcg | | VQARSGGHSFGNYG | |
| | | ggtggtcattcctttggtaattatggtttgggtggtaccg | | LGGTDGAVVVDMK | |
| | | atggtgctgtcgttgttgacatgaagcacttcaaccaat | | HFNQFSMDDQTYEA | |
| | | tttctatggatgatcaaacctacgaagcagtcattggac | | VIGPGTTLNDVDIEL | |
| | | caggtactaccttaaacgacgtagatatcgaattgtac | | YNNGKRAMAHGVC | |
| | | aataacggtaaaagagctatggcccatggtgtgtgtcc | | PTIKTGGHFTIGGLG | |
| | | aacaatcaagactggaggtcacttcaccattggcggc | | PTARQWGLALDHVE | |
| | | ttgggtccaactgctagacaatggggtttagctttagac | | EVEVVLANSSIVRAS | |
| | | catgttgaagaggttgaagttgtcttggccaactccagt | | NTQNQDVFFAVKGA | |
| | | attgttagggcatctaatactcaaaaccaggacgttttct | | AADFGIVTEFKVRTE | |
| | | ttgctgtcaagggtgctgctgctgacttcggtatcgtga | | PAPGLAVQYSYTFN | |
| | | ccgaatttaaagttagaacagaacctgccccaggtttg | | LGSTAEKAQFVKDW | |
| | | gccgtccaatattcctacaccttcaatcttggttcaactg | | QYFISAKNLTRQFYN | |
| | | ctgaaaaggcacaattcgtaaaggattggcaatacttc | | NMVIFDGDIILEGLF | |
| | | atctctgctaaaaacctaacaagacaattttacaacaac | | FGSKEQYDALGLED | |
| | | atggttatttttgacggtgatataattttggaaggtctgtt | | HFAPKNPGNILVLTD | |
| | | cttcggtagtaaggaacaatatgacgccttgggtttgg | | WLGMVGHALEDTIL | |
| | | aggatcactttgctcccaagaatccaggaaatattttag | | KLVGNTPTWFYAKS | |
| | | tcctaacggattggttgggcatggttggtcacgcatta | | LGFRQDTLIPSAGID | |
| | | gaagatactattctaaaattggtcggtaacacgccaac | | EFFEYIANHTAGTPA | |
| | | ttggttctatgctaagtccttgggttttcgtcaggacacc | | WFVTLSLEGGAIND | |
| | | cttatcccttctgctggtattgatgaatttttcgagtacat | | VAEDATAYAHRDV | |
| | | cgctaatcataccgccggtactccagcttggtttgttac | | LFWVQLFMVNPLGP | |
| | | tttatctttggaaggtggagctatcaacgacgtcgctga | | ISETTYEFTDGLYDV | |
| | | agatgccacagcatacgcacatagagatgtgttattct | | LARAVPESVGHAYL | |
| | | gggttcaattgttcatggttaaccctcttggtccaatttca | | GCPDPRMENAPQKY | |
| | | gaaacaacttatgaatttaccgatggattgtacgacgtt | | WRTNLPRLQELKEE | |
| | | ttagctagagctgtcccagaatctgtaggtcacgcttac | | LDPKNTFHHPQGVIP | |
| | | ttgggttgtccagacccaagaatggagaacgcacctc | | A | |
| | | aaaagtattggaggacaaacttgccaagactacagga | | | |
| | | actgaaagaggaattggaccccaagaatacttttcacc | | | |
| | | atccacaaggtgttatcccagct | | | |
| t807677 | Library | atggatccaatcgaggacgccattttgcagtgcttaag | 37 | MDPIEDAILQCLSLH | 107 |
| | | cctacacagtgacccttcgcatccaatatcaggcgtaa | | SDPSHPISGVTYFPN | |
| | | cgtatttccccaatacaccatcttacattcctatcctgca | | TPSYIPILHSYIRNLR | |
| | | ctcctacattcgtaaccttagatttacctctccatccacta | | FTSPSTRKPLFIVAPT | |
| | | gaaaaccattgttcatcgttgctccaactcatatatctca | | HISHIQASIICCKSFQ | |
| | | catccaagcatcaattatctgttgtaagtcttttcaattgc | | LQIRIRSGGHDYDGL | |
| | | aaattaggattagaagtggaggtcacgattatgatggtt | | SYVSQSPFAIMDMF | |
| | | tgtcctacgtcagccaatctcccttcgctattatggacat | | AMRSVEVNLEDETV | |
| | | gttcgctatgagatccgttgaagtcaacttagaagatg | | WVDSGSTIGELYHGI | |
| | | aaaccgtttgggttgactctggttccactatcggtgaatt | | AERSKVHGFPAGVC | |
| | | gtaccatggtattgccgaaagatctaaggtccatggttt | | HSVGVGGHFSGGGY | |
| | | cccagctggtgtgtgtcactcagttggcgtcggtggac | | GNMMRKFGLSVDH | |
| | | acttttccggtggtggttatggtaatatgatgagaaagtt | | VLDAVIVDAEGRVL | |
| | | cggtttgtctgtggaccatgttctggatgctgttatcgtt | | DRKKMGEDLFWGIR | |
| | | gatgcagaaggccgtgtcttagacagaaaaaagatg | | GGGGASFGVIVSWR | |
| | | ggtgaagacctattctggggtataagaggtggtggtg | | IKLVPVPEVVTVFRV | |
| | | gcgcttcgtttggtgttatcgtcagttggagaattaaatt | | LKTLEQGATDVVHR | |
| | | ggtcccagtgcctgaggttgtaaccgtcttccgtgtttt | | WQYVADNIHDDLFI | |
| | | gaagaccttggaacaaggtgccacagatgtcgttcac | | RVVLSPVKRKGQKT | |
| | | agatggcaatacgtcgccgacaacatccacgatgact | | IRAKFNALFLGNAQ | |
| | | tatttattagagttgttctatctccagttaagagaaaaggt | | ELLRVMSDSFPELGL | |
| | | cagaagactatcagagctaagtttaatgctttgttcttgg | | VGEDCIEMSWIDSV | |
| | | gtaacgctcaagaattactgcgtgtcatgtctgattctttt | | LFWDNFPVGTSVDV | |
| | | ccagaattgggattagtgggtgaagactgtatcgagat | | LLQRHDTPEKFLKK | |
| | | gagctggattgactccgtattgttctgggataactttcca | | KSDYVQQPISKTGLE | |
| | | gtaggtacatctgtagatgttttattgcagcgtcacgac | | GVWNKMMELEKPV | |
| | | actcctgaaaaattcttgaagaagaaatccgattacgtt | | LTLNPYGGRMGEISE | |
| | | caacaaccaatctctaagactggattagaaggtgtttg | | MEIPFPHRAGNLYKI | |
| | | gaataaaatgatggaacttgaaaagccagtgttgacct | | QYSVNWKEEGEDV | |
| | | tgaatccatatggtggtagaatgggtgaaataagtgaa | | ANRYLDLIRMLYDY | |
| | | atggaaattccttttccacatagagctggtaacttgtaca | | MTPYVSKSPRSSYL | |
| | | agatccaatactcggtcaactggaaggaggaaggtg | | NYRDVDIGVNGPGN | |
| | | aggatgttgcaaacaggtatcttgacctgattagaatgt | | ATYAEARVWGEKY | |
| | | tatacgactacatgaccccatatgtttcaaagtccccca | | FKRNFDRLVEVKTR | |
| | | gatcaagttatttgaactacagagatgtcgatatagga | | VDPSNFFRYEQSIPS | |
| | | gtcaatggtccaggcaatgccacttatgctgaagctag | | LAASSLGIMSE | |
| | | agtctggggagagaaatacttcaagagaaactttgac | | | |
| | | agattggttgaagtcaaaactagggttgatccaagtaa | | | |
| | | cttcttcaggtacgaacaatctataccttccttggccgct | | | |
| | | tcgagcctaggtattatgtcggaa | | | |
| t807764 | Library | atggtccacaatatattactacttggtttgatgccactgtt | 38 | MVHNILLLGLMPLL | 108 |
| | | ggttcgtgcatcacctttgccaatttatcataactacccc | | VRASPLPIYHNYPPQ | |
| | | ccacaatcgactatcaacgactgcttgcaggccgctg | | STINDCLQAADVPAI | |
| | | atgttccagctatcttacaaagctctgcttcctttgatgc | | LQSSASFDALSQPLN | |
| | | cttgagtcaacctctaaattccagattaaaatctaagcc | | SRLKSKPAVITIPTTA | |
| | | agctgtgattacaatccctacgaccgctttgcacgtca | | LHVSSAVKCAAQFK | |
| | | gttctgctgttaagtgtgccgcacaattcaagctgaaa | | LKVTPRGGGHSYNA | |
| | | gtaactccaagaggcggtggacattcttacaacgcac | | QSLGDGAVVIDMQQ | |
| | | aatccttaggtgacggtgctgtcgttattgatatgcaac | | FHDVVYDSKTQLAR | |
| | | agttccacgacgttgtctacgactctaagactcaacta | | IGGGARLGNVAQKL | |
| | | gctaggattggtggtggagctagattgggtaacgttgc | | YDQGKRAMPHGTC | |
| | | ccaaaaattgtatgatcaaggtaagagagctatgccac | | PDVGIGGHSAGGFG | |
| | | atggtacctgtccagatgtcggtattggcggtcactcc | | WTSRQWGITVDHID | |
| | | gccggtggttttggttggacctcacgtcagtggggtat | | EVEVVTADGSIRRA | |
| | | cactgtagatcacatagacgaggttgaagtggtaaca | | NKDQNSDLFWALR | |
| | | gctgacggttctatcagaagagctaataaggatcaaa | | GAAPSFGVITNFWFS | |
| | | attccgatttgttctgggcattgagaggagctgccccat | | TLEAPDSNVIYSYKF | |
| | | cgttcggtgttattactaacttttggttttctaccttggaag | | TGLSLDEISTALLEV | |
| | | ctcctgattctaacgttatttacagttataagttcactggtt | | QKFGQTAPKEVGML | |
| | | tatctttagacgaaatcagtacagctttgttggaagtgc | | IQILDNGSGFRLYGT | |
| | | aaaagttcggtcaaaccgctcccaaagaagtcggcat | | YYNTTRQQFDNLFG | |
| | | gcttatccaaatattagacaatggttctggtttcagattgt | | QLLQRLPSPGNSAEV | |
| | | acggtacgtactataacactacccgtcaacaatttgata | | SVKGWIDSLIFASGG | |
| | | atttattcggccaacttttgcaaagattgccatccccag | | SKGLTVPELGGTNQ | |
| | | gtaacagcgctgaggtttctgtcaagggttggattgac | | HSSFYTKSLMTAQD | |
| | | tcgttgatatttgcctctggcggtagcaagggtcttact | | YPLTLDSIKSVFKYA | |
| | | gttccagaactgggtggaactaaccagcattcttccttt | | MNQGRAATERGLP | |
| | | tacacaaaatcattgatgactgctcaagattacccatta | | WMVFISLLGGRYST | |
| | | accctggattcaattaagtccgtgttcaagtatgccatg | | LPTPSAASDNSFYGR | |
| | | aaccaaggtagagccgccaccgaaaggggtctacc | | NTLWAFSFTAYLGN | |
| | | atggatggtatttatctctttgttgggtggtagatatagc | | VTEQSNRDSIYFLNG | |
| | | actctaccaacgccttccgctgcttcagataactctttct | | FDTSVRRSVDTAYIN | |
| | | acggcagaaacactttgtgggctttttctttcaccgctta | | GHDTEYSREEAHRL | |
| | | cctaggtaacgtcacagaacaaagcaatagagactca | | YYGDKYQRLSVLKK | |
| | | atttacttcttgaatggtttcgacacttccgtaagaagat | | QWDPEQVFWYPQSI | |
| | | ccgttgacaccgcttacatcaacggtcacgatactgaa | | DPAN | |
| | | tattcgagagaagaagcacatagattatactacggtga | | | |
| | | caaatatcaaaggttgtctgtcttaaagaagcaatggg | | | |
| | | atcctgagcaagttttctggtatccacaatccatcgacc | | | |
| | | ccgccaat | | | |
| t807774 | Library | atgggtaacacaacttcaatcgcagctggcagggatt | 39 | MGNTTSIAAGRDCL | 109 |
| | | gcttactgtccgccgtcggaggtaatcacgctcatgtt | | LSAVGGNHAHVAFQ | |
| | | gcttttcaggaccaattgctatatcaagctaccgcagtg | | DQLLYQATAVEPYN | |
| | | gaaccatacaacttgaatattcccgttacgccagccgc | | LNIPVTPAAVTYPQS | |
| | | tgttacctaccctcaatcggctgatgaggttgccgctgt | | ADEVAAVVKCAAD | |
| | | cgtaaaatgtgcagccgactatggttacaaggtgcaa | | YGYKVQARSGGHSF | |
| | | gctagaagcggtggtcacagtttcggtaactacggttt | | GNYGLGGEDGAIVV | |
| | | aggcggtgaagacggtgctatagtcgttgatatgaag | | DMKHFDQFSMDEST | |
| | | catttcgatcaattttctatggacgaatctacttatactgc | | YTATIGPGITLGDLD | |
| | | tactattggtccaggtatcaccttgggagacttggatac | | TALYNAGHRAMAH | |
| | | cgccctatacaatgctggccatagagccatggctcac | | GICPTIRTGGHLTIGG | |
| | | ggtatttgtccaacaattcgtactggtggtcaccttacc | | LGPTARQWGLALDH | |
| | | atcggaggtttgggtccaactgctagacaatggggttt | | VEEVEVVLANSSIVR | |
| | | ggccttagatcacgttgaagaagtcgaagttgtcttgg | | ASDTQNQEILFAVK | |
| | | caaacagctccatcgtcagagcatcagacactcagaa | | GAAASFGIVTEFKVR | |
| | | ccaagagatcttgttcgctgttaagggtgctgctgcttc | | TEEAPGLAVQYSFTF | |
| | | tttcggtatagtaactgaatttaaagttagaacagaaga | | NLGTAAEKAKLVKD | |
| | | agctcctggtcttgccgtccaatactccttcaccttcaa | | WQAFIAQEDLTWKF | |
| | | cttaggtacagctgccgagaaggctaaattggttaag | | YSNMNIIDGQIILEGI | |
| | | gactggcaagcttttattgcacaagaagatttgacgtg | | YFGSKAEYDALGLE | |
| | | gaagttctactctaacatgaatattatcgacggtcaaatt | | EKFPTSEPGTVLVLT | |
| | | atcctagaaggcatatatttcggttctaaggctgaatac | | DWLGMVGHGLEDV | |
| | | gatgccttaggtttggaggaaaagtttccaaccagtga | | ILRLVGNAPTWFYA | |
| | | accaggcactgttttagtcttgacggactggctgggtat | | KSLGFAPRALIPDSAI | |
| | | ggttggtcatggtttggaagatgttattttgcgtttagtag | | DDFFEYIHKNNPGT | |
| | | gcaatgctccaacttggttctatgctaaatctttaggtttt | | VSWFVTLSLEGGAI | |
| | | gctcccagggcattgatcccagattccgctattgacga | | NKVPEDATAYGHRD | |
| | | tttcttcgaatacattcacaagaacaatcctggtaccgtt | | VLFWVQIFMINPLGP | |
| | | agttggttcgtcacactatcgttggaaggtggtgcaata | | VSQTIYDFADGLYD | |
| | | aacaaggtgccagaagatgccactgcttacggacata | | VLAKAVPESAGHAY | |
| | | gagatgttttgttttgggttcaaatctttatgattaaccca | | LGCPDPRMPNAQQA | |
| | | ctaggtcctgtttctcagaccatttacgactttgccgac | | YWRNNLPRLEELKG | |
| | | ggtctttatgacgttctggctaaagccgtccccgaatcc | | DLDPKDIFHNPQGV | |
| | | gcaggtcatgcttatttgggctgtccagacccaagaat | | MVVS | |
| | | gccaaatgctcaacaagcctactggagaaataacttg | | | |
| | | ccaagactagaggaattgaagggtgacttagatccaa | | | |
| | | aggatatcttccacaacccacagggtgtcatggttgtct | | | |
| | | ct | | | |
| t807810 | Library | atgggtaacactaccagcattgccggccgtgactgcc | 40 | MGNTTSIAGRDCLV | 110 |
| | | tagtttccgctttgggtggtaatgcaggtctggtggcttt | | SALGGNAGLVAFQS | |
| | | tcagtcacaaccattataccaaacaaccgctgtccatg | | QPLYQTTAVHEYNL | |
| | | agtataaccttaacatacccgttactccagccgctatcg | | NIPVTPAAIAYPETA | |
| | | cttaccctgaaactgccgaacaaattgctgctgtcgta | | EQIAAVVKCASEYD | |
| | | aaatgtgcatcggaatatgattacaaggttcaagcaag | | YKVQARSGGHSFGN | |
| | | atccggtggtcactctttcggaaattacggtttgggtgg | | YGLGGTDGAVVVD | |
| | | tacggatggtgctgttgtggtcgacatgaagcacttca | | MKHFNQFSMDDQT | |
| | | accaatttagtatggacgatcaaacctatgaagctgtta | | YEAVIGPGTTLGDV | |
| | | tcggcccaggtactactttgggcgacgtcgatactga | | DTELYNNGKRAMA | |
| | | gctatacaataacggtaagagagccatggcccatggt | | HGICPTISTGGHFTM | |
| | | atctgtccaacaatttctaccggtggccacttcacgatg | | GGLGPTARQWGLAL | |
| | | ggtggtttaggtccaacggctagacagtggggtttgg | | DHVEEVEVVLANSSI | |
| | | cattggatcacgttgaagaagtagaagtcgttttggcta | | VRASNTQNQEVFFA | |
| | | attcttctatcgtgagggcttccaacacccaaaaccaa | | VKGAAASFGIVTEF | |
| | | gaagttttctttgccgttaaaggagctgctgcttcatttg | | KVRTQPAPGLAVQY | |
| | | gtattgtcaccgaatttaaggttagaactcaaccagctc | | SYTFNLGSSAEKAQF | |
| | | ctggattggctgtccaatactcttacactttcaacttggg | | VKDWQSFISAKNLT | |
| | | ttcgagtgctgaaaaggctcaattcgtcaaggattggc | | RQFYTNMVIFDGDII | |
| | | aatctttcatctctgctaaaaacttaacaagacagttttat | | LEGLFFGSKEQYEAL | |
| | | accaatatggttatattcgacggcgacattattttggaa | | GLEERFVPKNPGNIL | |
| | | ggtctgttctttggtagcaaggagcaatacgaagccct | | VLTDWLGMVGHAL | |
| | | tggtttggaagaacgtttcgtcccaaagaatcctggta | | EDTILRLVGNTPTWF | |
| | | acattcttgttttaactgattggttgggtatggttggtcat | | YAKSLGFTPDTLIPS | |
| | | gctttggaggacactatcttaagattagtcggtaacacc | | SGIDEFFEYIENNKA | |
| | | ccaacctggttctacgcaaaatccctaggcttcacccc | | GTSTWFVTLSLEGG | |
| | | agatactttgataccctcctcaggtattgatgaatttttcg | | AINDVPADTTAYGH | |
| | | aatatatcgagaataataaggccggtacctctacatgg | | RDVLFWVQIFMVSP | |
| | | tttgtaacattatctcttgaaggtggtgccatcaacgac | | TGPVSSTTYDFADG | |
| | | gttccagctgatacgacagcatacggtcacagagatg | | LYNVLTKAVPESEG | |
| | | tattgttttgggtccagatattcatggtttccccaactggt | | HAYLGCPDPKMAN | |
| | | ccagtttcctctacaacttacgattttgctgacggcttgt | | AQQKYWRQNLPRL | |
| | | ataacgtgttgactaaggcagttcctgaaagcgaaggt | | EELKATLDPKDTFH | |
| | | catgcttacttgggatgtcctgaccctaagatggctaac | | NPQGILPV | |
| | | gcccaacaaaaatattggagacaaaatctaccaagac | | | |
| | | tggaggaattgaaagctactcttgacccaaaggatac | | | |
| | | ctttcataacccccaaggtatcttgccagta | | | |
| t807822 | Library | atgaatccttctataccctcaagctccatgggtaacaca | 41 | MNPSIPSSSMGNTTS | 111 |
| | | acgtctatcgctggacgtgactgtttagttagtgccctg | | IAGRDCLVSALGGN | |
| | | ggtggtaacgctggtttggtagcattccaaaatcagcc | | AGLVAFQNQPLYQT | |
| | | actataccaaaccactgctgtgcacgagtataacttaa | | TAVHEYNLNIPVTPA | |
| | | acattccagtcactccagccgctattacctacccagaa | | AITYPETAEQIAAVV | |
| | | actgctgaacaaatcgccgctgttgtcaaatgcgcatc | | KCASQYDYKVQARS | |
| | | ccaatatgattacaaggttcaagctaggtctggtggcc | | GGHSFGNYGLGGTD | |
| | | attcgtttggtaactacggtcttggtggcaccgatggtg | | GAVVVDMKYFNQF | |
| | | ctgttgtcgttgacatgaagtatttcaatcaattttccatg | | SMDDQTYEAVIGPG | |
| | | gacgatcagacatacgaagcagttattggtcctggtac | | TTLGDVDVELYNNG | |
| | | taccttgggagatgtcgatgtcgaattgtataacaatgg | | KRAMAHGVCPTIST | |
| | | taaaagagctatggcccacggtgtgtgtccaactatct | | GGHFTMGGLGPTAR | |
| | | ctaccggtggccatttcactatgggtggtttaggtccaa | | QWGLALDHVEEVE | |
| | | cagctagacaatggggattggccttggaccacgttga | | VVLANSSIVRASNTQ | |
| | | ggaagttgaagtggttctagctaattcatctatcgtcag | | NQEVFFAVKGAAAS | |
| | | agcttcaaacacccaaaaccaagaagttttctttgccgt | | FGIVTEFKVRTQPAP | |
| | | aaagggtgctgctgcctcgtttggtattgtcaccgaatt | | GIAVQYSYTFNLGSS | |
| | | taaggttagaactcagcctgcaccaggtattgctgtgc | | AEKAQFIKDWQSFV | |
| | | aatactcttacactttcaacttgggttcctccgcagaaa | | SAKNLTRQFYTNMV | |
| | | aagctcaattcatcaaggactggcaatctttcgtttctgc | | IFDGDIILEGLFFGSK | |
| | | taagaatcttacgagacaattctacactaacatggtcat | | EQYEALGLEERFVP | |
| | | atttgacggtgatattattttggaaggattgttcttcggta | | KNPGNIMVLTDWLG | |
| | | gtaaagagcaatatgaagccttgggtttagaagaaag | | MVGHALEDTILRLV | |
| | | gtttgtccctaagaacccaggtaatatcatggttctaac | | GNTPTWFYAKSLGF | |
| | | agattggttgggtatggttggccatgctctggaagata | | TPDTLIPSSGIDEFFE | |
| | | cgattttgagattggtaggtaatacgccaacttggttcta | | YIENNKAGTSTWFV | |
| | | cgctaagtccctgggttttactccagacacattaatccc | | TLSLEGGAINDVPAD | |
| | | cagcagcggtattgacgaatttttcgaatatatagaaaa | | ATAYGHRDVLFWV | |
| | | caacaaggcaggaaccagtacttggttcgtcaccctat | | QIFMVSPTGPVSSTT | |
| | | ctttggagggtggtgctattaacgatgtgccagcagat | | YDFADGLYNVLTKA | |
| | | gctaccgcttacggtcacagagatgttttattctgggta | | VPESEGHAYLGCPD | |
| | | cagatttttatggtttctcccactggtccagtttcctccac | | PKMANAQQKYWRQ | |
| | | tacctacgactttgccgacggcttgtacaatgtcttgac | | NLPRLEELKETLDPK | |
| | | taaggctgttccagagtctgaaggccacgcctatttgg | | DTFHNPQGILPA | |
| | | gctgtccagaccctaaaatggctaacgctcaacaaaa | | | |
| | | gtactggagacaaaacttgccacgtctggaagagctt | | | |
| | | aaggaaacattagatccaaaagacactttccacaatcc | | | |
| | | tcaaggtatcttgccagca | | | |
| 1807854 | Library | atggcccgtgtccccgcactgttcttgggcctatcttca | 42 | MARVPALFLGLSSV | 112 |
| | | gtgtgtttgttagttttcttttgcagtttgattaacccaacc | | CLLVFFCSLINPTTPL | |
| | | actcctttacatcagcacggtaagcaaacatctatcgct | | HQHGKQTSIAGRDC | |
| | | ggtagggattgtcttgtatccgctttgggtggaaatact | | LVSALGGNTGLVAF | |
| | | ggtttagttgcttttcaagaccaattgttgtaccaaacca | | QDQLLYQTTAVHEY | |
| | | cggctgtccacgagtataacctgaacataccagttact | | NLNIPVTPAAITYPE | |
| | | ccagccgctatcacctacccagaaacctcggaacaa | | TSEQIAAVVKCASE | |
| | | attgctgccgttgttaaatgtgcatctgaatacgattata | | YDYKVQARSGGHSF | |
| | | aggtccaggccagatccggtggtcatagcttcggtaa | | GNYGLGGADGAVV | |
| | | ttacggattgggtggtgctgacggtgctgttgtcgttga | | VDMKHFTQFSMDE | |
| | | tatgaagcacttcactcaattttctatggacgaacaaac | | QTYEAVIGPGTTLG | |
| | | ctatgaagctgtaattggcccaggtacaactctaggtg | | DVDTELYNNGKRA | |
| | | atgtggataccgagttgtacaacaacggtaaaagagc | | MAHGICPTISTGGHF | |
| | | tatggcccatggtatctgtcctactatctccactggcgg | | TMGGLGPTARQWG | |
| | | tcacttcaccatgggtggtcttggcccaacagcaaga | | LALDHVEEVEVVLA | |
| | | caatggggattggctcttgaccacgtcgaagaagtgg | | NSSIVRASNTQNQEV | |
| | | aagttgttttggctaattcctctattgtcagagcatcaaa | | FFAVKGAAASFGIVT | |
| | | cactcaaaatcaagaagttttctttgctgttaagggtgct | | EFKVRTQPAPGLAV | |
| | | gccgcctccttcggtatcgtcactgaatttaaagtcaga | | QYSYTFNLGSSAEK | |
| | | actcaaccagccccaggattagctgttcaatactcttat | | AQFVKDWQSFISAK | |
| | | acgtttaacttgggtagctcagctgagaaggctcaatt | | NLTRQFYTNMVIFD | |
| | | cgtcaaggactggcaatctttcatttctgctaagaattta | | GNIILEGLFFGSKEQ | |
| | | accagacagttttacacaaacatggtaattttcgatggt | | YEALGLEDRFVPKN | |
| | | aacataatcctagaaggcctatttttcggttccaaagaa | | PGNILVLTDWLGMV | |
| | | caatacgaagctttgggtttggaggaccgttttgttcca | | GHALEDTILRLVGN | |
| | | aagaatcctggtaatatcttagtcttgacagattggcta | | TPTWFYAKSLGFTP | |
| | | ggtatggttggccatgcattggaggacactattttgag | | DTLIPSSGIDEFFDYI | |
| | | attggtcggtaacacccccacatggttctacgcaaaga | | ENNKAGTLTWFVTL | |
| | | gcttgggtttcactccagacacgttaatacctagttcag | | SLEGGAINDVPKDA | |
| | | gtattgatgaatttttcgattatatagaaaacaacaaggc | | TAYGHRDVLFWVQI | |
| | | tggtaccctgacgtggttcgttactttgagtttagaaggt | | FMASPTGPVSSTTYD | |
| | | ggtgccattaacgatgtgccaaaggatgctactgctta | | FADGLYNVLTKAVP | |
| | | tggtcacagagatgtgcttttttgggttcagatcttcatg | | ESEGHAYLGCPDPK | |
| | | gcttctccaacaggtcctgtctcttccactacctacgac | | MADAQQKYWRQNL | |
| | | ttcgccgatggtttgtataatgtcttgaccaaagccgttc | | PRLEELKATLDPKDT | |
| | | cagaatccgaaggacatgcttacctgggttgtccaga | | FHNPQGILPA | |
| | | cccaaagatggctgacgctcaacaaaagtactggag | | | |
| | | gcaaaacttacctagacttgaagaattgaaagcaacct | | | |
| | | tagatccaaaagatacttttcacaatccacaaggtatctt | | | |
| | | gccagca | | | |
| t807859 | Library | atgacaaccaagtgtaggcatgcagtggaggctactg | 43 | MTTKCRHAVEATAL | 113 |
| | | ctctaggcacgatggtagcctacctgattagaaataac | | GTMVAYLIRNNPRR | |
| | | ccacgtagacctagttcaactaacgctaatgttttggac | | PSSTNANVLDTGLG | |
| | | acaggtttgggtggtgccgatggtgctgttgtcgtcga | | GADGAVVVDMKHF | |
| | | tatgaaacactttactcagttctccatggacgatgaaac | | TQFSMDDETYEAVI | |
| | | ctatgaagctgttatcggaccaggtaccactttaaacg | | GPGTTLNDVDIELY | |
| | | acgttgatatagaattgtacaacaacggtaagagagct | | NNGKRAMAHGVCP | |
| | | atggcacacggtgtctgccccaccattaagactggtg | | TIKTGGHFTIGGLGP | |
| | | gtcatttcaccatcggcggacttggtccaactgccaga | | TARQWGLALDHVE | |
| | | caatggggtttggccttagatcacgttgaagaggttga | | EVEVVLANSSIVRAS | |
| | | agtcgtgttggctaattcgtctattgtaagagctagcaa | | NTQNQDVFFAVKGA | |
| | | cacacaaaaccaagacgttttctttgcagttaaaggtg | | AANFGIVTEFKVRTE | |
| | | ctgccgctaatttcggaatcgtcactgaatttaaggtca | | PAPGLAVQYSYTFN | |
| | | gaaccgaaccagctcctggtttggctgttcaatattcct | | LGSTAEKAQFVKDW | |
| | | acactttcaatttaggttctactgctgaaaaggctcaatt | | QSFISAKNLTRQFYN | |
| | | cgtgaaagattggcaatcttttatctctgctaagaatcta | | NMVIFDGDIILEGLF | |
| | | acgagacagttctacaacaacatggttatttttgacggt | | FGSKEQYDALGLED | |
| | | gatatcattcttgagggtttgtttttcggttccaaggaac | | HFAPKNPGNILVLTD | |
| | | aatatgacgccttgggcttggaagatcatttcgctccaa | | WLGMVGHALEDTIL | |
| | | agaatccaggtaacatcttagtcctgacagactggcta | | KLVGNTPTWFYAKS | |
| | | ggtatggttggacacgccttggaagacaccattttgaa | | LGFRQDTLIPSAGID | |
| | | gcttgttggtaacaccccaacctggttctacgccaaat | | EFFEYIANHTAGTPA | |
| | | cgttgggttttagacaagatacattaattccatcagctg | | WFVTLSLEGGAIND | |
| | | gtatagacgaatttttcgaatacatagctaaccacactg | | VAEDATAYAHRDV | |
| | | ctggtactcccgcatggttcgtcacacttagtttggaag | | LFWVQLFMVNPLGP | |
| | | gtggtgctattaatgatgtcgcagaagatgccacggc | | ISDTTYEFTDGLYDV | |
| | | atatgctcaccgtgacgtcttgttctgggtacaactgttt | | LARAVPESVGHAYL | |
| | | atggtgaaccctttgggtcccatctctgacacgacttac | | GCPDPRMEDAQQK | |
| | | gaatttactgacggtttatatgatgttttggcaagagctg | | YWRTNLPRLQELKE | |
| | | ttccagagtccgtgggccatgcttacttaggttgtccag | | ELDPKNTFHHPQGV | |
| | | atcctagaatggaagatgctcagcaaaagtattggcgt | | MPA | |
| | | accaacttgccaaggttgcaagagttgaaagaagaac | | | |
| | | tagacccaaagaacactttccaccatccacaaggtgtt | | | |
| | | atgccagcc | | | |
| t807860 | Library | atgggtcatggacaatccactcccggtgagaactgttt | 44 | MGHGQSTPGENCLN | 114 |
| | | aaatgcaatatgcggtaacagaacggactgtgtatcat | | AICGNRTDCVSYPL | |
| | | acccattggatcctctattccacatcgcttgggcccgtc | | DPLFHIAWARPYNL | |
| | | catataaccttcaggtgcctgttacaccagctgctgtcc | | QVPVTPAAVLRPDN | |
| | | tgaggccagacaatgctcaagatgttgctgatgccgtt | | AQDVADAVKCANE | |
| | | aaatgtgccaacgaaaatggcattaaggtccaagcta | | NGIKVQARSGGHSY | |
| | | gatctggtggtcactcgtacggtaactttggtttgggag | | GNFGLGGDDGALVL | |
| | | gtgacgacggtgcattggttttggatctagtcaatttac | | DLVNLQGFTMDEEN | |
| | | aaggtttcactatggacgaagaaaactggcatgctgct | | WHAAVGAGIRLGKL | |
| | | gttggcgcaggtattagattgggtaagctggatgaaca | | DEHLHKNGGRAMA | |
| | | cttgcataaaaatggtggtagagctatggctcacggta | | HGTCPGVGIGGHATI | |
| | | cctgtccaggcgtaggtatcggtggtcacgccaccat | | GGLGPMSRMWGSA | |
| | | cggaggtttaggtccaatgagcagaatgtggggttct | | LDHVVEVEVVTADG | |
| | | gccttggatcatgtcgtcgaagttgaagttgttactgct | | SIQRANETENSDLFW | |
| | | gacggctctattcaacgtgctaacgaaacagaaaata | | ALRGAGASFGIITEF | |
| | | gtgatttattttgggccttgagaggtgctggtgcttcctt | | VVRTHPEPGDVIEYT | |
| | | cggtataatcactgagtttgtagttagaacccacccag | | YSLTFGSQAEMAPIY | |
| | | aaccaggagatgtaattgaatatacctactctctgacct | | QEWQEFIGDPDLDR | |
| | | tcggttcacaagcagaaatggctcctatttaccaagaa | | RFSSQFIAQPLGAIIT | |
| | | tggcaggaatttattggtgacccagacttggatagaag | | GTFYGTEDEYRESGI | |
| | | gttctcctctcaattcattgctcaaccattgggtgctatc | | PDKIPGGGSDGVGIL | |
| | | atcactggaaccttctacggtactgaagacgaatatag | | VTDWLGSLGHQAER | |
| | | agagagtggtatccccgataagataccaggaggtggt | | SGLAISDLPSPFYSKS | |
| | | agtgatggtgttggtattttagttacagactggttgggtt | | LAFRKEDLLPKEGIT | |
| | | ctctaggccaccaagccgaaagatccggtcttgctatt | | ELFQYLNDADHGTL | |
| | | tcggatcttccaagccctttttattctaagtcgttggcctt | | IWVVIFNSEGGAMG | |
| | | cagaaaggaagacttgttgccaaaagagggtataact | | DTAANATAYPHRDK | |
| | | gagttattccagtacttgaacgacgctgaccatggtac | | TMMYQSYVIGIPEVS | |
| | | attgatctgggtcgtcatctttaactccgaaggaggtgc | | GTARRFLEGVHAKI | |
| | | aatgggtgatactgccgcaaacgctacagcctaccca | | QDAAPGANSTYAGY | |
| | | catcgtgataagaccatgatgtatcaatcctacgtcatt | | IDTELGRAEAQEVY | |
| | | ggtatccccgaagtgagtggcaccgcaagaagattc | | WGSQLPQLRKIKKD | |
| | | ctggagggtgttcacgctaagattcaagatgctgctcc | | WDPKDRFSNPQSVQ | |
| | | tggtgctaattcaacgtatgctggttacatcgatacgga | | AAR | |
| | | attaggcagagctgaagctcaagaagtgtactggggt | | | |
| | | agccagttgcctcaattgagaaagatcaaaaaggact | | | |
| | | gggacccaaaggacaggttttcaaacccacaatctgt | | | |
| | | ccaagccgccaga | | | |
| t807861 | Library | atgcgtgtcgttggaaagatgggtgctttgcaaagcac | 45 | MRVVGKMGALQST | 115 |
| | | tctggagaaatctatcaaggccgcattagctggtgacg | | LEKSIKAALAGDDD | |
| | | atgatctatacgctgtgcccggtaaaccattttatcagat | | LYAVPGKPFYQIQH | |
| | | acaacatgtcaagccttacaacttgtcgattccaatcga | | VKPYNLSIPIEPAAIT | |
| | | accagccgctattacctatcctaagacaactgctcaag | | YPKTTAQVAAIIKCA | |
| | | tagccgcaattatcaagtgcgctgttgctgctaatttga | | VAANLKVQARSGG | |
| | | aggtccaagccagatcaggtggccactcctacgctaa | | HSYANYCIGGVSGA | |
| | | ctactgtattggtggtgtttctggtgctgttgttatcgacc | | VVIDLKHFQRFSMD | |
| | | ttaaacacttccaaagattcagtatggatagaaccacgt | | RTTWQAAVGAGTL | |
| | | ggcaagcagccgtcggtgctggcactttattgggtaat | | LGNLTKRMHEAGN | |
| | | ttgaccaagaggatgcatgaagctggtaacagagcc | | RAMAHGTCPQVGIG | |
| | | atggctcacggtacttgtccacaagtgggaattggtgg | | GHATIGGLGPSSRL | |
| | | tcacgcaaccataggtggccttggtccatcttcaagatt | | WGTALDHVEEVEIV | |
| | | gtggggtacggctttagaccatgttgaagaagtcgaa | | LADSTIKRCSATQNP | |
| | | atagtcttggctgattccacaattaagagatgttctgcta | | DIFWAVKGAGASFG | |
| | | ctcagaatccagacatcttttgggccgttaagggagct | | VVTEFKLRTEPEPSE | |
| | | ggtgcatccttcggtgttgtgactgaatttaaattaagaa | | AVHFSYSFTVGSYA | |
| | | ccgagcccgaaccatctgaagctgtacatttctcttatt | | SLAAVFKSWQSFVA | |
| | | cgttcactgttggttcctacgcaagcttggctgctgttttt | | DPGLTRKFSSEVIITE | |
| | | aaatcatggcaatctttcgtcgctgacccaggtcttact | | IGMIISGTYFGSQAE | |
| | | cgtaagttctcctctgaagtcatcattacagagatcggt | | YDALDMKSQLRGDS | |
| | | atgattatatcaggcacttattttggtagtcaagctgaat | | VAKIIVFKDWLGLL | |
| | | acgatgccctagatatgaagtctcaattgagaggtgac | | GHWAEDVGLRIAGG | |
| | | agtgttgctaagatcattgtttttaaggactggttaggatt | | LPAPLYAKTLTFNG | |
| | | gttgggtcactgggccgaagatgtgggcctaagaatt | | ANLIPDEVIDKLFAY | |
| | | gccggtggtttacctgcccctttgtacgctaaaaccttg | | LDKVEKGALVWFVI | |
| | | accttcaacggtgccaacctgatcccagatgaagtcat | | FDLAGGAVNDIAQD | |
| | | cgataaattgttcgcctacctggacaaggttgaaaagg | | ATSYAHRDALFYLQ | |
| | | gagctttggtatggttcgtcatttttgacctggctggag | | SYAVGLGNVSQTTK | |
| | | gtgccgttaatgacatagctcaagatgctacatcctatg | | DFLTGINTTITNGMP | |
| | | ctcatcgtgatgccttgttctacttgcagtcatatgcagt | | EGGDFGAYPGYVDL | |
| | | gggtttaggtaacgtttcacaaacaactaaggattttctt | | ELPNGPHAYWRTNL | |
| | | accggtataaacacgactattaccaacggtatgccag | | PRLEQIKALVDPND | |
| | | aaggtggtgacttcggtgcttacccaggctacgttgac | | VFHNPQSYLCILFLL | |
| | | ttggaattaccaaatggtccacacgcttactggagaac | | NLLNRALAWAPVGT | |
| | | caaccttccaaggttggaacaaatcaaagccctggta | | VQPFQVLRYSIDTGP | |
| | | gatcctaatgatgtcttccacaacccacaatcttatttgt | | LVLL | |
| | | gcatcctatttttgctaaacttgctaaacagagctttggc | | | |
| | | ttgggctccagttggtactgtccagccattccaagtctt | | | |
| | | aaggtactccattgacacaggtcctcttgtgcttttg | | | |
| t807863 | Library | atgggtcagggctcgagcggtgtgcaatctaacccct | 46 | MGQGSSGVQSNPLE | 116 |
| | | tagaagattgtttgaaggtagctacaagtccactaggtt | | DCLKVATSPLGSYA | |
| | | catacgccttccatgacaaattgctgtttcaacttaccg | | FHDKLLFQLTDVKP | |
| | | atgttaagccttataatttagactacccagtcaacccaat | | YNLDYPVNPIAVTY | |
| | | cgctgttacgtatccaggttccactaaagaggttgcac | | PGSTKEVAQIIKCAT | |
| | | aaattataaagtgcgctaccacttacgataagaaggtc | | TYDKKVQARSGGHS | |
| | | caagccagaagcggaggtcactcttacgctaatttcg | | YANFALGDGDGAIV | |
| | | ctttgggtgacggtgacggtgcaattgttatcgatatgc | | IDMQKFKQFSMDTS | |
| | | aaaaatttaagcaattctccatggacacttctacctggc | | TWQATIGPGTLLGD | |
| | | aggctacaattggtcctggtactttgttgggtgatgtctc | | VSKRLHENGNRVIP | |
| | | caagcgtttacacgaaaacggtaacagggtaatccca | | HGTSPQIGFGGHGTI | |
| | | catggaacctctccacaaataggtttcggaggccacg | | GGLGPLSRMYGLTL | |
| | | gtactattggtggtctgggccctttgtctcgtatgtacgg | | DSIEEVEAVLANGQI | |
| | | tttaaccttggactccatcgaagaagttgaagccgtctt | | VRASKTQNEDLFFAI | |
| | | ggctaacggtcaaattgttagagctagtaaaactcaaa | | RGAAASVAVVTEFK | |
| | | atgaagatctattttttgctattagaggagccgccgcttc | | VRTYPEPSSSVLYSY | |
| | | agtcgcagttgtcacagaatttaaggttagaacctatcc | | TLQGGSVASRANAF | |
| | | agagccctctagttctgtgttatattcttacactttacaag | | KQWQKLTTDPSVSR | |
| | | gtggttcagttgcttccagagctaacgctttcaagcagt | | KFASTFVLSEAITVV | |
| | | ggcaaaaattgacgacagatccatcggtcagcagaa | | TGTFFGTQAEFDSLD | |
| | | agttcgcttctactttcgttctatccgaagccataaccgt | | ITSRLPADMISNNTE | |
| | | cgtcacgggtactttcttcggtactcaagctgagtttgat | | VKNWLGVVGHWGE | |
| | | tccttggacatcacctctaggttgcctgccgacatgatc | | SLALRAGGGIPAHFY | |
| | | tccaataatacagaagttaagaactggttgggtgtcgtt | | SKSLGFKKDEIMDD | |
| | | ggccattggggtgaatcattggctttgagagccggtg | | ATVDKLFNYIDKAD | |
| | | gtggtattccagcacacttttactccaagtctttgggtttc | | KGGAVWFVIWDLE | |
| | | aaaaaggatgagatcatggatgatgctactgtggaca | | GGAISDVPTTETSYG | |
| | | agctattcaattatattgacaaagctgataaaggaggtg | | HRDAIFFQQSYAINL | |
| | | ctgtttggttcgttatttgggaccttgaaggaggtgctat | | LGRVKDDTHEFLNR | |
| | | ctctgatgttccaaccactgaaacttcttacggtcatag | | VNSVIMESNPGGYW | |
| | | agatgcaatctttttccaacagtcttatgcaattaacttat | | GAYPGYVDTALGNS | |
| | | tgggtagagttaaggacgacacccacgaatttttgaac | | SAKAYWGINSERLQ | |
| | | agagttaatagtgtaattatggaatctaacccaggtggt | | TIKSWVDAGDVFHN | |
| | | tactggggtgcctacccaggttatgtcgatactgctcta | | PQSVRPK | |
| | | ggtaattccagcgctaaggcctactggggtatcaaca | | | |
| | | gcgaaagattacaaaccataaaaagttgggtagacgc | | | |
| | | tggtgatgtgtttcacaacccacaatcagttagaccca | | | |
| | | ag | | | |
| t807866 | Library | atgcagccttttacaagccttactaggtcccccttccgtt | 47 | MQPFTSLTRSPFRSA | 117 |
| | | cagcccacgttatcagttgtccagtcgctttggacaatc | | HVISCPVALDNPPSV | |
| | | caccatcggtaccaattataatgggacaaaagccttcc | | PIIMGQKPSSPLATC | |
| | | tctccattagctacctgcttggataaagtttgtaacggta | | LDKVCNGRSSCVGY | |
| | | gatctagttgtgtcggttacccaaacgaccccctattcc | | PNDPLFQINWVKPY | |
| | | aaatcaattgggttaagccatataacttggatattcctgt | | NLDIPVQPIAVTRPS | |
| | | ccaaccaattgcagtgactagaccatctaccgctgag | | TAEDVAGFVKCAAE | |
| | | gatgttgccggttttgttaagtgtgctgctgaaaacaat | | NNVKVQAKSGGHS | |
| | | gtcaaagtccaagcaaagtctggcggtcattcctacg | | YGNFAIGGTDGALVI | |
| | | gtaacttcgctatcggtggtactgacggtgccttagtta | | DLVNFQNFSMDTNT | |
| | | ttgatctggtgaattttcaaaacttcagcatggatacaaa | | WQATFGGGHKLHE | |
| | | cacctggcaggctacgttcggtggaggccacaagttg | | VTQKLHDNGKRAIA | |
| | | catgaagttactcaaaaactacacgacaatggtaaga | | HGTCPGVGIGGHATI | |
| | | gagctatcgcccacggtacctgtccaggtgttggtata | | GGLGPSSRMWGSCL | |
| | | ggtggacatgctactattggtggtttgggtccatcttctc | | DHVVEVEVVTADG | |
| | | gtatgtggggctcctgcttggatcacgtagttgaagtc | | KIQRANDKQNSDLF | |
| | | gaagtcgttaccgcagacggtaagatccaaagagcta | | FALKGAGAGFGVIT | |
| | | acgataagcaaaattccgacttgttctttgccttaaaag | | EFVMRTHPEPGDVV | |
| | | gtgcaggagctggttttggtgtcattactgagttcgtga | | QYSYAITFAKHRDL | |
| | | tgagaacccatccagaacctggtgacgttgttcaatatt | | VPVFKQWQELIFDPT | |
| | | cttacgctatcacttttgctaaacacagagacttggttcc | | LDRRFSSEFVMQEL | |
| | | tgtattcaagcaatggcaagaactgattttcgatccaac | | GVAITATFYGTEDEF | |
| | | acttgatagacgtttctcatctgaatttgtcatgcaagaa | | KKTGIPDRIPKGKVS | |
| | | ttaggtgtcgctataacggccactttttacggcacgga | | VVINNWLGDVAQK | |
| | | ggatgaatttaagaagactggtattccagacagaatcc | | AQDAALWLSDIQSA | |
| | | ccaaaggtaaagtttccgtcgttataaacaattggttgg | | FTSKSLAFTHNDLIS | |
| | | gtgatgtcgcacagaaggctcaagatgcagccttgtg | | EDGIQTMMDYVDSV | |
| | | gcttagtgatattcaatcagctttcacctctaagtccttg | | DRGTLIWFLILDSTG | |
| | | gctttcacccataacgacctaatctcggaagacggtat | | GAINDVPMNATAYR | |
| | | ccaaactatgatggactatgttgattcagtcgatagag | | HRDKVMFFQGYGV | |
| | | gcacattaatttggttcttgattttggattctactggagga | | GIPTLSGKTKDFMSG | |
| | | gctattaatgacgttccaatgaacgctacagcctacag | | VADKIRKASPNELST | |
| | | acacagggacaaagtgatgttcttccaaggttacggtg | | YAGYVDPTLDNAQE | |
| | | ttggtataccaaccctttctggtaagaccaaggattttat | | RYWGPNLPALERIK | |
| | | gtccggtgttgctgataagatccgtaaggcctctccta | | ATWDPKDLFSNPQS | |
| | | acgaattgagcacttacgctggatacgtagacccaact | | VRPNASAKDVEPAA | |
| | | ttggacaatgctcaagaaagatattggggtccaaactt | | SGGSNNSGSKGGDS | |
| | | accagccctagaaagaataaaagctacctgggatcct | | | |
| | | aaggacttattctcaaacccacagtcagtgaggccaa | | | |
| | | acgcttccgccaaggatgtcgaacctgccgcatctgg | | | |
| | | tggttccaataattcgggttctaaaggtggagacagt | | | |
| t807869 | Library | atgggatccggtcatagttctggcttggccacttgctta | 48 | MGSGHSSGLATCLD | 118 |
| | | gatgcagtgtgtaatggtcgtcacgcttgtgtagcttac | | AVCNGRHACVAYP | |
| | | cctgaccacctactgtatcaagcctcttgggtcgatag | | DHLLYQASWVDRY | |
| | | atacaaccttgacatcccagttcatcccatagctgttac | | NLDIPVHPIAVTRPS | |
| | | caggccatcaaacgcagacgatgtcagcggttttgtta | | NADDVSGFVKCAA | |
| | | aatgtgctgccgctaataacgtcagagttcaggctaag | | ANNVRVQAKSGGH | |
| | | tctggtggtcactcgtatgctaattacggcttgggtggt | | SYANYGLGGEDGEL | |
| | | gaggatggtgaattagttattgacttgagacatttgcaa | | VIDLRHLQHFSMDT | |
| | | cacttctcaatggatacgaacacttggcaagctaccatt | | NTWQATIGAGHRL | |
| | | ggtgccggtcacagattatgggacgttacacataagtt | | WDVTHKLHENGKR | |
| | | gcacgaaaacggtaagagagcagtcagccacggaa | | AVSHGTCPGVGIGG | |
| | | cttgcccaggtgttggtattggcggtcatgccaccatc | | HATIGGLGPSSRMW | |
| | | ggtggtctaggtccatcctctcgtatgtggggatcgtgt | | GSCLDHVVEVEVVT | |
| | | ttggatcacgtggtcgaagttgaagttgtgactgctga | | ADGSIRRASERENA | |
| | | cggttctataagaagagcttccgaaagagaaaacgct | | DLFFALKGAGAGFG | |
| | | gatttgttctttgctttaaaaggtgccggtgctggtttcg | | VITEFVMKTHPEPGS | |
| | | gtgtgatcaccgaatttgtaatgaagactcaccctgaa | | VVRYTYSVNFGRHA | |
| | | ccaggatctgttgtcatgaggtacacatactccgttaat | | DMVDVFDQWQALIS | |
| | | ttcggtagacatgcagacatggtcgacgtattcgatca | | DPGLDRRFGSEIIMH | |
| | | atggcaagctttgatttctgatccaggtctggatagaag | | AFGLVISATFHGTRD | |
| | | atttggaagtgaaattatcatgcacgcattcggcctagt | | EYEASGIPDRIPRGN | |
| | | catttccgctacgttccatggtaccagagatgagtatga | | VSVLLDNWLGVVG | |
| | | agcttctggtatcccagacagaatccctcgtggtaacg | | NQAQDAGLWVSEV | |
| | | tgtccgttttgttggacaattggttaggtgtcgttggtaat | | RSSFTSRSLAFRRDQ | |
| | | caggcccaagatgctggattgtgggtttctgaggttag | | LLSRDDIVRMMDFL | |
| | | atcgagtttcacttcacgttcattggcttttagaagggac | | DRTDKGTLVWFLIF | |
| | | caacttctatctcgtgatgatattgtcagaatgatggact | | DVTGGAIGDVRTDA | |
| | | ttttggacagaactgataagggtacgttagtctggttttt | | TAYAHRDKIMFCQG | |
| | | gattttcgacgtcacaggtggtgctattggcgacgtta | | YAVGIPALTRKTRVF | |
| | | gaactgacgcaaccgcctacgctcatagagataagat | | MDGLISTIRETANST | |
| | | catgttctgtcaaggttacgcagttggtataccagctctt | | LTTYPGYVDPSLHD | |
| | | accagaaaaactcgtgtcttcatggacggtttaatttcc | | AQASYWGPNLPRLT | |
| | | actatcagggaaaccgccaactctactctaaccaccta | | EVKTKWDPQDVFH | |
| | | tcccggatacgtcgatccaagtttgcacgacgctcaa | | NPQSVRPSGKD | |
| | | gcttcctactggggtcctaacttgccaagattaacaga | | | |
| | | agttaagactaagtgggatccacaggatgtttttcacaa | | | |
| | | cccacaatctgtaagaccatctggtaaagat | | | |
| t807873 | Library | atgggtaacactacatcaatagctgccggccgtgact | 50 | MGNTTSIAAGRDCL | 120 |
| | | gcctattgagcgctgtgggtggaaatcacgcacatgtt | | LSAVGGNHAHVAFQ | |
| | | gcttttcaggatcaacttttataccaagctaccgccgtc | | DQLLYQATAVEPYN | |
| | | gaaccctataacttgaatatccctgtaactccagcagct | | LNIPVTPAAVTYPQS | |
| | | gttacgtacccacaaagtgctgatgaggttgccgctgt | | ADEVAAVVKCAAD | |
| | | cgttaaatgtgccgctgactacggttataaggttcaag | | YGYKVQARSGGHSF | |
| | | ctaggtccggtggtcactcgttcggtaactacggtttg | | GNYGLGGEDGAIVV | |
| | | ggaggtgaagacggtgctattgtcgttgatatgaagca | | DMKHFDQFSMDEST | |
| | | tttcgatcagttttccatggacgaatctacctatactgca | | YTATIGPGITLGDLD | |
| | | acgatcggtccaggcattactttaggtgatctggatac | | TALYNAGHRAMAH | |
| | | cgccttgtacaacgctggtcacagagctatggctcatg | | GICPTIRTGGHLTIGG | |
| | | gtatctgtccaacaattagaactggtggtcaccttacca | | LGPTARQWGLALDH | |
| | | ttggtggattaggtcctacagctagacaatggggcttg | | VEEVEVVLANSSIVR | |
| | | gccctggaccacgttgaagaagtggaagtcgtcttgg | | ASDTQNQEILFAVK | |
| | | ctaactcgtctatagttagagcatctgacacccaaaatc | | GAAASFGIVTEFKVR | |
| | | aagaaatcttgttcgctgtaaaaggtgctgctgcctcat | | TEEAPGLAVQYSFTF | |
| | | tcggtattgtgactgaatttaaggttcgtactgaggaag | | NLGTAAEKAKLVKD | |
| | | ccccaggtttggccgtccaatattctttcacctttaattta | | WQAFIAQEDLTWKF | |
| | | ggtactgctgctgaaaaggcaaagctggttaaagact | | YSNMNIIDGQIILEGI | |
| | | ggcaagctttcatcgctcaggaggatcttacttggaag | | YFGSKAEYDALGLE | |
| | | ttctactctaacatgaacattattgatggtcaaatcatctt | | EKFPTSEPGTVLVLT | |
| | | ggaaggcatctactttggttctaaggccgaatatgacg | | DWLGMVGHGLEDV | |
| | | ctctaggtttggaggaaaaatttccaacctccgaacca | | ILRLVGNAPTWFYA | |
| | | ggaaccgtcttggtattgactgactggctaggcatggt | | KSLGFAPRALIPDSAI | |
| | | gggtcacggtttggaagatgttatattaagattggtcgg | | DDFFEYIHKNNPGT | |
| | | taatgccccaacttggttctacgccaagtcccttggattt | | VSWFVTLSLEGGAI | |
| | | gcaccaagagcactaattcctgattccgcaattgatga | | NKVPEDATAYGHRD | |
| | | cttcttcgaatacatccataagaacaaccccggtaccg | | VLFWVQIFMINPLGP | |
| | | tttcttggttcgttactttgagtttagagggtggtgctata | | VSQTIYDFADGLYD | |
| | | aataaggtcccagaagatgctaccgcttatggtcatag | | VLAKAVPESAGHAY | |
| | | agatgttctattctgggtacaaattttcatgatcaatccttt | | LGCPDPRMPNAQQA | |
| | | gggtccagtctcacaaactatttacgactttgcagacg | | YWRNNLPRLEELKG | |
| | | gattgtacgatgttttagccaaagctgttccagaaagc | | DLDPKDIFHNPQGV | |
| | | gctggtcatgcttacttgggttgtcccgacccaagaat | | MVVS | |
| | | gccaaacgctcaacaagcttactggaggaacaatttg | | | |
| | | cctagattagaagaacttaagggtgatttggacccaaa | | | |
| | | agatatattccacaacccacaaggtgtcatggttgtttc | | | |
| | | c | | | |
| t807878 | Library | atgggtcaatcccccagttcacttttagccacttgccta | 51 | MGQSPSSLLATCLN | 121 |
| | | aataccgtttgtgacggcagaacagattgtgtagcata | | TVCDGRTDCVAYPN | |
| | | ccctaacaacccattgtatcagatcagctgggtcaacc | | NPLYQISWVNRYNL | |
| | | gttacaatctggatttgccagttactcctattgctgtcac | | DLPVTPIAVTRPQTV | |
| | | cagaccacaaacggttcaagacgtgtctgcttttgttaa | | QDVSAFVKCAATNN | |
| | | atgtgctgccactaacaatataaaggtccaaccaaagt | | IKVQPKSGGHSYAN | |
| | | ctggtggacactcttacgctaactatggtggtgaagac | | YGGEDGALVIDLLK | |
| | | ggtgctttagttattgatttgttgaagttgcaagatttctc | | LQDFSMDAKTWQA | |
| | | catggacgccaaaacctggcaggctactatcggtggt | | TIGGGTKLADVTKR | |
| | | ggtacaaagttggctgatgtcaccaagagactgcatg | | LHDNGKRAISHGTC | |
| | | ataacggtaaaagggcaatttctcacggtacttgtcca | | PGVGIGGHATIGGLG | |
| | | ggcgttggtatcggtggtcatgctaccatcggtggctt | | PTSRMWGSCLDHVV | |
| | | gggacctacttcgagaatgtggggttcctgcttagacc | | EAEVVTADGSIKRA | |
| | | acgtcgtggaggctgaagttgtgactgccgatggtag | | SETENRDLFFALKG | |
| | | tattaagagagcctctgaaacagaaaatcgtgacttgtt | | AGAGFGVVTKFVM | |
| | | cttcgctcttaaaggtgcaggagcaggttttggtgttgt | | KTHPEPGSMVQYSY | |
| | | cacgaagtttgttatgaagacccacccagaaccaggt | | SLSFGKHTDMVPVF | |
| | | agcatggtacaatactcctattcactatctttcggtaaac | | KQWQDLVSDPNLD | |
| | | atactgatatggtaccagtttttaagcaatggcaagattt | | RRFGTEFVAHELGAI | |
| | | agtcagtgaccccaatttggacagaagattcggcact | | ITATFYGTEAEWDA | |
| | | gaatttgttgctcatgagttgggtgctattatcaccgcta | | SGIPQRIPKGKISVIID | |
| | | ctttctacggtacagaagctgaatgggatgctagcgg | | DWLAVISQQAEDAA | |
| | | catcccacaaagaatcccaaagggtaagatatccgtc | | LYLSDIHSAFTVRSL | |
| | | attattgatgattggctagccgttatttcccagcaagca | | AFTAEETLSEQTITR | |
| | | gaggacgctgccctatatttgtctgacattcactccgct | | VMKYIDDTNRGTLL | |
| | | ttcaccgtgcgttctttggccttcaccgctgaagaaaca | | WFLIFDATGGAISDI | |
| | | ttgtctgaacaaactatcactagagttatgaagtacatc | | PMNATAYSHRDKIM | |
| | | gacgatacgaacagaggtaccttgttatggtttttaatat | | YCQGYGIGLPVLNQ | |
| | | tcgacgcaacgggtggtgctataagcgatattcccatg | | HTKDFLTGLTDTIQA | |
| | | aatgctactgcctactcccacagggacaagatcatgta | | SMRQNLTTYPGYVD | |
| | | ctgtcaaggctacggtattggtctaccagtcttaaacca | | PSLANPQQSYWGPN | |
| | | acatactaaagatttccttacgggtcttaccgacactat | | LAMLESIKTTYDPN | |
| | | ccaggcttctatgagacaaaacttgactacctacccag | | DLFHNPQSVRPGNK | |
| | | gttatgttgatccttcattggctaatccacaacaatcttat | | KASMTQEF | |
| | | tggggcccaaaccttgcaatgttggaatcaattaagac | | | |
| | | cacgtatgacccaaacgatttgttccataacccacaat | | | |
| | | ccgttagaccaggtaataaaaaggcttctatgactcaa | | | |
| | | gagttt | | | |
| t807881 | Library | atgagtaataacacccttattcaagcttgcctgttagcc | 52 | MSNNTLIQACLLAA | 122 |
| | | gctctaggattcaactctactagagtaaaatttcctgaa | | LGFNSTRVKFPEAGT | |
| | | gcaggtacaatcgacgccgatccatataacttggactt | | IDADPYNLDLPIVPA | |
| | | gcccatagttccagctgctattactgtcccacgttccac | | AITVPRSTAQVANV | |
| | | ggctcaggtggccaatgtcgttaagtgtgctgcatcga | | VKCAASNGYKVQSR | |
| | | acggttacaaggttcaatcaagatctggcggtcacag | | SGGHSYGNHGLGGT | |
| | | ctacggtaatcatggtttgggcggtaccgatggtgcta | | DGAIVVDLKDLKQF | |
| | | tcgttgtggatttaaaagacttaaagcaattctctatgga | | SMDESTYIASIGSGM | |
| | | tgagtccacttacattgcatctatcggttcaggtatgttg | | LLDEVTHKLYDNGK | |
| | | ttggacgaagtcacccacaagttgtatgataacggtaa | | RAMAHGVCPQVGV | |
| | | gagagccatggctcatggagtttgtccacaagtcggt | | GGHFTIGGLGPTAR | |
| | | gtgggtggtcactttacaattggtggtttgggacctact | | QWGSSLDHVEEVEV | |
| | | gctagacagtggggttcctctcttgaccatgttgaaga | | VLANSSIVRASNTQN | |
| | | agttgaggtcgttttggccaattctagtatagttagggct | | QDVLFAIKGAAASF | |
| | | tccaacactcaaaaccaagacgtcctattcgctatcaa | | GIVTEFKVRTEPAPD | |
| | | aggtgctgctgcttctttcggtattgtgaccgaatttaag | | VAVQYTYELILGNIT | |
| | | gttagaaccgaaccagccccagatgtcgcagttcaat | | ERARIMVDWQDFIS | |
| | | acacttatgaattaattttgggtaatatcacagaaagag | | DPDLSRKFASIMIVF | |
| | | ctaggattatggtcgattggcaggacttcatatccgac | | EHGMLLSGDFYGSK | |
| | | cccgatctttctcgtaaatttgcatcgattatgatcgtctt | | KEFDDLGLADRFPIR | |
| | | cgaacacggtatgttgctgtccggcgatttctacggttc | | KPGNVAILTDWLGM | |
| | | gaagaaggaatttgatgacctgggtttggctgacagat | | TGHAVEELALGIVG | |
| | | tcccaattagaaagccaggtaacgttgctattttgactg | | GIPLHFYAKSMAFTR | |
| | | attggttaggaatgacaggtcatgctgttgaggaattg | | DSLMSPSTFEKLFDY | |
| | | gctttaggtatcgtaggtggcatccctttgcacttttatg | | LDNTDKGTLLWLIY | |
| | | ccaagagtatggctttcacccgtgacagtttaatgtctc | | FDLQSGATSDVPNN | |
| | | catctacgttcgaaaaattgtttgattaccttgataacact | | STAYAHRDTLYWLQ | |
| | | gataagggcaccttgctatggctaatttacttcgatttgc | | SYVVNLVGPVSNTT | |
| | | aatcaggcgccacttcagacgtaccaaataattctaca | | TAFLEGINNLIAQDV | |
| | | gcatacgctcacagagacactttgtactggctgcaatc | | PSANTRAYPGYVDP | |
| | | ctacgtagttaacttggtgggtccagtcagtaacacca | | LMPNPQWRYWGSN | |
| | | ccacagcttttcttgaaggtataaataacttgatcgccc | | LPKLEKIKAAIDPND | |
| | | aagacgtaccttccgccaacactagagcttatccaggt | | VFHNPQSVKPRKQA | |
| | | tacgttgatccattaatgcccaacccacaatggcgttat | | P | |
| | | tggggttcgaatttaccaaagttggaaaagataaaagc | | | |
| | | agctattgaccctaatgacgtcttccataacccacaatc | | | |
| | | agtcaagcctagaaaacaagcaccc | | | |
| t807883 | Library | atgggacagagccaagcattgtcagcctttaaaaagg | 53 | MGQSQALSAFKKDL | 123 |
| | | acctagctgctgctcttaataacgatgatgaattgttcg | | AAALNNDDELFALP | |
| | | ctctgccagacgagcctttctacattaaggaccacgtc | | DEPFYIKDHVKRYN | |
| | | aagcgttataacttagatatcgaaaccacacccttagc | | LDIETTPLAVTYPKT | |
| | | cgtaacgtacccaaaaactaccgcacaagtttcttcca | | TAQVSSIVKLAKDN | |
| | | tagtgaagttggctaaggataacaatttgaaagttcaa | | NLKVQAKCGGHSY | |
| | | gctaagtgcggtggtcattcttatgccaatttttgtgatc | | ANFCDPNGGIIVDLK | |
| | | caaacggtggcatcattgtcgaccttaaacacttccaa | | HFQKFEIDENTWRC | |
| | | aagtttgaaattgacgaaaacacttggagatgtagggt | | RVGGGTLLGDLTKR | |
| | | cggtggtggtactttgctaggtgatttgactaagagaat | | MFEPHKRAMAHGT | |
| | | gttcgaaccacataagagagctatggctcacggtacc | | CPTVGIGGHATIGGL | |
| | | tgtccaacagttggtatcggaggtcatgcaaccatcgg | | GPSSRLWGAALDHV | |
| | | tggcttgggtcctagttcgagattatggggtgctgcctt | | EEVEMVVANGDVIR | |
| | | agaccacgtagaggaagtcgaaatggttgttgctaac | | ANENENSDIFWAVK | |
| | | ggtgacgttattagagccaacgaaaacgaaaattccg | | GAGASFGVITEFVVR | |
| | | atatattctgggctgtcaaaggcgctggtgcctcttttg | | TEPAPGRLVQYSYSF | |
| | | gtgttattactgaatttgttgtaagaactgagccagctcc | | TTGSWKDMAKTFK | |
| | | aggtagattggttcaatactcttattccttcacaaccggt | | AWQTYVSQPDLTRS | |
| | | tcatggaaggacatggccaagactttcaaggcttggc | | FASTATITELGLTISV | |
| | | aaacctacgtcagccaacctgatttgacgcgtagtttc | | TYFGTDEEFDKINFA | |
| | | gcaagtactgctacgatcaccgaactgggtttgactatt | | KNFPGNQTPKTIVFD | |
| | | tctgtcacatacttcggcactgacgaagaatttgataaa | | DYLGAVGHWAEDV | |
| | | ataaatttcgctaaaaatttcccaggtaaccagacccc | | ALEIISPLPAHSYTKT | |
| | | aaagaccatcgtttttgatgattacttgggtgctgtggg | | LTFNHCNQIPDSVID | |
| | | acattgggccgaagatgttgctttagaaattatctctcct | | RMFKYFEEVSKGTL | |
| | | ttgcccgcccactcctatacaaagactttgacttttaacc | | VWFAIFDLAGGRVN | |
| | | actgcaaccaaattccagactctgtgattgatagaatgt | | DIPQDATAYAHRDA | |
| | | tcaaatacttcgaggaagtttcgaagggtacgttagttt | | LFYLQSYAVNPFGP | |
| | | ggtttgccatcttcgatttggctggtggtagagtcaatg | | VSNKSKQFLQGLNK | |
| | | acatcccacaagacgctaccgcatatgctcatagaga | | VIRDGMAEAGENTD | |
| | | tgctttgttctacttacaatcctacgctgtgaacccatttg | | LGAYAGYVDLELGA | |
| | | gtcccgtttctaataaaagtaagcaatttctgcaaggcc | | GAQKAYWRTNLPR | |
| | | ttaacaaggtcatccgtgatggaatggctgaagctggt | | LESIKLKWDPEDVF | |
| | | gaaaatacagacttgggtgcatatgccggctacgttga | | HNPQSVRPGGNDVI | |
| | | tctggaattaggtgctggtgctcagaaggcctactgga | | STPKVVYKKAGFLA | |
| | | gaactaacttgccacgtttggagtctattaagctaaagt | | RLKGCFR | |
| | | gggacccagaggatgtattccacaatcctcaatccgtc | | | |
| | | agaccaggtggtaacgacgttatttctaccccaaaggt | | | |
| | | agtctacaaaaaggctggtttcctagctaggttaaaag | | | |
| | | gttgtttcaga | | | |
| t807917 | Library | atgggtaatacaaccagcattgctggaagggattgcct | 54 | MGNTTSIAGRDCLV | 124 |
| | | agtctctgcattgggcggtaacgccgacttagttgcttt | | SALGGNADLVAFQN | |
| | | tcaaaaccagttgctttaccaaactactgctgtgcacga | | QLLYQTTAVHEYNL | |
| | | gtataatctgaacatacccgttacgcctgccgctatcac | | NIPVTPAAITYPETA | |
| | | ctacccagaaactgctgaacaaattgctgctgtcgtta | | EQIAAVVKCASEYD | |
| | | aatgtgcctccgaatacgattataaggtacaagccaga | | YKVQARSGGHSFGN | |
| | | tcaggtggtcattctttcggtaattacggtttgggtggaa | | YGLGGTDGAVVVD | |
| | | ccgacggtgctgttgtcgttgatatgaagcacttcaac | | MKHFNQFSMDDQT | |
| | | caatttagtatggacgatcaaacttatgaagctgttttag | | YEAVLGPGTTLGDV | |
| | | gtccaggtactaccttgggcgacgtcgatacagaattg | | DTELYNNGKRAMA | |
| | | tacaacaacggtaagcgtgctatggcacatggtatctg | | HGICPTISTGGHFTM | |
| | | tccaacgatttcaaccggtggtcacttcactatgggtg | | GGLGPTARQWGLAL | |
| | | gcttgggtccaaccgccagacaatggggtttagctctt | | DHVEEVEVILANSSI | |
| | | gaccatgtcgaagaagtcgaggttatccttgctaattct | | VRASNTQNQEVFFA | |
| | | tccatcgtaagagcctcgaacacccagaatcaagaag | | VKGAAASFGIVTEF | |
| | | ttttctttgcagttaaaggagctgccgctagtttcggtatt | | KVRTQPAPGLAVQY | |
| | | gtcacagagtttaaggtcagaactcaaccagcacctg | | SYTFNLGSSAKKAQ | |
| | | gtttggctgttcagtattcttacaccttcaacttgggttctt | | FVKDWQSFISAKNL | |
| | | ccgctaagaaagctcaattcgttaaggattggcaaag | | TRQFYTNMVIFDGDI | |
| | | ctttatatccgctaaaaatctaactagacaattttacacta | | ILEGLFFGSKEQYEA | |
| | | acatggtaatcttcgacggtgatattattttggaaggctt | | LGLEERFVPKNPGNI | |
| | | attcttcggctctaaggaacaatacgaagcactgggttt | | LVLTDWLGMVGHA | |
| | | ggaagaacgttttgttccaaagaatccaggtaacatctt | | LEDTILRLVGNTPTW | |
| | | ggttctaacagactggttgggtatggtgggtcacgcct | | FYAKSLGFTPDTLIP | |
| | | tggaagacactatattgagacttgtcggtaacactccta | | SAGIDEFFEYIENNK | |
| | | cctggttttacgcaaagagcttgggtttcactccagata | | AGTSTWFVTLSLEG | |
| | | cgttaattccttctgctggtattgatgaatttttcgaatata | | GAINDVPADATAYG | |
| | | tcgaaaacaacaaggctggcacatccacctggtttgtc | | HRDVLFWVQIFMVS | |
| | | accttatctttagaaggtggtgccattaatgacgtacca | | PTGPVSSTTYDFADG | |
| | | gctgatgctacggcatacggtcacagagatgtgttgtt | | LYNVLTKAVPESEG | |
| | | ctgggttcagattttcatggtcagtccaactggaccagt | | HAYLGCPDPKMAN | |
| | | ttcgtctaccacttatgacttcgctgatggtctgtacaac | | AQQKYWRQNLPRL | |
| | | gtcttgaccaaagctgtgccagagagtgagggtcatg | | EELKAILDPKDTFHN | |
| | | cttacttgggttgtcccgatccaaaaatggccaatgctc | | PQGILPA | |
| | | aacaaaagtattggagacaaaaccttcctagactgga | | | |
| | | agaattgaaggctatcttagatccaaaggacacttttca | | | |
| | | taacccacaaggaattttacccgcc | | | |
| t807918 | Library | atgggtaataccacatccatcgccgctggacgtgattg | 55 | MGNTTSIAAGRDCL | 125 |
| | | cctattgtcggctgttggcggtaaccacgcacatgtcg | | LSAVGGNHAHVAFQ | |
| | | ccttccaggaccaattattgtatcaagctactgctgtgg | | DQLLYQATAVEPYN | |
| | | agccatacaaccttaacatacctgttactccagctgctg | | LNIPVTPAAVTYPQS | |
| | | tcacgtacccccaaagcgcagacgaaattgccgctgt | | ADEIAAVVKCAAEY | |
| | | agttaagtgtgctgctgaatacggttataaagtccaag | | GYKVQARSGGHSFG | |
| | | caagatcaggtggtcactcttttggcaattacggtctgg | | NYGLGGEDGAIVVE | |
| | | gtggtgaagatggtgccattgttgttgaaatgaagcatt | | MKHFNQFSMDESTY | |
| | | tcaaccaattttctatggacgaaagtacctatactgcta | | TATIGPGITLGDLDT | |
| | | ccatcggcccaggtattactcttggtgatttggatacag | | GLYNAGHRAMAHG | |
| | | gtttgtacaacgccggtcacagggcaatggctcatgg | | ICPTIRTGGHLTMGG | |
| | | tatctgtccaactattagaaccggaggtcacttgactat | | LGPTARQWGLALDH | |
| | | gggtggtttaggtccaacagctagacagtggggatta | | VEEVEVVLANSSIVR | |
| | | gctttggaccatgttgaagaggtcgaagtggttttggc | | ASDTQNQDIFFAVK | |
| | | aaattcctctattgtcagagctagcgacacccaaaatc | | GAAASFGIVTEFKVR | |
| | | aagatatattcttcgctgttaagggtgccgctgcctcttt | | TEEAPGLAVQYSFTF | |
| | | tggtatcgtaactgaatttaaagtcagaaccgaagaag | | NLGTAAEKAKLVKD | |
| | | ctcctggattagctgtccaatactccttcactttcaacttg | | WQAFIAQEDLTWKF | |
| | | ggtaccgccgccgaaaaggctaaacttgttaaggact | | YSNMNIFDGQIILEGI | |
| | | ggcaagctttcattgctcaagaggatttgacctggaag | | YFGSKEEYDALGLE | |
| | | ttttactccaacatgaacatcttcgatggtcaaataatctt | | ERFPTSEPGTVLVLT | |
| | | agaaggtatttactttggttctaaggaagaatatgatgc | | DWLGMVGHGLEDV | |
| | | attgggtttagaagagagattcccaacctctgaacctg | | ILRLVGNTPTWFYA | |
| | | gtactgttctggtgttgacagactggttgggtatggttg | | KSLGFAPRALIPDSAI | |
| | | gacacggcctagaggatgtcattttgaggttagtgggt | | DDFFSYIHENNPGTV | |
| | | aatactccaacttggttttatgccaaatcactaggtttcg | | SWFVTLSLEGGAIN | |
| | | ccccacgtgccttgatcccagacagtgctattgatgatt | | KVPEDATAYGHRDV | |
| | | tcttttcttatatacacgaaaacaacccaggtactgtttct | | LFWVQIFMINPLGPV | |
| | | tggttcgtaacgcttagcttggaaggtggcgctatcaa | | SQTTYGFADGLYDV | |
| | | caaggttcccgaagacgctaccgcttacggtcacaga | | LAKAVPESAGHAYL | |
| | | gatgtgttgttttgggtacaaattttcatgattaatccttta | | GCPDPRMPNAQQAY | |
| | | ggtccagtttcgcagactacctacggtttcgcagacgg | | WRSNLPRLEELKGE | |
| | | attgtacgacgtcctagctaaggctgtcccagaatcag | | LDPKDIFHNPQGVM | |
| | | ctggtcatgcatacctgggttgtcccgacccacgtatg | | VVS | |
| | | ccaaacgcccaacaagcttattggagatccaacttgc | | | |
| | | caagattggaagaattaaaaggtgaattggatccaaa | | | |
| | | ggatatctttcataatccacagggtgttatggttgtttct | | | |
| t807926 | Library | atgggaaataccactagcattgcaggtcgtgactgcct | 56 | MGNTTSIAGRDCLIS | 126 |
| | | aatatctgccttaggtggtaactcagctcttgctgctttc | | ALGGNSALAAFPNQ | |
| | | cctaaccaactgttgtggacggccgatgtacacgaat | | LLWTADVHEYNLNL | |
| | | ataatttaaacttgccagttacaccagctgctatcactta | | PVTPAAITYPETAEQ | |
| | | ccccgagactgccgaacagattgcaggcatcgtcaa | | IAGIVKCASDYDYK | |
| | | gtgtgcttccgactacgattacaaagtgcaagctaggt | | VQARSGGHSFGNYG | |
| | | ctggtggtcatagttttggtaattatggtttgggcggaa | | LGGTDGAVVVDMK | |
| | | ccgacggtgccgtcgttgttgatatgaagcacttcaac | | HFNQFSMDDQTYEA | |
| | | caattttcaatggacgatcaaacctacgaagctgttatt | | VIGPGTTLNDVDIEL | |
| | | ggtccaggtacaactttgaacgatgttgatatagaatta | | YNNGKRAMAHGVC | |
| | | tacaataacggtaagagagccatggctcatggcgtct | | PTIKTGGHFTIGGLG | |
| | | gtcctactatcaaaaccggaggtcacttcactattggtg | | PTARQWGLALDHVE | |
| | | gtttgggtccaaccgctagacaatggggtcttgctttg | | EVEVVLANSSIVRAS | |
| | | gaccacgtagaagaggtcgaagtcgttttggctaactc | | NTQNQDVLFAVKG | |
| | | ttccatcgttagagcaagtaatacccaaaaccaagatg | | AAADFGIVTEFKVR | |
| | | tcttgttcgccgttaagggtgctgccgctgactttggaa | | TEPAPGLAVQYSYT | |
| | | ttgtaaccgaatttaaggttagaactgaaccagctcca | | FNLGSTAEKAQFVK | |
| | | ggtttggccgttcagtattcgtatacgttcaacctaggtt | | DWQSFISAKNLTRQ | |
| | | ctactgctgaaaaagctcaattcgtgaaggactggca | | FYNNMVIFDGDIILE | |
| | | atctttcatttccgctaaaaatttaaccagacaattttaca | | GLFFGSKEQYDALG | |
| | | acaatatggtcatcttcgatggtgatatcattctggagg | | LEDHFAPKNPGNILV | |
| | | gtttgttctttggtagcaaggaacaatacgatgccctag | | LTDWLGMVGHALE | |
| | | gtttggaagaccatttcgcacccaagaacccaggtaa | | DTILKLVGNTPTWF | |
| | | catcctggttttaaccgactggcttggcatggtcggcc | | YAKSLGFRQDTLIPS | |
| | | acgctttggaagatacaatacttaagttggtcggtaata | | AGIDEFFEYIDNHTA | |
| | | ctccaacttggttttatgccaagtctttgggtttcagaca | | GTPAWFVTLSLEGG | |
| | | agatactttgattccttccgctggtattgatgaatttttcg | | AINDVAEDATAYAH | |
| | | aatacatagacaaccacacggctggtactccagcttg | | RDVLFWVQLFMVN | |
| | | gttcgttacattatcattggagggtggtgccatcaatga | | PLGPISETTYEFTDG | |
| | | cgtggccgaagatgctactgcatacgcccatcgtgat | | LYDVLARAVPESVG | |
| | | gttttattctgggttcagttgtttatggtcaacccacttgg | | HAYLGCPDPRMENA | |
| | | tccaatctctgaaacaacctacgaatttacggatggttt | | PQKYWRTNLPRLQE | |
| | | gtatgacgttctagctagagctgttcctgagtctgttggt | | LKEELDPKNTFHHP | |
| | | catgcctacttgggatgtccagatccacgtatggaaaa | | QGVIPA | |
| | | cgcacctcagaagtactggagaactaatttacctagat | | | |
| | | tgcaagaactgaaggaagaattggacccaaaaaata | | | |
| | | cattccaccatccacaaggtgttattccagct | | | |
| t807928 | Library | atgggtaataccacatctattgccggcagagactgcct | 57 | MGNTTSIAGRDCLIS | 127 |
| | | aatcagcgctttaggtggagattccgcactggctgtctt | | ALGGDSALAVFPNQ | |
| | | cccaaaccagcttttgtggactgctgatgtgcacgaat | | LLWTADVHEYNLNL | |
| | | acaacttaaatcttcctgtaactccagccgctataacct | | PVTPAAITYPETAEQ | |
| | | atcccgagacagctgaacaaattgccggtatcgttaaa | | IAGIVKCASDYDYK | |
| | | tgtgcttcagactacgattataaggttcaagcacgtagt | | VQARSGGHSFGNYG | |
| | | ggtggtcattcctttggcaactacggtttgggtggtact | | LGGTDGAVVVDMK | |
| | | gacggtgctgttgtcgtcgacatgaagcacttcaatca | | HFNQFSMDDQTYEA | |
| | | attttctatggatgatcaaacctacgaagcagttattggt | | VIGPGTTLNDVDIEL | |
| | | ccaggtactaccttgaacgacgttgacatcgaattgta | | YNNGKRAMAHGVC | |
| | | caacaatggaaagagagctatggctcatggtgtatgtc | | PTIKTGGHFTIGGLG | |
| | | caaccataaaaactggtggtcatttcacgattggtggtt | | PTARQWGLALDHVE | |
| | | tgggtcctacggccagacaatggggcttggctttagat | | EVEVVLANSSIVRAS | |
| | | cacgttgaagaagttgaggtcgtcttggccaactcttc | | NTQNQDVFFAVKGA | |
| | | gatcgtcagggcttctaatactcaaaaccaagatgtctt | | AADFGIVTEFKVRTE | |
| | | tttcgctgttaagggcgccgcagctgacttcggtattgt | | PAPGLAVQYSYTFN | |
| | | gactgaatttaaggttagaacagaaccagctccagga | | LGSTAEKAQFVKDW | |
| | | ttggccgtgcagtatagctatactttcaaccttggtagta | | QSFISAKNLTRQFYN | |
| | | ccgctgaaaaagctcaattcgttaaggattggcaaag | | NMVIFDGDIILEGLF | |
| | | ctttatctccgccaagaacttgacgagacaattctacaa | | FGSKEQYDALGLED | |
| | | taatatggtcattttcgacggtgatattatcttagagggtt | | HFAPKNPGNILVLTD | |
| | | tgttctttggttcgaaggaacaatacgacgctttgggttt | | WLGMVGHALEDTIL | |
| | | ggaagaccactttgcaccaaaaaacccaggtaacatt | | KLVGNTPTWFYAKS | |
| | | ctagttctaaccgattggttaggtatggtaggacacgct | | LGFRQDTLIPSAGID | |
| | | ttagaagatactatcttgaagctagttggtaataccccc | | EFFEYIANHTTGTPV | |
| | | acttggttctatgcaaagtctttgggttttagacaggaca | | WLVTLSLEGGAIND | |
| | | cactgatcccttctgctggaattgatgaatttttcgaata | | VAEDATAYAHRDV | |
| | | cattgctaaccacaccaccggtactcctgtttggctggt | | LFWVQLFMVNPLGP | |
| | | tactttgtcattagaaggtggtgccattaatgatgtagct | | ISETTYEFTDGLYDV | |
| | | gaggatgcaacagcttacgctcatagagatgtcctattt | | LARAVPESVGHAYL | |
| | | tgggttcaattgttcatggttaacccattgggtcctatttc | | GCPDPRMEDAPQKY | |
| | | tgaaacaacttatgaatttacagacggattgtacgacgt | | WRTNLPRLQELKEE | |
| | | cttggcccgtgctgtcccagagtccgtcggtcatgcct | | LDPKNTFHHPQGVIP | |
| | | acttaggctgtccagacccaagaatggaagatgctcc | | A | |
| | | acaaaagtactggcgtaccaacttgccaagattgcaa | | | |
| | | gaattgaaggaagaattagacccaaaaaacacgttcc | | | |
| | | accatccacaaggtgttatacccgcc | | | |
| t807929 | Library | atgggtaataaagcaagtaccacaacgataatcacca | 58 | MGNKASTTTIITTAV | 128 |
| | | ctgctgtacacaagtgccttctgtcggccgtgaacggc | | HKCLLSAVNGNSAQ | |
| | | aactcagctcaggtttccgtccaaaacgacttattgtac | | VSVQNDLLYGVTAV | |
| | | ggtgttaccgctgttcatgaatataatttgaactttccaat | | HEYNLNFPMTPAAV | |
| | | gactcccgctgccgtcactttccctgagacttccgaac | | TFPETSEQVAALVK | |
| | | aagttgctgcattggtcaagtgtgctgccgaatacaag | | CAAEYKYKVQARS | |
| | | tataaagtgcaagctaggagcggaggtcactctttcg | | GGHSFGNHGLGGAD | |
| | | gtaaccatggtctaggtggtgctgatggagctattgttg | | GAIVVDMKHFQQFS | |
| | | tcgatatgaagcactttcaacaattctctatggacaatg | | MDNETHVATIGPGL | |
| | | aaacccacgttgccacaattggcccaggtttgagtcta | | SLGDIDTLLYNAGG | |
| | | ggtgacatcgatacacttttgtacaacgctggtggtag | | RAMSHGICPEIRAGG | |
| | | agccatgagccatggtatttgtccagaaatacgtgccg | | HLTIGGLGLTSRQW | |
| | | gaggtcacttaactatcggtggtttgggtttgacttctcg | | GMSLDHIEEVEVVL | |
| | | tcaatggggtatgtctttagaccatatcgaagaagtcg | | PNSSIVRASETENAD | |
| | | aggtagttttgccaaattcctcgatcgttagagcttctga | | LLFAVKGAAASFGV | |
| | | aaccgaaaatgctgatctattattcgctgttaagggcgc | | VTEFKVRTQLAPKE | |
| | | agctgcatcttttggtgttgtcactgaatttaaggtaaga | | AIQYSYSFKLGSAAQ | |
| | | acgcaacttgcacctaaagaagctattcagtactcata | | RARLFADWQDLALR | |
| | | cagtttcaaattgggttccgctgcccaaagagctagatt | | RDLSRKFTSDFICLQ | |
| | | gttcgctgattggcaagacttggcattaaggagagattt | | DSVIVKGVFFGSKKE | |
| | | gtctcgtaagttcacatccgatttcatttgtttgcaagact | | YNALRIEHHLPGSDS | |
| | | ctgtcattgtgaagggtgtgtttttcggttccaaaaagg | | SKVLVLDDWLGIVT | |
| | | aatataacgccctaagaattgaacatcacttaccaggc | | HVVDDLAVRLGGS | |
| | | tctgacagttctaaggttttggtcttagatgactggttgg | | MSTYFYAKSLGFTR | |
| | | gtattgttacccacgttgtcgatgatctggctgttagatt | | DTLMPPSTITSLFTY | |
| | | aggtggttccatgtcaacttacttttatgccaagtcactt | | LDKAKKGTITWFVT | |
| | | ggttttaccagagatactttgatgccaccatcgacgatc | | FSLVGGAINDYPKN | |
| | | acctctttattcacttacttggacaaagctaagaaaggc | | ATAYPHRDVIYWM | |
| | | acaataacttggttcgtcaccttcagcttggtcggtggt | | QSFAINALGPVLNST | |
| | | gctatcaatgattaccctaagaacgccacggcttatcc | | YDFLDGINELVARD | |
| | | acacagagatgttatctactggatgcaatcttttgctatt | | LPGCAGHAYLGCPD | |
| | | aacgctctgggtcctgttttgaactccacttacgacttct | | PRMEGAERAYWGS | |
| | | tggacggcatcaatgagctagtcgcacgtgatttacca | | NLGRLEDMKGVFDP | |
| | | ggttgtgccggacacgcttatttaggttgcccagatcc | | VDVFWNPQGVGVP | |
| | | cagaatggagggtgctgaaagagcctattggggttca | | VA | |
| | | aacttaggtagacttgaagacatgaaaggtgtctttga | | | |
| | | cccagttgacgttttctggaatccacaaggcgtcggtg | | | |
| | | tccctgttgct | | | |
| t807930 | Library | atgggtaacaccacttccatagcaggccgtgattgcct | 59 | MGNTTSIAGRDCLIS | 129 |
| | | aattagcgctcttggtggtaatagtgctctggccgtgtt | | ALGGNSALAVFPNQ | |
| | | ccccaaccagttattgtggacagctgacgtccatgaat | | LLWTADVHEYNLNL | |
| | | acaatttgaacttacctgttactccagcagctatcacgt | | PVTPAAITYPETAEQ | |
| | | atccagagactgctgaacaaatcgctggaattgttaaa | | IAGIVKCASDYDYK | |
| | | tgtgcctctgattacgactataaggttcaagctaggtct | | VQARSGGHSFGNYG | |
| | | ggtggtcactcctttggtaactacggtttgggcggtac | | LGGTDGAVVVDMK | |
| | | cgacggtgccgtcgtagtcgatatgaagcacttcactc | | HFTQFSMDDQTYEA | |
| | | aattttctatggatgaccaaacctacgaagcagttatag | | VIGPGTTLNDVDIEL | |
| | | gtccaggaacaactttgaatgacgttgatattgaattgt | | YNNGKRAMAHGVC | |
| | | ataacaacggtaaaagagctatggctcatggtgtttgt | | PTIKTGGHFTIGGLG | |
| | | ccaaccatcaagacaggtggtcacttcactattggtgg | | PTARQWGLALDHVE | |
| | | tttaggtccaaccgccagacaatggggattggctttag | | EVEVVLANSSIVRAS | |
| | | accacgtcgaggaagttgaagtcgttttggctaactca | | NTQNQDVFFAVKGA | |
| | | tcgatcgtcagagccagcaatacccaaaatcaggatg | | AADFGIVTEFKVRTE | |
| | | tctttttcgctgtaaagggtgcagctgccgacttcggca | | PAPGLAVQYSYTFN | |
| | | tcgttactgaatttaaagttagaaccgaacctgctccag | | LGSTAEKAQFVKDW | |
| | | gtttggccgtgcaatactcgtatacattcaacctaggtt | | QSFISAKNLTRQFYN | |
| | | ccacggctgagaaggctcaattcgtcaaggattggca | | NMVIFDGDIILEGLF | |
| | | atcttttattagtgcaaagaacttgactagacaattctac | | FGSKEQYDALGLED | |
| | | aacaacatggttattttcgacggtgatattatcttggaag | | HFAPKNPGNILVLTD | |
| | | gtttgttctttggctcaaaagaacagtacgatgctcttgg | | WLGMVGHALEDTIL | |
| | | tttggaagatcatttcgctccaaagaatccaggcaaca | | KLVGNTPTWFYAKS | |
| | | tcttagttttgactgactggctgggtatggtgggtcacg | | LGFRQDTLIPSAGID | |
| | | ctctggaagatacgattttgaagcttgtcggtaataccc | | EFFEYIDNHTAGTPA | |
| | | ccacctggttctatgctaagtctctaggttttagacaag | | WFVTLSLEGGAIND | |
| | | ataccctgattcctagtgctggcatcgatgagttctttga | | VAEDATAYAHRDV | |
| | | atacatcgacaatcacactgccggaactccagcttggt | | LFWVQLFMVNPLGP | |
| | | tcgtaactttatccttggaaggaggtgccataaatgatg | | ISETTYEFTDGLYDV | |
| | | ttgccgaagacgctactgcctatgctcatagagatgttt | | LARAVPESVGHAYL | |
| | | tattttgggttcaattgtttatggtcaaccctttgggtcca | | GCPDPRMENAPQKY | |
| | | atatctgaaactacatacgaatttactgatggtttatacg | | WRTNLPRLQELKEE | |
| | | acgtattggccagagcagtaccagaatccgttggtcat | | LDPKNTFHHPQGVIP | |
| | | gcttaccttggttgtccagacccacgtatggaaaatgc | | A | |
| | | acctcaaaagtactggaggactaacttgcccagacttc | | | |
| | | aggaattgaaagaagagctagacccaaagaacacct | | | |
| | | tccaccatccacaaggtgtcattccagct | | | |
| t807933 | Library | atgggcaacaccacatctatcgctggtagggactgctt | 60 | MGNTTSIAGRDCLV | 130 |
| | | ggtatcagccctgggtggtaatgctggtcttgttgcattt | | SALGGNAGLVAFQN | |
| | | caaaaccagcctttgtatcaaactactgctgtgcacga | | QPLYQTTAVHEYNL | |
| | | atacaatttaaacataccagttaccccagccgctattac | | NIPVTPAAITYPETA | |
| | | gtacccagagactgctgaacaaattgccgctgtcgtc | | EQIAAVVKCASQYD | |
| | | aagtgtgcatcccaatacgattataaagtccaagctag | | YKVQARSGGHSFGN | |
| | | aagtggaggtcatagcttcggtaattacggtctaggcg | | YGLGGTDGAVVVD | |
| | | gtacagatggtgctgttgttgttgacatgaagtacttca | | MKYFNQFSMDDQT | |
| | | accaattttctatggacgatcagacctacgaagctgtc | | YEAVIGPGTTLGDV | |
| | | atcggtcctggtacaaccttgggagatgtcgacgtcg | | DVELYNNGKRAMA | |
| | | aattatataacaacggtaagcgtgccatggctcacggt | | HGVCPTISTGGHFT | |
| | | gtttgtccaactatttcgactggaggtcatttcactatgg | | MGGLGPTARQWGL | |
| | | gtggtttgggtcccaccgccagacaatggggcttagc | | ALDHVEEVEVVLAN | |
| | | cttggaccacgttgaagaagtagaagtagttttagcaa | | SSIVRASNTQNQEVF | |
| | | actcctctatcgtgagagctagcaatacgcaaaatcaa | | FAVKGAAASFGIVT | |
| | | gaggttttctttgctgttaaaggcgctgctgcctctttcg | | EFKVRTQPAPGIAVQ | |
| | | gtattgtcactgaatttaaggttagaactcagccagctc | | YSYTFNLGSSAEKA | |
| | | caggtatagcagtccaatattcctacaccttcaacttgg | | QFIKDWQSFVSAKN | |
| | | gttcgtctgctgagaaggctcaattcatcaaagattgg | | LTRQFYTNMVIFDG | |
| | | caatcatttgtctccgctaagaacttgaccagacaattc | | DIILEGLFFGSKEQY | |
| | | tacaccaatatggttattttcgatggtgatattatcctaga | | EALRLEERFVPKNPG | |
| | | aggtttgtttttcggttccaaggaacagtatgaagcatt | | NILVLTDWLGMVGH | |
| | | gcgtcttgaagagagatttgtgccaaaaaacccaggt | | ALEDTILRLVGNTPT | |
| | | aacatcttggttctaactgactggctaggtatggtcgga | | WFYAKSLGFTPDTLI | |
| | | catgccttggaagacacaatcttgagacttgttggtaat | | PSSGIDEFFKYIENN | |
| | | actcctacttggttttacgctaagtctctgggtttcacac | | KAGTSTWFVTLSLE | |
| | | cagatacgttgattccatcttctggaattgatgaatttttc | | GGAINDVPADATAY | |
| | | aagtacatagaaaacaataaggccggcacctccactt | | GHRDVLFWVQIFMV | |
| | | ggtttgttacattatcattggaaggcggtgctatcaacg | | SPTGPVSSTTYDFAD | |
| | | atgtacctgctgacgccaccgcttatggtcacagagat | | GLYNVLTKAVPESE | |
| | | gttttattctgggtccaaattttcatggtttcaccaactgg | | GHAYLGCPDPKMA | |
| | | tccagtttcttctaccacctatgacttcgctgatggtttat | | NAQQKYWRQNLPR | |
| | | acaatgtcttgactaaagctgtacccgagagtgaagg | | LEELKETLDPKDTFH | |
| | | ccatgcttacttgggttgtccagaccctaagatggctaa | | NPQGILPA | |
| | | tgcacaacaaaagtactggagacaaaacctaccaag | | | |
| | | acttgaagaattgaaagaaaccctagaccccaaggat | | | |
| | | acttttcacaacccacaaggtatcctaccagcc | | | |
| t807943 | Library | atgaatccctcaattccatcctctagtatgggcaacact | 61 | MNPSIPSSSMGNTTS | 131 |
| | | acctcgatagccggtagagactgtctagtgtctgcact | | IAGRDCLVSALGGN | |
| | | tggaggtaacgctggtttagttgctttccaaaaccagc | | AGLVAFQNQPLYQT | |
| | | cactgtatcaaacaactgctgtacacgaatacaatttga | | TAVHEYNLNTPVTP | |
| | | atacccctgttacgccagccgctatcacttacccagag | | AAITYPETAEHIAAV | |
| | | acagctgaacatattgctgccgtcgttaaatgcgcaag | | VKCASQYDYKVQA | |
| | | ccaatatgattacaaggtccaagctcgttctggtggtca | | RSGGHSFGNYGLGG | |
| | | ctcctttggtaactacggtttgggtggtaccgatggag | | TDGAVVVDMKYFN | |
| | | ctgtcgttgttgacatgaagtatttcaaccaattttctatg | | QFSMDDQTYEAVIG | |
| | | gatgaccaaacctacgaagctgttatcggtcctggtac | | PGTTLGDVDVELYN | |
| | | tactttgggtgatgtggatgtagaattgtataacaacgg | | NGKRAMAHGVCPTI | |
| | | taaaagagccatggctcatggtgtctgtccaactatttc | | STGGHFTMGGLGPT | |
| | | caccggcggtcacttcacaatgggcggtttaggtcca | | ARQWGLALDHVEE | |
| | | actgctagacaatggggtttggctcttgaccacgtcga | | VEVVLANSSIVRASN | |
| | | agaagttgaggtggttctagcaaatagttctatcgtcag | | TQNQEVFFAVKGAA | |
| | | ggcctcgaatactcagaatcaagaagttttctttgcagt | | ASFGIVTEFKVRTQP | |
| | | aaagggagctgctgcttcttttggtatcgttaccgaattt | | APGIAVQYSYTFNL | |
| | | aaggtcagaacgcaaccagctccaggaattgctgttc | | GSSAEKAQFIKDWQ | |
| | | aatactcatacaccttcaacttgggttccagcgccgaa | | SFVSAKNLTRQFYT | |
| | | aaggctcagttcattaaggactggcaatctttcgtgtcc | | NMVIFDGDIILEGLF | |
| | | gctaaaaacttaaccagacaattctacacaaacatggt | | FGSKEQYEALGLEE | |
| | | tatatttgacggtgatattatcttggaaggtctatttttcg | | RFVPKNPGNILVLTD | |
| | | gttccaaagagcaatatgaagctttgggtttggaagaa | | WLGMVGHALEDTIL | |
| | | agattcgtcccaaagaaccctggcaatatcctagtttta | | RLVGNTPTWFYAKS | |
| | | acggattggttgggtatggtcggacatgccttagagg | | LGFTPDTLIPSSGIDE | |
| | | atacaatattgagattggttggtaacactcccacctggt | | FFEYIENNKAGTST | |
| | | tctacgccaagtcccttggttttactccagacacattgat | | WFVTLSLEGGAIND | |
| | | tccttcttctggtatcgatgaatttttcgaatatattgaaaa | | VPADATAYGHRDVL | |
| | | caataaggcaggtacttctacctggtttgtcaccctttca | | FWVQIFMVSPTGPV | |
| | | ttggaaggtggtgccattaacgacgtcccagctgatg | | SSTTYDFADGLYNV | |
| | | ctactgcatacggtcatcgtgacgtgctattctgggttc | | LTKAVPESEGHAYL | |
| | | agatatttatggtaagtcccactggcccagtaagttcca | | GCPDPKMANAQQK | |
| | | cgacttacgatttcgctgacggtttatataatgttctgact | | YWRQNLPRLEELKE | |
| | | aaagctgtgccagaatctgagggtcacgcctacttag | | TLDPKDTFHNPQGIL | |
| | | gatgtccagatccaaagatggctaatgcacaacaaaa | | PA | |
| | | atactggagacaaaacttgccaagattggaagaacta | | | |
| | | aaggaaactttggacccaaaagataccttccataatcc | | | |
| | | tcaaggcatccttcccgcc | | | |
| t807945 | Library | atgggtaatactacctcaatagccggcagagattgcct | 62 | MGNTTSIAGRDCLV | 132 |
| | | agtctccgctttgggaggtaacgcaggtctggtggctt | | SALGGNAGLVAFQN | |
| | | ttcaaaaccagcctttgtatcaaacgacagctgtacac | | QPLYQTTAVHEYNL | |
| | | gaatacaatcttaacattcccgtcactccagccgctatc | | NIPVTPAAITYPETA | |
| | | acctacccagagactgctgaacaaatcgccgcagttg | | EQIAAVVKCASQYD | |
| | | ttaaatgtgcttcgcaatacgactataaggttcaagcta | | YKVQARSGGHSFGN | |
| | | ggtctggtggtcattccttcggtaactacggattaggc | | YGLGGTDGAVVVD | |
| | | ggtacagacggtgccgtcgttgttgatttgaagtacttc | | LKYFNQFSMDDQTY | |
| | | aatcagttttctatggatgaccaaacctatgaagctgtc | | EAVIGPGTTLGDVD | |
| | | attggtccaggtactaccttgggtgatgtagacgttgaa | | VELYNNGKRAMAH | |
| | | ttatataacaacggtaagcgtgctatggcccacggtgt | | GVCPTISTGGHFTM | |
| | | atgtccaactattagcacgggtggtcatttcactatggg | | GGLGPTARQWGLAL | |
| | | tggtcttggacctacggctagacaatggggtttagcctt | | DHVEEVEVVLANSSI | |
| | | ggatcacgtcgaagaagttgaggtcgttttggctaact | | VRASNTQNQEVFFA | |
| | | ctagtatcgttagagctagcaatacccaaaatcaagaa | | VKGAAASFGIVTEF | |
| | | gtgtttttcgctgttaaaggcgcagccgcttcgttcggt | | KVRTQPAPGIAVQY | |
| | | attgtcactgaatttaaggttagaactcaaccagctcca | | SYTFNLGSSAEKAQF | |
| | | ggtattgctgttcaatactcttacaccttcaatttgggctc | | IKDWQSFVSAKNLT | |
| | | ttccgccgagaaggcacagtttataaaagactggcaa | | RQFYTNMVIFDGDII | |
| | | tcattcgtttctgctaagaacttgacaagacaattctata | | LEGLFFGSKEQYEAL | |
| | | ccaacatggtcatctttgacggtgatattatcctagaag | | RLEERFVPKNPGNIL | |
| | | gtctgtttttcggtagtaaggaacaatacgaagctttgc | | VLTDWLGMVGHAL | |
| | | gtttagaagaaagattcgtgcccaagaaccctggtaa | | EDTILRLVGNTPTWF | |
| | | cattttggttttaactgattggctaggtatggtcggtcac | | YAKSLGFTPDTLIPS | |
| | | gctttggaggacacaatcctaagattggttggaaatac | | SGIDEFFEYIENNKA | |
| | | cccaacttggttctacgctaagtccttgggatttactcca | | GTSTWFVTLSLEGG | |
| | | gatactttgataccatcttccggtatcgacgaatttttcg | | AINDVPADATAYGH | |
| | | aatatattgaaaacaataaagccggtacctctacatggt | | RDVLFWVQIFMVSP | |
| | | tcgtaaccctttctcttgagggtggagccatcaacgac | | TGPVSSTTYDFADG | |
| | | gttccagctgatgctactgcatacggtcatagagatgt | | LYNVLTKVVPESEG | |
| | | cttgttttgggtacagattttcatggtcagccctacaggt | | HAYLGCPDPKMAN | |
| | | ccagtttcctctacgacctatgactttgctgatggtttata | | AQQKYWRQNLPRL | |
| | | caacgttttgactaaggtggttccagaatccgaaggcc | | EELKETLDPKDTFH | |
| | | acgcttacttaggttgtccagacccaaaaatggccaat | | NPQGVLITEVGSATD | |
| | | gctcaacaaaagtattggaggcaaaatttgccaagact | | FWNLVEAIILISQLH | |
| | | agaagaactaaaagaaacactggaccctaaggatact | | ESVGQTYNMVPEM | |
| | | tttcacaatccacaaggcgtcttgatcaccgaggttggt | | GEQPVREMTKMFR | |
| | | tccgccacggacttctggaacttagttgaagctattatc | | MLEKTIQVSLEGLPY | |
| | | ttaatctctcagttgcatgaatcagtcggccaaacatac | | EEWLNRLQVENDD | |
| | | aacatggtgcccgagatgggtgaacaacctgttagag | | DPLRPLLPMFEEKV | |
| | | aaatgactaagatgttccgtatgttggaaaagactattc | | YDGRCQWEMYENM | |
| | | aagtcagcttggaaggtcttccatacgaggaatggttg | | PISDTENLRQYLQDV | |
| | | aacagactgcaagtggaaaacgatgatgatccactga | | PELATCPFLDQDIFK | |
| | | ggccactgttgccaatgtttgaagaaaaagtctacgac | | KFLSSLGLA | |
| | | ggtagatgccaatgggaaatgtacgagaacatgccta | | | |
| | | tttcggacaccgaaaacttgagacaatacttgcaagat | | | |
| | | gttcctgaattagcaacttgtccattcttggatcaagata | | | |
| | | tatttaagaagttcctttcctctcttggtttggca | | | |
| t807950 | Library | atgggcaatacaacttcgatagctggtagagactgcct | 64 | MGNTTSIAGRDCLIS | 134 |
| | | tatttcagcactgggtggaaacagcgccttagctgcttt | | ALGGNSALAAFPNE | |
| | | tcccaacgagctattgtggacggccgatgtccatgaat | | LLWTADVHEYNLNL | |
| | | acaatttgaacttgccagtgactcctgctgctatcacct | | PVTPAAITYPETAEQ | |
| | | atccagaaaccgctgaacaaattgcaggagtagttaa | | IAGVVKCASDYDYK | |
| | | atgtgcctctgactacgattacaaggtccaggctcgttc | | VQARSGGHSFGNYG | |
| | | cggtggtcacagtttcggtaactatggtttaggtggtgc | | LGGADGAVVVDMK | |
| | | agatggtgctgttgtcgttgacatgaagcacttcactca | | HFTQFSMDDETYEA | |
| | | attttctatggacgatgaaacctacgaagctgttatcgg | | VIGPGTTLNDVDIEL | |
| | | tccaggcactacattgaatgatgttgacattgaattatat | | YNNGKRAMAHGVC | |
| | | aacaacggtaagagagccatggctcatggtgtgtgtc | | PTIKTGGHFTIGGLG | |
| | | ctaccatcaaaacaggtggtcacttcactattggcggtt | | PTARQWGLALDHVE | |
| | | tgggtccaactgctagacaatggggtttagctttggatc | | EVEVVLANSSIVRAS | |
| | | acgtcgaggaagtcgaagttgttttggccaactcttcc | | NTQNQDVFFAVKGA | |
| | | attgtcagggcatctaatacccaaaaccaagacgtgtt | | AANFGIVTEFKVRTE | |
| | | tttcgctgttaagggcgccgctgctaacttcggaatcgt | | PAPGLAVQYSYTFN | |
| | | taccgaatttaaggtcagaactgaaccagcaccaggtt | | LGSTAEKAQFVKDW | |
| | | tggccgtccagtactcgtatactttcaatttgggtagtac | | QSFISAKNLTRQFYN | |
| | | cgccgaaaaagctcaatttgttaaggactggcaatcttt | | NMVIFDGDIILEGLF | |
| | | catttccgctaagaatcttactagacaattttacaataac | | FGSKEQYDALGLED | |
| | | atggtaatcttcgatggtgatatcattttggaaggtttgtt | | HFAPKNPGNILVLTD | |
| | | ctttggttccaaagaacaatacgatgctctgggtcttga | | WLGMVGHALEDTIL | |
| | | agatcatttcgctccaaagaaccctggtaacatattggt | | KLVGNTPTWFYAKS | |
| | | cctaaccgactggctaggtatggttggtcatgccttag | | LGFRQDTLIPSAGID | |
| | | aagacaccatcttgaagcttgttggtaatacaccaactt | | EFFEYIANHTAGTPA | |
| | | ggttctatgcaaaatctttgggctttcgtcaagatactct | | WFVTLSLEGGAINDI | |
| | | gatcccatcagctggcattgacgaatttttcgagtacat | | AEDATAYAHRDVLF | |
| | | cgctaaccacaccgctggtactccagcctggtttgtaa | | WVQLFMVNPLGPIS | |
| | | cgttgtctttagagggtggtgctattaacgatatcgccg | | DTTYEFTDGLYDVL | |
| | | aagatgctacggcttacgcccatagagatgttctattct | | ARAVPESVGHAYLG | |
| | | gggtccaactgttcatggtcaaccctttgggtccaataa | | CPDPRMEDAQQKY | |
| | | gcgacacaacttacgaatttactgatggattatatgacg | | WRTNLPRLQELKEE | |
| | | tattggcaagagcagttcccgaatccgttggtcacgct | | LDPKNTFHHPQGVM | |
| | | tacttaggttgtccagatccaagaatggaagatgctca | | PA | |
| | | acaaaagtactggagaaccaacctgcctcgtttgcaa | | | |
| | | gagcttaaagaagaattggacccaaagaatactttcca | | | |
| | | tcacccacagggtgtcatgccagct | | | |
| t807955 | Library | atgggtaatacgacatcaatcgcagccggaagagact | 65 | MGNTTSIAAGRDCL | 135 |
| | | gccttctgtcggctgtcggtggcaaccacgctcatgta | | LSAVGGNHAHVAFQ | |
| | | gcctttcaggatcaattattgtaccaagctactgctgtg | | DQLLYQATAVEPYN | |
| | | gagccatataacctaaacatacctgttacccccgccgc | | LNIPVTPAAVTYPQS | |
| | | tgttacttacccacaatctgctgaagaaattgcagctgt | | AEEIAAVVQCASEY | |
| | | cgttcaatgtgcttccgaatatggttacaaggttcaagc | | GYKVQARSGGHSFG | |
| | | tcgtagcggtggtcactccttcggtaattacggtttggg | | NYGLGGEDGAIVVE | |
| | | cggtgaagatggtgccatcgtcgttgaaatgaaacatt | | MKHFNQFSMDESTN | |
| | | tcaatcaattttctatggacgaatctaccaacattgctac | | IATIGPGITLGDLDTA | |
| | | tattggtccaggtatcaccttgggtgacttggatactgc | | LYNAGYRAMAHGIC | |
| | | tttatacaacgccggatatagagcaatggctcacggta | | PTIRTGGHLTMGGL | |
| | | tatgtccaacaatcagaacaggtggacatttgaccatg | | GPTARQWGLALDH | |
| | | ggtggtctaggtcctactgccaggcagtggggcttgg | | VEEVEVVLANSSIVR | |
| | | ccttggatcacgttgaggaagtcgaagttgtgttagcta | | ASDTQNQDIFFAVK | |
| | | actcttccattgttagagcttcagatactcaaaatcaag | | GAAASFGIVTEFKVR | |
| | | acattttcttcgctgtcaagggtgctgccgctagttttgg | | TEQAPGLAVQYSFT | |
| | | tattgttaccgaatttaaggtcagaactgaacaagctcc | | FNLQTPAEKAKLVK | |
| | | aggtcttgccgtacaatattctttcactttcaacttacaga | | DWQAFIAQEDLTWK | |
| | | ccccagcagaaaaagcaaagttggtaaaagactggc | | FYSNMNIFDGQIILE | |
| | | aagctttcatcgcccaagaggatttaacatggaagtttt | | GIYFGSKAEYDALG | |
| | | actcaaatatgaatattttcgatggtcaaatcattctgga | | LEKRFPTSEPGTVLV | |
| | | aggaatctacttcggttccaaggctgaatatgacgctct | | LTDWLGMVGHGLE | |
| | | aggtttggagaagagatttcccacttctgaaccaggta | | DVILRLVGNTPTWF | |
| | | ccgtcttggtcttgacagattggctaggtatggtcggtc | | YAKSLGFTPRALIPD | |
| | | acggcttagaagatgttatattgcgtttagttggtaacac | | SAIDDFFNYIHKNNP | |
| | | cccaacttggttttacgccaaaagtttgggcttcacgcc | | GTVSWFVTLSLEGG | |
| | | aagagctttgatcccagactctgctattgatgactttttc | | AINKVPEDATAYGH | |
| | | aactatatccacaagaataaccctggtactgttagttgg | | RDVLFWVQIFMINPL | |
| | | ttcgttactttgtctcttgaaggtggtgctataaataaagt | | GPVSQTTYGFADGL | |
| | | cccagaagacgctaccgcctacggtcatagagatgta | | YDVLAKAVPESAGH | |
| | | ttgttttgggttcagatatttatgattaacccattaggccc | | AYLGCPDPRMPNAQ | |
| | | cgtcagccaaactacatacggtttcgctgacggtttgta | | QAYWRSNLPRLEEL | |
| | | cgatgttttggctaaggcagttccagagtccgcaggtc | | KGELDPKDVFHNPQ | |
| | | atgcttacttgggctgtcctgacccaaggatgccaaac | | GVMVVS | |
| | | gcccaacaagcatactggagatccaacctacctagat | | | |
| | | tggaagaactgaagggtgaattggatccaaaagacgt | | | |
| | | ttttcataaccctcaaggtgtaatggtcgtcagc | | | |
| t807965 | Library | atgggtaacacgacctctatcgctgccggacgtgact | 66 | MGNTTSIAAGRDCLI | 136 |
| | | gtctgatttcggcagtcggtgctgctaatgttgccttcc | | SAVGAANVAFQDQL | |
| | | aggatcaattattgtaccaagctacagctgtacaacctt | | LYQATAVQPYNLNI | |
| | | ataacctaaacataccagttactccagccgctgttacct | | PVTPAAVTYPQSAD | |
| | | acccacaaagcgcagacgaaattgctgccgtggtca | | EIAAVVKCASEYGY | |
| | | agtgcgcttcagagtatggctacaaagttcaagctagg | | KVQARSGGHSFGNY | |
| | | tccggtggtcactcctttggtaattacggtcttggtggc | | GLGGQDGAIVIEMK | |
| | | caagatggtgccatcgttattgaaatgaagcatttctct | | HFSQFSMDESTFIATI | |
| | | cagttttctatggatgaatcaaccttcatcgctactatag | | GPGITLGDLDTDLY | |
| | | gtcccggtattactttgggtgacttggatactgacttgta | | NAGHRAMAHGICPT | |
| | | caacgccggacacagagctatggctcatggtatctgt | | IRTGGHLTVGGLGPT | |
| | | ccaactattagaacgggtggtcacttaacagtcggtgg | | ARQWGLALDHVEE | |
| | | actaggtcctaccgctagacaatggggtttggcattgg | | VEVVLANSSIVRASD | |
| | | atcacgtagaagaagtcgaggtggttttagccaacag | | TQNQDLFFAIKGAA | |
| | | ctccattgtcagagcttctgacactcaaaatcaagattt | | ASFGIVTEFKVRTEQ | |
| | | gttctttgctattaagggtgcagctgcctctttcggaatc | | APGMAVQYSYTFHL | |
| | | gttaccgaatttaaagtcagaactgaacaagctccagg | | GTSAEKAKFVKDW | |
| | | tatggctgtccaatatagttacactttccatctgggcaca | | QAFIAQENLTWKFY | |
| | | tccgcagaaaaggctaagttcgttaaagattggcagg | | TNLVIFDDQIILEGIY | |
| | | cattcatcgctcaagagaacttaacttggaagttttatac | | FGTKEEYDSLGLEQ | |
| | | caatttggttattttcgacgatcaaatcatactagaaggt | | RFPPTDAGTVLILTD | |
| | | atctactttggtacgaaggaagaatacgatagtttaggt | | WLAMIGHGLEDTIL | |
| | | ttggaacaacgtttcccacctacggacgccggcactg | | KLVGDTPTWFYAKS | |
| | | ttttgattttaaccgattggctagctatgatcggtcatggt | | LGFTPRALIPDSAIDE | |
| | | ttggaggacaccatcttgaagctagtcggtgatacacc | | FFDYIHENNPGTLA | |
| | | aacctggttttacgccaagtctttaggttttaccccaaga | | WFVTLSLEGGAINA | |
| | | gctttgattcccgacagtgctatcgacgaatttttcgatt | | VPEDATAYGHRDVL | |
| | | atatccacgaaaataacccaggaactttggcatggttc | | FWFQLFVINPLGPIS | |
| | | gttactttgtctttggaaggtggtgccattaacgctgtcc | | QTTYGFADGLYDVL | |
| | | ctgaagatgctacagcttacggtcatagagatgtgcttt | | AQAVPESVSHAYMG | |
| | | tttggttccaacttttcgtgattaacccattgggtccaatt | | CPDPRLPNAQYAYW | |
| | | tcccaaaccacatacggatttgctgacggtctttatgat | | RSNLPKLEELKGILD | |
| | | gttttggcccaagctgtcccagaatctgtgagccacgc | | PEDIFHNPQGVVPS | |
| | | atatatgggttgtccagaccctagattgccaaatgccc | | | |
| | | aatacgcttattggcgttccaatttaccaaagttggagg | | | |
| | | aattaaaaggtatattagacccagaagacatctttcaca | | | |
| | | acccacagggtgttgttccttca | | | |
| t807974 | Library | atgttatcaacaatggccttctcttttgttttgagaattttgt | 67 | MLSTMAFSFVLRILS | 137 |
| | | cccctctattcttgatactacagcttagcacggctgcttc | | PLFLILQLSTAASTST | |
| | | gaccagtactttgcgtcaatgcttgctgaccgcagtcc | | LRQCLLTAVQNDPT | |
| | | aaaacgatccaactttagtagctgtggacggtgatttgt | | LVAVDGDLLYQTLA | |
| | | tgtatcaaactttagccgttcaagtttacaatcttaactg | | VQVYNLNWPVTPA | |
| | | gccagtcacacccgctgctgttgcatttccaaaatctac | | AVAFPKSTQQVASIV | |
| | | ccaacaagttgcttctatcgtaaattgtgccgcttcccta | | NCAASLGYKVQAKS | |
| | | ggctacaaggtccaagccaagtctggaggtcactcct | | GGHSYGNYGLGGT | |
| | | acggtaactatggtctgggtggtactaacggtgctatta | | NGAISINLKNMKSFS | |
| | | gcatcaacttaaagaatatgaaatcattctctatgaatta | | MNYTNYQATVGAG | |
| | | caccaactaccaggctacagtcggtgccggtatgttg | | MLNGELDEYLHNA | |
| | | aatggcgaattggacgagtatttacataacgctggtgg | | GGRAVAHGTSPQIG | |
| | | tagggccgttgctcacggaacctctccacaaattggtg | | VGGHATIGGLGPSA | |
| | | tcggtggtcatgctactatcggtggattgggtccatctg | | RQYGMELDHVLEAE | |
| | | caagacaatacggtatggaacttgaccacgttttggaa | | VVLANGTVVRASST | |
| | | gctgaagttgttctggctaacggcacggtagtcagag | | QNSDLLFAIKGAGA | |
| | | caagttcaactcaaaactcagatttgttgttcgccattaa | | SFGVVTEFVFRTEPE | |
| | | gggtgctggtgccagctttggtgttgtcactgagttcgt | | PGSAVQYTFTFGLGS | |
| | | ctttagaacagaacctgaaccaggtagtgctgtgcagt | | TSARADLFKKWQSF | |
| | | ataccttcacttttggtttaggctccacgtctgctagagc | | ISQPDLTRKFASICTL | |
| | | agatttgttcaagaaatggcaatccttcatatcccaacc | | LDHVLVISGTFFGTK | |
| | | agacttgactcgtaagtttgcctctatctgtacgctattg | | EEYDALGLEDQFPG | |
| | | gatcatgtacttgtaattagcggtacctttttcggtactaa | | HTNSTVIVFTDWLG | |
| | | ggaagaatacgacgctttgggacttgaagatcaattcc | | LVAQWAEQSILDLT | |
| | | ccggtcacactaattcgaccgttatcgtgtttaccgatt | | GDIPADFYARCLSFT | |
| | | ggttaggcttggttgctcaatgggctgagcaatctatct | | EKTLIPSNGVDQLFE | |
| | | tggacttgactggcgacattccagctgatttctacgcca | | YLDSADTGALLWFV | |
| | | gatgtctgtcctttaccgaaaaaaccctgattccttctaa | | IFDLEGGAINDVPMD | |
| | | cggtgtcgaccagttattcgaatatttggatagtgcaga | | ATGYAHRDTLFWLQ | |
| | | cactggtgctttattatggttcgtcattttcgacttggaag | | SYAITLGSVSETTYD | |
| | | gtggtgctattaacgatgttccaatggatgctactggtt | | FLDSVNEIIRNNTPG | |
| | | acgcacacagagataccttgttttggctacaatcatac | | LGNGVYPGYVDPRL | |
| | | gctatcacattgggttctgtttccgaaaccacttatgattt | | ENAREAYWGSNLPR | |
| | | cttagattctgttaacgaaatcataagaaataatacccct | | LMQIKSLYDPTDLFH | |
| | | ggtttgggtaatggtgtttaccctggttacgtcgaccca | | NPQGVLPA | |
| | | agattagaaaacgctagagaagcttattggggttctaa | | | |
| | | tttgccacgtttgatgcaaataaagtctttgtatgaccca | | | |
| | | acagacttgtttcataacccacaaggtgtactaccagc | | | |
| | | c | | | |
| t807980 | Library | atgggcaataccacatccattgccggacgtgattgcct | 68 | MGNTTSIAGRDCLIS | 138 |
| | | gatcagtgcattgggtggtaactcggctttagctgtcttt | | ALGGNSALAVFPNE | |
| | | cctaacgaattgctatggacggctgacgtgcatgagta | | LLWTADVHEYNLNL | |
| | | taatttgaaccttcccgttactccagctgccataacttac | | PVTPAAITYPETAAQ | |
| | | ccagaaaccgctgctcagattgcaggagttgtcaagt | | IAGVVKCASDYDYK | |
| | | gtgccagcgattacgactataaagttcaagctagatca | | VQARSGGHSFGNYG | |
| | | ggtggtcactctttcggtaactacggtttaggtggtgca | | LGGADGAVVVDMK | |
| | | gatggagctgtagttgttgacatgaagcacttcactca | | HFTQFSMDDETYEA | |
| | | attttctatggatgacgaaacttacgaagctgtcatcgg | | VIGPGTTLNDVDIEL | |
| | | tccaggtaccacattgaatgacgttgatattgaattgta | | YNNGKRAMAHGVC | |
| | | caacaatggtaaaagggccatggctcatggtgtctgtc | | PTIKTGGHFTIGGLG | |
| | | ctaccatcaagactggtggccacttcaccattggtggtt | | PTARQWGLALDHVE | |
| | | taggcccaactgccagacaatggggtctggctttagat | | EVEVVLANSSIVRAS | |
| | | catgttgaagaggtagaagtcgtgttggctaactcttcc | | NTQNQDVFFAVKGA | |
| | | atagtcagagcctctaatacacaaaaccaagatgtctt | | AANFGIVTEFKVRTE | |
| | | ctttgctgttaagggtgcagctgcaaacttcggtattgtt | | PAPGLAVQYSYTFN | |
| | | accgaatttaaggtgagaactgaaccagctccaggttt | | LGSTAEKAQFVKDW | |
| | | ggctgttcaatattcgtacactttcaatttgggttctaccg | | QSFISAKNLTRQFYN | |
| | | ccgaaaaagctcagttcgtcaaggactggcaatccttt | | NMVIFDGDIILEGLF | |
| | | atctccgcaaagaacttgacgcgtcaattctataataac | | FGSKEQYDALGLED | |
| | | atggttatctttgacggagacattatccttgagggtttgtt | | HFAPKNPGNILVLTD | |
| | | tttcggttcaaaggaacaatacgatgccctaggtttaga | | WLGMVGHALEDTIL | |
| | | agatcacttcgctccaaagaaccccggcaacatcttg | | KLVGNTPTWFYAKS | |
| | | gttcttactgactggttaggtatggtaggtcacgctttgg | | LGFRQDTLIPSAGID | |
| | | aagatactattttgaaactggttggtaacacaccaacat | | EFFEYIANHTAGTPA | |
| | | ggttctacgctaagtctttgggttttagacaagatacctt | | WFVTLSLEGGAIND | |
| | | gattccttcggctggcatagacgagttcttcgaatatat | | VAEDATAYAHRDV | |
| | | cgctaaccataccgcaggtactcctgcctggtttgtga | | LFWVQLFMVNPVGP | |
| | | cccttagtttggaaggaggtgctattaacgacgtcgct | | ISDTTYEFTDGLYDV | |
| | | gaagatgctactgcttacgcacacagagatgttctattc | | LARAVPESVGHAYL | |
| | | tgggttcaattatttatggttaatccagtcggtccaatctc | | GCPDPRMEDAQQK | |
| | | tgacactacctatgaatttactgatggcttgtacgatgtg | | YWRTNLPRLQELKE | |
| | | ctagctagagctgttccagaatccgtcggtcatgcttac | | ELDPKNTFHHPQGV | |
| | | ttgggttgtccagatcccaggatggaagacgctcaac | | MPA | |
| | | aaaagtactggagaacaaatttaccaagattgcaaga | | | |
| | | attaaaagaagagcttgacccaaaaaacactttccatc | | | |
| | | accctcagggagttatgccagcc | | | |
| t808013 | Library | atgagatctcagttactacacggacttattggtctggttg | 69 | MRSQLLHGLIGLVA | 139 |
| | | ccttggtgtcaccttccttcgcagtccccacgaaacgt | | LVSPSFAVPTKREAV | |
| | | gaagctgtaacctcttgcttgacaaatgctaaggtccc | | TSCLTNAKVPIDAK | |
| | | aatagacgctaagggttcgcaaacttggacccaagat | | GSQTWTQDGTAYN | |
| | | ggtacagcctataacttgaggttacaatttgagccaatc | | LRLQFEPIAIAVPTTV | |
| | | gctattgccgttccaactactgttgctcaaatcagcgca | | AQISAAVACGSKHG | |
| | | gctgtcgcctgtggttctaagcatggcgtttccgtcagt | | VSVSGKSGGHSYTS | |
| | | ggtaaatctggtggtcactcctacacttctttgggtttgg | | LGLGGEDGHLVIEL | |
| | | gcggtgaagatggtcatcttgttattgaattggacagac | | DRLYSVKLAKDGTA | |
| | | tgtactcagtcaagttggctaaggatggaaccgctaag | | KIQPGARLGHVATE | |
| | | atccaaccaggtgctagattaggtcacgttgctactga | | LYNQGKRALSHGTC | |
| | | gttgtataaccagggtaaaagagcacttagtcatggta | | TGVGLGGHALHGG | |
| | | cctgtactggtgtaggtttgggtggtcacgctctacac | | YGMVSRKHGLTLDS | |
| | | ggcggatacggtatggtttccagaaagcatggtttaac | | IIGATVVLYDGKVV | |
| | | cttggactctataattggtgctactgtcgtcttgtacgac | | HCSKTERSDLFWAIR | |
| | | ggaaaagttgttcactgtagtaagacagaacgttccga | | GAGASFGIVAELEFN | |
| | | tttattctgggccattagaggtgcaggcgcttcttttggt | | TFPAPEQMTYFDIGL | |
| | | atcgtggctgaattagaatttaacaccttcccagcccct | | NWDQNTAAQGLWE | |
| | | gaacaaatgacctacttcgatattggtttgaattgggac | | FQEFGKTMPSEITMQ | |
| | | caaaacactgccgctcaaggtttgtgggaatttcaaga | | IAIRKDGYSIDGAYI | |
| | | atttggtaaaaccatgccttcagaaatcacgatgcaaat | | GDEAGLRKALQPLL | |
| | | tgctatacgtaaggatggatattctatcgatggtgcttac | | SKLNVQVSASTVSW | |
| | | atcggtgacgaagccggtttaagaaaggcacttcaac | | MGLVTHFAGTAEIN | |
| | | cattgttgagcaagttaaatgttcaagtctcggcttcga | | PTSASYDAHDTFYA | |
| | | ctgtgagctggatgggtctggttacacatttcgccggt | | TSLTTRELSLEQFKS | |
| | | actgctgagattaacccaacttctgcttcctatgatgca | | FVNSISTTGKSSSHS | |
| | | cacgacactttctacgctacttctttgacaaccagagaa | | WWVQMDIQGGKYS | |
| | | ttgtcattagaacaattcaagtcattcgtaaactccatca | | AVAKPKPTDMAYV | |
| | | gtaccaccggtaagtcaagttctcattcttggtgggtcc | | HRDALLLFQFYDSV | |
| | | agatggacattcagggtggcaaatactctgccgttgct | | PQGQTYPSDGFSLLT | |
| | | aagccaaaaccaacggatatggcttatgttcatagaga | | TLRQSISKSLRAGTW | |
| | | tgctttgcttttgtttcaattctacgattcagtgccccaag | | GMYANYPDSQLKA | |
| | | gtcaaacctacccatctgacggtttctccttactaactac | | DRAAEMYWGSNLP | |
| | | tctgagacaatccatttctaaatctcttagagccggcac | | RLQKIKAAYDPKNIF | |
| | | atggggtatgtatgcaaattacccagactcccaattga | | RNPQSVKPKA | |
| | | aggctgaccgtgctgctgaaatgtactggggtagcaa | | | |
| | | cctgcctagactacagaagattaaggctgcctatgatc | | | |
| | | ccaagaatatctttagaaatccacaaagtgttaagccta | | | |
| | | aggcc | | | |
| t808014 | Library | atgggaaacaccacatcaacttctgctggtcaatgtct | 70 | MGNTTSTSAGQCLL | 140 |
| | | attgtccgccgtgggtggcaatccagcattggtcgcttt | | SAVGGNPALVAFQN | |
| | | tcagaacgctcctttataccaagccgttgatgtaagac | | APLYQAVDVRPYNL | |
| | | cctataatctggacgttccagttactccagtcgctgttac | | DVPVTPVAVTTPET | |
| | | cacgccagaaactgtcgatcaagttgctagtatagtca | | VDQVASIVKCAADA | |
| | | aatgcgctgccgacgctggttacaaggttcaacctaa | | GYKVQPKSGGHSYG | |
| | | gtctggtggtcactcctacggtaactatggtttgggag | | NYGLGGVDGEVVV | |
| | | gtgtagacggtgaggttgtcgtcgatttaaaaaatttcc | | DLKNFQQFSMNNET | |
| | | aacaattctctatgaacaacgaaacctggagggctact | | WRATIGAGTLLGDV | |
| | | attggtgcaggtacattgcttggtgacgtgaccactcgt | | TTRLYNAGGRAMA | |
| | | ttgtacaacgccggtggcagagctatggcacatggta | | HGTCPQVGIGGHATI | |
| | | cctgtccacaagttggcatcggaggtcacgccactatt | | GGLGPTSRLWGAAL | |
| | | ggtggtttaggtccaacgtcgagattgtggggtgctgc | | DHIEEVQVVLANSSI | |
| | | cctagatcatatcgaagaagtgcaggttgttcttgctaa | | VRASQTENPDLLFA | |
| | | tagctctattgttagagcttcacaaactgagaaccctga | | LKGAGASFGIITEFT | |
| | | cttgttatttgctttgaagggtgctggtgcctccttcggt | | VRTEPAPGEAVQYS | |
| | | atcataacagaatttactgtccgtaccgaaccagctcc | | YTFNFGDNASKAKT | |
| | | aggcgaagcagttcaatattcatacaccttcaactttgg | | FKDWQAFVSTPNLN | |
| | | tgataatgcttccaaggctaagactttcaaagattggca | | RKFAATMTVLEDAI | |
| | | agccttcgtgtctacaccaaatttgaacagaaagttcg | | VASGTFFGTKEEFD | |
| | | ccgctaccatgactgtactggaagacgcaattgttgctt | | AFELESHFPENQGSN | |
| | | ctggtaccttctttggaactaaggaagaatttgatgcttt | | VTVVQDWLGLVAD | |
| | | cgaattggagtctcactttcctgaaaatcaaggttccaa | | WAEDAALEGGGGV | |
| | | cgtcacggtcgttcaggattggctgggtttagtcgctg | | PSAFYAKSLNFSPDT | |
| | | actgggcagaagatgcagctttggaaggaggtggtg | | LIPNDTIDDMFDYFS | |
| | | gtgtcccatccgctttctatgccaaaagtttgaatttcag | | TTEKDALLWFAIFDL | |
| | | tccagatactcttatccccaacgacacgattgatgacat | | SGGAVSDVPVHSTS | |
| | | gttcgactacttttctaccacagaaaaggatgctttgttg | | YTHRDTLFWLQSYA | |
| | | tggttcgccatttttgacctttcgggtggtgctgtgtctg | | ISVGPVSNTTIQFLD | |
| | | atgtccccgttcactcaacttcttacactcatagagatac | | GLSNLLTSSQPEVHF | |
| | | tctgttttggttacaatcgtacgcaatatctgttggccca | | GAYPGYVDPKLPDG | |
| | | gtaagcaacactactatccaattcttggacggtttgtcta | | QLAYWGSNLPKLEQ | |
| | | atttgctaacctcttcacaacccgaagttcactttggtgc | | IKAEVDPNDVFHNP | |
| | | ttatccaggttacgttgacccaaaattgccagacggac | | QSVKPAKQ | |
| | | aattagcttattggggttccaacttgccaaagctagagc | | | |
| | | aaatcaaggccgaagtagatcctaacgacgtgttccat | | | |
| | | aacccacaatccgttaaaccagctaagcaa | | | |
| t808021 | Library | atggctcagccaccttcctcagcattcgccacctgtcta | 71 | MAQPPSSAFATCLN | 141 |
| | | aatgatgtctgcggaggtcgtagtggctgtgtgggtta | | DVCGGRSGCVGYPS | |
| | | cccatcggacattttgtatcaaatcaactgggtagatag | | DILYQINWVDRYNL | |
| | | gtacaacttagacataaacttggagccagctgctgtta | | DINLEPAAVTKPEIT | |
| | | caaaaccagaaattacggaagatgtcgccgcttttatc | | EDVAAFIKCASENN | |
| | | aagtgtgctagcgaaaataacgtcaaggtacaagcca | | VKVQARSGGHSYA | |
| | | gatctggtggtcattcttacgctaatcacggtctgggtg | | NHGLGGEDGALVID | |
| | | gcgaagacggtgcattggttatcgatttagagaacttc | | LENFQHFSMNWDN | |
| | | caacacttttccatgaattgggacaactggcaagctac | | WQATIGAGHKLHD | |
| | | tattggagccggccataagcttcacgacgttactgaaa | | VTEKLHDNGGRAIS | |
| | | aactacatgataacggtggtagagctatctcacacggt | | HGTCPGVGLGGHAT | |
| | | acctgtcctggtgttggattgggtggtcatgctactattg | | IGGLGPSSRMWGSC | |
| | | gtggtttgggtccctcttctcgtatgtggggttcctgttta | | LDHVVEVEVVTADG | |
| | | gatcacgtcgttgaagtcgaagttgttactgctgacggt | | KIQRASEDENSDLFF | |
| | | aagattcaaagagcctctgaagatgaaaattcggactt | | ALKGAGASFGIITEF | |
| | | gttcttcgcactgaagggtgctggtgcttcatttggtata | | VMRTNEEPGDVVEY | |
| | | atcaccgaatttgtgatgagaacaaacgaagagccag | | TFSLTFSRHRDLSPV | |
| | | gcgatgttgtcgaatatacgttctctttgaccttctccag | | FEAWQNLISDPDLD | |
| | | acacagagacttgtccccagtttttgaagcttggcaaa | | RRFGSEFVMHELGAI | |
| | | acttgataagtgatccagatttagacagaagattcggtt | | ITGTFFGTEEEFEAT | |
| | | ccgagttcgttatgcatgaactaggtgctattatcactg | | GIPDRIPTGKKSIVV | |
| | | gtacctttttcggaactgaagaagaatttgaagcaactg | | NDWLGSVAQQAQD | |
| | | gtattcctgatcgtattccaaccggtaaaaagtctatcgt | | AALWLSDLSTAFTA | |
| | | tgtcaacgactggttgggttctgtcgctcaacaggccc | | KSLAFTKDQLLSSES | |
| | | aagatgccgctctttggctgagcgacttaagcaccgc | | IMDLMDYIDDANRG | |
| | | cttcactgctaaatctttggctttcaccaaggatcaattg | | TLIWFLIFDVTGGRI | |
| | | ttatcgtctgaaagtattatggaccttatggattacatcg | | NDVPMNATAYRHR | |
| | | atgacgctaacagaggtacattgatctggtttttgatctt | | DKVMFCQGYGIGIP | |
| | | cgatgtgactggaggtagaattaatgatgtacccatga | | TLNGRTREFIEGINSL | |
| | | acgccaccgcctataggcacagagacaaggttatgtt | | IRSSVPTNLSTYAGY | |
| | | ctgccaaggttacggcataggtatcccaactttgaacg | | VDASLESPQDSYWG | |
| | | gtaggacaagagagtttattgagggtataaattccttga | | PNLDALGQVKEDW | |
| | | tcagaagttctgtgcctaccaatttgtccacttacgctg | | DPSDLFSNPQSVRPG | |
| | | gttacgtcgatgcatctttagaatctccacaggactcct | | QKSVVDYFDNRASS | |
| | | attggggtccaaacctagacgctttgggacaagttaaa | | NGSEDSSGGSNGGT | |
| | | gaagactgggacccatccgatctgttttcaaatccaca | | RDEQGGCWSWRRS | |
| | | atctgttagacccggtcaaaagtccgtagttgattatttc | | GPAFAVFVALFVGF | |
| | | gataacagagcttcgtctaatggttcagaagacagctc | | PTPQTSWVQKQNLR | |
| | | tggtggcagtaatggaggtacccgtgatgaacaaggt | | DPALDLTDAESPSRT | |
| | | ggttgttggtcttggagaagatccggtccagcatttgct | | PVVNPNTLTTDTMA | |
| | | gtctttgttgctttattcgtaggtttccctactccacaaact | | KLSRGAPGGKLKMT | |
| | | tcttgggtccaaaagcagaacttgcgtgacccagcttt | | LGLPVGAVMNCAD | |
| | | agatctgacagacgccgaatcaccttccagaacacct | | NSGARNLYIISVKGI | |
| | | gttgttaacccaaacacgttaacaactgacaccatggc | | GARLNRLPAGGVGD | |
| | | caagttgtctcgtggcgctccaggtggtaaattaaaga | | MVMATVKKGKPEL | |
| | | tgactttgggtttgcccgtcggtgccgttatgaactgcg | | RKKVHPAVIVRQSK | |
| | | ctgacaattcgggtgcaagaaacctttacattatttcgg | | PWKRFDGVFLYFED | |
| | | tcaaaggaatcggtgctagattgaacagactaccagc | | NAGVIVNPKGEMKG | |
| | | tggtggtgttggtgatatggttatggctactgttaagaa | | SAITGPVGKEAAEL | |
| | | gggtaaaccagagttgagaaagaaggttcatccagc | | WPRIASNSGVVM | |
| | | cgtcatagtcagacaaagtaagccatggaaacgttttg | | | |
| | | atggtgttttcttgtacttcgaagacaatgccggtgttatt | | | |
| | | gtgaacccaaaaggagaaatgaagggaagcgctatc | | | |
| | | actggtcctgttggtaaggaagctgccgaattgtggcc | | | |
| | | aagaattgcttctaattcaggtgtcgtcatg | | | |
| t808022 | Library | atgggaaattcggccagcgtggcaggtagagcttgttt | 72 | MGNSASVAGRACFV | 142 |
| | | tgtcgctgctgtaggtcatgatcccaacttggttacattc | | AAVGHDPNLVTFRG | |
| | | aggggtgacttactatatgagttccgtattcagccatca | | DLLYEFRIQPSYNLA | |
| | | tacaaccttgccataccagttcaccctacggtcgtcac | | IPVHPTVVTYPKTTA | |
| | | ctacccaaaaactaccgctcaagttgctgaaatcgtttc | | QVAEIVSCAAAQNY | |
| | | ttgcgccgctgcacaaaattataagatgcaagcctaca | | KMQAYSGGHSYGN | |
| | | gtggcggtcactcttacggtaactacggtttgggtgga | | YGLGGEDGHVVVD | |
| | | gaagatggtcatgttgttgtcgacttgaagaacttccaa | | LKNFQDFTMDPDTH | |
| | | gactttactatggatccagatactcacgttgctaccattg | | VATIGAGTSLGDLQ | |
| | | gcgctggtacttccttaggtgatctgcaagacagattgt | | DRLWHAGGRAMAH | |
| | | ggcacgctggtggtagagcaatggcccatggtagttg | | GSCPQVGVGGHFTI | |
| | | tcctcaagtgggtgtcggtggtcacttcaccatcggtg | | GGLGMMSRQWGMS | |
| | | gcttgggcatgatgtccagacagtggggtatgtctctg | | LDHVVEAQVVLANS | |
| | | gaccatgtcgttgaagctcaagtagtcttggccaattct | | SVVTASDTQNQDIF | |
| | | tctgtggttacggcttccgatactcaaaaccaagatattt | | WAIKGAAASFGIVT | |
| | | tttgggccatcaagggtgctgctgcttcgtttggtattgt | | KFKVRTHGVPKAAI | |
| | | tacaaaattcaaggtaagaacacacggtgttccaaag | | QYQYTFSQGDVLDK | |
| | | gccgctatccaatatcagtacaccttctctcaaggtgac | | VKLFMAWQNIVAKP | |
| | | gtattagacaaagttaagttgtttatggcttggcaaaac | | NLTRNFSTELTIFQD | |
| | | attgtcgctaagccaaatttgactcgtaacttcagtactg | | GIMIMGSFFGTRDEF | |
| | | aattgaccatattccaagatggaatcatgattatgggta | | HKFELENDLPLQGL | |
| | | gctttttcggtactagagatgaatttcataagttcgagtt | | GNVAYITNWLSLVA | |
| | | agaaaatgatttaccccttcaaggccttggtaatgttgc | | HTAEDYLLRLTGNV | |
| | | atatatcaccaactggctatccttggttgctcataccgct | | LTSFYAKSLSFTADE | |
| | | gaagactacttgttgagactgacaggtaacgtcttgac | | LFNEQGLVTLFTYL | |
| | | ttctttttacgccaaatctctatcattcacggctgacgaat | | DAAPKGTPTWWVIF | |
| | | tgttcaacgagcaaggtcttgttactttgttcacttattta | | DLEGGATNDVPVNA | |
| | | gacgcagctccaaaaggcacacctacctggtgggtta | | TSYAHRDAIMWMQ | |
| | | tcttcgatttggaaggaggtgccactaacgatgtccca | | SYAVAGFEPPGFIIK | |
| | | gttaacgctacttcttacgcccacagagatgctataatg | | RFLNRLHGVVIGNR | |
| | | tggatgcaaagttacgccgtcgctggttttgaaccacc | | APGAVRSYPGYVDP | |
| | | aggttttattattaagagattcctaaacagattgcatggt | | YLRNAQETYWGPNL | |
| | | gttgtaatcggtaatcgtgcacctggtgctgtccgttcc | | ARLQDIKTAVDPDD | |
| | | tatcctggttatgtcgacccatacttaagaaatgcccag | | VFHNPQSVKVNSLS | |
| | | gaaacctactggggtccaaacttggctagattacaag | | PPDPGSHDV | |
| | | atattaagacagctgttgatccagatgacgtttttcacaa | | | |
| | | tccacaatccgttaaggtgaatagtctttcgccaccag | | | |
| | | accctggaagccatgatgtc | | | |
| t808024 | Library | atgggtcaaacgccaagctctcctctagccgactgttt | 73 | MGQTPSSPLADCLN | 143 |
| | | aaatgcagtttgcaacggaagagataactgtgtggctt | | AVCNGRDNCVAFPS | |
| | | ttccatccgctccactgtatcagatctcttgggtcgaca | | APLYQISWVDRYNL | |
| | | ggtacaatttggatatagaagtagagcccattgctgtta | | DIEVEPIAVTRPETA | |
| | | ccagaccagaaactgccgaagacgtttcaggtttcgt | | EDVSGFVKCAAAHN | |
| | | caaatgtgctgccgctcacaacattaaggttcaagcaa | | IKVQAKSGGHSYAN | |
| | | agtccggcggtcattcttacgctaactatggtcttggtg | | YGLGGEDGELVVDL | |
| | | gtgaagatggtgaattggtcgttgatttgagaaatttcc | | RNFQDFSIDTNTWQ | |
| | | aagattttagtatcgatacaaacacttggcaagccacct | | ATFGAGHKLDDVTE | |
| | | tcggcgctggtcacaagttagacgacgtcactgaaaa | | KLHKNGKRAISHGT | |
| | | attgcataagaacggtaagcgtgctatttcacacggta | | CPGVGIGGHATIGGL | |
| | | cttgccctggtgtcggtatcggtggtcacgctaccattg | | GPESRMWGSCLDHV | |
| | | gcggattaggtcctgagtctcgtatgtggggttcgtgtt | | IGVEVVTADGSIVHA | |
| | | tggatcatgtgatcggtgtagaagtcgttactgctgac | | SDTENSDLFFALKG | |
| | | ggaagcatagttcatgcctcggacaccgaaaattccg | | AGASFGIVTSFVVKT | |
| | | atttgttctttgctcttaaaggcgcaggagcttctttcggt | | RPEPGSVVQYSYSV | |
| | | attgtaacatcttttgttgttaagactagaccagaaccag | | TFAKHADLSPVFRQ | |
| | | gttccgttgtccaatacagctactctgtcacgttcgcaa | | WQELVMDPGLDRR | |
| | | aacacgctgacctatccccagttttcagacaatggcag | | FGTEFTMHELGVIIS | |
| | | gaattggtaatggatccaggtttggacagaagatttgg | | GTFYGTDEEFQATGI | |
| | | taccgaatttaccatgcacgagctgggtgtcattatctc | | PDRIPKGKISVVFDD | |
| | | tggtactttctatggtactgacgaagagttccaagccac | | WMAVIAKHAEEAA | |
| | | aggtattcctgatagaatcccaaagggtaagatttctgt | | LSLSSISSAFTARSLA | |
| | | tgttttcgatgattggatggctgttatagcaaaacacgc | | FRREDKISPETITNL | |
| | | cgaagaagctgctttgtcgttaagtagtatctcctctgct | | MNYIDSADRGTLVW | |
| | | tttaccgcccgttccttggctttcagaagagaagacaa | | FLIFDATGGAISDVP | |
| | | gatctcaccagaaactatcaccaacctgatgaactaca | | TNATAYSHRDKVM | |
| | | ttgattctgctgatagaggtactttggtctggttcctaatc | | YCQGYGVGIPTLNQ | |
| | | tttgatgctaccggtggtgccatttccgatgtcccaaca | | QTKDFLSGIINTIQSG | |
| | | aacgccacagcttactcacatagagacaaggttatgta | | AGNTLTTYPGYVDP | |
| | | ctgtcaaggctacggcgtaggtatacccactttaaatc | | ALTNPQESYWGPNI | |
| | | aacagaccaaggacttcttgtcgggtattattaacacta | | DTLRAIKSQWDPNDI | |
| | | tacaatctggtgccggtaatactttgactacttatcctgg | | FHNPQSVRPAAVAA | |
| | | ttatgtcgatccagctttgaccaacccacaagaatccta | | | |
| | | ctggggaccaaacatcgacactttaagagctatcaag | | | |
| | | agtcagtgggatccaaacgatatctttcataatccacaa | | | |
| | | tctgttaggccagctgccgtggctgcc | | | |
| t808026 | Library | atgcttaaaaccatcgctgccgttgtattcatttgctcgc | 74 | MLKTIAAVVFICSQA | 144 |
| | | aggcttttttggtccgtgcagacctaaagtccgagctg | | FLVRADLKSELTAL | |
| | | actgctttgggcgtgggtgccgtcttccctggagattc | | GVGAVFPGDSVYTS | |
| | | agtttacacgagcgatgctaagccatataacttgagatt | | DAKPYNLRFDFKPA | |
| | | tgacttcaaaccagctgctataacttttcccaatacccc | | AITFPNTPADVSQIV | |
| | | agccgatgtctctcaaattgttcaaatcgccggtaagta | | QIAGKYAHKVAPRG | |
| | | cgcacacaaggttgcaccaagaggtggtggtcattcc | | GGHSYISNGVGGMD | |
| | | tacatttctaacggtgttggtggaatggacaatagtatc | | NSIIADMSHFKSIVV | |
| | | attgctgatatgtctcacttcaagtctattgtagtccatac | | HTNNDTATIETGNR | |
| | | aaacaatgacactgctaccatcgaaactggtaacaga | | LGDIALALFQYGRG | |
| | | ttaggcgatatagctttagctttgttccaatatggtaggg | | MPHGACPYVGIGGH | |
| | | gtatgcctcacggtgcttgtccatacgtaggtattggtg | | ANFGGFGFISRSWGL | |
| | | gccacgccaactttggtggtttcggtttcatctcaagat | | TLDVVEAIDLVLAN | |
| | | cctggggtttgaccctagatgttgtcgaagctattgacc | | GTITTVSATQNPDLY | |
| | | tggttttagcaaacggcactatcacgacagtctctgcta | | WAMRGSGSSFGITT | |
| | | ctcaaaacccagacttgtattgggccatgagaggtag | | AIHVRTFSAPASGIIA | |
| | | cggtagttcttttggaatcaccaccgctatccatgttag | | LDTWYLNLEQAVR | |
| | | aaccttctccgcaccagcttctggtattatcgctttggac | | ALSSFQDFAHNTVT | |
| | | acttggtacttgaatcttgaacaagctgttagagccttg | | LPSYFGGEFVVNAG | |
| | | agttcctttcaagatttcgctcacaatactgtgactttacc | | PSPGLLSITFFSGFW | |
| | | atcttattttggtggtgaatttgtcgttaacgccggtcctt | | GPPNQYNSTLAPWK | |
| | | ccccaggtttgttgtctattacattcttctcgggattttgg | | NSMPFPPNTTSYSQG | |
| | | ggtcctccaaatcagtacaactctacgctagcaccatg | | NYIESLSARFGGAPL | |
| | | gaaaaattccatgccattccccccaaacacaacttcat | | DTSLGPDNTDTFYV | |
| | | actcgcaaggtaactacatagaaagcttgtccgcccgt | | KSLIVPQVTISDEGA | |
| | | ttcggaggtgctcccttggatacctctctaggtccagat | | QVGISDKAWRALFQ | |
| | | aatactgacactttttacgtcaagtcattaatagtcccac | | YLINEQPNLPVDWFI | |
| | | aagttaccatttctgatgaaggtgctcaagtaggtatta | | EVELWGGQNSAINA | |
| | | gcgataaagcttggagagctctgttccaatatttgataa | | VPQASTAFAYRDLL | |
| | | acgagcagcctaacctgcctgttgattggttcatcgaa | | WTLQMYSYTPNHQP | |
| | | gttgaattatggggtggtcaaaatagtgccattaacgc | | PYPDAGFAFNDGMA | |
| | | cgtcccacaagcttctacagcttttgcttatagagacttg | | NSIIHNMPNGWNYG | |
| | | ttgtggactttgcaaatgtactcttacaccccaaaccat | | AYTNYVDNRLDDW | |
| | | caaccaccttacccagacgccggttttgcattcaatga | | QRLYYANHYPALQA | |
| | | cggcatggctaatagtatcattcataacatgccaaacg | | LKSRYDPSDTFSFPT | |
| | | gttggaattatggtgcttacactaattacgttgataaccg | | SIELL | |
| | | tttagacgattggcagagattgtactatgctaaccacta | | | |
| | | ccccgctttgcaagccttgaagtctaggtatgacccta | | | |
| | | gtgatacattttcgttcccaacttccattgaactttta | | | |
| t808029 | Library | atgactaccaacggtatacaacccggccatgtcggta | 75 | MTTNGIQPGHVGNL | 145 |
| | | atttaacacaggaccaagaggctaaacttcaacaattg | | TQDQEAKLQQLWSI | |
| | | tggtcgattgtactaacgttgttagatgttaagtccttgc | | VLTLLDVKSLQGGD | |
| | | aaggtggagatacttctgcccagacccaaccagacc | | TSAQTQPDQRPSTSL | |
| | | aacgtccaagtactagcttgtctagggctgacaccgtt | | SRADTVVSAHGQTA | |
| | | gtgtcagcacacggtcaaactgcttttaccgaagatct | | FTEDLSQVLRENGM | |
| | | atcccaagttttgagagaaaacggtatgtctaatccag | | SNPDIKSVRESLSNT | |
| | | atatcaagtccgtcagagaatctctgtccaacacttcta | | SIDELRSGLLYTAKH | |
| | | tcgacgaattgagatccggtttattgtacacagccaaa | | DSPDVLLLRFLRAR | |
| | | cacgattcacctgatgtcttgcttctaagattcttaagag | | KWDVGKAFGMMLR | |
| | | ctcgtaagtgggacgttggtaaggctttcggtatgatgt | | ALVWRKDQHVDDK | |
| | | tgagagcattggtatggagaaaagatcaacatgttgac | | VIANPELAALVTSQN | |
| | | gacaaggttattgctaatccagagctggccgctttggt | | TVDTHAAKECKDFL | |
| | | cacttctcagaacaccgtcgatacacacgccgctaag | | DQMRMGKCYMHGT | |
| | | gaatgtaaggattttctggaccaaatgagaatgggtaa | | DRDGRPVLVVRVRF | |
| | | atgctatatgcatggtaccgatagggacggaagacct | | HQPSKQSEAVINRFI | |
| | | gttttagttgttagagtcagattccaccaaccatctaagc | | LHTIETARLLLAPPQ | |
| | | aaagtgaagccgtgattaaccgttttatcttgcacacga | | ETVTIIFDMTGFGLS | |
| | | tcgaaacagctagattgctattggctccaccacaagaa | | NMEYAPVKFIIECFQ | |
| | | actgtcactattattttcgacatatggaccggtttcggttt | | ENYPESLGYMLIHN | |
| | | gtctaatatggaatacgcccctgttaaatttattatagaa | | APWVFSGIWKIIKG | |
| | | tgtttccaagaaaactatccagaatcgttaggctacatg | | WMDPVIVSKVNFTN | |
| | | cttattcataatgctccctgggttttttccggtatctggaa | | KVSDLEKFIAPEQIV | |
| | | gatcatcaagggttggatggatccagtcatagtgtcta | | KELKGKEDWTYEY | |
| | | aagtgaacttcactaacaaggtttcggatttagaaaaat | | VEPVAGENELMADT | |
| | | tcatcgctccagagcaaattgtaaaggaactaaaggg | | ETRDRIYAERLKIGE | |
| | | taaggaggactggacctacgaatatgtcgaacccgta | | ELLLRTSEWVSTSQR | |
| | | gcaggcgagaacgaattgatggctgacactgaaacc | | KDAAATTTAREQRS | |
| | | agagataggatttacgcagaaagattgaagatcggtg | | ETIESLRQNYWQLD | |
| | | aagagttgttgttgagaaccagcgaatgggtttccactt | | PYVRGRTFLDRTGV | |
| | | cacagcgtaaggacgctgctgccacgactacagcta | | VKPGGKIDFYPSPDL | |
| | | gagaacagcgttctgaaaccatagaaagtttgagaca | | EPSTAKMLEVEHFE | |
| | | aaattattggcaactagacccttacgttagaggtagaa | | RTQFDPYLFLLPHGA | |
| | | cttttttggatagaactggtgttgtgaagcctggaggta | | RIAVRHCSVTALPTY | |
| | | agattgacttctacccatctccagatttggagccaagta | | LKAHPRGMLSTMAF | |
| | | ctgccaaaatgttagaagtcgaacactttgaaagaacc | | SFLRVLSSLLLVLQL | |
| | | caatttgatccataccttttcttattgccacacggtgcta | | STAASTSTLRQCLLT | |
| | | gaattgctgttaggcattgtagcgtcaccgctttaccaa | | AVQNDPTLVAVDG | |
| | | cctatcttaaggctcacccacgtggtatgctatctacaa | | DLLFQTLAVQVYNL | |
| | | tggccttcagtttcctacgtgtattgtcttccctattgctg | | NWPVTPAAVAFPKS | |
| | | gtcttgcaattatcaaccgctgctagtacttcgacgttg | | AQQVSSIVNCAASL | |
| | | agacaatgtcttttgactgctgttcaaaacgacccaacc | | GYKVQAKSGGHSY | |
| | | ctggttgccgttgatggagatttgcttttccaaaccttgg | | GNYGLGGTNGAISIN | |
| | | ctgttcaagtctacaacttgaactggccagtcactcctg | | LKNMKSFSMNYTN | |
| | | ctgctgtagcctttcccaaatccgcccagcaagtttctt | | YQATVGAGMLNGE | |
| | | ctatcgttaattgcgcagcatcccttggttataaagttca | | LDDYLHNAGGRAIA | |
| | | agctaagtcgggtggtcattcttacggtaactatggctt | | HGTSPQIGVGGHATI | |
| | | aggtggtacaaacggcgcaatctctataaaccttaaaa | | GGLGPAARQYGME | |
| | | atatgaagtcattctcaatgaattacactaactaccaag | | LDHVLEAEVVLANG | |
| | | ctacggttggtgctggtatgttgaatggagagttagac | | TVVRASSTQNSDLLF | |
| | | gattatctgcacaatgccggtggtagagcaattgctca | | AIKGAGASFGVVTE | |
| | | tggcacaagcccacaaattggtgtcggtggtcacgca | | FVFRTEPEPGSAVQY | |
| | | actatcggtggtttgggtcctgctgccagacagtacgg | | SFTFGLGSTSSRADL | |
| | | tatggaattagatcacgtcttggaagctgaagttgtgtt | | FKKWQSFISQPDLTR | |
| | | agcaaatggtacagtcgtcagagcttcctctacccaaa | | KFASICTILDHVLVIS | |
| | | actcggacttgttgtttgccatcaagggagctggtgctt | | GTFFGTKAEYDALG | |
| | | ctttcggtgtggtgactgaatttgtttttagaacagagcc | | LEDQFPGHTNSTVIV | |
| | | agaacctggatctgctgttcagtactccttcacttttggtt | | FTDWLGLVAQWAE | |
| | | taggctccacctcttcacgtgccgacctattcaagaag | | QSILDLTGGIPADFY | |
| | | tggcaatcattcatttctcaaccagacttgactagaaaa | | SRCLSFTEKTPIPSTG | |
| | | ttcgccagcatctgtaccatcttggaccatgttttggtca | | VDQLFEYLDSADTG | |
| | | tttccggtactttctttggtactaaagctgaatacgacgc | | ALLWFVIFDLEGGAI | |
| | | tttaggtttagaagatcaatttccaggtcacaccaattct | | NDVPMDATGYAHR | |
| | | actgtgatcgtatttaccgattggttgggactggttgctc | | DTLFWLQSYAITLGS | |
| | | aatgggctgaacaatctattttggatttgaccggtggta | | VSQTTYDFLDRVNEI | |
| | | ttccagccgatttctactccagatgtttatcttttactgaa | | IRNNTPGLGNGVYP | |
| | | aagactccaattccatcgactggtgtcgatcaattgttc | | GYVDPRLQNAREAY | |
| | | gagtatctggacagtgcagatacgggagctctattgtg | | WGSNLPRLMQIKSL | |
| | | gtttgttattttcgatttggagggtggtgccattaacgat | | YDPSDLFHNPQGVL | |
| | | gtcccaatggatgctacaggttacgctcatagagaca | | PA | |
| | | ccttgttttggttacagtcttatgccataactttaggttctg | | | |
| | | tttcccaaactacctacgacttcctggatcgtgttaacg | | | |
| | | aaataattagaaataacacaccaggtttgggaaacggt | | | |
| | | gtttacccaggttacgtcgaccctagacttcagaatgc | | | |
| | | aagagaagcttattggggttccaatttgccaagacttat | | | |
| | | gcaaattaaaagcctttatgacccatcggacctgttcca | | | |
| | | caacccccaaggtgttttgcctgct | | | |
| t808039 | Library | atgggccagggtcaatcctctgccggtggtttgcaag | 76 | MGQGQSSAGGLQD | 146 |
| | | actgcttaacgtcagcagtgggtagcggaaatctagct | | CLTSAVGSGNLAVP | |
| | | gtaccttctaaacccttctaccaacaaactgatgtcaag | | SKPFYQQTDVKPYN | |
| | | ccatataacttggatatccacgtccatccagttgctgtta | | LDIHVHPVAVTYPQ | |
| | | catacccacaaactaacgaggacgttgctgctattgtc | | TNEDVAAIVRCAKE | |
| | | agatgtgctaaggaacacgaagccaaagtccagcca | | HEAKVQPRSGGHSY | |
| | | cgttccggtggtcattcgtacggtaattttgccaccggt | | GNFATGNGNDNMIV | |
| | | aacggaaacgataacatgatagttgttgacttgaagca | | VDLKHFKQFSMDDN | |
| | | cttcaagcaattctctatggatgacaatacctggatcgc | | TWIATLGSGHLLGD | |
| | | aactttaggttccggccaccttctgggtgatgtcacaaa | | VTKKLLANGGRAM | |
| | | gaaattgttagctaacggtggtagggctatggctcatg | | AHGTCPQVGIGGHA | |
| | | gtacttgtcctcaagttggtattggcggtcacgctacca | | TIGGLGPMSRMWGS | |
| | | ttggtggtctaggtccaatgtctaggatgtggggcagtt | | SLDHVQEITVVLANS | |
| | | ccttggaccacgttcaagaaatcactgtggtcttggcc | | SIITASPTQNKDVFW | |
| | | aattctagcattatcacggcctctccaacccaaaataa | | AMKGAGASFGIITEF | |
| | | ggatgttttttgggctatgaagggtgcaggagcctcatt | | KVITHPAPGEAVKY | |
| | | cggtataattactgaatttaaagttattacccatccagct | | SFGFSGGSHRDQAK | |
| | | ccaggtgaggctgttaagtatagtttcggtttttcggga | | RFKKWQSMIADPGL | |
| | | ggttcacacagagatcaagctaagagattcaaaaagt | | SRKLASQVVLSEIG | |
| | | ggcaatctatgatcgctgaccctggattgagtagaaaa | | MIISGTFFGTQAEYN | |
| | | cttgcttctcaagtagttctgagtgaaatcggtatgatta | | QLNLTSVFPEMSSH | |
| | | tatcaggtacctttttcggtacccaggctgaatacaacc | | KIIVFNDWAGLVGH | |
| | | aattgaacttaacttctgtcttccctgaaatgtcctcccat | | WAEDVGLQLGGGIS | |
| | | aagattatcgtatttaacgattgggctggtctagtgggt | | SPFYSKSLAFTPNDLI | |
| | | cactgggccgaagacgtgggtttacaattgggtggtg | | PAEGIDRFFEYLDEV | |
| | | gaatctcttctccattctactccaagagcttggctttcac | | DKGTLIWFGIFDLEG | |
| | | cccaaacgacttgattcctgctgaaggtattgacagatt | | GATNDIPADATAYG | |
| | | tttcgaatatttggatgaagttgataagggtactttgatct | | HRDALFYFQSYGVN | |
| | | ggtttggtatattcgatttggaaggtggcgccactaac | | LGLKVKDETRDFIN | |
| | | gatattccagcagacgcaactgcatacggtcatagag | | GMNSVLEGSLSNHK | |
| | | atgcattgttttatttccagtcatatggtgtcaatctagga | | LGAYAGYVDPALSL | |
| | | ttaaaggttaaggatgagacaagagactttatcaatgg | | EAAQVGYWGDNLP | |
| | | tatgaatagcgtccttgaaggttctttgagcaaccacaa | | RLRQIKRAVDPDDV | |
| | | actgggtgcttacgctggttacgttgatcccgctctttct | | FHNLQSVRPAAS | |
| | | ttggaagccgcccaggttggttactggggtgacaactt | | | |
| | | accacgtctgagacaaattaagagagctgtagatcca | | | |
| | | gacgacgttttccataatttgcaatccgtcagaccagct | | | |
| | | gcttcc | | | |
| t808040 | Library | atgggtaataagccatccactcctttagcccattgcttg | 77 | MGNKPSTPLAHCLR | 147 |
| | | agagatgtttgtgcaggaaggggtaactgtgtcgcttt | | DVCAGRGNCVAFPN | |
| | | cccaaacgagtatctttaccaggctaactgggtaaaac | | EYLYQANWVKPYN | |
| | | cctacaatttggacgtgccagttaagccaattgctgtct | | LDVPVKPIAVFRPDN | |
| | | ttagacctgataatgccgctgacgtcgctgctgctgtta | | AADVAAAVKCAGQ | |
| | | agtgtgccggtcaatcatcggttcacgttcaagcaaaa | | SSVHVQAKSGGHSY | |
| | | tctggtggccactcttatgcaaacttcggtctaggtggt | | ANFGLGGGDGGLMI | |
| | | ggtgatggtggtttgatgatcgacctgcaacatttgaac | | DLQHLNKFSMNNET | |
| | | aagtttagcatgaacaacgaaacctggcaagctacatt | | WQATFGSGFLLGDL | |
| | | cggatccggtttcctattgggcgatttagacaagcaac | | DKQLHANGNRAMA | |
| | | tgcacgctaatggtaatcgtgccatggctcatggtactt | | HGTCPGVGIGGHATI | |
| | | gcccaggtgttggcataggtggtcacgccaccatcgg | | GGIGPSSRMWGTAL | |
| | | aggtattggtccatcttccagaatgtggggtacggcttt | | DHVLEVEVVTADGK | |
| | | agatcacgtattggaagtcgaagttgtgactgctgatg | | IQRASKTQNSDLFW | |
| | | gtaaaattcaaagagccagtaagacccagaactctga | | GLQGAGASFGIITEF | |
| | | cttgttttggggtttgcaaggtgctggtgcttcattcggc | | VVRTEPEPGSVVEY | |
| | | atcataactgaatttgttgtccgtaccgaacctgaacca | | AYSLNFGKQADMAP | |
| | | ggttctgtcgttgagtacgcctactctctaaatttcggca | | VYKKWQDLVGDPN | |
| | | aacaagcagatatggctccagtgtataagaagtggca | | LDRRFTSLFIAEPLG | |
| | | agaccttgtgggtgaccctaacttagatagaagattca | | VLITGTFYGTLDEYK | |
| | | ccagtttgtttattgccgaaccattgggtgttttgatcact | | ASGIPDKLPASGASI | |
| | | ggtacattctacggtaccctagacgaatacaaggcttc | | TVMDWLGSLAHIAE | |
| | | cggaatcccagacaagttgcccgcttcgggtgcctcc | | KTGLYLSNVSTKFV | |
| | | attacagtcatggattggttgggtagcttagctcacatc | | SRSLALREEDLLSEQ | |
| | | gctgaaaaaactggtttatatttgtctaacgtatctacta | | SIDDLFKYMGSADA | |
| | | aatttgtttccagatcattagcattaagggaagaggacc | | DTPLWFVIFDNEGG | |
| | | ttttgagcgaacagtccattgatgatttgtttaagtacat | | AIADVPDNSTAYPH | |
| | | gggctctgctgacgctgacacaccattgtggttcgttat | | RDKIILYQSYSVGLL | |
| | | tttcgataacgaaggtggtgccatcgctgatgtccctg | | GVSDKMINFVDGIQ | |
| | | ataattctactgcttatccacatagagacaagattatact | | DLVQKGAPNAHTTY | |
| | | gtaccaaagttactccgttggtttgttgggagtttctgac | | AGYINANLDRNAAQ | |
| | | aagatgataaatttcgtcgatggtattcaagatcttgtac | | KFYWGDKLPQLQQL | |
| | | aaaagggcgctcctaacgcccacacgacttacgctg | | KKKFDPTSLFSNPQS | |
| | | gttatatcaacgctaacttagacagaaatgctgcccaa | | IDPAD | |
| | | aaattttattggggtgacaagttgccacagctgcaaca | | | |
| | | actaaagaagaagttcgacccaacatcgttattcagca | | | |
| | | atccacaatctattgatccagccgat | | | |
| t808041 | Library | atgggtaacaccacttccatcgcagccggcagagatt | 78 | MGNTTSIAAGRDCL | 148 |
| | | gtttggtttcagctgtcggtccagctcatgtgacatttca | | VSAVGPAHVTFQDA | |
| | | agacgctctgctttatcagacgaccgccgttgatcctta | | LLYQTTAVDPYNLN | |
| | | caatttgaacattcccgtaactccagctgctgtcacata | | IPVTPAAVTYPQSAE | |
| | | cccacaatcggccgaagagatagctgctgttgtcaaa | | EIAAVVKCASDYDY | |
| | | tgcgcttccgactatgattacaaggttcaagcacgtagt | | KVQARSGGHSFGNY | |
| | | ggaggtcacagcttcggtaattacggtctaggtggtca | | GLGGQNGAIVVDM | |
| | | aaacggtgccatcgtcgttgacatgaagcacttctctc | | KHFSQFSMDESTFV | |
| | | aattttctatggatgaatctactttcgttgctaccattggt | | ATIGPGTTLGDLDTE | |
| | | ccaggtactacgttaggcgacttggataccgaactata | | LYNAGGRAMAHGIC | |
| | | taatgctggtggtagggccatggcccatggtatctgtc | | PTIRTGGHLTVGGLG | |
| | | ctactattagaactggcggtcacttaaccgtcggtgga | | PTARQWGLALDHIE | |
| | | ttgggtccaacagccagacagtggggtctggctttgg | | EVEVVLANSSIVRAS | |
| | | atcatattgaagaggtagaagttgttttggctaactcttc | | NTQNQDILFAVKGA | |
| | | catcgtgagagcatcgaacactcaaaatcaagacattt | | AASFGIVTEFKVRTQ | |
| | | tattcgctgtaaagggtgcagctgcttcttttggtatagt | | EAPGLAVQFSFTFNL | |
| | | caccgaatttaaagttagaactcaagaagctccaggtt | | GSPAQKAKLVKDW | |
| | | tggctgttcaattctccttcaccttcaacttgggttctcct | | QAFIAQENLSWKFY | |
| | | gcacaaaaggctaagctagtcaaagattggcaagcat | | SNLVIFDGQIILEGIF | |
| | | ttattgctcaggaaaacttgagctggaagttctactcaa | | FGSKEEYDELDLEK | |
| | | acttggtcatcttcgacggtcaaataatcttagaaggta | | RFPTSEPGTVLVLTD | |
| | | ttttctttggatcgaaagaggaatacgacgaactagatt | | WLGMIGHALEDTIL | |
| | | tggaaaagagatttccaacgtcagagcccggcactgt | | KLVGDTPTWFYAKS | |
| | | tttggttttaacagattggctgggcatgatcggacacgc | | LGFTPDTLIPDSAIDD | |
| | | tttggaagatactattttgaagttggtgggtgacacccc | | FFDYIHKTNAGTLA | |
| | | aacgtggttttatgctaagtccctgggtttcactccaga | | WFVTLSLEGGAINS | |
| | | cactcttatcccagattctgccattgatgacttcttcgact | | VSEDATAYGHRDVL | |
| | | acatccacaagactaacgctggtaccttagcttggttc | | FWFQVFVVNPLGPIS | |
| | | gtaaccttgtcattggaaggtggtgcaattaattctgttt | | QTTYDFTNGLYDVL | |
| | | cggaagatgctacagcttatggtcatagagatgtcttgt | | AQAVPESAGHAYLG | |
| | | tttggtttcaagttttcgttgttaatcctttaggtcctattag | | CPDPKMPDAQRAY | |
| | | tcaaaccacgtacgatttcactaacggcctgtatgacg | | WRSNLPRLEDLKGD | |
| | | tccttgcccaagccgtaccagaatccgccggtcacgc | | LDPKDTFHNPQGVQ | |
| | | ttacctaggttgtccagaccctaaaatgccagacgctc | | VGP | |
| | | aacgtgcctactggcgtagtaacttgccaagacttgaa | | | |
| | | gatttgaagggtgacttggacccaaaggatactttcca | | | |
| | | taatccacagggtgttcaagttggtcca | | | |
| t808045 | Library | atgctgtcaaccatggcattcagctttgtccttagaatttt | 79 | MLSTMAFSFVLRILS | 149 |
| | | atctccattgttcttgatcctacaattatctactgccgcta | | PLFLILQLSTAASTST | |
| | | gtacatccactttgaggcagtgtttgttaaccgctgttca | | LRQCLLTAVQNDPT | |
| | | aaatgaccctacgttggtagctgttgatggtgatttgct | | LVAVDGDLLYQTLA | |
| | | gtaccaaactcttgccgtgcaagtctataacttgaactg | | VQVYNLNWPVTPA | |
| | | gccagttacccccgctgctgtcgcctttccaaagtcga | | AVAFPKSTQQVASIV | |
| | | ctcaacaagttgcttctatagttaactgcgctgcatcctt | | NCAASLGYKVQAKS | |
| | | gggatacaaagtgcaagctaagtctggcggtcattcct | | GGHSYGNYGLGGT | |
| | | acggtaattatggtttgggtggtaccaatggtgccattt | | NGAISINLKNMKSFS | |
| | | caatcaacttaaagaacatgaaatcgttctctatgaact | | MNYTNYQATVGAG | |
| | | acacgaattaccaagccacagttggtgctggtatgctt | | MLNGELDEYLHNA | |
| | | aacggcgagttagacgaatatttgcacaacgctggtg | | GGRAVAHGTSPQIG | |
| | | gtcgtgctgtcgcacacggaacttcccctcagattggt | | VGGHATIGGLGPSA | |
| | | gtaggtggtcatgctactattggaggactaggtccatc | | RQYGMELDHVLEAE | |
| | | ggctagacaatacggtatggaattggatcacgtcttag | | VVLANGTVVRASST | |
| | | aagccgaagttgttttggcaaacggtaccgtagtccgt | | QNSDLLFAIKGAGA | |
| | | gcttcttctactcagaatagcgacttgctgttcgccatca | | SFGVVTEFVFRTEPE | |
| | | agggtgctggtgctagttttggtgtcgttacagagtttgt | | PGSAVQYTFTFGLGS | |
| | | gttcagaacagaaccagaaccaggttctgctgttcaat | | TSARADLFKKWQSF | |
| | | ataccttcactttcggcttgggttccacctctgccagag | | ISQPDLTRKFASICTL | |
| | | ccgatctatttaagaaatggcaatccttcatatcccaac | | LDHVLVISGTFFGTK | |
| | | cagacctgactagaaagtttgcaagtatctgtaccttgt | | EEYDALGLEDQFPG | |
| | | tagatcatgttttggtcatttctggtactttctttggtacaa | | HTNSTVIVFTDWLG | |
| | | aagaagaatacgacgctttgggcttggaagatcaattt | | LVAQWAEQSILDLT | |
| | | cccggacacactaactctactgttatcgttttcaccgatt | | GGIPADFYARCLSFT | |
| | | ggttgggtttggtggctcaatgggctgaacaatcaattt | | EKTLIPSNGVDQLFE | |
| | | tagacctgactggtggtatcccagctgatttctacgcaa | | YLDSADTGALLWFV | |
| | | gatgtttgagctttactgaaaagaccctaattccttccaa | | IFDLEGGAINDVPMD | |
| | | tggtgtcgaccaattattcgagtacctagactcagcag | | ATGYAHRDTLFWLQ | |
| | | atactggtgctttgttatggttcgtcatctttgatcttgaa | | SYAITLGSVSETTYD | |
| | | ggtggtgccattaacgacgtcccaatggacgctaccg | | FLDNVNEIIRNNTPG | |
| | | gctatgctcacagagataccttgttttggctacagtctta | | LGNGVYPGYVDPRL | |
| | | cgctattacgcttggttctgttagtgagactacctacgat | | QNAREAYWGSNLPR | |
| | | ttcttggacaatgtaaacgaaatcataagaaacaatac | | LMQIKSLYDPTDLFH | |
| | | accaggacttggtaacggtgtttaccctggttatgttga | | NPQGVLPA | |
| | | tccaaggttgcaaaatgcaagagaagcctattggggtt | | | |
| | | caaatcttccacgtttgatgcaaattaagtctctatatga | | | |
| | | cccaaccgacttgtttcataacccacaaggtgttttgcc | | | |
| | | tgcc | | | |
| t808046 | Library | atggctccatccatttcattttctttgctacaaatctcgctt | 80 | MAPSISFSLLQISLLA | 150 |
| | | ttggcctattctggtctggtgagtggagatttctctttaa | | YSGLVSGDFSLRQC | |
| | | gacagtgcttggaatccgctgttagcagggtagcattc | | LESAVSRVAFEGDPF | |
| | | gagggcgaccctttttaccaattattgtcagtcagacca | | YQLLSVRPYNLDISI | |
| | | tacaacttagatatatccattgttccagctgccgtcgctt | | VPAAVAFPADTNEV | |
| | | tccccgctgacactaatgaagttgcagctgtcgtaaga | | AAVVRCAAQNGYQ | |
| | | tgtgctgcccaaaacggttatcaagttcaagcaaaaag | | VQAKSGGHSYANH | |
| | | tggtggtcactcatacgctaatcatggtttgggtggtac | | GLGGTNGAVVVNLE | |
| | | caacggagctgttgtggttaatctggaaaacttgcaac | | NLQHFSMNTTTWEA | |
| | | acttctccatgaacacgactacctgggaagccacaat | | TIGAGTLLGDVTKR | |
| | | cggtgctggtacattattgggtgatgtcaccaagcgttt | | LSDAGGRAMAHGT | |
| | | gtctgacgctggcggtagagcaatggcccatggtact | | CPQVGSGGHFTIGGL | |
| | | tgtcctcaggttggttctggaggtcactttactattggtg | | GPSSRQFGAALDHII | |
| | | gcctaggtccatctagtagacaatttggcgccgctttg | | EAEVVLANSSIIRAS | |
| | | gatcatatcatagaagctgaagtcgttctagctaactctt | | ETENPDVFFAVRGA | |
| | | ctattatcagagcatctgagactgaaaacccagatgtg | | ASGFGIVTEFKVRTE | |
| | | ttcttcgctgtaagaggagctgcttccggttttggtattg | | PEPGQAVRYSYSFSF | |
| | | ttaccgaatttaaggttcgtaccgaaccagaaccaggt | | SDTATRADLFKKWQ | |
| | | caagccgtcagatacagttattctttctcgttcagcgac | | AYVTQPDLPRELAS | |
| | | accgctacgcgtgcagacttgttcaagaaatggcaag | | TLTILEHGMFITGTFF | |
| | | cctacgtcactcaaccagatttgcctagagaacttgctt | | GSKEEYNALKIETEF | |
| | | ctactctgacaattttggaacacggtatgttcatcactg | | PGFAKGGTLVLDDW | |
| | | gtacgtttttcggttcaaaggaggagtacaatgctctaa | | LGLVSNWAEDLLLS | |
| | | agattgaaaccgaatttcccggtttcgccaagggtgga | | EEEIEQMFEYIDNVD | |
| | | accttagtcttggatgactggttgggtttagttagtaatt | | KGTLLWFAIFDLQG | |
| | | gggctgaagacttgcttttgtcggaagaagaaatcga | | GAVGDVPVDATAY | |
| | | gcaaatgttcgaatatattgataacgttgacaaaggtac | | AHRDTLIWLQSYAI | |
| | | actactgtggtttgccattttcgacctacaaggtggtgct | | NLFGRISETTVEFLE | |
| | | gtcggtgatgtaccagtcgatgccactgcttacgctca | | RLNELTLTSTAKTVP | |
| | | cagagataccttgatatggctacaatcctacgcaatca | | YAAYPGYVDPRLTD | |
| | | atctgtttggtagaataagcgaaactactgttgagttttt | | AQAAYWGSNLARL | |
| | | agaacgtttgaacgaattgactttgacatctacagctaa | | NRIKAEIDPNNVFHN | |
| | | gacggttccatatgcagcctaccctggttatgttgaccc | | PQSVRPASG | |
| | | aagattgactgatgctcaagctgcctactggggatcga | | | |
| | | acttagctagattgaacagaatcaaagctgaaatcgac | | | |
| | | ccaaacaatgtattccacaatccccaatccgttcgtcca | | | |
| | | gcttctggt | | | |
| t808051 | Library | atgggtaatactacctcgatagccgctggcagagattg | 81 | MGNTTSIAAGRDCL | 151 |
| | | cctggttaacgctgtcggtggtaaccaggcattagtag | | VNAVGGNQALVAF | |
| | | cttttcaagaccaattgctatatcaatccacggccgtcg | | QDQLLYQSTAVEAY | |
| | | aagcttacaacttgaatattcctgttacaccagctgctgt | | NLNIPVTPAAVTFPE | |
| | | cactttcccagagtcttcagaacaaatcgcagccgtgg | | SSEQIAAVVKCASEH | |
| | | ttaaatgtgcttctgaacacgactacaaggttcaagctc | | DYKVQARSGGHSFG | |
| | | gtagcggtggacatagtttcggtaattatggtttgggtg | | NYGLGGTNGAIVVD | |
| | | gtaccaacggcgccatcgtggttgatatgaagaaattt | | MKKFDQFSMDESSY | |
| | | gatcaattctccatggacgaatcgtcttacattgctacta | | IATIGPGTTLGDVDT | |
| | | ttggtcccggtaccactttaggtgatgtcgacacagaa | | ELYNAGGRAMAHGI | |
| | | ttgtacaacgctggaggtagagccatggctcacggta | | CPTIRTGGHLTMGG | |
| | | tttgtccaaccatcagaactggcggtcatcttacgatgg | | LGPTARQWGLALDH | |
| | | gtggtttgggtccaactgccaggcagtggggcttggc | | IEEVEVVLANSSIVR | |
| | | tctggaccacatagaagaggttgaagtcgtattagcta | | ASHTQNQDILFAVK | |
| | | attcttccatcgttagagcatctcatacccaaaaccaag | | GASASFGIVTEFKVR | |
| | | atattttgtttgccgttaagggtgcttccgcatcattcggt | | TEPAPGLAVQYSYT | |
| | | attgtcactgaatttaaggttagaactgaacctgcacca | | FNLGSAASKAKLVK | |
| | | ggtttggctgtccaatactcttataccttcaatttgggta | | DWQEFIAQDNLTWK | |
| | | gtgcagcctccaaggctaaattagttaaggattggcaa | | FYSNMVIIDGDIILEG | |
| | | gagttcatcgctcaggacaacttgacatggaaattctat | | IFFGSKEEFDALELE | |
| | | agcaatatggtcattatcgacggagatataattctggaa | | NRFPPKNPGNILVLT | |
| | | ggtatctttttcggttctaaggaagaatttgatgctttaga | | DWLGMISHSLEDIIL | |
| | | actagaaaacaggttcccacccaagaacccaggtaa | | RVAGGVPTYFYAKS | |
| | | catacttgtgttgactgattggttgggaatgatttctcact | | LGFTPQALIPSSAIDD | |
| | | ccttggaagacatcattttaagagttgctggtggtgtac | | LFDYIEKTNPGTLA | |
| | | caacctacttttacgctaagtccttaggtttcacacctca | | WFITLSLEGGAINNV | |
| | | agctttgatcccatctagcgctattgatgacctgttcgat | | PADATAYGHRDVLH | |
| | | tatatagaaaagactaatccaggtactctagcctggttt | | WVQIFAANPLGPISE | |
| | | atcaccttgtccttggagggcggagctattaacaacgt | | TTYDFTDGLYNILA | |
| | | tccagctgacgcaacagcctacggtcacagagatgtg | | KAVPESAEHAYLGC | |
| | | cttcattgggtccaaatctttgccgctaatcctttgggtc | | PDPRMKDAQKAYW | |
| | | caatttctgaaaccacttacgacttcactgacggtttata | | RDNLPRLEELKAEL | |
| | | caacatccttgctaaagccgttcctgagtctgctgaac | | DPKDTFHNPQGVAV | |
| | | atgcttatttaggttgtcctgatccacgtatgaaagacg | | A | |
| | | ctcaaaaggcttactggagagataacctgccacgtttg | | | |
| | | gaagaattaaaggctgaattggatcccaaagatactttt | | | |
| | | cacaatccacaaggtgtagccgtcgct | | | |
| t808061 | Library | atgttattgaaactatttttcttggccgtagcagcttcagt | 82 | MLLKLFFLAVAASV | 152 |
| | | tgctctggctgcttccagtgaggccttgaagcagtgctt | | ALAASSEALKQCLE | |
| | | ggaaaacgtcttcactgaccgtgcaggctttgctttcg | | NVFTDRAGFAFAGD | |
| | | ccggtgatttattctatgacagaatagttaatagatacaa | | LFYDRIVNRYNLNIP | |
| | | cttgaatatcccagtcaccccttcggctttggcttttcca | | VTPSALAFPTSSQQV | |
| | | acgagctctcaacaagttgccgatattgtgaagtgtgc | | ADIVKCAADNGYPV | |
| | | agctgataacggttaccccgttcaagctaggtccgga | | QARSGGHSYGNYGL | |
| | | ggtcattcttatggtaactacggtcttggtggtgctgac | | GGADGAVAIDLKHL | |
| | | ggcgccgtcgctatcgatttaaaacacctacaacaatt | | QQFSMDKTTWQATI | |
| | | ctctatggacaagacaacttggcaggctaccattggtg | | GAGSLLSDVTQRLS | |
| | | ccggatctttgctatccgatgttacccaaagattgagcc | | HAGGRAMSHGICPQ | |
| | | acgctggtggcagagccatgtctcatggtatttgtcca | | VGSGGHFTIGGLGPT | |
| | | caagtcggttcgggtggtcacttcacaatcggtggttt | | SRQFGAALDHVLEV | |
| | | gggaccaacttcaagacaatttggtgctgccttagacc | | EVVLANSSIVRASDT | |
| | | atgttcttgaagtcgaagtcgttttggctaattccagtatt | | ENKDLFWAIKGAAS | |
| | | gtccgtgcttctgatactgaaaacaaggatttgttttgg | | GYGIVTEFKVRTEPE | |
| | | gctattaagggtgctgcatctggatacggtatcgttacc | | PGTAVQYAYSMEFG | |
| | | gaatttaaagtgagaactgaacctgaaccaggtaccg | | NPTKQATLFKSWQA | |
| | | ctgttcaatatgcatacagcatggagttcggtaatccaa | | FVSDPKLTRKMAST | |
| | | ctaagcaagcaacccttttcaagtcctggcaggcttttg | | LTMLENSMAISGTFF | |
| | | tgtctgacccaaaattgactagaaagatggcctctaca | | GTKEEYDKLNLTNK | |
| | | ttaacgatgctggaaaacagtatggctatatccggtact | | FPGANGDALVFEDW | |
| | | ttcttcggtactaaggaagaatacgacaagttgaatttg | | LGLVAHWAEDLILG | |
| | | accaacaagtttcctggtgctaatggtgacgctttagttt | | LAAGIPTNFYAKSTS | |
| | | tcgaagattggctgggcctagtggctcactgggctga | | WTPQTLITPETVDK | |
| | | ggatttgatattgggtttagctgccggtattccaactaac | | MFDYIATVNKGTLG | |
| | | ttctatgccaaatcaacgtcttggactccccaaacatta | | WFLLFDLQGGYTND | |
| | | atcacccccgaaaccgtagataaaatgtttgactacat | | IPTNATSYAHRDVLI | |
| | | cgccaccgttaacaaaggtactcttggctggttcttatt | | WLQSYTVNFLGPISQ | |
| | | gtttgacttgcaaggtggttatacgaacgatattccaac | | AQIDFLDGLNKIVTN | |
| | | caacgccacatcatacgctcacagagatgtcttgattt | | NKLPYTAYPGYVDP | |
| | | ggctacaatcttatacagttaactttttgggtcctatctcc | | LMPNAPEAYWGTN | |
| | | caggctcaaattgacttcctagatggtttgaataagatt | | LPRLQQIKELVDPND | |
| | | gtcaccaacaataagttgccatacactgcttacccagg | | VFRNPQSPSPANKEP | |
| | | ttacgttgatccattgatgccaaatgctccagaagcata | | L | |
| | | ctggggaactaacttgccaagattacaacaaatcaag | | | |
| | | gaattagtcgaccctaatgatgtttttcgtaacccacaat | | | |
| | | ctccatccccagctaacaaagagccactg | | | |
| t808069 | Library | atgggtaacggaaatagcacaccttttcgtgactgttta | 83 | MGNGNSTPFRDCLD | 153 |
| | | gattctatatgcgcaaacagatccacctgtgtgacgtat | | SICANRSTCVTYPGD | |
| | | ccaggtgacccactgttctcgtgttggagtaggccctt | | PLFSCWSRPFNLEFP | |
| | | caatttggagtttcctgtagtcccagccgctatcattag | | VVPAAIIRPETTTEV | |
| | | accagaaactaccactgaagttgctgaaactgttaaat | | AETVKCAKKYGYK | |
| | | gtgctaagaagtacggttacaaggttcaggctaaatca | | VQAKSGGHSYGNH | |
| | | ggtggccactcctacggtaaccatggtttgggtggtgt | | GLGGVGGAVSIDMV | |
| | | cggaggtgccgtcagtattgatatggtcaacctaaga | | NLRDFSMNNKTWY | |
| | | gatttctctatgaacaataagacctggtatgcttctttcg | | ASFGSGMNLGELDE | |
| | | gttctggtatgaaccttggtgaattggacgagcacttac | | HLHANGRRAIAHGT | |
| | | atgccaacggcagaagagcaatcgctcacggtacat | | CPGVGTGGHLTVGG | |
| | | gcccaggtgttggtactggtggtcatttgaccgttggtg | | LGPISRQWGSALDH | |
| | | gtttgggtccaatttccagacaatggggctctgctctgg | | LLEIEVITADGTVQR | |
| | | accacttgctagaaatcgaagtcatcactgctgatggt | | ASYTKNSGLFWALR | |
| | | acggtgcaaagagcctcatatactaaaaattctggatt | | GAGASFGIVTKFMV | |
| | | attttgggctttgcgtggtgctggcgcctctttcggtatt | | KTHPEPGRVVQYSY | |
| | | gttacaaagtttatggttaagactcacccagaacctggt | | NIALASHAETAELYR | |
| | | agagtagtgcaatactcatacaatatagctttggcctcc | | EWQALVGDPNMDR | |
| | | catgctgaaactgctgaactatatagggaatggcaag | | RFSSLFVVQPLGALI | |
| | | ccttggttggagatccaaacatggaccgtagattctctt | | TGTFFGTKSQYQAT | |
| | | ccttattcgtcgtccaaccattgggtgctttgattaccgg | | GIPDRLPGADKGAV | |
| | | taccttctttggtaccaagtcccaataccaggcaactg | | WLTDWAGLLLHEA | |
| | | gtattcctgacagactaccaggtgctgataaaggtgct | | EAAGCALGSIPTAFY | |
| | | gtctggcttacagattgggcaggcttgttattgcacgaa | | GKSLSLSEQDLLSDS | |
| | | gctgaggccgctggttgtgccttaggtagcatcccaa | | AITDLFKYLEDNRSG | |
| | | ccgctttctacggcaagtcgttgtctttgagtgaacaag | | LAPVTILFNTEGGA | |
| | | accttttatcagattctgctattaccgacttgtttaagtatt | | MMDTPANATAYPH | |
| | | tagaggataacagatccggtttagcccccgttactatct | | RNSIIMYQSYGIGVG | |
| | | tgtttaataccgaaggtggtgctatgatggatacgcctg | | KVSAATRKLLDGVH | |
| | | ccaacgccactgcttacccccacagaaactccattatc | | ERIQRSAPGALSTYA | |
| | | atgtaccaatcttatggtataggagttggtaaggttagt | | GYIDAWADRKAAQ | |
| | | gctgcaacacgtaaactgttggacggtgttcatgaaag | | KLYWADNLPRLREL | |
| | | aatccaaagaagcgcaccaggcgctctgtctacttac | | KKVWDPADVFSNPQ | |
| | | gctggttatattgacgcctgggctgaccgtaaggctgc | | SVEPAD | |
| | | ccaaaagctatactgggctgataatttgccaagattaa | | | |
| | | gagaattaaaaaaggtctgggatccagcagatgttttc | | | |
| | | tcaaacccacagtctgttgagccagcagac | | | |
| t808076 | Library | atggattctaacacttgggaggccacgttcggctcag | 84 | MDSNTWEATFGSGF | 154 |
| | | gatttttacttggtgaactagacaaacatttgcacgctaa | | LLGELDKHLHANGN | |
| | | tggtaacagggctatggcacacggtacctgtccaggt | | RAMAHGTCPGVGM | |
| | | gttggtatgggtggtcatgccactatcggaggtattgg | | GGHATIGGIGPSSRL | |
| | | ccctagctccagactgtggggtacaaccttagaccac | | WGTTLDHVLQVEV | |
| | | gtattgcaggtcgaagtggttactgctgatggtaagat | | VTADGKIQRASKTQ | |
| | | acaacgtgcttctaagactcaaaacccagatttgttctg | | NPDLFWALQGAGAS | |
| | | ggctctacaaggtgctggtgcctcgtttggcattatcac | | FGIITEFVVRTEPEPG | |
| | | cgaatttgtcgttagaaccgaacccgaaccaggtagt | | SVVEYTYSVSLGKQ | |
| | | gttgtcgaatacacctattccgtatctttgggaaagcaa | | SDMAPLYKQWQAL | |
| | | tctgacatggctccattgtacaaacaatggcaagctttg | | VGDPSLDRRFTSLFI | |
| | | gttggtgatccttccctggacagaagattcacaagttta | | AEPLGVLITGTFYGT | |
| | | ttcattgccgagccattgggtgttttaatcactggtacat | | MYEWHASGIPDKLP | |
| | | tttatggtactatgtacgaatggcacgcatcaggtatcc | | RGPISVTVMDSLGSL | |
| | | ctgataagttgccaagaggtccaatttcggtcaccgtta | | AHIAEKTGLYLTNV | |
| | | tggactctttgggatctttagctcatattgccgaaaaaa | | PTSFASRSLALRQQD | |
| | | ctggcctgtacttgaccaatgtcccaacgtccttcgcta | | LLSEQSIDDLFEYMG | |
| | | gcagatctcttgccttgagacagcaagatttgttgtccg | | SANADTPLWFVIFD | |
| | | agcaatctatcgatgacttattcgaatatatgggttcgg | | NEGGAIADVPDNST | |
| | | ctaacgcagacactccactttggttcgtgatctttgaca | | AYPHRDKVIVYQSY | |
| | | acgaaggtggtgctattgctgatgtgcctgataatagc | | SVGLLGVTDKMIKF | |
| | | accgcctacccacatagagataaggttattgtttaccaa | | LDGVQDIVQRGAPN | |
| | | agctactccgtcggtttactaggtgtcactgataaaatg | | AHTTYAGYINPQLD | |
| | | ataaagttcttggacggtgttcaagatattgtccagagg | | RKAAQQFYWGDKL | |
| | | ggagctcccaacgcccacacgacctatgcaggttac | | PRLQQIKKQYDPNN | |
| | | atcaatccacaattggaccgtaaggctgctcaacaatt | | VFCNPQSIYPAEDMS | |
| | | ctattggggtgacaagctaccaagattgcaacagatta | | DG | |
| | | agaagcaatatgatcctaacaacgtgttttgcaatccac | | | |
| | | aatctatctacccagctgaagacatgtctgacggt | | | |
| t808093 | Library | atgggtaacacaacttccatcgcaggcagagattgctt | 85 | MGNTTSIAGRDCLV | 155 |
| | | agtctcagcccttggaggtaattctgctttggctgctttc | | SALGGNSALAAFPN | |
| | | ccaaaccaattgctgtggaccgccgacgttcacgagt | | QLLWTADVHEYNL | |
| | | ataatttgaacctacctgtaacgccagctgccataacct | | NLPVTPAAITYPETA | |
| | | accccgaaactgctgaacagattgctggtatcgttaag | | EQIAGIVKCASDYD | |
| | | tgtgctagtgattacgactataaagtgcaagctaggtct | | YKVQARSGGHSFGN | |
| | | ggtggtcattcctttggtaattacggtttgggaggtact | | YGLGGTDGAVVVD | |
| | | gatggtgccgttgtcgtcgacatgaagcacttcaacca | | MKHFNQFSMNDQT | |
| | | attctcgatgaacgatcaaacctacgaagcagttattg | | YEAVIGPGTTLNDV | |
| | | gtccaggtactaccttaaacgacgttgacattgaattgt | | DIELYNNGKRAMAH | |
| | | acaacaatggcaagagagctatggctcatggtgtttgt | | GVCPTIKTGGHFTIG | |
| | | ccaactatcaaaacaggtggtcactttacaattggcgg | | GLGPTARQWGLALD | |
| | | tctgggtcctactgccagacaatggggtttggctttaga | | HVEEVEVVLANSSIV | |
| | | tcacgtcgaagaagtggaagtagtcttggccaactctt | | RASNTQNQDVFFAV | |
| | | ctatcgttcgtgctagcaatacccaaaaccaggatgtc | | KGAAADFGIVTEFK | |
| | | ttctttgctgtcaagggcgcagctgccgacttcggtatc | | VRTEPAPGLAVQYS | |
| | | gttacggagttcaaggttagaactgagccagcacctg | | YTFNLGSTAEKAQF | |
| | | gtttagctgttcaatattcgtatacctttaatcttggtagta | | VKDWQSFISAKNLT | |
| | | ctgctgaaaaagcccaatttgtcaaggattggcaaag | | RQFYNNMVIFDGDII | |
| | | cttcatttccgctaaaaacttgactcgtcaattctacaac | | LEGLFFGSKEQYDA | |
| | | aatatggttatatttgacggtgacattattttagaaggttt | | LGLEDHFAPKNPGNI | |
| | | gtttttcggatcaaaggaacaatacgatgccttgggttt | | LVLTDWLGMVGHA | |
| | | ggaagatcattttgctccaaagaatccaggtaacatcc | | LEDTILKLVGNTPTW | |
| | | tagtgctgacggactggttgggaatggtaggtcatgct | | FYAKSLGFRQDTLIP | |
| | | ttggaagacaccattttgaagctagttggaaacacacc | | SAGIDEFFEYIANHT | |
| | | cacttggttctacgctaaatctttgggtttcagacaagat | | AGTPAWFVTLSLEG | |
| | | accctaatcccatctgctggtattgacgaatttttcgaat | | GAINDVAEDATAYA | |
| | | atatagcaaaccacaccgctggtactccagcttggttc | | HRDVLFWVQLFMV | |
| | | gttaccttatctctggaaggcggcgctataaacgatgt | | NPLGPISETTYEFTD | |
| | | ggctgaagatgccacagcatacgcacacagagatgt | | GLYDVLARAVPESV | |
| | | cctattttgggttcagttgttcatggtcaatccactaggt | | GHAYLGCPDPRMEN | |
| | | ccaatctcagaaactacctacgagttcactgacggttta | | APQKYWRTNLPRLQ | |
| | | tatgacgtcttagcaagagctgtccctgaatctgttggt | | ELKEELDPKNTFHHP | |
| | | catgcctatttgggttgtccagacccaagaatggaaaa | | QGVIPA | |
| | | cgctccacaaaagtactggcgtactaatttgcctagatt | | | |
| | | acaagaattgaaagaggaattggatccaaagaacac | | | |
| | | cttccaccatccacaaggtgtgattccagct | | | |
| t808094 | Library | atgggtaacactacgtcgattgccgcaggcagagatt | 86 | MGNTTSIAAGRDCL | 156 |
| | | gccttgtcagtgctgttggtggtgtggctgctcatgttg | | VSAVGGVAAHVAF | |
| | | cttttcaggactctttgttataccaagccacagccgtag | | QDSLLYQATAVELY | |
| | | agctgtataatctaaacatacctgtcacccccgctgctg | | NLNIPVTPAAVTYPQ | |
| | | ttacttacccacaaagcaccgatgaaatcgccgctgtc | | STDEIAAVVKCASD | |
| | | gttaaatgtgcttcagactatgactacaaggttcaagct | | YDYKVQARSGGHSF | |
| | | cgttccggtggtcactccttcggaaactacggtttgggt | | GNYGLGGQNGAIVI | |
| | | ggccaaaatggtgcaattgtaatcgatatgaagcactt | | DMKHFSQFSLDKST | |
| | | ctctcaattttctttagataagtctactttcattgccacctt | | FIATFGPGTTLGNLD | |
| | | cggtccaggtactacattgggaaacttggacaccgaa | | TELYHAGNRAMAH | |
| | | ctatatcatgctggtaacagagcaatggctcacggtat | | GICPTIRTGGHLTMG | |
| | | ctgtccaactattagaaccggaggtcatttgacaatgg | | GLGPAARQWGLAL | |
| | | gcggtttgggtccagctgccaggcagtggggtttggc | | DHVEEVEVVLANSS | |
| | | attagatcacgttgaagaagtcgaagttgtccttgctaa | | VVRASDTQNQDVFF | |
| | | ttccagcgtggtaagagcctctgacactcaaaatcaag | | AVKGAAASFGIVTE | |
| | | acgttttctttgctgttaaaggtgctgctgcttcttttggta | | FKVRTEEAPGLAVQ | |
| | | tcgtcactgagttcaaggttcgtactgaagaagcccct | | YSFPFNLGTPAEKAK | |
| | | ggtttggctgttcaatacagctttccattcaacttgggta | | LVKDWQAFIAQENL | |
| | | ccccagctgaaaaagctaagttagttaaggattggca | | SWKFYSNMVIFDGQ | |
| | | agcatttatagctcaagaaaatttatcgtggaagttctac | | IILEGIFFGSKKEYDE | |
| | | tcaaacatggtcatctttgatggtcaaattattctggagg | | LDLENKFPTSEPGTV | |
| | | gcattttcttcggctccaaaaaggaatatgacgaattgg | | LVLTDWLGMIGHGL | |
| | | acctggaaaacaagttccccacctcggaaccaggtac | | EDTILRLVGNSPTWF | |
| | | agtcttggtcttgaccgattggcttggtatgatcggtca | | YAKSLGFTPSTLISD | |
| | | cggtttggaagacactattttaagattggtgggtaactc | | SAIDGLFDYIHKTNP | |
| | | cccaacatggttctacgccaagtctcttggctttactcct | | GTLAWFVTLSLEGG | |
| | | tctactttaattagtgatagtgctatcgatggtttgttcgat | | AINTVSEDATAYGH | |
| | | tatatccacaaaaccaacccaggtacattggcctggttt | | RDVLFWVQIFVANP | |
| | | gttacgctatctttggagggtggagctataaatactgtc | | LGPISQTTYDFADGL | |
| | | tccgaagatgccactgcttacggacatagagatgtttt | | YNVLAQAVPDSAGH | |
| | | gttctgggttcaaatctttgttgctaaccctttgggtcca | | AYLGCPDPKLPDAQ | |
| | | atttcacagactacctacgacttcgctgacggattatac | | RAYWRSNLPRLEEL | |
| | | aacgttctggctcaagctgtgccagattctgccggtca | | KRDLDPKDIFYNPQ | |
| | | tgcttacctaggttgtccagaccctaaattgccagatgc | | GVQIVS | |
| | | tcagagagcatactggaggtctaatctaccaagactg | | | |
| | | gaggaacttaagagagacttggatccaaaagacatct | | | |
| | | tctataatccacaaggtgtccaaattgtttcc | | | |
| t808103 | Library | atgggtaacaccacatcgatcgctgccggacgtgact | 87 | MGNTTSIAAGRDCL | 157 |
| | | gcttactttccgcagtcggtggcaatcatgctcacgtg | | LSAVGGNHAHVAFQ | |
| | | gctttccaagatcagctgctataccaagccactgctgtt | | DQLLYQATAVEPYN | |
| | | gaaccttataacttgaatataccagtaacgcccgctgc | | LNIPVTPAAVTYPQS | |
| | | cgttacttacccacaatcagctgacgaggttgctgccg | | ADEVAAVVKCAAD | |
| | | tcgttaaatgtgcagctgattacggttataaggtccaag | | YGYKVQARSGGHSF | |
| | | ctagaagtggtggtcactcttttggtaactacggtttgg | | GNYGLGGEDGAIVV | |
| | | gtggcgaagatggtgctattgttgtggacatgaagcat | | DMKHFDQFSMDEST | |
| | | ttcgatcaattttctatggacgaatctacctatacagcca | | YTATIGPGITLGDLD | |
| | | ctattggtccaggtatcactttgggcgatttggacaccg | | TALYNAGHRAMAH | |
| | | ctttatacaatgcaggtcacagagccatggctcacggt | | GICPTIRTGGHLTIGG | |
| | | atttgtccaaccatcaggacgggtggtcacttgactata | | LGPTARQWGLALDH | |
| | | ggaggtttaggtcctactgctagacagtggggacttgc | | VEEVEVVLANSSIVR | |
| | | cttggatcatgtagaagaggttgaagtcgttctggctaa | | ASDTQNQEILFAVK | |
| | | cagctccattgtcagagcctctgacacacaaaaccaa | | GAAASFGIVTEFKVR | |
| | | gaaatcttgttcgccgttaagggtgctgctgcttccttc | | TEEAPGLAVQYSFTF | |
| | | ggaatcgtcaccgaatttaaagttcgtactgaagaagc | | NLGTAAEKAKLVKD | |
| | | tccaggtttggctgtccaatactccttcacctttaatttgg | | WQAFIAQEDLTWKF | |
| | | gtactgctgccgagaaggcaaagttagttaaagattg | | YSNMNIIDGQIILEGI | |
| | | gcaagccttcattgctcaagaagatcttacttggaagtt | | YFGSKAEYDALGLE | |
| | | ctattctaacatgaacataattgacggtcaaatcattctg | | EKFPTSEPGTVLVLT | |
| | | gaaggtatctatttcggttcaaaagccgaatacgacgc | | DWLGMVGHGLEDV | |
| | | tttaggtttggaagaaaagtttccaacttctgagccagg | | ILRLVGNAPTWFYA | |
| | | caccgtgttggtactaactgactggttgggtatggttgg | | KSLGFAPRALIPDSAI | |
| | | tcatggtttggaagatgtaattttaagattggtcggtaat | | DDFFEYIHKNNPGT | |
| | | gctcccacctggttctacgctaagtcgctaggttttgca | | VSWFVTLSLEGGAI | |
| | | ccaagagctctaattcctgattccgcaatagacgattttt | | NKVPEDATAYGHRD | |
| | | tcgaatacattcacaaaaacaatccaggtaccgtttcat | | VLFWVQIFMINPLGP | |
| | | ggtttgttaccttgtctttggaaggcggtgccatcaaca | | VSQTIYDFADGLYD | |
| | | aggtccccgaagatgctactgcttatggccatagagat | | VLAKAVPESAGHAY | |
| | | gttctattctgggtccagattttcatgatcaacccattgg | | LGCPDPRMPNAQQA | |
| | | gtccagtttctcaaacaatttacgatttcgctgacggtct | | YWRNNLPRLEELKG | |
| | | gtatgacgtcttagctaaggcagtgccagaaagcgcc | | DLDPKDIFHNPQGV | |
| | | ggtcacgcatacttgggttgtccagatcctcgtatgcct | | MVVS | |
| | | aacgctcaacaagcctactggagaaacaacttgccaa | | | |
| | | gactggaagagttaaagggtgatcttgacccaaaaga | | | |
| | | cattttccataatccacaaggtgtcatggttgtctcc | | | |
| t808125 | Library | atgggtaacggacagtccacccccttgcaacaatgttt | 88 | MGNGQSTPLQQCLN | 158 |
| | | aaatactgtttgcaacggtagactaggttgtgtagctttc | | TVCNGRLGCVAFPS | |
| | | ccaagtgatgcattgtatcaagccgcttgggtcaagcc | | DALYQAAWVKPYN | |
| | | ttacaacctggacgtgccagttacgcctatcgctgttttt | | LDVPVTPIAVFKPSS | |
| | | aaaccaagctctacagaggatgtcgccggtgctataa | | TEDVAGAIKCAVAS | |
| | | agtgtgctgttgcctcgaatgtgcacgttcaagcaaag | | NVHVQAKSGGHSY | |
| | | tccggtggccattcttacgctaacttcggtttgggtggt | | ANFGLGGQDGELMI | |
| | | caagacggagaattaatgattgacttggctaaccttca | | DLANLQDFHMDKTS | |
| | | ggattttcacatggacaaaacttcttggcaagctactttc | | WQATFGAGYRLGD | |
| | | ggtgctggttataggttaggcgatttggataagaagttg | | LDKKLQANGNRAIA | |
| | | caagccaatggtaatagagccattgctcatggtacctg | | HGTCPGVGIGGHATI | |
| | | tccaggagtcggtatcggtggtcacgccactattggtg | | GGLGPMSRMWGSA | |
| | | gtctaggtccaatgtcacgtatgtggggcagtgctttg | | LDHVLSVQVVTADG | |
| | | gaccatgtcttatctgttcaagtagtgaccgctgatggtt | | SIKNASESENSDLFW | |
| | | ccatcaaaaacgcatccgaatctgaaaactcagatctg | | ALRGAGASFGVITKF | |
| | | ttttgggctttgagaggagctggtgccagcttcggtgtc | | TVKTHPAPGSVVQY | |
| | | ataaccaagttcacagttaaaactcaccctgctcccgg | | TYKISLGSQAQMAP | |
| | | ttctgtcgtacaatacacttacaagatttcgttgggttctc | | VYAAWQALAGDPK | |
| | | aggcccaaatggctccagtttatgcagcttggcaagct | | LDRRFSTLFIAEPLG | |
| | | ttagctggtgacccaaagcttgacagacgtttctctaca | | ALITGTFYGTKAEYE | |
| | | ttgtttatcgctgaaccattgggcgccttaatcaccggc | | ATGIAARLPSGGTLD | |
| | | accttttacggaactaaagctgagtacgaagccacgg | | LKLLDWLGSLAHIA | |
| | | gtattgctgcaagattgccatccggtggtactcttgacc | | EVVGLTLGDIPTSFY | |
| | | taaagcttttggattggttgggttccttggcccacattgc | | GKSLALREEDMLDR | |
| | | tgaagttgtcggtcttactctaggtgacataccaacctc | | TSIDGLFRYMGDAD | |
| | | tttctatggtaagtcattggccttgagagaagaagatat | | AGTLLWFVIFNSEG | |
| | | gctagatagaacctcaatcgatggtttgttcagatacat | | GAMADTPAGATAY | |
| | | gggtgacgctgatgccggtaccttgttatggtttgtcatt | | PHRDKLIMYQSYVI | |
| | | tttaattcggaaggtggtgcaatggcagatacgccag | | GIPTLTKATRDFADG | |
| | | ctggcgcaactgcatatcctcatagagacaaactaatc | | VHDRVRMGAPAAN | |
| | | atgtaccaatcttatgttattggtatcccaactctgacaa | | STYAGYIDRTLSREA | |
| | | aggctaccagggacttcgctgatggtgttcacgacag | | AQEFYWGAQLPRLR | |
| | | agttagaatgggtgctccagctgctaacagtacttacg | | EVKKAWDPKDVFH | |
| | | ctggatacattgatagaaccttatctcgtgaagccgctc | | NPQSVDPAE | |
| | | aagaattttactggggtgcacaattgcctaggttgcgtg | | | |
| | | aggtcaagaaggcttgggacccaaaggatgttttccat | | | |
| | | aatccccaatccgtagacccagctgaa | | | |
| t808154 | Library | atgggcaatactacgtctattgctgccggtagagactg | 89 | MGNTTSIAAGRDCLI | 159 |
| | | tcttatcagcgcagtcggtgctgctaacgtagcctttca | | SAVGAANVAFQDQL | |
| | | agatcagctgctataccaagctacagctgtgcaaccct | | LYQATAVQPYNLNI | |
| | | ataacttaaatatacctgttactccagctgccgttaccta | | PVTPAAVTYPQSAD | |
| | | cccacaaagtgccgacgagatcgctgccgttgtcaaa | | EIAAVVKCASEYGY | |
| | | tgcgcttcggaatatggttacaaggtccaagctaggtc | | KVQARSGGHSFGNY | |
| | | aggtggacactccttcggtaactacggtttgggtggcc | | GLGGQDGAIVIEMK | |
| | | aagatggtgcaattgttattgaaatgaagcatttctctca | | HFSQFSMDESTFIATI | |
| | | gttttctatggacgaatccaccttcatcgctactattggt | | GPGITLGDLDTDLY | |
| | | ccaggaatcaccttgggtgatttggatactgatttatata | | NAGHRAMAHGICPT | |
| | | acgccggtcacagagctatggctcatggtatatgtcca | | IRTGGHLTVGGLGPT | |
| | | accatcagaacgggtggtcacctaacagttggtggttt | | ARQWGLALDHVEE | |
| | | gggccctactgctcgtcaatggggcttagcattggac | | VEVVLANSSIVRASD | |
| | | catgtagaagaagtcgaagttgttctggctaactcttcc | | TQNQDLFFAIKGAA | |
| | | attgtccgtgcttctgacactcaaaatcaggatttgttttt | | ASFGIVTEFKVRTEQ | |
| | | cgctatcaagggtgccgccgcttccttcggtattgtaa | | APGMAVQYSYTFHL | |
| | | cagaatttaaagttagaaccgagcaagctccaggtat | | GTSAEKAKFVKDW | |
| | | ggcagtccaatacagttacaccttccaccttggtacttc | | QAFIAQENLTWKFY | |
| | | agctgaaaaggccaagttcgtcaaagactggcaagc | | TNLVIFDDQIILEGIY | |
| | | cttcattgctcaagaaaacttgacttggaagttttatacc | | FGTKEEYDSLGLEQ | |
| | | aacttggttatattcgatgatcaaatcatcttggaggga | | RFPPTDAGTVLILTD | |
| | | atatactttggtactaaagaagaatacgacagcttaggt | | WLAMIGHGLEDTIL | |
| | | cttgaacaaagattcccaccaactgacgcaggtactgt | | KLVGDTPTWFYAKS | |
| | | gttaattttgacagactggttggcaatgattggtcatgg | | LGFTPRALIPDSAIDE | |
| | | attggaggatacgattttaaagttggttggtgatacacc | | FFDYIHENNPGTLA | |
| | | cacctggttttatgccaagtctctaggtttcaccccaag | | WFVTLSLEGGAINA | |
| | | agctcttattccagatagcgctatcgacgaattttttgac | | VPEDATAYGHRDVL | |
| | | tacatacacgagaataaccctggtactttggcttggttc | | FWFQLFVINPLGPIS | |
| | | gtcacgttatctttggaaggaggtgctatcaacgctgtt | | QTTYGFADGLYDVL | |
| | | ccagaagatgcaaccgcttatggtcacagagatgtctt | | AQAVPESASHAYMG | |
| | | attctggttccaattgttcgttattaatcctttgggtccaat | | CPDPRMPNAQRAY | |
| | | ctcgcagactacttacggtttcgccgacggtctttacga | | WRSNLPKLEELKGY | |
| | | tgtcctggctcaagcagttcccgaatctgcttcgcatgc | | LDPEDIFHNPQGVVP | |
| | | atacatgggttgtccagatccaagaatgccaaacgctc | | S | |
| | | aacgtgcttactggagatccaacttgcctaaactggaa | | | |
| | | gaactaaagggctatttggacccagaagacatttttca | | | |
| | | caatccacaaggtgttgtaccctct | | | |
| t808155 | Library | atgggtaacaccacatcaataactgctggccgtgattg | 90 | MGNTTSITAGRDCL | 160 |
| | | cctgacttccgccgtcggtggagttgctgcacatgtag | | TSAVGGVAAHVAFQ | |
| | | cttttcaagacgccttactatatcagaccccagctgtgg | | DALLYQTPAVDPYN | |
| | | acccttacaatttgaacattccagttacgcccgccgctg | | LNIPVTPAAVTYPQS | |
| | | ttacttacccacaaagcgctgatgaagtcgccgctgtc | | ADEVAAVVKCASD | |
| | | gttaagtgtgcttcggattataattacaaagttcaagcta | | YNYKVQARSGGHSF | |
| | | gatctggtggtcactccttcggtaacttcggtttgggtg | | GNFGLGGQNGAIVV | |
| | | gacaaaatggtgcaatcgtcgttgacatgaagcactttt | | DMKHFSQFSMDEST | |
| | | ctcaattctctatggatgagagtaccttcgtcgccactat | | FVATIGPGTTLGNLD | |
| | | tggtccaggcacaacccttggtaacttggacactgaa | | TEIYNAGKRAMSHG | |
| | | atctacaacgctggtaagagggctatgtctcatggtatt | | ICPSIRTGGHLTVGG | |
| | | tgtcctagtatcagaaccggtggtcacttgactgtagg | | LGPTARQWGLALDH | |
| | | cggtttaggtccaacagctagacaatggggtttggctc | | VEEVEVVLANSSIIR | |
| | | ttgaccacgttgaagaagtcgaagttgtgttggccaac | | ASDTQNQDVLFAIK | |
| | | tcatccattatcagagcttctgatacccagaaccaagat | | GAAASFGIVTEFKVR | |
| | | gtcctatttgcaattaaaggtgctgccgcatccttcgga | | TEEAPGLAVQYSFTF | |
| | | atagtaaccgaatttaaggttagaactgaagaggctcc | | NLGTPAEKAKLVKD | |
| | | aggcttagctgttcaatactccttcactttcaatctgggt | | WQAYIAQENLTWKF | |
| | | acgccagctgaaaaggcaaagttggtgaaagactgg | | YSNLIIFDGQIILEGIF | |
| | | caagcctatatcgcacaggaaaatttgacctggaagtt | | FGSKEEYDQLNLDK | |
| | | ttattctaaccttattatctttgacggtcaaattatcttgga | | KFPTSEPGTVLVLTD | |
| | | gggtattttctttggtagcaaggaagaatacgatcaatt | | WLGMIGHGLEDTIL | |
| | | aaacttagataagaaattccctacttccgaaccaggta | | RLVGDSPTWFYAKS | |
| | | cagttttggtattgactgactggttaggcatgattggtca | | LGFTPSTLISGSAIDG | |
| | | tggtttggaggacaccattctgcgtttagttggtgattct | | LFDYIHKTNAGTLA | |
| | | ccaacatggttttacgctaagtctttgggtttcacacctt | | WFVTLSLEGGAINA | |
| | | ctaccttgatatcaggcagtgctatcgacggtttgttcg | | VPKDATAYGHRDVL | |
| | | attacattcacaaaactaatgcaggaactctagcttggt | | FWVQIFVANPLGPIS | |
| | | ttgttacgttgagtttagaaggtggtgccataaacgctg | | QTTYDFTDGLYDIL | |
| | | tcccaaaggacgctactgcatatggtcatagagatgtc | | AQAVPESAGHAYLG | |
| | | ttgttctgggttcaaatcttcgtcgccaacccacttggtc | | CPDPKMPDAQRAY | |
| | | caatttcgcaaaccacttacgatttcaccgatggtcttta | | WRSNLPRLEELKGD | |
| | | cgacatcctggctcaggctgttcccgaatctgccggtc | | LDPKDIFHNPQGVQ | |
| | | acgcttatttgggttgtcccgatccaaagatgccagac | | VAS | |
| | | gctcaaagagcttattggagatccaatctgcctcgtttg | | | |
| | | gaagaattgaagggtgatctggaccccaaggatatttt | | | |
| | | ccataatccacaaggagttcaagtagcatca | | | |
| t808175 | Library | atgaatccttcaattccatcttcctctatgggcaacacca | 91 | MNPSIPSSSMGNTTS | 161 |
| | | cttccatcgctggtagggattgtctggtcagcgccttag | | IAGRDCLVSALGGN | |
| | | gaggtaacgctggtttggtagcattccagaatcaacca | | AGLVAFQNQPLYQT | |
| | | ctataccaaacaactgctgtgcacgaatataacttgaa | | TAVHEYNLNIPVTPA | |
| | | cataccagtcacccccgccgctattacgtacccagag | | AITYPETAEQIAAVV | |
| | | actgctgaacaaatcgcagctgttgttaaatgcgccag | | KCASQYDYKVQARS | |
| | | tcaatatgactacaaggttcaagctagatcgggtggtc | | GGHSFGNYGLGGTD | |
| | | attcttttggtaattacggtttgggcggtacagacggtg | | GAVVVDMKYFNQF | |
| | | ccgttgtcgttgatatgaagtatttcaaccaattttctatg | | SMDDQTYEAVIGPG | |
| | | gacgatcagacttacgaagctgtcattggtcctggtac | | TTLGDVDVELYNNG | |
| | | cactttaggtgacgtcgatgtagaattgtacaataacg | | KRAMAHGVCPTIST | |
| | | gaaagagagctatggcccacggcgtttgtccaaccat | | GGHFTMGGLGPTAR | |
| | | ctccactggtggtcatttcacgatgggtggtcttggtcc | | QWGLALDHVEEVE | |
| | | aactgctcgtcaatggggtttggctttggatcacgtgg | | VVLANSSIVRASNTQ | |
| | | aggaagttgaagttgtcttagcaaattcatctattgttag | | NQEVFFAVKGAAAS | |
| | | agcaagcaacacacagaaccaagaagtcttctttgct | | FGIVTEFKVRTQPAP | |
| | | gtgaaaggcgctgccgcctcgttcggtatcgttactga | | GIAVQYSYTFNLGSS | |
| | | atttaaggtaagaacccaacccgctccaggaatagct | | AEKAQFIKDWQSFV | |
| | | gttcaatattcttacaccttcaacttaggttcttccgccga | | SAKNLTRQFYTNMV | |
| | | aaaagcccaattcattaaggactggcaatctttcgtatc | | IFDGDIILEGLFFGSK | |
| | | cgctaagaatttgaccagacaattttacacaaatatggt | | EQYEALGLEERFVP | |
| | | tatctttgacggtgatattattttggaaggtcttttcttcgg | | KNPGNILVLTDWLG | |
| | | ttccaaagaacaatatgaggctctgggtttggaagaaa | | MVGHALEDTILRLV | |
| | | gatttgtccctaagaacccaggcaacatcctggtcctg | | GNTPTWFYAKSLGF | |
| | | actgattggctaggtatggttggtcatgcattggaaga | | TPDTLIPSSGIDEFFE | |
| | | caccatactaagattagtcggcaacaccccaacctgg | | YIENNKAGTSTWFV | |
| | | ttttatgctaagtccttgggttttactcctgacactttgatt | | TLSLEGGAINDVPAD | |
| | | ccaagtagcggtatcgatgaatttttcgaatacatagaa | | ATAYGHRDVLFWV | |
| | | aataacaaggctggtacttccacctggttcgttactttg | | QIFMVSPTGPVSSTT | |
| | | agtcttgaaggtggtgctattaacgacgtcccagccga | | YDFADGLYNVLTKA | |
| | | tgctactgcttacggacaccgtgatgttctattctgggta | | VPESEGHAYLGCPD | |
| | | cagatcttcatggtttctcctacaggtccagttagttcta | | PKMANAQQKYWRQ | |
| | | cgacgtatgattttgctgatggtttgtacaatgtgttgac | | NLPRLEELKETLDPK | |
| | | caaagctgttccagaatcagaaggtcacgcttatttag | | DTFHNPQGILPA | |
| | | gatgtccagacccaaagatggccaacgcccaacaaa | | | |
| | | agtattggagacaaaacttgccaagattggaggagtta | | | |
| | | aaggagacattggatcctaaagacactttccataatcc | | | |
| | | ccaaggaatcctaccagcc | | | |
| t808177 | Library | atgggtaacacaaccagtatagccggacgtgattgctt | 92 | MGNTTSIAGRDCLIS | 162 |
| | | gatttcagcacttggtggcaattccgctctagctgttttc | | ALGGNSALAVFPNE | |
| | | ccaaacgagttgctgtggacggctgacgtgcacgaat | | LLWTADVHEYNLNL | |
| | | ataacttaaatttgcccgtaactccagccgctattaccta | | PVTPAAITYPETAAQ | |
| | | ccctgaaactgctgcacaaatcgctggtgttgtcaaat | | IAGVVKCASDYDYK | |
| | | gtgcttctgactacgattataaggttcaggccagatctg | | VQARSGGHSFGNYG | |
| | | gtggtcattcgtttggtaactacggtttgggaggtgcag | | LGGADGAVVVDMK | |
| | | atggcgctgtcgttgtggacatgaagcacttcactcaa | | HFTQFSMDDETYEA | |
| | | ttctcaatggatgacgaaacctacgaagctgttattggt | | VIGPGTTLNDVDIEL | |
| | | ccaggtactacattaaatgacgtcgatatcgaattatat | | YNNGKRAMAHGVC | |
| | | aacaacggtaagagagccatggctcatggtgtctgtc | | PTIKTGGHFTIGGLG | |
| | | caaccatcaaaactggtggtcactttaccatcggtggtt | | PTARQWGLALDHVE | |
| | | tgggtcctactgctaggcaatggggcctagccttggat | | EVEVVLANSSIVRAS | |
| | | catgtcgaagaagttgaagttgttttggctaattcttcca | | NTQNQDVFFAVKGA | |
| | | ttgttagagcttctaacactcaaaatcaagacgtattcttt | | AANFGIVTEFKVRTE | |
| | | gccgtcaagggtgccgctgctaattttggaattgtaac | | PAPGLAVQYSYTFN | |
| | | agagttcaaggtcagaactgaaccagcaccaggttta | | LGSTAEKAQFVKDW | |
| | | gctgttcaatacagctacaccttcaacttgggatccacc | | QSFISAKNLTRQFYN | |
| | | gcagaaaaagctcagttcgtgaaggactggcaatcttt | | NMVIFDGDIILEGLF | |
| | | tatctccgctaaaaaccttacgcgtcaattctataacaa | | FGSKEQYDALGLED | |
| | | catggtcatattcgatggtgatattatattggagggtctg | | HFAPKNPGNILVLTD | |
| | | ttttttggtagtaaagaacaatacgacgctttgggtttgg | | WLGMVGHALEDTIL | |
| | | aagatcacttcgcaccaaagaaccccggcaatatctt | | KLVGNTPTWFYAKS | |
| | | ggttttaactgactggcttggcatggttggtcacgcttta | | LGFRQDTLIPSAGID | |
| | | gaagacacaattttgaagttggtcggtaatactccaac | | EFFEYIANHTAGTPA | |
| | | ctggttctatgccaagtctttaggttttagacaagatact | | WFVTLSLEGGAIND | |
| | | ctaattcctagtgccggaatcgatgaatttttcgaataca | | VAEDATAYAHRDV | |
| | | ttgctaatcatactgctggtactccagcatggttcgttac | | LFWVQLFMVNPLGP | |
| | | gttgtccttagaaggtggtgctataaacgatgtcgccg | | ISDTTYEFTDGLYDV | |
| | | aagatgctactgcctacgctcacagggacgttttgttct | | LARAVPESVGHAYL | |
| | | gggtacaattgtttatggtcaatccattgggtcccatctc | | GCPDPRMEDAQQK | |
| | | tgacaccacgtatgagtttaccgacggtctgtacgatg | | YWRTNLPRLQELKE | |
| | | ttctagctagagctgtgccagaatctgttggtcatgcct | | ELDPKNTFHHPQGV | |
| | | atttgggttgtccagaccctagaatggaagatgcccaa | | MPA | |
| | | cagaagtactggagaaccaaccttccaagattacaag | | | |
| | | aattgaaggaagaactagatccaaagaatacatttcat | | | |
| | | caccctcaaggtgtaatgcctgct | | | |
| t808199 | Library | atgggaaacacaacgtccatagctgccggtagagact | 93 | MGNTTSIAAGRDCL | 163 |
| | | gcctattatcggcagtaggcggtaatcacgctcatgtc | | LSAVGGNHAHVAFQ | |
| | | gctttccaagatcagcttttgtatcaagtgaccgctgttg | | DQLLYQVTAVEPYN | |
| | | agccttacaacttgaatattccagttacccccgccgctg | | LNIPVTPAAVTYPQS | |
| | | ttacttacccacaatcagccgacgaaatcgctgccgtc | | ADEIAAVVKCASEY | |
| | | gtcaaatgtgcttctgaatatggttacaaggttcaagct | | GYKVQARSGGHSFG | |
| | | aggtctggtggtcactcctttggtaactacggtctgggt | | NYGLGGEDGAIVVE | |
| | | ggtgaagatggcgctattgttgtggaaatgaagcattt | | MKHFNQFSMDESTY | |
| | | caatcaatttagtatggatgaatctacttatactgcaact | | TATIGPGITLGDLDT | |
| | | atcggtccaggaattaccttgggtgacttggacaccgc | | ALYNAGHRAMAHG | |
| | | tttatacaacgctggtcacagagccatggcacatggta | | ICPTIRTGGHLTMGG | |
| | | tctgtccaaccatacgtactggtggccacttgaccatg | | LGPTARQWGLALDH | |
| | | ggtggtctgggtcctacagctagacaatggggtttagc | | VEEVEVVLANSSIVR | |
| | | attagatcatgtcgaagaggtcgaagttgttttggctaa | | ASNTQNQDILFAIKG | |
| | | cagctctattgtcagagccagtaacacacagaatcaa | | AAASFGIVTEFKVRT | |
| | | gatattttgttcgctatcaagggtgccgctgcttccttcg | | EAAPGVAVQYSFTF | |
| | | gtattgttactgagtttaaagtaagaactgaagccgctc | | NLGTPAEKAKLVKD | |
| | | caggtgttgcagtccaatactccttcacttttaacctagg | | WQAFIAQEDLTWKF | |
| | | aacgccagctgaaaaggcaaagcttgttaaagactgg | | YSNMNIFDGQIILEGI | |
| | | caagccttcatcgctcaagaagatttgacttggaagttc | | YFGSKEEYDALGLE | |
| | | tattctaacatgaatatatttgacggccaaatcattttgg | | KRFPSSEAGTVLVLT | |
| | | aaggtatctacttcggtagtaaggaagagtacgatgct | | DWLGMVGHGLEDV | |
| | | ttaggtttagaaaagagatttccctcatctgaagctggt | | ILRLVGNTPTWFYA | |
| | | accgtgttggttttgaccgattggttgggtatggtcggc | | KSLGFTPRALIPDSAI | |
| | | cacggtctggaagatgtgattctaagattggttggtaac | | DEFLNYIHENTPGTV | |
| | | accccaacttggttctacgcaaaatcattgggattcact | | SWFVTLSLEGGAIN | |
| | | ccaagagctttgatacctgactcagctattgacgaattt | | KVPGDATAYGHRD | |
| | | cttaattacatccacgaaaacacgcctggtacagtatc | | VLFWVQIFMINPLGP | |
| | | ctggttcgtcactctatctttggaaggtggtgccattaac | | VSQTTYGFADGLYD | |
| | | aaggtcccaggcgatgctactgcctatggccaccgtg | | VLAKAVPNSAGHAY | |
| | | atgtgttattctgggttcagatttttatgatcaacccattg | | LGCPDPRMPNAQQA | |
| | | ggtccagtttctcaaaccacttatggtttcgctgacgga | | YWRSNLPRLEELKG | |
| | | ttatatgacgttttggcaaaggctgtaccaaactcggct | | ELDPKDIFHNPQGV | |
| | | ggacacgcctacttaggttgtcccgatccaagaatgc | | MVVS | |
| | | caaatgctcaacaagcttattggaggtctaatttgccca | | | |
| | | gattggaggaattgaagggtgaactggatccaaaaga | | | |
| | | catttttcataacccacaaggtgttatggttgtctcc | | | |
| t808200 | Library | atgggcaatacgacatccattgcaggtagagattgtct | 94 | MGNTTSIAGRDCLIS | 164 |
| | | tataagcgccctaggtggaaactcggctttggctgcttt | | ALGGNSALAAFPNE | |
| | | ccctaacgagttactgtggactgctgacgtccatgaat | | LLWTADVHEYNLNL | |
| | | acaatttgaacttgcccgttactccagccgctatcacct | | PVTPAAITYPETAEQ | |
| | | atccagaaaccgctgaacaaatcgctggtattgtgaaa | | IAGIVKCASDYDYK | |
| | | tgcgcctctgattacgactataaggttcaggcacgttct | | VQARSGGHSFGNYG | |
| | | ggtggtcactcatttggtaattacggtttgggtggtgcc | | LGGADGAVVVDMK | |
| | | gatggagctgttgtagtcgacatgaagcacttcactca | | HFTQFSMDDETYEA | |
| | | atttagtatggatgacgaaacctacgaagctgtcatcg | | VIGPGTTLNDVDIEL | |
| | | gtccaggtacaactttaaacgacgttgatattgaattata | | YNNGKRAMAHGVC | |
| | | taacaatggcaaaagagccatggcacatggtgtttgtc | | PTIKTGGHFTIGGLG | |
| | | caactatcaagaccggaggtcacttcaccattggtggt | | PTARQWGLALDHVE | |
| | | ttgggtcctacagctagacaatggggtttggctctgga | | EVEVVLANSSIVRAS | |
| | | ccacgtcgaggaagtagaagttgtcttggcaaactctt | | NTQNQDVFFAVKGA | |
| | | ccattgtgagggcctctaacactcaaaatcaagatgttt | | AANFGIVTEFKVRTE | |
| | | tctttgcagttaagggtgctgctgctaacttcggtatagt | | PAPGLAVQYSYTFN | |
| | | gaccgagtttaaagttagaacggaaccagctccaggc | | LGSTAEKAQFVKDW | |
| | | ttagctgtccagtactcctatactttcaacttgggttcaa | | QSFISAKNLTRQFYN | |
| | | ctgctgaaaaggctcaattcgttaaggattggcaatcat | | NMVIFDGDIILEGLF | |
| | | tcatctctgctaagaatcttactagacaattttacaacaa | | FGSKEQYDALGLED | |
| | | catggtcatttttgacggtgatatcattttagaaggtttatt | | HFAPKNPGNILVLTD | |
| | | tttcggcagtaaggaacaatacgacgccttgggtttgg | | WLGMVGHALEDTIL | |
| | | aagatcattttgcaccaaagaaccctggtaacattttgg | | KLVGNTPTWFYAKS | |
| | | tactaaccgactggttgggaatggttggtcacgcccta | | LGFRQDTLIPSAGID | |
| | | gaagatacaatattgaaattggttggcaatactccaac | | EFFEYIANHTAGTPA | |
| | | ctggttctacgctaaatctttgggtttcagacaggatac | | WFVTLSLEGGAINDI | |
| | | cttgattccatccgctggtatcgacgaatttttcgaatat | | AEDATAYAHRDVLF | |
| | | attgctaatcatactgctggtaccccagcttggttcgtc | | WVQLFMVNPLGPIS | |
| | | accttaagcctagagggtggtgccatcaatgatatcgc | | DTTYEFTDGLYDVL | |
| | | tgaagacgctactgcctacgcacatagagatgtcttatt | | ARAVPESVGHAYLG | |
| | | ctgggtccaactgtttatggttaaccctttgggtcccata | | CPDPRMEDAQQKY | |
| | | tctgatacaacttacgaatttacagacggtctgtatgac | | WRTNLPRLQELKEE | |
| | | gttctagcacgtgctgtaccagagtctgtcggccacgc | | LDPKNTFHHPQGVM | |
| | | ttacttaggctgtcccgacccaagaatggaagacgca | | PA | |
| | | caacaaaagtattggagaaccaacctaccaagattgc | | | |
| | | aagaattgaaggaagagttggacccaaagaacacgtt | | | |
| | | tcaccatccacagggtgttatgcctgca | | | |
| t808223 | Library | atgggtaatacgacttccatagccggaagggactgcc | 95 | MGNTTSIAGRDCLIS | 165 |
| | | taatctctgctttgggtggtaactcggctctggcagtctt | | ALGGNSALAVFPNE | |
| | | ccctaacgagttattgtggaccgctgatgttcacgaata | | LLWTADVHEYNLNL | |
| | | caatttgaacttgccagttactccagccgctattacctat | | PVTPAAITYPETAAQ | |
| | | cccgaaacagctgcacagattgctggcgtagtcaaat | | IAGVVKCASDYDYK | |
| | | gtgcctcagattacgactacaaggtgcaagctagatct | | VQARSGGHSFGNYG | |
| | | ggtggtcatagctttggtaactatggtttaggaggtgct | | LGGADGAVVVDMK | |
| | | gatggcgcagttgttgtcgacatgaagcacttcactca | | HFTQFSMDDETYEA | |
| | | atttagtatggatgacgaaacttacgaagctgttatcgg | | VIGPGTTLNDVDIEL | |
| | | tccaggtaccaccctaaatgatgttgatatcgaattgtat | | YNNGKRAMAHGVC | |
| | | aacaatggtaagagagctatggcacatggtgtttgtcc | | PTIKTGGHFTIGGLG | |
| | | aacaattaaaactggaggtcacttcaccattggcggttt | | PTARQWGLALDHVE | |
| | | aggtcctactgccagacaatggggtcttgctttggacc | | EVEVVLANSSIVRAS | |
| | | atgtcgaagaagtagaggtcgttcttgctaactcttctat | | NTQNQDVFFAVKGA | |
| | | cgttcgtgcttccaacactcaaaaccaagatgtgttcttt | | AANFGIVTEFKVRTE | |
| | | gccgtcaagggtgctgctgccaacttcggtattgtaac | | PAPGLAVQYSYTFN | |
| | | agaatttaaagttagaactgaaccagctccaggtttag | | LGSTAEKAQFVKDW | |
| | | ccgtccagtactcttataccttcaatttgggttccacggc | | QSFISAKNLTRQFYN | |
| | | tgaaaaggctcaattcgttaaggactggcaatccttcat | | NMVIFDGDIILEGLF | |
| | | atctgccaagaatttgaccagacaattttacaataacat | | FGSKEQYDALGLED | |
| | | ggttatctttgacggagatattatattggagggtctatttt | | HFAPKNPGNILVLTD | |
| | | tcggtagtaaggaacaatacgacgctctgggcttaga | | WLGMVGHALEDTIL | |
| | | agatcactttgctccaaaaaacccaggtaatatcttggt | | KLVGNTPTWFYAKS | |
| | | attgaccgattggttgggtatggtcggtcatgcccttga | | LGFRQDTLIPSAGID | |
| | | agatacaattttgaagctggttggtaacactccaacttg | | EFFEYIANHTAGTPA | |
| | | gttctacgcaaagtccttaggtttccgtcaagacacgtt | | WFVTLSLEGGAIND | |
| | | aattccttcagccggcatcgatgaatttttcgaatacatc | | VAEDATAYAHRDV | |
| | | gctaaccacaccgctggtactcctgcttggttcgtcac | | LFWVQLFMVNPVGP | |
| | | cttgagcttggaaggcggtgccattaacgatgtcgcc | | ISDTTYEFTDGLYDV | |
| | | gaggacgcaacggcttacgctcacagagatgttttgtt | | LARAVPESVGHAYL | |
| | | ctgggtccaattattcatggtgaatccagtgggtcctat | | GCPDPRMEDAQQK | |
| | | atctgacactacttatgaatttactgatggtttgtacgac | | YWRTNLPRLQELKE | |
| | | gttctagctagagcagtccctgagagcgtgggtcatg | | ELDPKNTFHHPQGV | |
| | | cttatttgggttgtccagacccaagaatggaagatgcc | | MPA | |
| | | caacagaaatattggaggacaaatttacccagattgca | | | |
| | | agaattaaaagaggaattggatccaaagaacacattc | | | |
| | | caccatccacagggtgttatgcccgct | | | |
| t808225 | Library | atgggcaatacaacgtccattgccgctggtcgtgactg | 96 | MGNTTSIAAGRDCLI | 166 |
| | | cttgatcagcgctgttggaggtaacgcagctcacgtg | | SAVGGNAAHVAFQ | |
| | | gcctttcaggatcaacttttatatcaagctaccgcagtc | | DQLLYQATAVDVY | |
| | | gatgtttacaacttgaacatacccgtcactccagctgcc | | NLNIPVTPAAVTYPQ | |
| | | gtaacttaccctcaatcagctgacgaggttgctgctgtt | | SADEVAAVVKCASE | |
| | | gtcaagtgtgcctcggaatacgattataaagtccaagc | | YDYKVQARSGGHSF | |
| | | tagatctggtggtcattctttcggtaattacggtctaggt | | GNYGLGGQNGAIVV | |
| | | ggtcaaaatggagctattgttgtcgacatgaagcactt | | DMKHFSQFSMDEST | |
| | | cagtcaatttagtatggacgaatcaacctatactgcaac | | YTATIGPGITLGDLD | |
| | | catcggcccaggtatcactctgggtgatttagataccg | | TELYNAGHRAMAH | |
| | | aattgtacaacgctggtcatagagcaatggctcacggt | | GICPTIRTGGHLTIGG | |
| | | atttgtccaacaataagaactggtggtcacttgactatc | | LGPTARQWGLALDH | |
| | | ggtggtttgggtccaacagccaggcagtggggtctg | | VEEVEVVLANSSIVR | |
| | | gctttagaccatgttgaagaggtagaagttgtgttggct | | ASETQNQDVLFAVK | |
| | | aactcttccattgttagagcctctgaaacgcaaaacca | | GAAASFGIVTEFKVR | |
| | | agatgtcttgttcgcagtaaagggcgctgctgcttcctt | | TEQAPGLAVQYSYT | |
| | | tggtattgttaccgaatttaaagttagaactgaacaagc | | FNLGTPAEKAKLLK | |
| | | tcctggcctagctgtccagtattcctacaccttcaatttg | | DWQAFIAQEDLTWK | |
| | | ggtaccccagctgagaaggccaagttattaaaagact | | FYSNMVIFDGQIILE | |
| | | ggcaagctttcatcgcccaagaagacttgacctggaa | | GIFFGSKEEYDALDL | |
| | | gttctactccaatatggttattttcgatggtcaaatcatttt | | EKRFPTSEPGTLLVL | |
| | | ggaaggaattttctttggttctaaggaagaatatgatgc | | TDWLGMVGHSLED | |
| | | cctggatcttgagaagagatttccaacttctgaacctgg | | VILRLVGNTPTWFY | |
| | | tactttgttggttttaacggactggcttggtatggtaggt | | AKSLGFTPRTLIPDS | |
| | | catagcctggaagacgtcatattaaggctagttggtaa | | AIDRFFDYIHETNAG | |
| | | caccccaacttggttttacgctaagtctttgggcttcact | | TLAWFVTLSLEGGAI | |
| | | ccaagaaccttgatccctgacagcgctatagatagatt | | NAVPEDATAYGHRD | |
| | | cttcgactatattcacgaaactaacgctggtaccttggc | | VLFWVQIFMVNPLG | |
| | | atggtttgtgacgctttcattggaaggtggtgctattaat | | PISQTIYDFADGLYD | |
| | | gccgtgccagaagatgcaaccgcctacggtcatcgt | | VLAQAVPESAEHAY | |
| | | gatgttttgttttgggttcaaatcttcatggtcaacccctt | | LGCPDPKMPDAQRA | |
| | | gggaccaatttctcaaactatctacgatttcgctgacgg | | YWRGNLPRLEELKG | |
| | | actatacgacgtgttggcacaagccgtaccagaatcg | | EFDPKDTFHNPQGV | |
| | | gctgaacacgcttacttaggatgtccagatcctaaaat | | SVAV | |
| | | gccagacgcccaacgtgcttattggagaggtaactta | | | |
| | | ccaagactggaggaattgaaaggagagtttgatccca | | | |
| | | aggacacatttcacaacccacagggtgtttctgtcgcc | | | |
| | | gtc | | | |
| t808226 | Library | atgggcaacaccacgagcatcgctgccggtagagat | 97 | MGNTTSIAAGRDCLI | 167 |
| | | tgtttaatatctgctgttggaggtaatgcagctcacgtc | | SAVGGNAAHVAFQ | |
| | | gcctttcaggaccaactgctttaccaagctactgctgtg | | DQLLYQATAVEPYN | |
| | | gaaccttataacctaaatattccaatcaccccagccgct | | LNIPITPAAITYPQSA | |
| | | attacatacccccaatcggctgatgagatcgcagcagt | | DEIAAVVKCASEYG | |
| | | tgtaaagtgcgcttcagaatatggttacaaagtccaag | | YKVQARSGGHSFGN | |
| | | ctcgttccggtggtcattctttcggtaactacggtttagg | | YGLGGEDGAIVVEM | |
| | | tggtgaagacggtgctattgttgtcgaaatgaagcactt | | KHFSQFSMDESTYIA | |
| | | cagtcaattttccatggatgaatctacttatattgccacta | | TIGPGITLGDLDTEL | |
| | | tcggcccaggtattacattgggagacttggataccgaa | | YNVGHRAMAHGICP | |
| | | ttatacaatgttggtcatagagctatggcccacggtatc | | TIRTGGHLTVGGLGP | |
| | | tgtccaactattagaaccggtggtcatttgactgttgga | | TARQWGLALDHVE | |
| | | ggtttgggtcctaccgctaggcaatggggcctggcctt | | EVEVVLANSSIVRAS | |
| | | ggatcacgttgaggaagtcgaagtcgtattggctaact | | DTQNQDIFFAIKGAA | |
| | | cttccatagttagagcatcagacactcagaaccaaga | | ASFGIVTEFKVRTEQ | |
| | | catcttcttcgctattaaaggtgctgctgctagctttggt | | APGLAVQYSYTFNL | |
| | | atagtgacagaatttaaggttagaaccgagcaagccc | | GTPAEKAKLVKDW | |
| | | caggtctagccgtgcaatactcttacactttcaacttgg | | QAFIAQENLSWKFY | |
| | | gtacaccagctgaaaaggccaagttggttaaggactg | | SNMVVFDGQIILEGL | |
| | | gcaggctttcattgctcaagaaaatctgtcatggaaatt | | YFGSKEEYDALGLE | |
| | | ctactctaatatggtcgtattcgatggccaaatcatctta | | QRFPPSEAGNVLVLT | |
| | | gaaggtttgtactttggctccaaggaagaatatgatgct | | DWLGMVGHELEDTI | |
| | | cttggtcttgaacaacgtttccccccatctgaagctggt | | LRLVGNTPTWFYAK | |
| | | aacgttctagtcttgactgattggttgggtatggttggtc | | SLGFTPRALIPDSAID | |
| | | atgagttagaagatactattttgagattggtaggtaaca | | DLFNYIHENNPGTLA | |
| | | cccctacttggttctacgctaaaagcttgggatttaccc | | WFVTLSLEGGAINT | |
| | | caagagccctgattccagactccgcaatagatgactta | | VPEHATAYGHRDVL | |
| | | ttcaactatattcacgagaataacccaggtaccttggca | | FWVQIFVINPLGPVS | |
| | | tggttcgtcacactttctttagaaggtggtgcaatcaac | | QTTYGFADGMYDV | |
| | | accgttcctgaacacgctactgcctatggacatagaga | | LAQAVPESAGHAYL | |
| | | tgttttgttttgggtccaaatttttgttatcaatccattgggt | | GCPDPRMPNAQQAY | |
| | | cccgtcagccaaacgacttacggttttgctgatggtat | | WRSNLPRLEELKGD | |
| | | gtatgacgtgcttgcccaagctgttccagaaagtgctg | | LDPKGIFHNPQGVM | |
| | | gtcatgcttacttgggttgtccagatccacgtatgccaa | | VVS | |
| | | acgcccaacaagcttactggagatctaatttgcctaga | | | |
| | | ttagaagaattgaagggcgacctagacccaaaaggta | | | |
| | | tcttccacaatccacaaggtgttatggtagtctcc | | | |
| t808232 | Library | atgggtaacactacgtcgatcgcagctggacgtgatt | 98 | MGNTTSIAAGRDCL | 168 |
| | | gcctattgtccgctgttggtggcaatcatgcccacgta | | LSAVGGNHAHVAFQ | |
| | | gctttccaggaccaacttttgtatcaagccacagctgtc | | DQLLYQATAVEPYN | |
| | | gaaccatacaacttaaacatacctgtgactccagctgc | | LNIPVTPAAVTYPQS | |
| | | cgttacctacccccaatctgctgatgaggtcgcagctg | | ADEVAAVVKCAAD | |
| | | ttgttaagtgtgctgccgactatggttacaaagtccaag | | YGYKVQARSGGHSF | |
| | | ctagatcaggtggtcacagttttggtaattacggtttgg | | GNYGLGGEDGAIVV | |
| | | gtggtgaagacggtgctattgttgtagatatgaagcatt | | DMKHFDQFSMDEST | |
| | | tcgatcaatttagcatggatgaatctacctacactgcca | | YTATIGPGITLGDLD | |
| | | ccatcggcccaggtattactctgggcgacttggatacc | | TALYNAGHRAMAH | |
| | | gctttatataatgccggtcacagagctatggcacatgg | | GICPTIRTGGHLTIGG | |
| | | tatctgtccaactattagaacaggcggtcacttgaccat | | LGPTARQWGLALDH | |
| | | tggtggtttgggtcctacggctaggcaatggggattgg | | VEEVEVVLANSSIVR | |
| | | cactagaccacgtcgaagaagttgaggttgtcctggct | | ASDTQNQEILFAVK | |
| | | aactcctctatagtcagagcctctgacactcagaacca | | GAAASFGIVTEFKVR | |
| | | agaaattttattcgctgttaagggtgctgccgcttccttc | | TEEAPGLAVQYSFTF | |
| | | ggtatcgtcactgaatttaaagttagaaccgaagaagc | | NLGTAAEKAKLVKD | |
| | | tccaggattggcagtccaatacagcttcaccttcaacct | | WQAFIAQEDLTWKF | |
| | | tggtactgccgctgaaaaggctaagttggtgaaagatt | | YSNMNIIDGQIILEGI | |
| | | ggcaagcttttatcgcccaggaagacttaacgtggaa | | YFGSKAEYDALGLE | |
| | | gttttattctaacatgaacattatcgatggtcaaattattct | | EKFPTSEPGTVLVLT | |
| | | ggagggtatctacttcggttcgaaagctgaatacgac | | DWLGMVGHGLEDV | |
| | | gcattgggattggaagagaagtttccaacatcagaac | | ILRLVGNAPTWFYA | |
| | | ccggtactgtgcttgtattaactgactggttgggtatggt | | KSLGFAPRALIPDSAI | |
| | | tggtcacggtttagaagatgttattttgcgtttggttgga | | DDFFEYIHKNNPGT | |
| | | aatgctccaacttggttttatgcaaagtcactaggtttcg | | VSWFVTLSLEGGAI | |
| | | ctccaagagctttaatacctgatagtgcaattgatgactt | | NKVPEDATAYGHRD | |
| | | cttcgaatatatccataagaataacccaggtacagtctc | | VLFWVQIFMINPLGP | |
| | | ttggttcgtcaccttgtccttggagggtggtgccatcaa | | VSQTIYDFADGLYD | |
| | | taaagtaccagaagatgccactgcttacggtcataga | | VLAKAVPESAGHAY | |
| | | gatgttctattctgggttcaaatttttatgatcaatccatta | | LGCPDPRMPNAQQA | |
| | | ggtccagtttctcaaacgatctacgatttcgctgacggc | | YWRNNLPRLEELKG | |
| | | ttgtatgacgttctggctaaggccgtacctgaatccgct | | DLDPKDIFHNPQGV | |
| | | ggtcacgcatacctaggttgtcccgacccaagaatgc | | MVVS | |
| | | ctaacgctcaacaggcctactggaggaacaacttgcc | | | |
| | | aagattggaagaattgaagggtgacttagatccaaaa | | | |
| | | gatattttccataatcctcaaggagtgatggtcgtgagc | | | |
| t808237 | Library | atgggtaatacgacttccatcgccggccgtgactgctt | 99 | MGNTTSIAGRDCLV | 169 |
| | | ggttagtgcactaggtggaaacgctggtttagtggcttt | | SALGGNAGLVAFQD | |
| | | ccaagatcagcttttgtatcaaaccacagctgtacacg | | QLLYQTTAVHEYNL | |
| | | agtacaacttgaacattccagtcacccctgccgcagtt | | NIPVTPAAVTYPETA | |
| | | acttacccagaaactgctgaacaaatagctgccgtcgt | | EQIAAVVKCASEYD | |
| | | gaaatgtgcttctgaatatgattacaaggtccaagctag | | YKVQARSGGHSFGN | |
| | | atctggtggacattcgtttggtaattacggtctaggtggt | | YGLGGADGAVVVD | |
| | | gctgacggtgctgtagttgttgatatgaagcacttctca | | MKHFSQFSMDDQTY | |
| | | caattttccatggacgatcagacatatgaagcagttatc | | EAVIGPGTTLGDVD | |
| | | ggtcccggtaccactttaggtgacgtcgacaccgaatt | | TELYNNGKRAMAH | |
| | | gtacaacaacggcaagagagctatggcccatggtatt | | GICPTISTGGHFTMG | |
| | | tgtccaacaattagtactggtggacacttcactatgggt | | GLGPTARQWGLALD | |
| | | ggtctgggtccaaccgccagacaatggggtttggcttt | | HVEEVEVVLANSSIV | |
| | | ggatcacgttgaagaggttgaagtcgttttggcaaattc | | RASNTQNQEVFFAV | |
| | | ttctatcgttagggcttccaacacccaaaatcaagaagt | | KGAAASFGIVTEFK | |
| | | cttctttgctgtcaaaggtgccgctgcctcatttggtatc | | VRTQPAPGLAVQYS | |
| | | gttacagagttcaaggtcagaactcaacctgctccag | | YTFNIGSSAEKAQFV | |
| | | gcttagcagtacagtacagctatacgtttaatattggttc | | KDWQSFISAKNLTR | |
| | | gtctgctgaaaaggcccaattcgttaaagattggcaat | | QFYTNMVIFDGDIIL | |
| | | cattcattagtgctaagaaccttactagacaattctacac | | EGLFFGSQEQYEAL | |
| | | caacatggtaatcttcgatggtgacataattttggaagg | | GLEDRFVPKNPGNIL | |
| | | attatttttcggttcccaagaacaatatgaagctttgggt | | VLTDWLGMVGHAL | |
| | | ctggaagacagatttgttccaaagaaccctggaaatat | | EDTILRLVGNTPTWF | |
| | | tttggtgttgacggattggctgggtatggttggtcatgc | | YAKSLGFTPDTLIPA | |
| | | ccttgaagacactatcttaagattggtcggtaacactcc | | SGIDEFFDYIENHKA | |
| | | aacttggttttacgctaaatctttgggattcaccccagac | | GTLTWFVTLSLEGG | |
| | | actttaattccagcttccggtatcgatgaatttttcgatta | | AINDVPEDATAYGH | |
| | | catagaaaaccataaggcaggcaccttgacgtggttc | | RDVLFWVQIFMASP | |
| | | gtcactttgtctctggaaggtggtgctatcaatgatgtc | | TGPVSSTTYDFADG | |
| | | ccagaggacgctacagcctacggtcatagagatgtttt | | LYNVLTKAVPESEG | |
| | | gttctgggttcaaatttttatggcttctcccaccggtcct | | HAYLGCPDPKMAD | |
| | | gtctcctctaccacctatgacttcgccgatggtctatata | | AQQKYWRQNLPRL | |
| | | atgttttaactaaggctgtaccagagagcgaaggtcac | | EELKATLDPKDTFH | |
| | | gcttacttaggttgtccagaccctaagatggccgatgc | | NPQGILPA | |
| | | tcagcaaaaatactggcgtcaaaacttgccaagattgg | | | |
| | | aagaattgaaggcaactttagacccaaaagataccttc | | | |
| | | cacaatccccaaggtatcttgccagct | | | |
| t808238 | Library | atgtggttgtctacaatgaatggttcagccagtagacgt | 100 | MWLSTMNGSASRRS | 170 |
| | | agcgatcccgtcagcagaaaaatcgtttgcgacggcc | | DPVSRKIVCDGHAS | |
| | | atgcttctgcacacgaggtgaggactgacaacgaag | | AHEVRTDNEAARDV | |
| | | ctgctagagatgtaccttcgagaaccgctgtcaacaa | | PSRTAVNKERKQGS | |
| | | ggaaagaaagcagggttccggtccaccaggagccat | | GPPGAMQRGFHAA | |
| | | gcaaagaggttttcacgctgcccataagccaaatgaa | | HKPNEMVPQDGPLG | |
| | | atggttccacaagacggtcctcttggtagaactgctca | | RTAQLFRLAPACQS | |
| | | attattccgtctggcaccagcttgtcaatcagaaccaac | | EPTRAPGQPSDLRLR | |
| | | gagagctccaggtcaaccatctgatctaagattgcgtc | | QIPLATEQAARTLAR | |
| | | aaattcccttggcaaccgagcaagctgccagaactttg | | MRPARFTFPYGRAA | |
| | | gctaggatgcgtccagcaagattcacatttccttatgga | | EDDCYLKKEDEGHD | |
| | | agagccgctgaagatgattgttacttaaaaaaggaag | | QSHPTSVLVGVPPFT | |
| | | acgaaggtcacgaccagtctcatccaacctccgtcttg | | RRCAAAETFKDTRA | |
| | | gttggtgttccccctttcactagacgttgtgctgctgctg | | RAPGTQPTDTTSTG | |
| | | aaaccttcaaagatactagagctagagctccaggtac | | ASPSWTLSPLLSLSA | |
| | | acaaccaactgataccacttctacaggtgcctccccat | | TDDSVPSKMGNGQS | |
| | | catggaccttatctccactattgtccttgtctgctactga | | TPLQQCLNTVCNGR | |
| | | cgacagtgttccttccaagatgggcaacggtcaatcta | | LGCVAFPSDALYQA | |
| | | ccccacttcaacaatgtttgaatactgtttgcaacggta | | AWVKPYNLDVPVTP | |
| | | ggctgggttgtgtcgcctttccatcggatgccttatacc | | IAVFKPSSTEDVAGA | |
| | | aggctgcttgggtcaagccatataacttggacgtacct | | IKCAVASNVHVQAK | |
| | | gttaccccaatagctgtgtttaagcctagttccacggag | | SGGHSYANFGLGGQ | |
| | | gatgttgctggtgccattaagtgtgctgtcgcttctaac | | DGELMIDLANLQDF | |
| | | gttcacgtgcaagccaagtctggtggtcattcgtacgc | | HMDKTSWQATFGA | |
| | | taacttcggattgggtggacaagatggtgaactaatga | | GYRLGDLDKKLQA | |
| | | ttgatttggcaaatttacaggacttccacatggacaaaa | | NGNRAIAHGTCPGV | |
| | | catcctggcaagctactttcggtgccggttacagattg | | GIGGHATIGGLGPMS | |
| | | ggtgatttagataaaaagttacaagcaaatggcaaca | | RMWGSALDHVLSV | |
| | | gagctatcgcacacggcacatgtccaggtgttggtatt | | QVVTADGSIKNASE | |
| | | ggaggtcatgccactatcggcggtctaggcccaatga | | SENSDLFWALRGAG | |
| | | gccgtatgtggggttccgctttggaccacgtcttgtctg | | ASFGVITKFTVKTHP | |
| | | tccaagttgtcaccgctgacggtagtatcaaaaacgcc | | APGSVVQYTYKISL | |
| | | tctgaatcagaaaactctgatctgttttgggccttgaga | | GSQAQMAPVYAAW | |
| | | ggtgctggtgcttcatttggagttattactaagttcactgt | | QALAGDPKLDRRFS | |
| | | taagacccatccagctccaggttccgttgtacaatatac | | TLFIAEPLGALITGTF | |
| | | atacaaaatctctttgggtagccaggcacaaatggccc | | YGTKAEYEATGIAA | |
| | | ctgtttacgctgcctggcaagctttagctggtgacccc | | RLPSGGTLDLKLLD | |
| | | aagctggacagaagattttccacattgttcatcgcaga | | WLGSLAHIAEVVGL | |
| | | acctcttggtgctctgattaccggaactttctatggtact | | TLGDIPTSFYGKSLA | |
| | | aaagctgagtacgaagctactggtatagctgctagatt | | LREEDMLDRTSIDGL | |
| | | gccttctggtggtaccttggatttgaagcttttagattgg | | FRYMGDADAGTLL | |
| | | ttgggtagtttggctcacatagctgaagtagttggtttga | | WFVIFNSEGGAMAD | |
| | | ccttgggtgacattccaacgtccttttacggtaagtcgc | | TPAGATAYPHRDKL | |
| | | tagctttgagagaagaagacatgttagaccgtacttcta | | IMYQSYVIGIPTLTK | |
| | | ttgatggtttgttcaggtatatgggtgacgccgatgcag | | ATRDFADGVHDRVR | |
| | | gcaccctattatggttcgtcatcttcaattccgagggtg | | MGAPSANSTYAGYI | |
| | | gtgccatggctgatactccagctggtgccaccgccta | | DRTLSREAAQEFYW | |
| | | cccacatagagataaattaatcatgtatcaatcatacgtt | | GAQLPRLREVKKA | |
| | | attggtataccaaccctgactaaggccaccagagattt | | WDPKDVFHNPQSVD | |
| | | tgctgatggtgtccacgaccgtgtgagaatgggtgctc | | PAE | |
| | | catctgctaacagtacgtatgcaggttacatcgataga | | | |
| | | accttgtccagagaagctgctcaagaattttactgggg | | | |
| | | cgctcaactgcctagattgagagaagtcaagaaagca | | | |
| | | tgggacccaaaggacgtctttcacaacccacaatccg | | | |
| | | ttgatccagcagag | | | |
| t808240 | Library | atgggcaatacaacttccattggtgtagtgagggattgt | 101 | MGNTTSIGVVRDCL | 171 |
| | | ttgacgtctgctgtcggtggtgttgcagcccatgtcgct | | TSAVGGVAAHVAFQ | |
| | | ttccaggacaccctattataccaaacctcagctgttaaa | | DTLLYQTSAVKPYN | |
| | | ccatataaccttaacgtccctgttactcccgccgctgtt | | LNVPVTPAAVTYPQ | |
| | | acttacccacaaagtgctaatgaagtcgctgctatcgtt | | SANEVAAIVKCASD | |
| | | aagtgcgcatcggattatgactacaaggtacaagctc | | YDYKVQARSGGHSF | |
| | | gttccggtggacacagctttggtaacttcggtttaggtg | | GNFGLGGQNGAIVI | |
| | | gacaaaacggtgccatagttattgacatgaaacactttt | | DMKHFSQFSMDEST | |
| | | ctcaattctccatggatgagtctaccttcatcgccactat | | FIATIGPGTTLGNLD | |
| | | tggcccaggcaccactttgggtaatctggatacagaat | | TELYNAGNRAMAH | |
| | | tgtacaacgctggtaatagagctatggctcatggtatat | | GICPSIRTGGHLTVG | |
| | | gtccatcgatcagaactggtggtcacttgaccgttgga | | GLGPTSRQWGLALD | |
| | | ggtttgggtcctacctctcgtcaatggggtctagctctg | | HVEEVEVVLANSSV | |
| | | gaccacgtcgaagaggtggaagttgtacttgctaactc | | VRASDTQNQDVLFA | |
| | | ttcagtcgtcagagcctctgacacgcagaaccaagat | | IKGAAASFGIVTEFK | |
| | | gttttatttgctatcaagggtgcagccgcatccttcggta | | VRTEEAPGLAVRYS | |
| | | tcgttactgaatttaaggtcagaacagaagaagctcca | | YSFNLGTPAEKAKL | |
| | | ggtttggccgttagatattcctacagcttcaacttgggta | | AKDWQAYIAQENLT | |
| | | ctccagctgaaaaagcaaagttggctaaggattggca | | WKFSSNLIIFDGQIIL | |
| | | agcctacattgcccaagaaaacttaacgtggaaattct | | EGIFFGSKEEYDKLN | |
| | | ctagtaacttgattattttcgacggtcaaattatccttgag | | LEKKFPTSEPGTVLV | |
| | | ggaatatttttcggtagcaaggaagaatacgacaagtt | | ITNWLGMIGHALED | |
| | | aaatttggaaaagaagtttccaacttcagaacctggta | | TILRLIGDSPTWFYA | |
| | | ccgtcttggtcattacgaattggttgggtatgatcggac | | KSLGFTPNTLIFDSTI | |
| | | atgctttggaagataccatcctaagacttatcggtgatt | | DEFFDYIHKANAGT | |
| | | cacccacttggttctatgctaaatctttgggttttactcca | | LAWSVMLSLEGGAI | |
| | | aacacactaatctttgactctaccattgacgaatttttcg | | NAVPKNATAYGHR | |
| | | attacatacacaaggctaacgctggtacattagcttggt | | DVLFWVQIFVVNPL | |
| | | ccgttatgttgtctttggaaggtggtgccataaatgctgt | | GPISQTTYGFTDGLY | |
| | | tccaaaaaatgctactgcatacggtcatagagatgtatt | | NILARGVPESAGHA | |
| | | attctgggttcaaattttcgttgtgaatcctcttggaccaa | | YLGCPDPKMPDAQR | |
| | | tttcccaaaccacttatggttttaccgatggtttgtataac | | AYWRNNYPRLEELK | |
| | | atcttggccagaggtgttccagagtccgcaggtcatg | | RDLDPKDIFHNPQG | |
| | | cttacttaggttgtccagatcccaagatgccagacgct | | VRVAS | |
| | | caaagagcatactggagaaataactatccacgtctgg | | | |
| | | aggaattgaaaagagacttggatcctaaggacatttttc | | | |
| | | acaacccacagggcgtcagagtcgcttct | | | |
| t808247 | Library | atgggcaacactacatcaattgctgccggtagagattg | 102 | MGNTTSIAAGRDCL | 172 |
| | | cctagtaagcgcagtcggtccagctcatgttaccttcc | | VSAVGPAHVTFQDA | |
| | | aggacgcccttctgtaccaaactacggctgtcgatcct | | LLYQTTAVDPYNLN | |
| | | tataatttaaacatcccagtgacccccgctgctgttactt | | IPVTPAAVTYPQSAE | |
| | | acccacaatcggctgaagagatagccgctgttgtcaa | | EIAAVVKCASDYDY | |
| | | atgtgcttctgactatgattacaaggttcaagctaggtct | | KVQARSGGHSFGNY | |
| | | ggtggacactcctttggtaactacggtttgggtggtca | | GLGGQNGAIVVDM | |
| | | aaatggagccattgtagttgacatgaagcacttctctca | | KHFSQFSMDESTFV | |
| | | atttagtatggatgaatctaccttcgtcgcaactattggt | | ATIGPGTTLGDLDTE | |
| | | ccaggtacaaccttgggcgacttggatactgaattgta | | LYNAGGRAMAHGIC | |
| | | taacgcaggcggtagagctatggcccatggtatctgt | | PTIRTGGHLTVGGLG | |
| | | cctacaatccgtactggtggtcacttaactgtcggtggt | | PTARQWGLALDHIE | |
| | | ttgggtccaaccgctagacaatggggtctggccttag | | EVEVVLANSSIVRAS | |
| | | atcacattgaagaagttgaagtggttttggctaattcctc | | NTQNQDILFAVKGA | |
| | | gatagtgagagctagcaacactcagaaccaagacat | | AASFGIVTEFKVRTQ | |
| | | cttgttcgccgttaagggtgctgctgcttcatttggtatt | | EAPGLAVQYSFTFN | |
| | | gtcaccgagtttaaagttagaacccaagaagcaccag | | LGSPAQKAKLVKD | |
| | | gactagctgttcaatacagtttcaccttcaatttgggttc | | WQAFIAQENLSWKF | |
| | | cccagctcagaaagccaagttggtcaaggactggca | | YSNLVIFDGQIILEGI | |
| | | agcattcattgcccaagaaaacttatcttggaagttcta | | FFGSKEEYDELDLEK | |
| | | ctctaatttagtcatctttgacggtcaaattattttagaag | | RFPTSEPGTVLVLTD | |
| | | gtatctttttcggatccaaggaggaatatgatgaattgg | | WLGMIGHALEDTIL | |
| | | acttggaaaaaagatttcccacttctgaaccaggtaca | | KLVGDTPTWFYAKS | |
| | | gttctggttttaacggattggttgggaatgatcggccat | | LGFTPDTLIPDSAIDD | |
| | | gcacttgaggatactattttgaagttggtcggtgacaca | | FFDYIHKTNAGTLA | |
| | | cctacgtggttttacgctaagtcccttggcttcactcca | | WFVTLSLEGGAINS | |
| | | gataccttgatcccagattcggctattgatgatttcttcg | | VSEDATAYGHRDVL | |
| | | actatattcataagactaacgctggtactctggcctggt | | FWFQVFVVNPLGPIS | |
| | | ttgtgaccttatctttggaaggtggcgctataaactccgt | | QTTYDFTNGLYDVL | |
| | | ttcagaagatgctaccgcttatggtcacagagatgtctt | | AQAVPESAGHAYLG | |
| | | gttttggttccaagttttcgttgtcaatcctcttggtccaat | | CPDPKMPDAQRAY | |
| | | ctctcaaacaacatacgacttcactaatggtttgtacga | | WRSNLPRLEDLKGD | |
| | | cgtattggctcaggccgtgcctgaaagcgctggtcat | | LDPKDTFHNPQGVQ | |
| | | gcttaccttggttgtccagatccaaaaatgccagacgc | | VGP | |
| | | tcagcgtgcttactggagaagtaacttacccagattgg | | | |
| | | aggatctgaagggtgatcttgacccaaaggacaccttt | | | |
| | | cacaaccctcaaggtgttcaagtcggtcca | | | |
| t808253 | Library | atgggcaataccacatctatcgctgccggtagagact | 103 | MGNTTSIAAGRDCL | 173 |
| | | gtctggtcagtgctgttggtcctgcacacgtgacgtttc | | VSAVGPAHVTFQDA | |
| | | aggatgctttgctttaccaaactactgctgttgatcccta | | LLYQTTAVDPYNLN | |
| | | taacttaaacataccagtaaccccagccgctgtcactt | | IPVTPAAVTYPQSAE | |
| | | acccacaatccgctgaggaaattgccgctgttgtgaa | | EIAAVVKCASDYDY | |
| | | gtgcgcttcagactacgattataaagtccaagctaggt | | KVQARSGGHSFGNY | |
| | | ctggaggtcatagcttcggtaactacggtctaggtggt | | GLGGQNGAIVVEMK | |
| | | caaaatggtgcaatcgttgttgaaatgaagcacttctct | | HFSQFSMDESTFVAT | |
| | | caattttccatggacgaatcgaccttcgtcgccactatt | | IGPGTTLGDLDTELY | |
| | | ggcccaggtacaacattgggtgatttagataccgaatt | | NTGGRAMAHGICPT | |
| | | gtataatactggtggccgtgctatggcccatggtatttg | | IRTGGHLTVGGLGPT | |
| | | tccaactatcagaaccggtggtcacttgaccgttggtg | | ARQWGLALDHIEEV | |
| | | gattgggtcctactgcaagacaatggggtttagctcttg | | EVVLANSSIVRASNT | |
| | | atcatatcgaagaagttgaggtcgtcttggctaactctt | | QNQDILFAVKGAAA | |
| | | ccattgttagagctagcaacactcagaaccaagacatt | | SFGIVTEFKVRTQEA | |
| | | ctatttgctgttaaaggagccgctgccagcttcggtata | | PGLAVQYSFTFNLGS | |
| | | gtcaccgaatttaaggttagaacacaggaagctccag | | AAQKAKLVKDWQA | |
| | | gtttggctgtacaatacagtttcaccttcaatttgggctc | | FIAQENLSWKFYSNL | |
| | | agcagctcaaaaggcaaagttggtcaaagactggca | | VIFDGQIILEGIFFGS | |
| | | agccttcatcgctcaagaaaatttatcttggaaattttact | | KEEYDELDLEKRFPT | |
| | | ctaacctagttatttttgacggacaaattatcttggaagg | | SEPGTVLVLTDWLG | |
| | | tatcttcttcggttccaaggaggaatacgatgaactaga | | MIGHGLEDTILKLVG | |
| | | cttagaaaagagattcccaacttctgaaccaggtaccg | | DTPTWFYAKSLGFT | |
| | | tgttggttttaactgattggttgggtatgatcggtcacgg | | PDTLIPDSAIDDFFD | |
| | | tctggaagacactatattgaagttagttggtgatacccc | | YIHKTNAGTLAWFV | |
| | | tacttggttctatgcaaagtccttgggttttacgccagat | | TLSLEGGAINSVSED | |
| | | actttgatacccgattctgccattgacgattttttcgattat | | ATAYGHRDVLFWF | |
| | | attcataagacaaatgctggaaccttggcttggtttgta | | QVFVVNPLGPISQTT | |
| | | acgctatctttggaaggtggtgctataaactctgtctcg | | YDFTNGLYDVLAQA | |
| | | gaagacgcaacagcttacggtcacagagatgtcctgt | | VPESAGHAYLGCPD | |
| | | tttggttccaagtgtttgtagtcaaccctttgggtccaatt | | PKMPDAQRAYWRS | |
| | | tcccagaccacttacgacttcaccaatggtttatacgat | | NLPRLEDLKGDLDP | |
| | | gttcttgctcaagccgttccagaatcggccggccacg | | KDTFHNPQGVQVGP | |
| | | cttatttgggttgtccagaccctaaaatgcccgacgca | | | |
| | | caacgtgcttactggaggtccaacctaccaagattgg | | | |
| | | aggacttaaagggtgacctagacccaaaggatactttt | | | |
| | | cataacccacaaggtgtccaagttggacca | | | |

It should be appreciated that sequences disclosed in this application may or may not contain signal sequences. The sequences disclosed in this application encompass versions with or without signal sequences. It should also be understood that protein sequences disclosed in this application may be depicted with or without a start codon (M). The sequences disclosed in this application encompass versions with or without start codons. Accordingly, in some instances amino acid numbering may correspond to protein sequences containing a start codon, while in other instances, amino acid numbering may correspond to protein sequences that do not contain a start codon. It should also be understood that sequences disclosed in this application may be depicted with or without a stop codon. The sequences disclosed in this application encompass versions with or without stop codons. Aspects of the disclosure encompass host cells comprising any of the sequences described in this application and fragments thereof.

### EQUIVALENTS

Those skilled in the art will recognize, or be able to ascertain using no more than routine experimentation, many equivalents to the specific embodiments of the invention described here. Such equivalents are intended to be encompassed by the following claims.

All references, including patent documents, are incorporated by reference in their entirety.

1. A host cell that comprises a heterologous polynucleotide encoding a terminal synthase (TS), wherein the TS comprises a sequence that is at least 90% identical to SEQ ID NO: 27 or 25 and wherein the host cell is capable of producing at least one cannabinoid.

2. The host cell of claim 1, wherein relative to the sequence of SEQ ID NO: 27, the TS comprises an amino acid substitution at a residue corresponding to position 33, 39, 55, 57, 61, 62, 63, 71, 112, 122, 126, 129, 131 180, 183, 202, 256, 257, 260, 287, 295, 341, 386, 392, 394, 398, 410, 423, 426, 450, and/or 472 of SEQ ID NO: 27.

3. The host cell of claim 2, wherein the TS comprises:
(i) the amino acid D at a residue corresponding to position 33 in SEQ ID NO: 27;
(ii) the amino acid F at a residue corresponding to position 39 in SEQ ID NO: 27;
(iii) the amino acid S at a residue corresponding to position 55 in SEQ ID NO: 27;
(iv) the amino acid Q or E at a residue corresponding to position 57 in SEQ ID NO: 27;
(v) the amino acid A at a residue corresponding to position 61 in SEQ ID NO: 27;
(vi) the amino acid I at a residue corresponding to position 62 in SEQ ID NO: 27;
(vii) the amino acid I at a residue corresponding to position 63 in SEQ ID NO: 27;
(viii) the amino acid I at a residue corresponding to position 71 in SEQ ID NO: 27;
(ix) the amino acid V or T at a residue corresponding to position 112 in SEQ ID NO: 27;
(x) the amino acid S, G, A or E at a residue corresponding to position 122 in SEQ ID NO: 27;
(xi) the amino acid A, R, T, K, or D at a residue corresponding to position 126 in SEQ ID NO: 27;
(xii) the amino acid W at a residue corresponding to position 129 in SEQ ID NO: 27;
(xiii) the amino acid S at a residue corresponding to position 131 in SEQ ID NO: 27;
(xiv) the amino acid T at a residue corresponding to position 180 in SEQ ID NO: 27;
(xv) the amino acid T at a residue corresponding to position 183 in SEQ ID NO: 27;
(xvi) the amino acid S or G at a residue corresponding to position 202 in SEQ ID NO: 27;
(xvii) the amino acid F or M at a residue corresponding to position 256 in SEQ ID NO: 27;
(xviii) the amino acid S at a residue corresponding to position 257 in SEQ ID NO: 27;
(xix) the amino acid M or F at a residue corresponding to position 260 in SEQ ID NO: 27;
(xx) the amino acid R at a residue corresponding to position 287 in SEQ ID NO: 27;
(xxi) the amino acid S at a residue corresponding to position 295 in SEQ ID NO: 27;
(xxii) the amino acid S at a residue corresponding to position 341 in SEQ ID NO: 27;
(xxiii) the amino acid A at a residue corresponding to position 386 in SEQ ID NO: 27;
(xxiv) the amino acid H at a residue corresponding to position 392 in SEQ ID NO: 27;
(xxv) the amino acid T at a residue corresponding to position 394 in SEQ ID NO: 27;
(xxvi) the amino acid F, T, A, or L at a residue corresponding to position 398 in SEQ ID NO: 27;
(xxvii) the amino acid N at a residue corresponding to position 410 in SEQ ID NO: 27;
(xxviii) the amino acid A at a residue corresponding to position 423 in SEQ ID NO: 27;
(xxix) the amino acid Y at a residue corresponding to position 426 in SEQ ID NO: 27;
(xxx) the amino acid K at a residue corresponding to position 450 in SEQ ID NO: 27; and/or
(xxxi) the amino acid R or A at a residue corresponding to position 472 in SEQ ID NO: 27.

4. The host cell of any one of claims 1-3, wherein the TS comprises one or more of the following amino acid substitutions relative to the sequence of SEQ ID NO: 27: T33D; Y39F; T55S; A57Q; A57E; G61A; V62I; V63I; Y71I; E112V; E112T; N122S; N122G; N122A; N122E; I126A; I126R; I126T; I126K; I126D; Y129W; N131S; S180T; R183T; N202S; N202G; Y256F; Y256M; N257S; V260M; V260F; H287R; N295S; A341S; V386A; L392H; M394T; V398F; V398T; V398A; V398L; D410N; S423A; H426Y; R450K; P472R; and/or P472A.

5. The host cell of any one of claims 1-4, wherein the cannabinoid is a CBC-type cannabinoid.

6. The host cell of claim 5, wherein the cannabinoid is cannabichromenic acid (CBCA) and/or cannabichromevarinic acid (CBCVA).

7. The host cell of claim 6, wherein the host cell further produces one or more of tetrahydrocannabinolic acid (THCA), cannabidiolic acid (CBDA) and/or tetrahydrocannabivarinic acid (THCVA).

8. The host cell of any one of claims 2-7, wherein the TS produces a higher ratio of CBCA:CBDA, CBCA:THCA, and/or CBCVA:THCVA than a control TS.

9. The host cell of claim 8, wherein the control TS is a TS comprising the sequence of SEQ ID NO: 20, 23, 25 or 27.

10. The host cell of any one of claims 2-9, wherein the TS comprises one or more of the following amino acid substitutions relative to SEQ ID NO: 27: A57Q and G61A; Y71I; and/or V260F.

11. The host cell of any one of 2-10, wherein the TS has a higher product specificity for a CBC-type cannabinoid than a control TS.

12. The host cell of claim 11, wherein the control TS is a TS comprising the sequence of SEQ ID NO: 20, 23, 25 or 27.

13. The host cell of any one of claims 1-7, wherein the TS comprises Y39F and/or V63I relative to the sequence of SEQ ID NO: 27.

14. The host cell of any one of claims 1 and 5-7, wherein the TS comprises the sequence of any one of SEQ ID NOs: 25, 27, 105, 126, 134, 155, 162, 164, or 165, optionally wherein relative to the sequence of SEQ ID NO: 27, the TS comprises an amino acid substitution at a residue corresponding to position 33, 39, 55, 57, 61, 62, 63, 71, 112, 122, 126, 129, 131 180, 183, 202, 256, 257, 260, 287, 295, 341, 386, 392, 394, 398, 410, 423, 426, 450, and/or 472 of SEQ ID NO: 27.

15. The host cell of any one of claims 1-14, wherein the sequence of the TS comprises one or more of the following motifs:
(i)
(SEQ ID NO: 174)
KVQARSGGH;
(ii)
(SEQ ID NO: 176)
RASNTQNQD[VI][FL]FA[VI]K;
(iii)
(SEQ ID NO: 181)
CPTI[KR]TGGH;
(iv)
(SEQ ID NO: 184)
WFVTLSLEGGAINDV[AP]EDATAY[AG]H;
(v)
(SEQ ID NO: 186)
P[IV]S[DQE]TTY[EDG]F[TA]DGLYDVLA[RQK]AVPES[VA]
GHAYLGCPDP[RK]M;
(vi)
(SEQ ID NO: 189)
MKHF[TNS]QFSM;
(vii)
(SEQ ID NO: 193)
P[EQ][TS]A[EAD][QE]IA[GA][VI]VKC;
(viii)
(SEQ ID NO: 200)
RDCL[IV]SA[LV]GGN[SA]A[LH][AV][AV]F[PQ][ND][QE]
LL[WY];
(ix)
(SEQ ID NO: 207)
RT[EQ][PQ]APGLAVQYSY;
and/or
(x)
(SEQ ID NO: 211)
WQ[SA]FI[SA][AQ][KE]NLT[RW][QK]FY[NST]NM.

16. A host cell for producing a cannabinoid, wherein the host cell comprises a heterologous polynucleotide encoding a terminal synthase (TS), wherein the sequence of the TS comprises one or more of the following motifs:
(i) KVQARSGGH (SEQ ID NO: 174);
(ii) RASNTQNQD[VI][FL]FA[VI]K (SEQ ID NO: 176);
(iii) CPTI[KR]TGGH (SEQ ID NO: 181);
(iv) WFVTLSLEGGAINDV[AP]EDATAY[AG]H (SEQ ID NO: 184);
(v) P[IV]S[DQE]TTY[EDG]F[TA]DGLYDVLA[RQK]AVPES[VA]GHAYLGC PDP[RK]M (SEQ ID NO: 186);
(vi) MKHF[TNS]QFSM (SEQ ID NO: 189);
(vii) P[EQ][TS]A[EAD][QE]IA[GA][VI]VKC (SEQ ID NO: 193);
(viii) RDCL[IV]SA[LV]GGN[SA]A[LH][AV][AV]F[PQ][ND][QE]LL[WY] (SEQ ID NO: 200);
(ix) RT[EQ][PQ]APGLAVQYSY (SEQ ID NO: 207); and/or
(x) WQ[SA]FI[SA][AQ][KE]NLT[RW][QK]FY[NST]NM (SEQ ID NO: 211); and
wherein the host cell is capable of producing at least one cannabinoid.

17. The host cell of claim 16, wherein:
(i) the motif KVQARSGGH (SEQ ID NO: 174) is located at residues in the TS corresponding to residues 72-80 in SEQ ID NO: 27;
(ii) the motif RASNTQNQD[VI][FL]FA[VI]K (SEQ ID NO: 176) is located at residues in the TS corresponding to residues 183-197 in SEQ ID NO: 27;
(iii) the motif CPTI[KR]TGGH (SEQ ID NO: 181) is located at residues in the TS corresponding to residues 141-149 in SEQ ID NO: 27;
(iv) the motif WFVTLSLEGGAINDV[AP]EDATAY[AG]H (SEQ ID NO: 184) is located at residues in the TS corresponding to residues 360-383 in SEQ ID NO: 27;
(v) the motif P[IV]S[DQE]TTY[EDG]F[TA]DGLYDVLA[RQK]AVPES[VA]GHAYLGCPDP[R K]M (SEQ ID NO: 186) is located at residues in the TS corresponding to residues 400-436 in SEQ ID NO: 27;
(vi) the motif MKHF[TNS]QFSM (SEQ ID NO: 189) is located at residues in the TS corresponding to residues 98-106 in SEQ ID NO: 27;
(vii) the motif P[EQ][TS]A[EAD][QE]IA[GA][VI]VKC (SEQ ID NO: 193) is located at residues in the TS corresponding to residues 53-65 in SEQ ID NO: 27;
(viii) the motif RDCL[IV]SA[LV]GGN[SA]A[LH][AV][AV]F[PQ][ND][QE]LL[WY] (SEQ ID NO: 200) is located at residues in the TS corresponding to residues 10-32 in SEQ ID NO: 27;
(ix) the motif RT[EQ][PQ]APGLAVQYSY (SEQ ID NO: 207) is located at residues in the TS corresponding to residues 212-225 in SEQ ID NO: 27; and/or
(x) the motif WQ[SA]FI[SA][AQ][KE]NLT[RW][QK]FY[NST]NM (SEQ ID NO: 211) is located at residues in the TS corresponding to residues 242-259 in SEQ ID NO: 27.

18. The host cell of claim 16 or 17, wherein the TS is a fungal TS or a conservatively substituted version thereof.

19. The host cell of claim 18, wherein the TS is an Apergillus TS or a conservatively substituted version thereof.

20. The host cell of any one of claims 16-19, wherein the TS comprises a sequence that is at least 90% identical to any one of SEQ ID NOs: 25, 27, 105, 112, 126, 130, 134, 144, 155, 159, 162-167, or 172.

21. The host cell of claim 20, wherein relative to the sequence of SEQ ID NO: 27, the TS comprises an amino acid substitution at a residue corresponding to position 33, 39, 55, 57, 61, 62, 63, 71, 112, 122, 126, 129, 131 180, 183, 202, 256, 257, 260, 287, 295, 341, 386, 392, 394, 398, 410, 423, 426, 450, and/or 472 of SEQ ID NO: 27.

22. The host cell of claim 21, wherein the TS comprises:
(i) the amino acid D at a residue corresponding to position 33 in SEQ ID NO: 27;
(ii) the amino acid F at a residue corresponding to position 39 in SEQ ID NO: 27;
(iii) the amino acid S at a residue corresponding to position 55 in SEQ ID NO: 27;
(iv) the amino acid Q or E at a residue corresponding to position 57 in SEQ ID NO: 27;
(v) the amino acid A at a residue corresponding to position 61 in SEQ ID NO: 27;
(vi) the amino acid I at a residue corresponding to position 62 in SEQ ID NO: 27;
(vii) the amino acid I at a residue corresponding to position 63 in SEQ ID NO: 27;
(viii) the amino acid I at a residue corresponding to position 71 in SEQ ID NO: 27;
(ix) the amino acid V or T at a residue corresponding to position 112 in SEQ ID NO: 27;
(x) the amino acid S, G, A or E at a residue corresponding to position 122 in SEQ ID NO: 27;
(xi) the amino acid A, R, T, K, or D at a residue corresponding to position 126 in SEQ ID NO: 27;
(xii) the amino acid W at a residue corresponding to position 129 in SEQ ID NO: 27;
(xiii) the amino acid S at a residue corresponding to position 131 in SEQ ID NO: 27;
(xiv) the amino acid T at a residue corresponding to position 180 in SEQ ID NO: 27;
(xv) the amino acid T at a residue corresponding to position 183 in SEQ ID NO: 27;
(xvi) the amino acid S or G at a residue corresponding to position 202 in SEQ ID NO: 27;
(xvii) the amino acid F or M at a residue corresponding to position 256 in SEQ ID NO: 27;
(xviii) the amino acid S at a residue corresponding to position 257 in SEQ ID NO: 27;
(xix) the amino acid M or F at a residue corresponding to position 260 in SEQ ID NO: 27;
(xx) the amino acid R at a residue corresponding to position 287 in SEQ ID NO: 27;
(xxi) the amino acid S at a residue corresponding to position 295 in SEQ ID NO: 27;
(xxii) the amino acid S at a residue corresponding to position 341 in SEQ ID NO: 27;
(xxiii) the amino acid A at a residue corresponding to position 386 in SEQ ID NO: 27;
(xxiv) the amino acid H at a residue corresponding to position 392 in SEQ ID NO: 27;
(xxv) the amino acid T at a residue corresponding to position 394 in SEQ ID NO: 27;
(xxvi) the amino acid F, T, A, or L at a residue corresponding to position 398 in SEQ ID NO: 27;
(xxvii) the amino acid N at a residue corresponding to position 410 in SEQ ID NO: 27;
(xxviii) the amino acid A at a residue corresponding to position 423 in SEQ ID NO: 27;
(xxix) the amino acid Y at a residue corresponding to position 426 in SEQ ID NO: 27;
(xxx) the amino acid K at a residue corresponding to position 450 in SEQ ID NO: 27; and/or
(xxxi) the amino acid R or A at a residue corresponding to position 472 in SEQ ID NO: 27.

23. The host cell of any one of claims 20-22, wherein the TS comprises one or more of the following amino acid substitutions relative to the sequence of SEQ ID NO: 27: T33D; Y39F; T55S; A57Q; A57E; G61A; V62I; V63I; Y71I; E112V; E112T; N122S; N122G; N122A; N122E; I126A; I126R; I126T; I126K; I126D; Y129W; N131S; S180T; R183T; N202S; N202G; Y256F; Y256M; N257S; V260M; V260F; H287R; N295S; A341S; V386A; L392H; M394T; V398F; V398T; V398A; V398L; D410N; S423A; H426Y; R450K; P472R; and/or P472A.

24. The host cell of claim 20 wherein the TS comprises the sequence of any one of SEQ ID NOs: 25, 27, 105, 112, 126, 130, 134, 143, 144, 155, 159, 162-167, or 172 or a conservatively substituted version thereof.

25. A host cell that comprises a heterologous polynucleotide encoding a terminal synthase (TS), wherein the TS comprises a sequence that is at least 90% identical to any one of SEQ ID NOs: 25, 27, 105, 112, 126, 130, 134, 144, 155, 159, 162-167, or 172, wherein the host cell is capable of producing at least one cannabinoid.

26. The host cell of claim 25, wherein the sequence that is at least 90% identical to any one of SEQ ID NOs: 25, 27, 105, 112, 126, 130, 134, 144, 155, 159, 162-167, or 172 is linked to one or more signal peptides.

27. The host cell of claim 26, wherein the sequence that is at least 90% identical to any one of SEQ ID NOs: 25, 27, 105, 112, 126, 130, 134, 144, 155, 159, 162-167, or 172 is linked to a signal peptide that comprises SEQ ID NO: 16 or a sequence that has no more than two amino acid substitutions, insertions, additions, or deletions relative to the sequence of SEQ ID NO: 16.

28. The host cell of claim 26 or 27, wherein the signal peptide is linked to the N-terminus of the sequence that is at least 90% identical to any one of SEQ ID NOs: 25, 27, 105, 112, 126, 130, 134, 144, 155, 159, 162-167, or 172.

29. The host cell of claim 28, wherein an N-terminal methionine is removed from SEQ ID NOs: 27, 105, 112, 126, 130, 134, 144, 155, 159, 162-167, or 172 and wherein a methionine residue is added to the N-terminus of the signal peptide.

30. The host cell of any one of claims 25-29, wherein the sequence that is at least 90% identical to any one of SEQ ID NOs: 25, 27, 105, 112, 126, 130, 134, 144, 155, 159, 162-167, or 172 is linked to a signal peptide that comprises SEQ ID NO: 17 or a sequence that has no more than one amino acid substitution, insertion, addition, or deletion relative to the sequence of SEQ ID NO: 17.

31. The host cell of claim 30, wherein the signal peptide that comprises SEQ ID NO: 17 or a sequence that has no more than one amino acid substitution, insertion, addition, or deletion relative to the sequence of SEQ ID NO: 17 is linked to the C-terminus of the sequence that is at least 90% identical to any one of SEQ ID NOs: 25, 27, 105, 112, 126, 130, 134, 144, 155, 159, 162-167, or 172.

32. The host cell of any one of claims 25-31, wherein relative to the sequence of SEQ ID NO: 27, the TS comprises an amino acid substitution at a residue corresponding to position 33, 39, 55, 57, 61, 62, 63, 71, 112, 122, 126, 129, 131 180, 183, 202, 256, 257, 260, 287, 295, 341, 386, 392, 394, 398, 410, 423, 426, 450, and/or 472 of SEQ ID NO: 27.

33. The host cell of claim 32, wherein the TS comprises:
(i) the amino acid D at a residue corresponding to position 33 in SEQ ID NO: 27;
(ii) the amino acid F at a residue corresponding to position 39 in SEQ ID NO: 27;
(iii) the amino acid S at a residue corresponding to position 55 in SEQ ID NO: 27;
(iv) the amino acid Q or E at a residue corresponding to position 57 in SEQ ID NO: 27;
(v) the amino acid A at a residue corresponding to position 61 in SEQ ID NO: 27;
(vi) the amino acid I at a residue corresponding to position 62 in SEQ ID NO: 27;
(vii) the amino acid I at a residue corresponding to position 63 in SEQ ID NO: 27;
(viii) the amino acid I at a residue corresponding to position 71 in SEQ ID NO: 27;
(ix) the amino acid V or T at a residue corresponding to position 112 in SEQ ID NO: 27;
(x) the amino acid S, G, A or E at a residue corresponding to position 122 in SEQ ID NO: 27;
(xi) the amino acid A, R, T, K, or D at a residue corresponding to position 126 in SEQ ID NO: 27;
(xii) the amino acid W at a residue corresponding to position 129 in SEQ ID NO: 27;
(xiii) the amino acid S at a residue corresponding to position 131 in SEQ ID NO: 27;
(xiv) the amino acid T at a residue corresponding to position 180 in SEQ ID NO: 27;
(xv) the amino acid T at a residue corresponding to position 183 in SEQ ID NO: 27;
(xvi) the amino acid S or G at a residue corresponding to position 202 in SEQ ID NO: 27;
(xvii) the amino acid F or M at a residue corresponding to position 256 in SEQ ID NO: 27;
(xviii) the amino acid S at a residue corresponding to position 257 in SEQ ID NO: 27;
(xix) the amino acid M or F at a residue corresponding to position 260 in SEQ ID NO: 27;
(xx) the amino acid R at a residue corresponding to position 287 in SEQ ID NO: 27;
(xxi) the amino acid S at a residue corresponding to position 295 in SEQ ID NO: 27;
(xxii) the amino acid S at a residue corresponding to position 341 in SEQ ID NO: 27;
(xxiii) the amino acid A at a residue corresponding to position 386 in SEQ ID NO: 27;
(xxiv) the amino acid H at a residue corresponding to position 392 in SEQ ID NO: 27;
(xxv) the amino acid T at a residue corresponding to position 394 in SEQ ID NO: 27;
(xxvi) the amino acid F, T, A, or L at a residue corresponding to position 398 in SEQ ID NO: 27;
(xxvii) the amino acid N at a residue corresponding to position 410 in SEQ ID NO: 27;
(xxviii) the amino acid A at a residue corresponding to position 423 in SEQ ID NO: 27;
(xxix) the amino acid Y at a residue corresponding to position 426 in SEQ ID NO: 27;
(xxx) the amino acid K at a residue corresponding to position 450 in SEQ ID NO: 27; and/or
(xxxi) the amino acid R or A at a residue corresponding to position 472 in SEQ ID NO: 27.

34. The host cell of any one of claims 25-33, wherein the TS comprises one or more of the following amino acid substitutions relative to the sequence of SEQ ID NO: 27: T33D; Y39F; T55S; A57Q; A57E; G61A; V62I; V63I; Y71I; E112V; E112T; N122S; N122G; N122A; N122E; I126A; I126R; I126T; I126K; I126D; Y129W; N131S; S180T; R183T; N202S; N202G; Y256F; Y256M; N257S; V260M; V260F; H287R; N295S; A341S; V386A; L392H; M394T; V398F; V398T; V398A; V398L; D410N; S423A; H426Y; R450K; P472R; and/or P472A.

35. The host cell of any one of claims 25-34, wherein the heterologous polynucleotide comprises a sequence that is at least 90% identical to any one of SEQ ID NOs: 26, 28, 35, 42, 56, 60, 64, 74, 85, 89, 92, 93, 94, 95, 96, 97, and 102.

36. The host cell of any one of claims 25-31 or 35, wherein the TS sequence comprises any one of SEQ ID NOs: 25, 27, 105, 112, 126, 130, 134, 144, 155, 159, 162-167 and 172.

37. A host cell that comprises a heterologous polynucleotide encoding a terminal synthase (TS), wherein the TS comprises a sequence that is at least 90% identical to any one of SEQ ID NOs: 25, 27, 105, 112, 126, 130, 134, 144, 155, 159, 162-167, or 172, or wherein the host cell comprises a conservatively substituted version of any one of SEQ ID NOs: 25, 27, 105, 112, 126, 130, 134, 144, 155, 159, 162-167, or 172.

38. A host cell that comprises a heterologous polynucleotide encoding a terminal synthase (TS), wherein the host cell is capable of producing at least one cannabinoid, and wherein the TS is a fungal TS or a conservatively substituted version thereof.

39. The host cell of claim 38, wherein the fungal TS is an Aspergillus TS or a conservatively substituted version thereof.

40. The host cell of any one of claims 16-39, wherein the cannabinoid is a is a CBC-type cannabinoid.

41. The host cell of claim 40, wherein the cannabinoid is cannabichromenic acid (CBCA) and/or cannabichromevarinic acid (CBCVA).

42. The host cell of claim 41, wherein the host cell further produces one or more of tetrahydrocannabinolic acid (THCA), cannabidiolic acid (CBDA) and/or tetrahydrocannabivarinic acid (THCVA).

43. The host cell of any one of claims 1-42, wherein the host cell is a plant cell, an algal cell, a yeast cell, a bacterial cell, or an animal cell.

44. The host cell of claim 43, wherein the host cell is a yeast cell.

45. The host cell of claim 44, wherein the yeast cell is a Saccharomyces cell, a Yarrowia cell, a Komagataella cell, or a Pichia cell.

46. The host cell of claim 45, wherein the Saccharomyces cell is a Saccharomyces cerevisiae cell.

47. The host cell of claim 43, wherein the host cell is a bacterial cell.

48. The host cell of claim 47, wherein the bacterial cell is an E. coli cell.

49. The host cell of any one of claims 1-48, wherein the host cell further comprises one or more heterologous polynucleotides encoding one or more of: an acyl activating enzyme (AAE), a polyketide synthase (PKS), a polyketide cyclase (PKC), a prenyltransferase (PT), and/or an additional terminal synthase (TS).

50. The host cell of claim 49, wherein the PKS is an olivetol synthase (OLS) or a divarinol synthase.

51. A method comprising culturing the host cell of any one of claims 1-50.

52. A method for producing a cannabinoid comprising contacting a CBG-type cannabinoid with a terminal synthase (TS), wherein the TS comprises a sequence that is at least 90% identical to any one of SEQ ID NOs: 25, 27, 105, 112, 126, 130, 134, 144, 155, 159, 162-167, or 172.

53. The method of claim 52, wherein contacting the CBG-type cannabinoid with the TS occurs in vitro.

54. The method of claim 52 or 53, wherein contacting the CBG-type cannabinoid with the TS occurs in vivo.

55. The method of claim 54, wherein contacting the CBG-type cannabinoid with the TS occurs in a host cell.

56. A method for producing a cannabinoid comprising contacting a CBG-type cannabinoid in vivo with an oxidative cyclization catalyst adapted to preferentially convert the CBG-type cannabinoid to a CBC-type cannabinoid as compared to a CBD-type cannabinoid, a THC-type cannabinoid or both.

57. The method of any of claims 52-56, wherein the cannabinoid is a cyclized product of a CBG-type cannabinoid.

58. The method of claim 57, wherein the cannabinoid is a cannabinoid with a cyclized prenyl moiety.

59. The method of claim 58, wherein the cannabinoid is a CBC-type cannabinoid, a CBD-type cannabinoid, or a THC-type cannabinoid.

60. The method of claim 59, wherein the cannabinoid is a CBC-type cannabinoid.

61. The method of any one of claims 52-60, wherein the CBG-type cannabinoid is cannabigerolic acid.

62. The method of claim 60, wherein the CBC-type cannabinoid is CBCA.

63. The method of any one of claims 52-62, wherein the TS comprises the sequence of any one of SEQ ID NOs: 25, 27, 105, 112, 126, 130, 134, 144, 155, 159, 162-167, or 172 or a conservatively substituted version thereof.

64. A host cell comprising a CBG-type cannabinoid and a means for catalyzing the oxidative cyclization of the CBG-type cannabinoid to preferentially convert the CBG-type cannabinoid to a CBC-type cannabinoid as compared to a CBG-type cannabinoid, a THC-type cannabinoid, or both.

65. A host cell comprising a CBG-type cannabinoid and an oxidative cyclization catalyst adapted to preferentially convert the CBG-type cannabinoid to a CBC-type cannabinoid as compared to a CBG-type cannabinoid, a THC-type cannabinoid, or both.

66. The host cell of claim 65, wherein the means for catalyzing the oxidative cyclization of the CBG-type cannabinoid to produce a CBC-type cannabinoid is a heterologous polynucleotide encoding a terminal synthase (TS), wherein the TS comprises a sequence that is at least 90% identical to any of SEQ ID NOs: 25, 27, 105, 112, 126, 130, 134, 144, 155, 159, 162-167, or 172 or a conservatively substituted version thereof.

67. The host cell of claim 66, wherein the TS is also capable of producing THCA, THCVA or CBDA.

68. A non-naturally occurring nucleic acid encoding a terminal synthase (TS), wherein the non-naturally occurring nucleic acid comprises a sequence that has at least 90% identity to any one of SEQ ID NOs: 26, 28, 35, 42, 56, 60, 64, 74, 85, 89, 92, 93, 94, 95, 96, 97, and 102.

69. A vector comprising the non-naturally occurring nucleic acid of claim 68.

70. An expression cassette comprising the non-naturally occurring nucleic acid of claim 68.

71. A host cell transformed with the non-naturally occurring nucleic acid of claim 68, the vector of claim 69, or the expression cassette of claim 70.

72. A bioreactor for producing a cannabinoid, wherein the bioreactor contains a CBG-type cannabinoid and a terminal synthase (TS), wherein the TS comprises a sequence that is at least 90% identical to any one of SEQ ID NOs: 25, 27, 105, 112, 126, 130, 134, 144, 155, 159, 162-167, or 172 or wherein the TS comprises a conservatively substituted version of any one of SEQ ID NOs: 25, 27, 105, 112, 126, 130, 134, 144, 155, 159, 162-167, or 172.

73. A non-naturally occurring terminal synthase (TS), wherein the TS comprises a sequence that is at least 90% identical to any one of SEQ ID NOs: 25, 27, 105, 112, 126, 130, 134, 144, 155, 159, 162-167, or 172.

74. An oxidative cyclization catalyst adapted to preferentially convert a CBG-type cannabinoid to a CBC-type compound in vivo as compared to a THC-type compound or a CBD-type compound.
